(19) United States
(12) Patent Application Publication
(10) Pub. No.: US 2004/0106113 A1
(43) Pub. Date: Jun. 3, 2004

(54) Prediction of estrogen receptor status of breast tumors using binary prediction tree modeling

(76) Inventors: Mike West, Durham, NC (US); Joseph R. Nevins, Chapel Hill, NC (US)

Correspondence Address: Gregory J. Glover, Ropes & Gray, Suite 800 East, 1301 K Street, NW, Washington, DC, 20005, US

(21) Appl. No.: 10/291,886

(22) Filed: Nov. 12, 2002

Related U.S. Application Data
(60) Provisional application No. 60/420,729, filed on Oct. 24, 2002.
(60) Provisional application No. 60/421,062, filed on Oct. 25, 2002.
(60) Provisional application No. 60/424,718, filed on Nov. 8, 2002.
(60) Provisional application No. 60/424,715, filed on Nov. 8, 2002.

(51) Int. Cl. C12Q 1/68; G06F 19/00; G01N 33/48; G01N 33/50

(52) U.S. Cl. 435/6; 702/20

(57) ABSTRACT

The statistical analysis described and claimed is a predictive statistical tree model that overcomes several problems observed in prior statistical models and regression analyses, while ensuring greater accuracy and predictive capabilities. Although the claimed use of the predictive statistical tree model described herein is directed to the prediction of estrogen receptor status in individuals, the claimed model can be used for a variety of applications including the prediction of disease states, susceptibility of disease states or any other biological state of interest, as well as other applicable non-biological states of interest. This model first screens genes to reduce noise, applies k-means correlation-based clustering targeting a large number of clusters, and then uses singular value decompositions (SVD) to extract the single dominant factor (principal component) from each cluster. This generates a statistically significant number of cluster-derived singular factors, that we refer to as metagenes, that characterize multiple patterns of expression of the genes across samples. The strategy aims to extract multiple such patterns while reducing dimension and smoothing out gene-specific noise through the aggregation within clusters. Formal predictive analysis then uses these metagenes in a Bayesian classification tree analysis. This generates multiple recursive partitions of the sample into subgroups (the “leaves” of the classification tree), and associates Bayesian predictive probabilities of outcomes with each subgroup. Overall predictions for an individual sample are then generated by averaging predictions, with appropriate weights, across many such tree models. The model includes the use of iterative out-of-sample, cross-validation predictions leaving each sample out of the data set one at a time, refitting the model from the remaining samples and using it to predict the hold-out case. This rigorously tests the predictive value of a model and mirrors the real-world prognostic context where prediction of new cases as they arise is the major goal.

### FIELD OF THE INVENTION

The field of this invention is the application of classification tree models incorporating Bayesian analysis to the statistical prediction of binary outcomes where the binary outcome is estrogen receptor status.

### BACKGROUND OF THE INVENTION

Bayesian analysis is an approach to statistical analysis that is based on the Bayes's law, which states that the posterior probability of a parameter p is proportional to the prior probability of parameter p multiplied by the likelihood of p derived from the data collected. This increasingly popular methodology represents an alternative to the traditional (or frequentist probability) approach: whereas the latter attempts to establish confidence intervals around parameters, and/or falsify a-priori null-hypotheses, the Bayesian approach attempts to keep track of how a-priori expectations about some phenomenon of interest can be refined, and how observed data can be integrated with such a-priori beliefs, to arrive at updated posterior expectations about the phenomenon.

Bayesian analysis have been applied to numerous statistical models to predict outcomes of events based on available data. These include standard regression models, e.g. binary regression models, as well as to more complex models that are applicable to multi-variate and essentially non-linear data. Another such model is commonly known as the tree model which is essentially based on a decision tree. Decision trees can be used in clarification, prediction and regression. A decision tree model is built starting with a root mode, and training data partitioned to what are essentially the “children” modes using a splitting rule. For instance, for clarification, training data contains sample vectors that have one or more measurement variables and one variable that determines that class of the sample. Various splitting rules have been used; however, the success of the predictive ability varies considerably as data sets become larger. Furthermore, past attempts at determining the best splitting for each mode is often based on a “purity” function calculated from the data, where the data is considered pure when it contains data samples only from one clan. Most frequently, used purity functions are entropy, gini-index, and towing rule. The success of each of these tree models varies considerably and their applicability to complex biological and molecular data is often prone to difficulties. Thus, there is a med statistical model that can consistently deliver accurate results with high predictive capabilities. The present invention describes a statistical predictive tree model to which Bayesian analysis is applied incorporating several key innovations described herewith.

### SUMMARY OF THE INVENTION

This invention discusses the generation and exploration of classification tree models, with particular interest in problems involving many predictors. Problems involving multiple predictors arise in situations where the prediction of an outcome is dependent on the interaction of numerous factors (predictors), such as the prediction of clinical or physiological states using various forms of molecular data. One motivating application is molecular phenotyping using gene expression and other forms of molecular data as predictors of a clinical or physiological state.

The invention addresses the specific context of a binary response Z and many predictors xi; in which the data arises via case-control design, i.e., the numbers of 0/1 values in the response data are fixed by design. This allows for the successful relation of large-scale gene expression data (the predictors) to binary outcomes, such as a risk group or disease state. The invention elaborates on a Bayesian analysis of this particular binary context, with several key innovations. The analysis of this invention addresses and incorporates case-control design issues in the assessment of association between predictors and outcome with nodes of a tree. With categorical or continuous covariates, this is based on an underlying non-parametric model for the conditional distribution of predictor values given outcomes, consistent with the case-control design. This uses sequences of Bayes' factor based tests of association to rank and select predictors that define significant “splits” of nodes, and that provides an approach to forward generation of trees that is generally conservative in generating trees that are effectively self-pruning. An innovative element of the invention is the implementation of a tree-spawning method to generate multiple trees with the aim of finding classes of trees with high marginal likelihoods, and where the prediction is based on model averaging, i.e., weighting predictions of trees by their implied posterior probabilities. The advantage of the Bayesian approach is that rather than identifying a single “best” tree, a score is attached to all possible trees and those trees which are very unlikely are excluded. Posterior and predictive distributions are evaluated at each node and at the leaves of each tree, and feed into both the evaluation and interpretation tree by tree, and the averaging of predictions across trees for future cases to be predicted.

To demonstrate the utility and advantages of this tree classification model, an embodiments is provided that concerns gene expression profiling using DNA microarray data as predictors of a clinical states in breast cancer. The clinical state is estrogen receptor (“ER”) prediction. The example of ER status prediction demonstrates not only predictive value but also the utility of the tree modeling framework in aiding exploratory analysis that identify multiple, related aspects of gene expression patterns related to a binary outcome, with some interesting interpretation and insights. This embodiment also illustrates the use of metagene factors—multiple, aggregate measures of complex gene expression patterns—in a predictive modeling context. In the case of large numbers of candidate predictors, in particular, model sensitivity to changes in selected subsets of predictors are ameliorated though the generation of multiple trees, and relevant, data-weighted averaging over multiple trees in prediction. The development of formal, simulation-based analyses of such models provides ways of dealing with the issues of high collinearity among multiple subsets of predictors, and challenging computational issues.

### BRIEF DESCRIPTION OF THE FIGURES

FIG. 1: Three ER related metagenes in 49 primary breast tumors. Samples are denoted by blue (ER negative) and red (ER positive), with training data represented by filled circles and validation data by open circles.

FIG. 2: Three ER related metagenes in 49 primary breast tumors. All samples are represented by index number in 1-78. Training data are denoted by blue (ER negative) and red (ER positive), and validation data by cyan (ER negative) and magenta (ER positive).

FIG. 2: Honest predictions of ER status of breast tumors. Predictive probabilities are indicated, for each tumor, by the index number on the vertical probability scale, together with an approximate 90% uncertainty interval about the estimated probability. All probabilities are referenced to a notional initial probability (incidence rate) of 0.5 for comparison. Training data are denoted by blue (ER negative) and red (ER positive), and validation data by cyan (ER negative) and magenta (ER positive).

FIG. 4: Table of 491 ER metagenes in initial (random) order.

FIG. 5: Table of 491 ER metagenes ordered in terms of nonlinear association with ER status.

### DETAILED DESCRIPTION OF THE INVENTION

Development of the Tree Clarification Model: Model Context and Methodology Data {Zi, x₁} (i=1, . . . , n) are available on a binary response variable Z and a p-dimensional covariate vector x: The 0/1 response totals are fixed by design. Each predictor variable xⱼ could be binary, discrete or continuous.

1. Bayes' Factor Measures of Association

At the heart of a classification tree is the assessment of association between each predictor and the response in subsamples, and we first consider this at a general level in the full sample. For any chosen single predictor x; a specified threshold_on the levels of x organizes the data into the 2×2 table.

| | | | |
|---|---|---|---|
| | Z = 0 | Z = 1 | |
| x ≦ τ | n₀₀ | n₀₁ | N₀ |
| x > τ | n₁₀ | n₁₁ | N₁ |
| | M₀ | M₁ | |

With column totals fixed by design, the categorized data is properly viewed as two Bernoulli sequences within the two columns, hence sampling

$$p ( {n}_{0} \text{?} M {\text{?}}_{2 , τ} ) = θ \text{?} {( 1 - {θ}_{z , τ} )}^{{n}_{1 z}}$$

$$\text{?} \text{indicates text missing or illegible when filed}$$

for each column: τ=0.1. Here, of course, 0_{0,τ}−Pr(x≦τ|Z=0) and 0_{1,τ}−Pr(x≦τZ·1). A test of association of the thresholded predictor with the response will now be based on assessing the difference between Bernoulli probabilities.

The natural Bayesian approach is via the Bayes' factor B_{τ} comparing the null hypothesis 0_{0,τ}−0_{1τ}to the full alternative 0_{0.τ}≠0_{1,τ}. We adopt the standard conjugate beta prior model and require that the

densitie

null hypothesis be nested within the alternative. Thus, assuming 0_{0,τ}≠0_{1,τ}, we take 0_{0,τ} and 0_{1,τ} to be independent with common prior Be(α_{τ}, b_{τ}) with mean m_{τ}-α_{τ}/(α_{τ}|b_{τ}). On the null hypothesis 0_{0,τ-0}_{1,τ}, the common value has the same beta prior. The resulting Bayes' factor in favour of the alternative over the null hypothesis is then simply

$${B}_{τ} = \frac{β ( \text{?} + {a}_{τ} {\text{?}}_{10} + {\text{?}}_{τ} ) β ( {\text{?}}_{01} + {a}_{τ} {\text{?}}_{11} + {b}_{τ} )}{β ( {N}_{0} + {a}_{τ} \text{?} {N}_{1} + {b}_{τ} ) β ( {a}_{τ} \text{?} {b}_{τ} )} . \\ \text{?} \text{indicates text missing or illegible when filed}$$

As a Bayes' factor, this is calibrated to a likelihood ratio scale. In contrast to more traditional significance tests and also likelihood ratio approaches, the Bayes' factor will tend to provide more conservative assessments of significance, consistent with the general conservative properties of proper Bayesian tests of null hypotheses (See Sellke, T., Bayarri, M. J. and Berger, J. O., Calibration of p_values for testing precise null hypotheses, The American Statistician, 55, 62-71, (2001) and references therein).

In the context of comparing predictors, the Bayes' factor Bτ may be evaluated for all predictors and, for each predictor, for any specified range of thresholds. As the threshold varies for a given predictor taking a range of (discrete or continuous) values, the Bayes' factor maps out a function of τ and high values identify ranges of interest for thresholding that predictor. For a binary predictor, of course, the only relevant threshold to consider is τ=0.

2. Model Consistency with Respect to Varying Thresholds

A key question arises as to the consistency of this analysis as we vary the thresholds. By construction, each probability θ_{Zτ} is a non-decreasing function of τ, a constraint that must be formally represented in the model. The key point is that the beta prior specification must formally reflect this. To see how this is achieved, note first that θ_{Zτ} is in fact the cumulative distribution function of the predictor values _{X}; conditional on Z=z; (z=0; 1); evaluated at the point _{X}=τ. Hence the sequence of beta priors, Be(α_{τ}, b_{τ}) as τ varies, represents a set of marginal prior distributions for the corresponding, set of values of the cdfs. It is immediate that the natural embedding is in a non-parametric Dirichlet process model for the complete cdf. Thus the threshold-specific beta priors are consistent, and the resulting sets of Bayes' factors comparable as τ varies, under a Dirichlet process prior with the betas as margins. The required constraint is that the prior mean values m_{τ} are themselves values of a cumulative distribution function on the range of _{X}, one that defines the prior mean of each θ_{τ} as a function. Thus, we simply rewrite the beta parameters (α_{τ}, b_{τ}) as α_{τ}=αm_{τ} and b_{τ}=α(1−m_{τ}) for a specified prior mean cdf m_{τ}, and where α is the prior precision (or “total mass”) of the underlying Dirichlet process model. Note that this specialises to a Dirichlet distribution when _{X} is discrete on a finite set of values, including special cases of ordered categories (such as arise if _{X} is truncated to a predefined set of bins), and also the extreme case of binary _{X} when the Dirichlet is a simple beta distribution.

3. Generating a Tree

The above development leads to a formal Bayes' factor measure of association that may be used in the generation of trees in a forward-selection process as implemented in traditional classification tree approaches. Consider a single tree and the data in a node that is a candidate for a binary split. Given the data in this node, construct a binary split based on a chosen (predictor, threshold) pair (_{X}, τ) by (a) finding the (predictor, threshold) combination that maximizes the Bayes' factor for a split, and (b) splitting if the resulting Bayes' factor is sufficiently large. By reference to a posterior probability scale with respect to a notional 50:50 3 prior, Bayes' factors of 2.2,2.9,3.7 and 5.3 correspond, approximately, to probabilities of 0.9, 0.95, 0.99 and 0.995, respectively. This guides the choice of threshold, which may be specified as a single value for each level of the tree. We have utilised Bayes' factor thresholds of around 3 in a range of analyses, as exemplified below. Higher thresholds limit the growth of trees by ensuring a more stringent test for splits.

The Bayes' factor measure will always generate less extreme values than corresponding generalized likelihood ratio tests (for example), and this can be especially marked when the sample sizes M₀ and M₁ are low. Thus the propensity to split nodes is always generally lower than with traditional testing methods, especially with lower samples sizes, and hence the approach tends to be more conservative in extending existing trees. Post-generation pruning is therefore generally much less of an issue, and can in fact generally be ignored.

Index the root node of any tree by zero, and consider the full data set of n observations, representing M_{Z} outcomes with Z=z in 0, 1. Label successive nodes sequentially: splitting the root node, the left branch terminates at node 1, the right branch at node 2; splitting node 1, the consequent left branch terminates at node 3, the right branch at node 4; splitting node 2, the consequent left branch terminates at node 5, and the right branch at node 6, and so forth. Any node in the tree is labelled numerically according to its “parent” node; that is, a node j splits into two children, namely the (left, right) children (2j+1; 2j+2): At level m of the tree (m=0; 1; : : : ;) the candidates nodes are, from left to right, as 2^{m}_{—}1; 2^{m}; : : : ; 2^{m+1}−2.

Having generated a “current” tree, we run through each of the existing terminal nodes one at a time, and assess whether or not to create a further split at that node, stopping based on the above Bayes' factor criterion. Unless samples are very large (thousands) typical trees will rarely extend to more than three or four levels.

4. Inference and Prediction with a Single Tree

Suppose we have generated a tree with m levels; the tree has some number of terminal nodes up to the maximum possible of L=2^{m+1}−2. Inference and prediction involves computations for branch probabilities and the predictive probabilities for new cases that these underlie. We detail this for a specific path down the tree, i.e., a sequence of nodes from the root node to a specified terminal node.

First, consider a node j that is split based on a (predictor, threshold) pair labeled (_{Xj}, τⱼ), (note that we use the node index to label the chosen predictor, for clarity). Extend the notation of Section 2.1 to include the subscript j indexing this node. Then the data at this node involves M₀ⱼ cases with Z=0 and M₁ⱼ cases with Z=1. Based on the chosen (predictor, threshold) pair (_{Xj}, τⱼ) these samples split into cases n₀₀₁ⱼ, n₀₁ⱼ, n₁₁ⱼ as in the table of Section 2.1, but now indexed by the node label j. The implied conditional probabilities θ_{z,τj}=Pr(_{Xj}≦τⱼ|Z=z), for z=0, 1 are the branch probabilities defined by such a split (note that these are also conditional on the tree and data subsample in this node, though the notation does not explicitly reflect this for clarity). These are uncertain parameters and, following the development of Section 2.1, have specified beta priors, now also indexed by parent node j, i.e., Be(a_{τ,j}, b_{τ,j}). Assuming the node is split, the two sample Bernoulli setup implies conditional posterior distributions for these branch probability parameters: they are independent with posterior beta distributions

θ_{0,τj}˜Be(a_{τ,j}+n₀₀ⱼb_{τj}+n₁₀ⱼ) and θ_{1,τj}˜Be(α_{τj}+n₀₁ⱼ,b_{τj}+n₁₁ⱼ).

These distributions allow inference on branch probabilities, and feed into the predictive inference computations as follows.

Consider predicting the response Z* of a new case based on the observed set of predictor values x*. The specified tree defines a unique path from the root to the terminal node for this new case. To predict requires that we compute the posterior predictive probability for Z*=1/0. We do this by following x* down the tree to the implied terminal node, and sequentially building up the relevant likelihood ratio defined by successive (predictor, threshold) pairs.

For example and specificity, suppose that the predictor profile of this new case is such that the implied path traverses nodes 0, 1, 4, 9, terminating at node 9. This path is based on a (predictor, threshold) pair (_{X0}, τ₀) that defines the split of the root node, (_{X1}, τ₁)that defines the split of node 1, and (_{X4}, τ₄) that defines the split of node 4. The new case follows this path as a result of its predictor values, in sequence:

(x′₀≦τ₀), (x′₁>τ₁) and (x′₄≦τ₄). The implied likelihood ratio for Z′-1 relative to Z′-n is then the product of the ratio of branch probabilities to this terminal node, namely

$${λ}^{*} \text{?} \frac{{θ}_{1 , {τ}_{0} , 0}}{{θ}_{0 , {τ}_{0} , 0}} \times \frac{( 1 - {θ}_{1 , {τ}_{1} , 1} )}{( 1 - {θ}_{0 , {τ}_{1} , 1} )} \times \frac{{θ}_{1 , {τ}_{0} , 0}}{{θ}_{0 , {τ}_{0} , 0}} . \\ \text{?} \text{indicates text missing or illegible when filed}$$

Hence, for any specified prior probability Pr(Z′-1), this single tree model implies that, as a function of the branch probabilities, the updated probability τ′ is, on the odds scale, given by

$$\frac{{π}^{*}}{( 1 - {π}^{*} )} = {λ}^{*} \frac{Pr ( {Z}^{*} = 1 )}{Pr ( {Z}^{*} = 0 )} .$$

Hence, for any specified prior probability πPr(Z*=1), this single tree model implies that, as a function the branch probabilities, the updated probability π* is, on the odds scale, given by

$$\frac{{π}^{*}}{( 1 - {π}^{*} )} = {λ}^{*} \frac{Pr ( {Z}^{*} = 1 )}{Pr ( {Z}^{*} = 0 )}$$

The case-control design provides no information about Pr(Z*=1) so it is up to the user to specify this or examine a range of values; one useful summary is obtained by simply taking a 50:50 prior odds as benchmark, whereupon the posterior probability is

π*=λ*/(1+λ*).

Prediction follows by estimating π* based on the sequence of conditionally independent posterior distributions for the branch probabilities that define it. For example, simply “plugging-in” the conditional posterior means of each θ. will lead to a plug-in estimate of λ* and hence π*. The full posterior for π* is defined implicitly as it is a function of the θ. Since the branch probabilities follow beta posteriors, it is trivial to draw Monte Carlo samples of the θ. and then simply compute the corresponding values of λ* and hence π* to generate a posterior sample for summarization. This way, we can evaluate simulation-based posterior means and uncertainty intervals for π* that represent predictions of the binary outcome for the new case.

5. Generating and Weighting Multiple Trees

In considering potential (predictor, threshold) candidates at any node, there may be a number with high Bayes' factors, so that multiple possible trees with difference splits at this node are suggested. With continuous predictor variables, small variations in an “interesting” threshold will generally lead to small changes in the Bayes' factor—moving the threshold so that a single observation moves from one side of the threshold to the other, for example. This relates naturally to the need to consider thresholds as parameters to be inferred; for a given predictor _{X}, multiple candidate splits with various different threshold values τ reflects the inherent uncertainty about τ, and indicates the need to generate multiple trees to adequately represent that uncertainty. Hence, in such a situation, the tree generation can spawn multiple copies of the “current” tree, and then each will split the current node based on a different threshold for this predictor. Similarly, multiple trees may be spawned this way with the modification that they may involve different predictors.

In problems with many predictors, this naturally leads to the generation of many trees, often with small changes from one to the next, and the consequent need for careful development of tree-managing software to represent the multiple trees. In addition, there is then a need to develop inference and prediction in the context of multiple trees generated this way. The use of “forests of trees” has recently been urged by Breiman, L., Statistical Modeling: The two cultures (with discussion), Statistical Science, 16 199-225 (2001), and our perspective endorses this. The rationale here is quite simple: node splits are based on specific choices of what we regard as parameters of the overall predictive tree model, the (predictor, threshold) pairs. Inference based on any single tree chooses specific values for these parameters, whereas statistical learning about relevant trees requires that we explore aspects of the posterior distribution for the parameters (together with the resulting branch probabilities).

Within the current framework, the forward generation process allows easily for the computation of the resulting relative likelihood values for trees, and hence to relevant weighting of trees in prediction. For a given tree, identify the subset of nodes that are split to create branches. The overall marginal likelihood function for the tree is then the product of component marginal likelihoods, one component from each of these split nodes. Continue with the notation of Section 2.1 but now, again, indexed by any chosen node j: Conditional on splitting the node at the defined (predictor, threshold) pair (_{Xj}, τⱼ), the marginal likelihood component is

$${m}_{j} = {\int }_{0}^{1} {\int }_{0}^{1} {\prod }_{z = 0 , 1} p ( {\text{?}}_{0 zj} , \text{?} {\text{?}}_{zj} {\text{?}}_{z , {τ}_{j} , j} ) p ( {θ}_{z , {τ}_{j} , j} ) ⅆ {θ}_{z , {τ}_{j} , j}$$

$$\text{?} \text{indicates text missing or illegible when filed}$$

where p(0_{2,τj}_{^{j}}) is the Be(α_{τ,j},b_{τ,j}) prior for each: τ=0.1. This clearly reduces to

$${m}_{j} = {\prod }_{z = 0 , 1} \frac{β ( {\text{?}}_{0 zj} + {a}_{τ , j} , {n}_{1 zj} + {b}_{τ , j} )}{β ( {a}_{τ , j} , {b}_{τ , j} )} . \\ \text{?} \text{indicates text missing or illegible when filed}$$

The overall marginal likelihood value is the product of these terms over all nodes j that define branches in the tree. This provides the relative likelihood values for all trees within the set of trees generated. As a first reference analysis, we may simply normalise these values to provide relative posterior probabilities over trees based on an assumed uniform prior. This provides a reference weighting that can be used to both assess trees and as posterior probabilities with which to weight and average predictions for future cases.

### DESCRIPTION OF THE SPECIFIC EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

### EXAMPLE 1

### Metagene Expression Profiling to Predict Estrogen Receptor Status of Breast Cancer Tumors

This example illustrates not only predictive utility but also exploratory use of the tree analysis framework in exploring data structure. Here, the tree analysis is used to predict estrogen receptor (“ER”) status of breast tumors using gene expression data. Prior analyses of such data involved binary regression models which utilized Bayesian generalized shrinkage approaches to factor regression. Specifically, prior statistical models involved the use of probit linear regression linking principal components of selected subsets of genes to the binary (ER positive/negative) outcomes. See West, M., Blanchette, C., Dressman, H., Ishida, S., Spang, R., Zuzan, H., Marks, J. R. and Nevins, J. R. Utilization of gene expression profiles to predict the clinical status of human breast cancer. Proc. Natl. Acad. Sci., 98, 11462-11467 (2001). However, the tree model presents some distinct advantages over Bayesian linear regression models in the analysis of large non-linear data sets such as these.

Primary breast tumors from the Duke Breast Cancer SPORE frozen tissue bank were selected for this study on the basis of several criteria. Tumors were either positive for both the estrogen and progesterone receptors or negative for both receptors. Each tumor was diagnosed as invasive ductal carcinoma and was between 1.5 and 5 cm in maximal dimension. In each case, a diagnostic axillary lymph node dissection was performed. Each potential tumor was examined by hematoxylin/eosin staining and only those that were >60% tumor (on a per-cell basis), with few infiltrating lymphocytes or necrotic tissue, were carried on for RNA extraction. The final collection of tumors consisted of 13 estrogen receptor (ER)+lymph node (LN)+tumors, 12 ER LN+tumors, 12 ER+LN tumors, and 12 ER LN tumors

The RNA was derived from the tumors as follows: Approximately 30 mg of frozen breast tumor tissue was added to a chilled BioPulverizer H tube (Bio101) (Q-Biogene, La Jolla, Calif.). Lysis buffer from the Qiagen (Chatsworth, Calif.) RNeasy Mini kit was added, and the tissue was homogenized for 20 sec in a MiniBeadbeater (Biospec Products, Bartlesville, Okla.). Tubes were spun briefly to pellet the garnet mixture and reduce foam. The lysate was transferred to a new 1.5-ml tube by using a syringe and 21-gauge needle, followed by passage through the needle 10 times to shear genomic DNA. Total RNA was extracted by using the Qiagen RNeasy Mini kit.

Two extractions were performed for each tumor, and total RNA was pooled at the end of the RNeasy protocol, followed by a precipitation step to reduce volume. Quality of the RNA was checked by visualization of the 28S:18S ribosomal RNA ratio on a 1% agarose gel. After the RNA preparation, the samples were subject to Affymetrix GENECHIP analysis.

Affymetrix GENECHIP Analysis: The targets for Affymetrix DNA microarray analysis were prepared according to the manufacturer's instructions. All assays used the human HuGeneFL GENECHIP microarray. Arrays were hybridized with the targets at 45° C. for 16 h and then washed and stained by using the GENECHIP Fluidics. DNA chips were scanned with the GENECHIP scanner, and signals obtained by the scanning were processed by GENECHIP Expression Analysis algorithm (version 3.2) (Affymetrix, Santa Clara, Calif.).

The same set of n=49 samples used in the binary regression analysis described in West et al (2001) is analyzed in this study, using predictors based on metagene summaries of the expression levels of many genes. Metagenes are useful aggregate, summary measures of gene expression profiles. The evaluation and summarization of large-scale gene expression data in terms of lower dimensional factors of some form is utilized for two main purposes: first, to reduce dimension from typically several thousand, or tens of thousands of genes to a more practical dimension; second, to identify multiple underlying “patterns” of variation across samples that small subsets of genes share, and that characterize the diversity of patterns evidenced in the full sample. Although, the analysis is conducive to the use of various factor model approaches known to those skilled in the art, a cluster-factor approach is used here to define empirical metagenes. This defines the predictor variables x utilized in the tree model.

Metagenes can be obtained by combining clustering with empirical factor methods. The metagene summaries used in the ER example in this disclosure, are based on the following steps.

Assume a sample of n profiles of p genes;

Screen genes to reduce the number by eliminating genes that show limited variation across samples or that are evidently expressed at low levels that are not detectable at the resolution of the gene expression technology used to measure levels. This removes noise and reduces the dimension of the predictor variable;

Cluster the genes using k_means, correlated-based clustering. Any standard statistical package may be used. This analysis uses the xcluster software created by Gavin Sherlock (http://genomewww.stanford.edu/sherlock/cluster.html). A large number of clusters are targeted so as to capture multiple, correlated patterns of variation across samples, and generally small numbers of genes within clusters;

Extract the dominant singular factor (principal component) from each of the resulting clusters. Again, any standard statistical or numerical software package may be used for this; this analysis uses the efficient, reduced singular value decomposition function (“SVD”) in the Matlab software environment (http://www.mathworks.com/products/matlab).

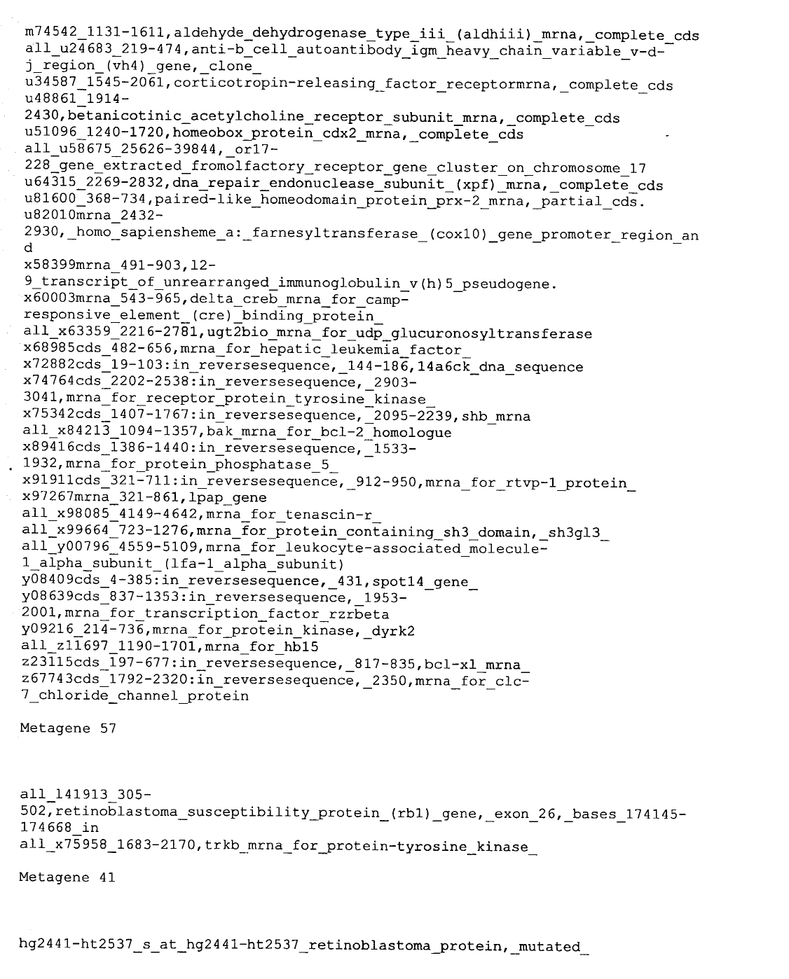

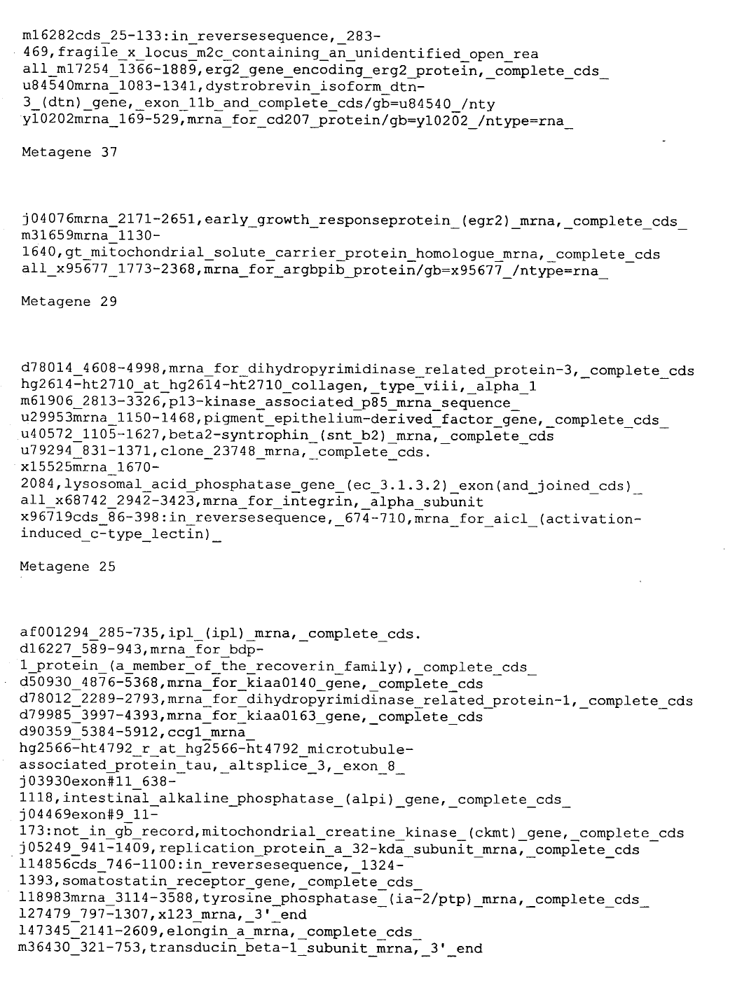

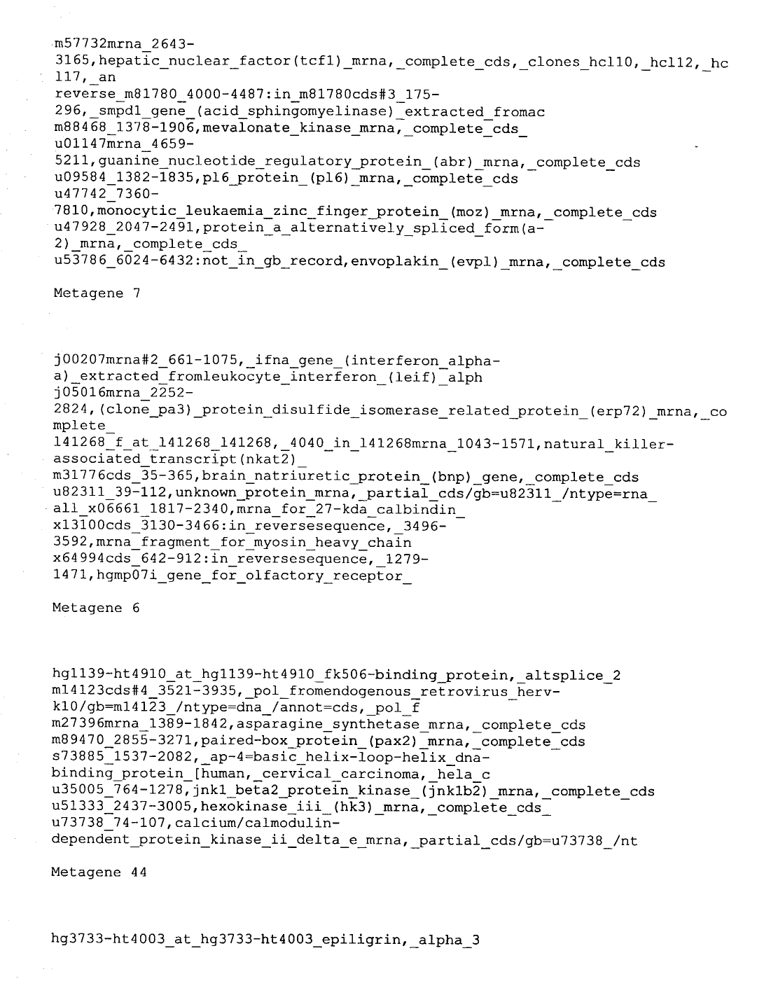

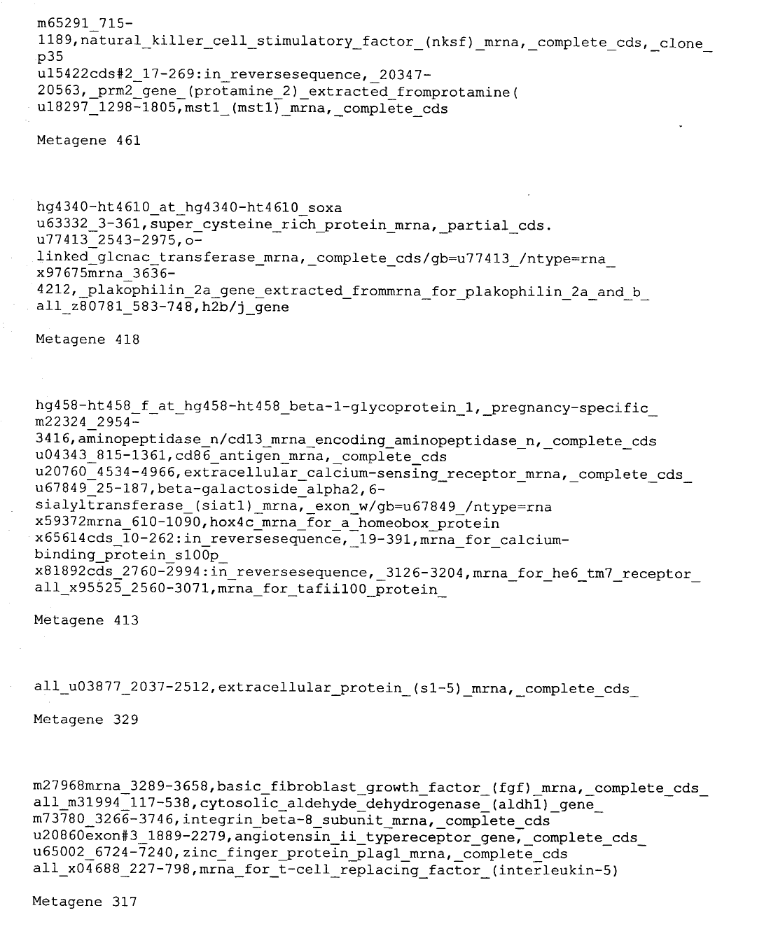

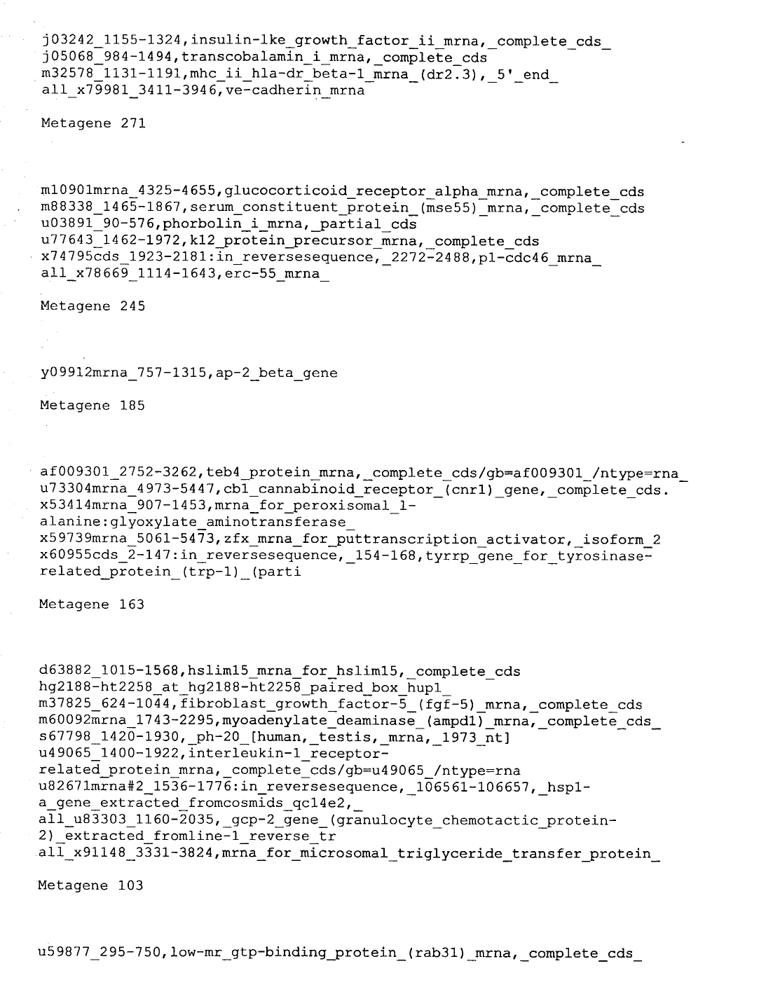

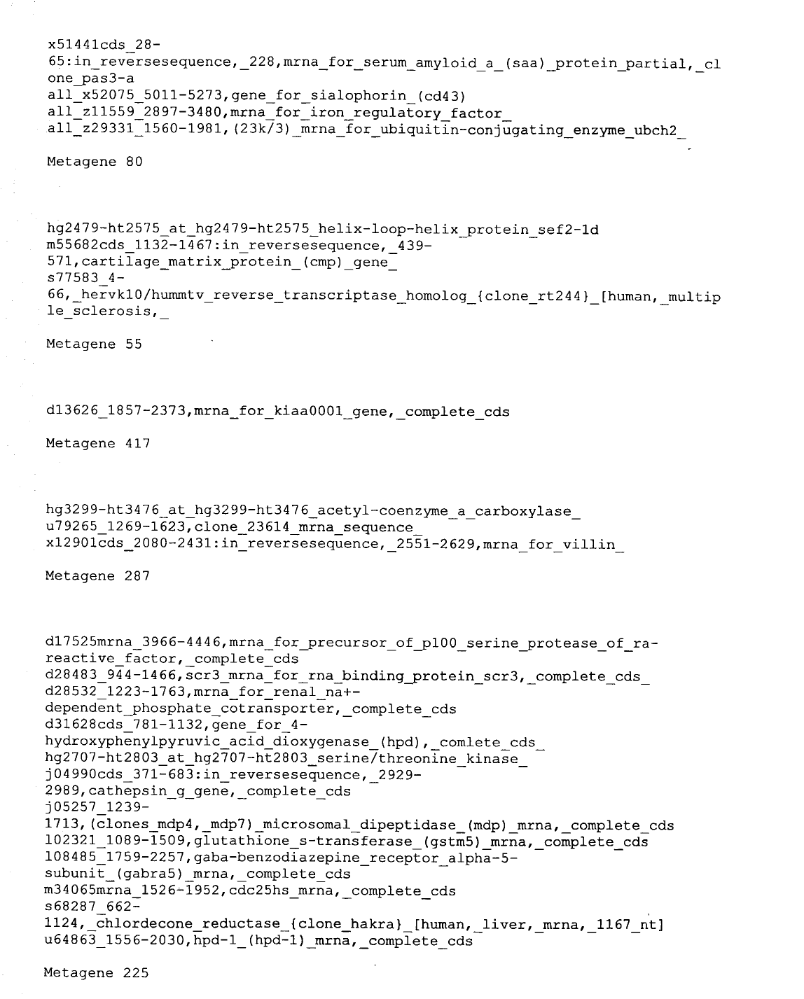

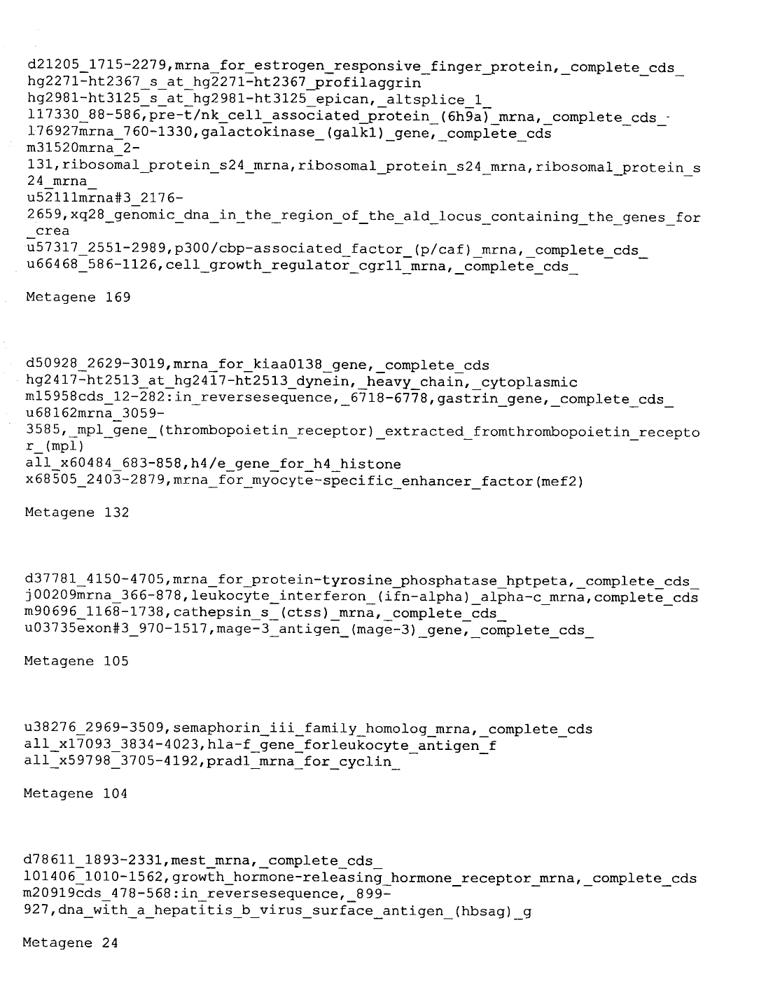

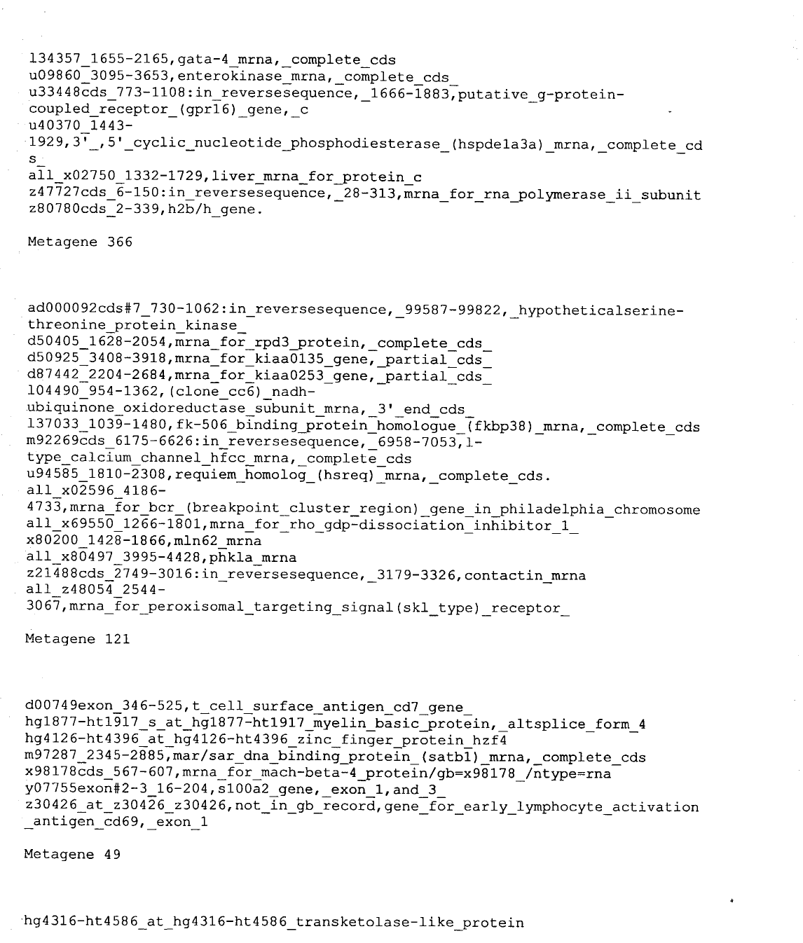

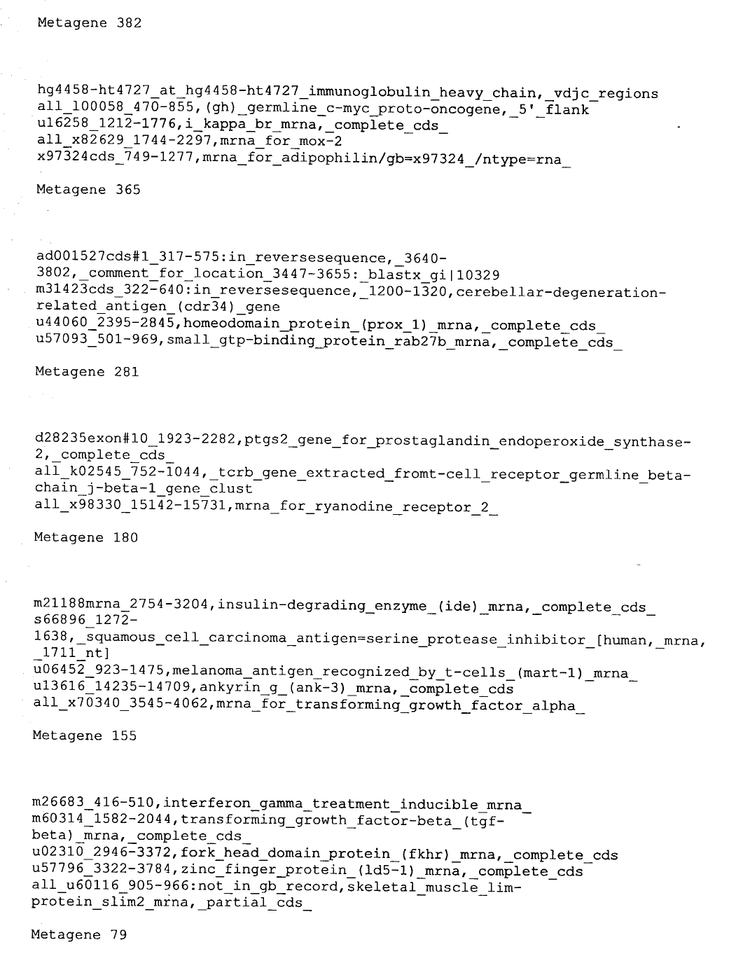

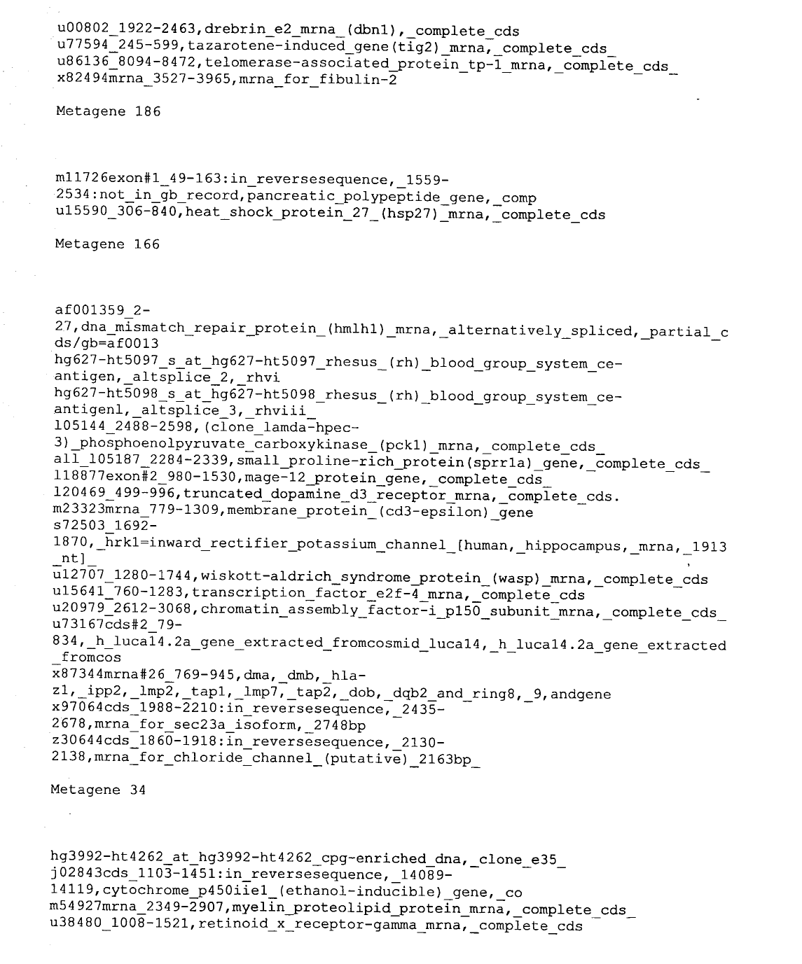

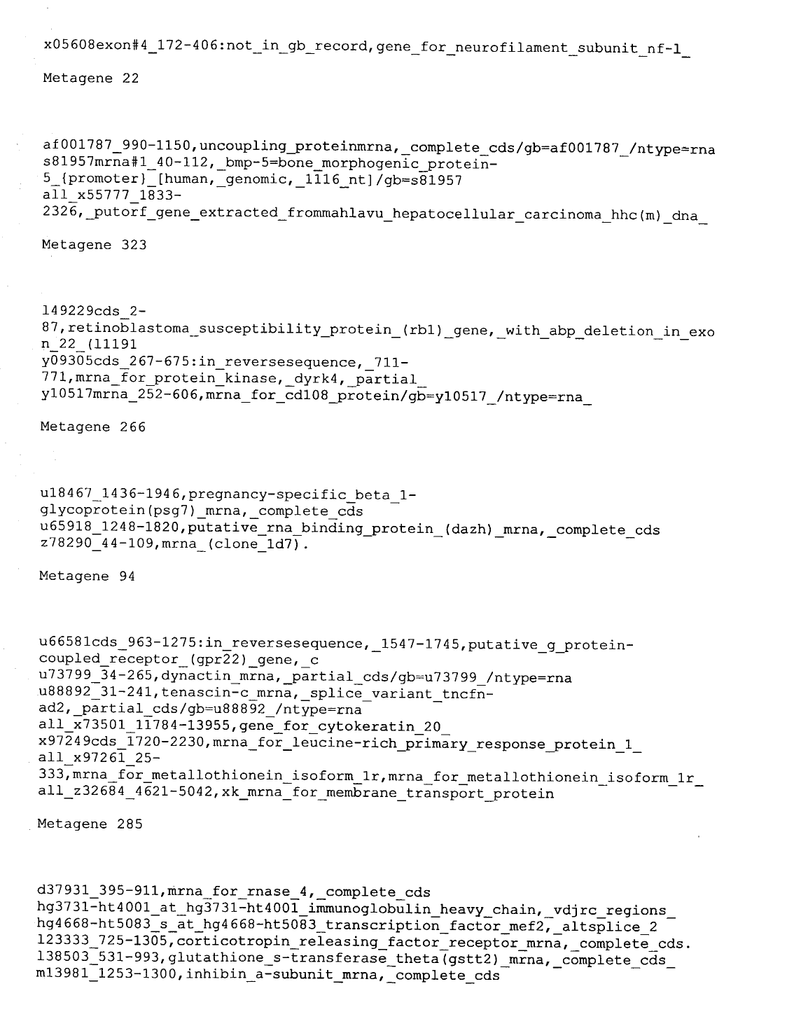

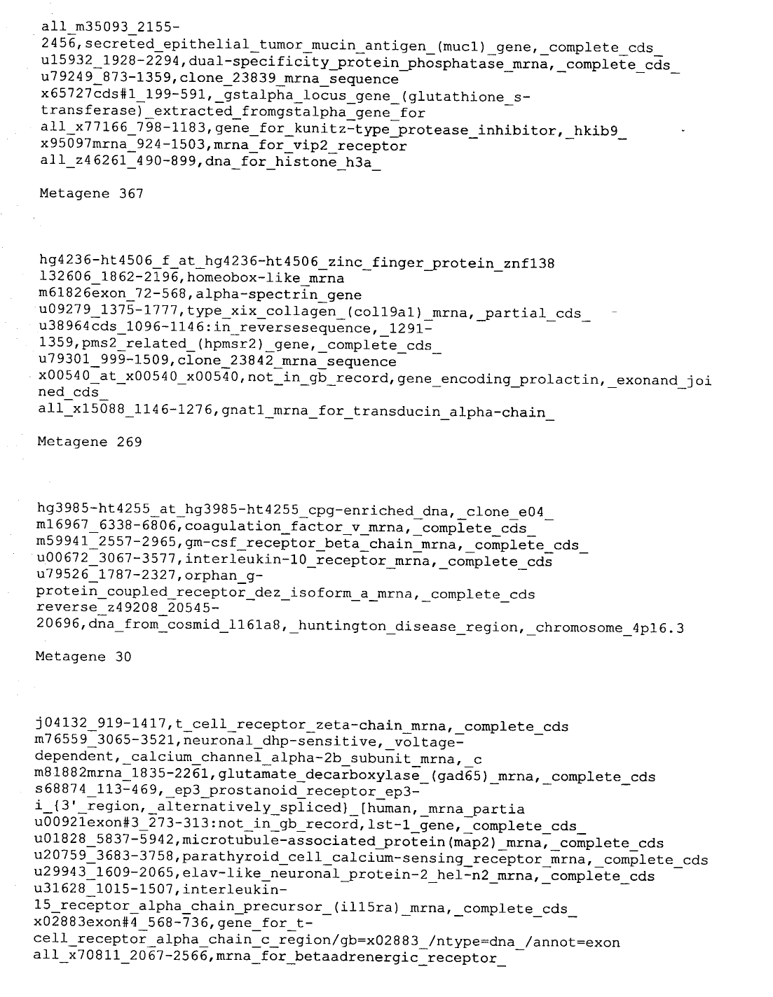

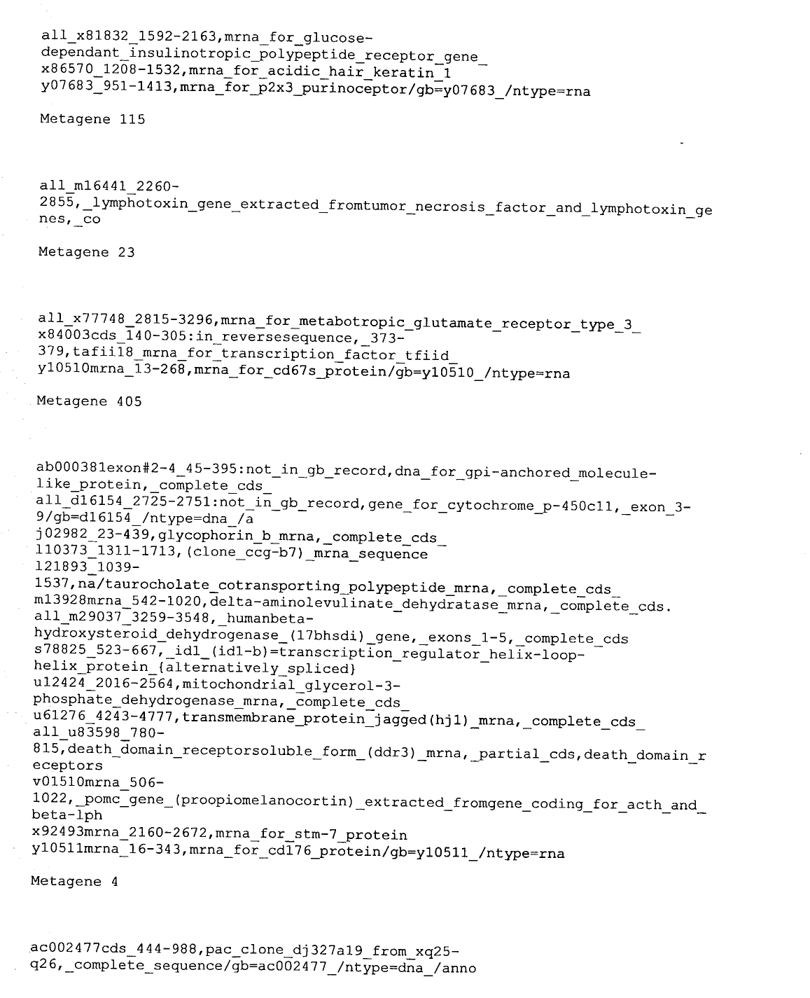

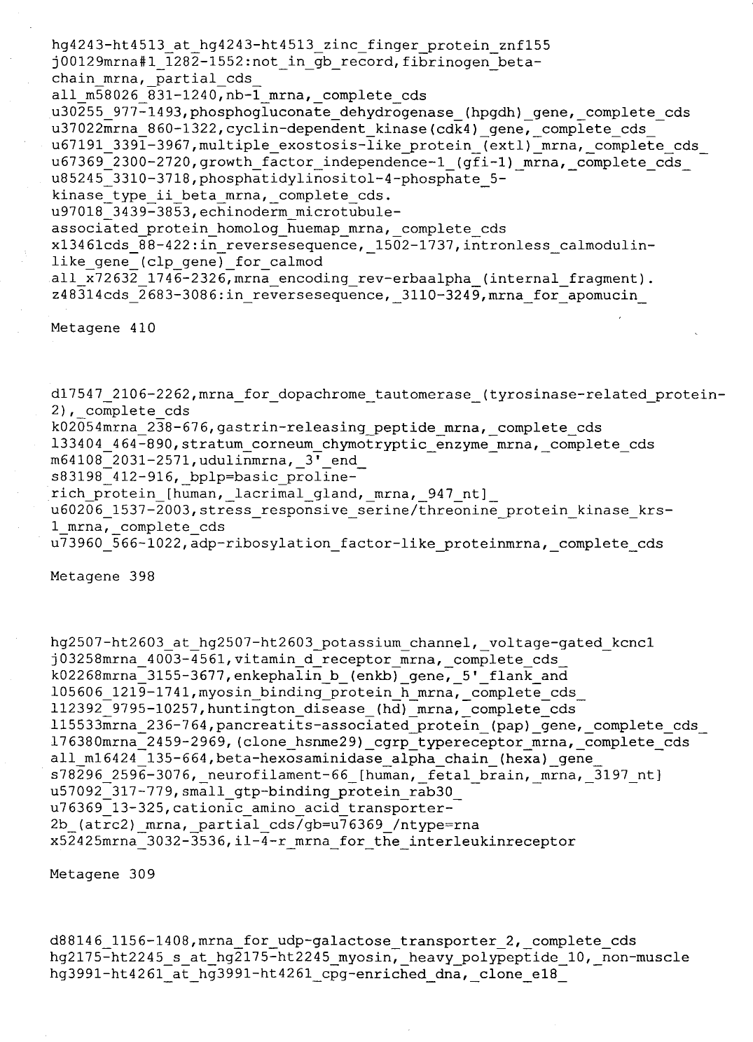

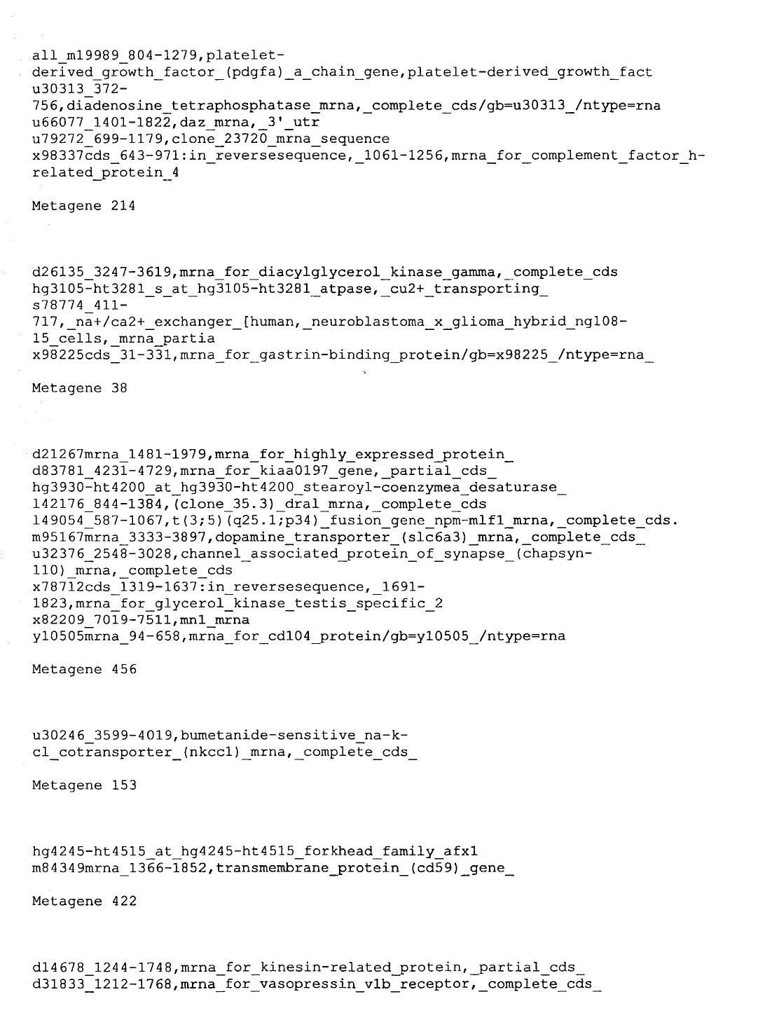

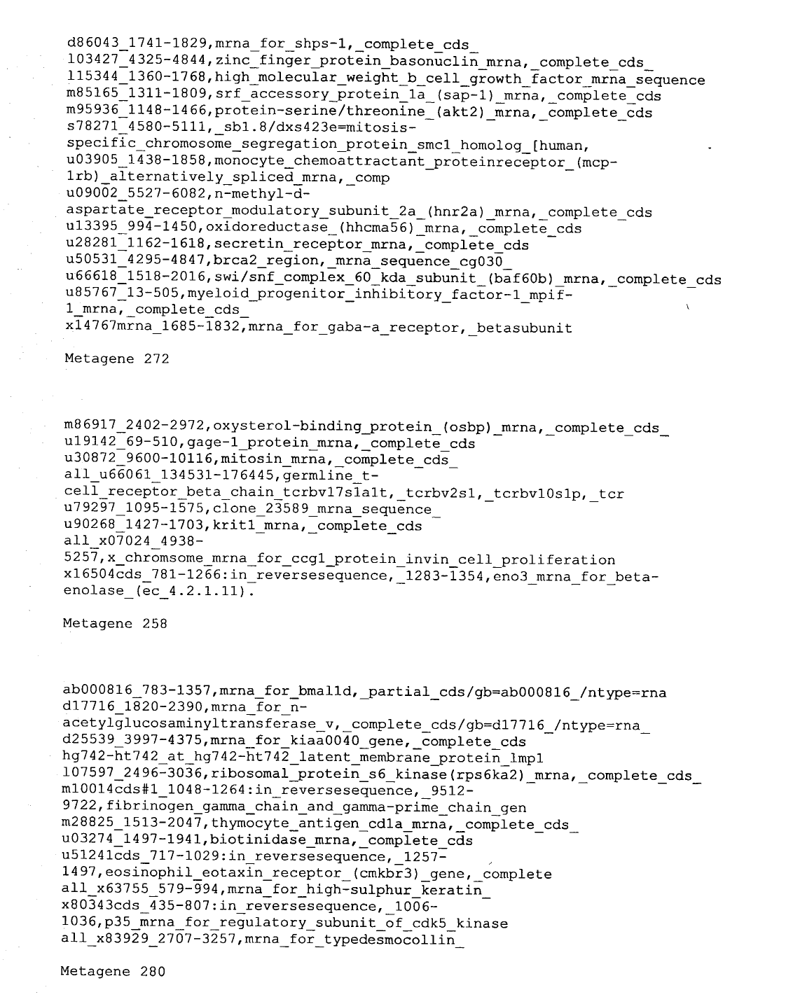

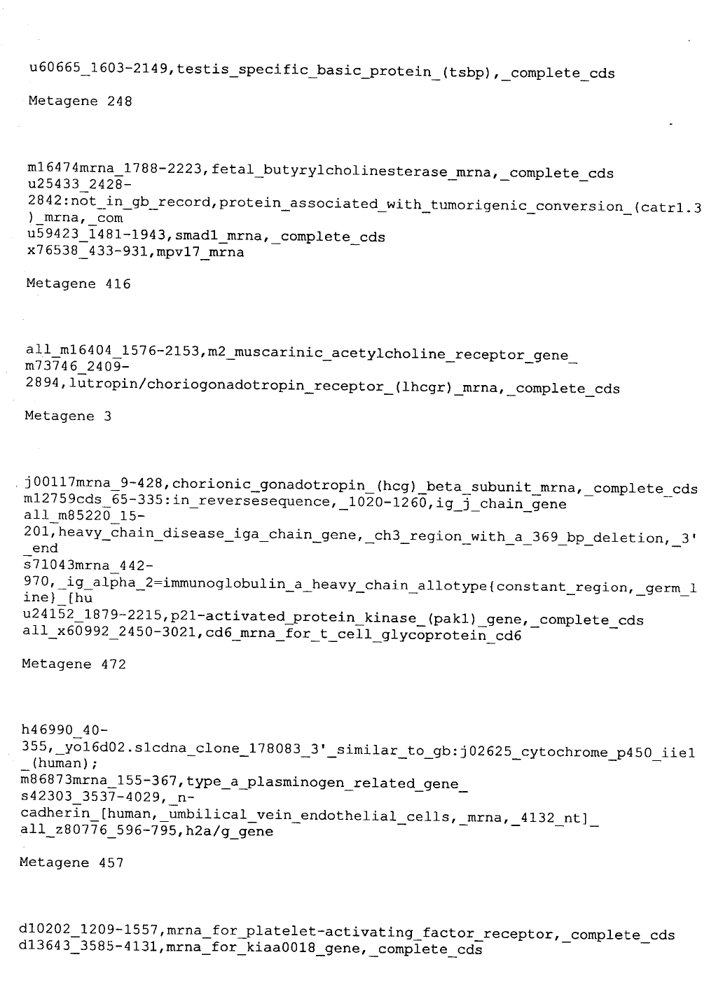

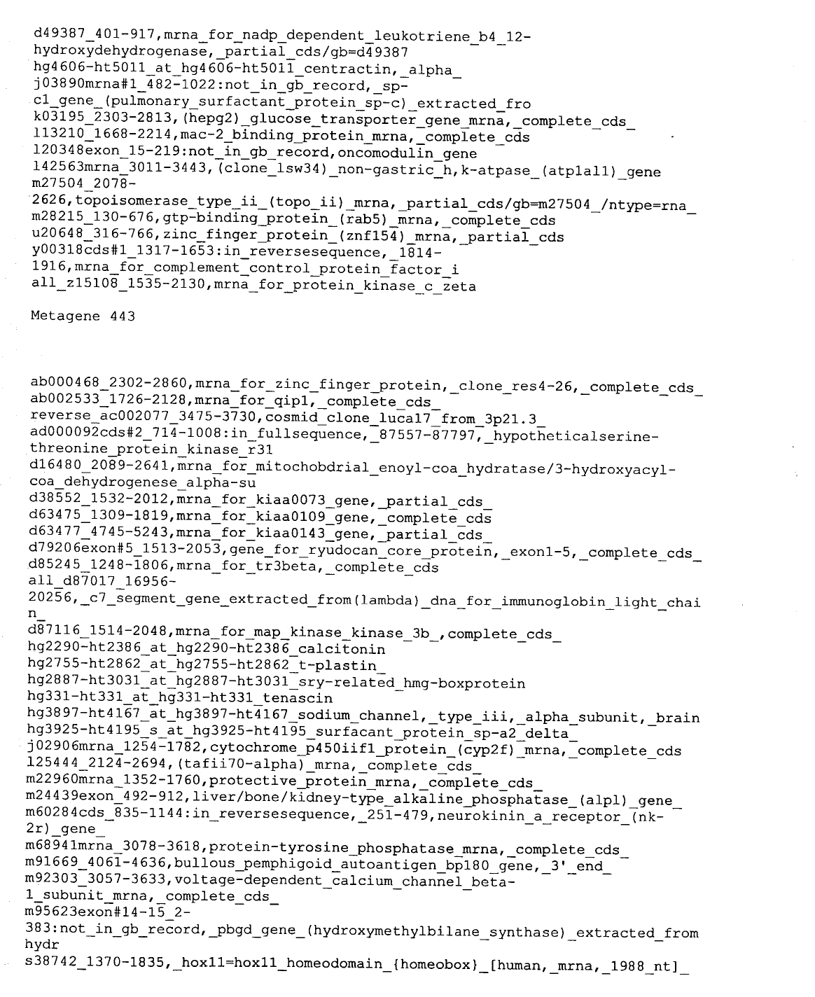

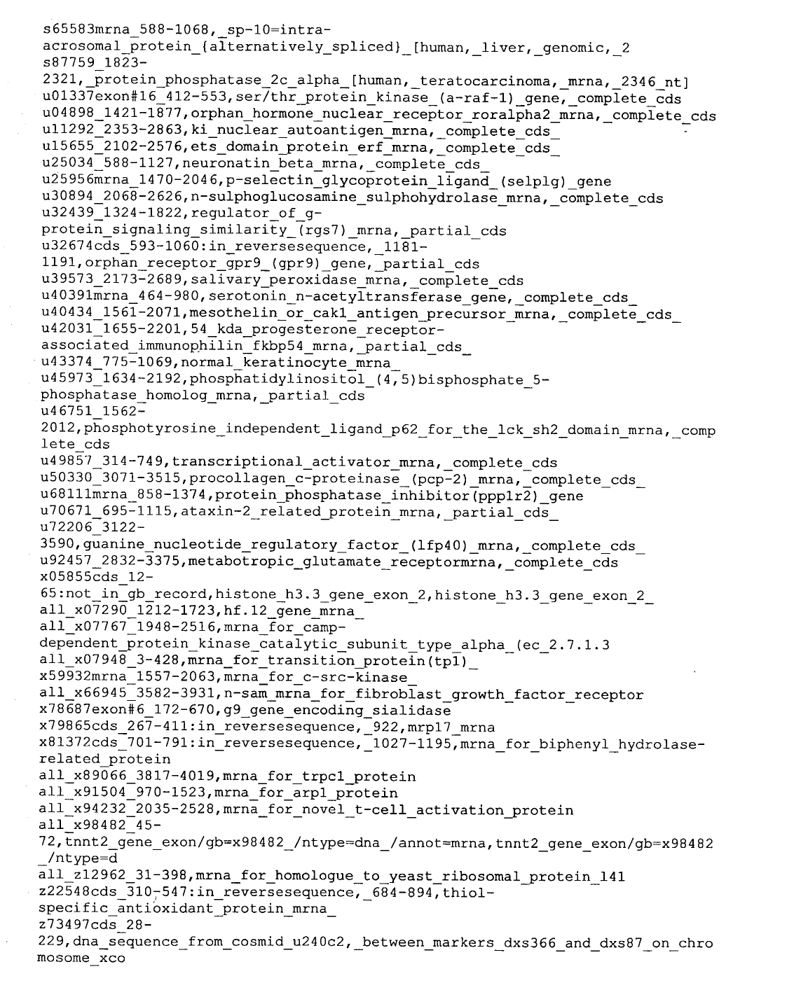

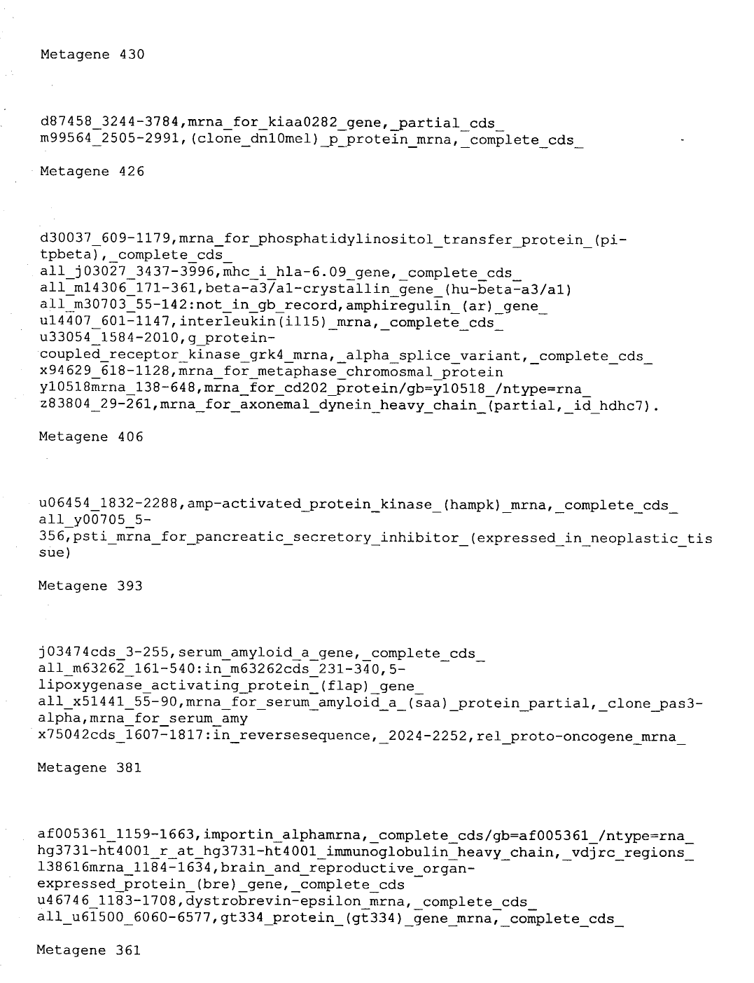

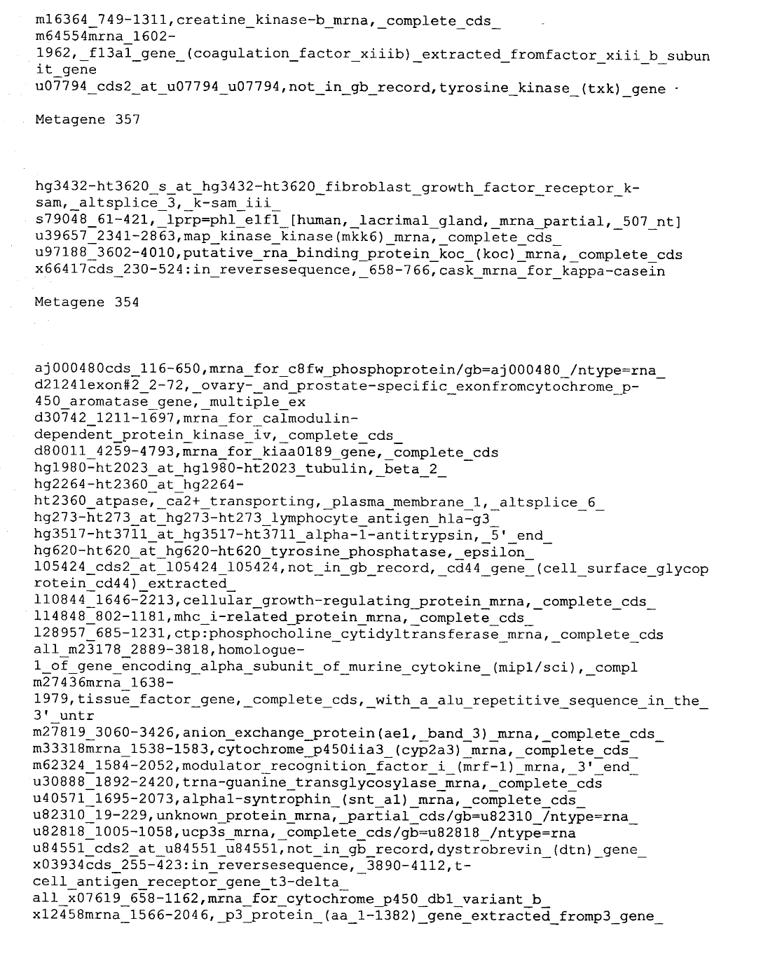

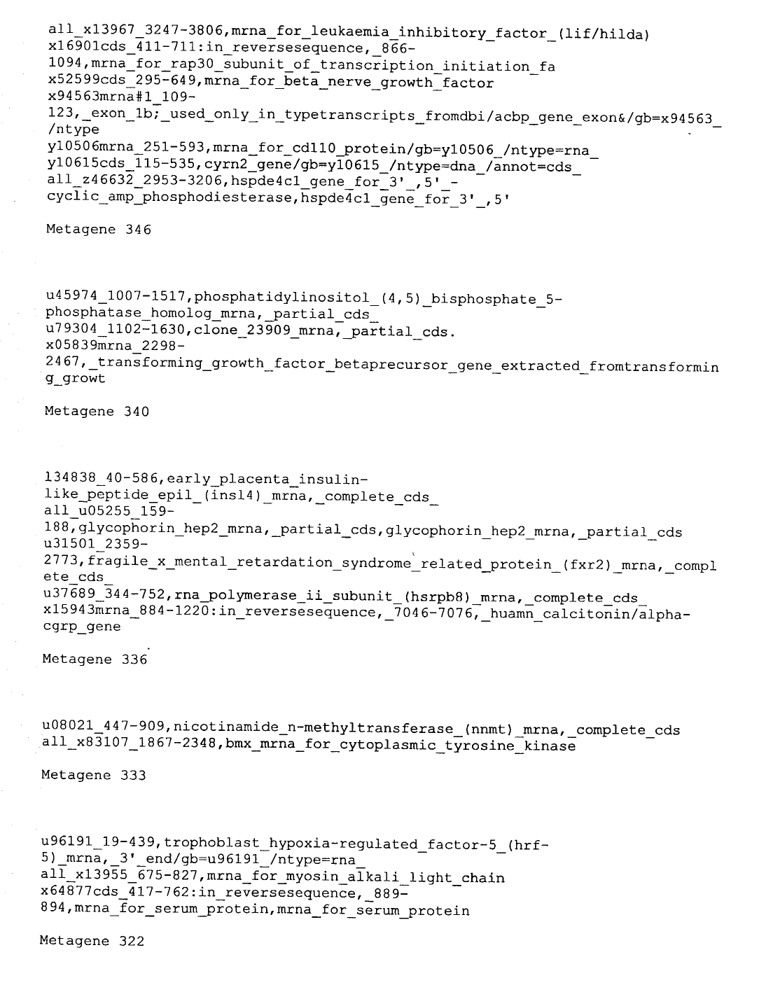

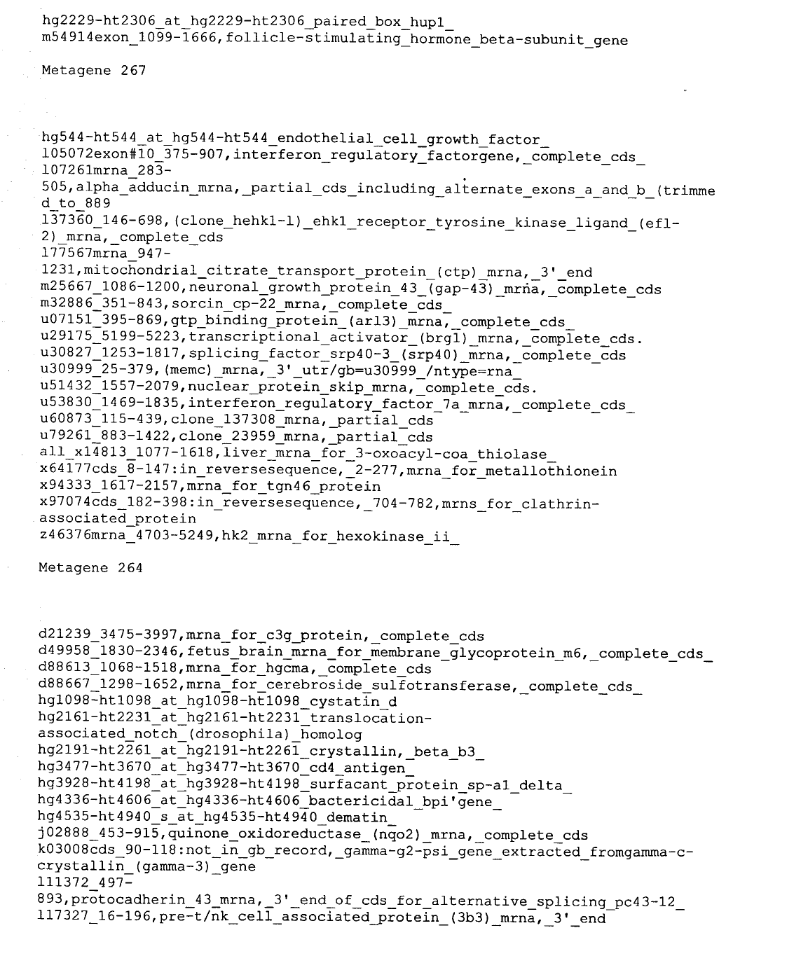

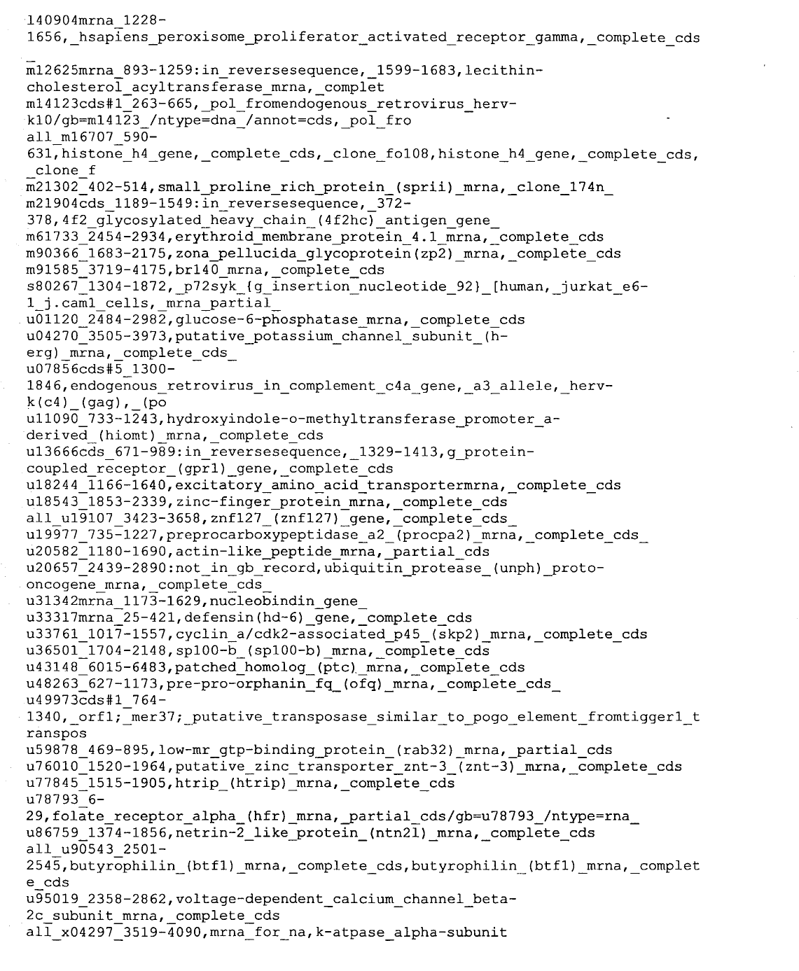

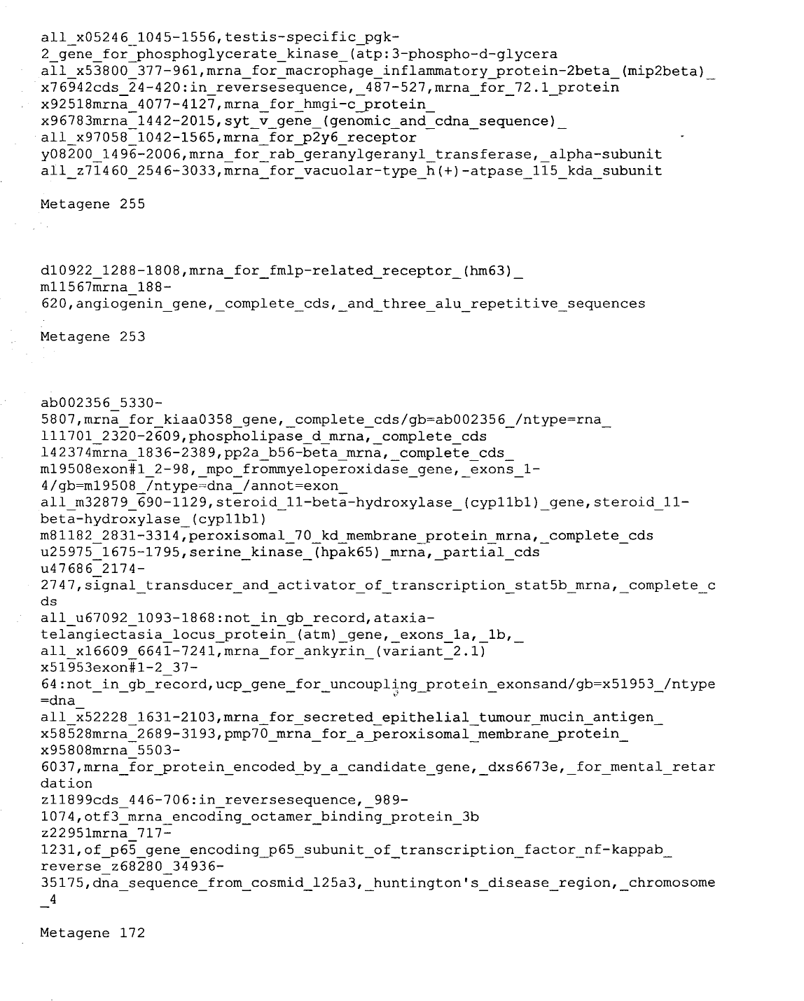

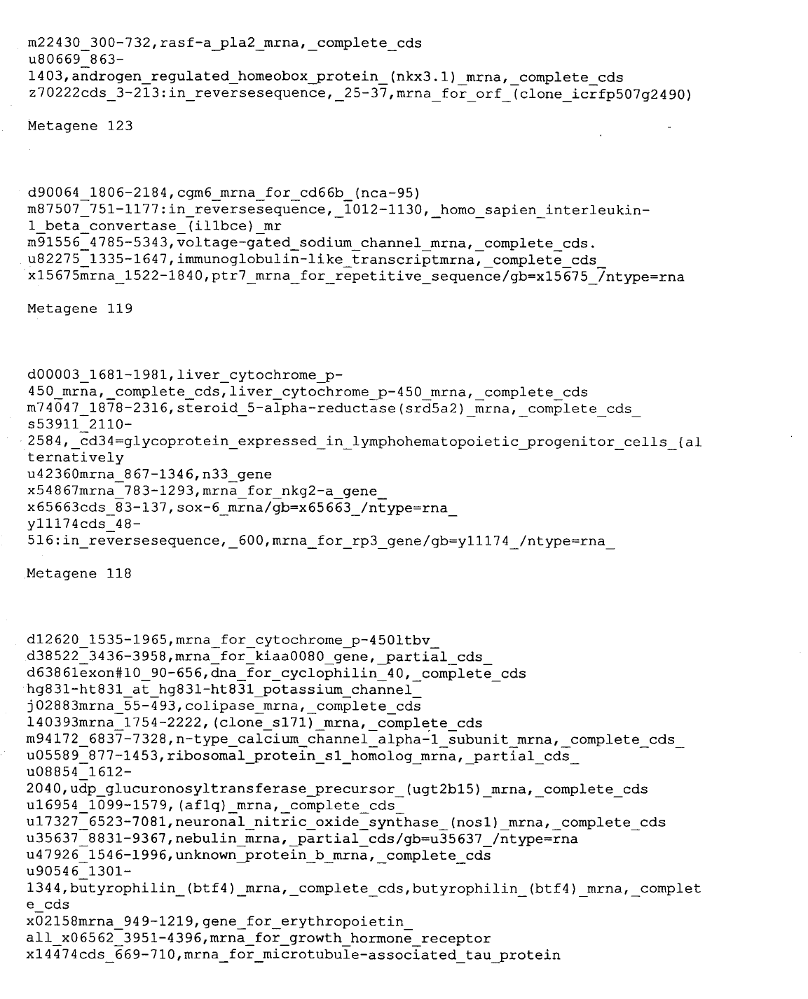

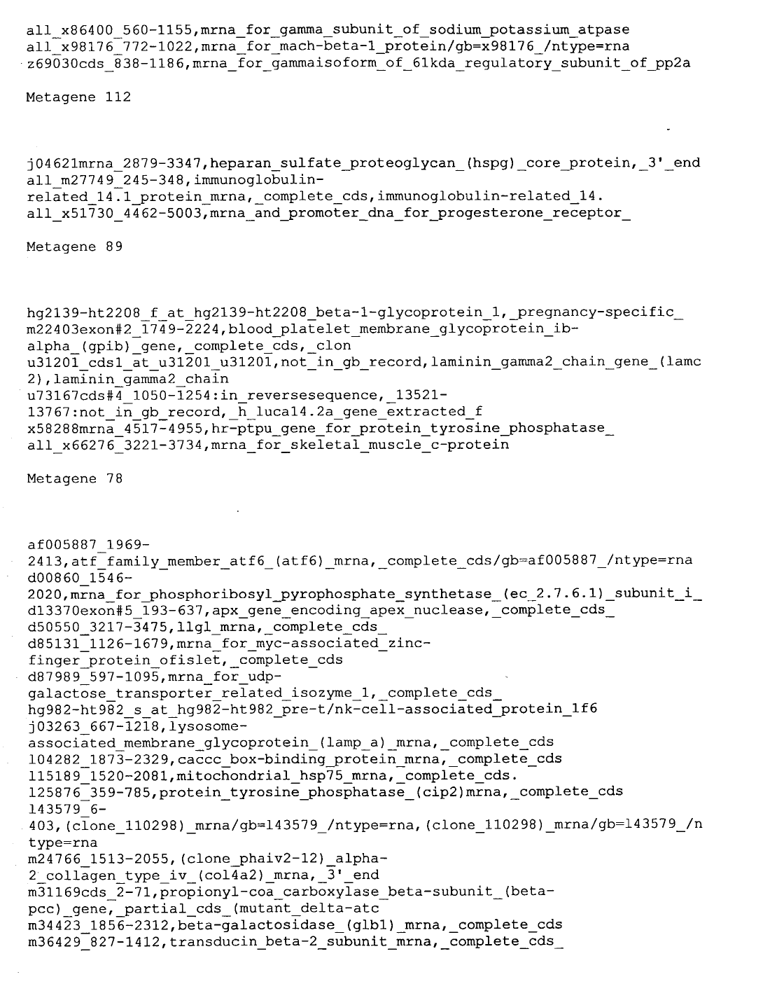

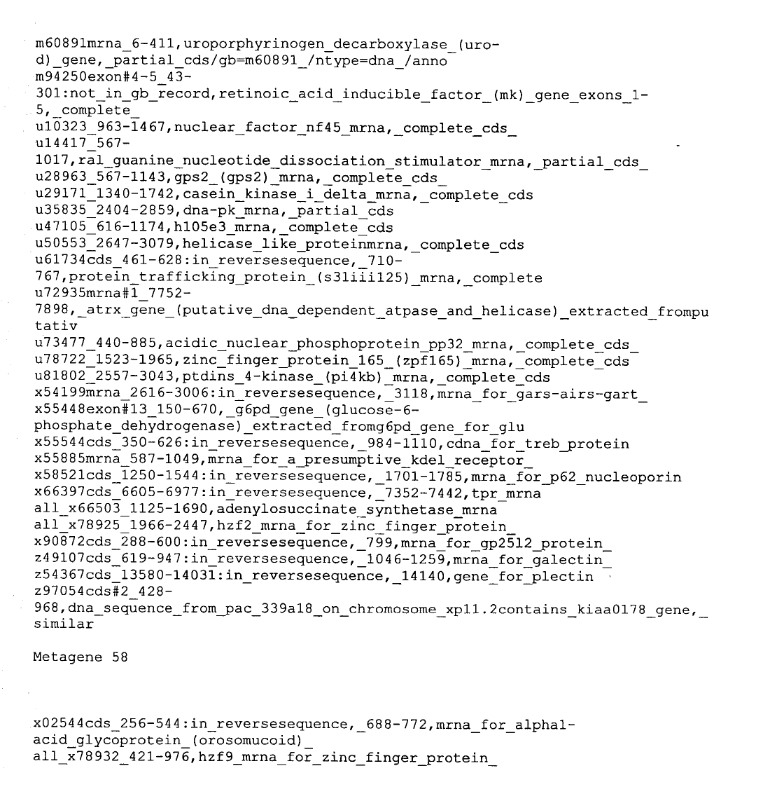

In the analysis of the ER data in this disclosure, the original data was developed using Affymetrix arrays with 7129 sequences, of which 7070 were used (following removal of Affymetrix controls from the data). The expression estimates used were log2 values of the signal intensity measures computed using the dChip software for post-processing Affymetrix output data (See Li, C. and Wong, W. H. Model-based analysis of oligonucleotide arrays: Expression index computation and outlier detection. Proc. Natl. Acad. Sci., 98, 31-36 (2001), and the software site http://www.biostat.harvard.edu/complab/dchip/). With a target of 500 clusters, the xcluster software implementing the correlation-based k_means clustering produced p=491 clusters. The corresponding p metagenes were then evaluated as the dominant singular factors of each of these cluster, as referenced above. See FIGS. 4-5 that provide tables detailing the 491 metagenes.

The data comprised 40 training samples and 9 validation cases. Among the latter, 3 were initial training samples that presented conflicting laboratory tests of the ER protein levels, so casting into question their actual ER status; these were therefore placed in the validation sample to be predicted, along with an initial 6 validation cases selected at random. These three cases are numbers 14, 31 and 33. The color coding in the graphs is based on the first laboratory test (immunohistochemistry). Additional samples of interest are cases 7, 8 and 11, cases for which the DNA microarray hybridizations were of poor quality, with the resulting data exhibiting major patterns of differences relative to the rest.

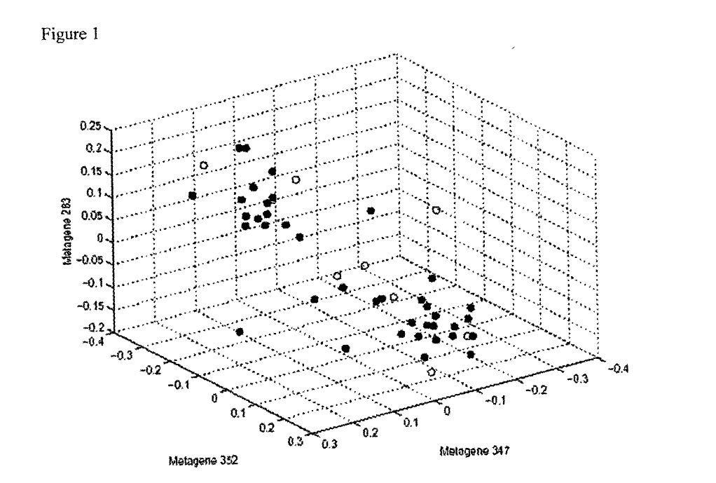

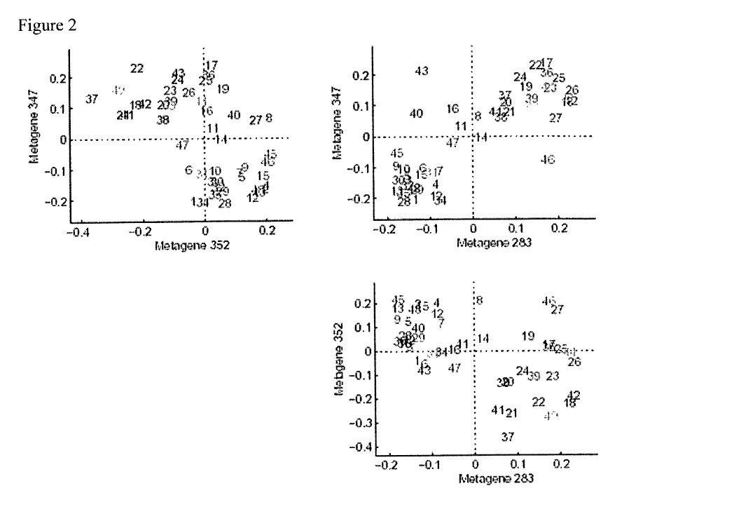

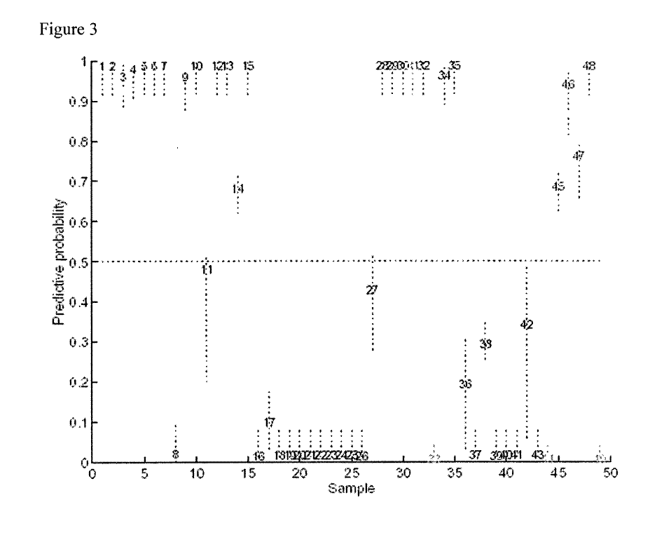

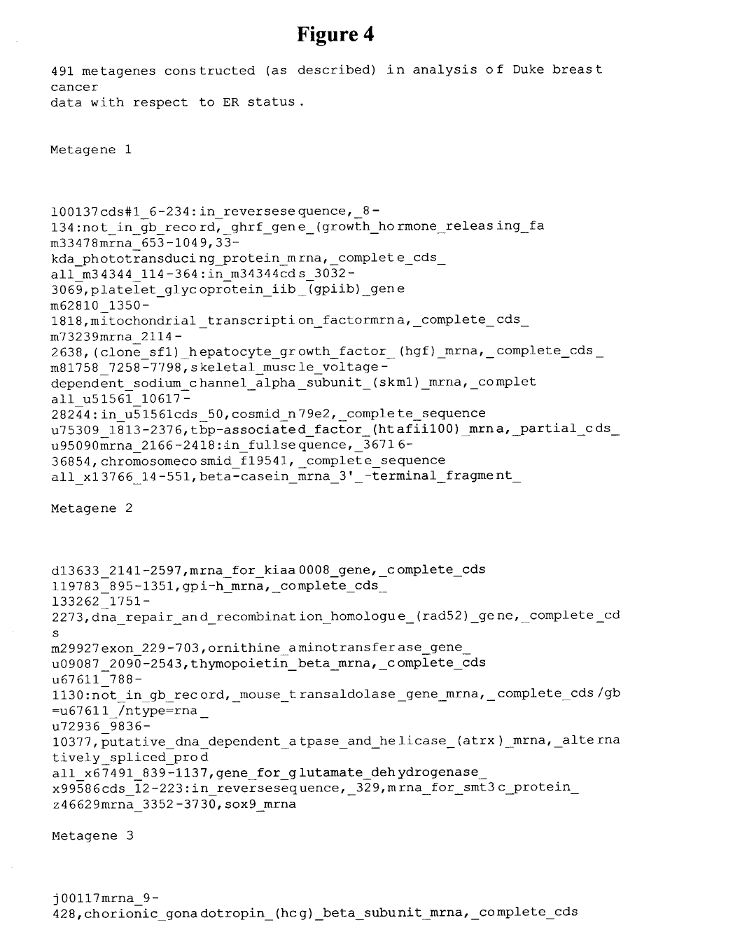

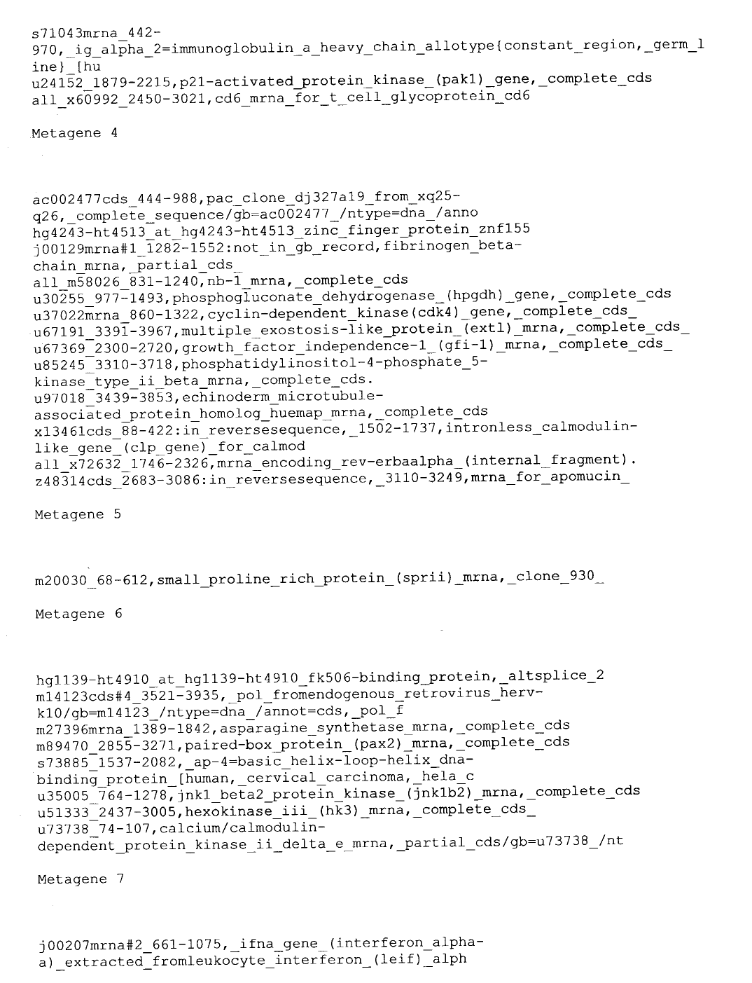

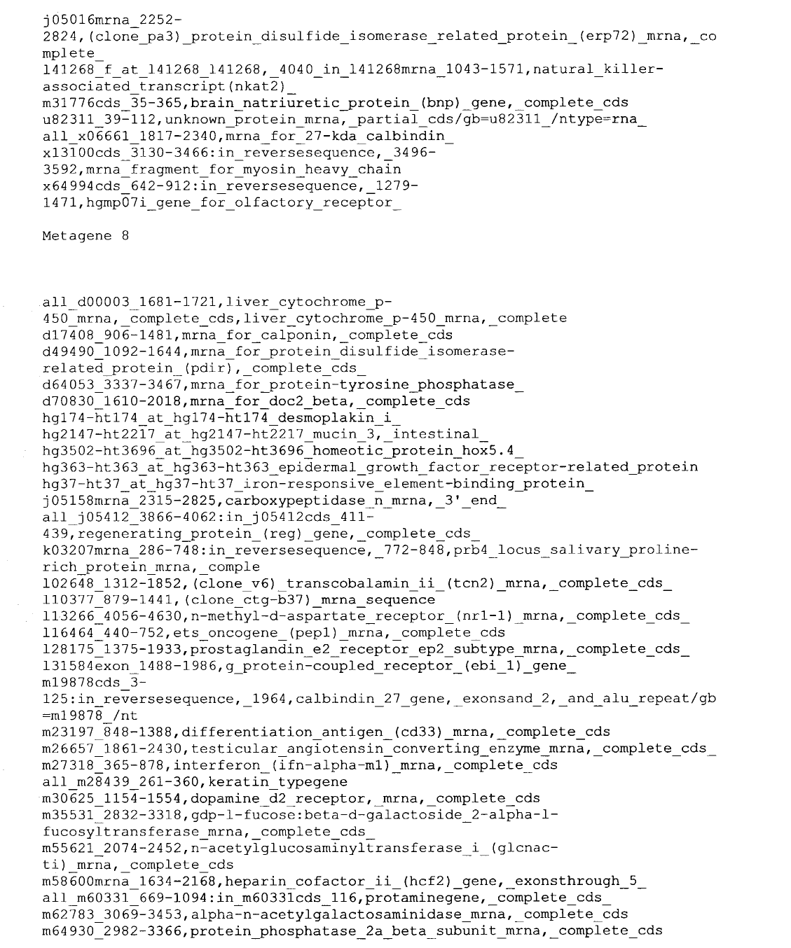

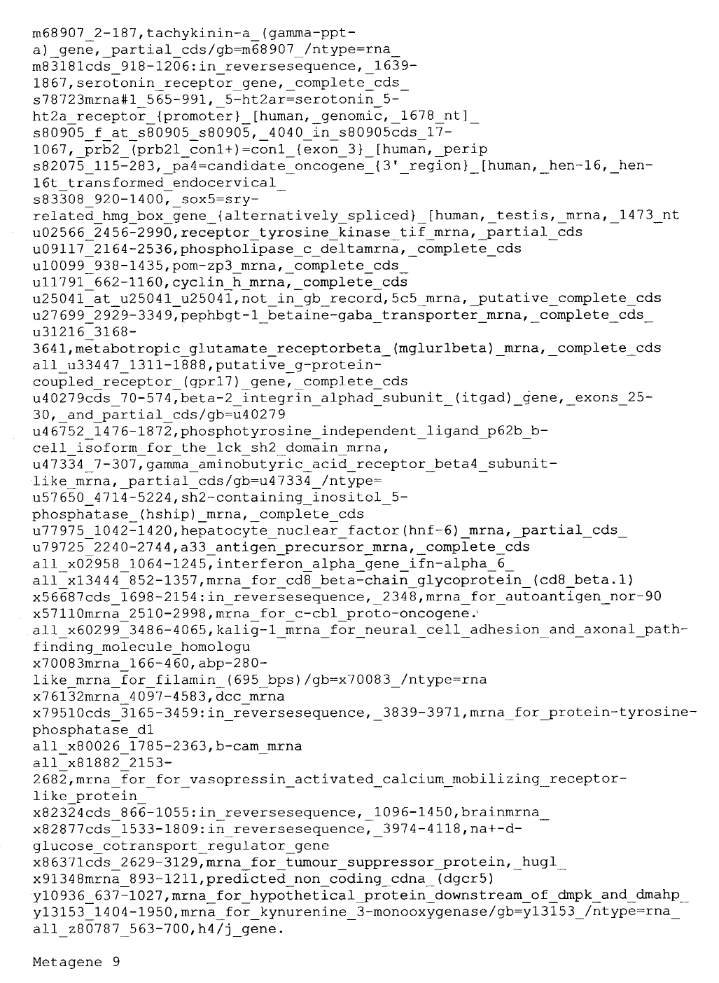

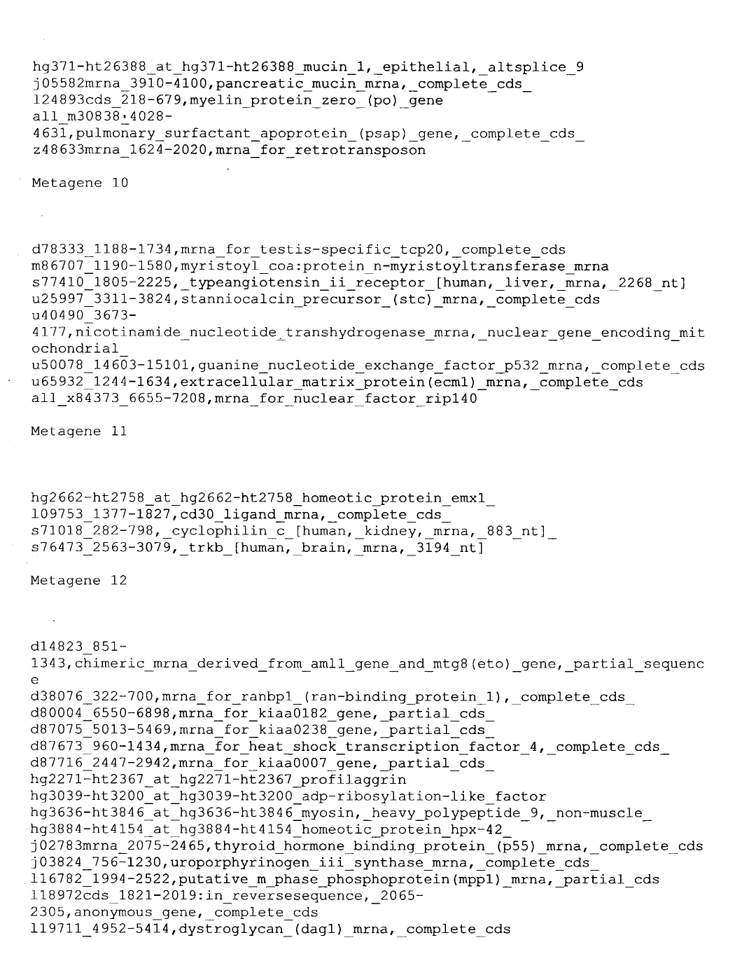

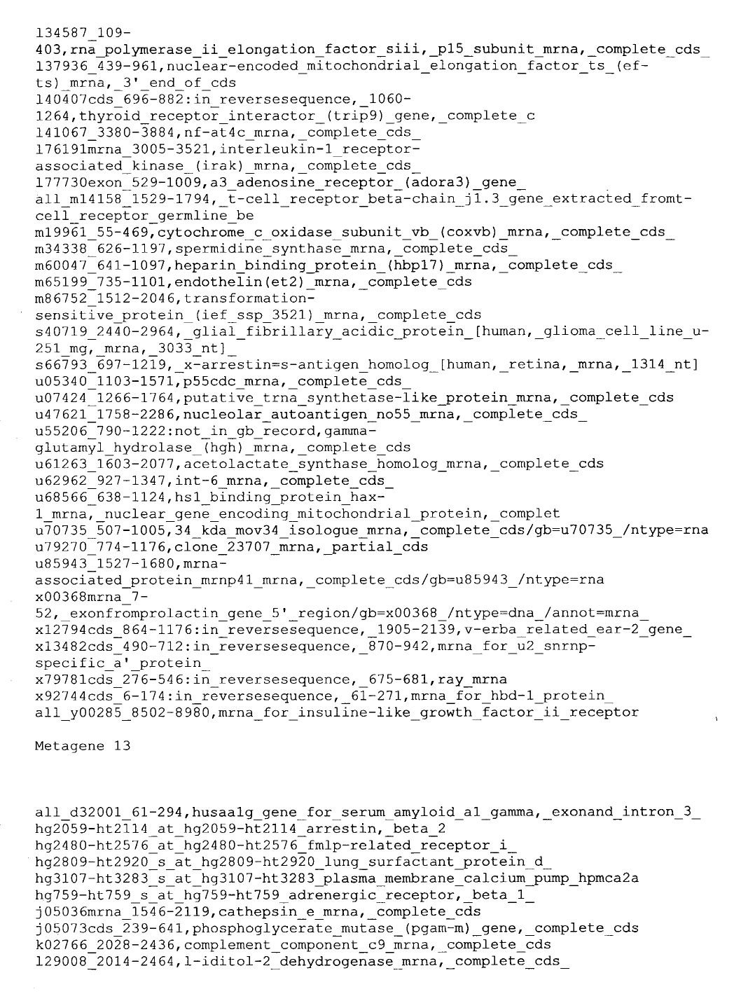

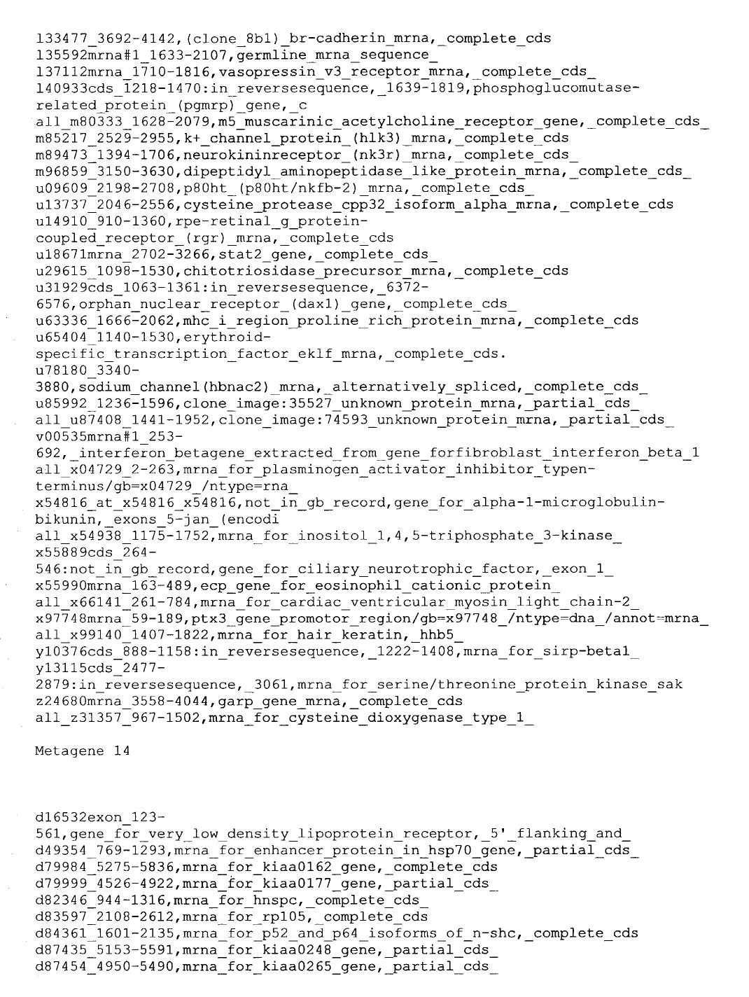

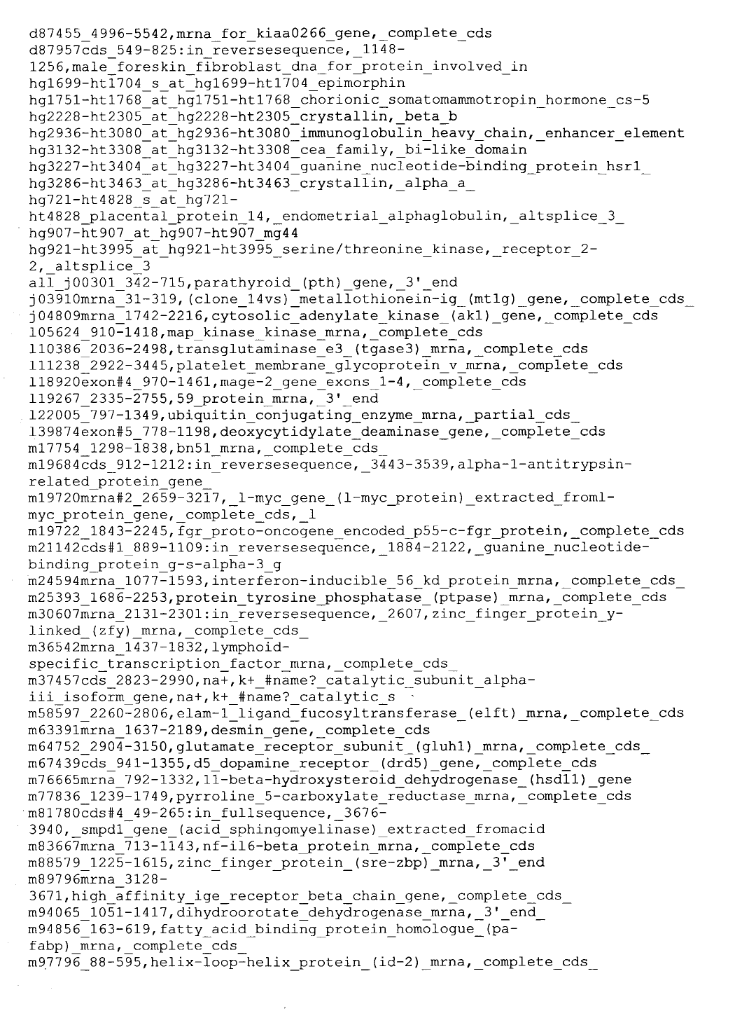

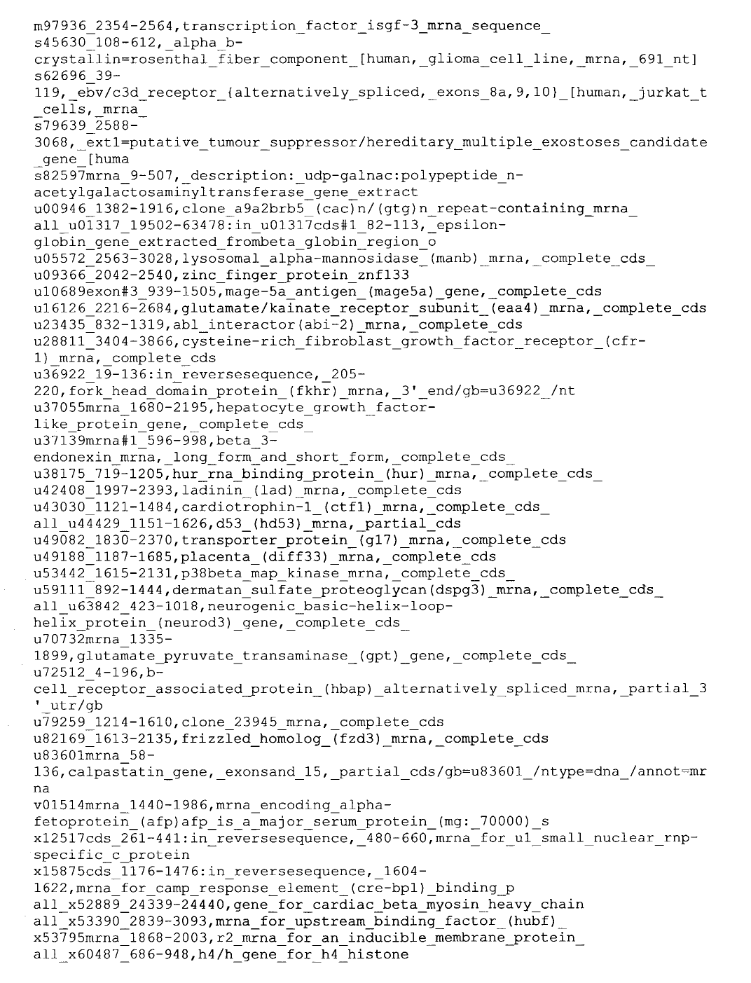

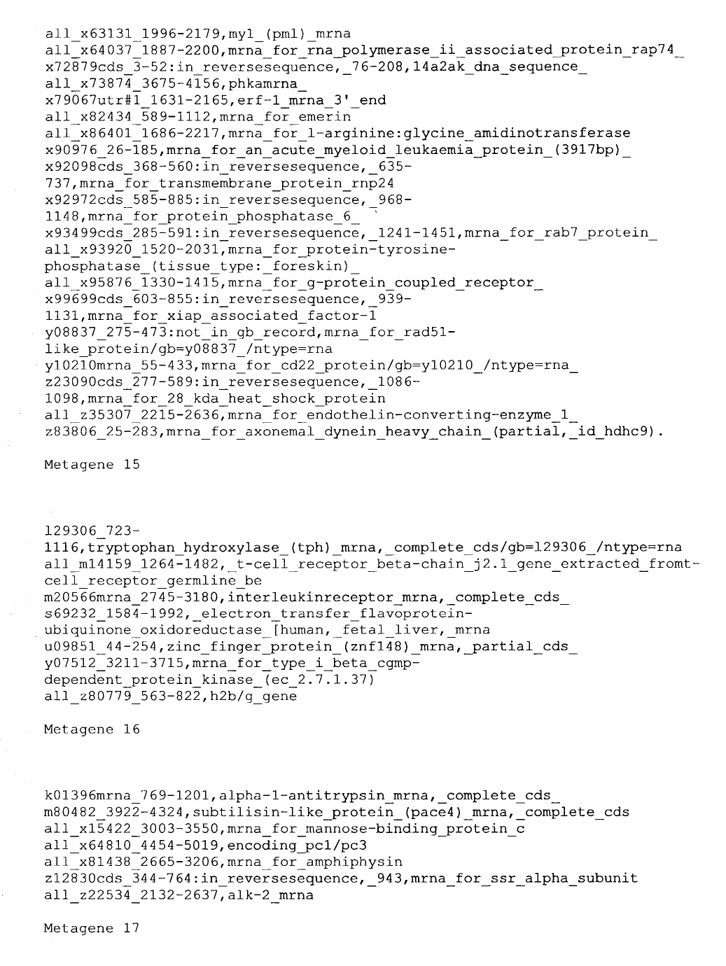

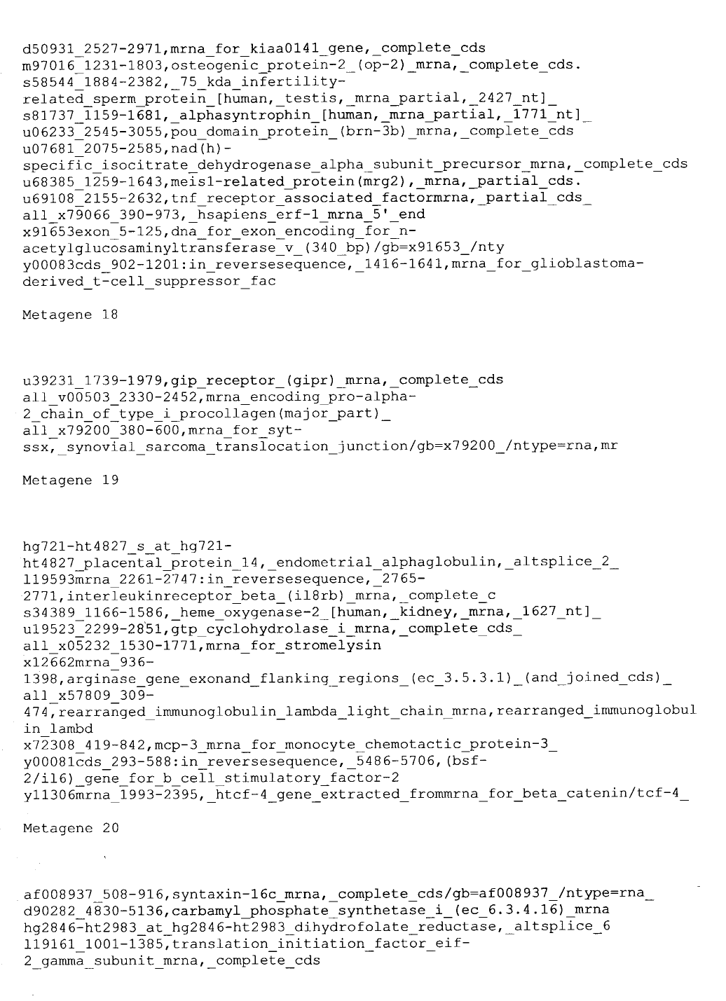

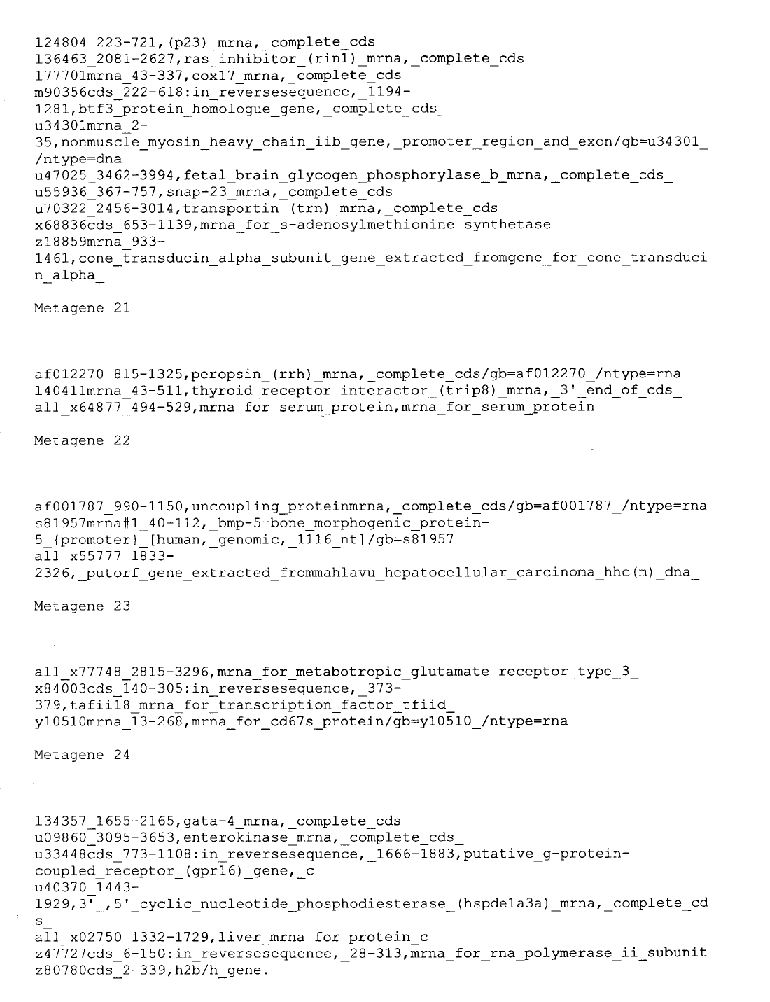

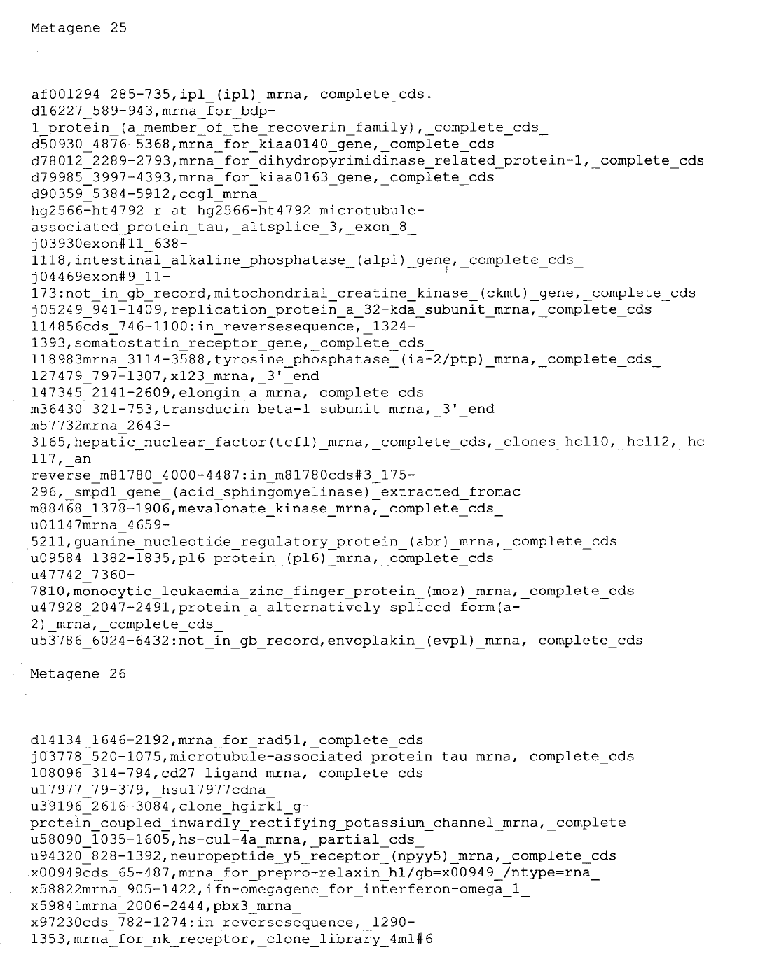

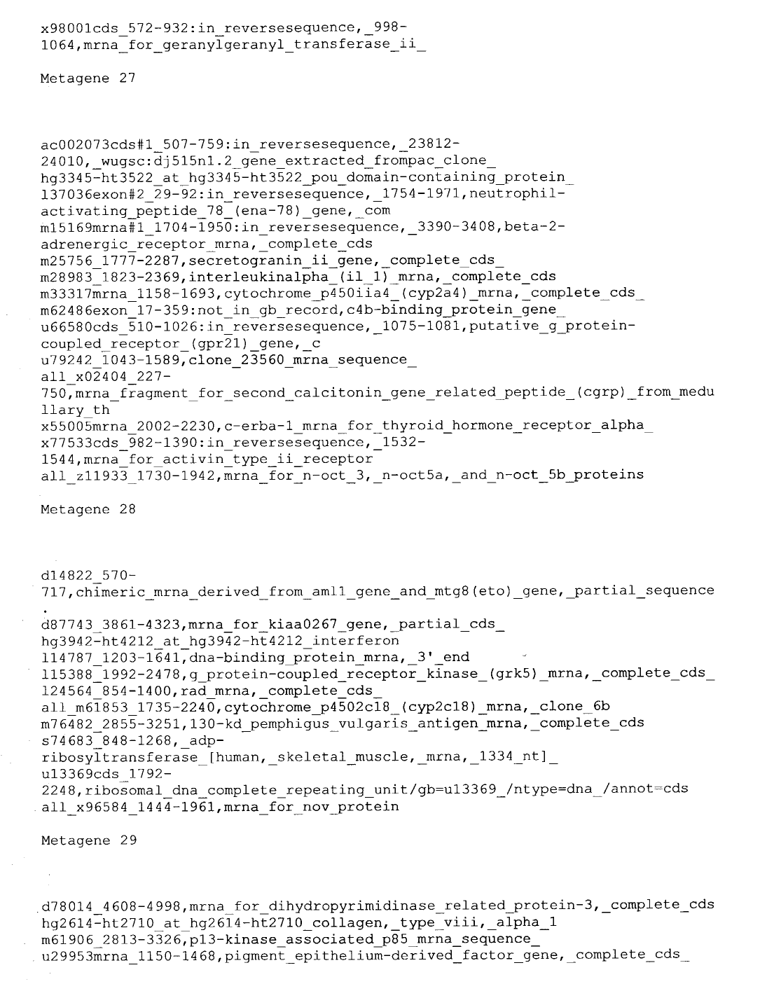

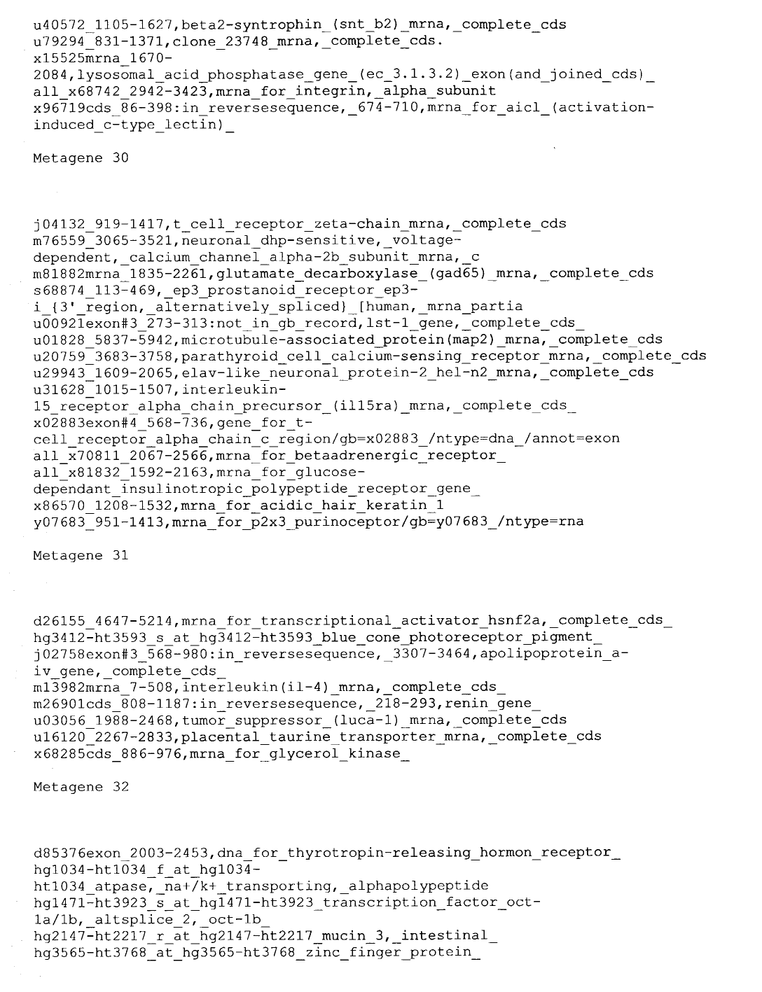

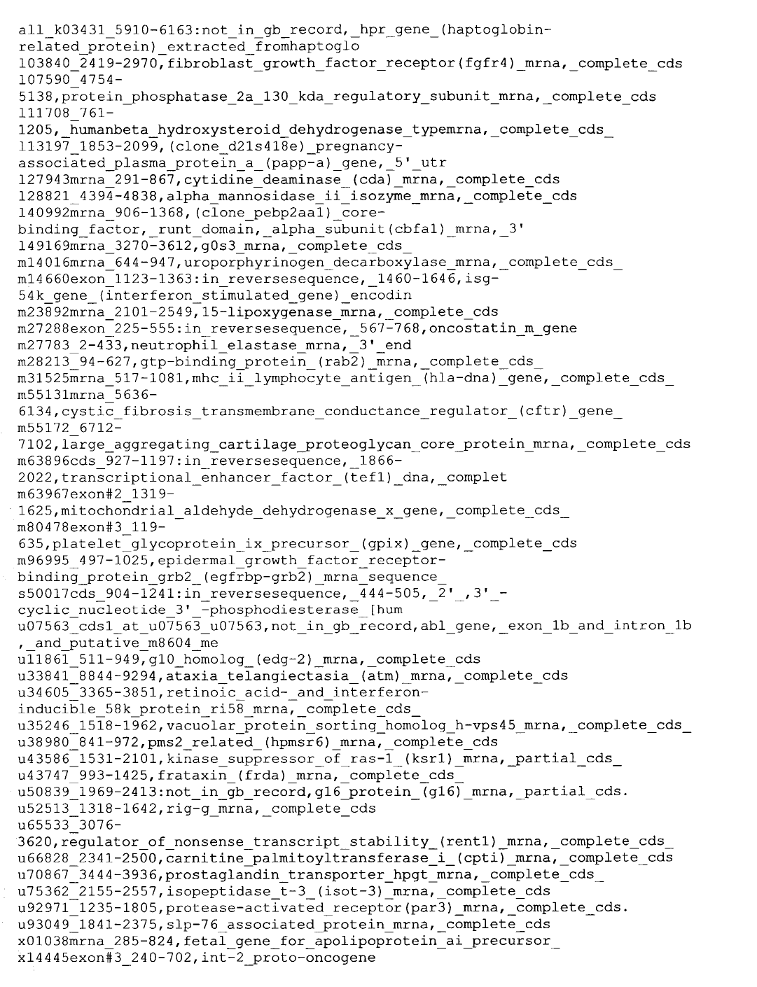

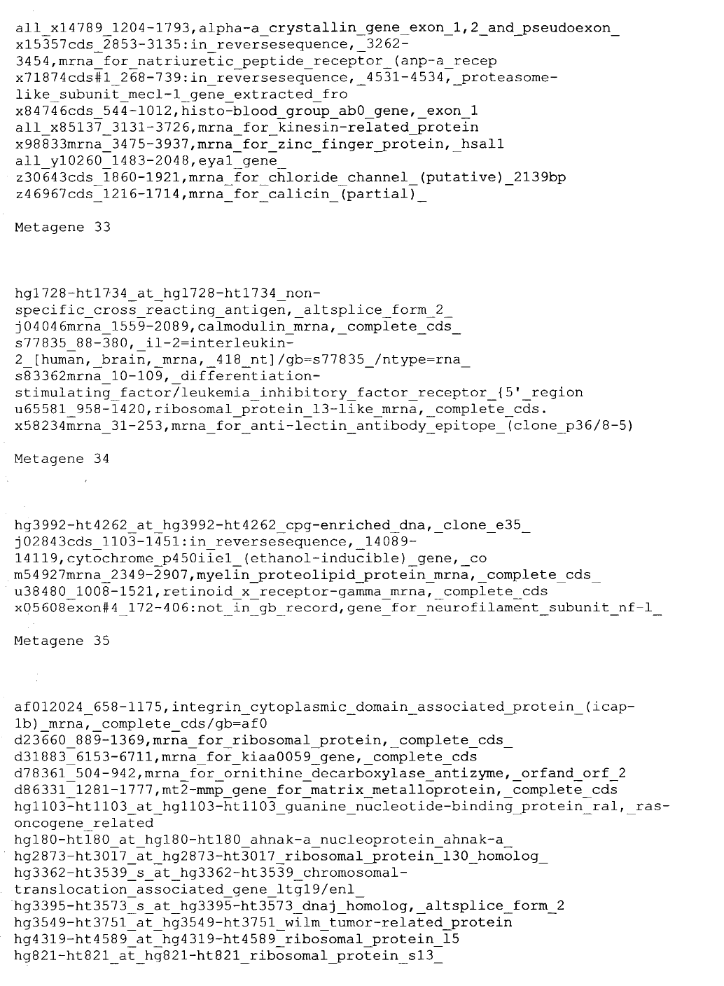

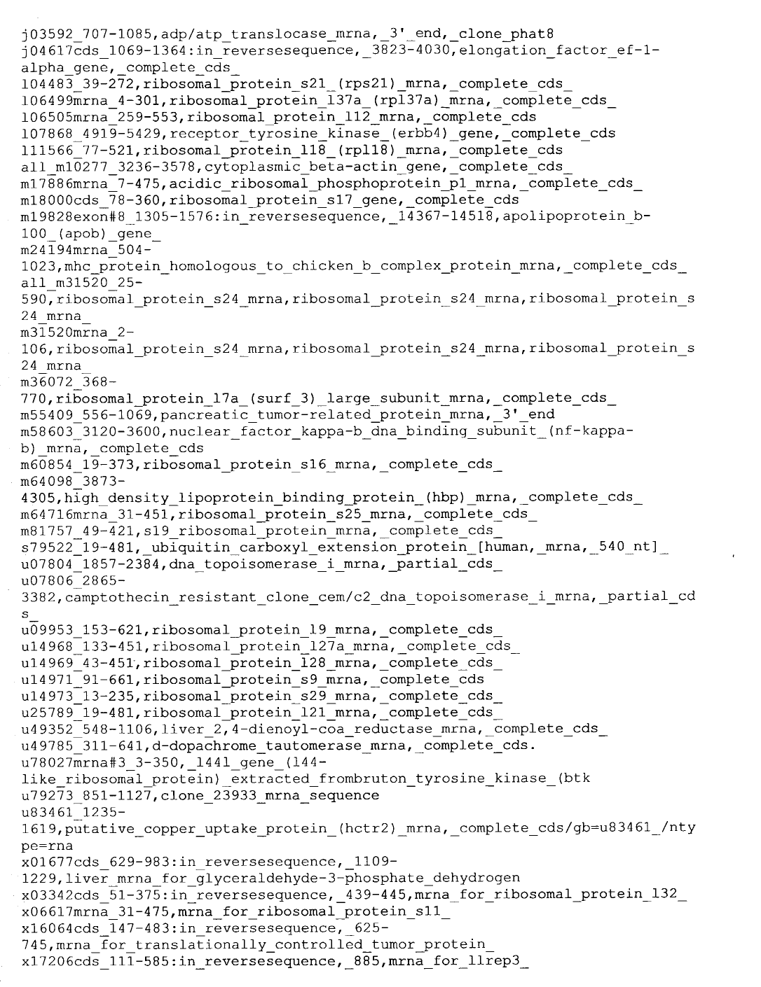

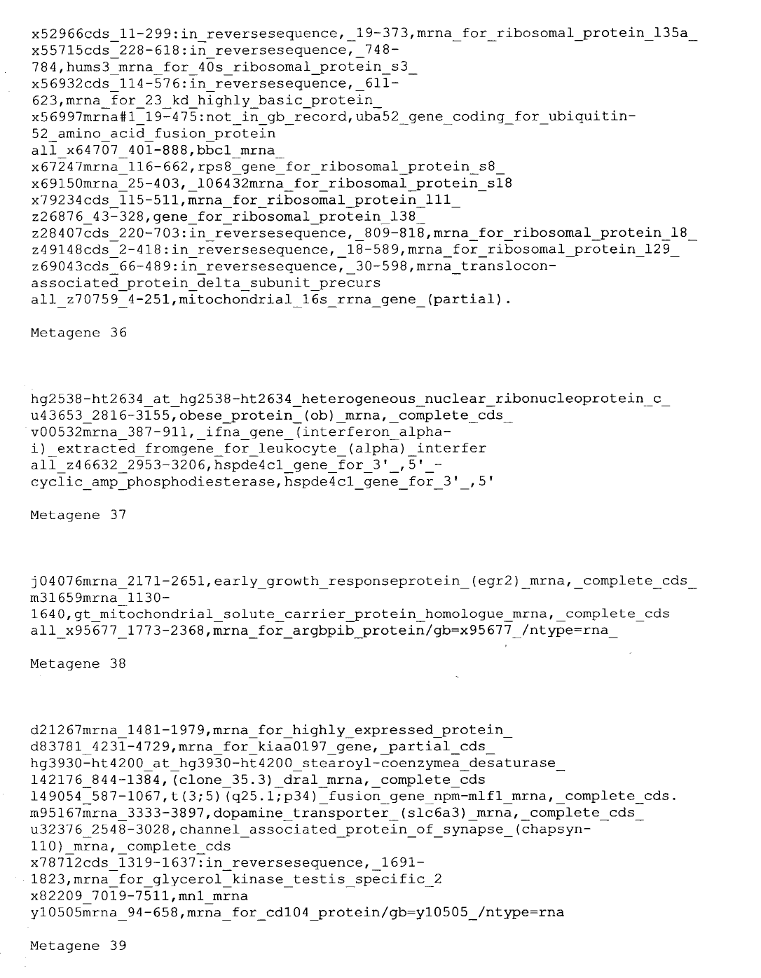

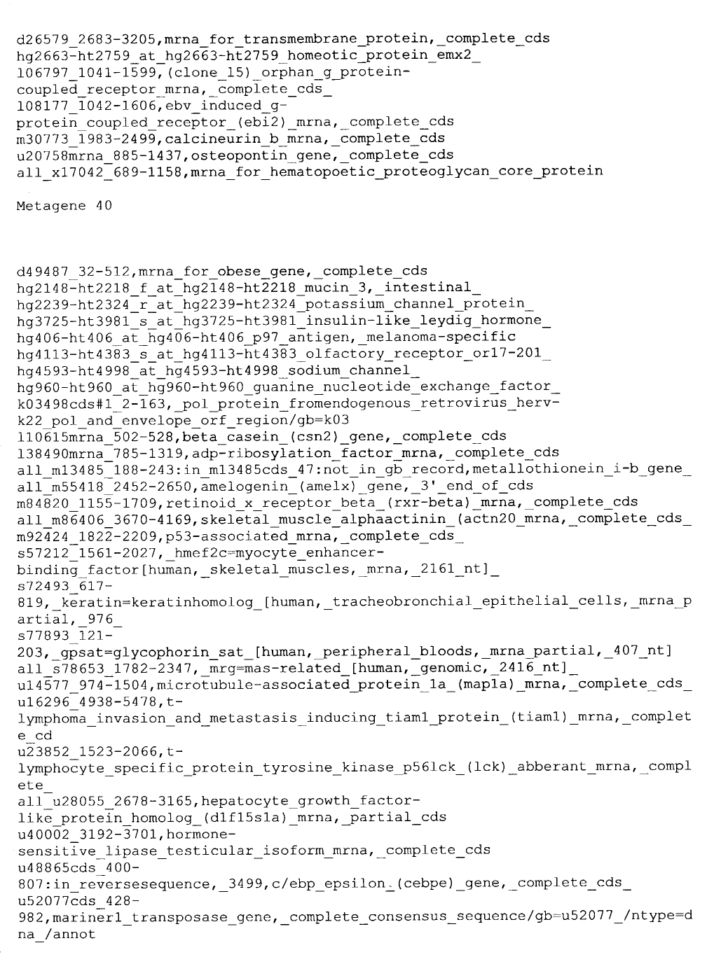

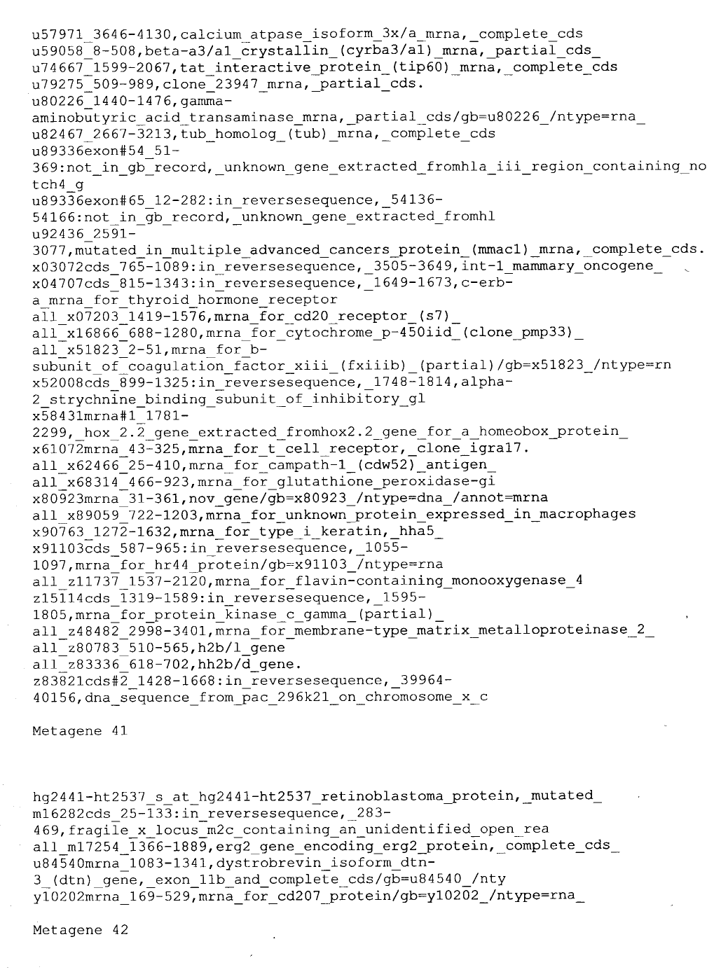

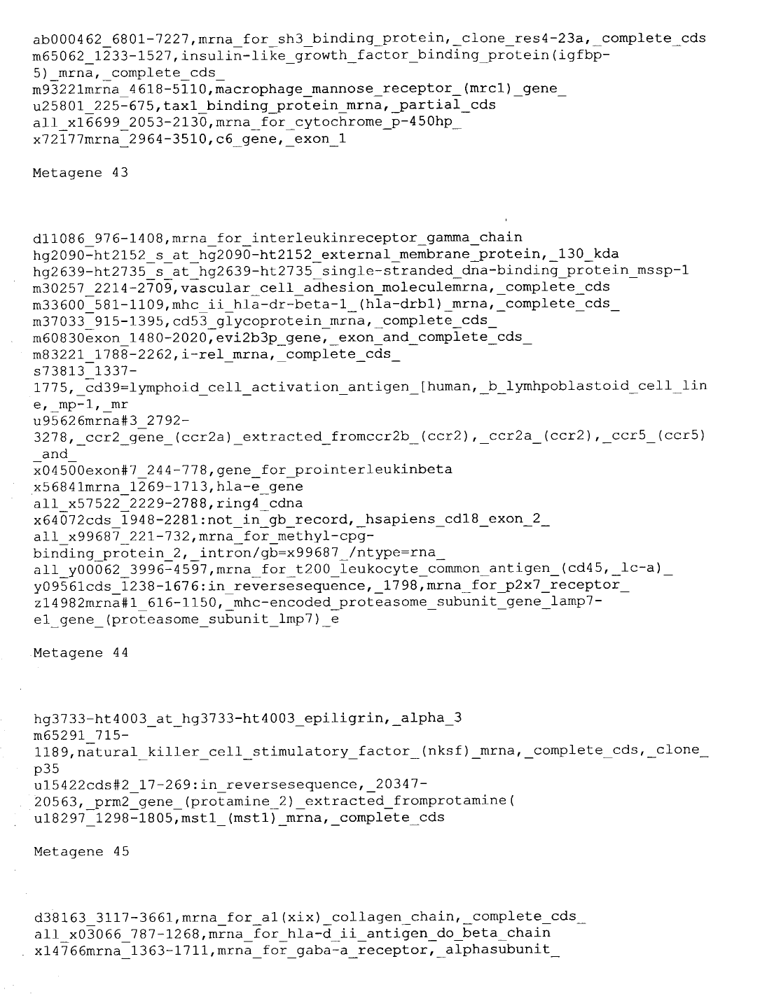

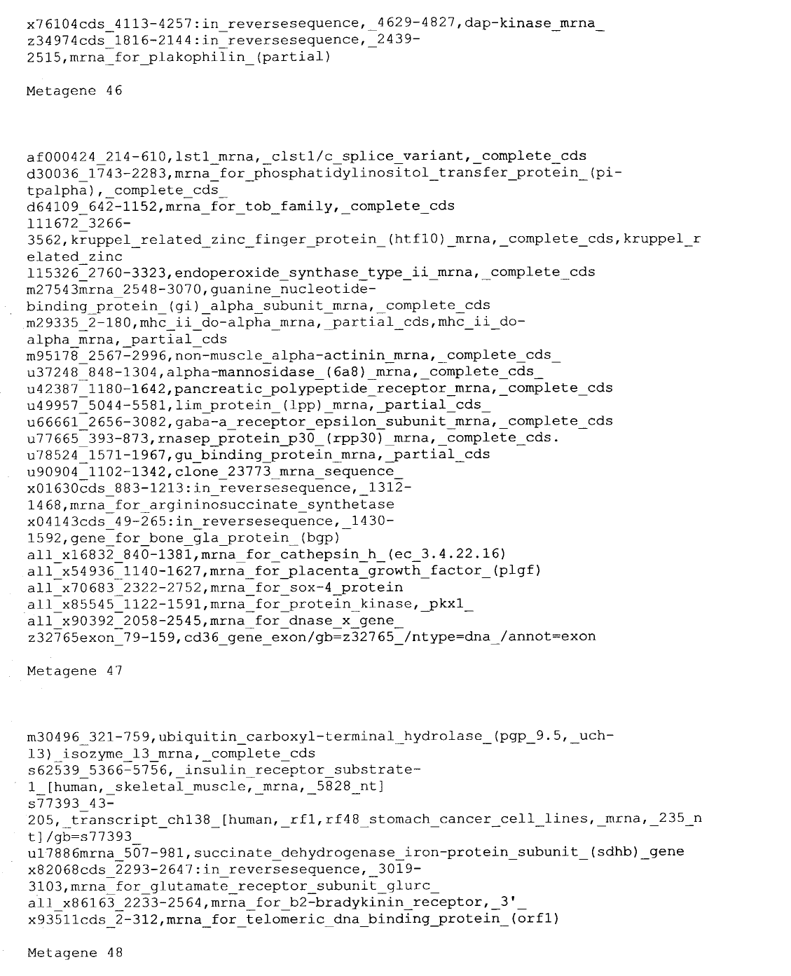

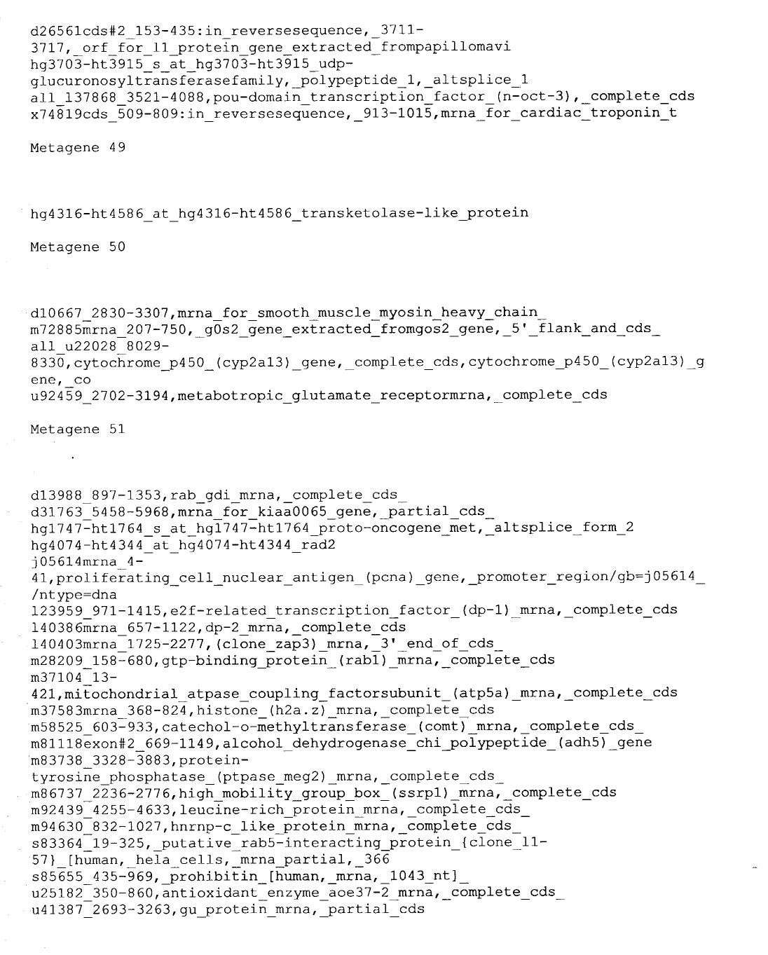

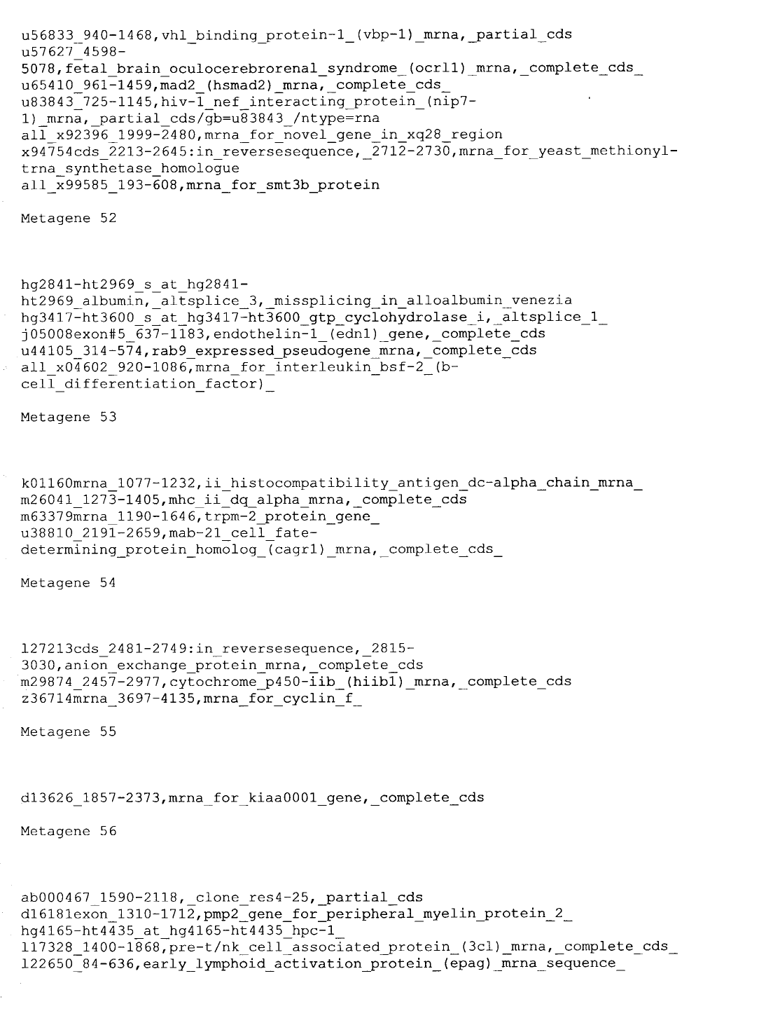

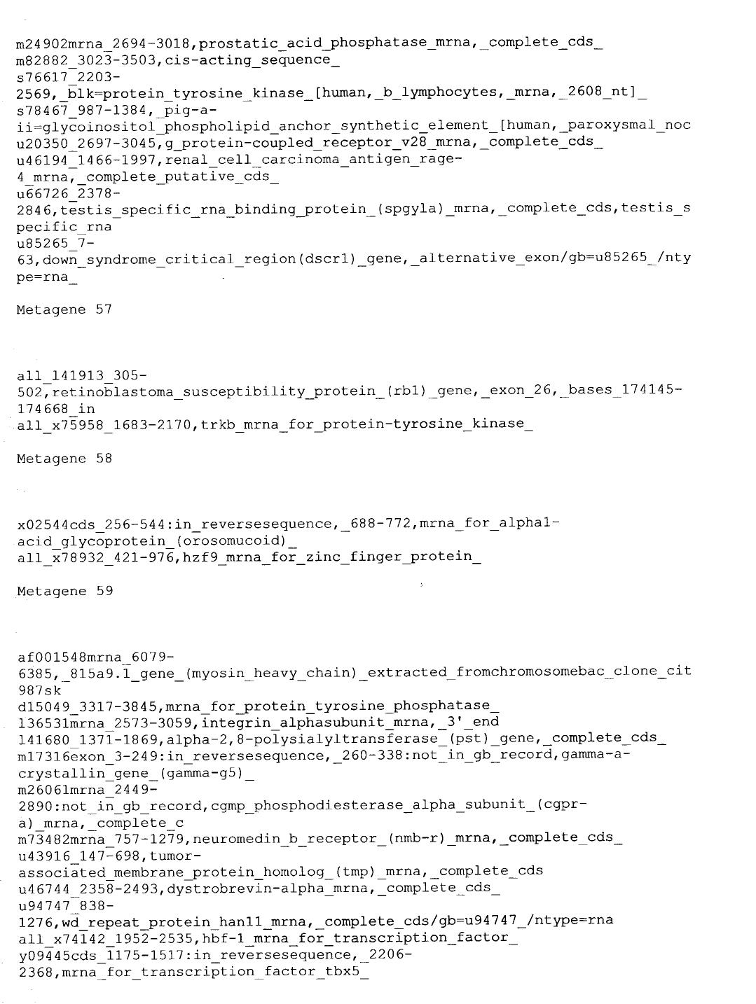

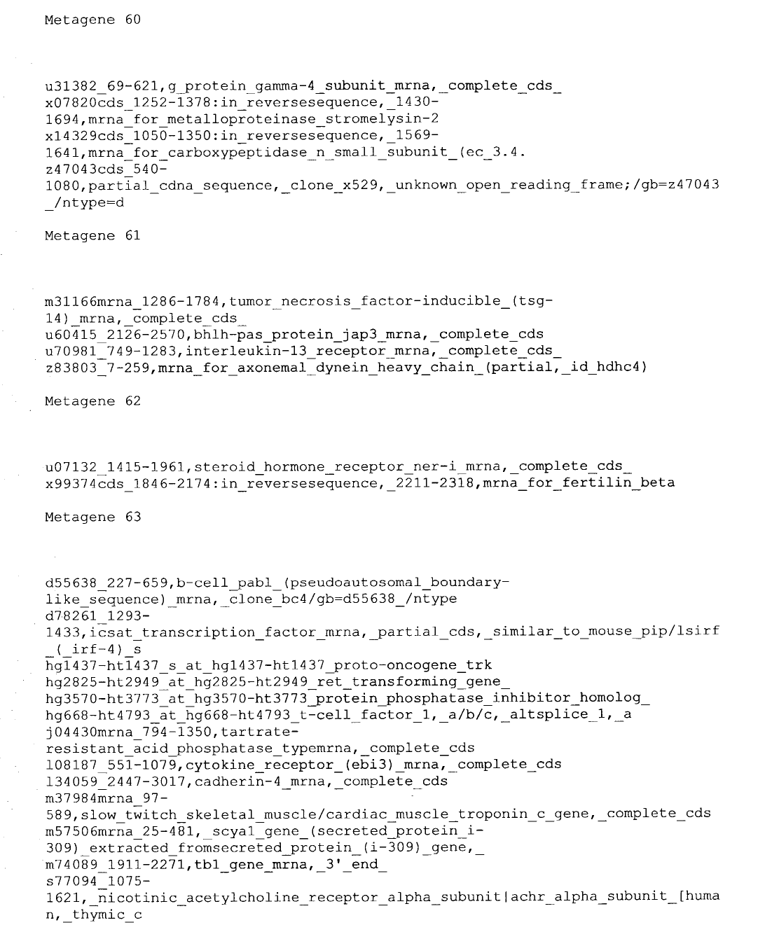

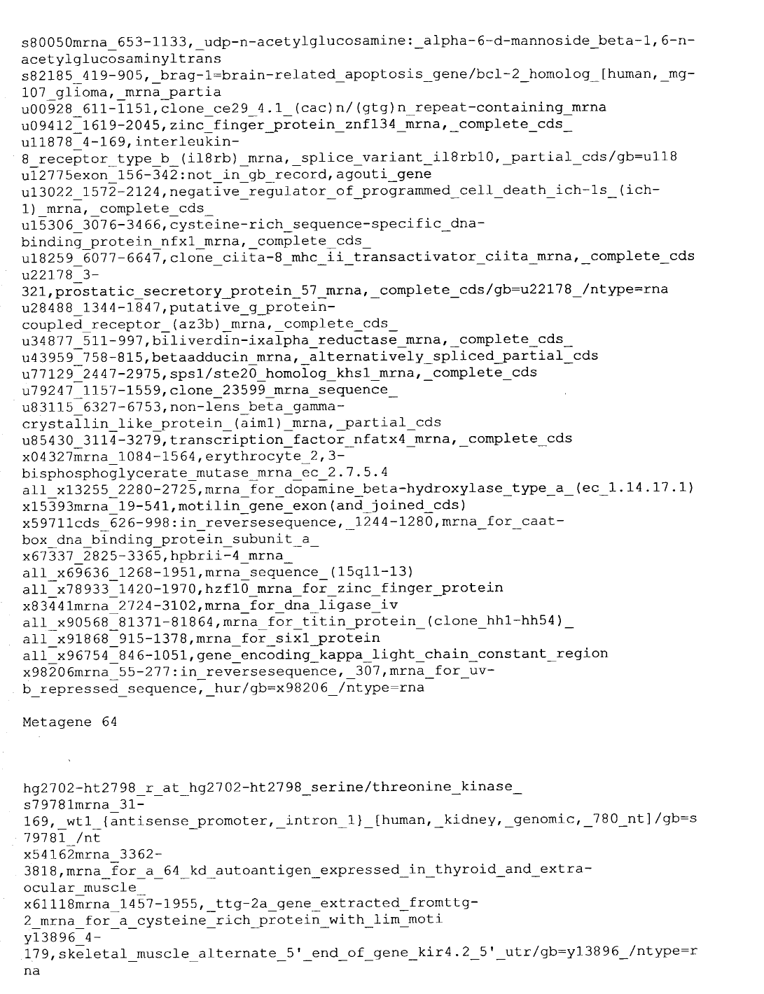

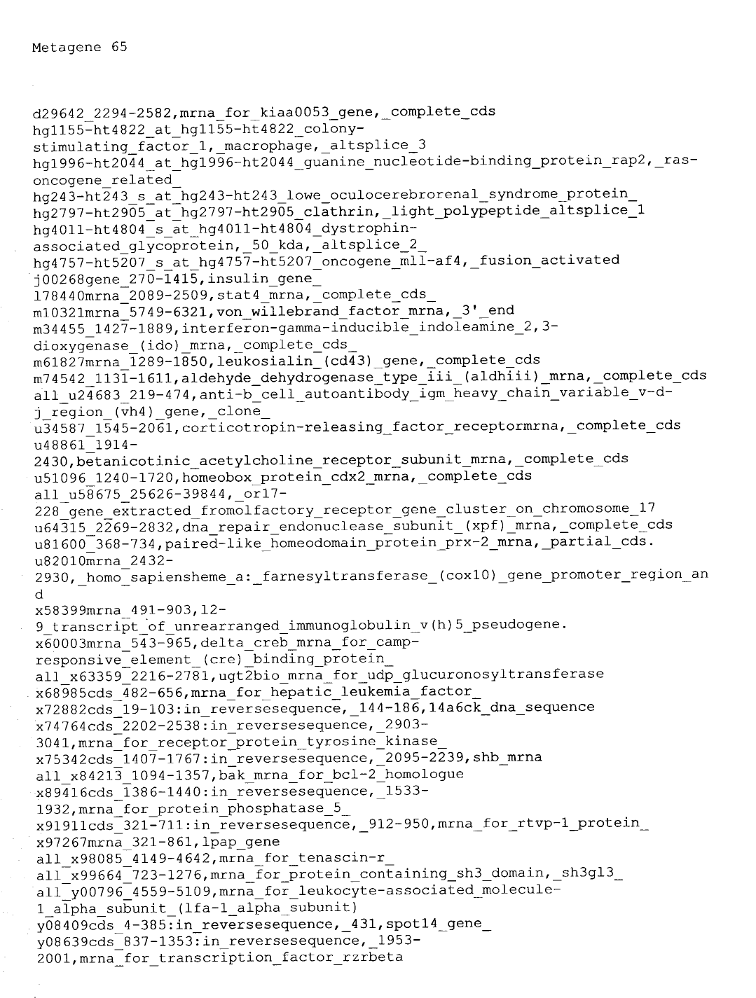

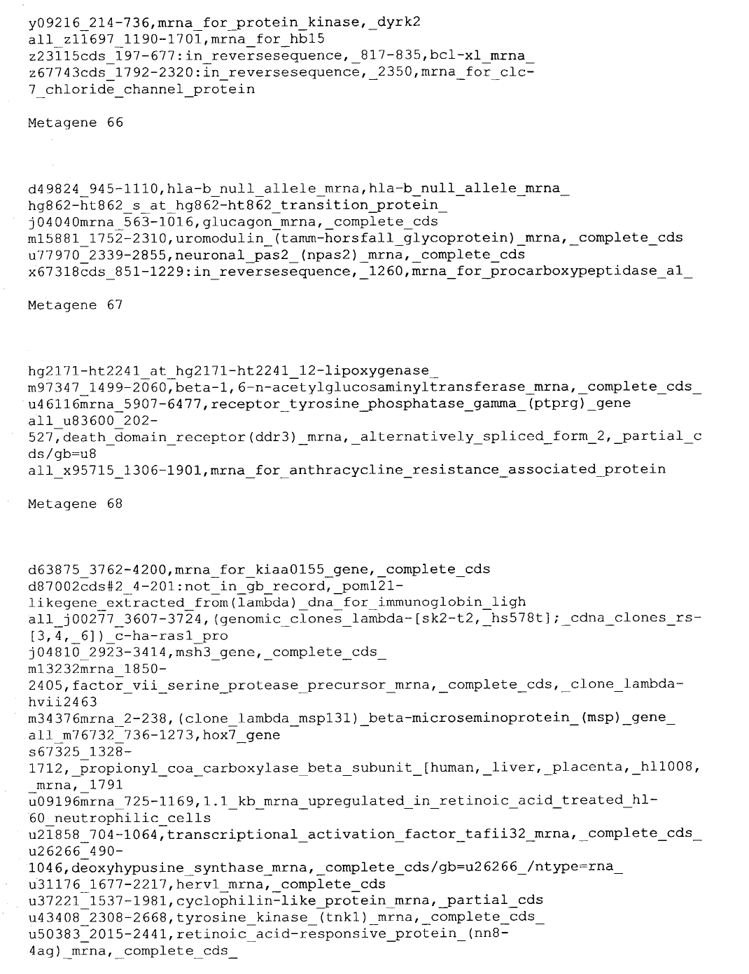

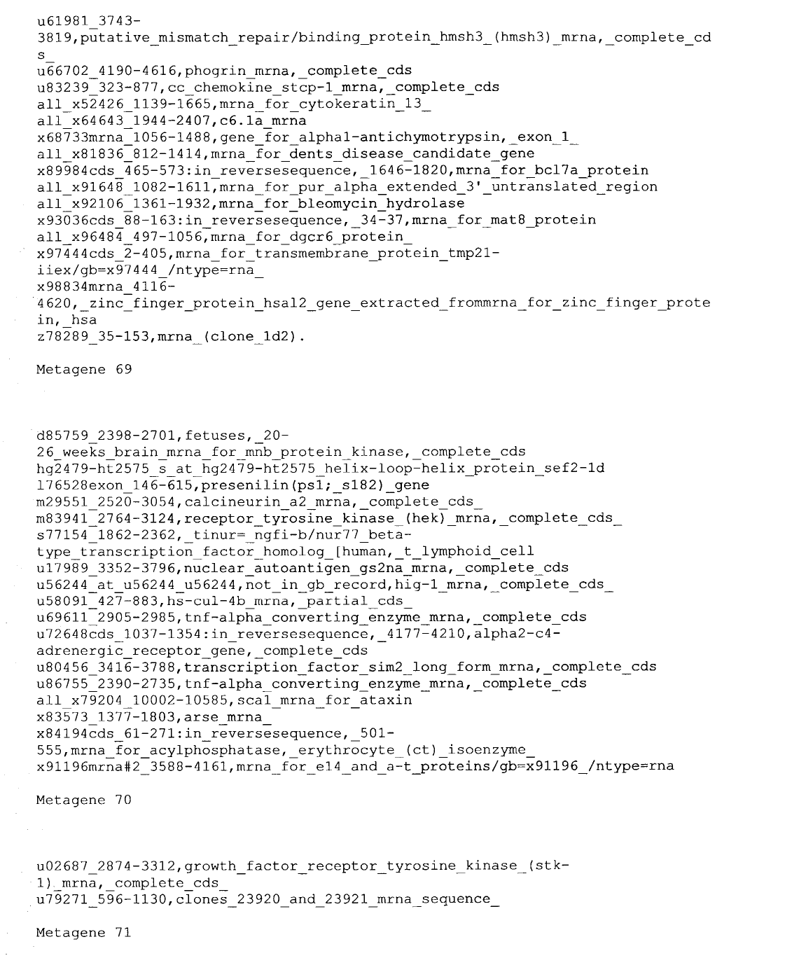

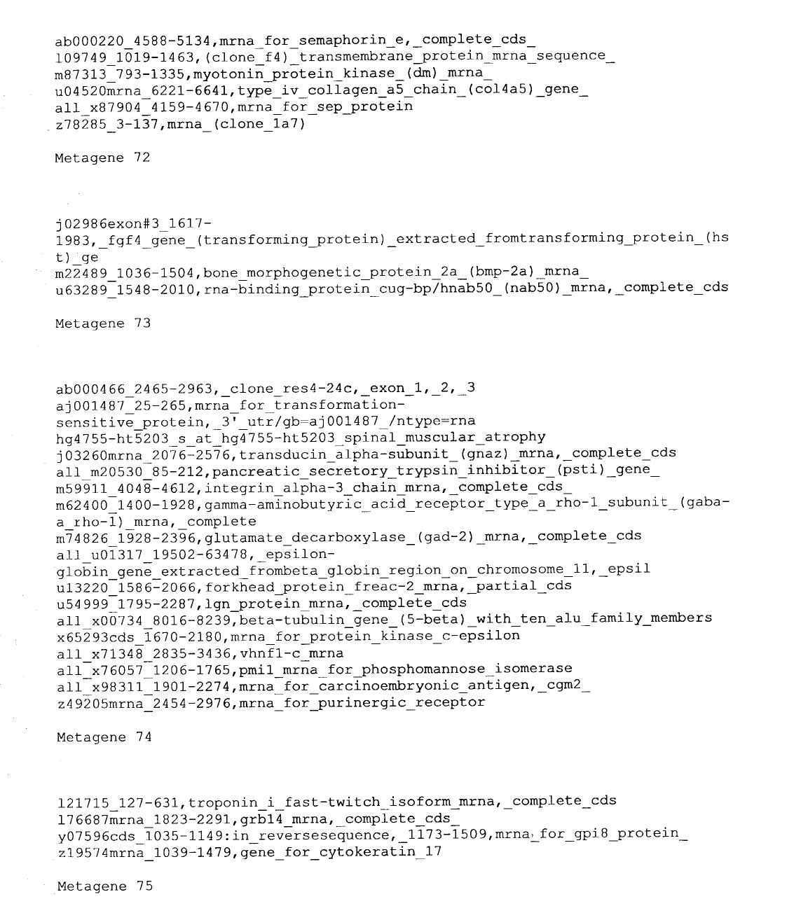

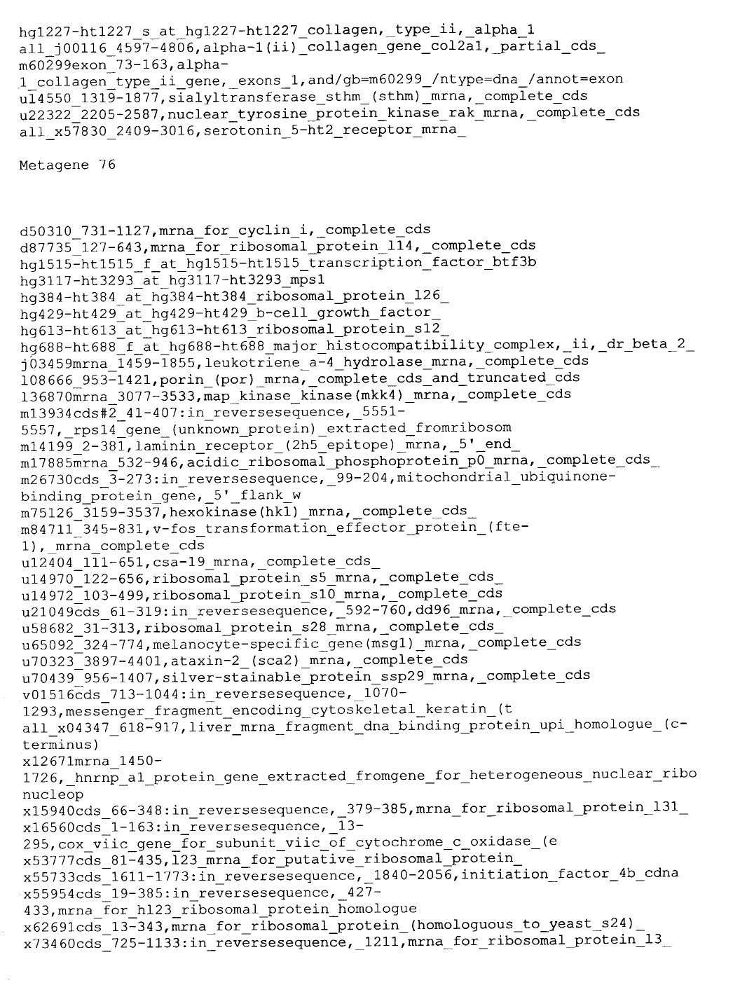

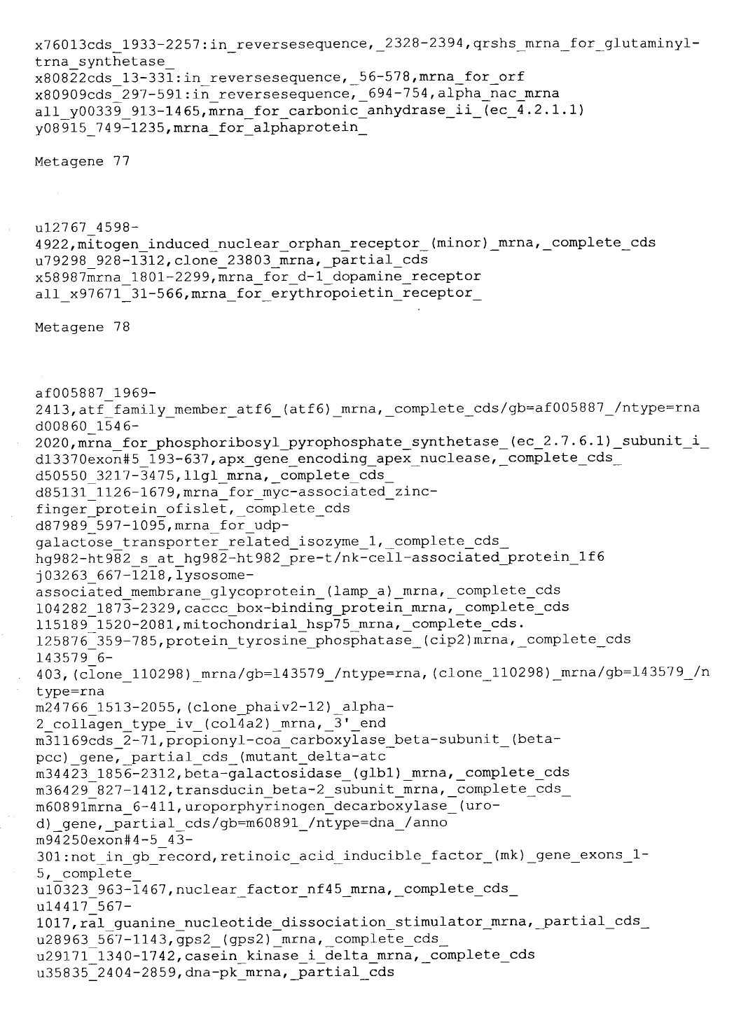

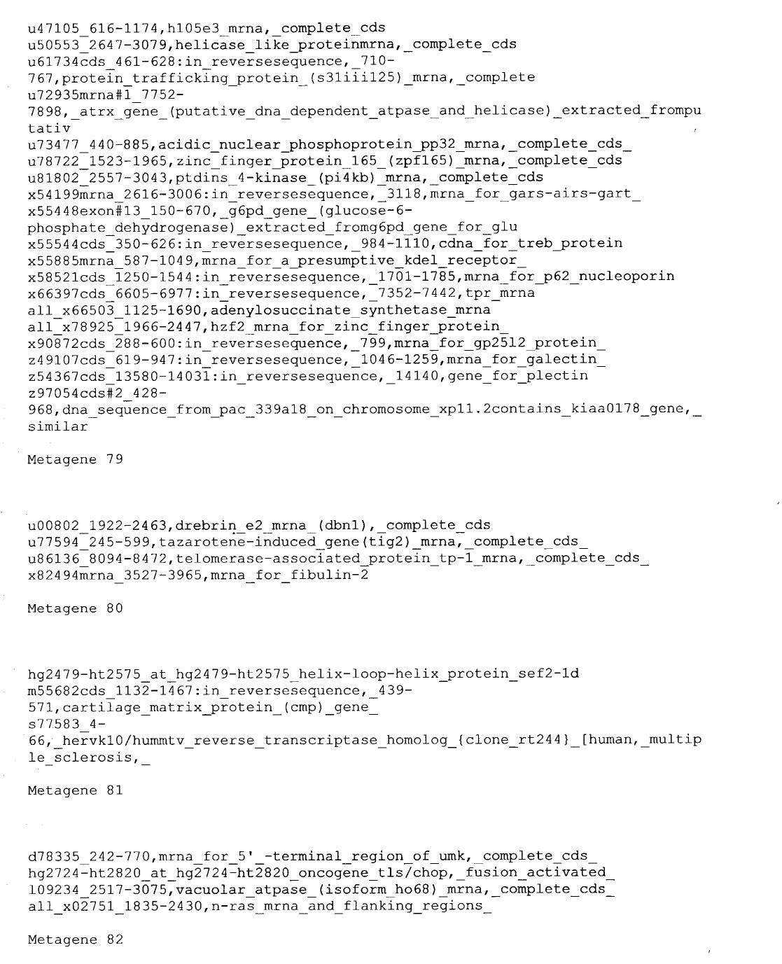

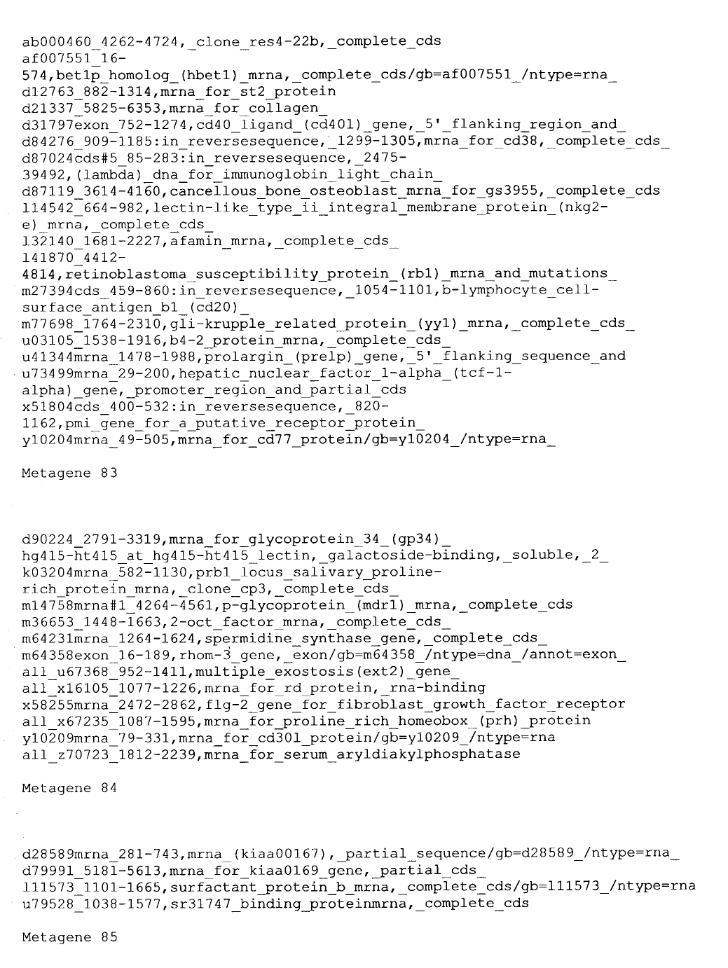

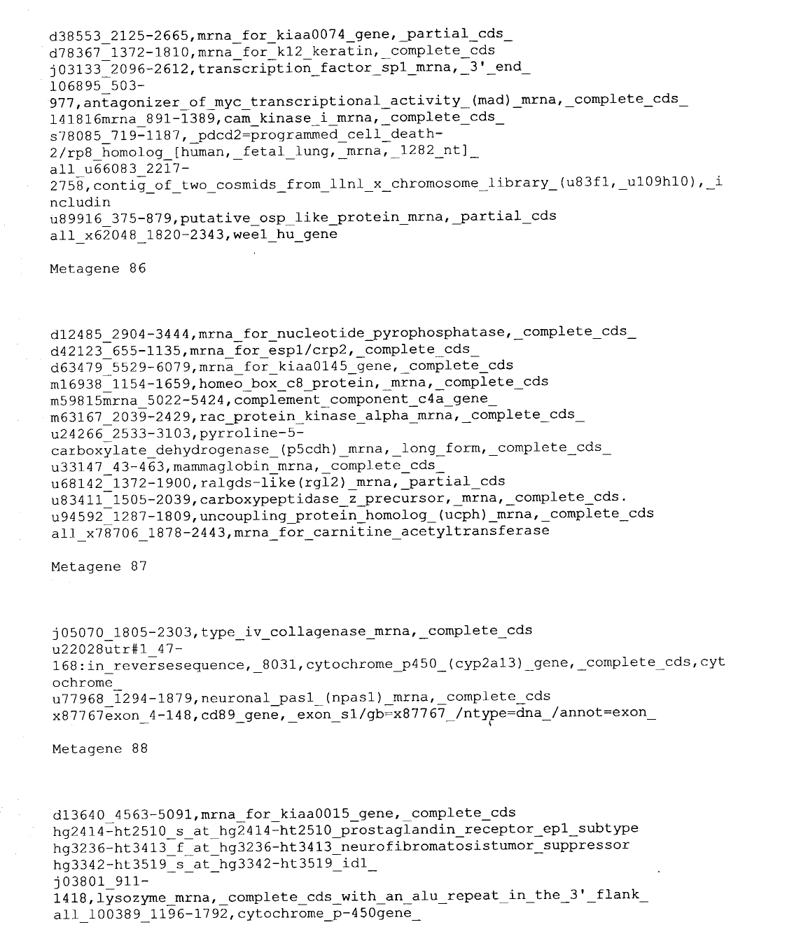

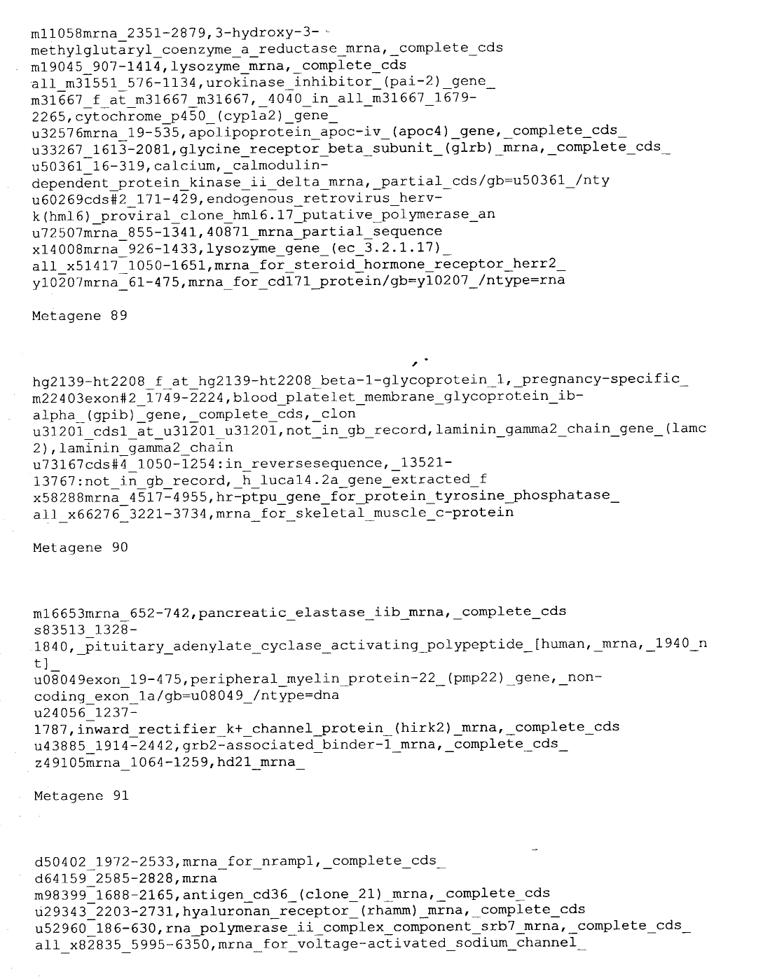

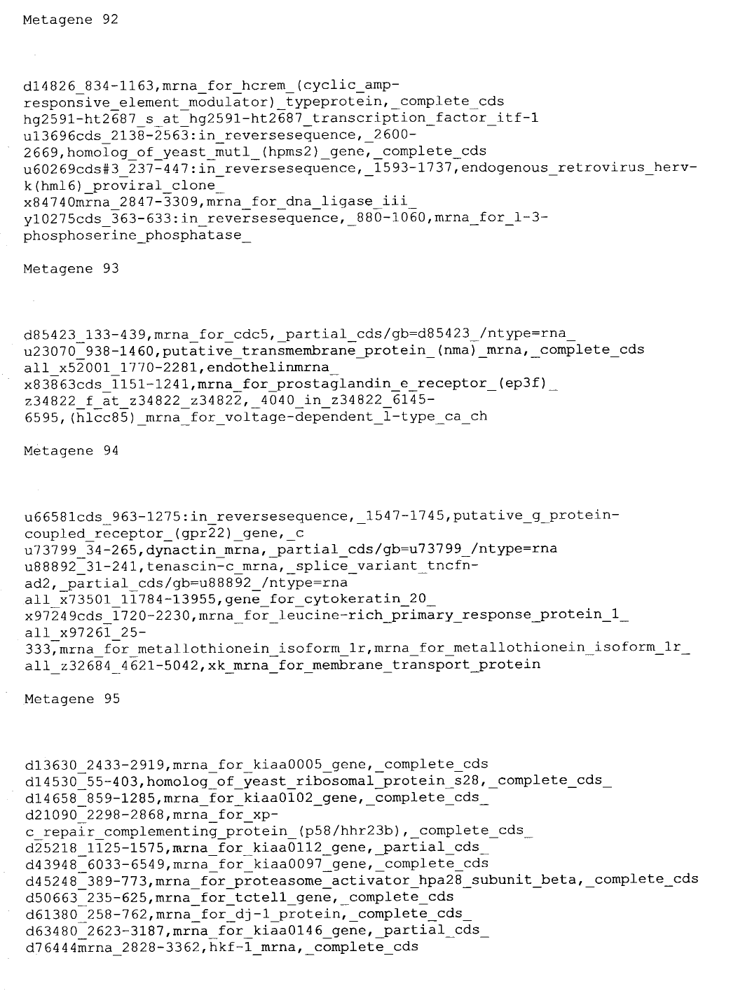

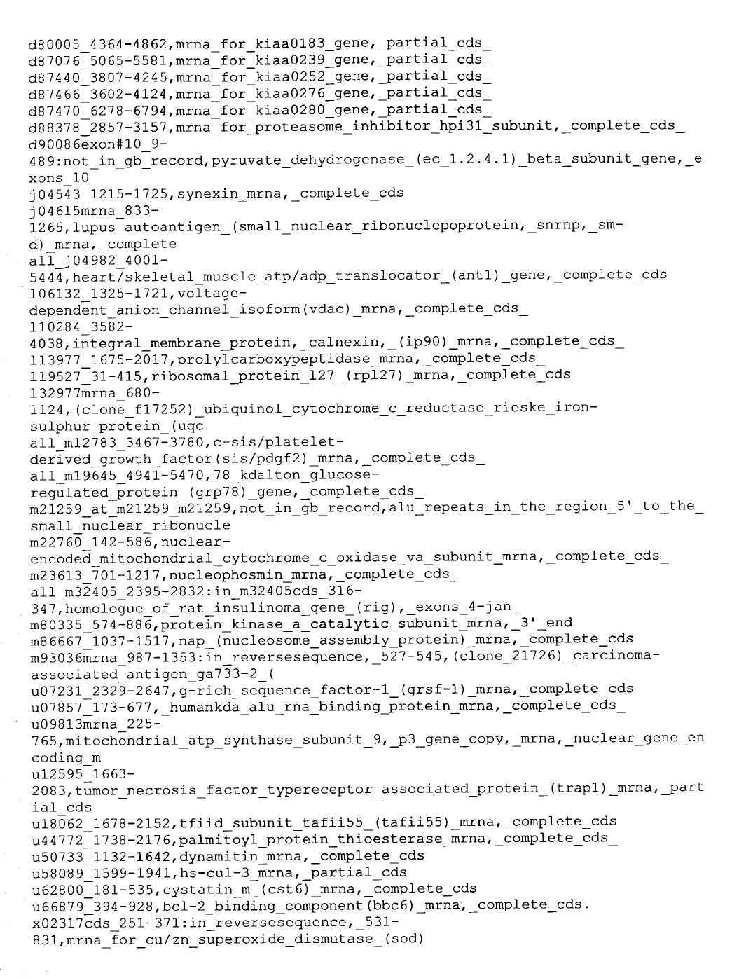

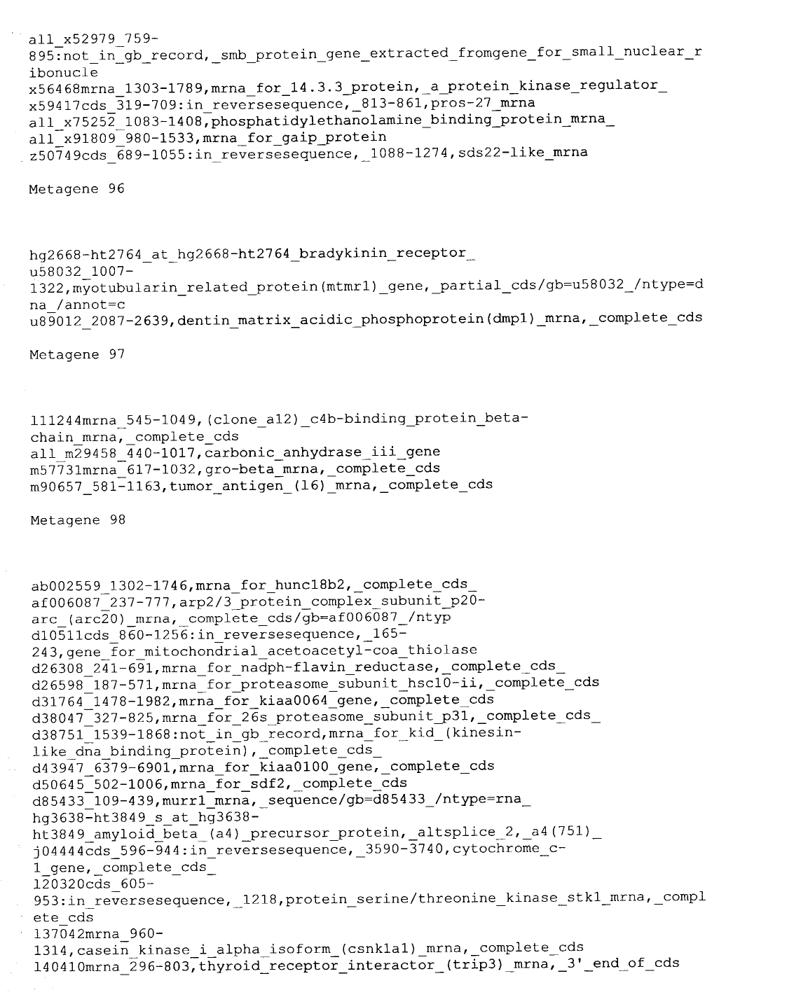

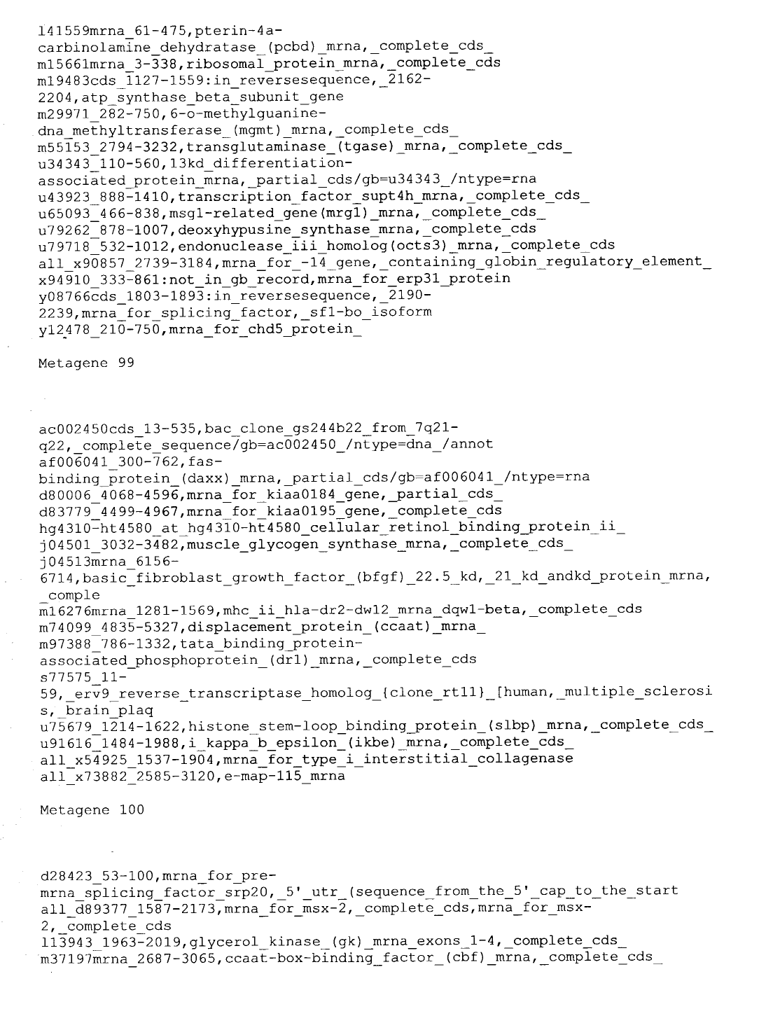

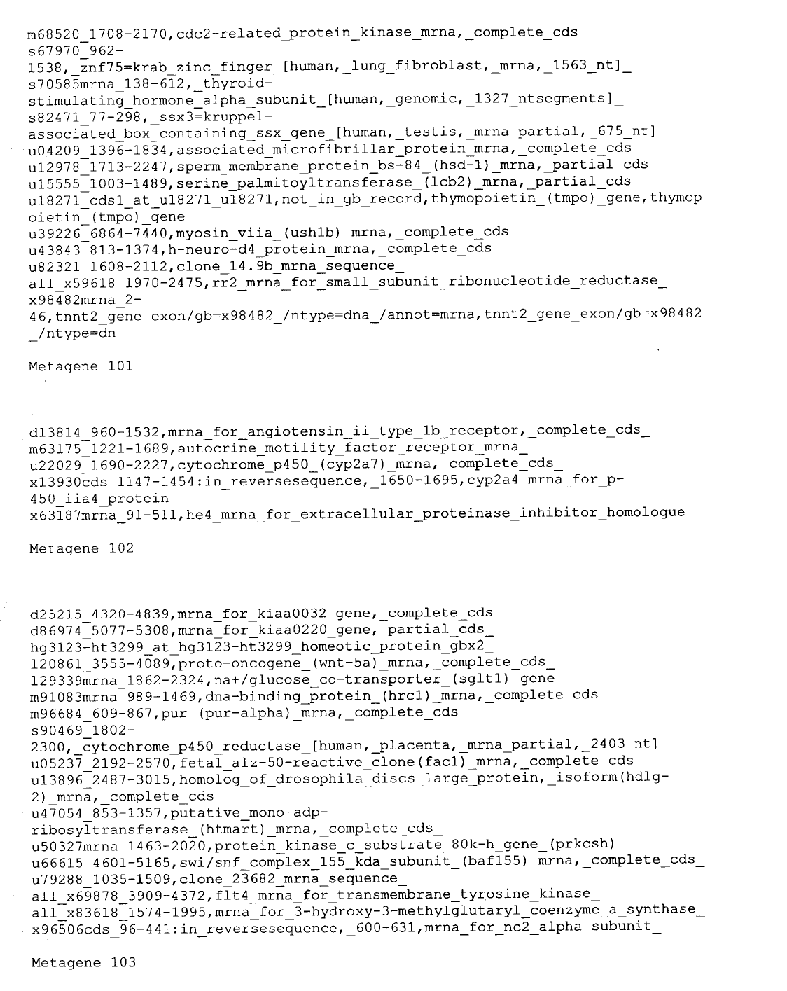

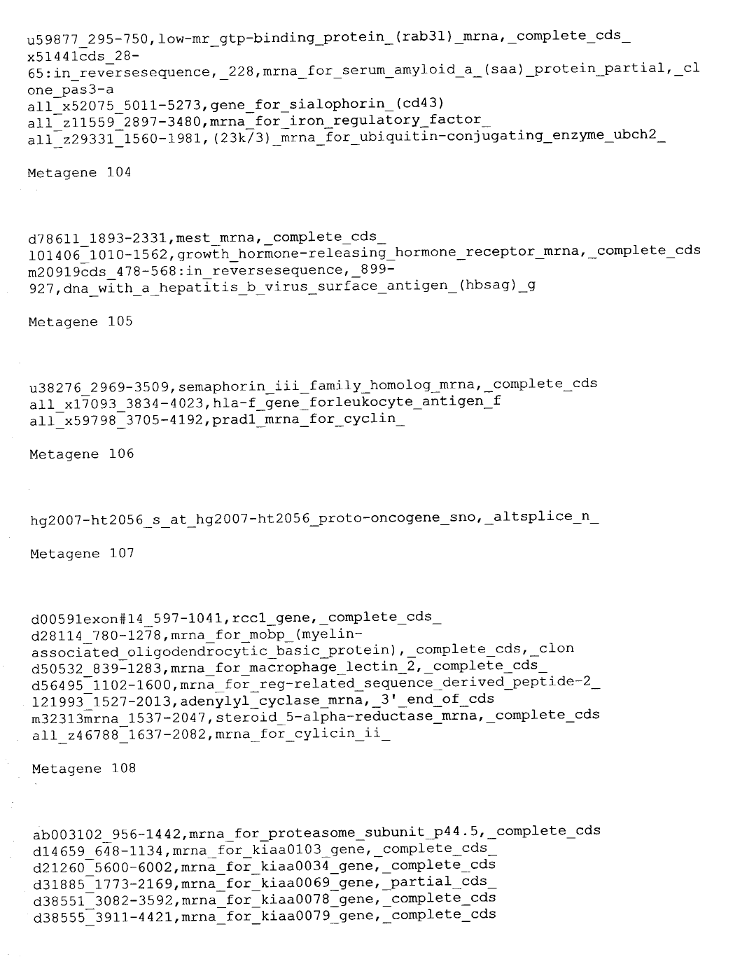

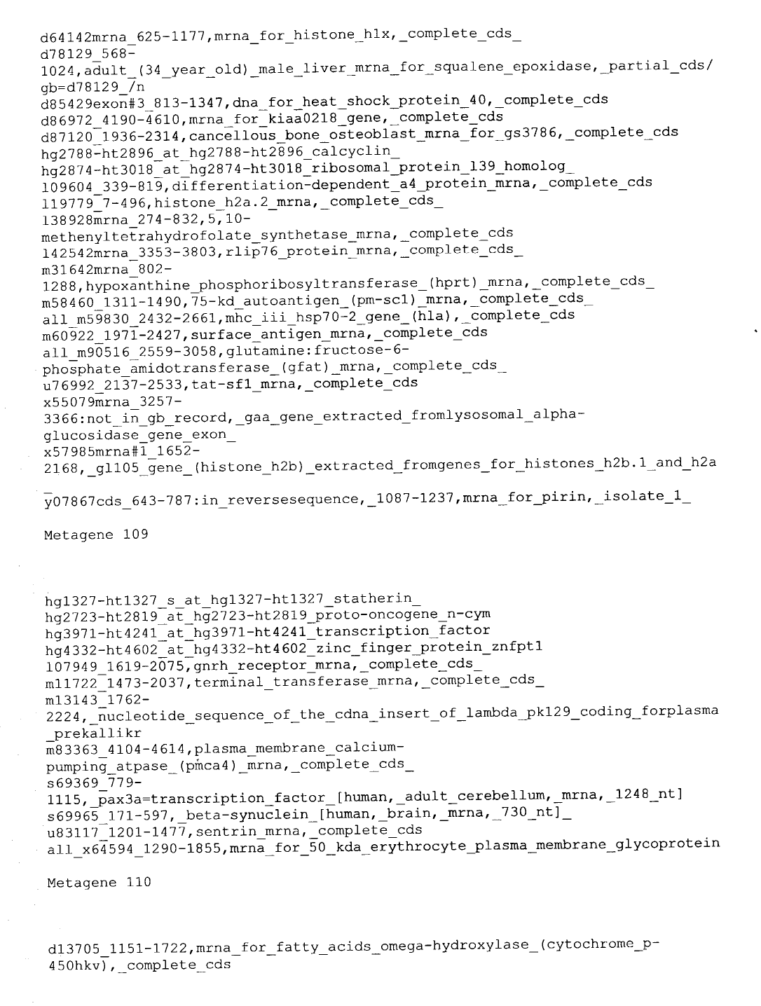

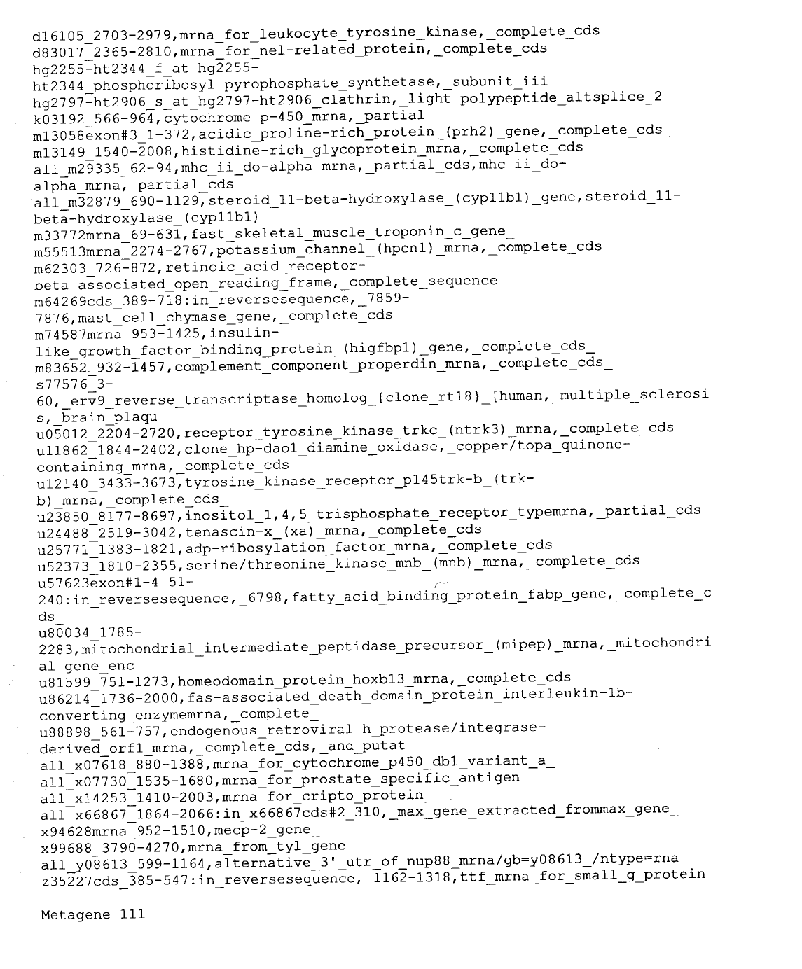

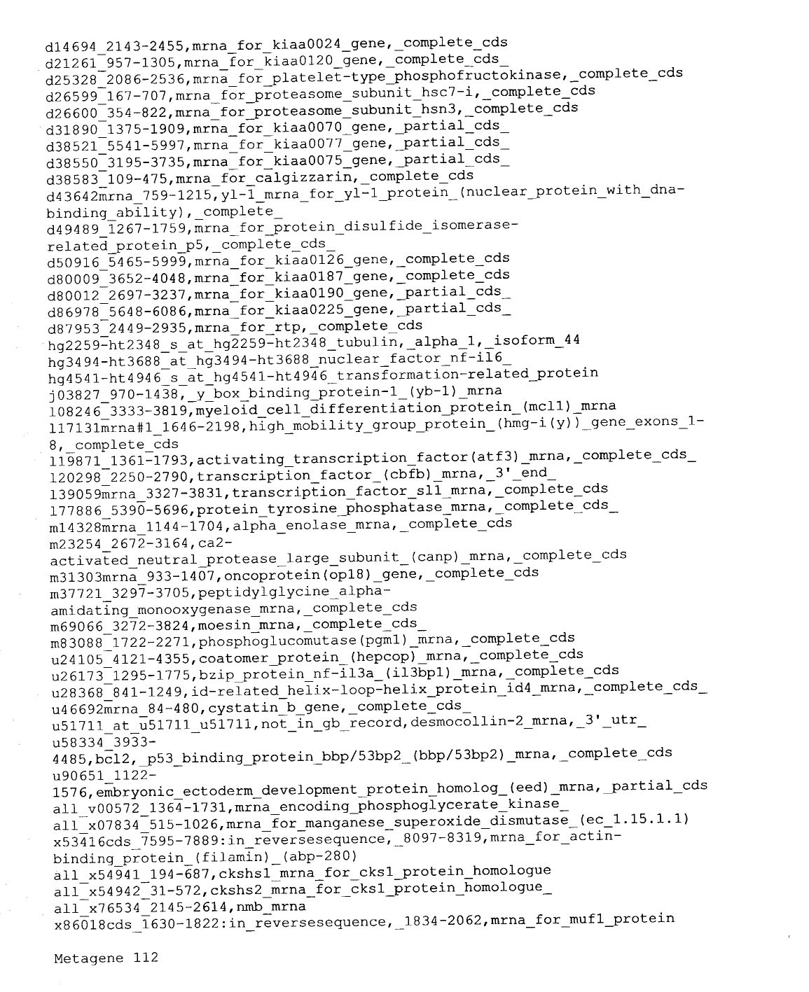

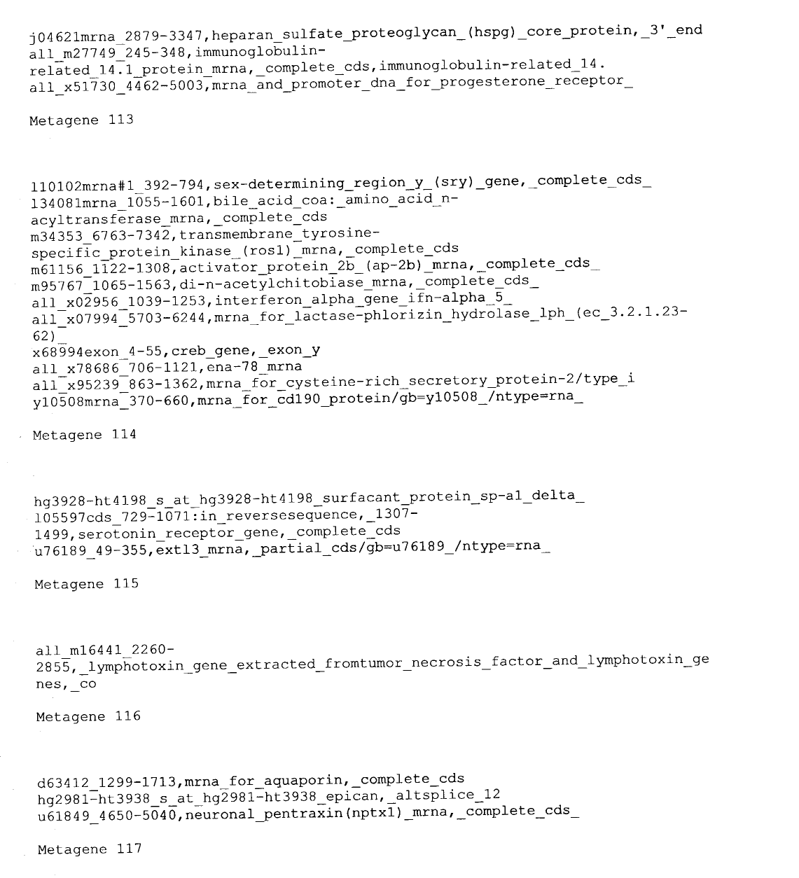

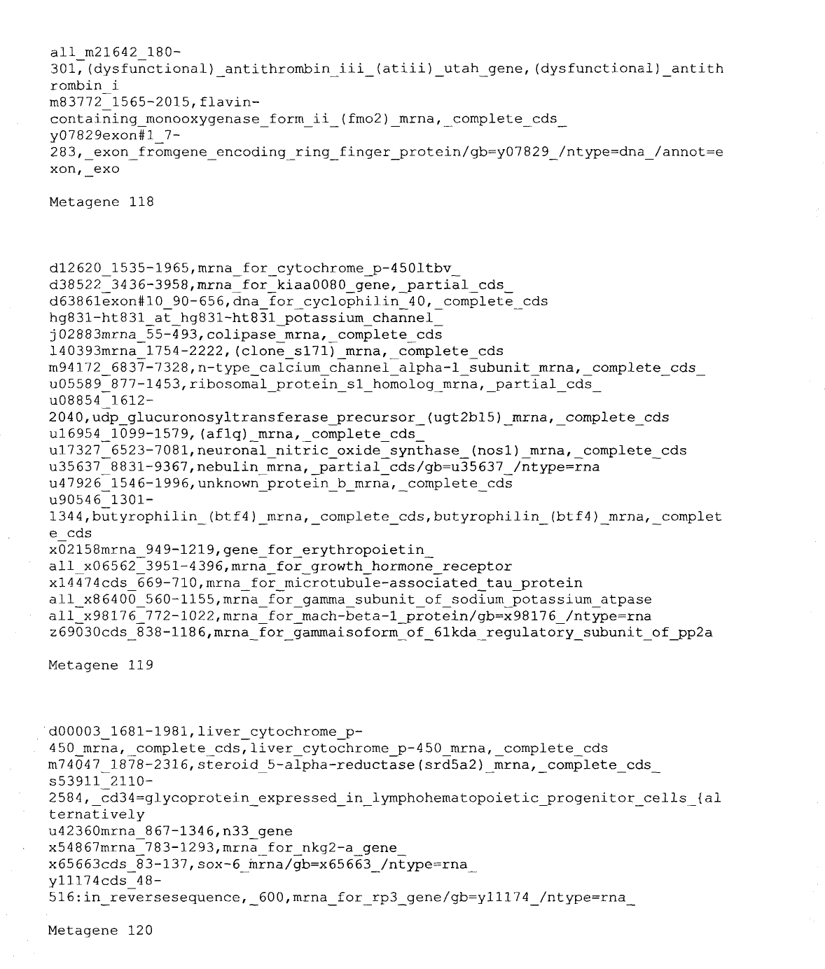

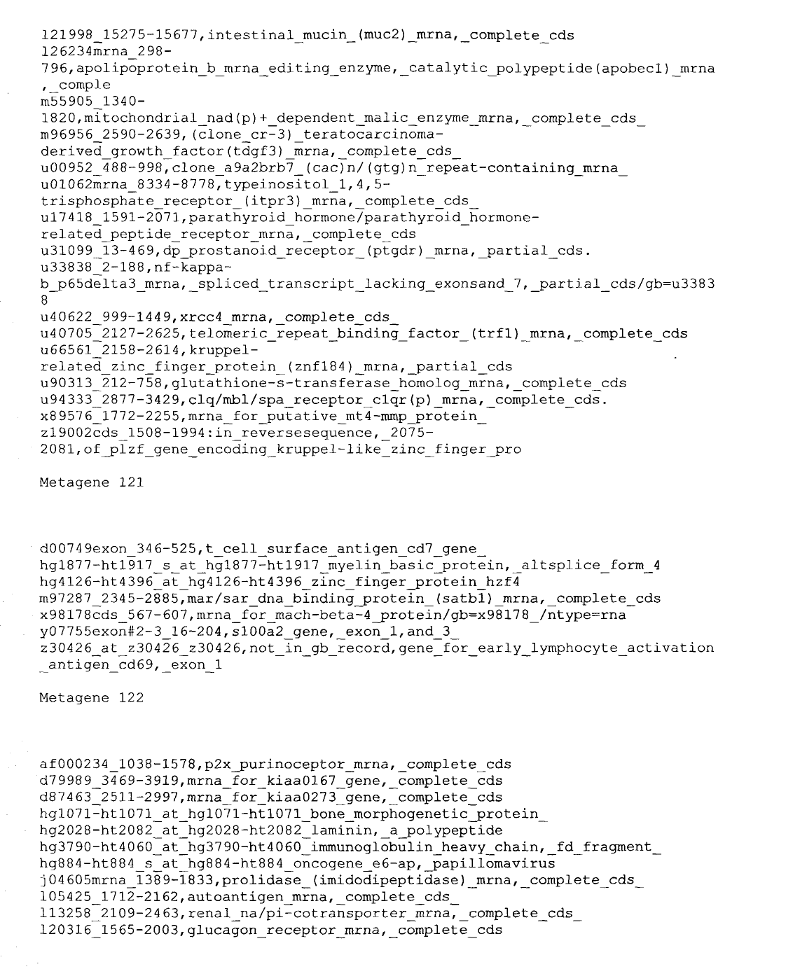

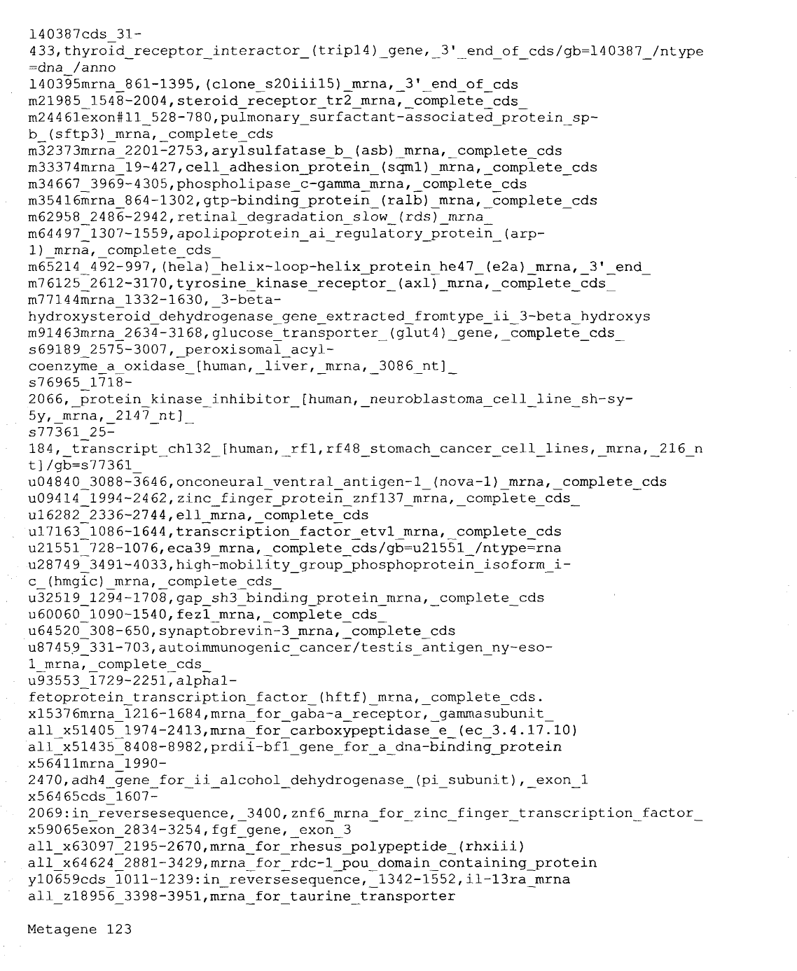

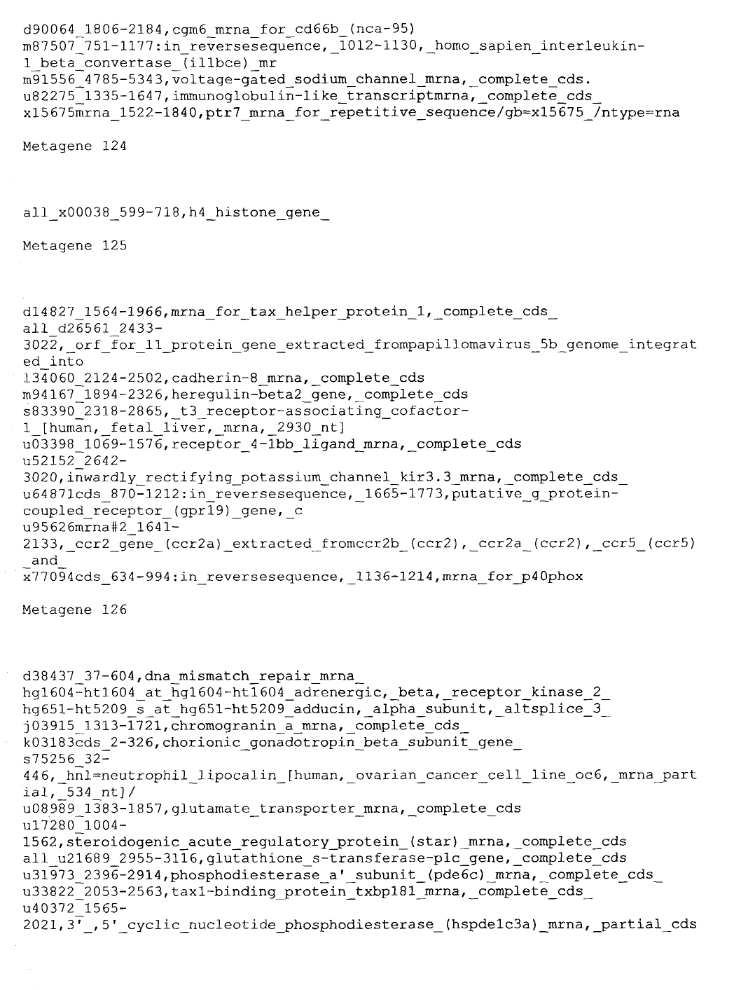

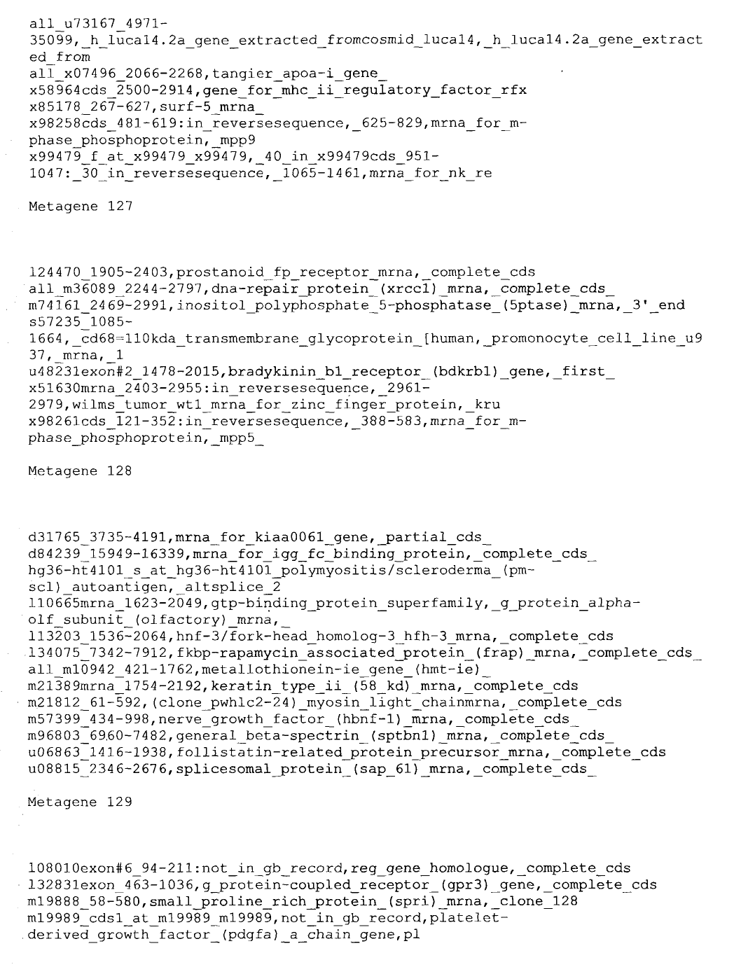

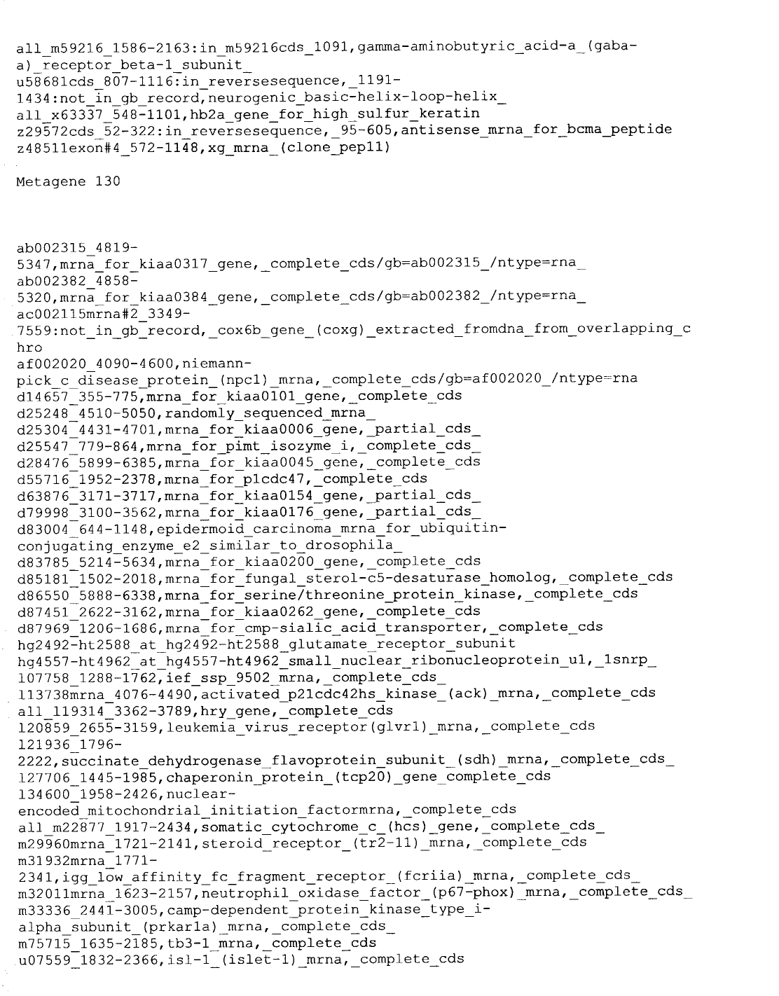

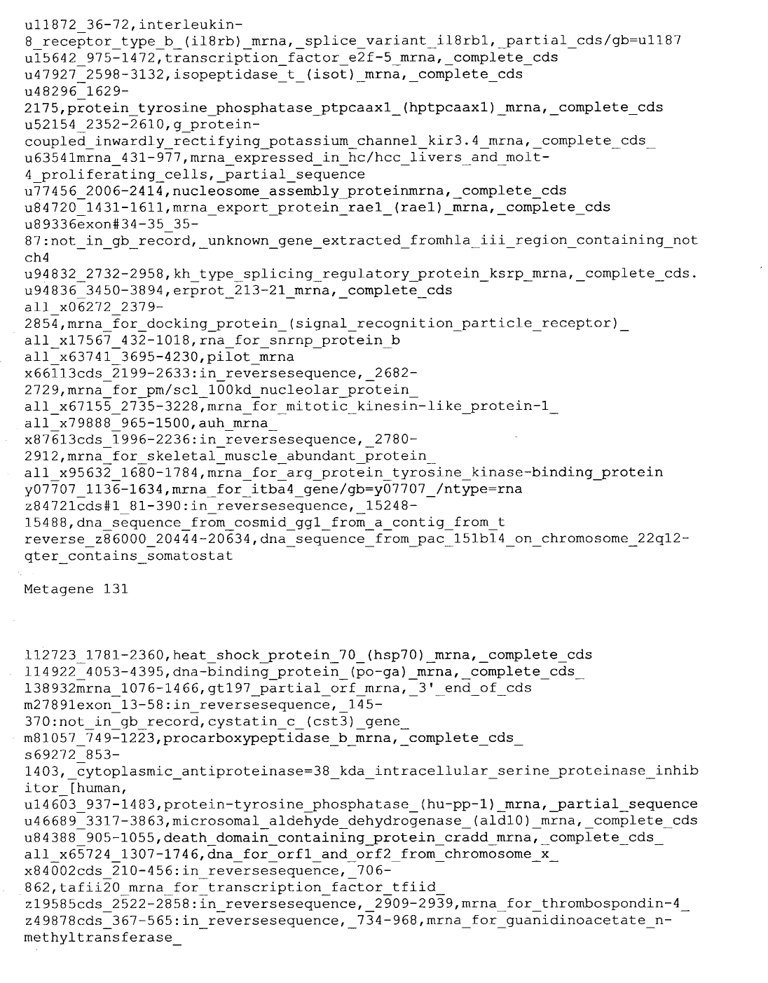

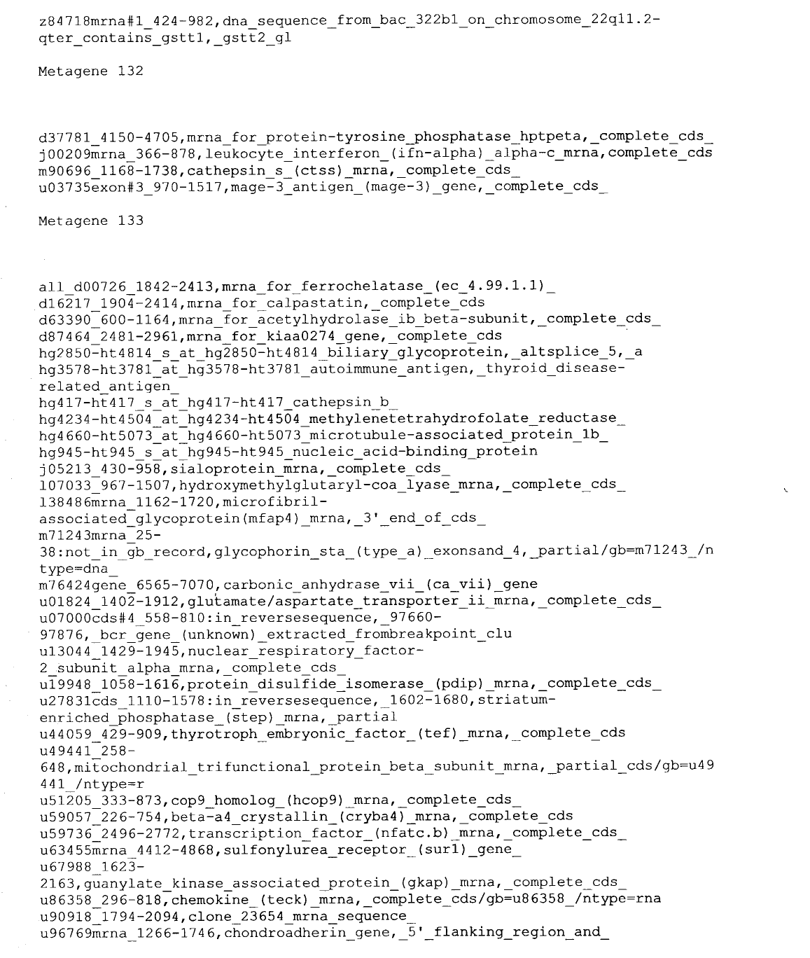

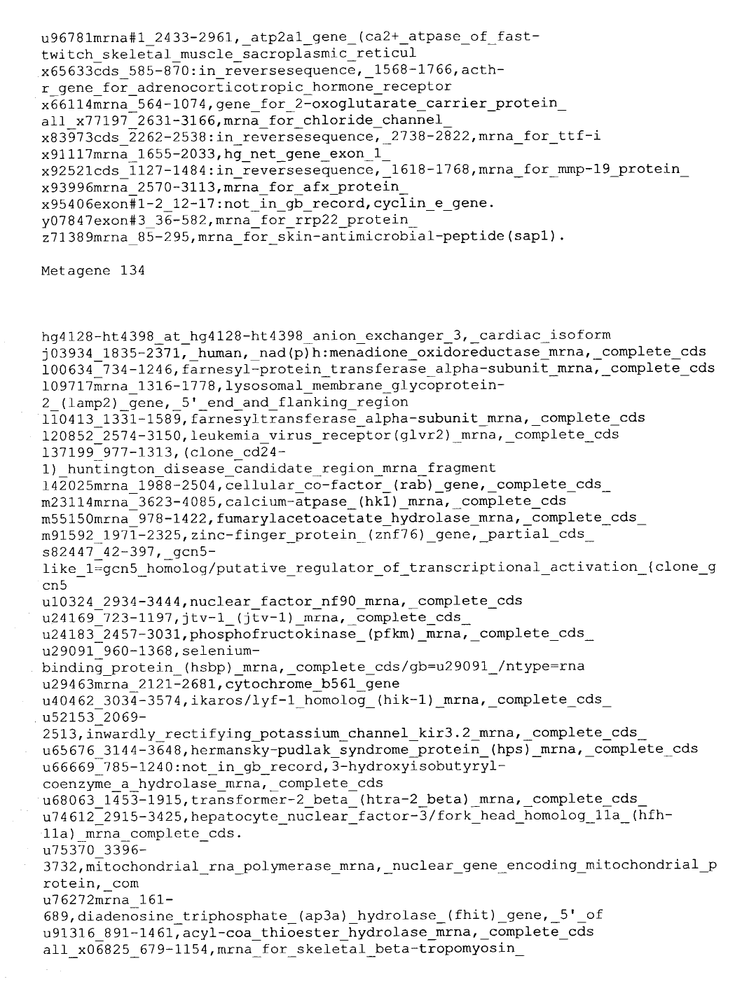

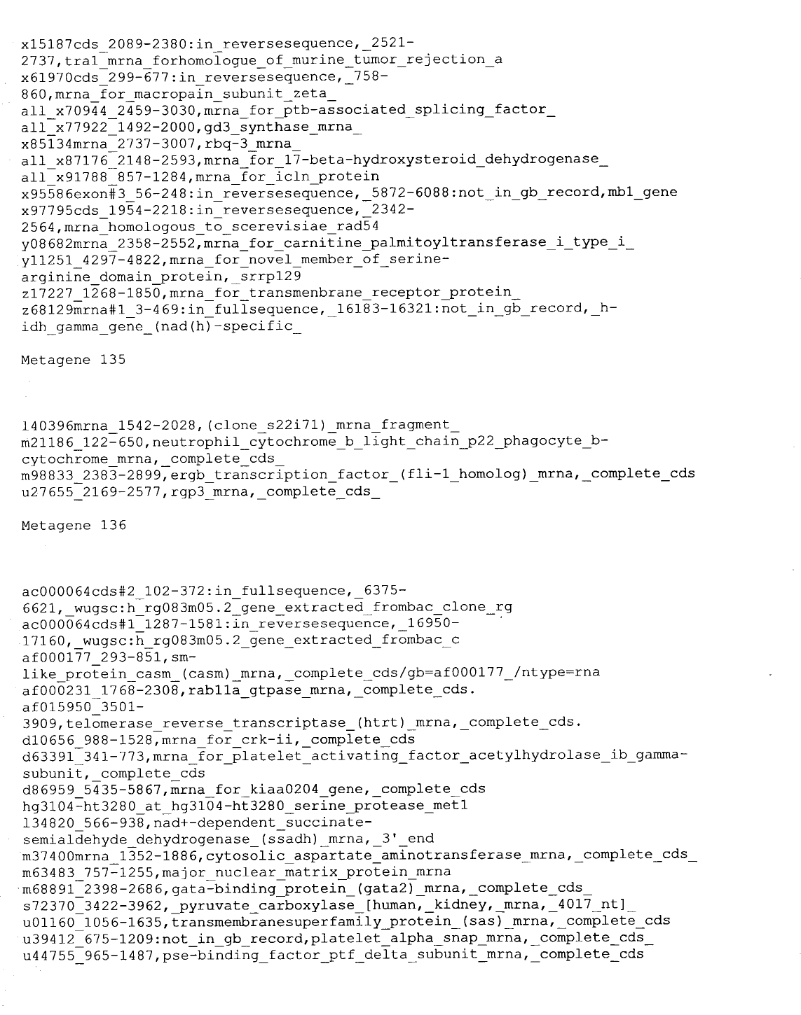

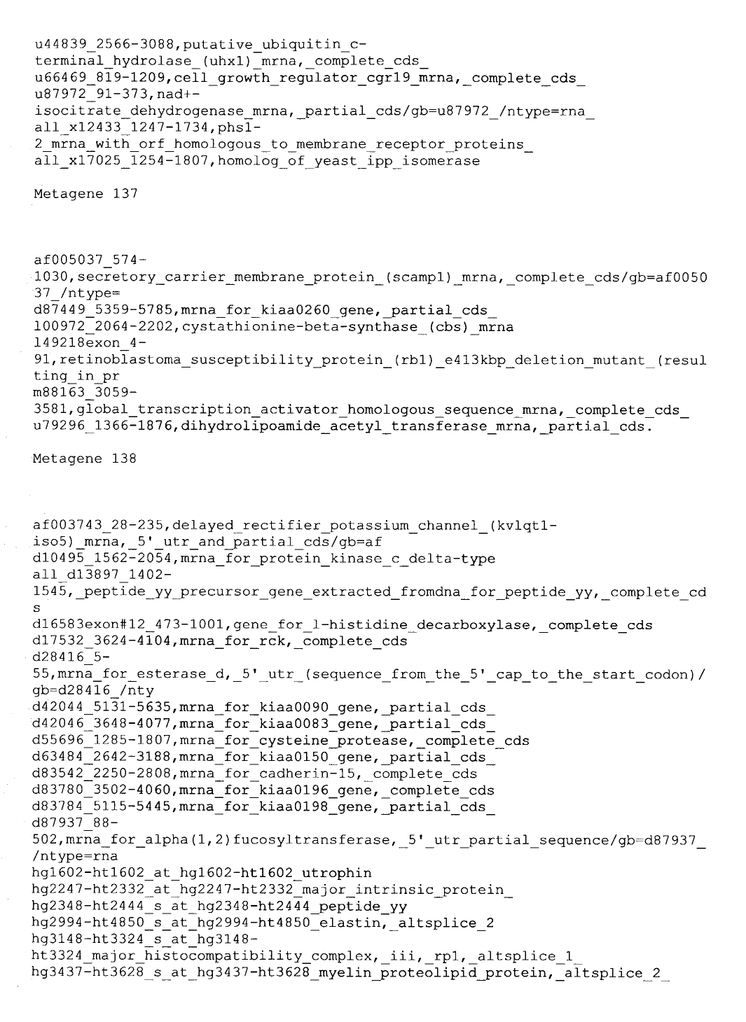

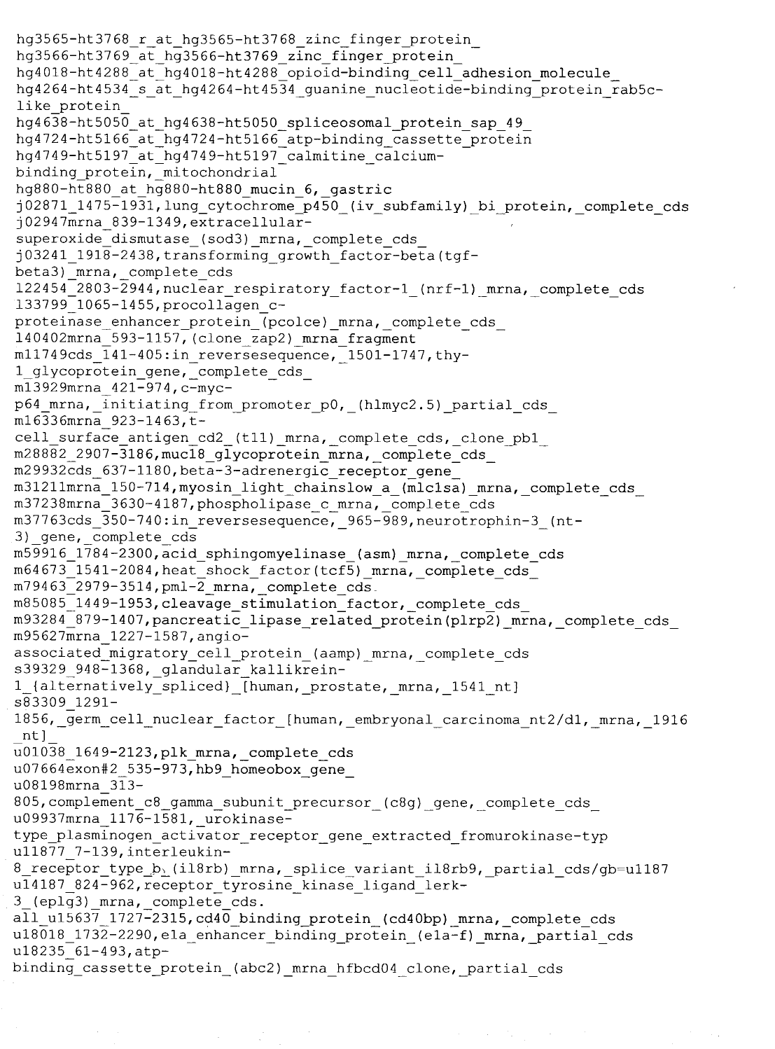

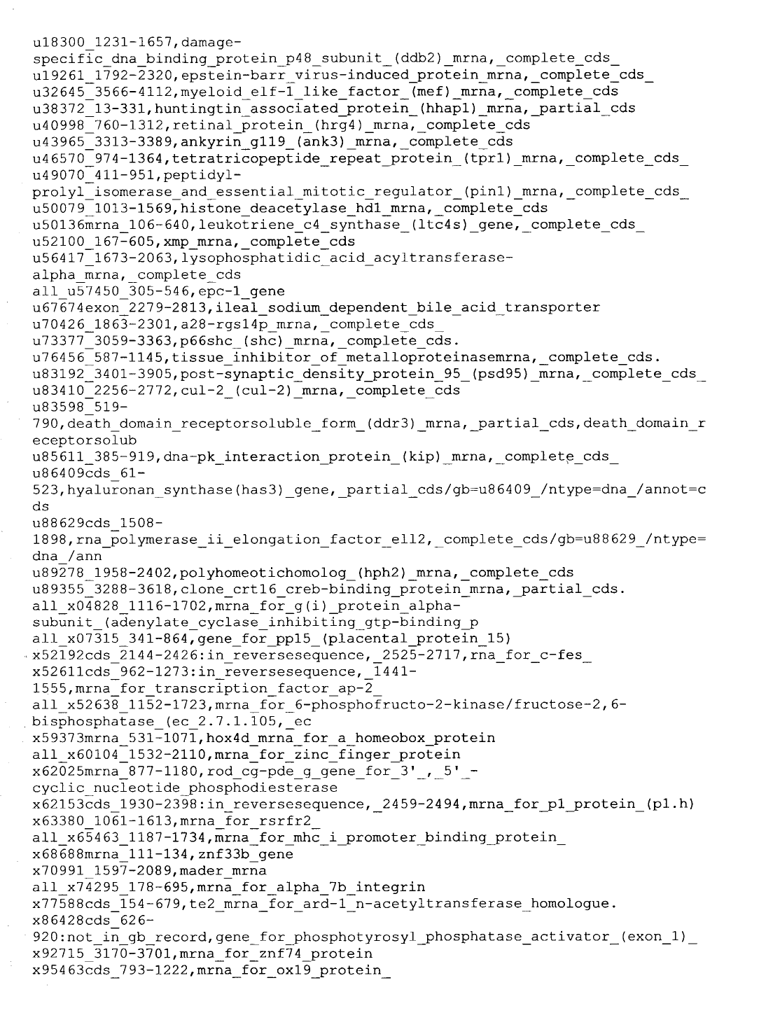

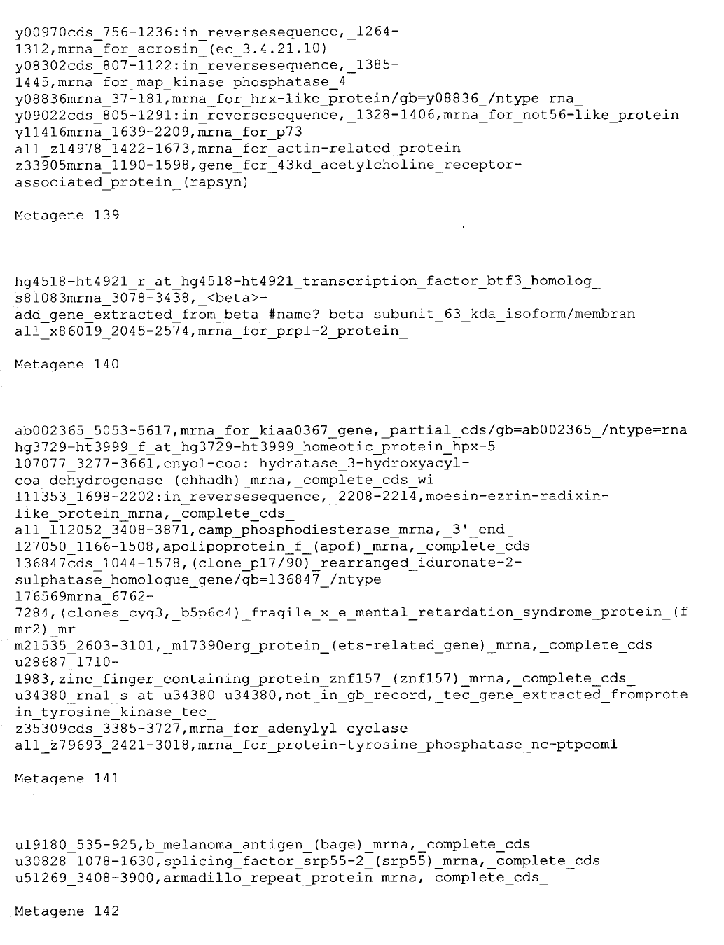

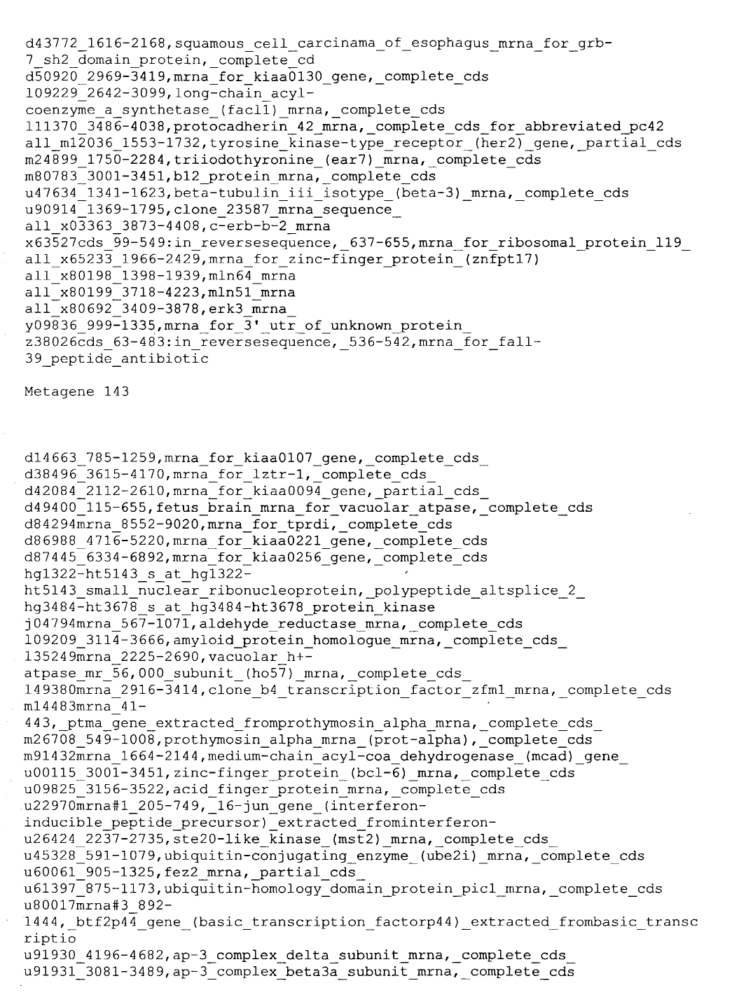

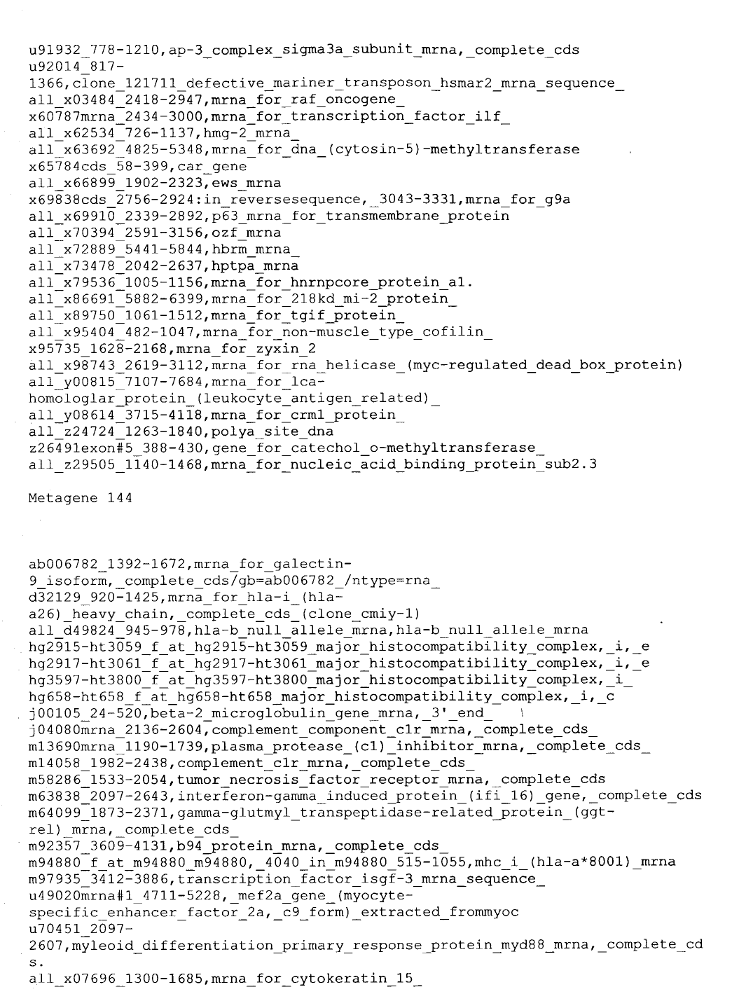

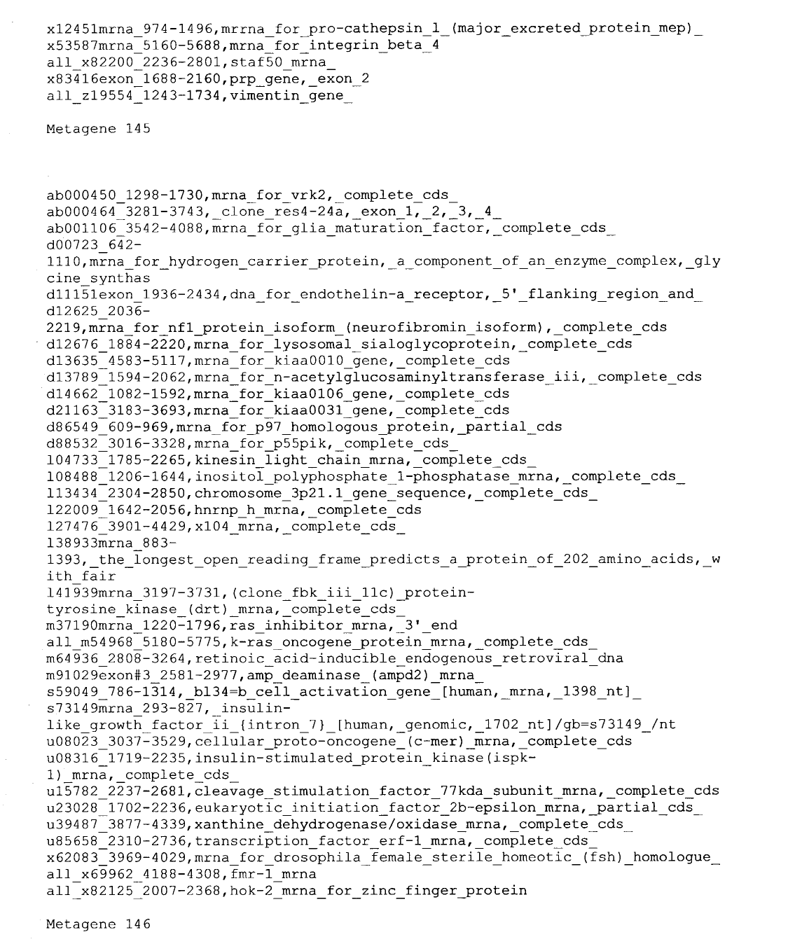

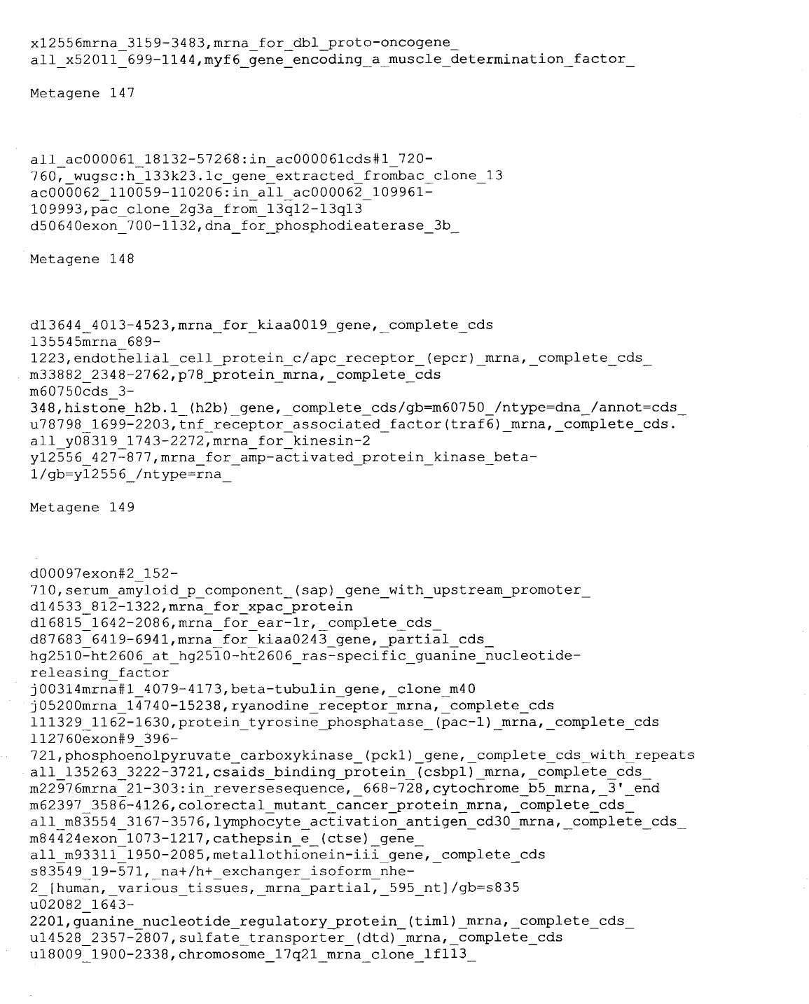

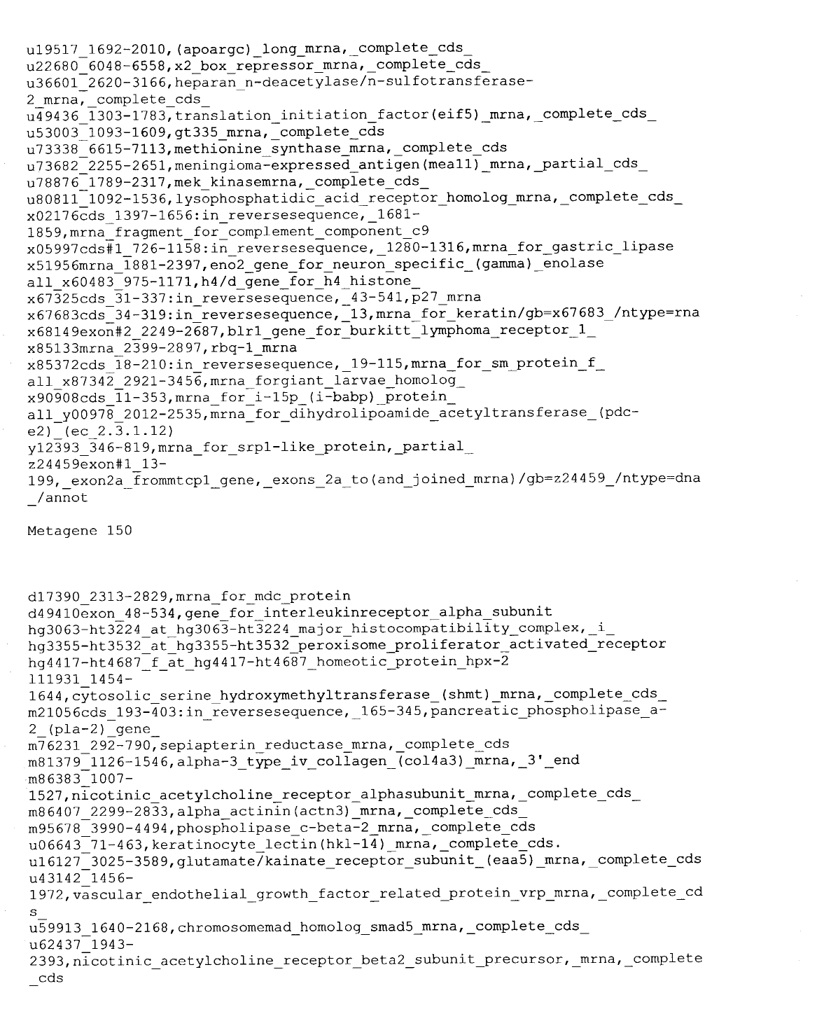

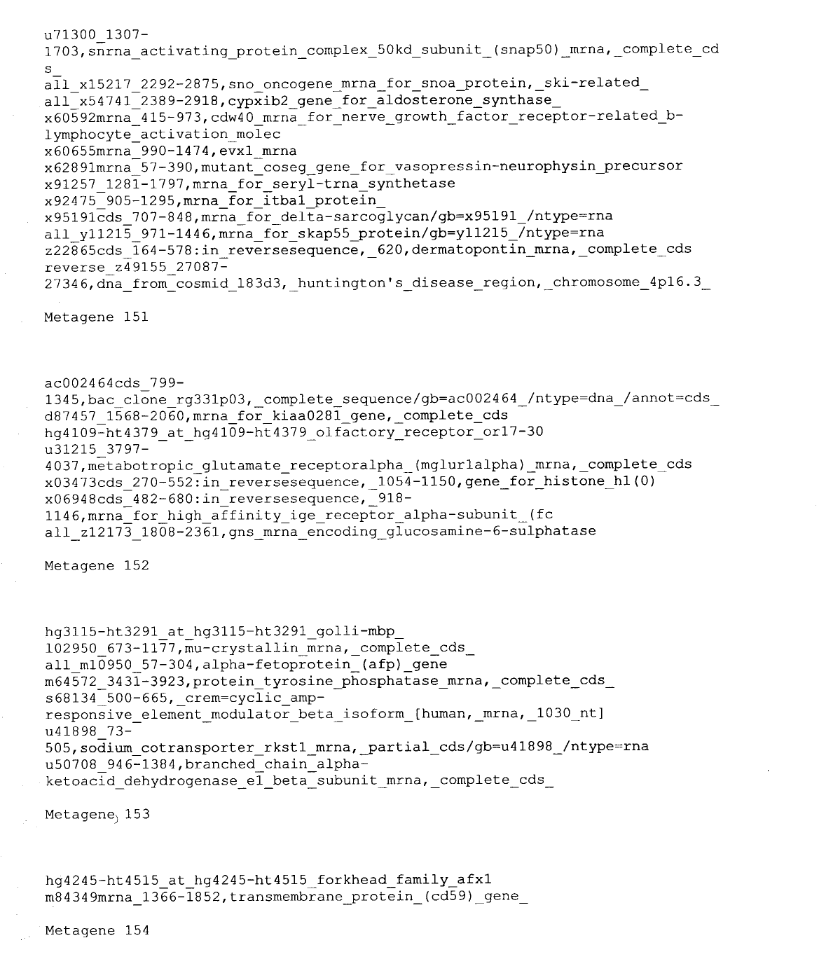

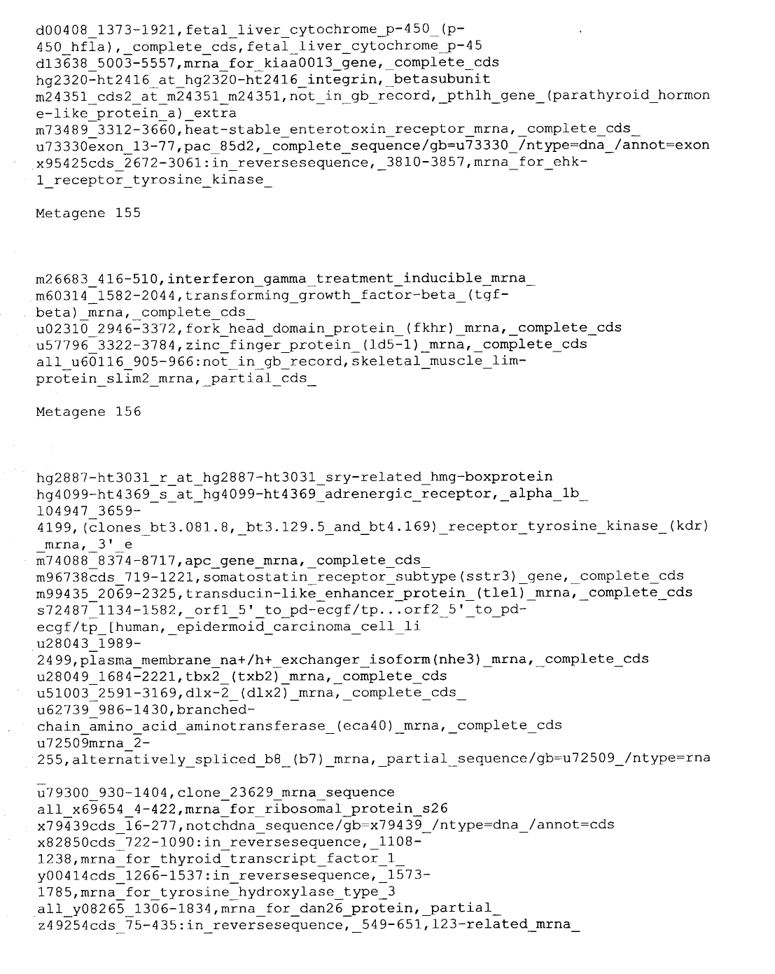

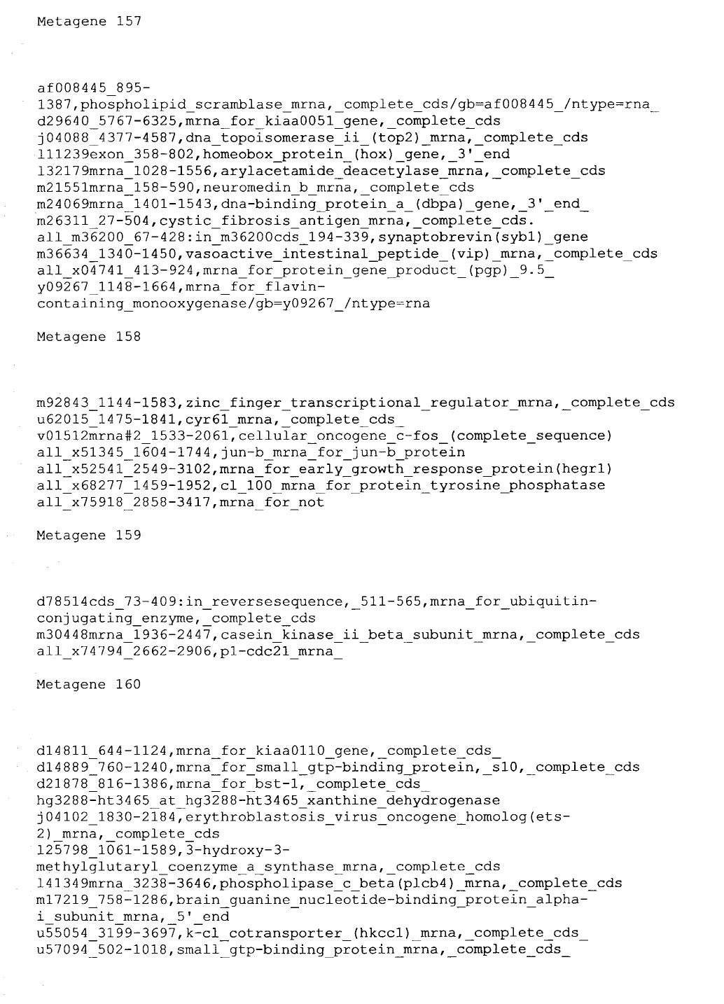

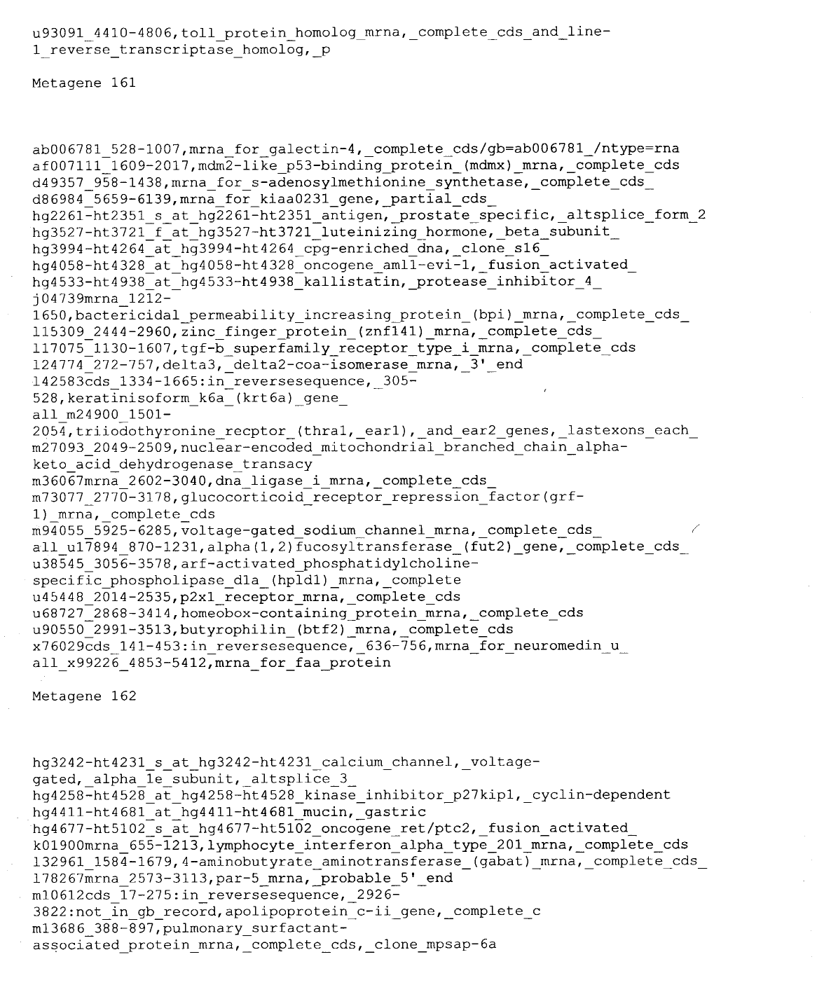

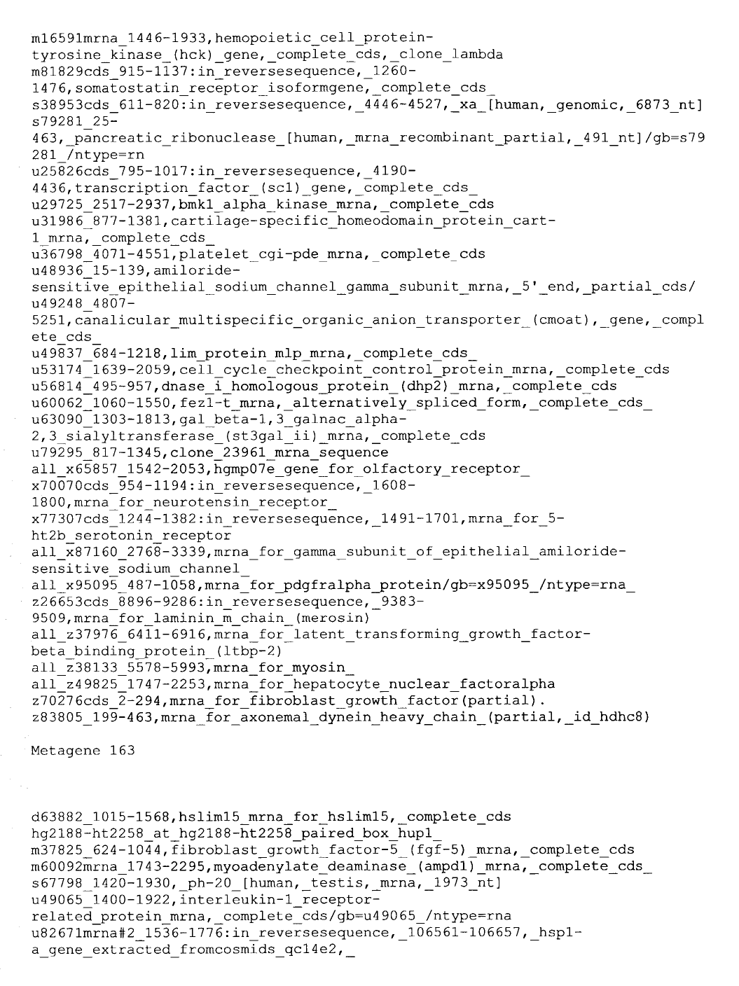

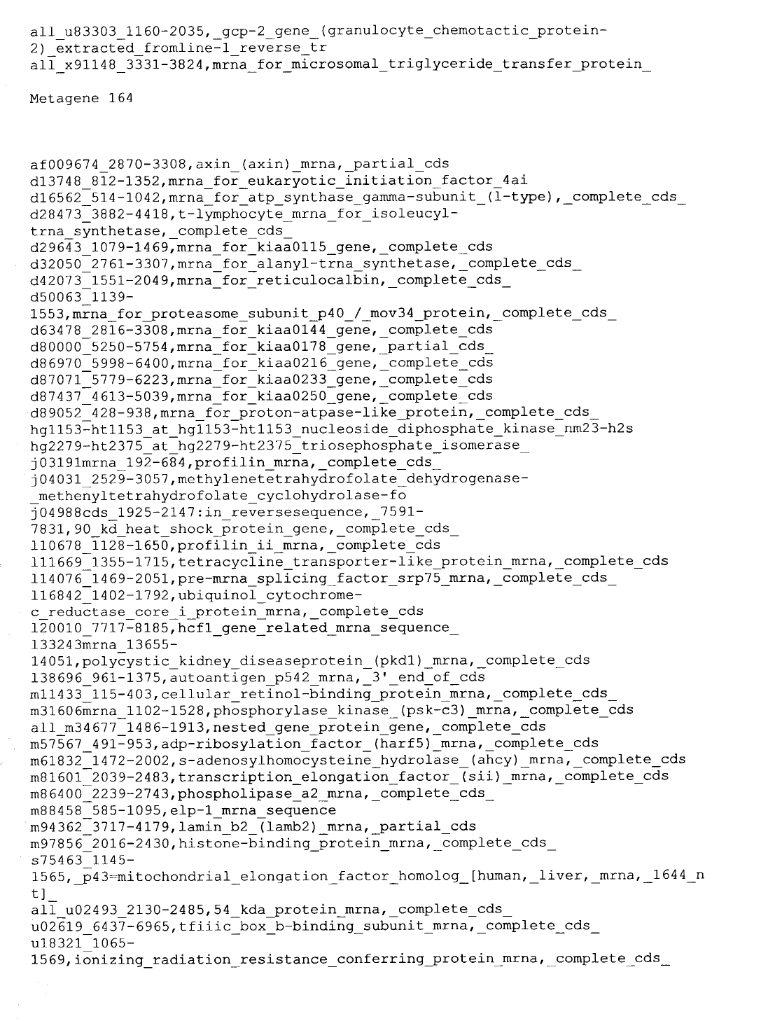

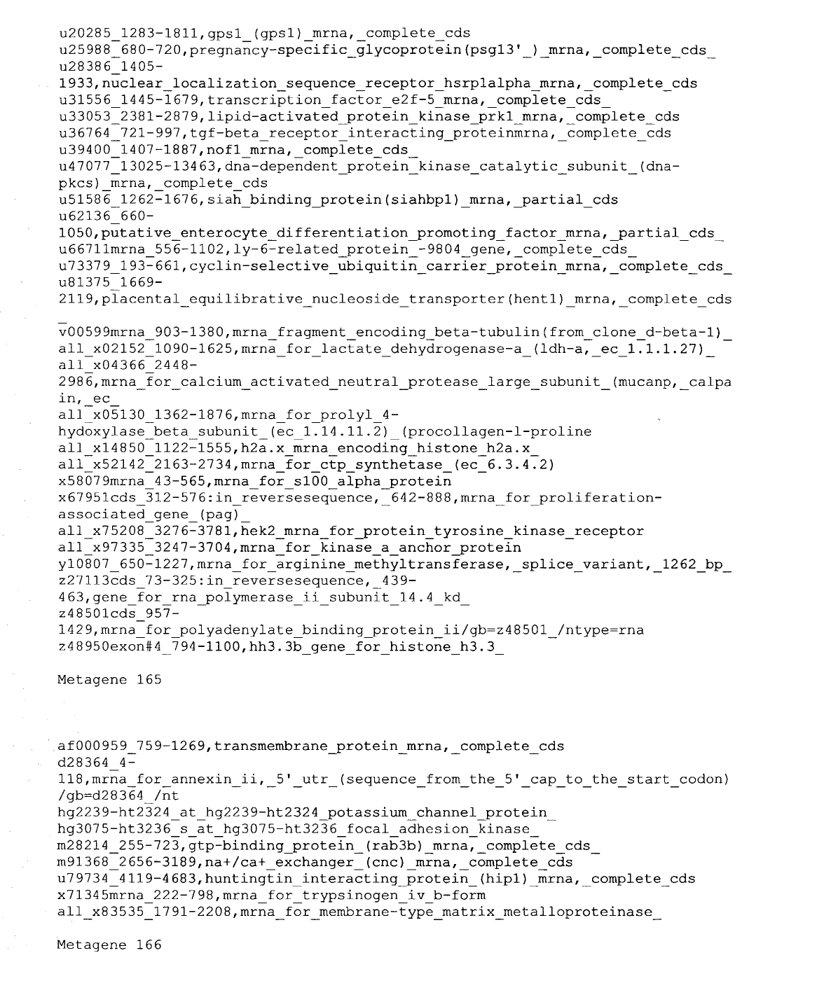

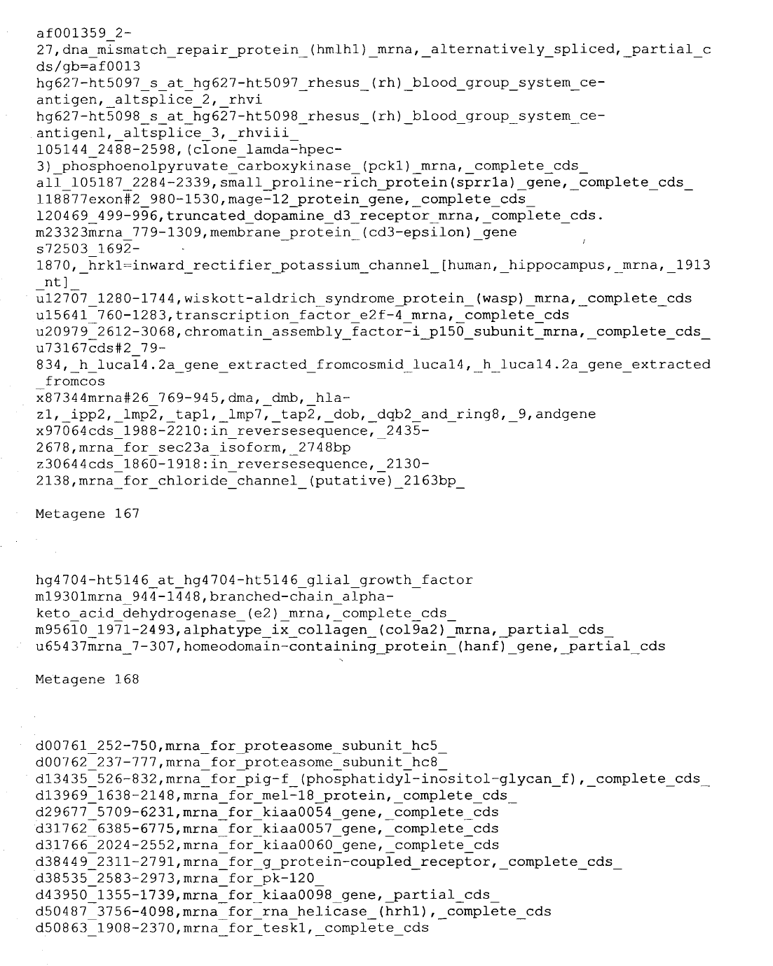

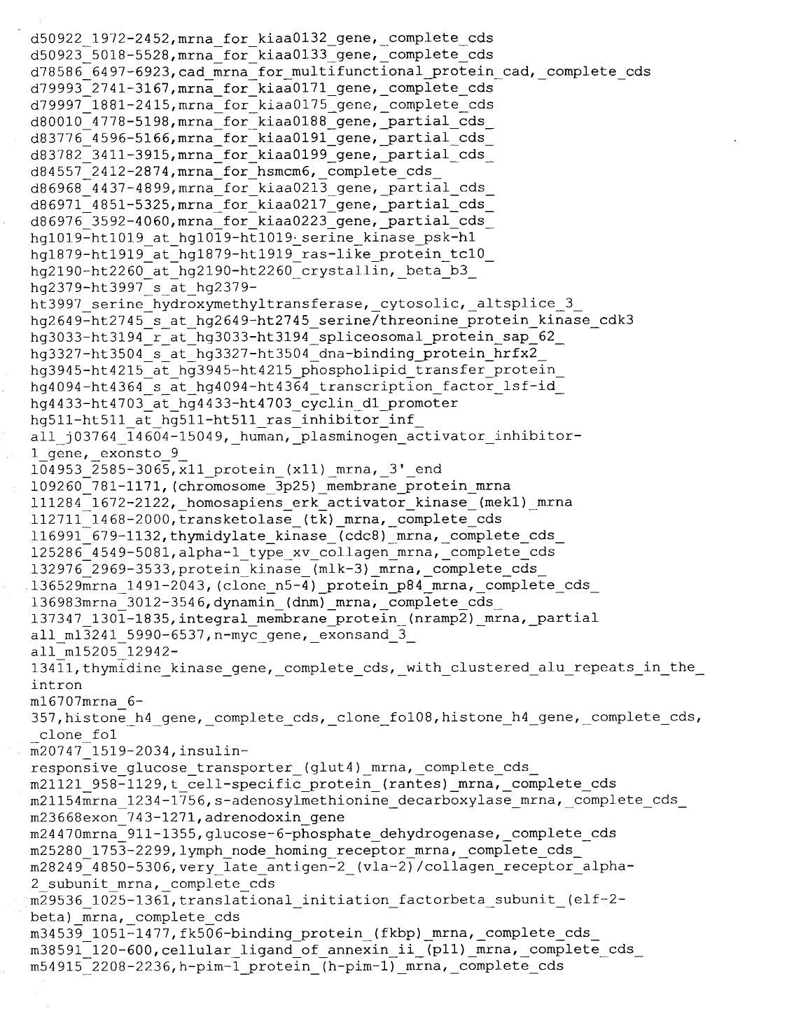

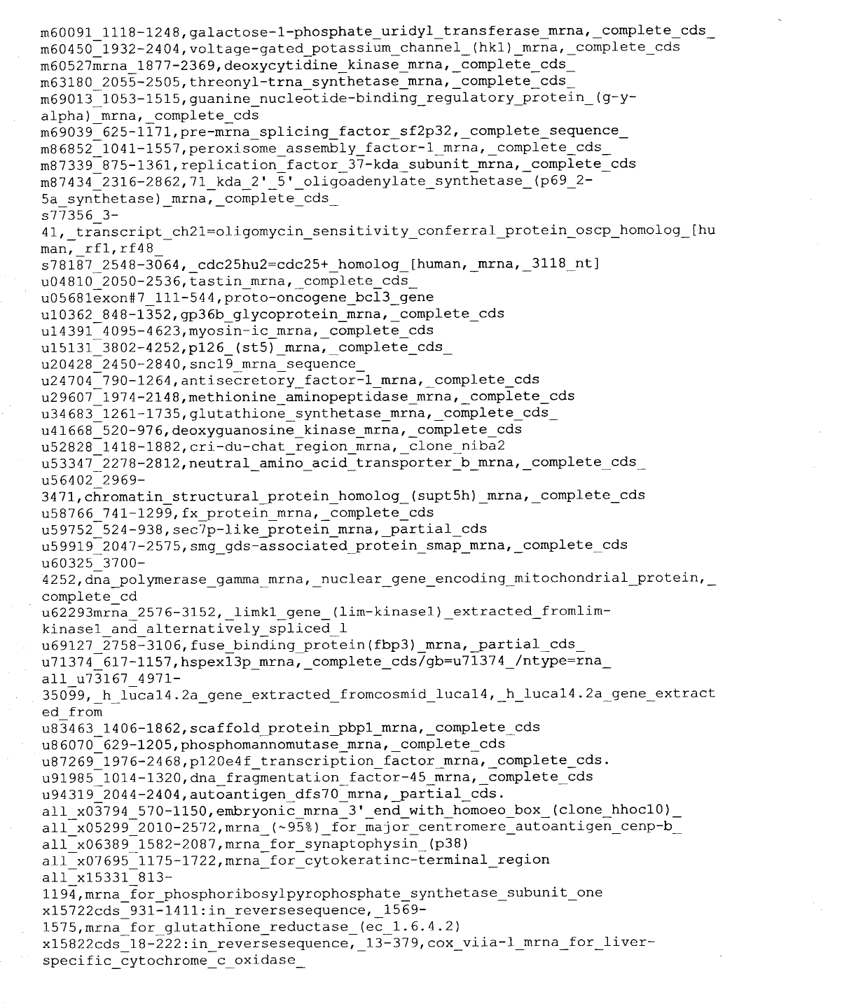

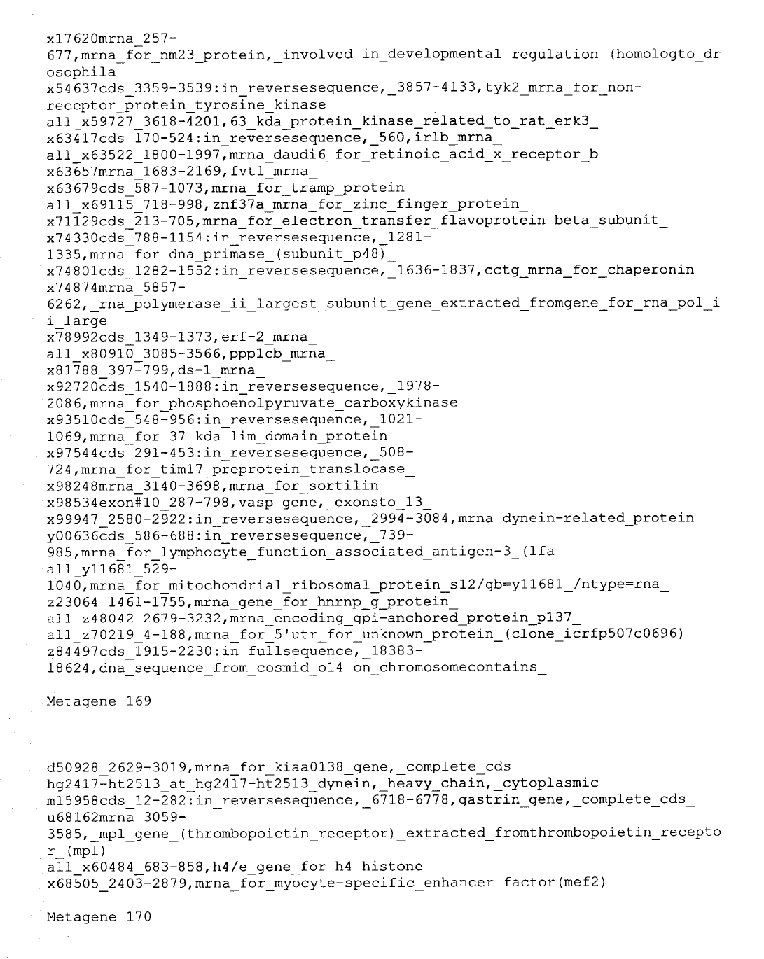

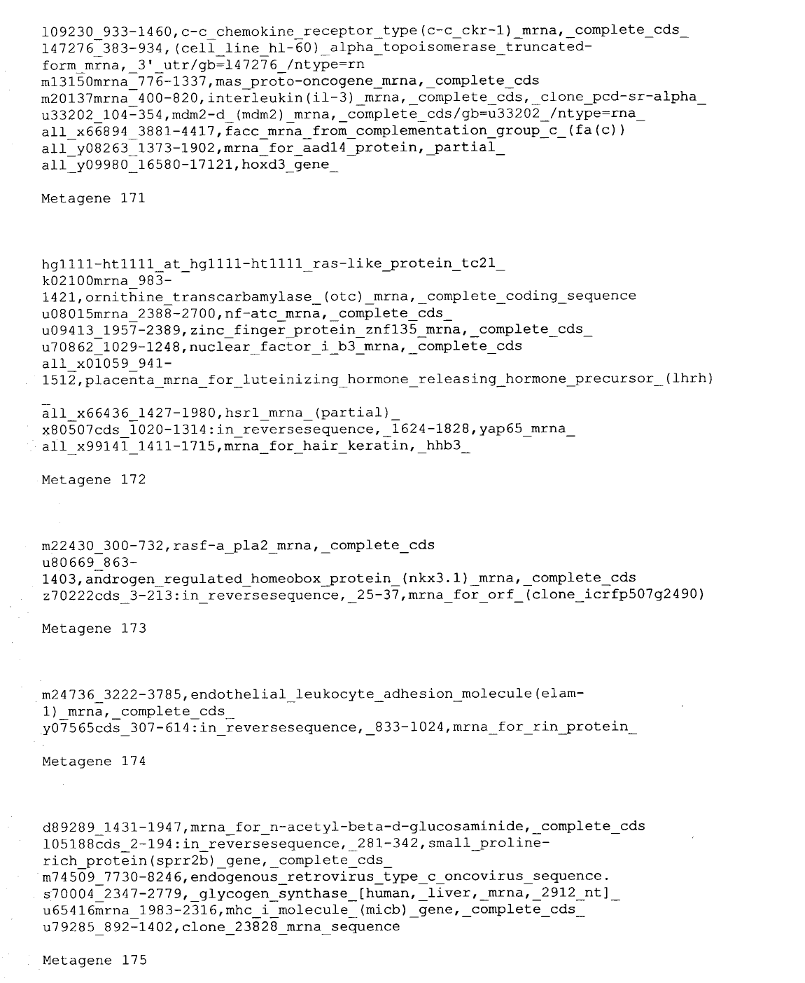

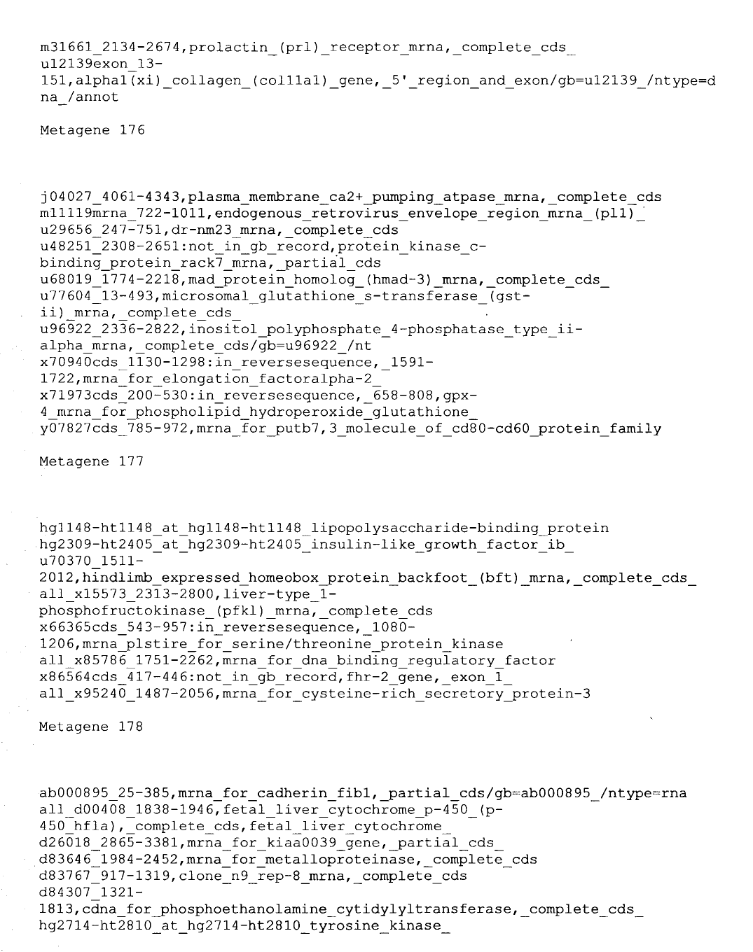

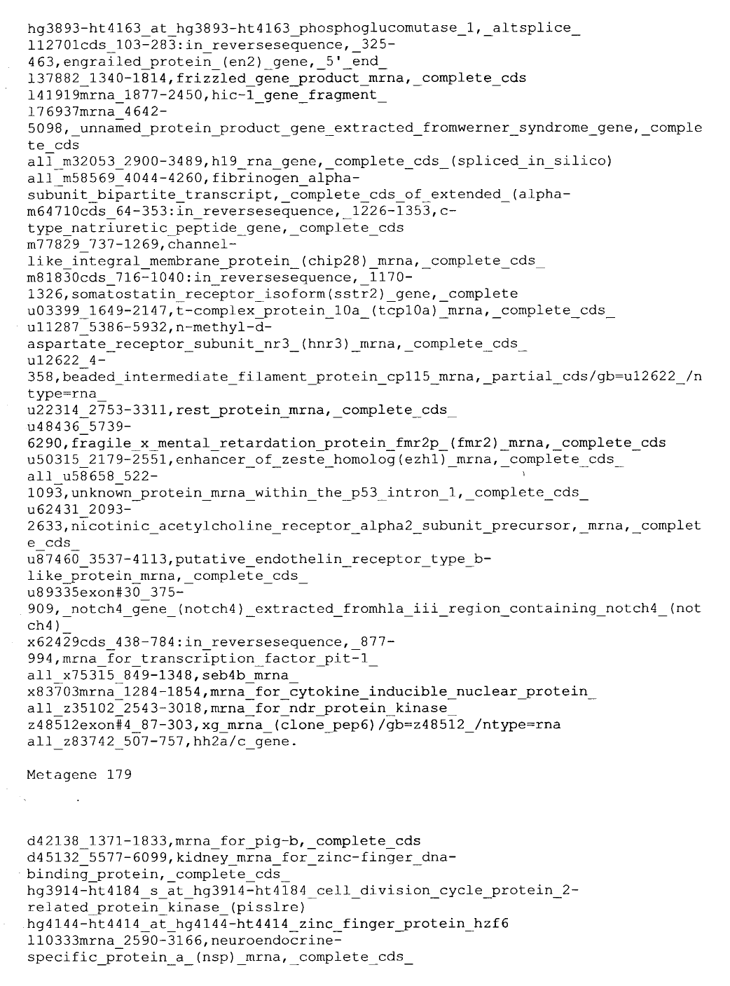

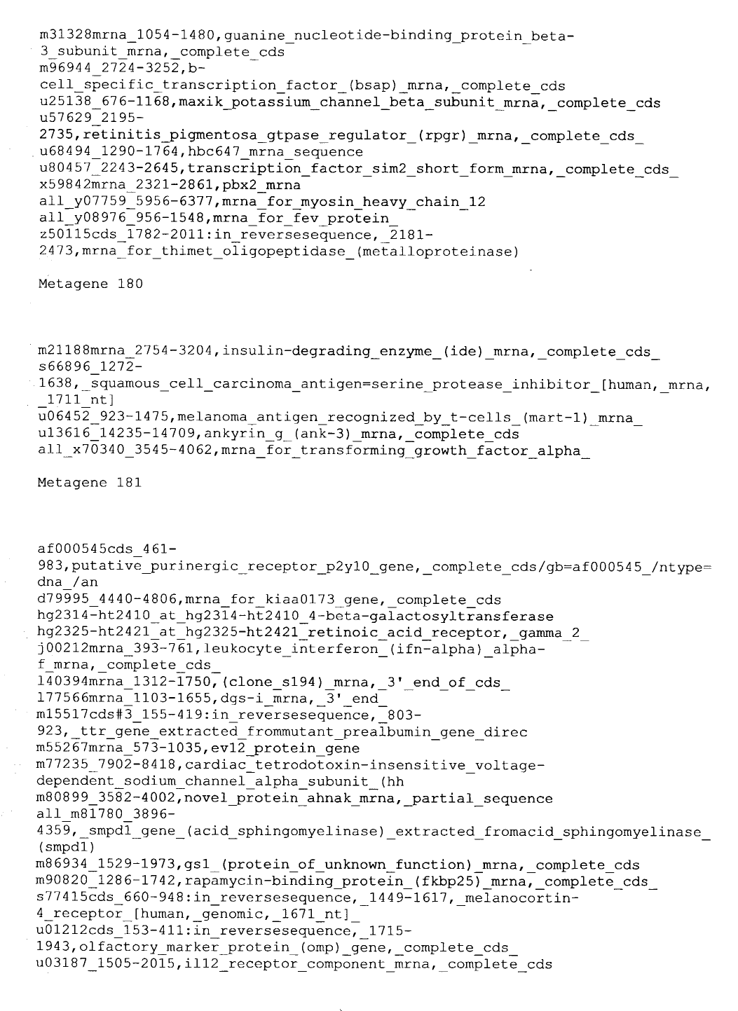

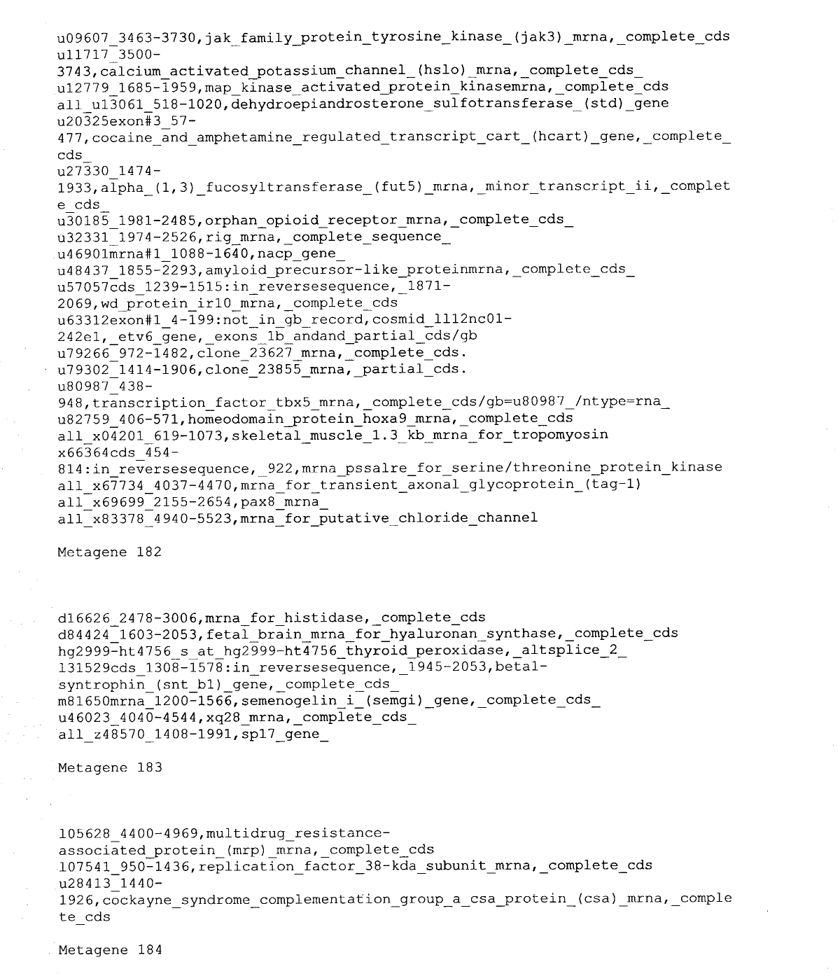

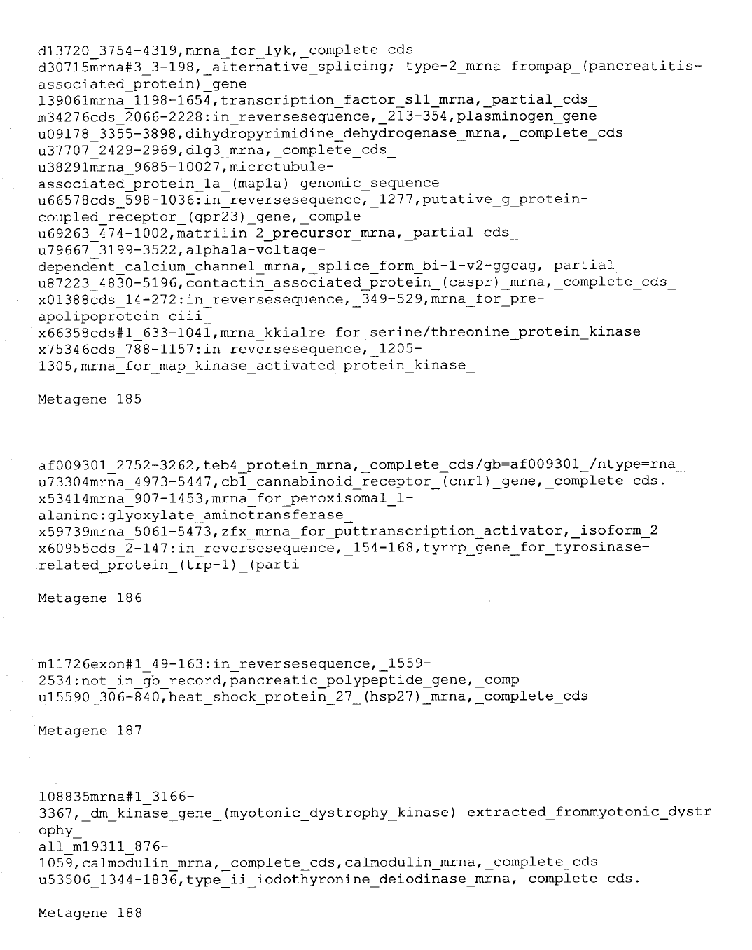

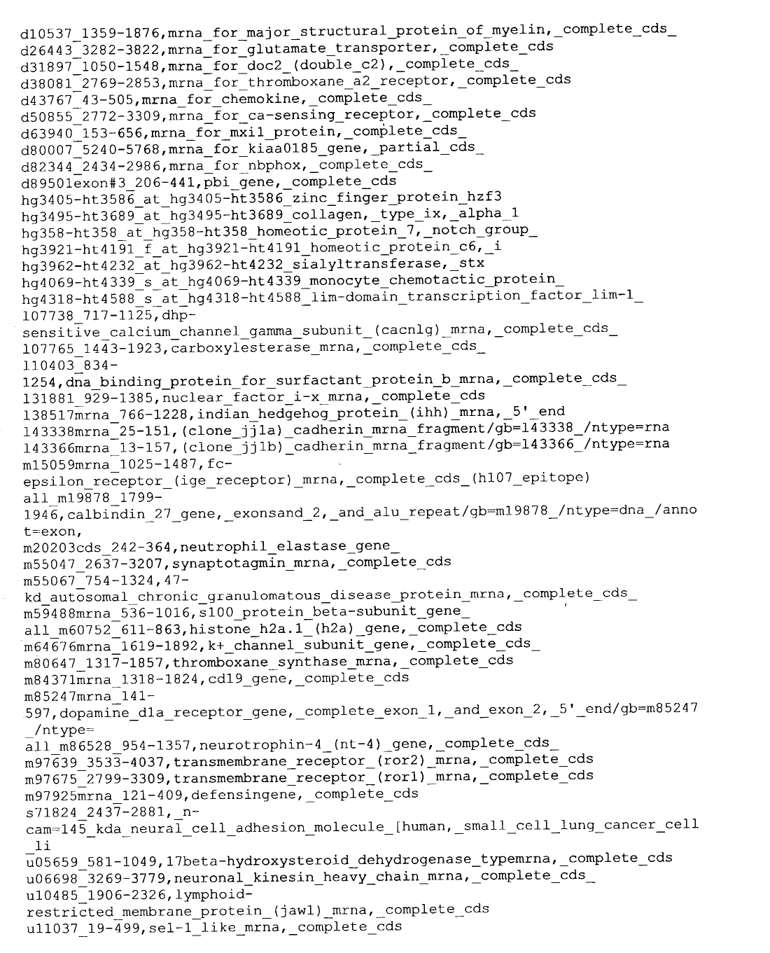

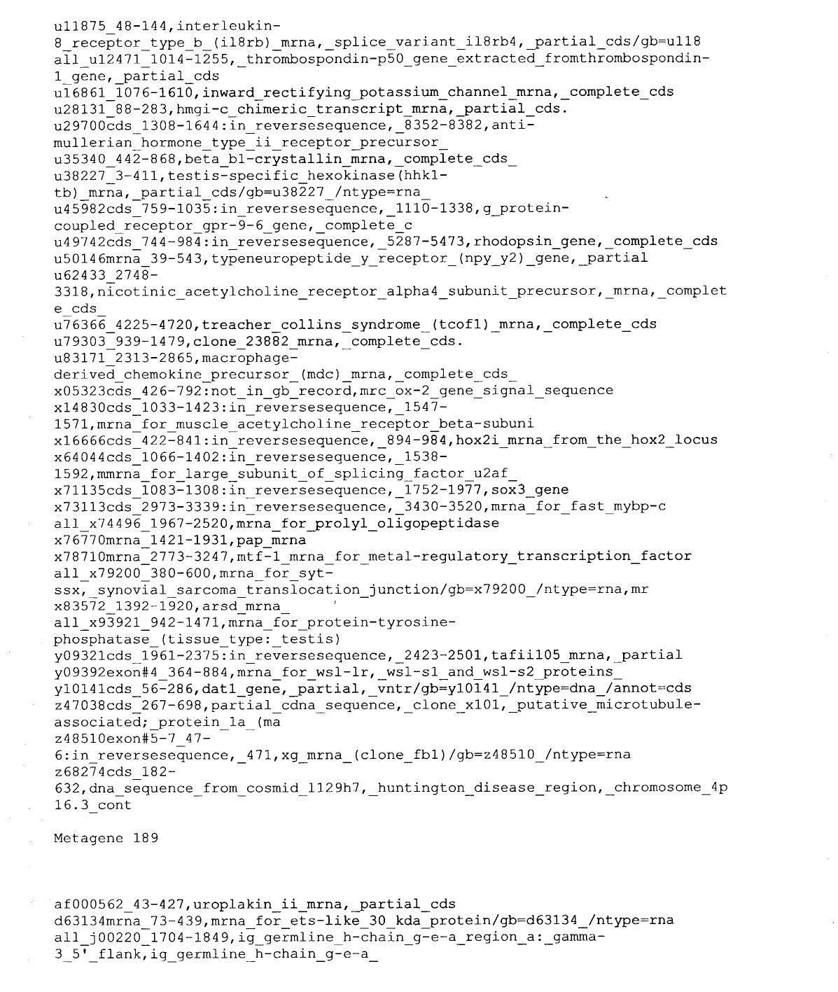

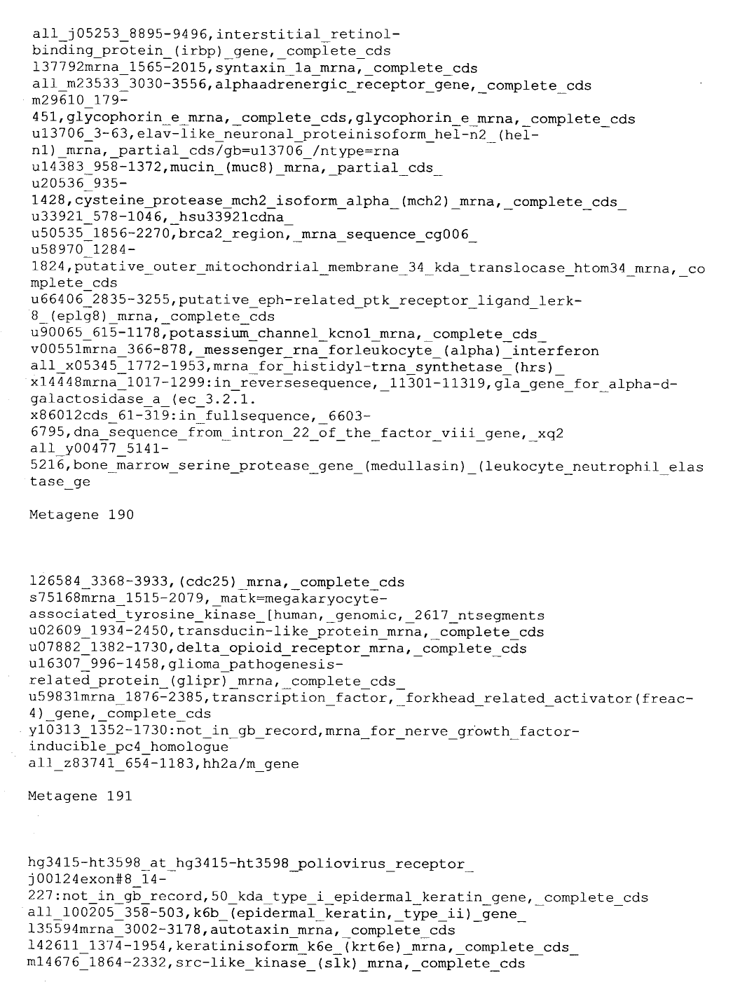

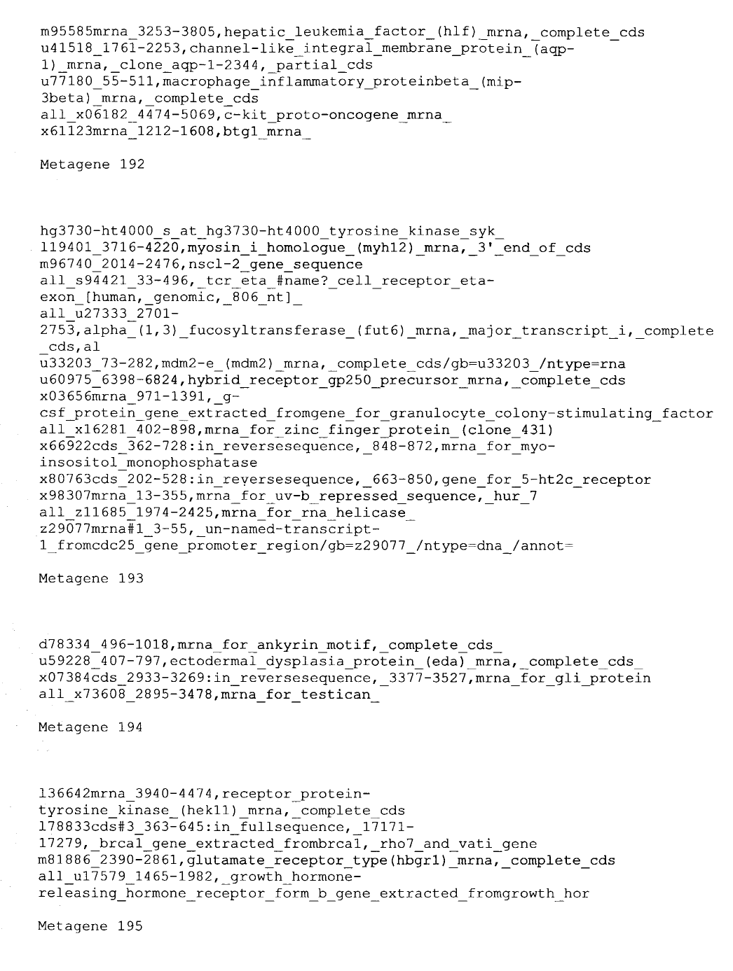

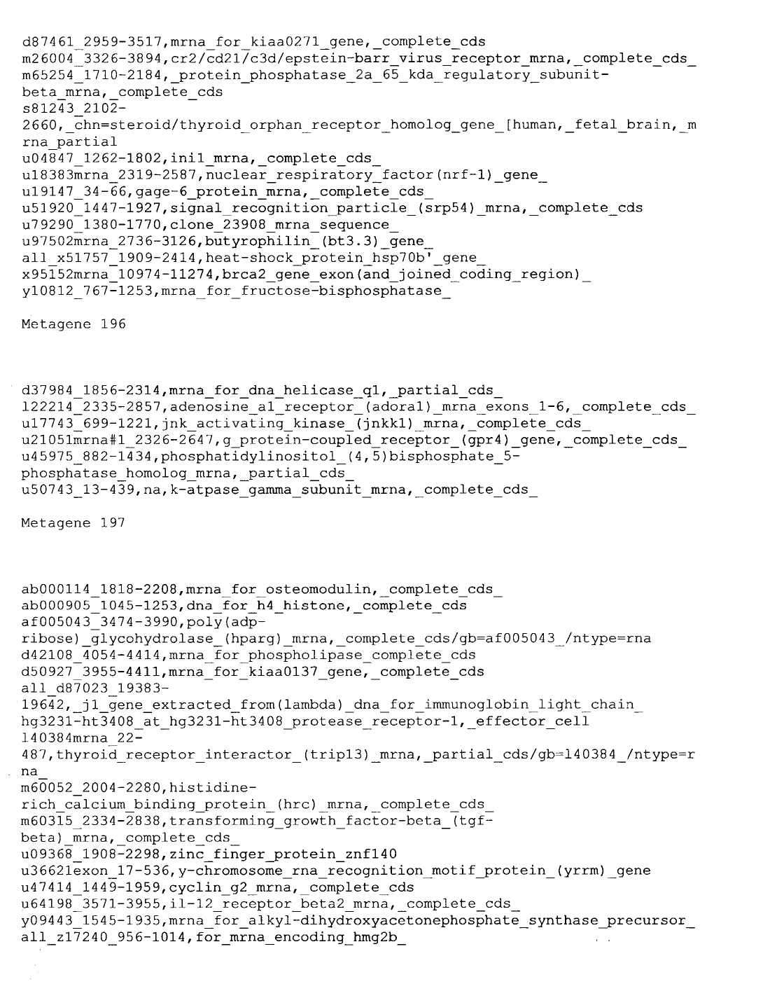

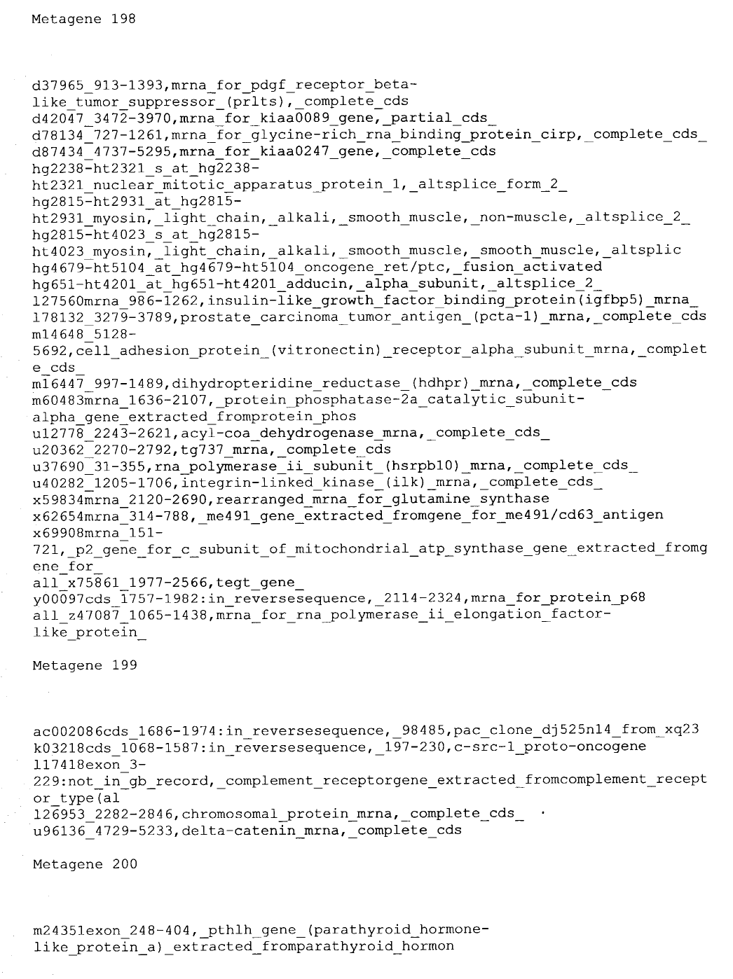

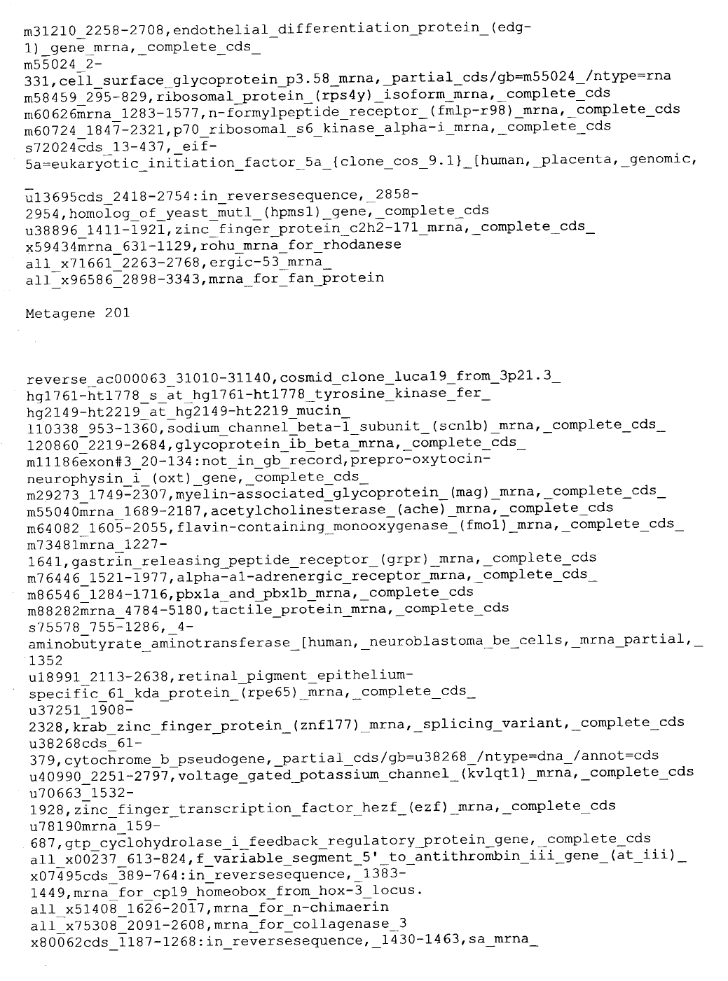

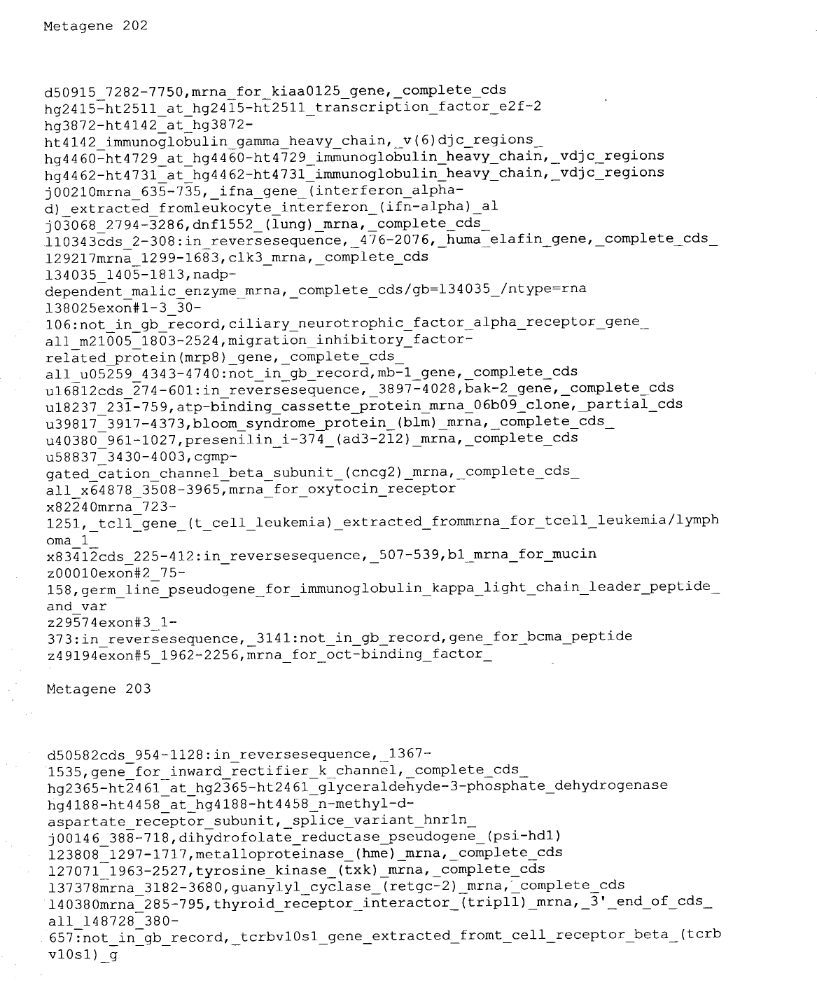

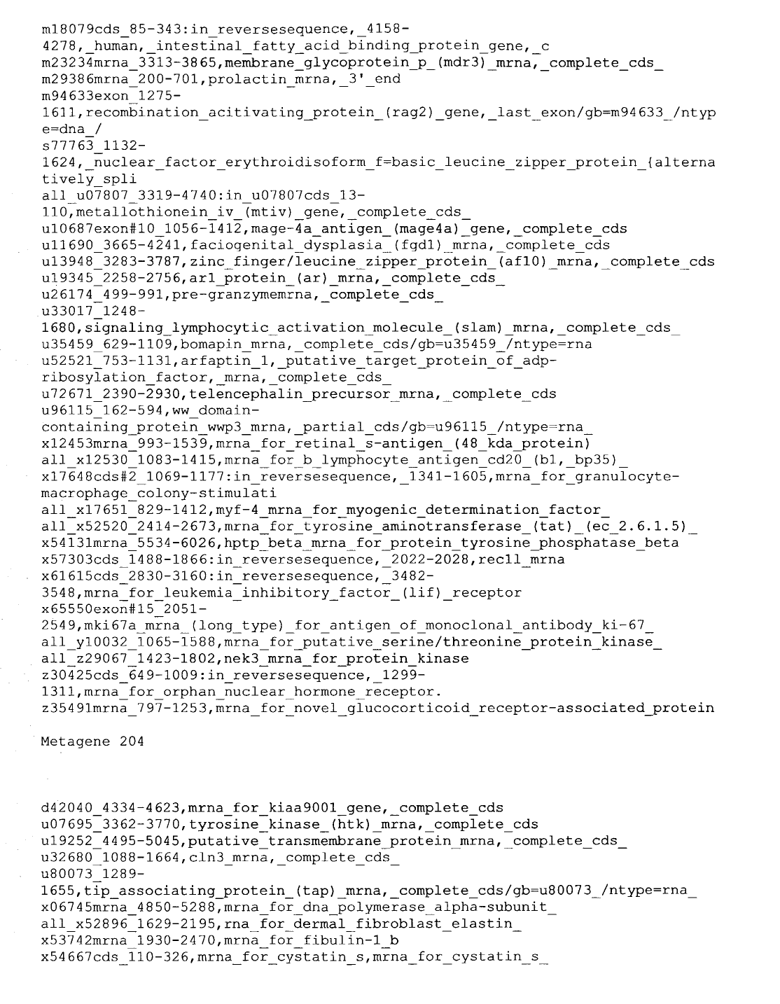

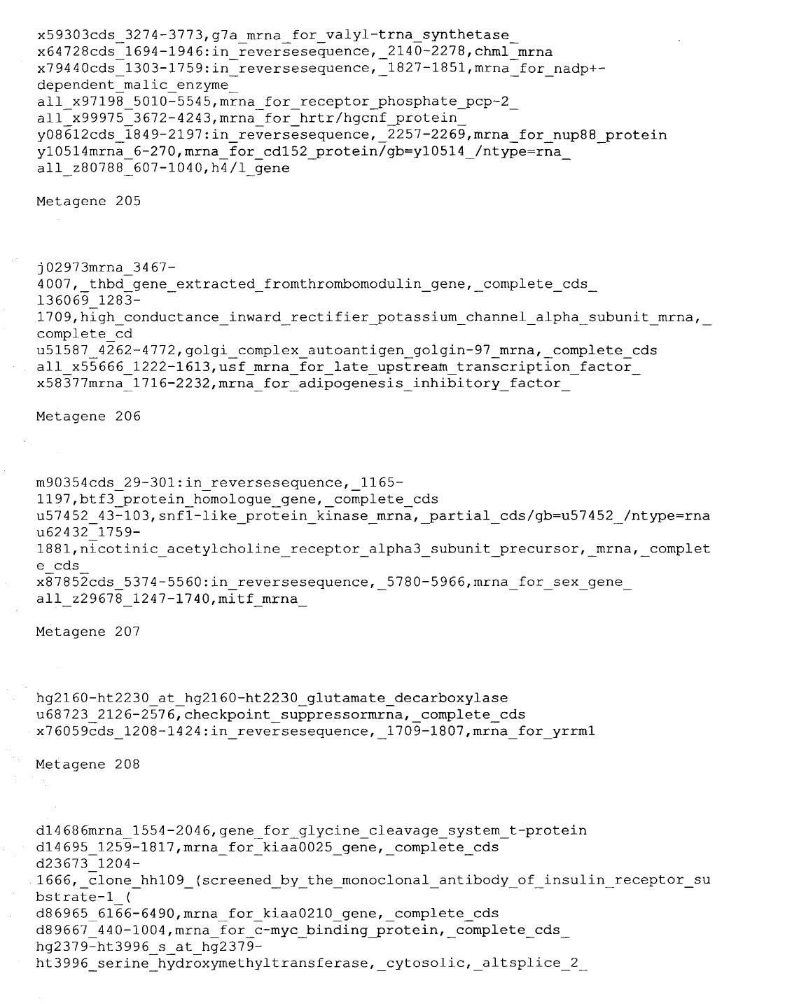

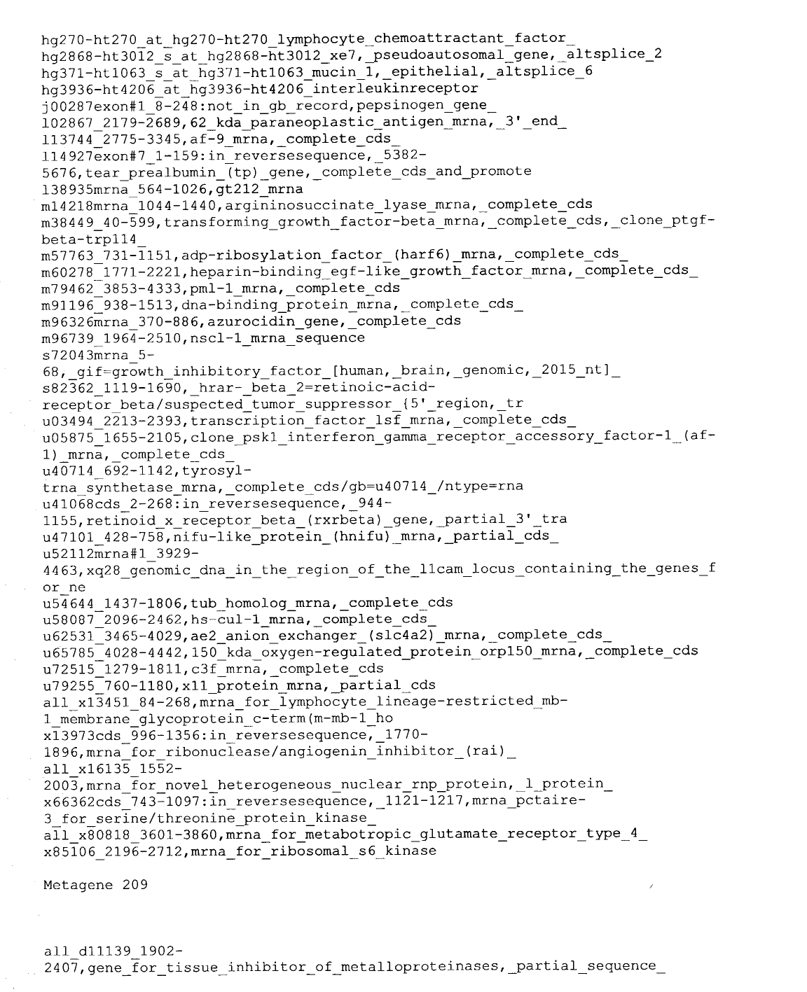

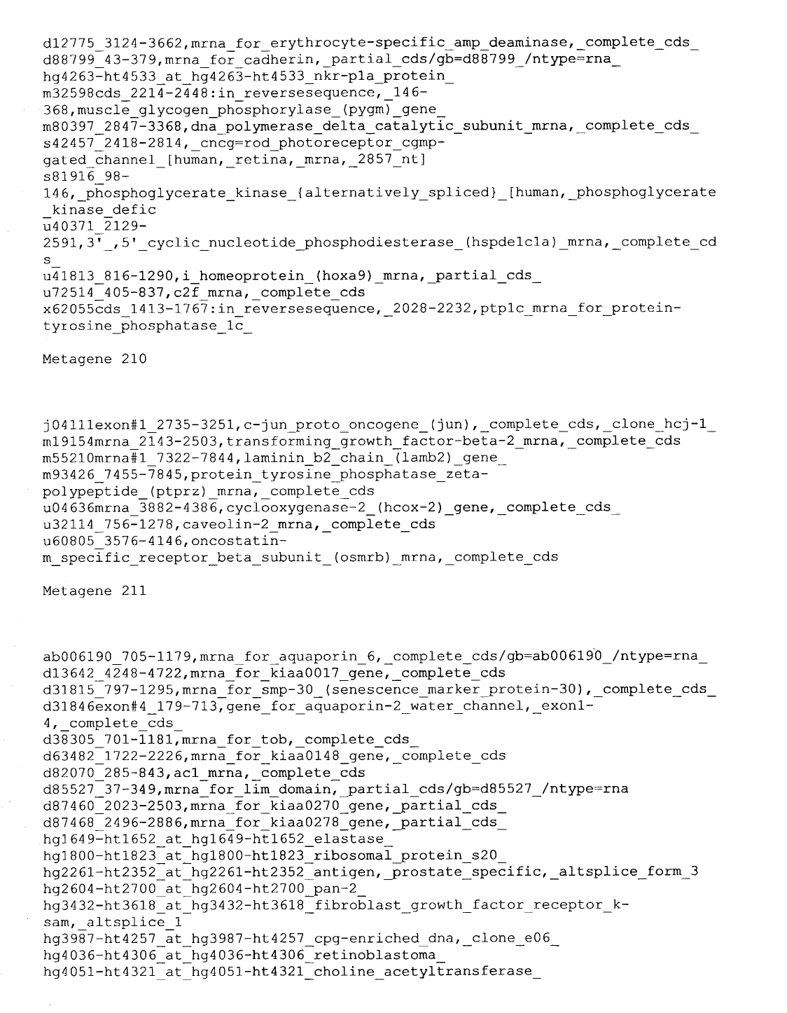

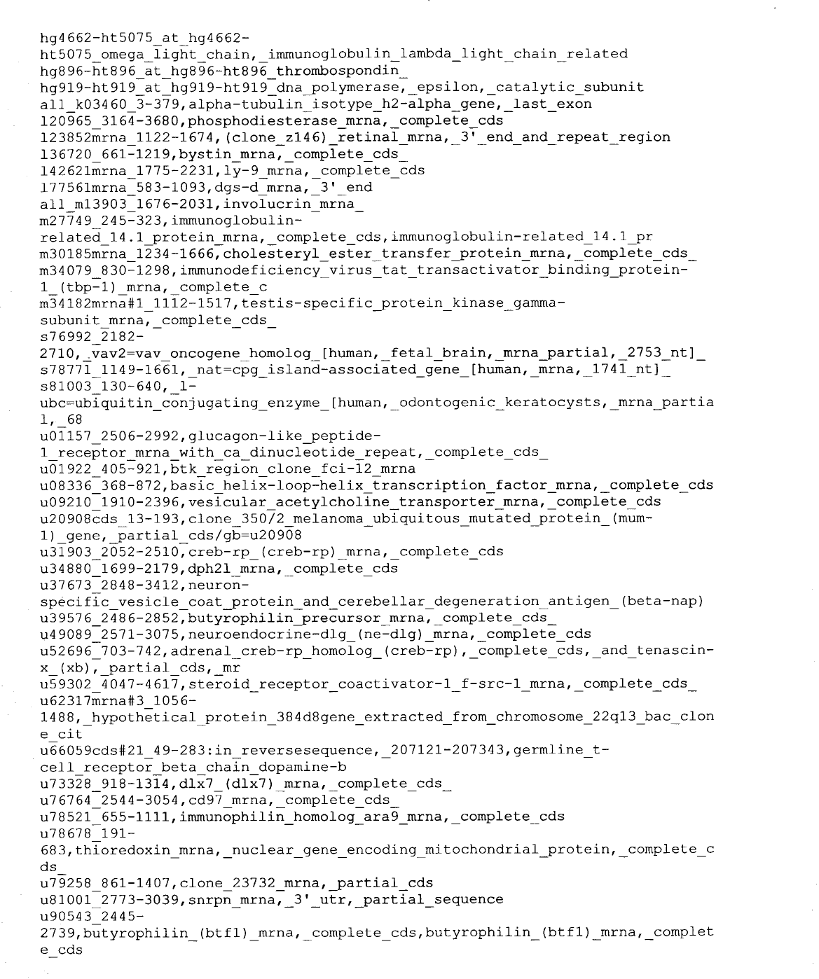

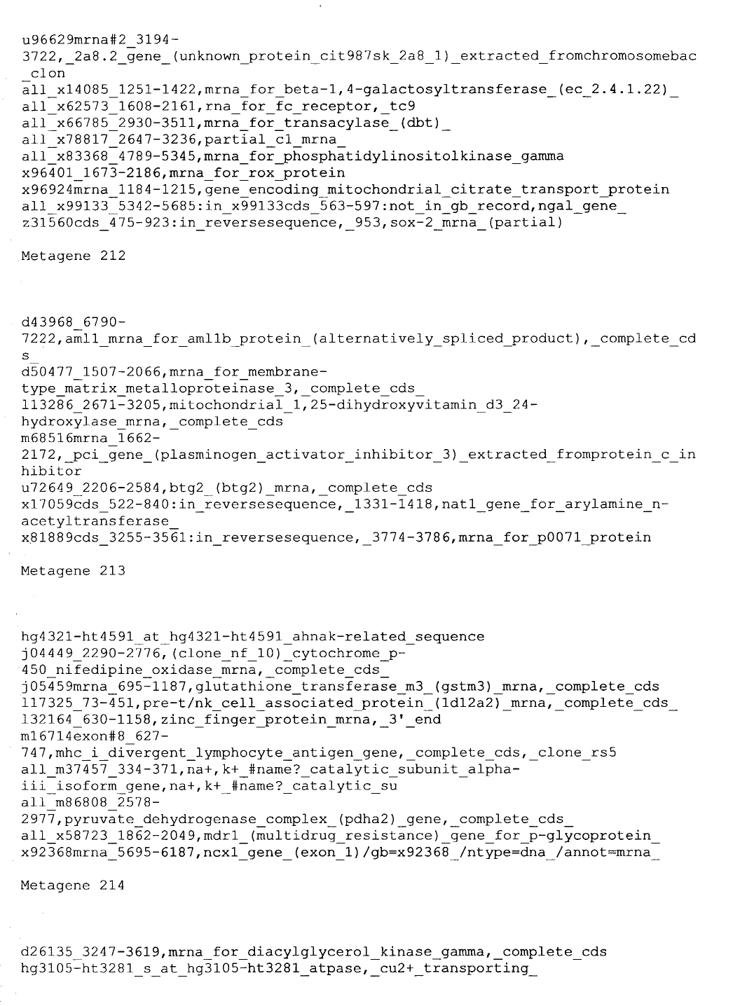

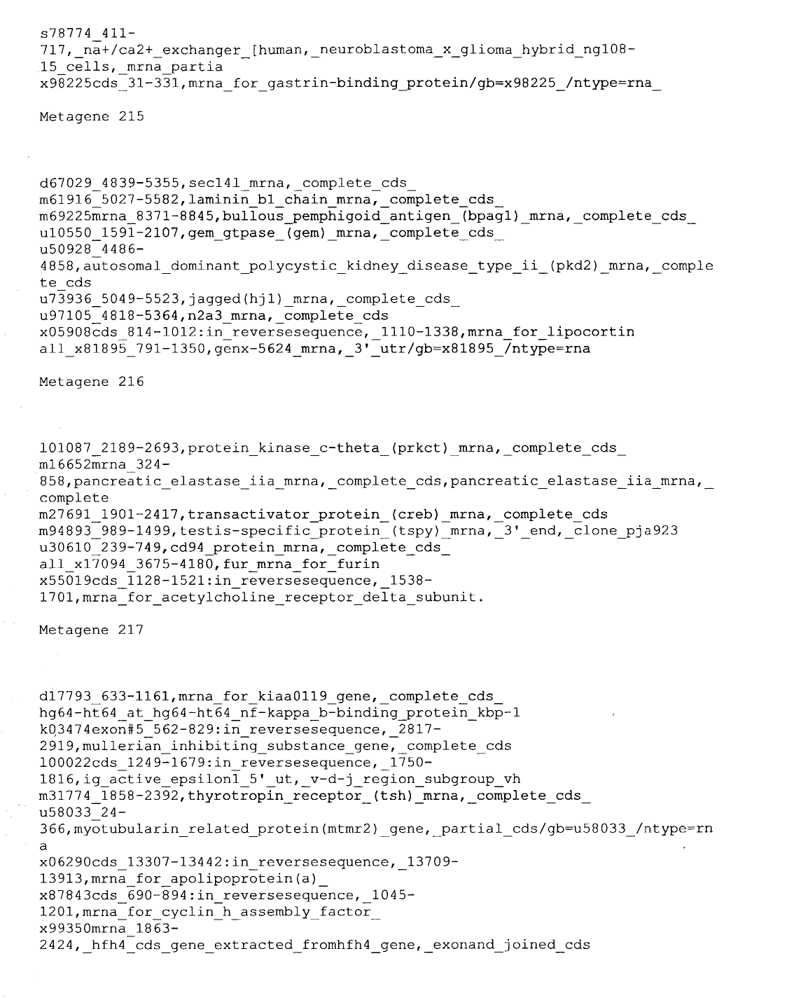

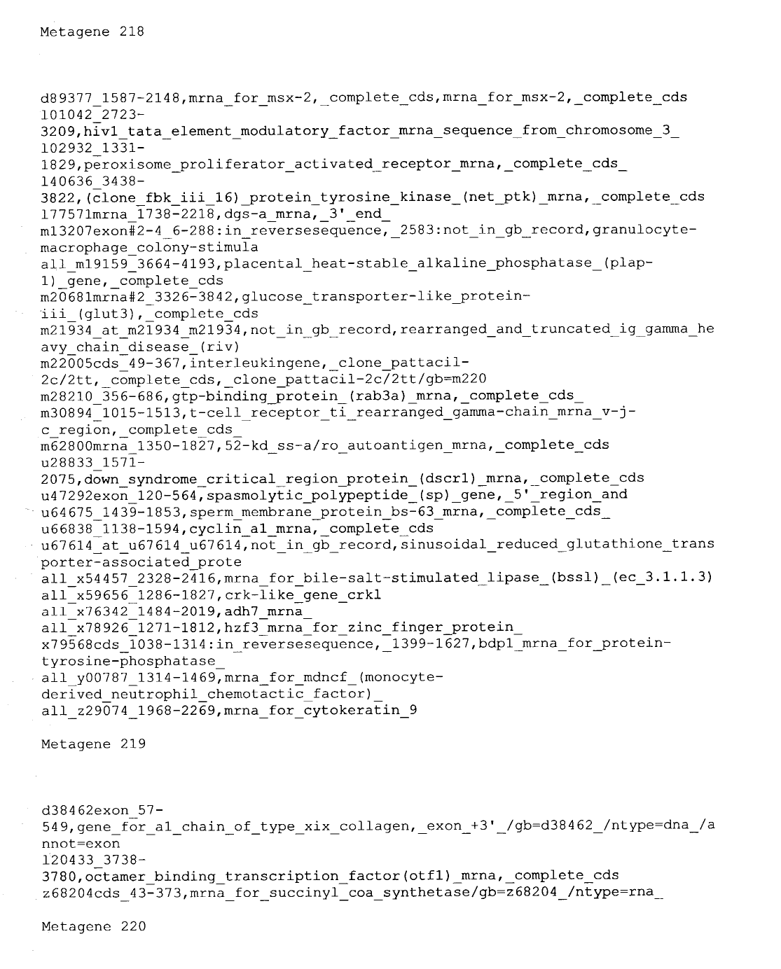

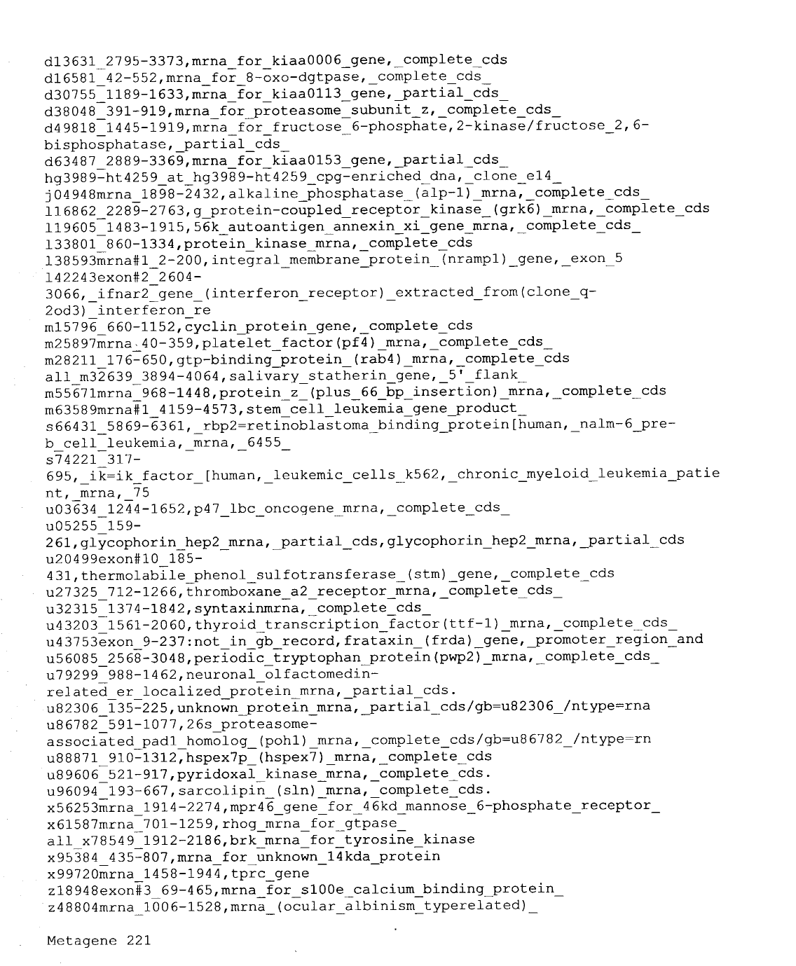

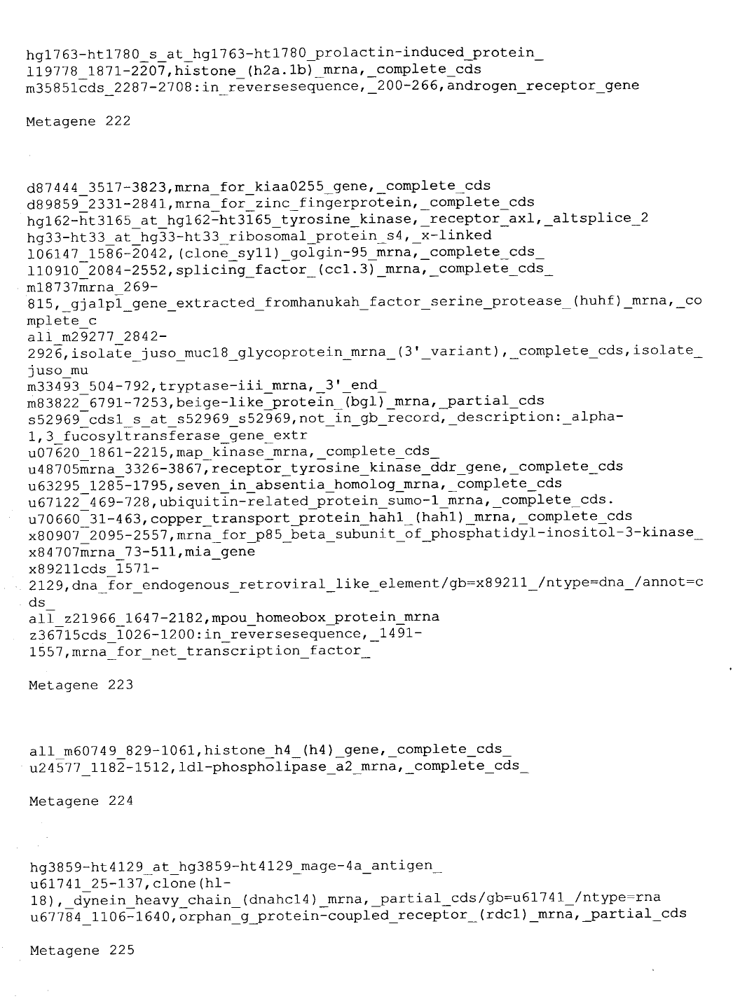

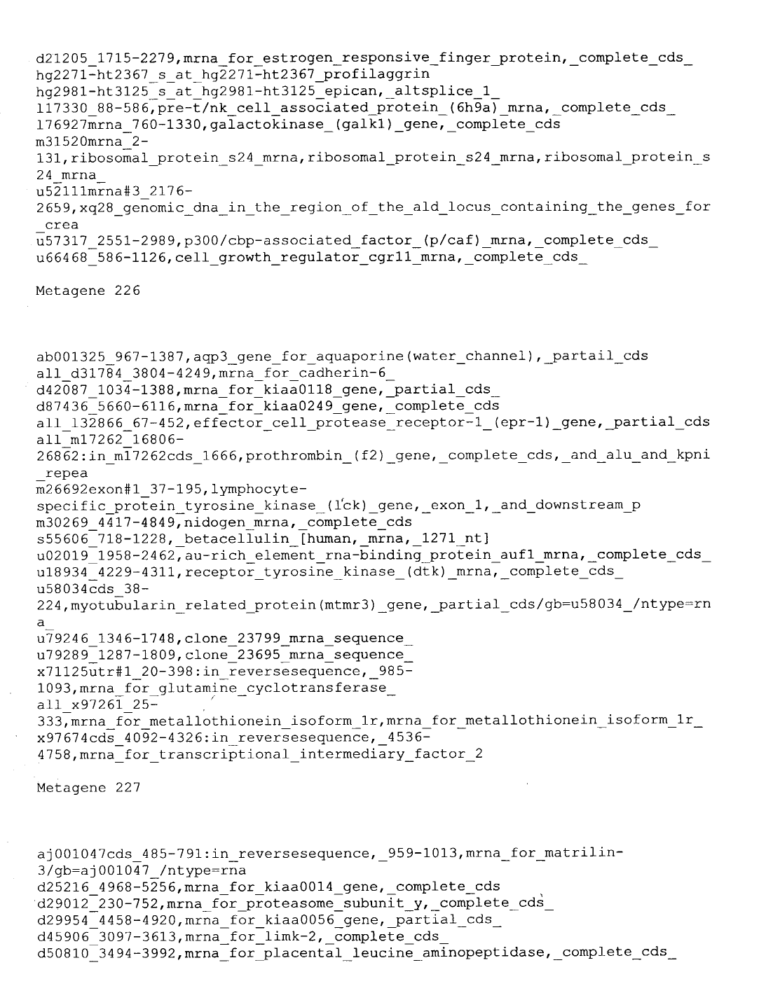

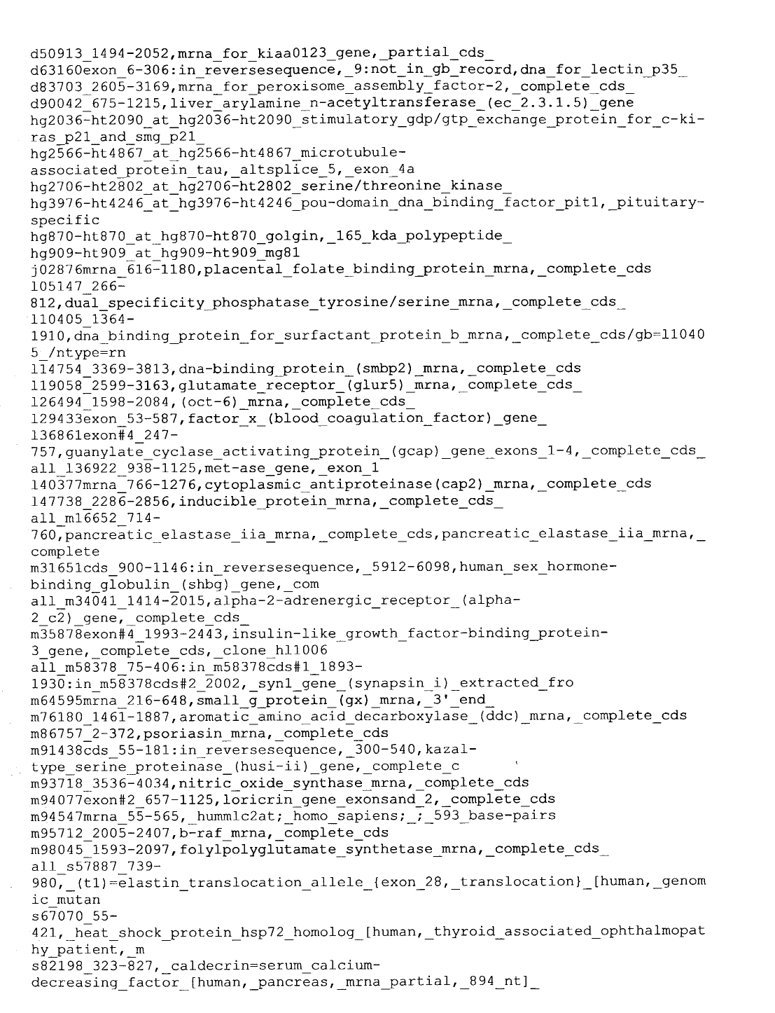

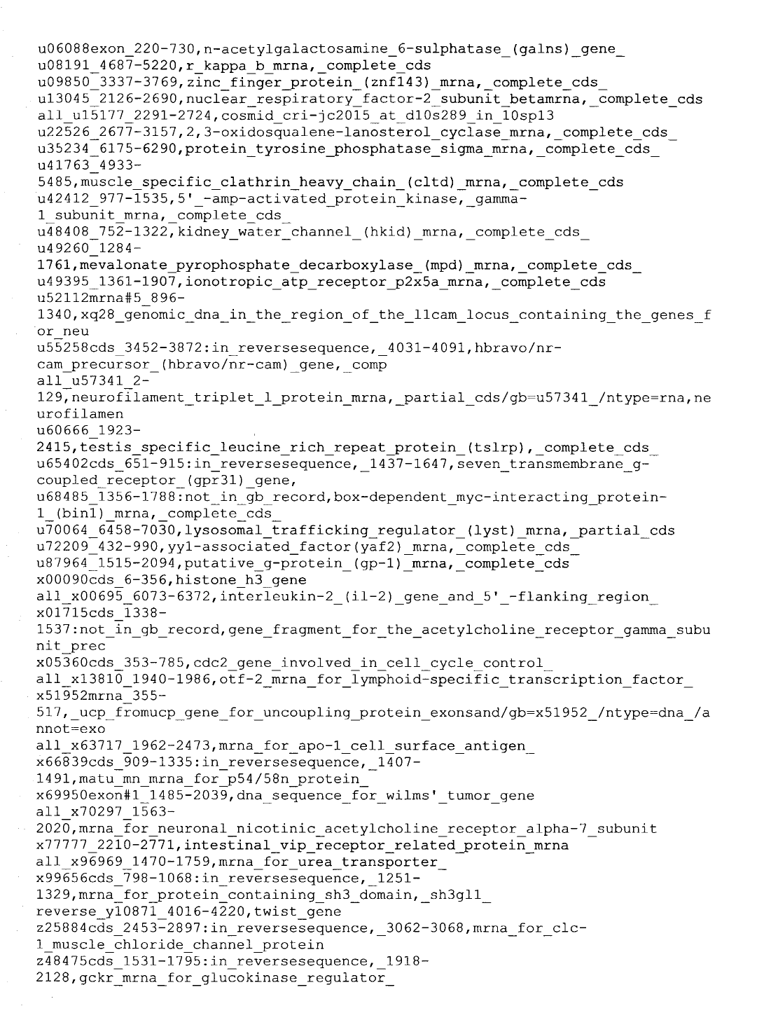

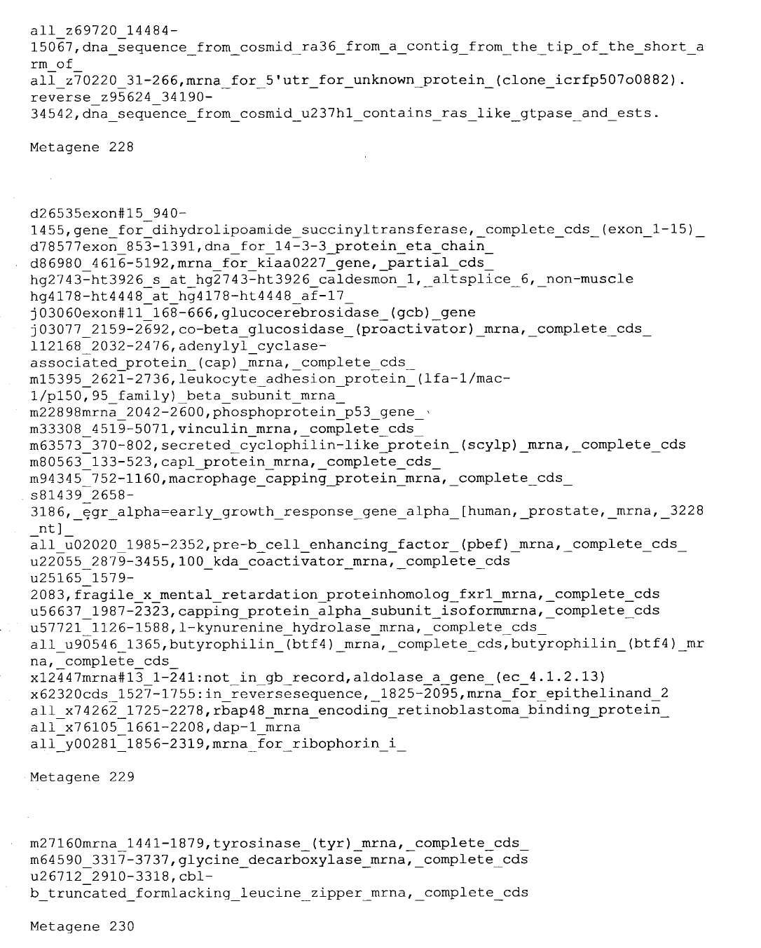

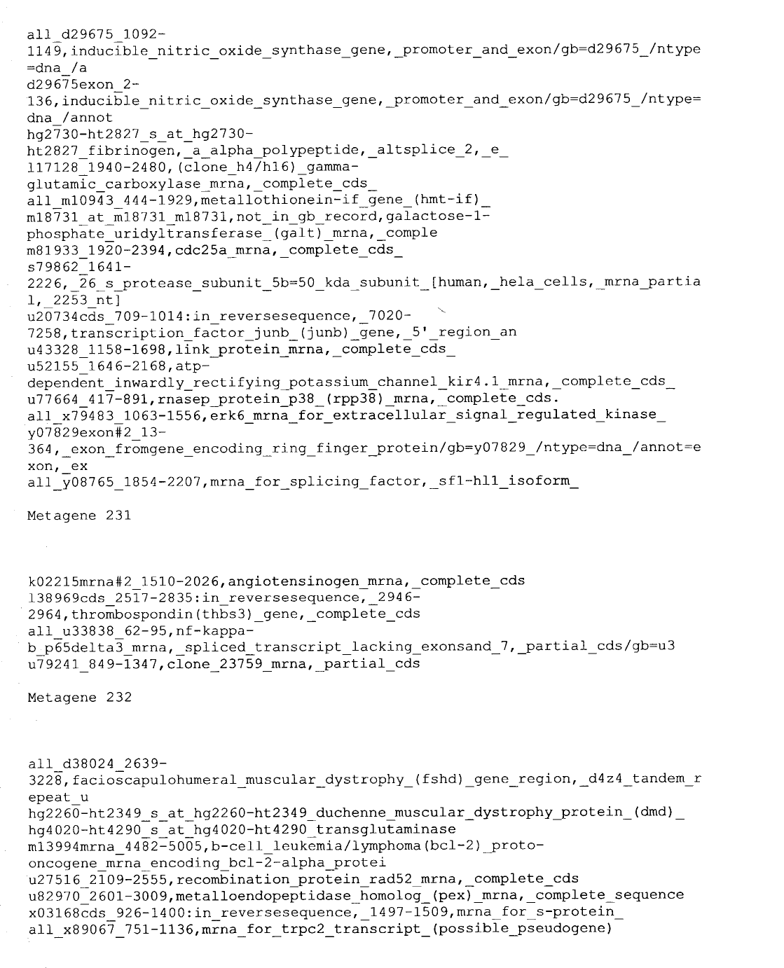

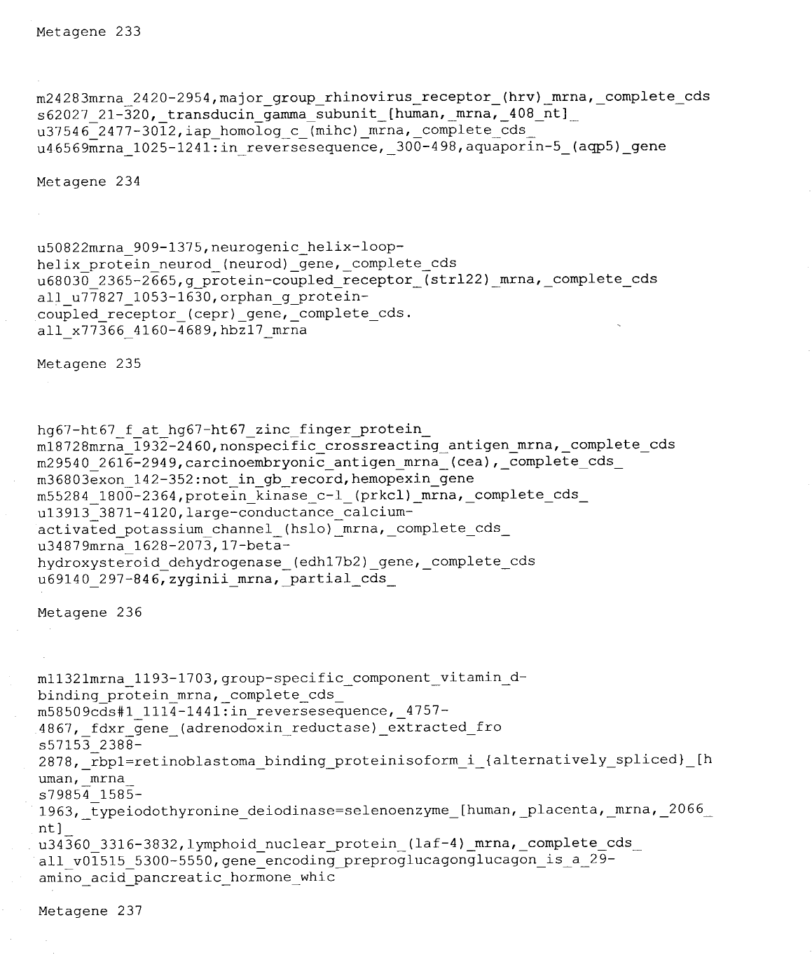

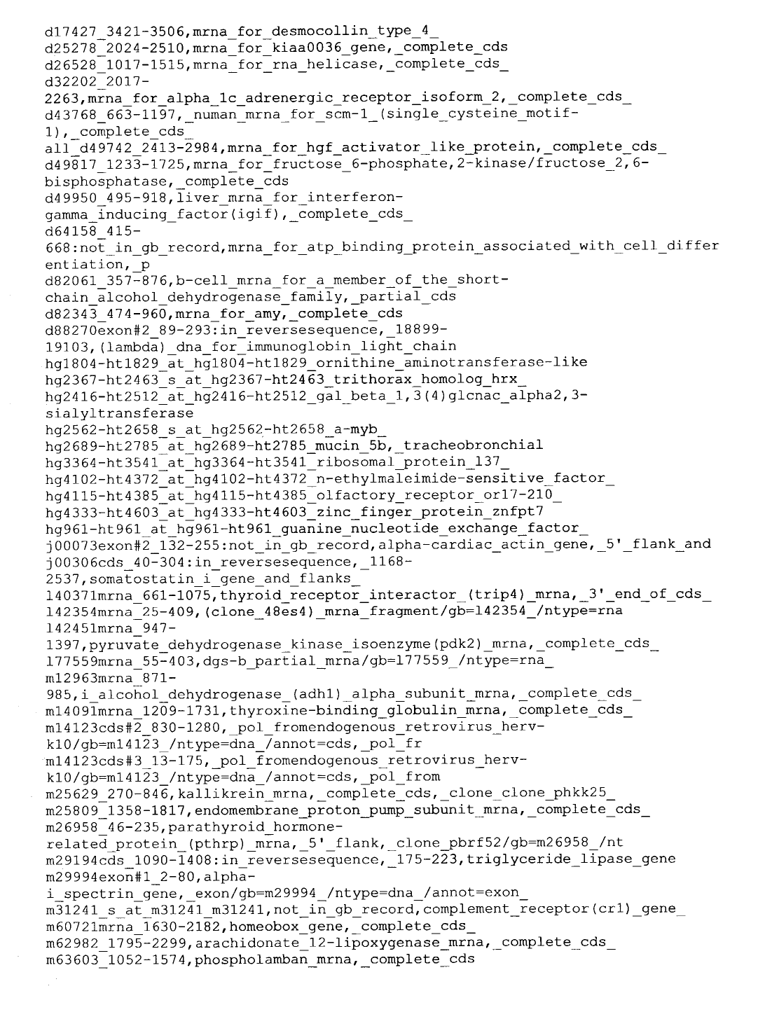

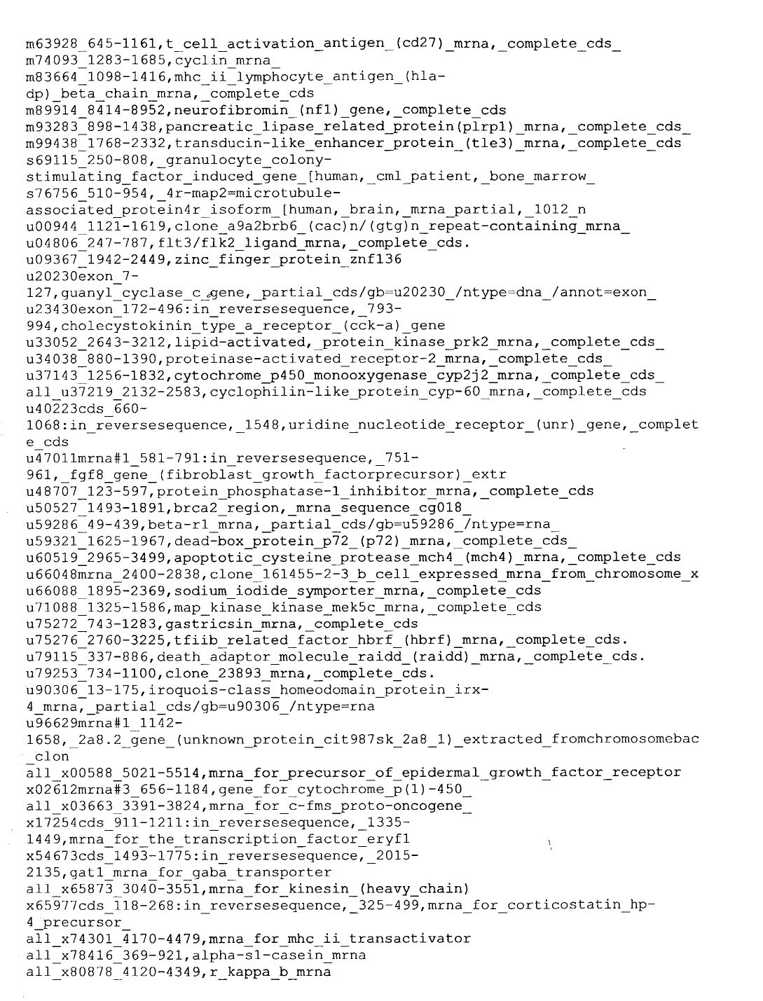

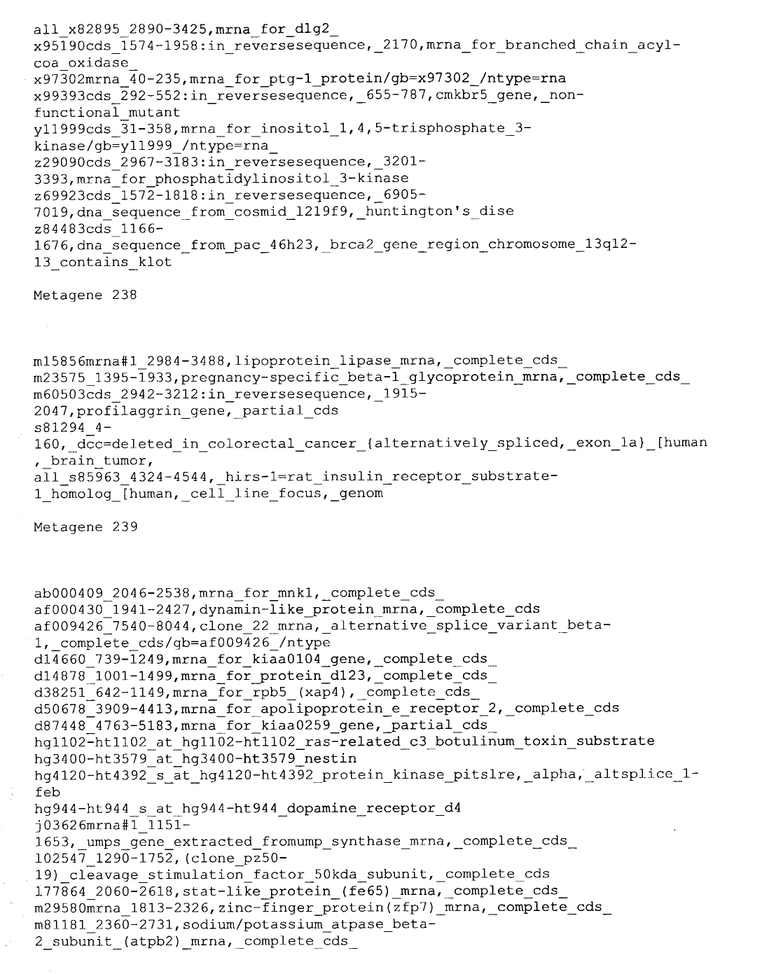

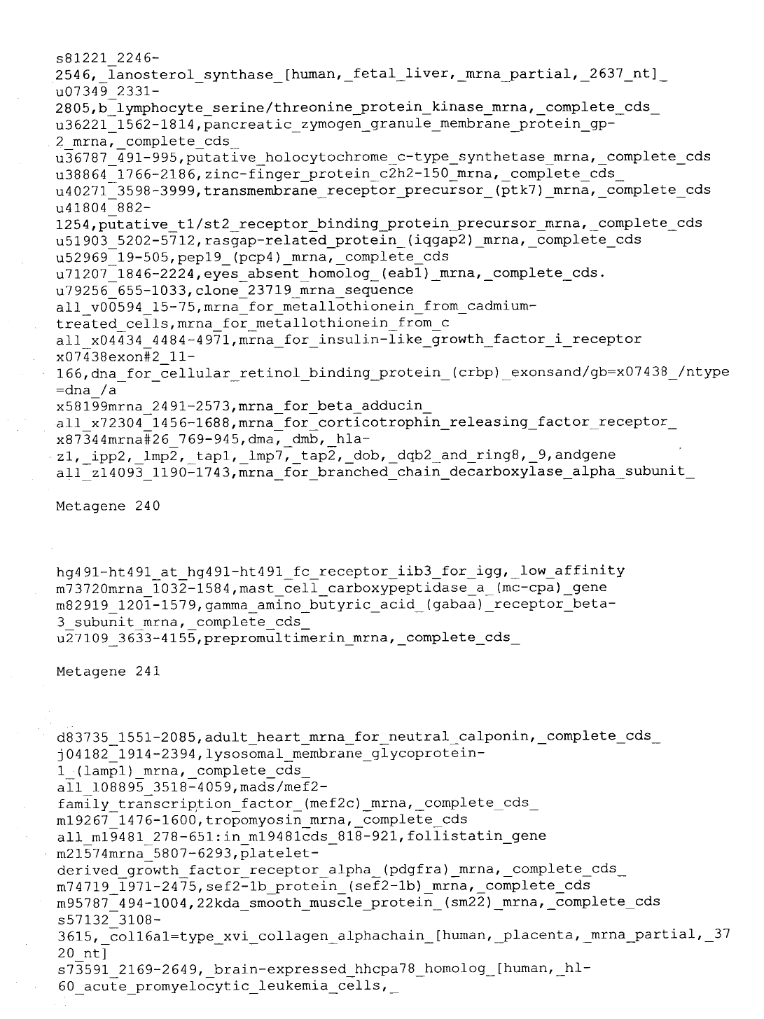

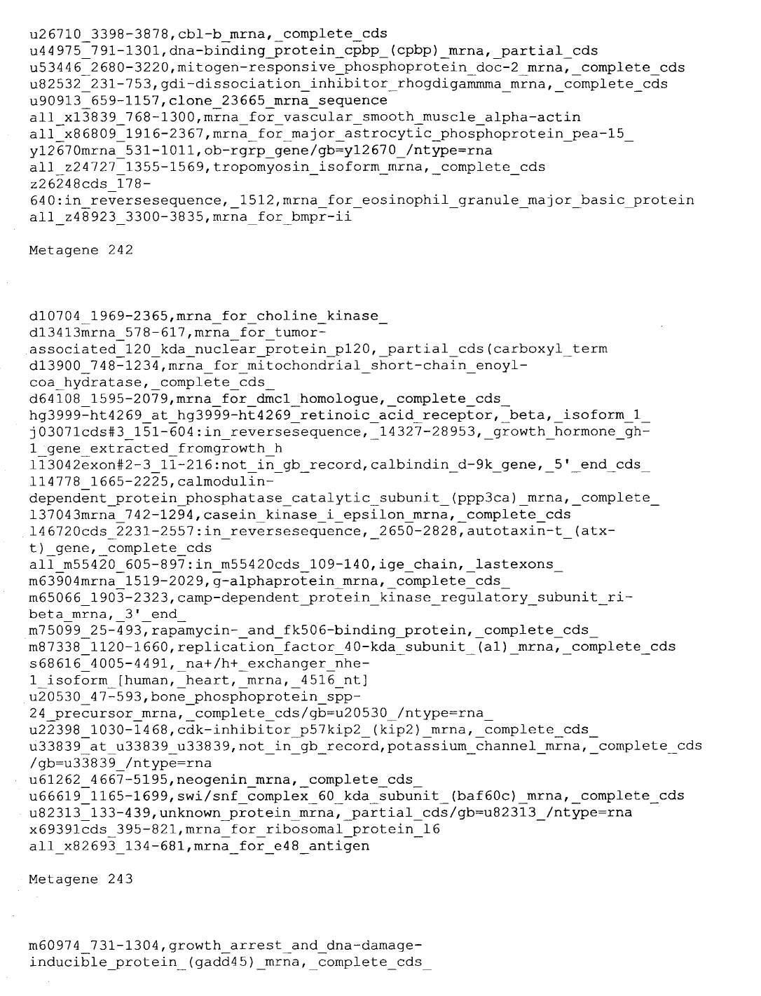

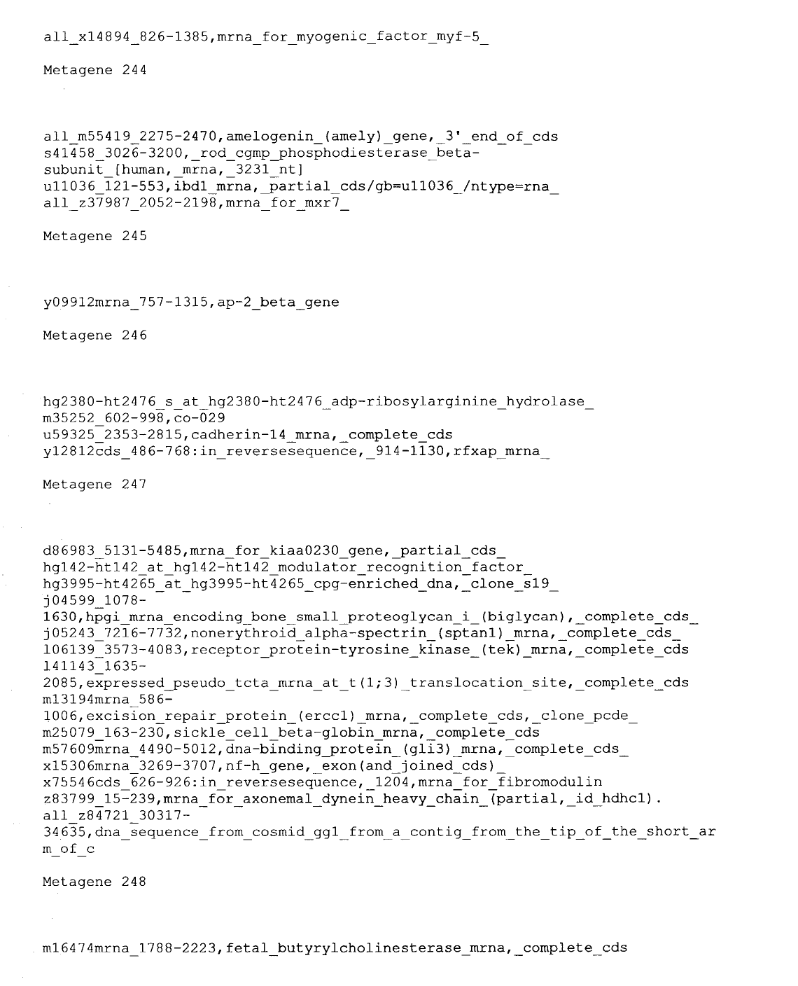

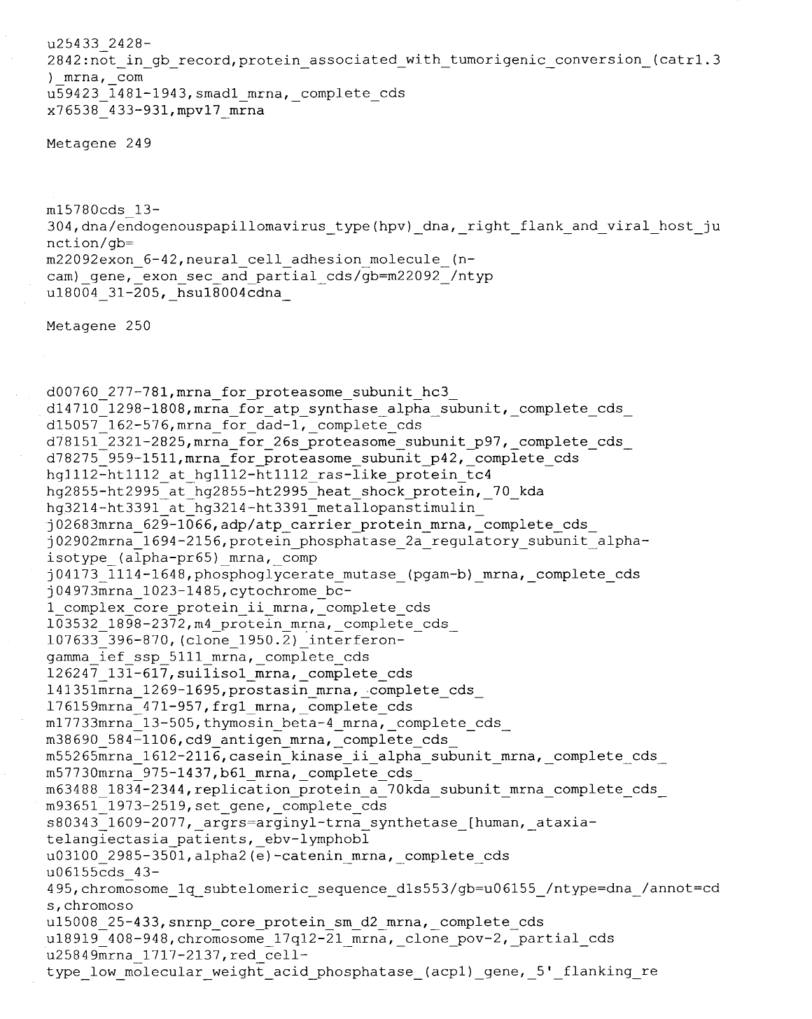

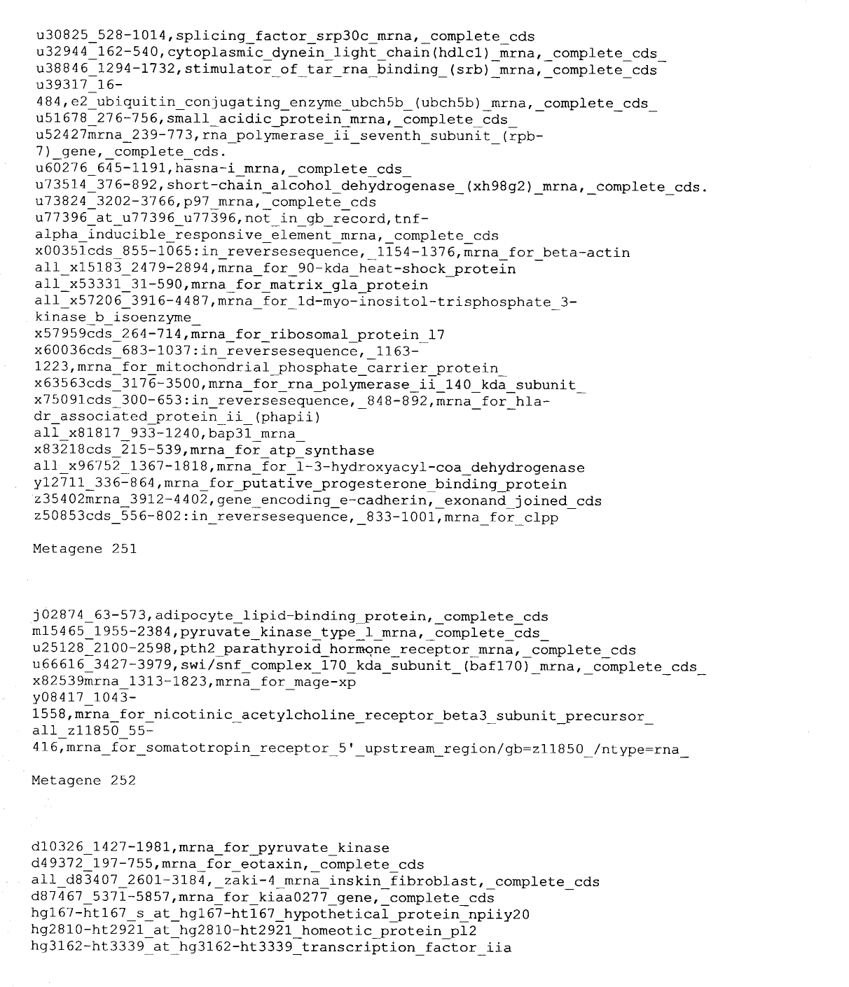

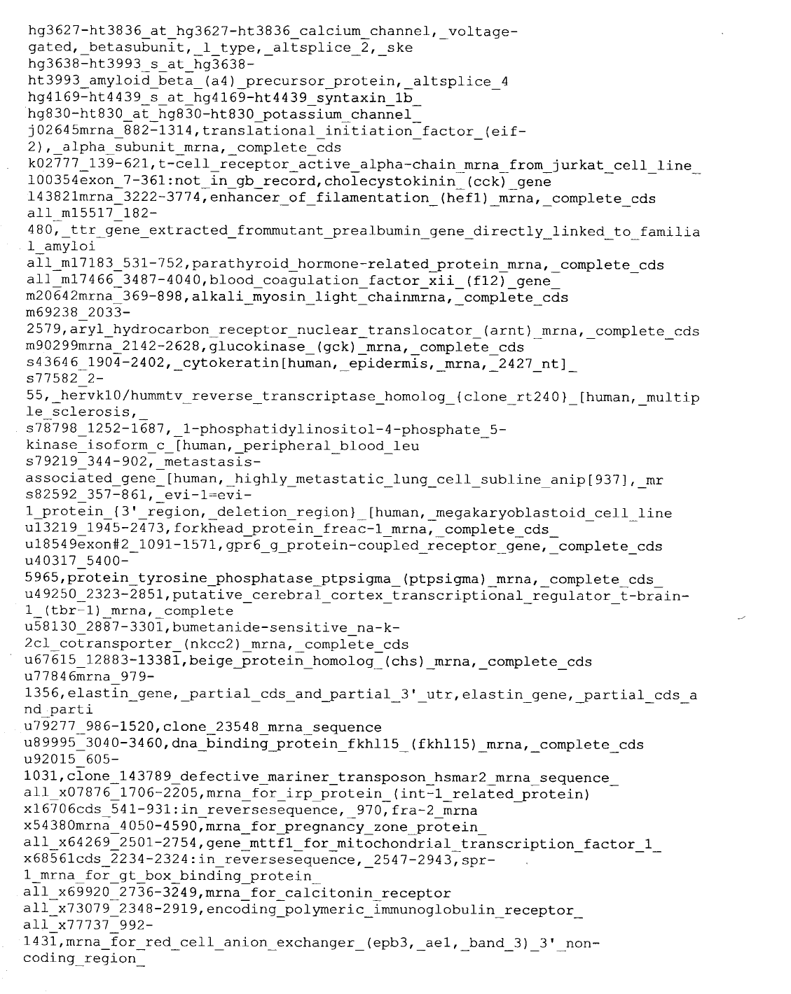

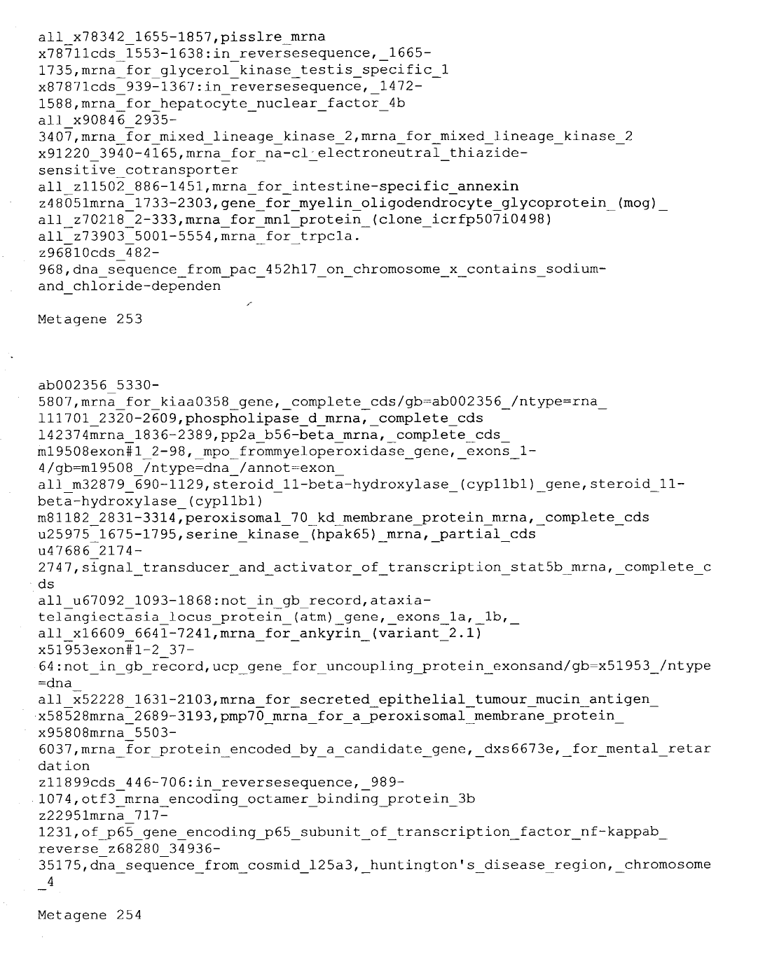

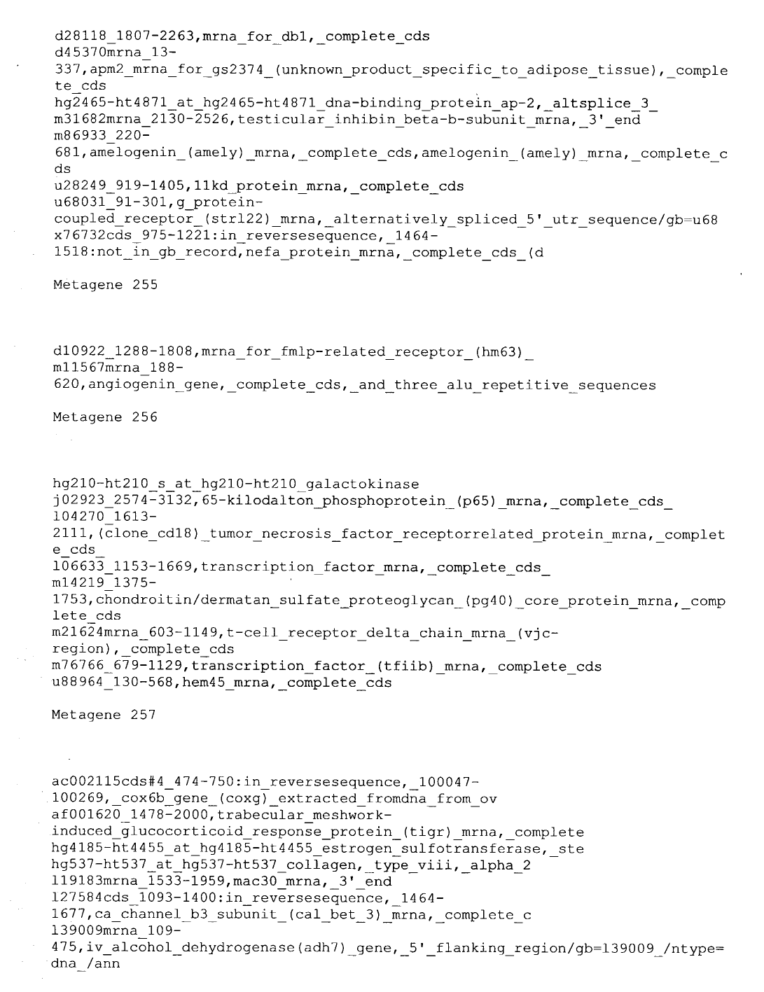

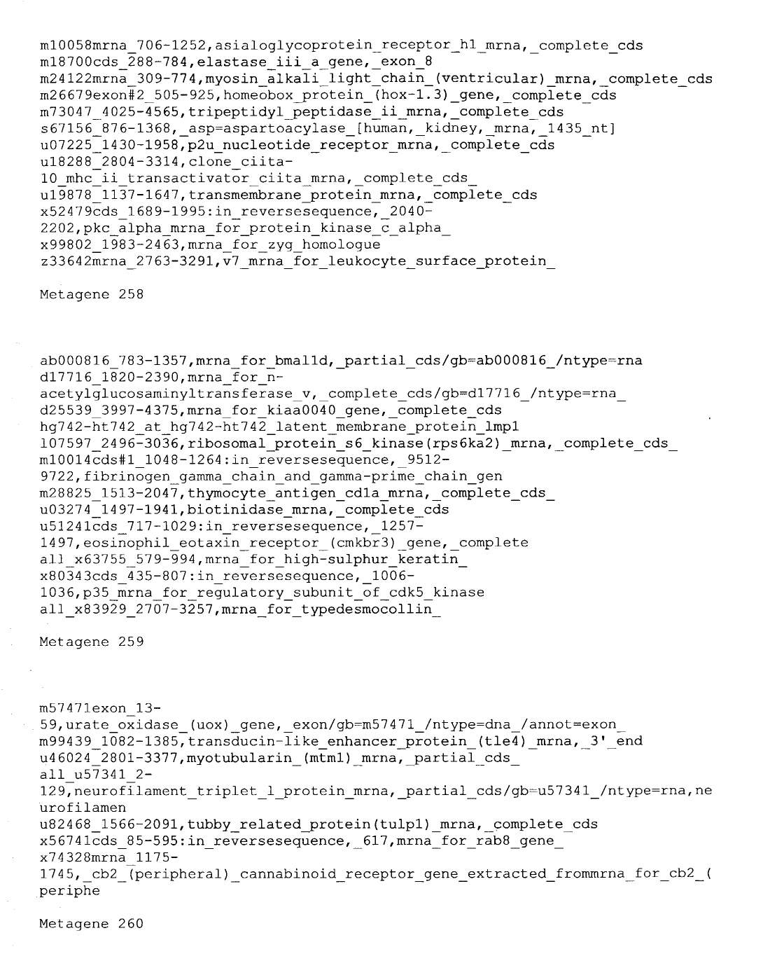

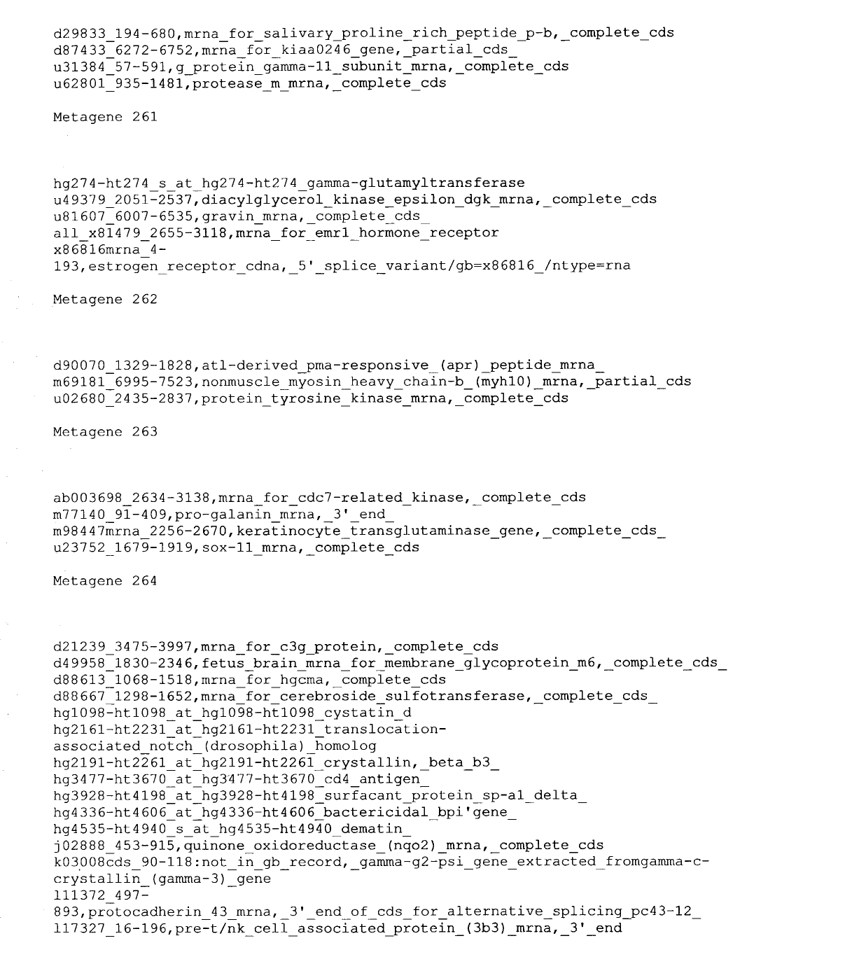

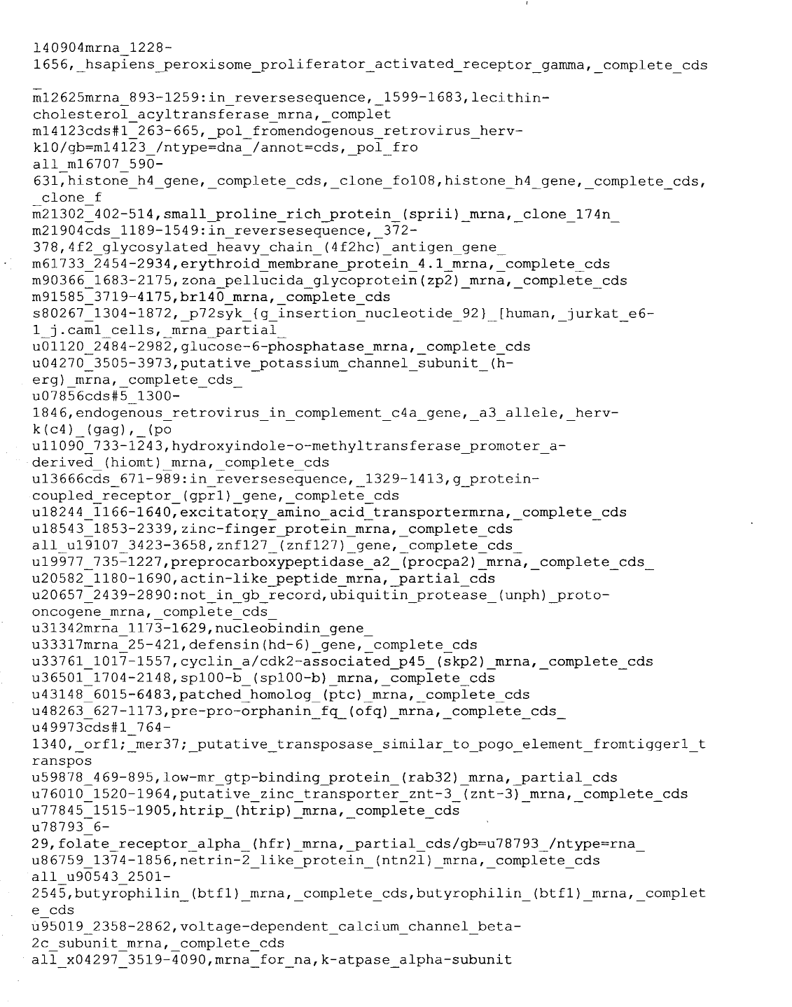

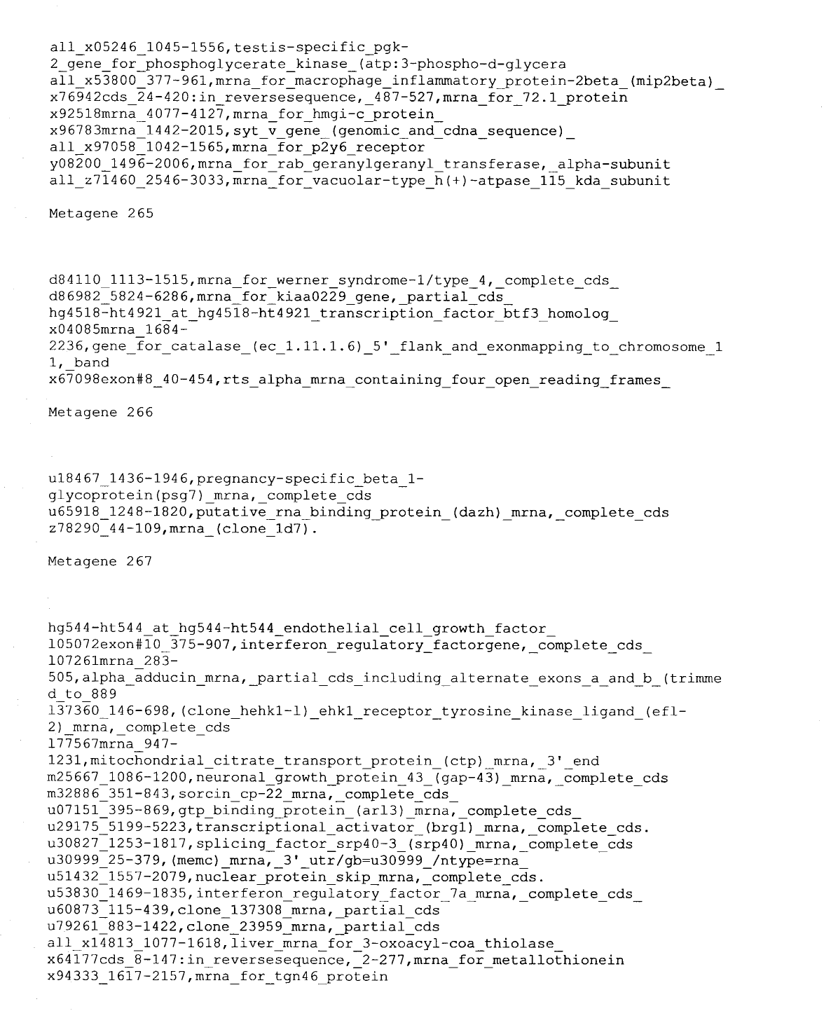

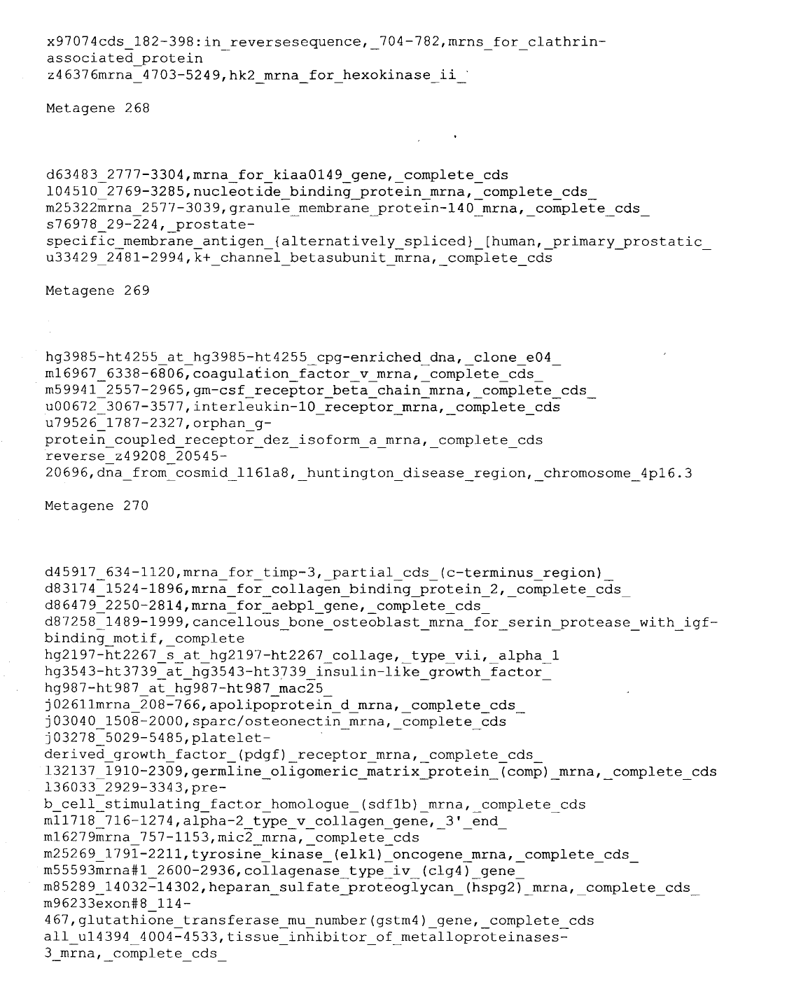

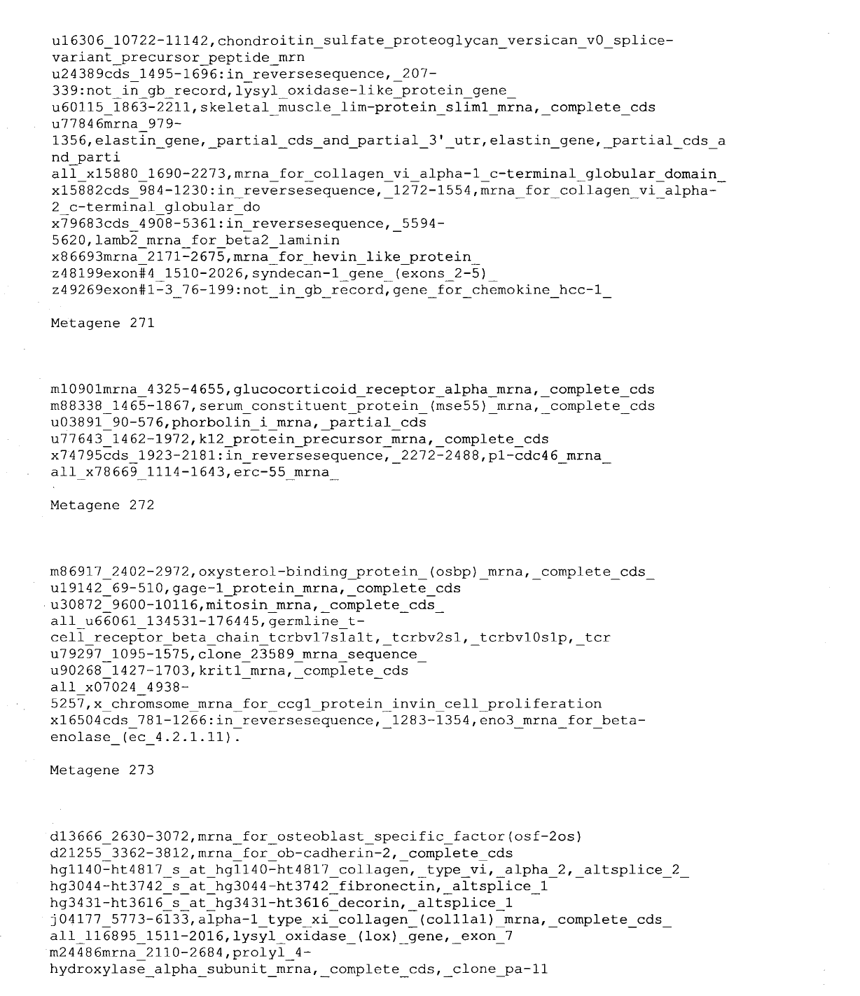

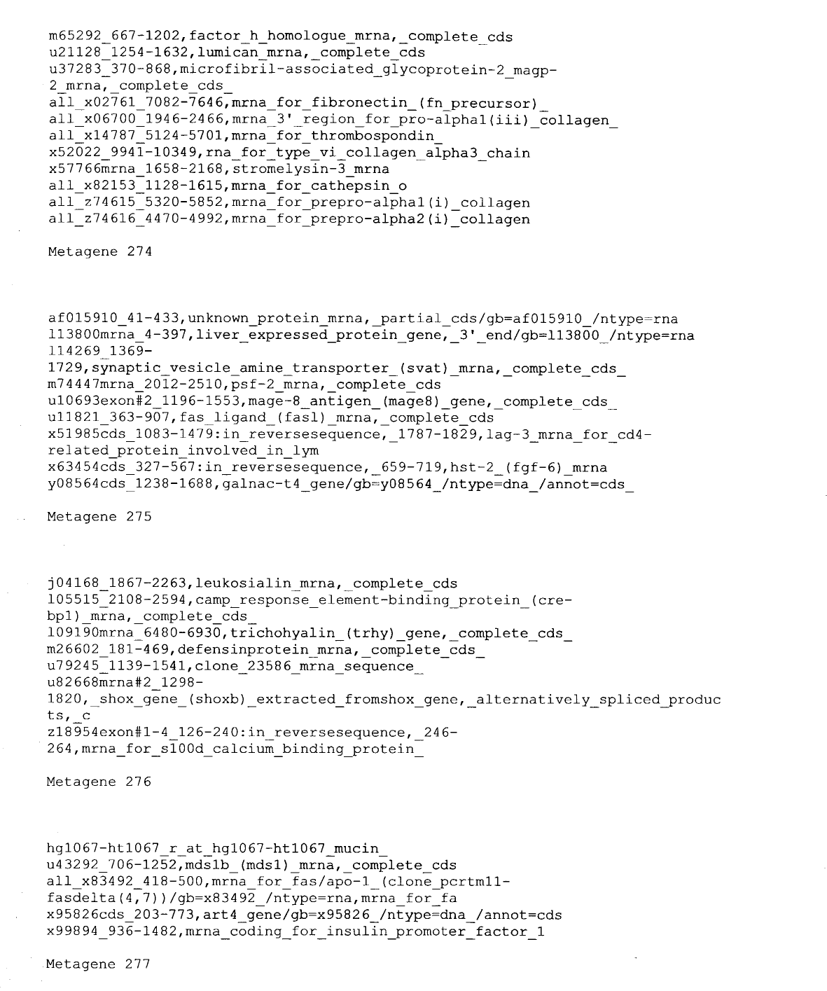

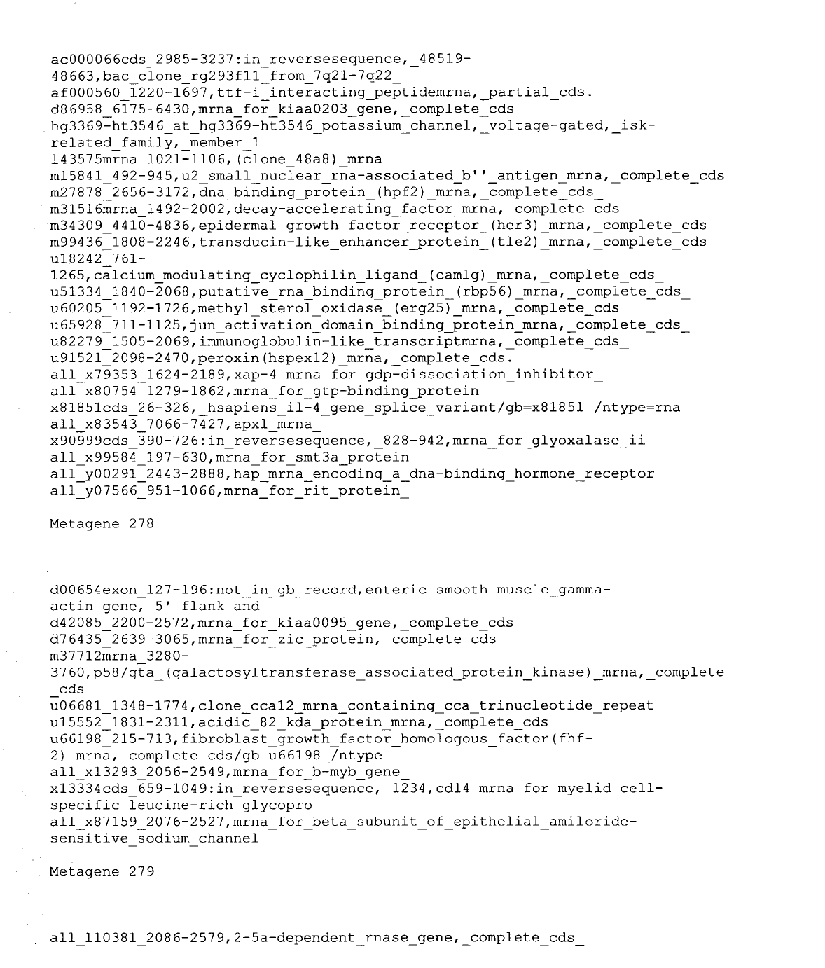

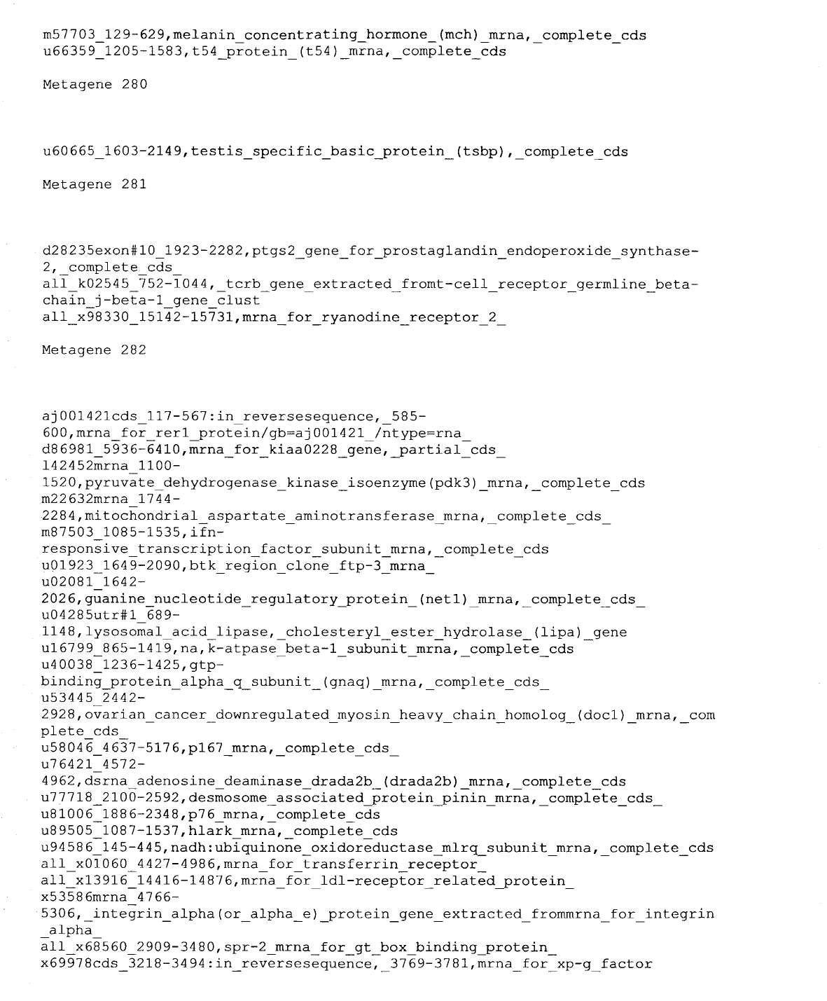

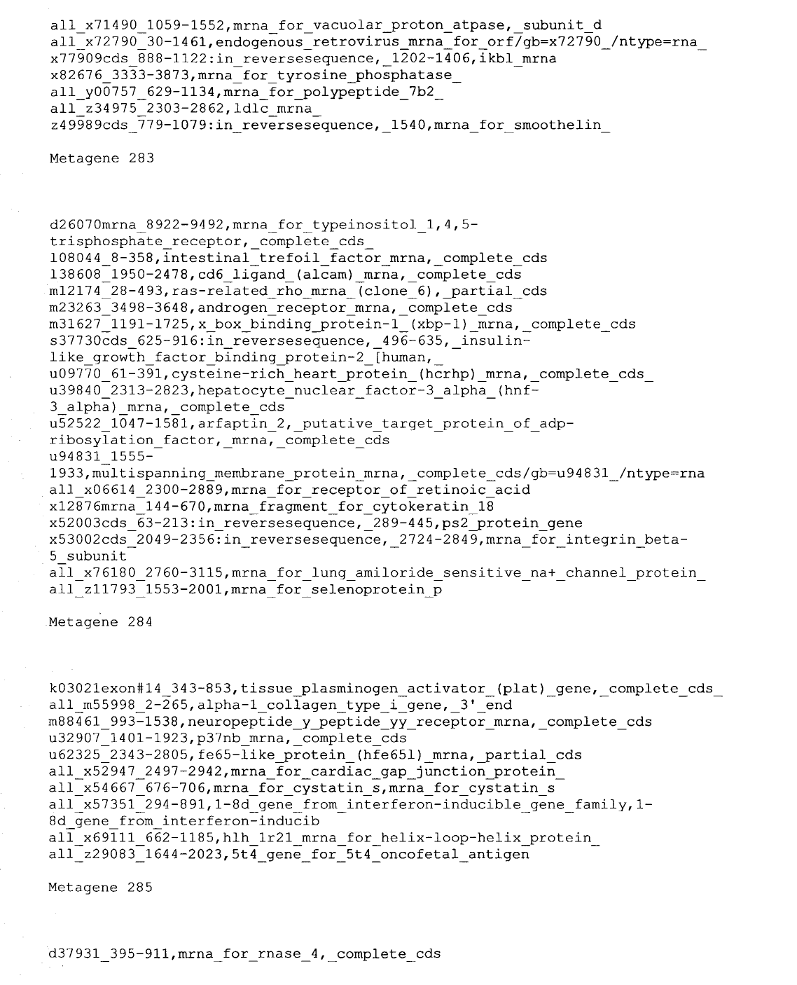

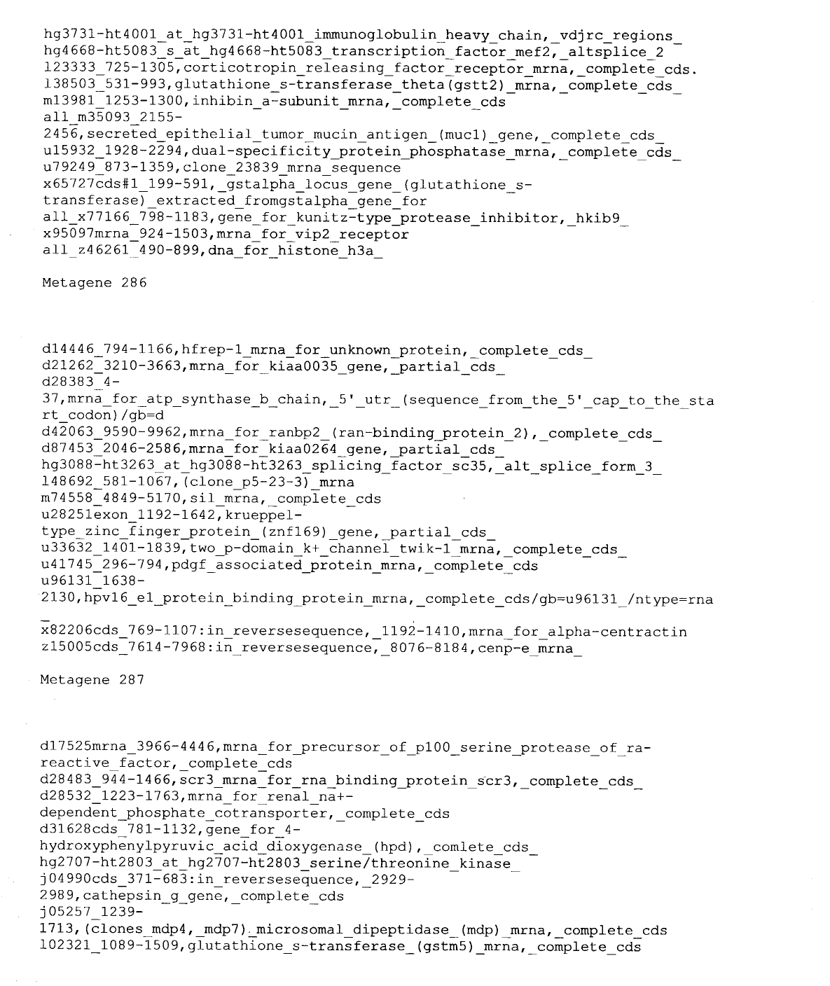

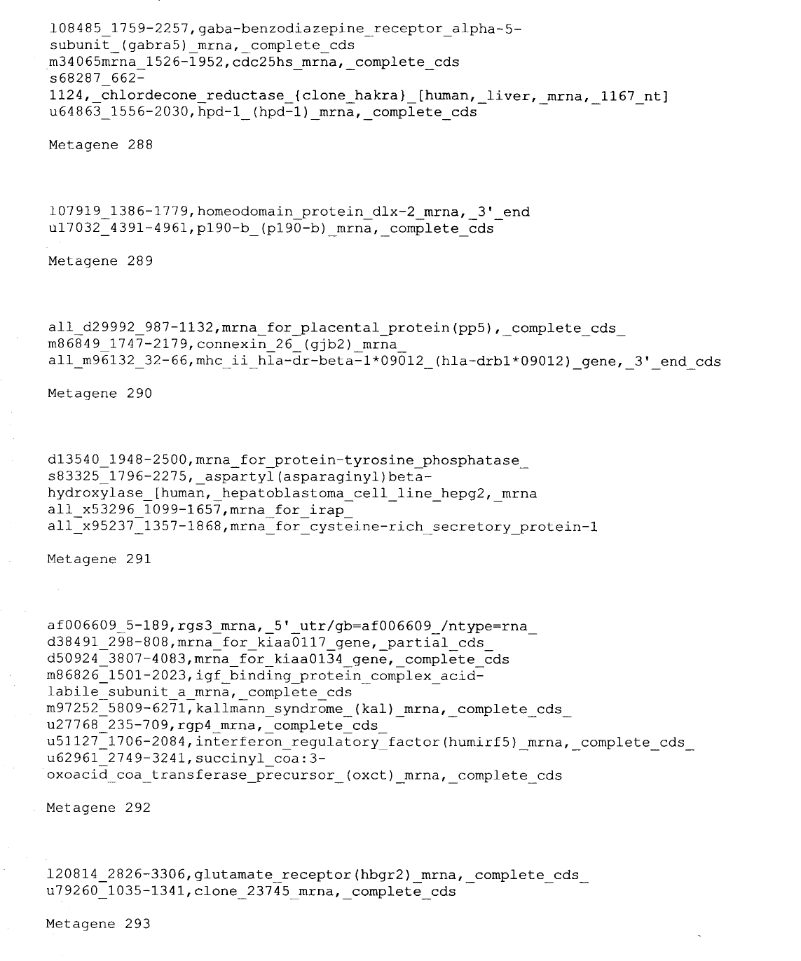

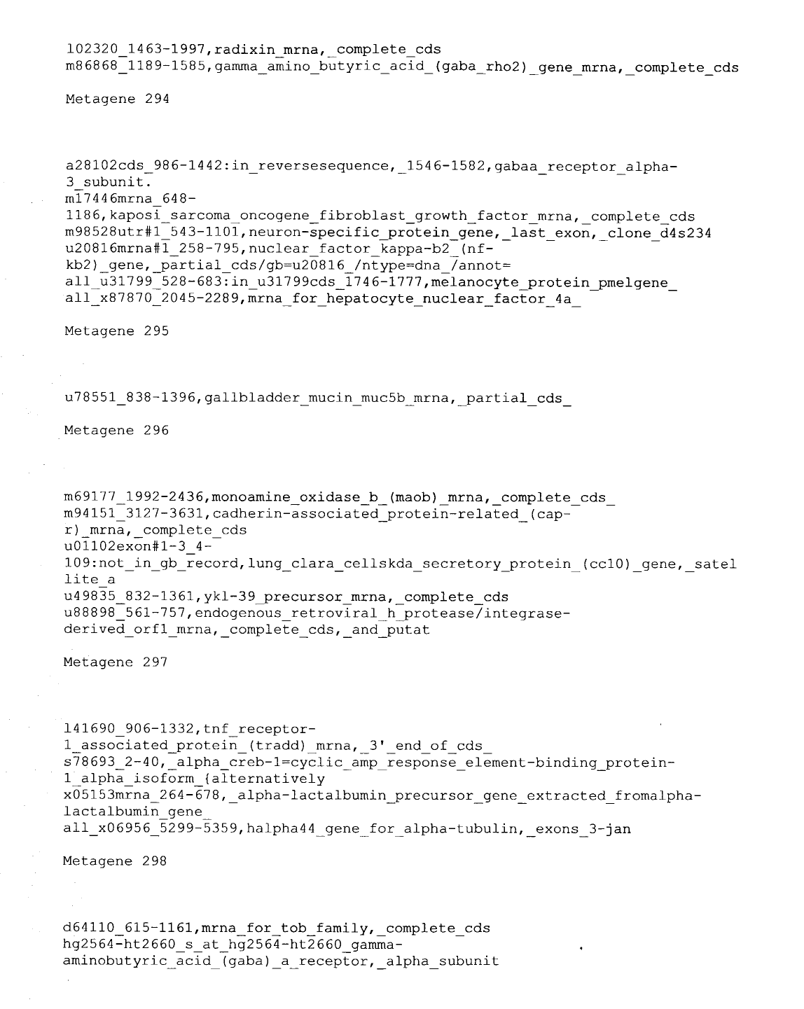

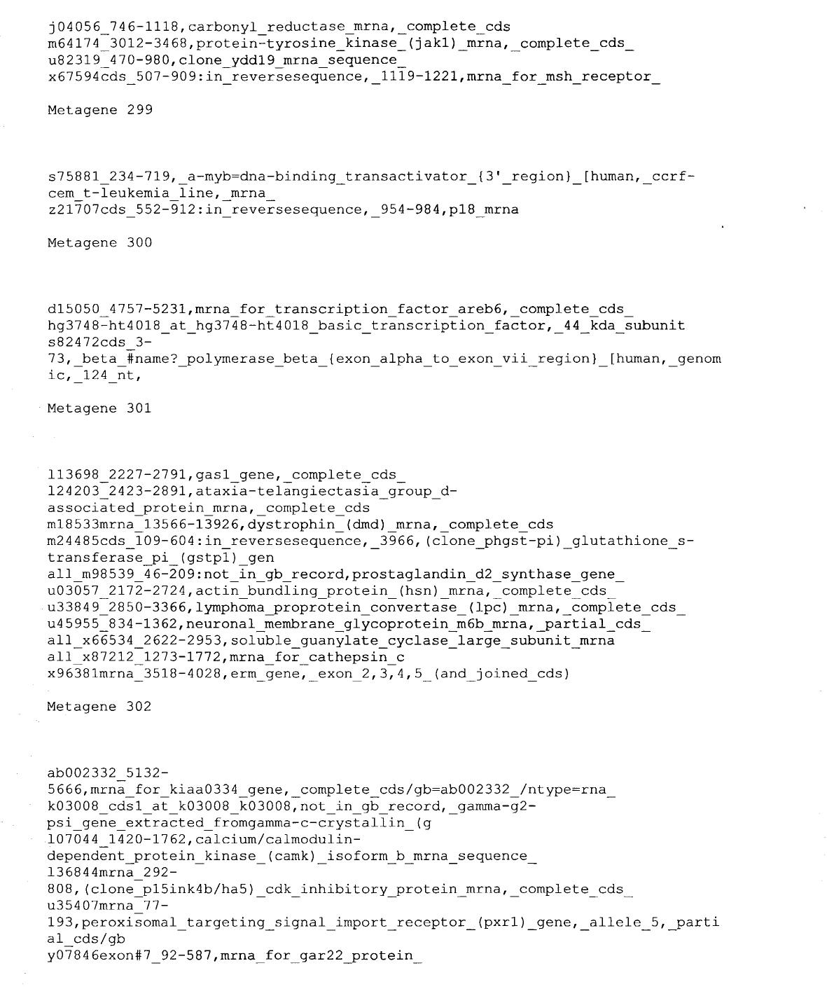

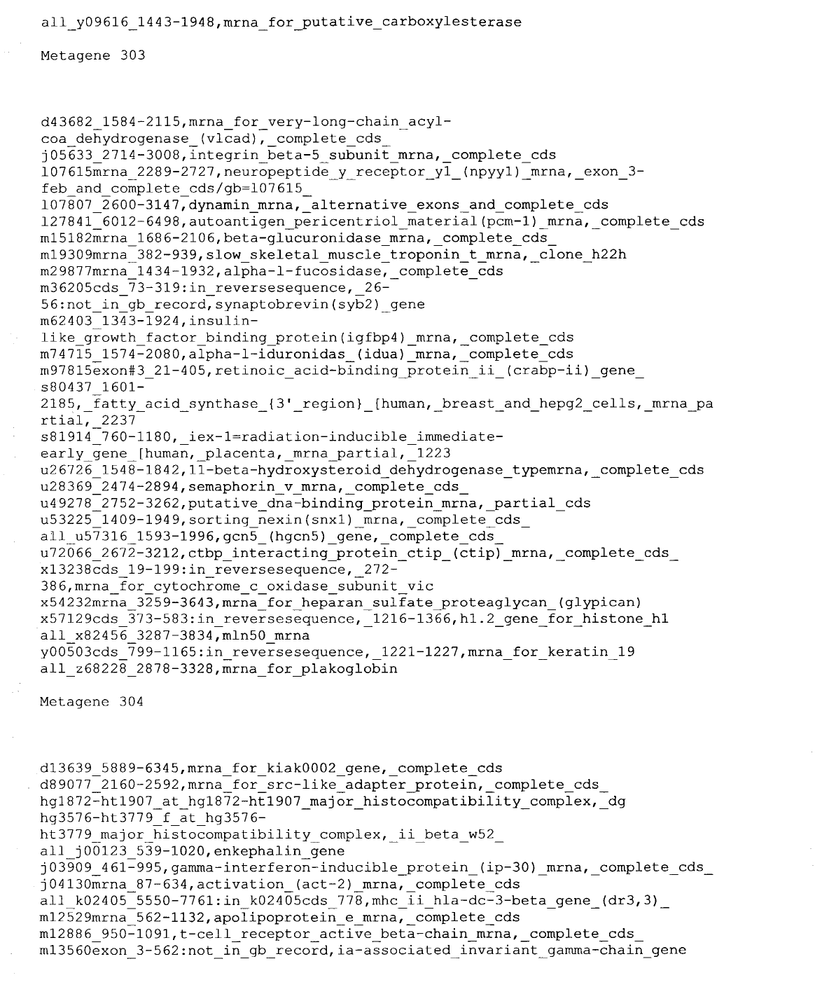

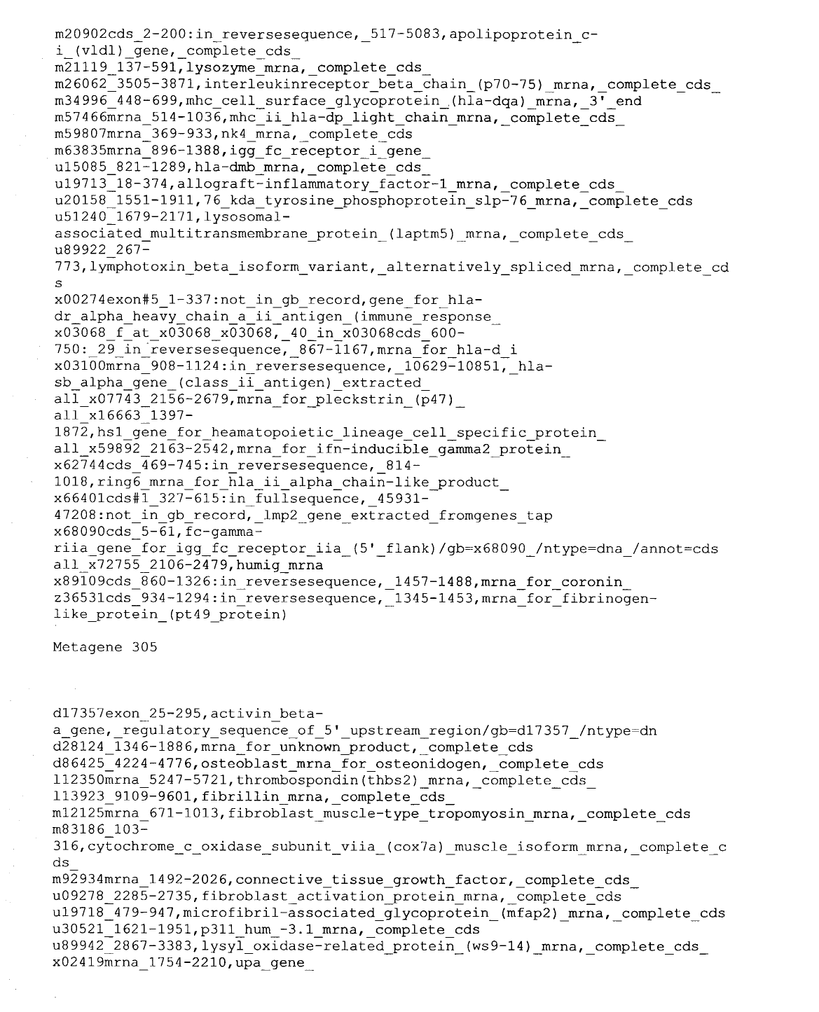

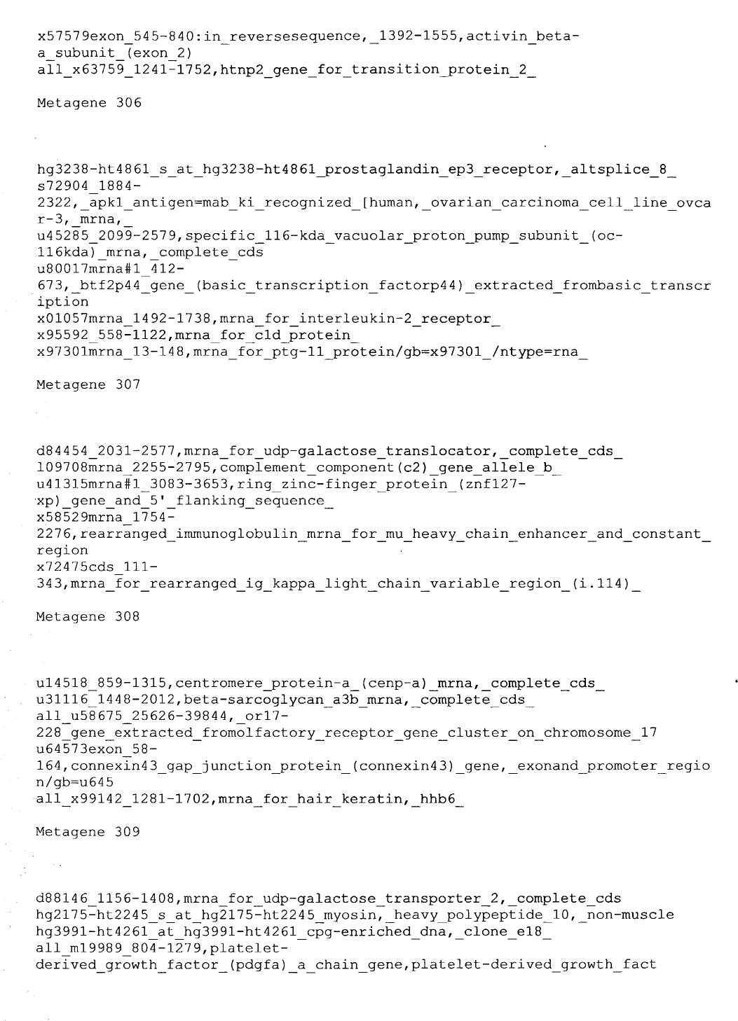

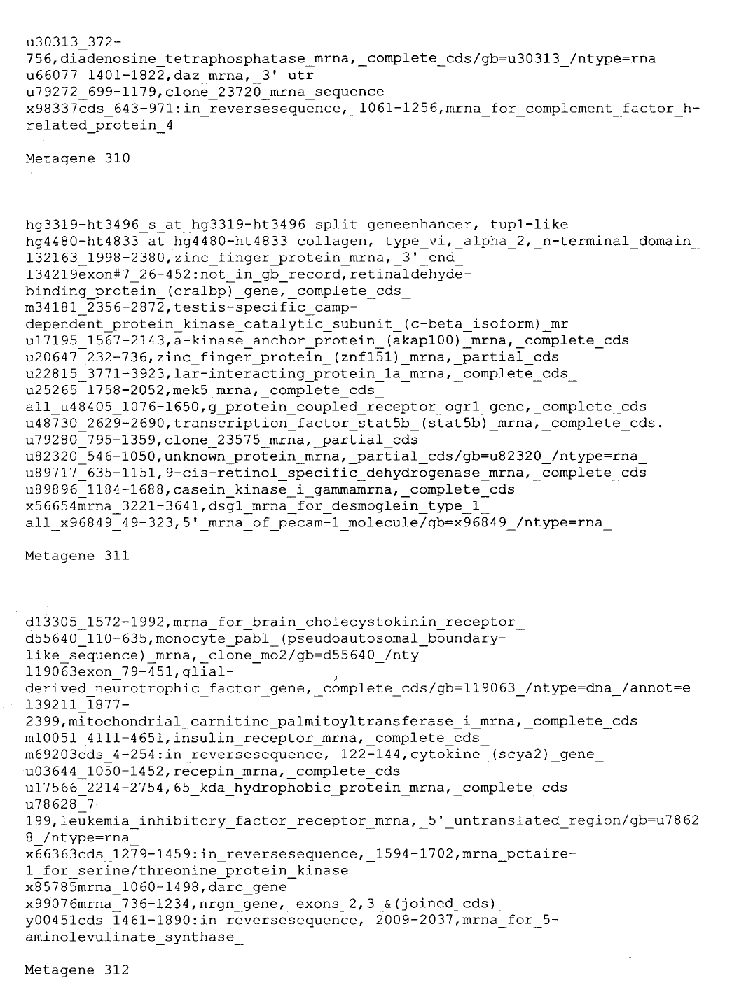

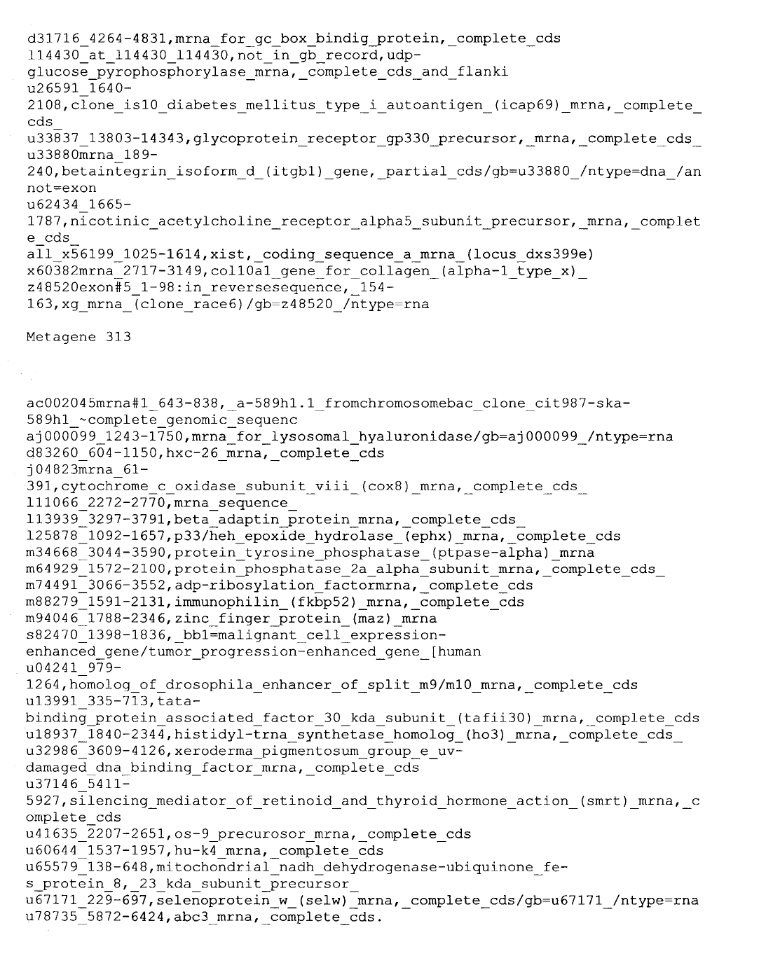

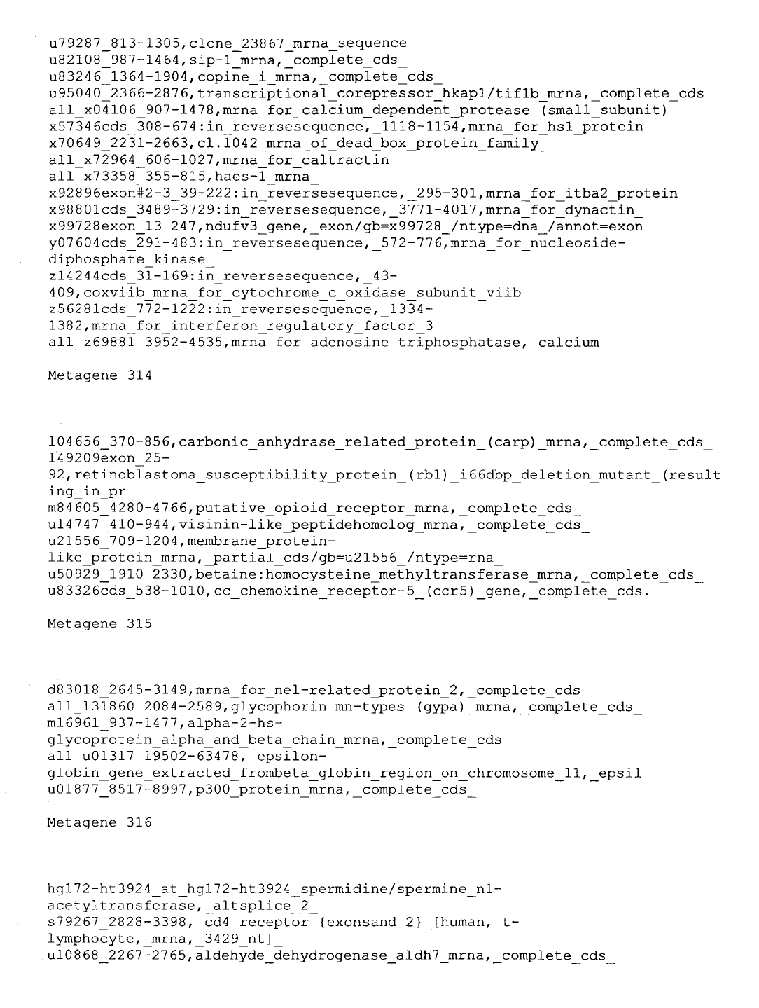

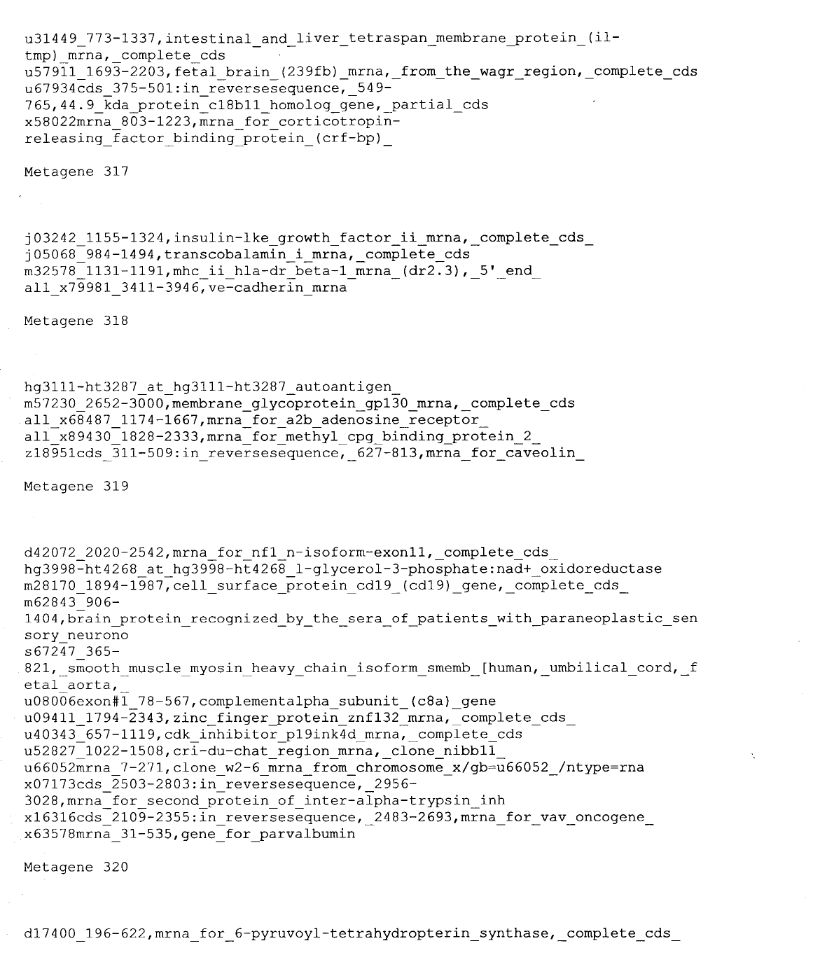

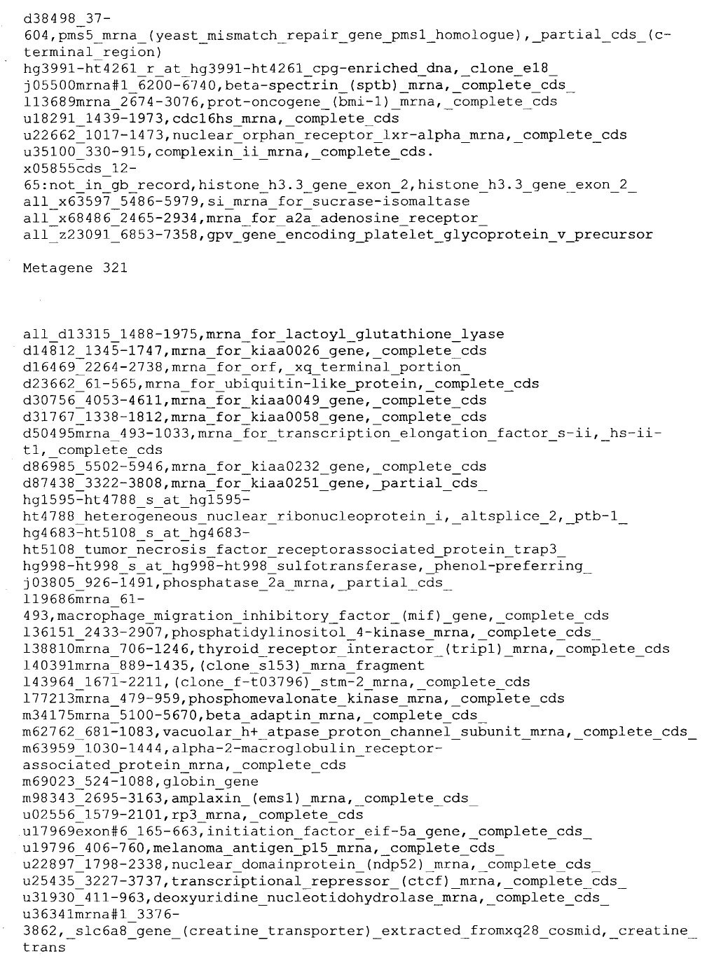

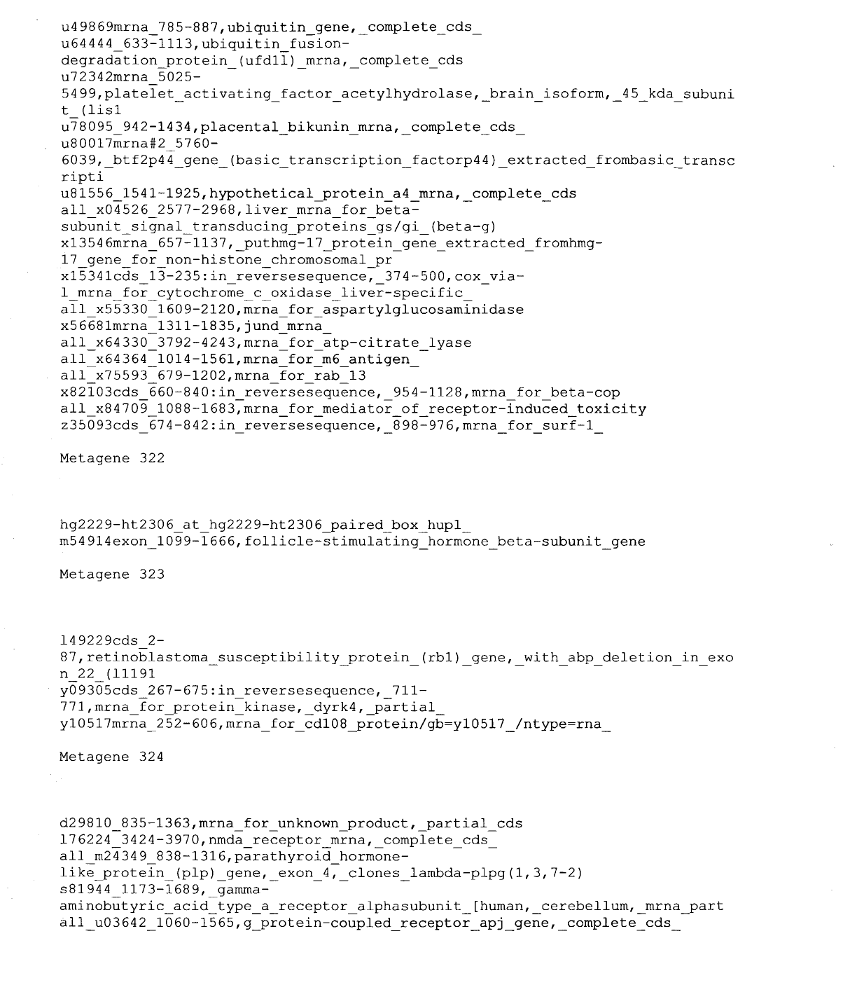

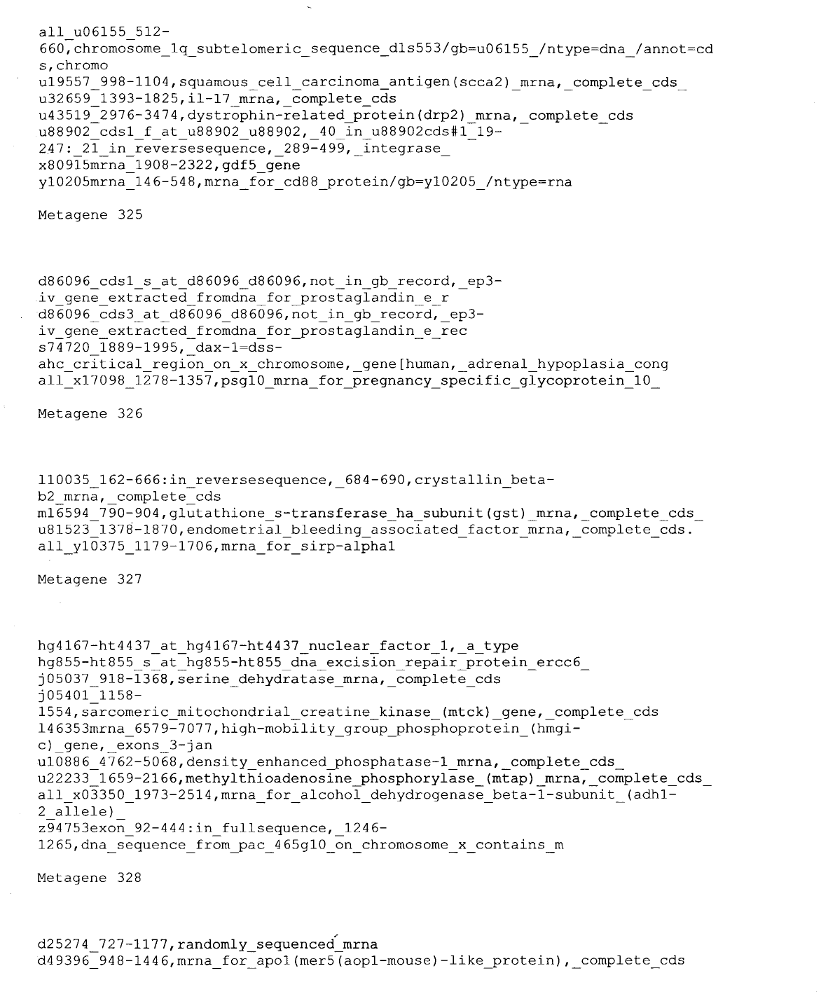

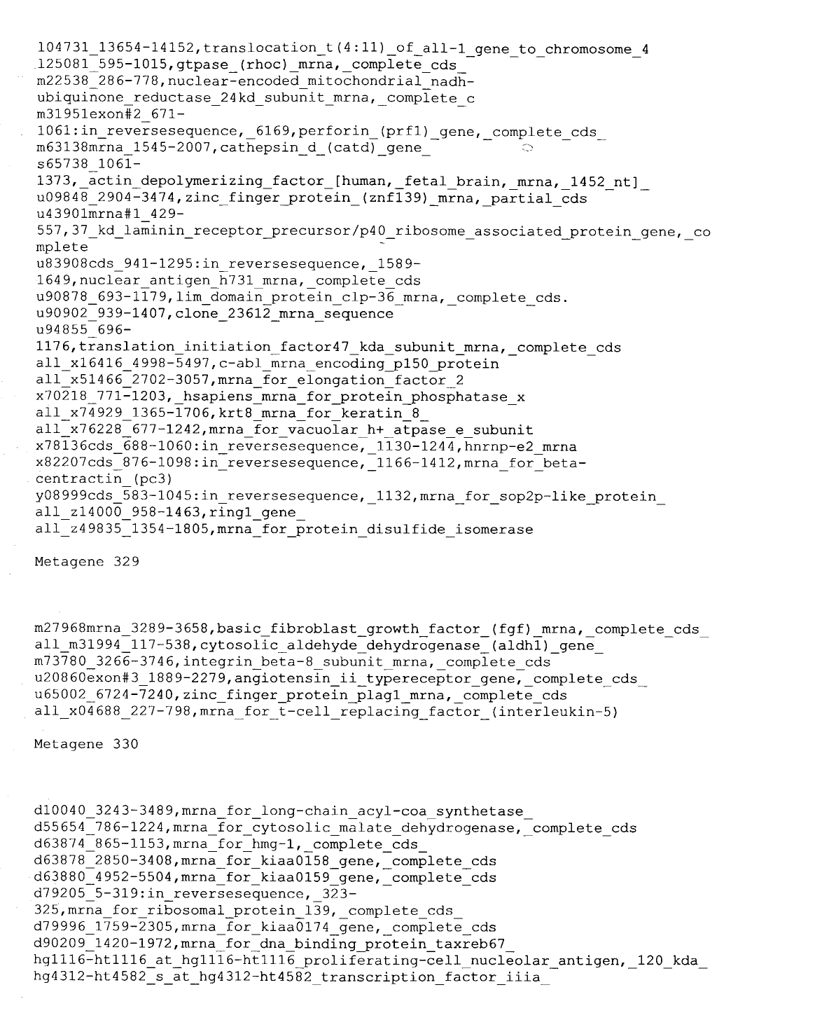

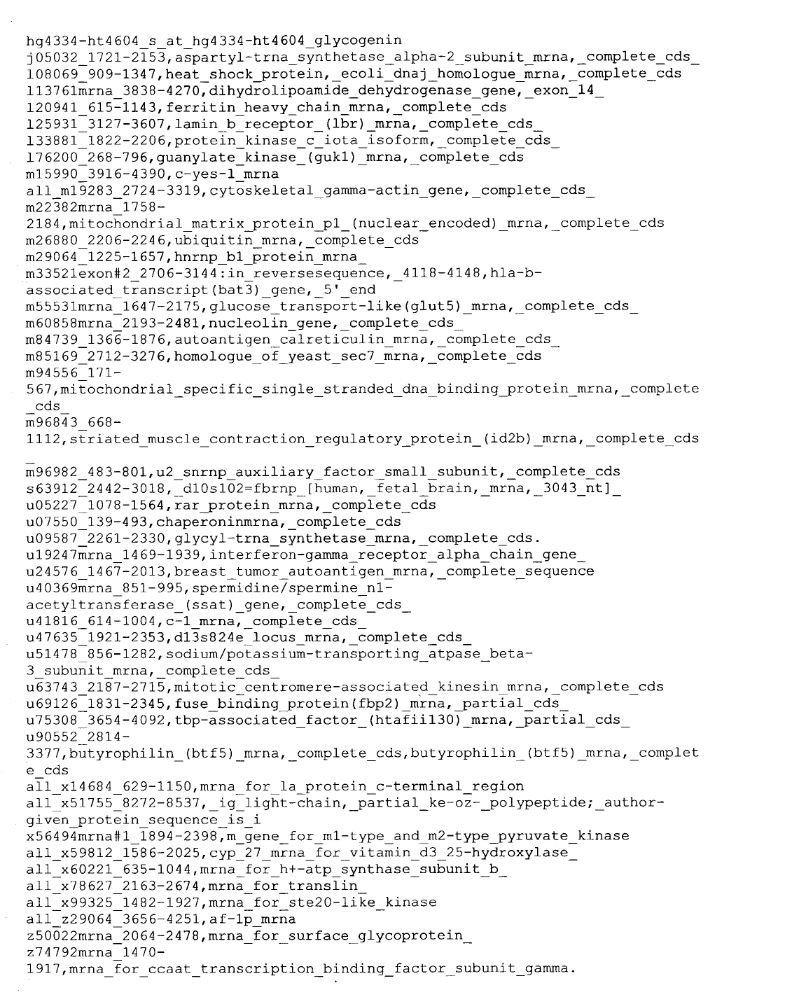

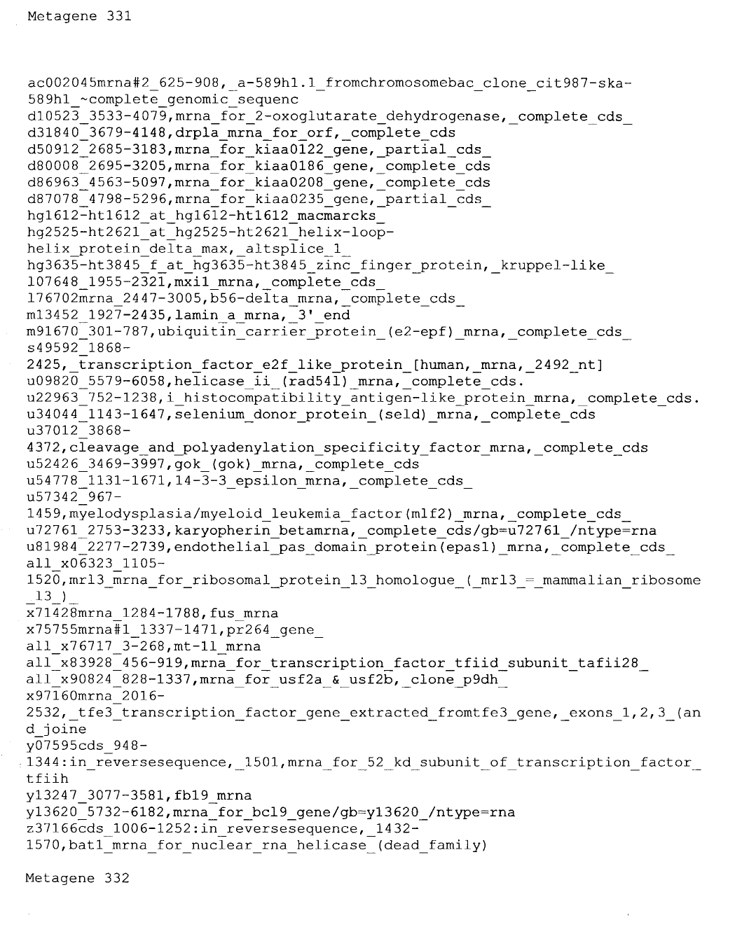

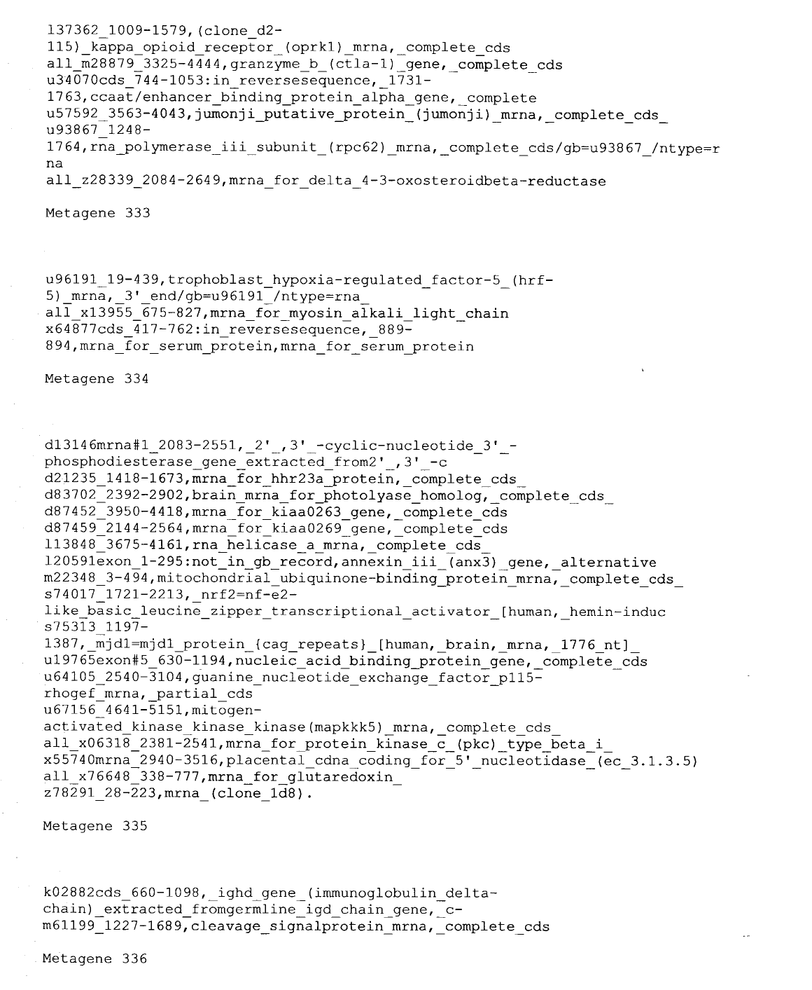

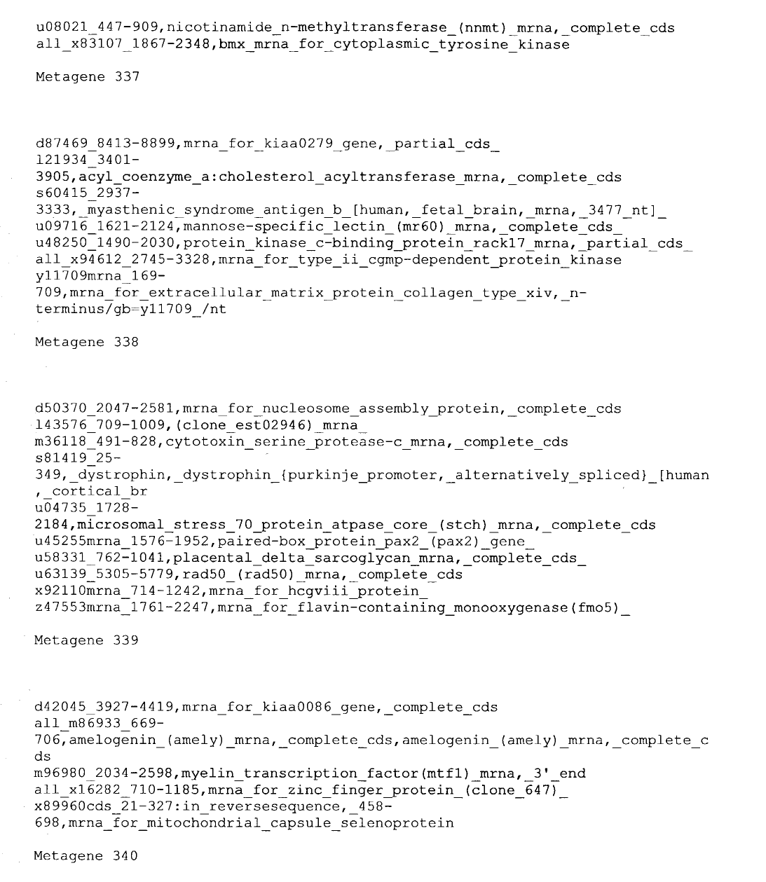

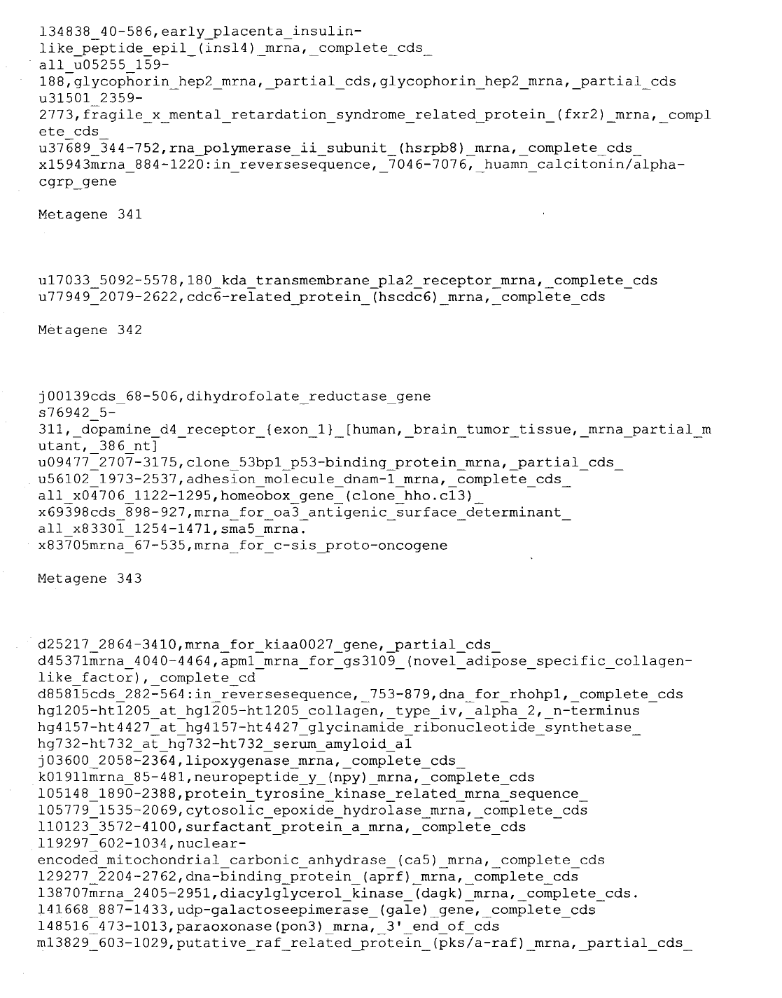

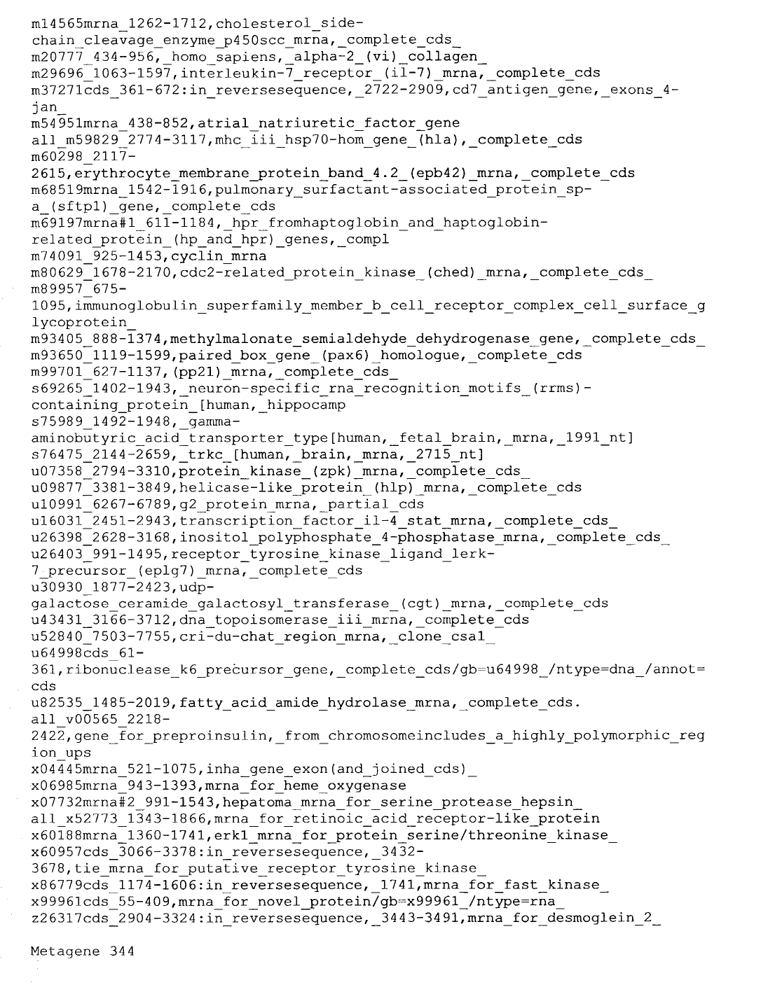

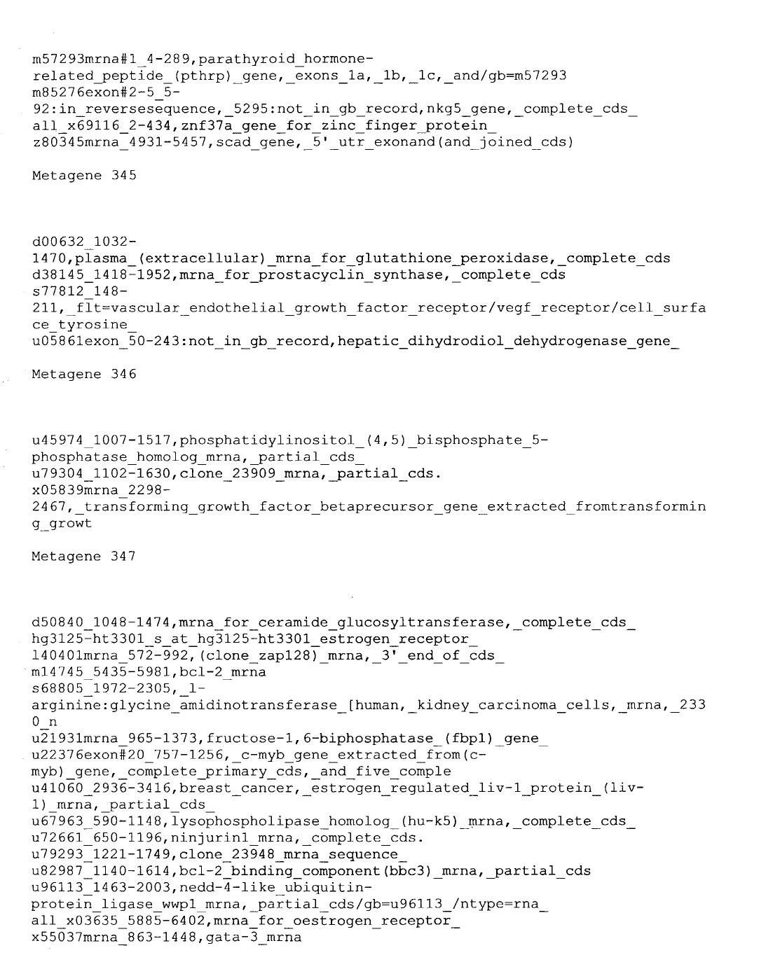

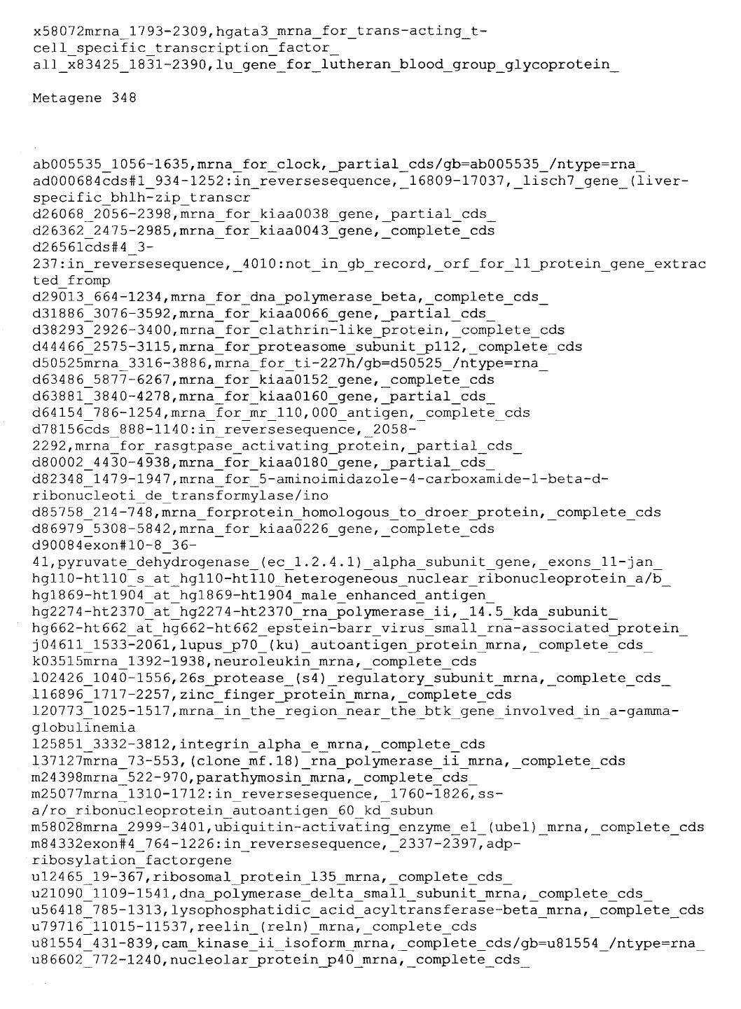

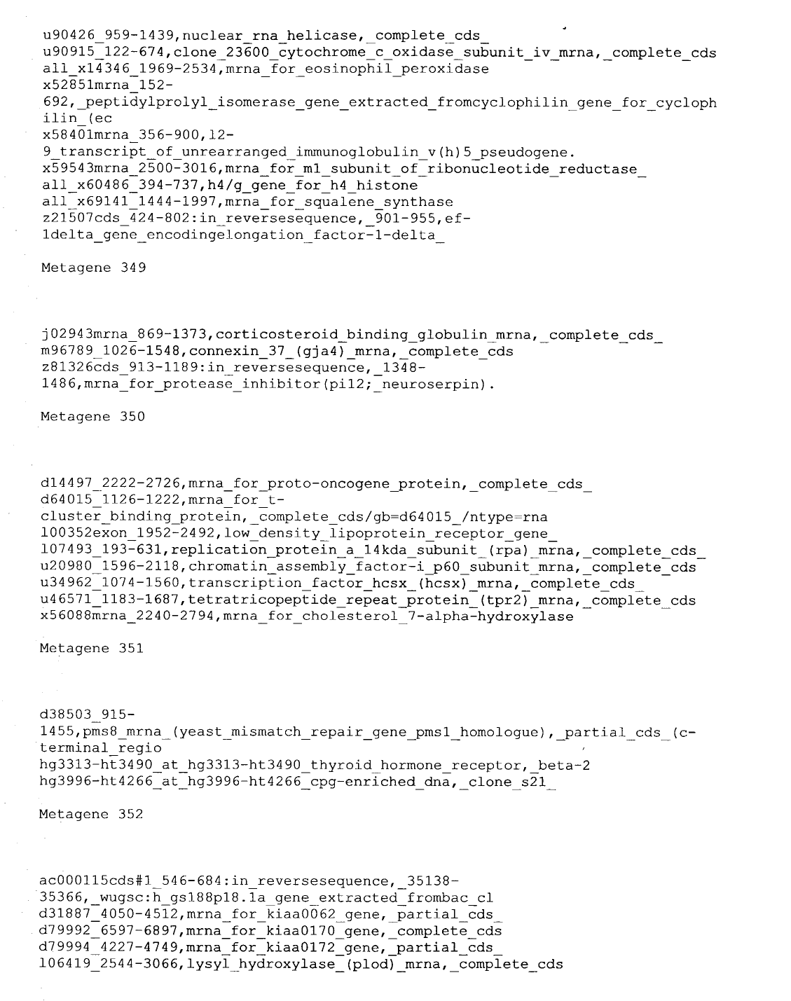

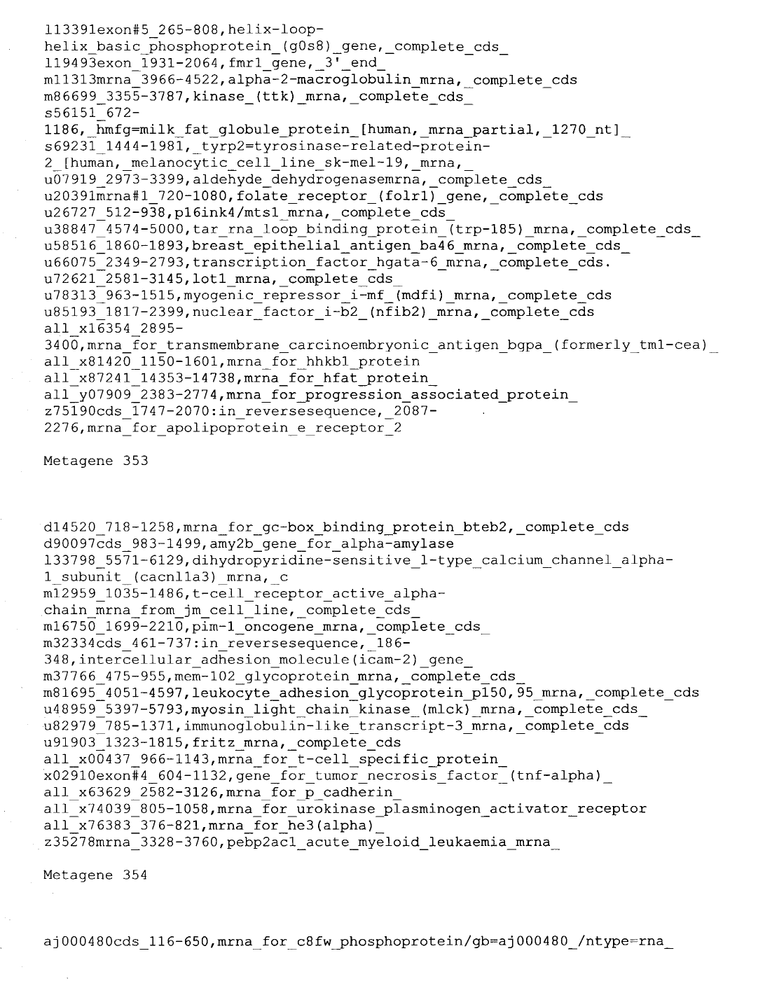

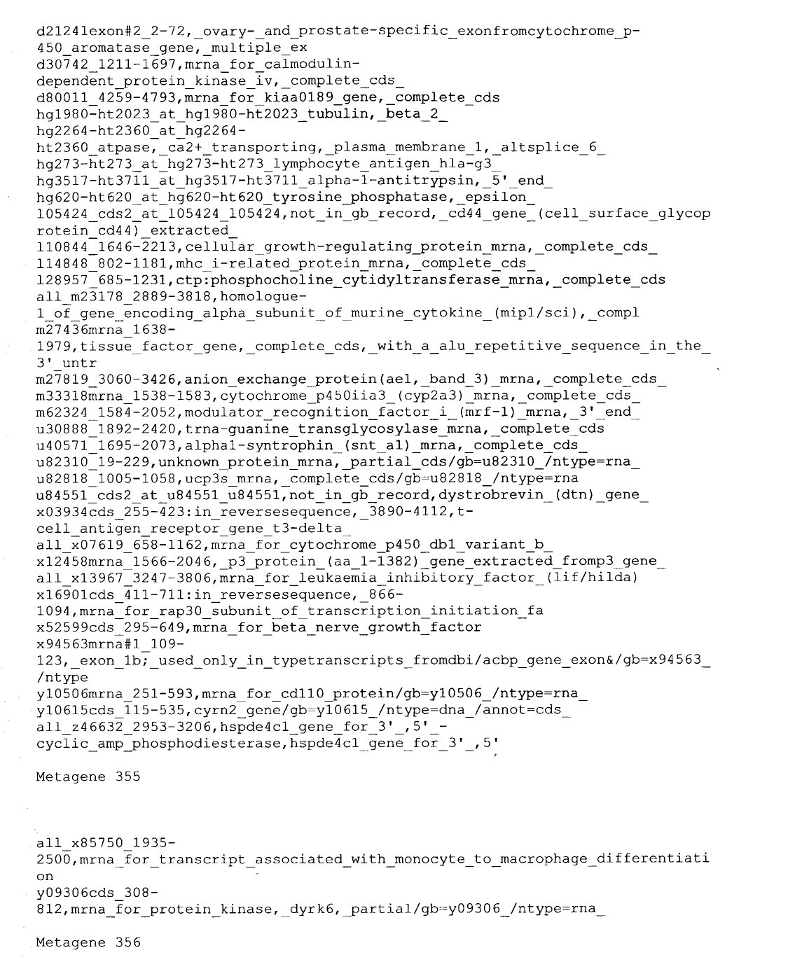

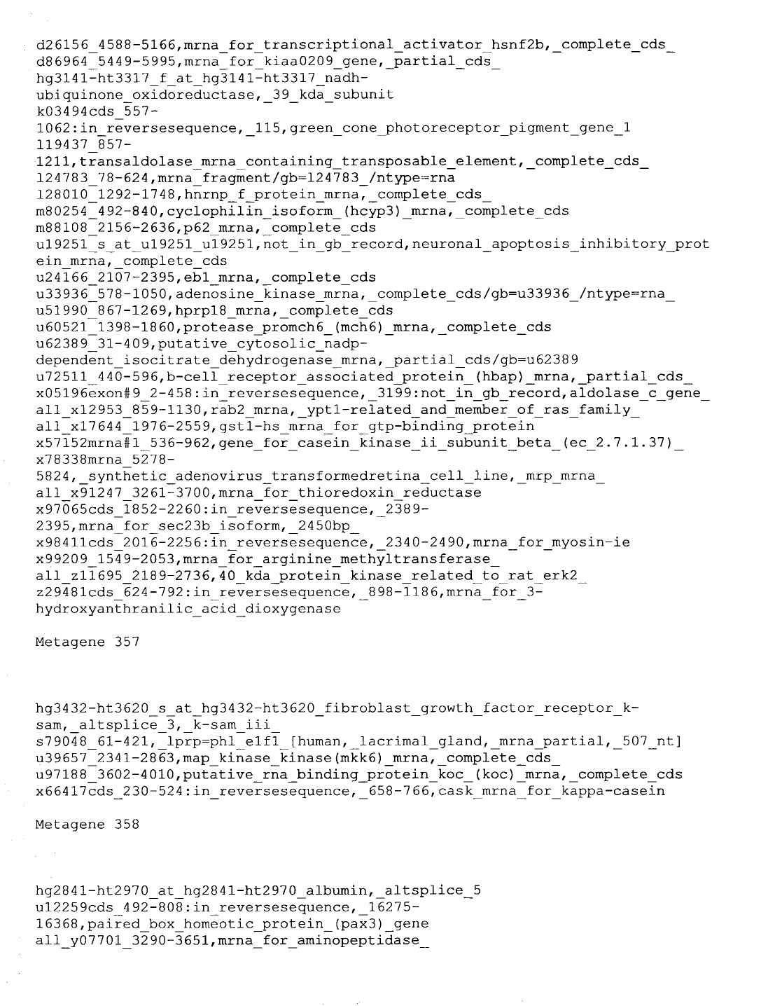

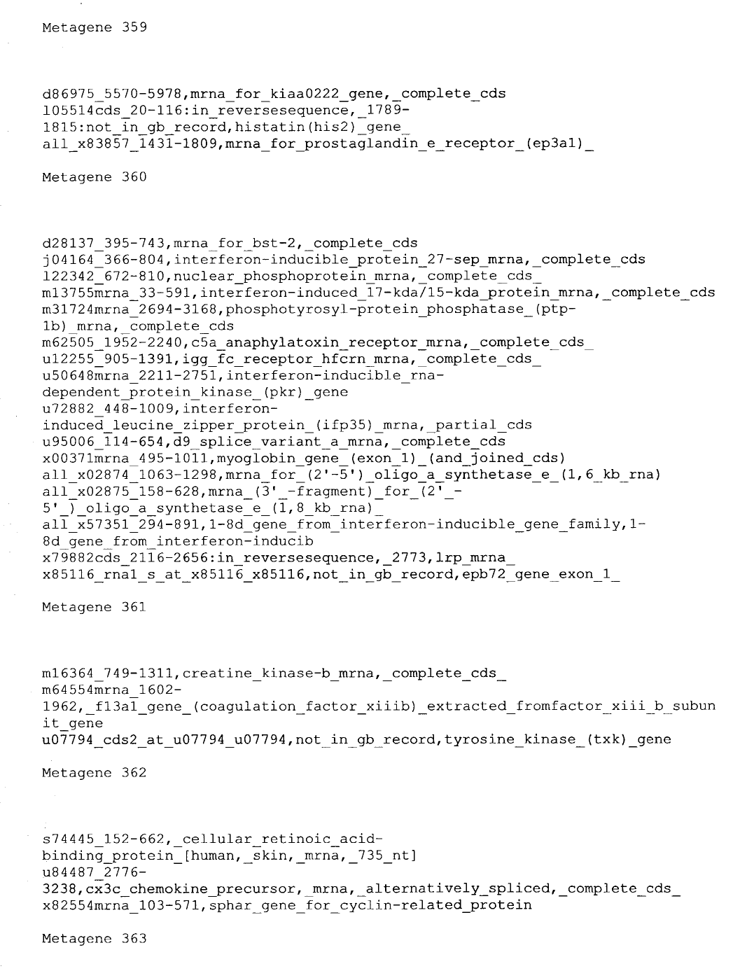

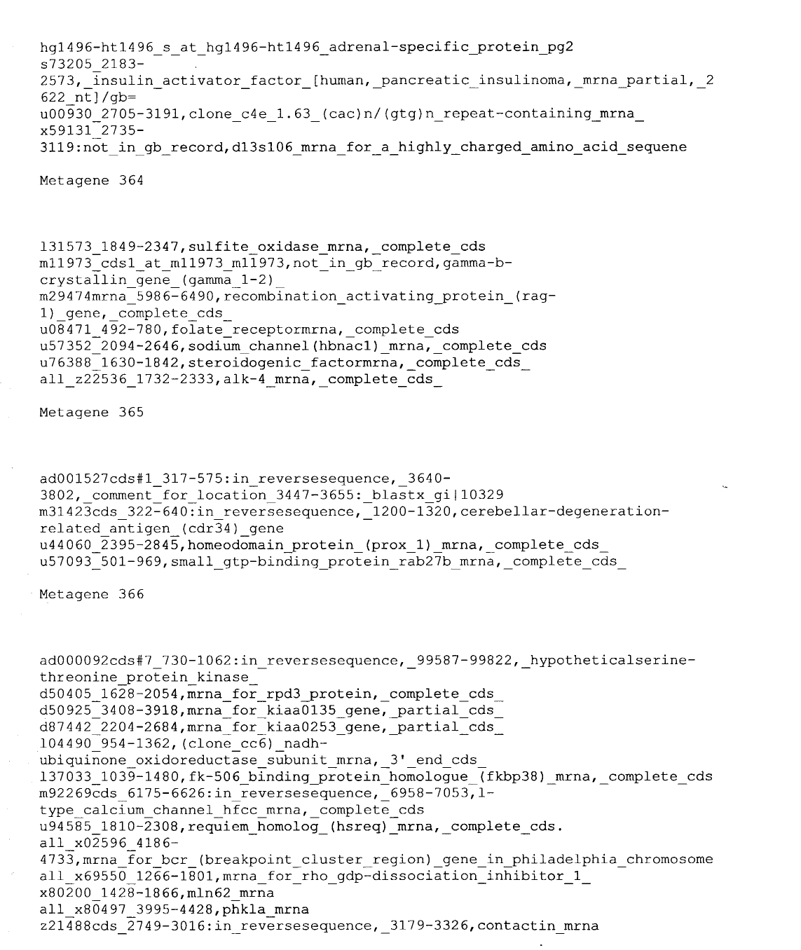

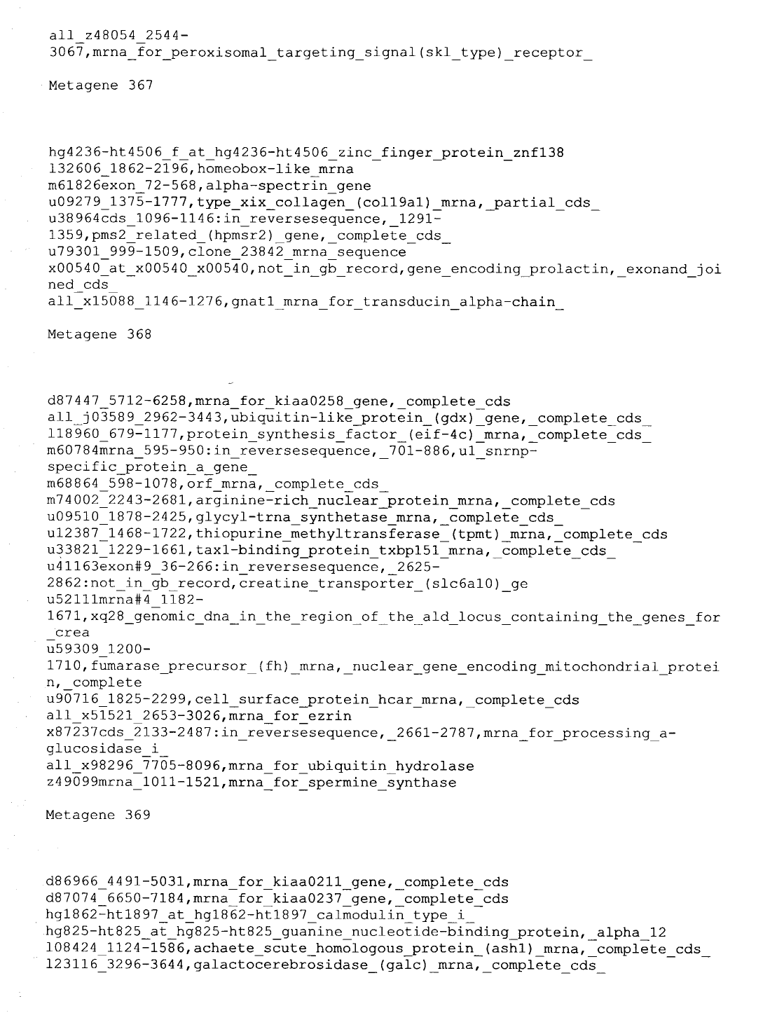

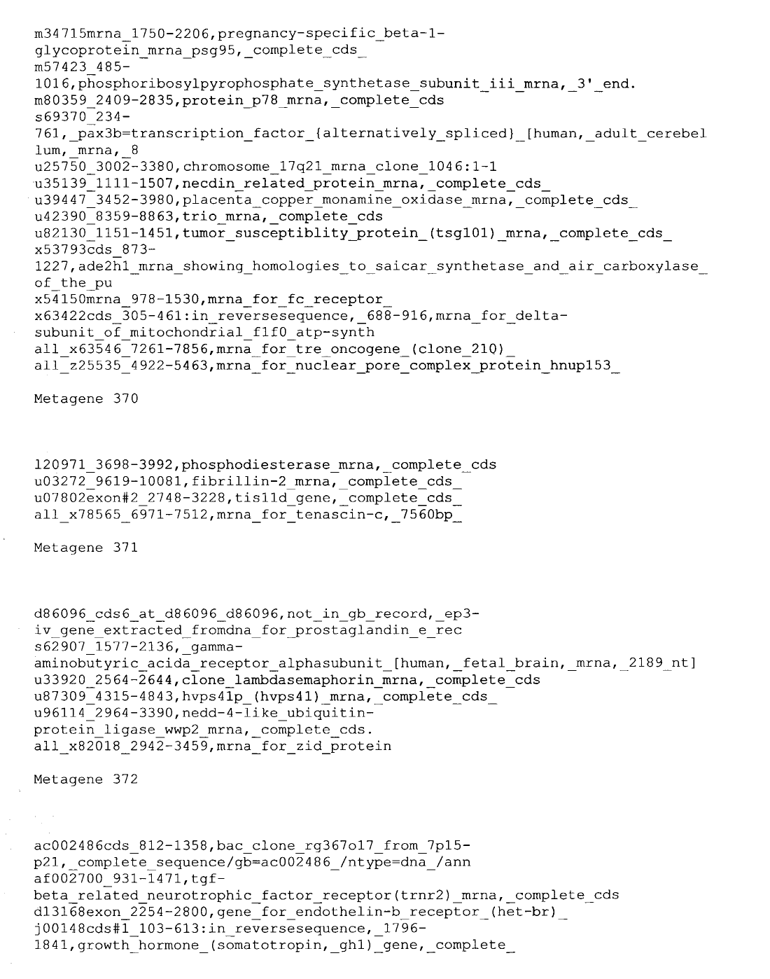

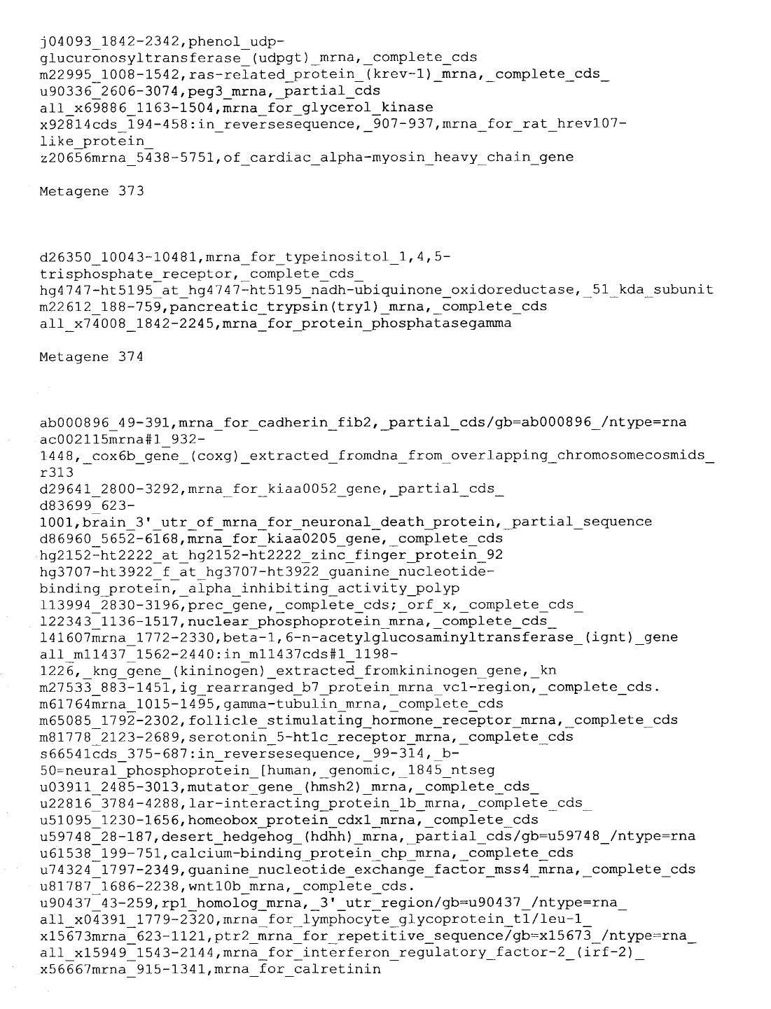

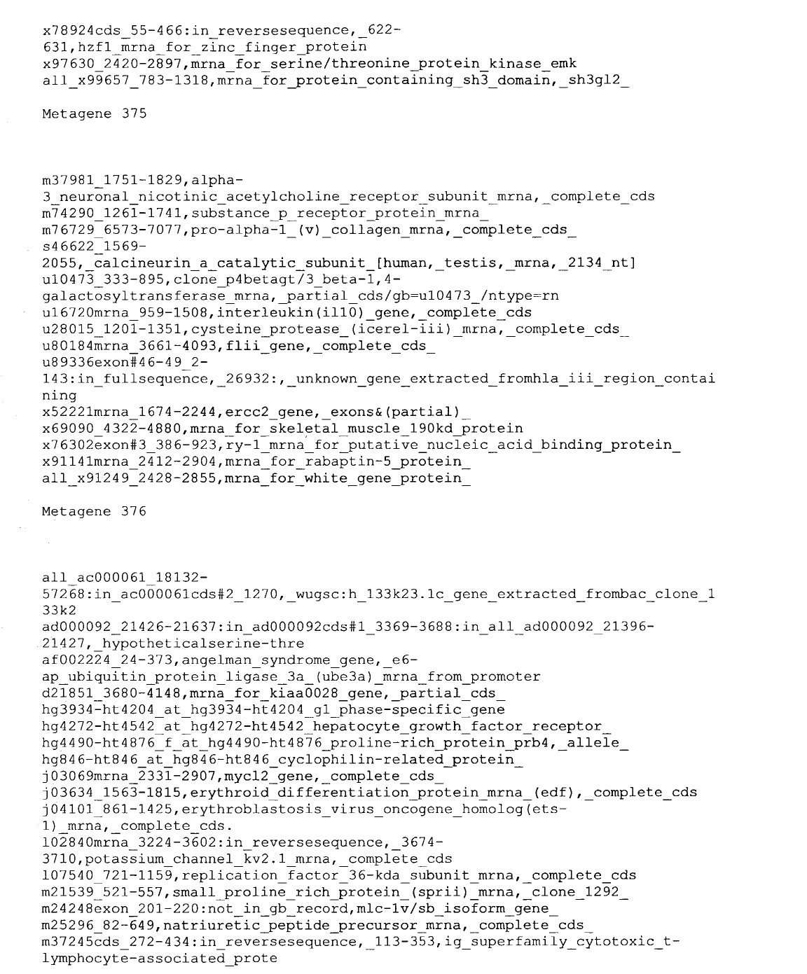

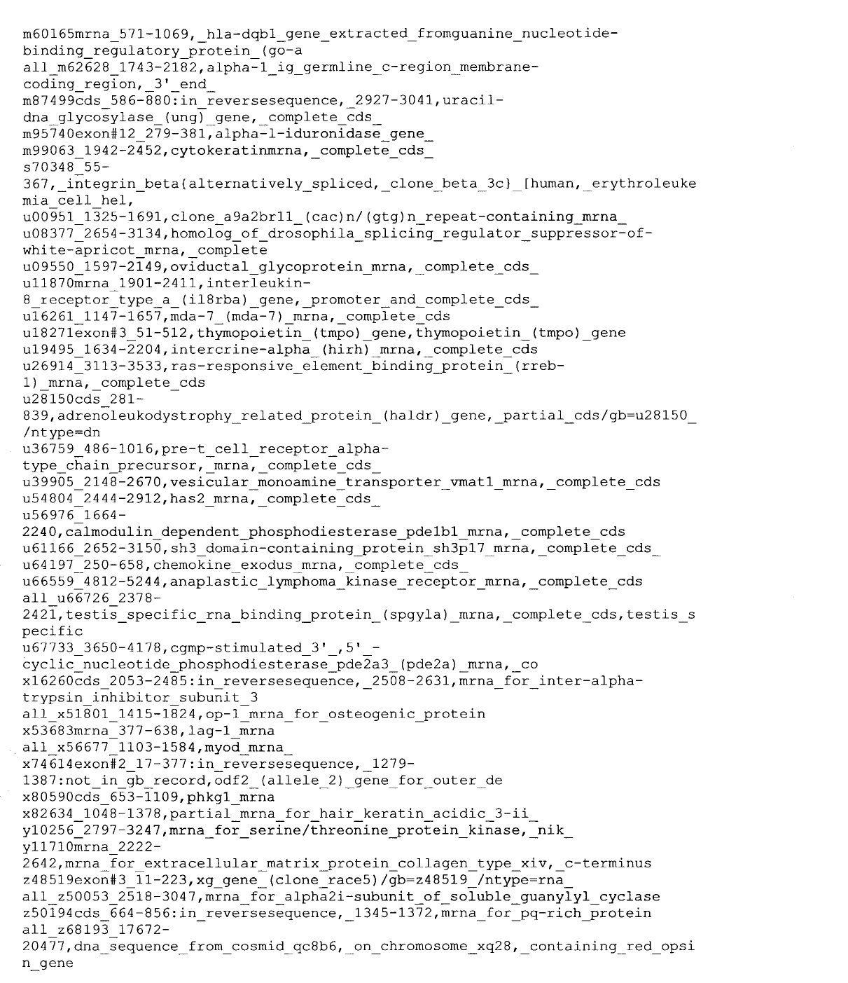

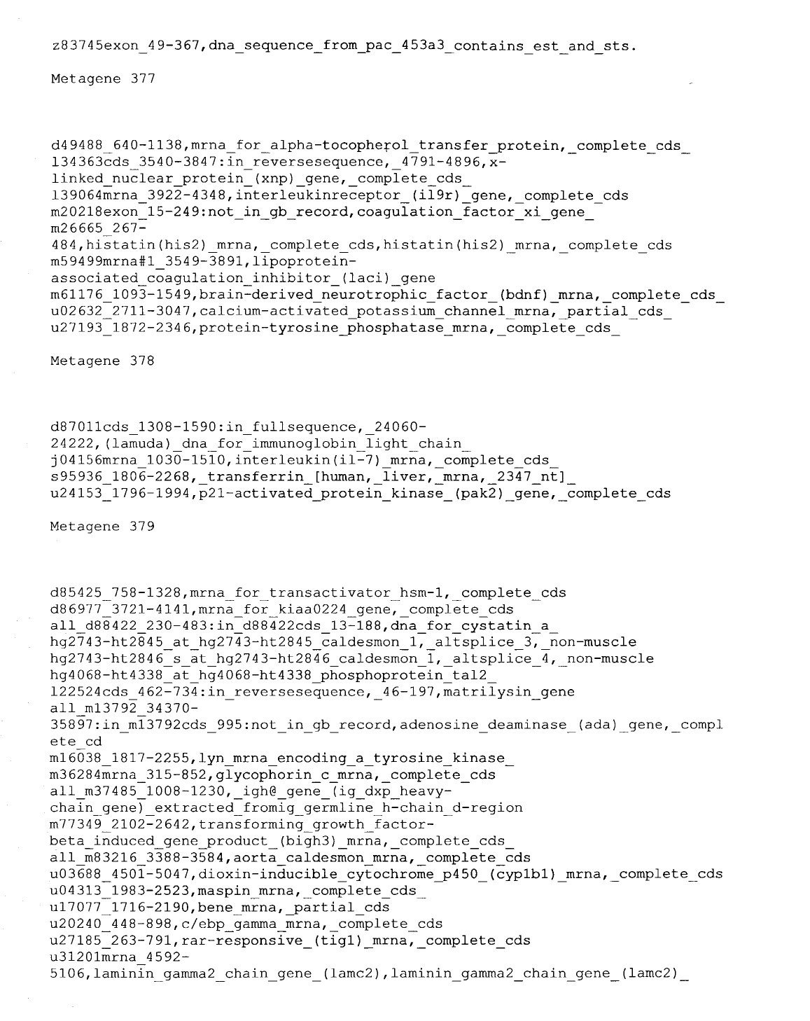

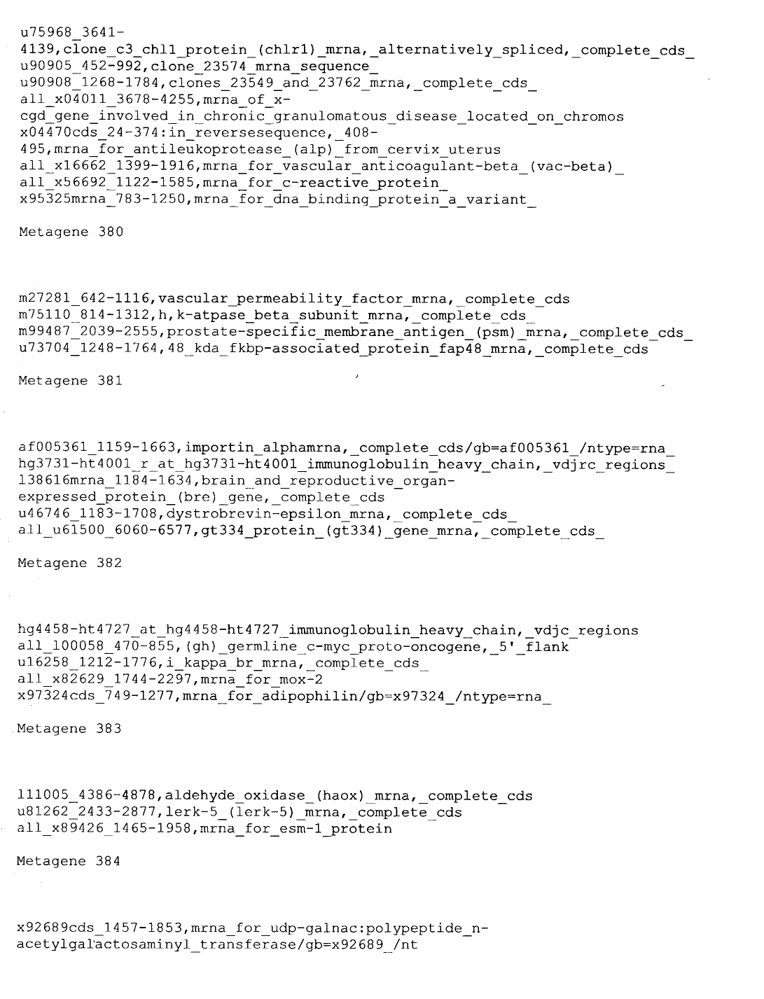

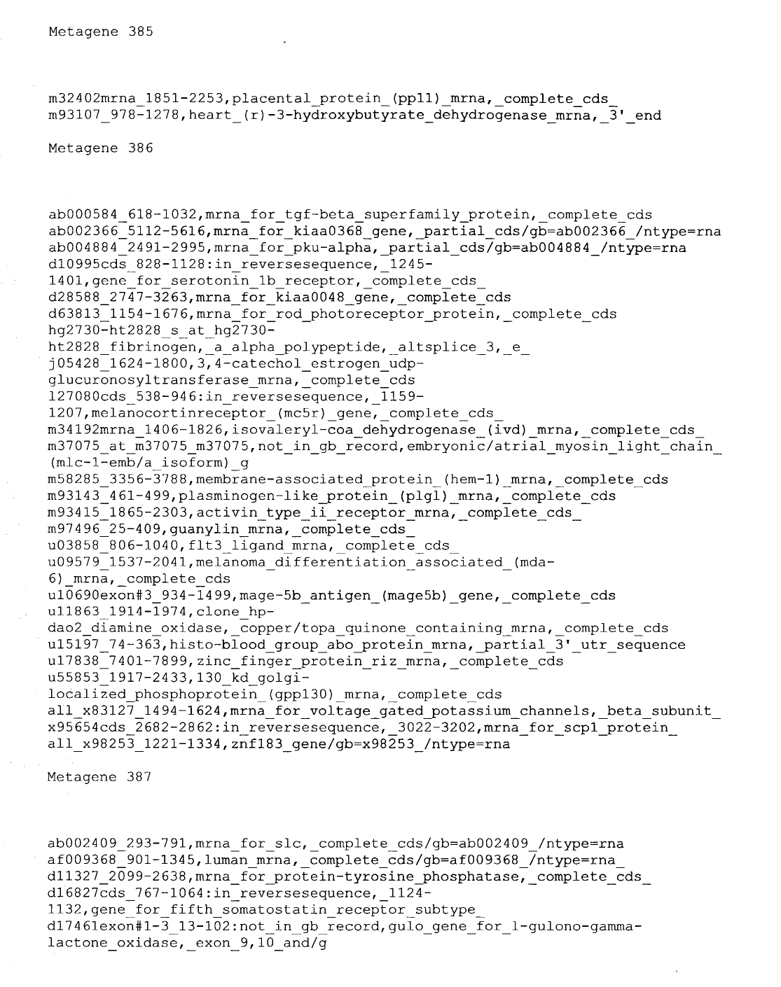

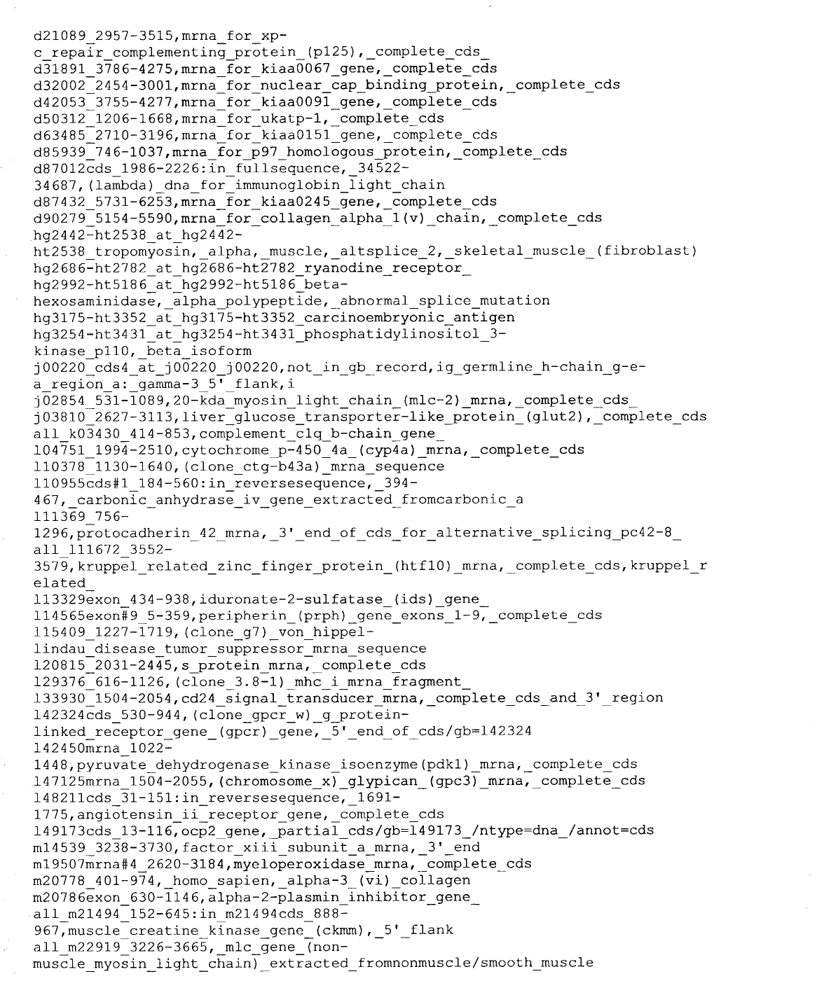

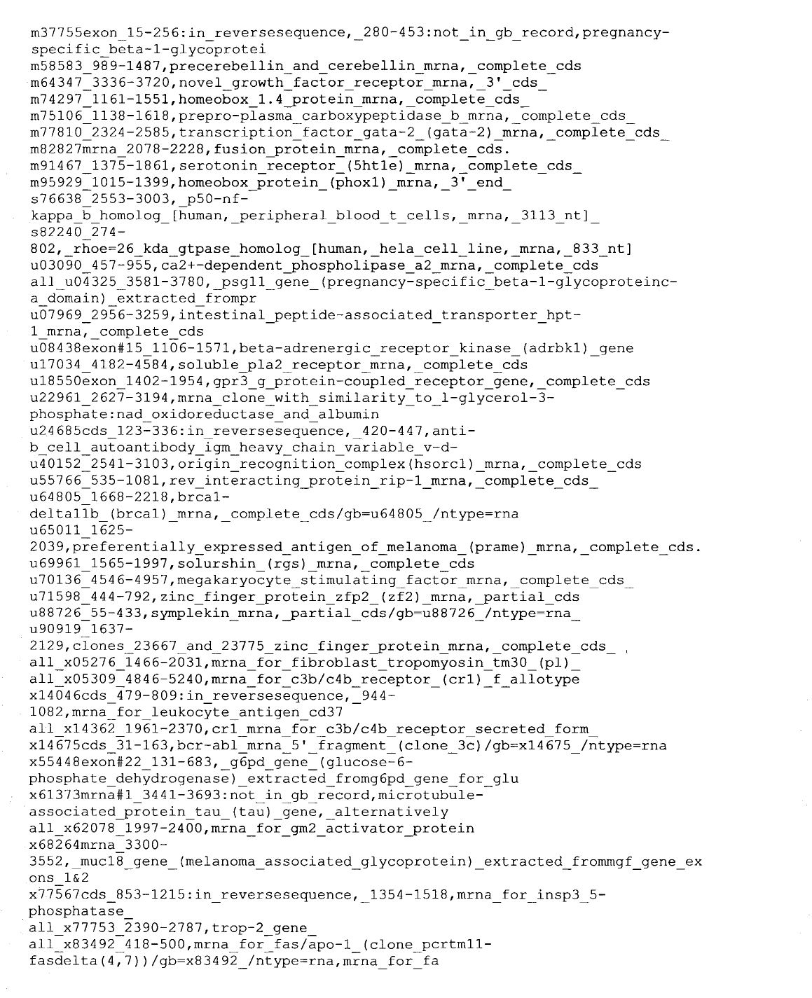

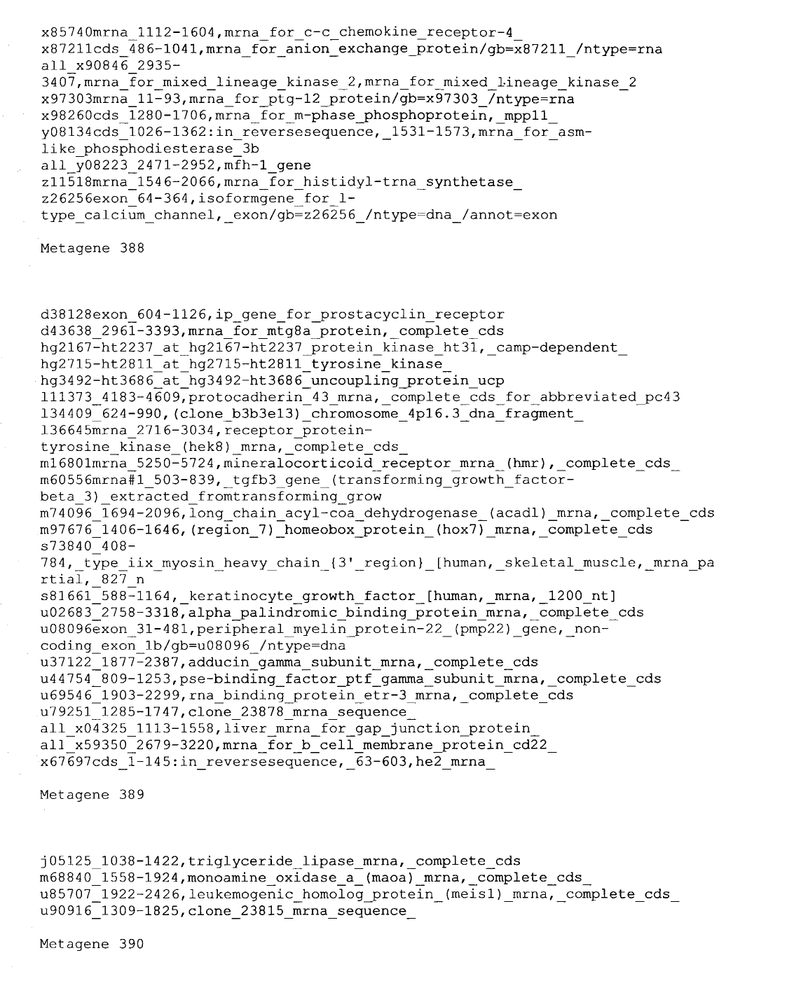

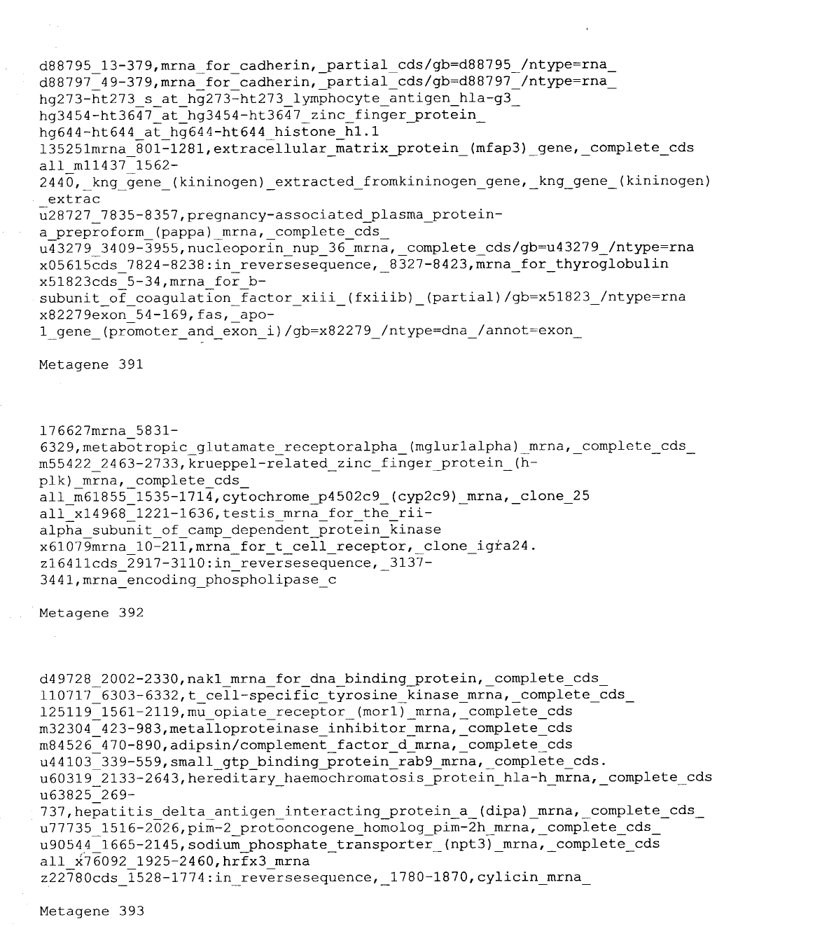

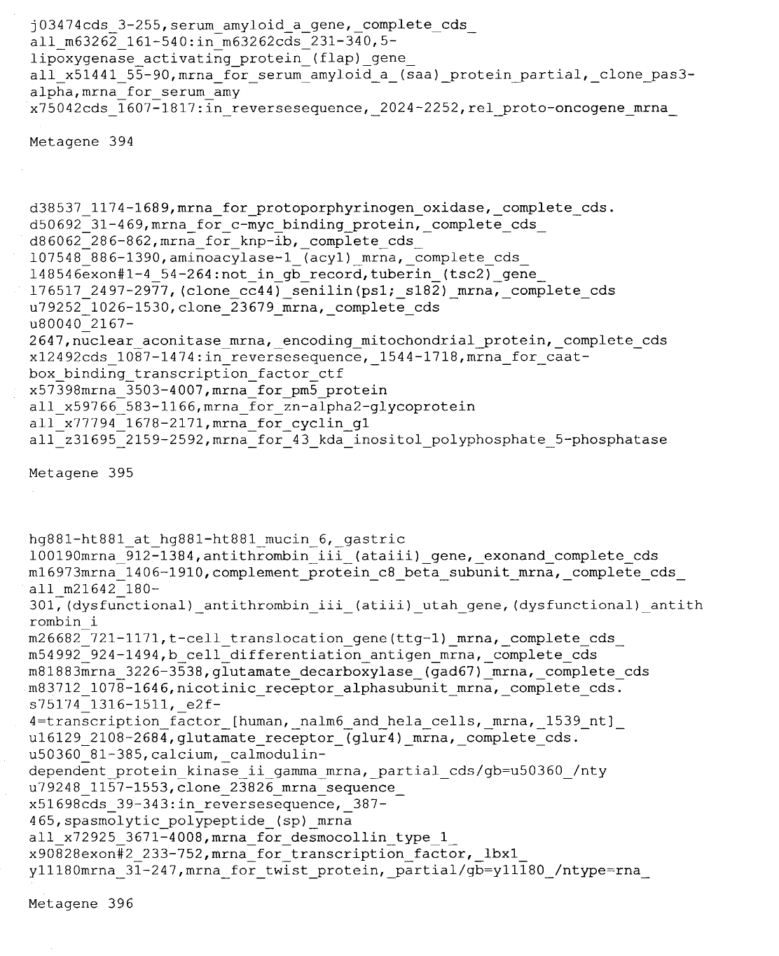

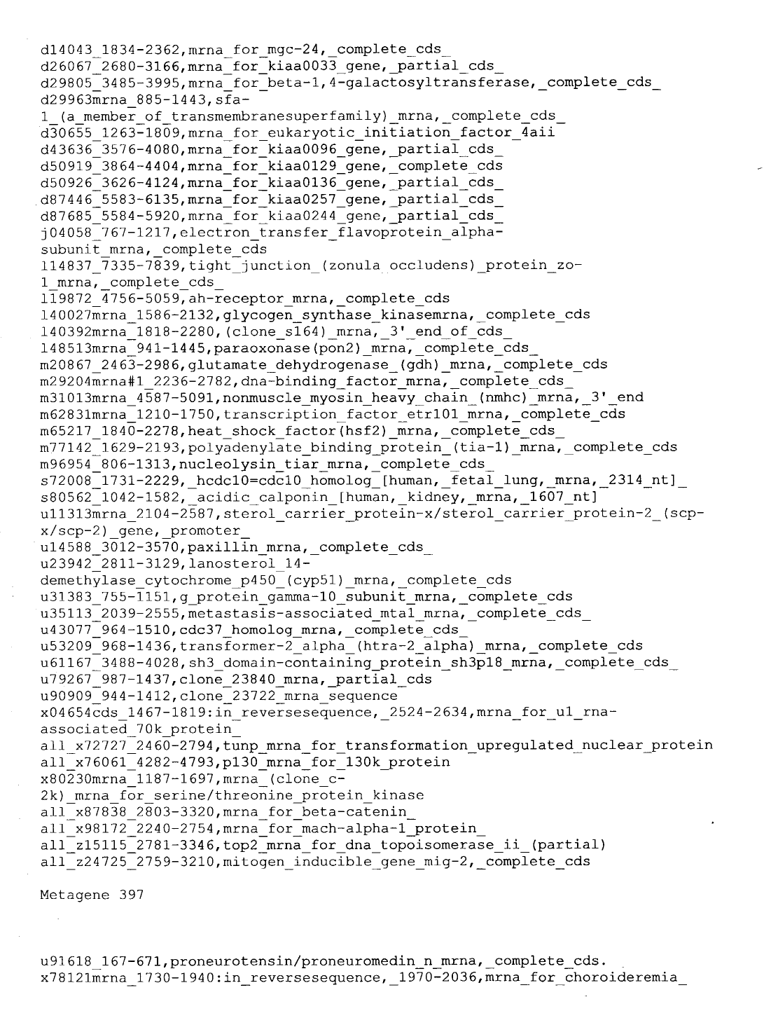

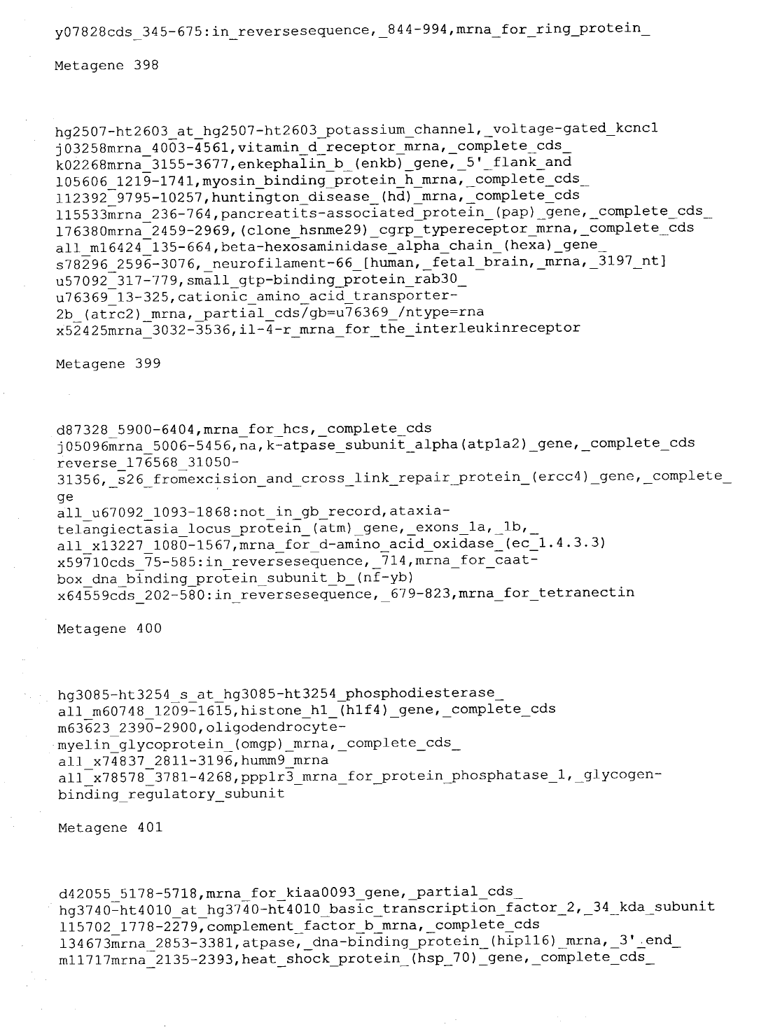

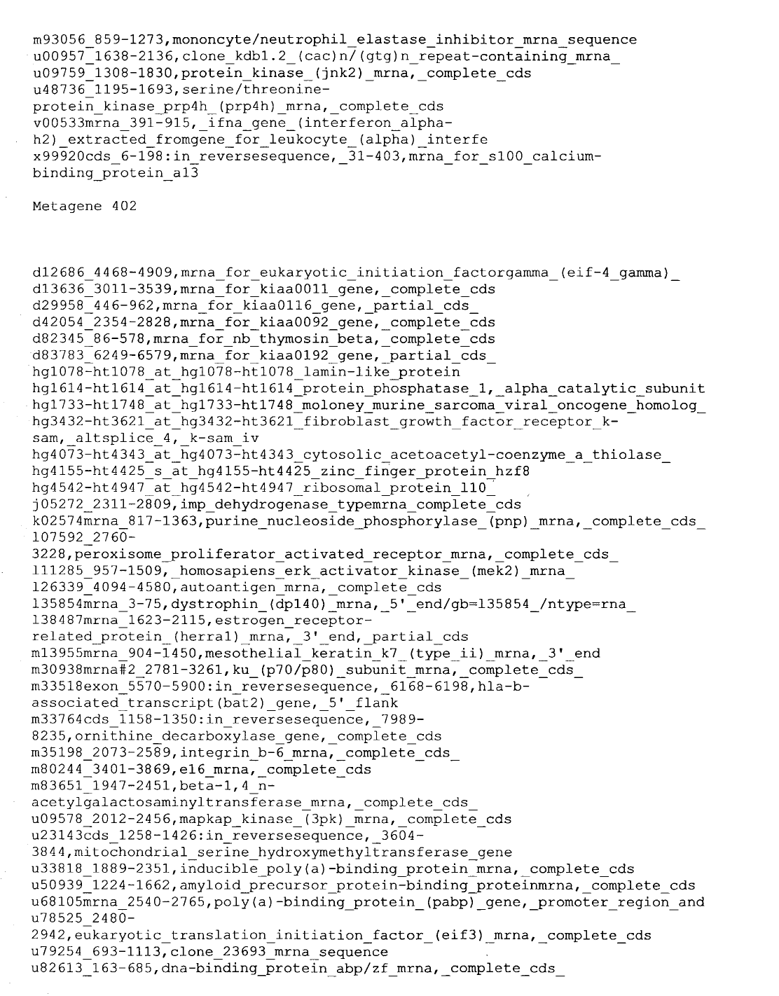

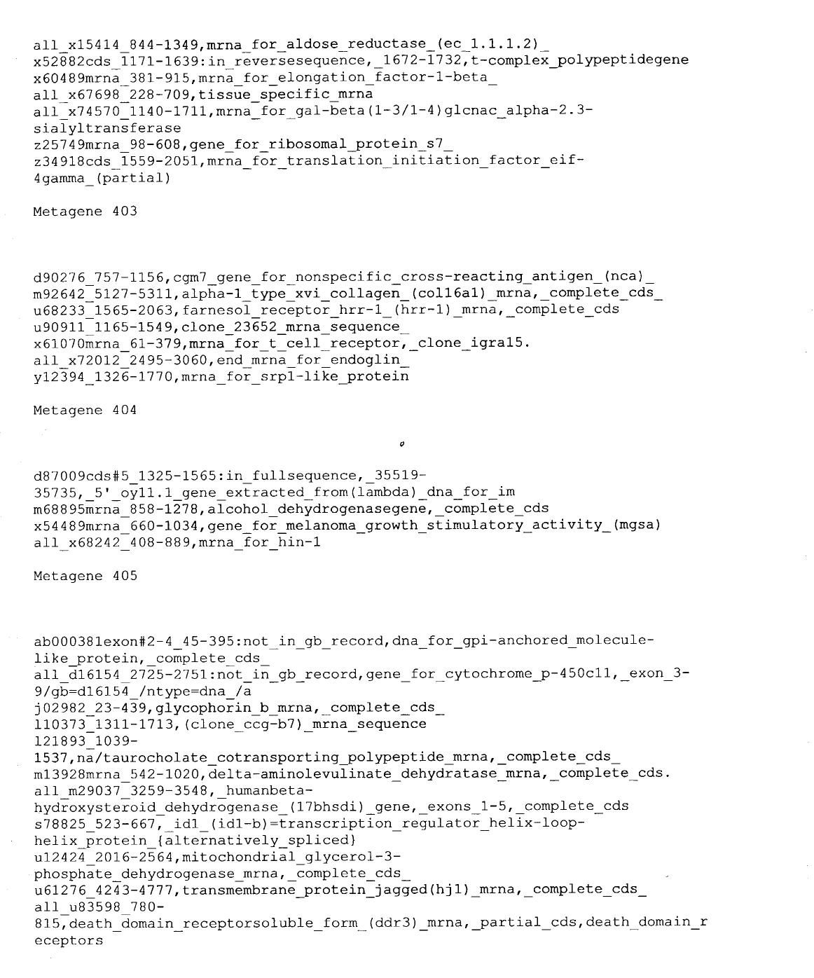

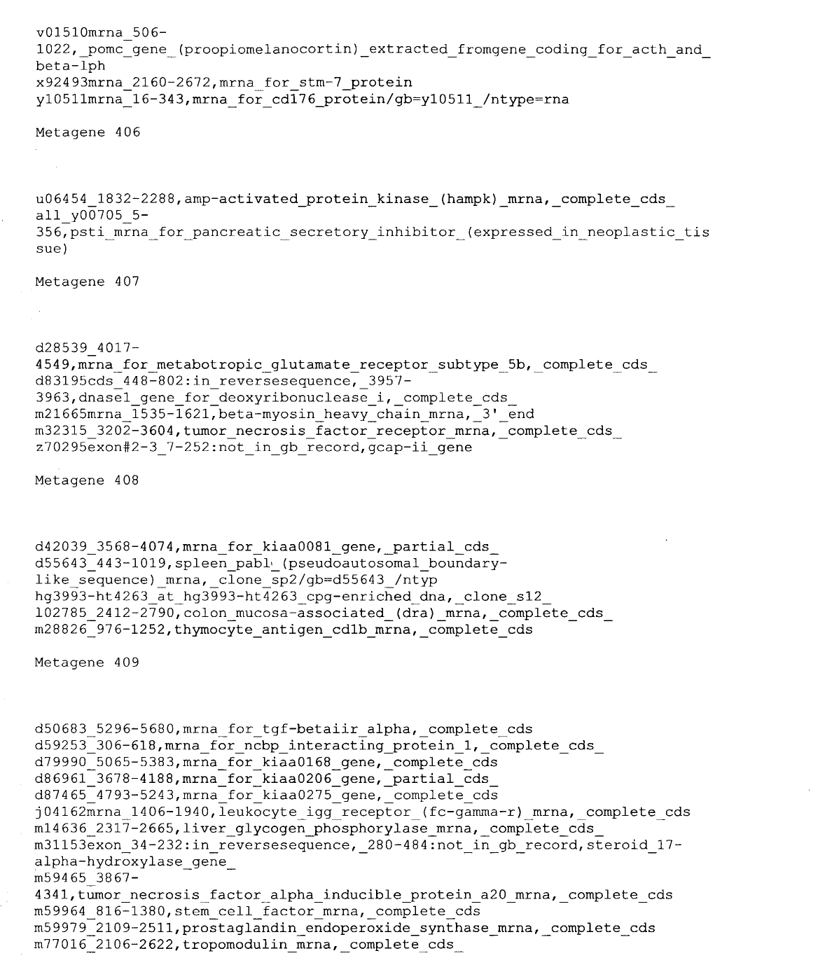

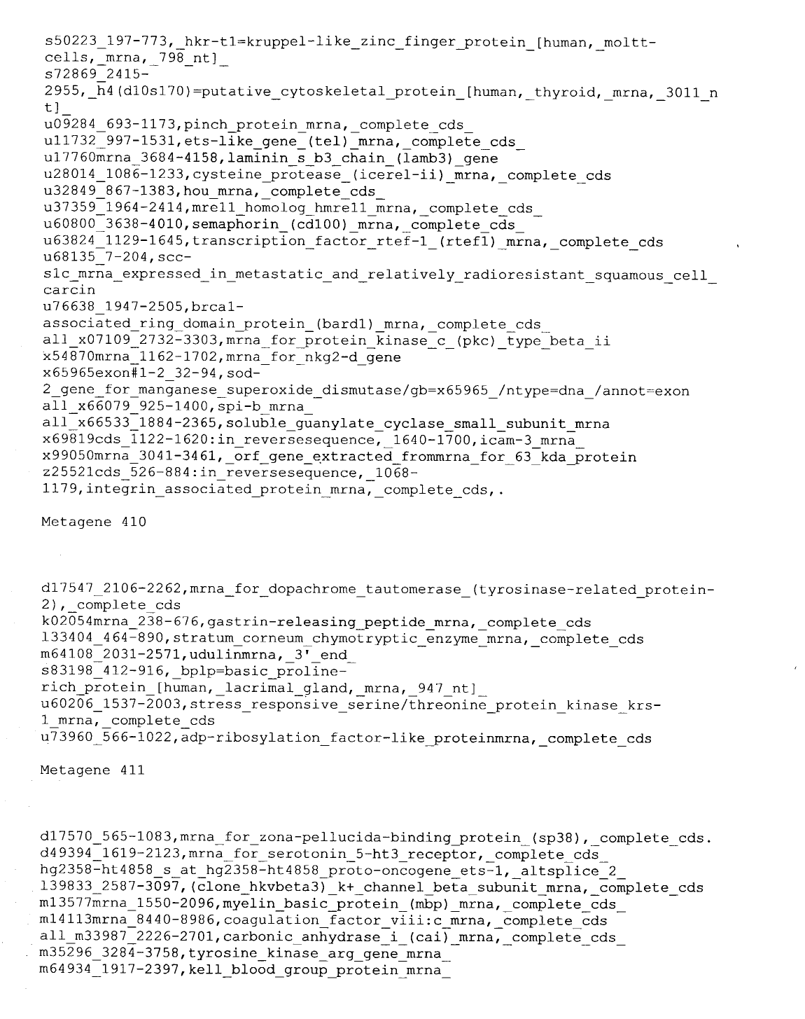

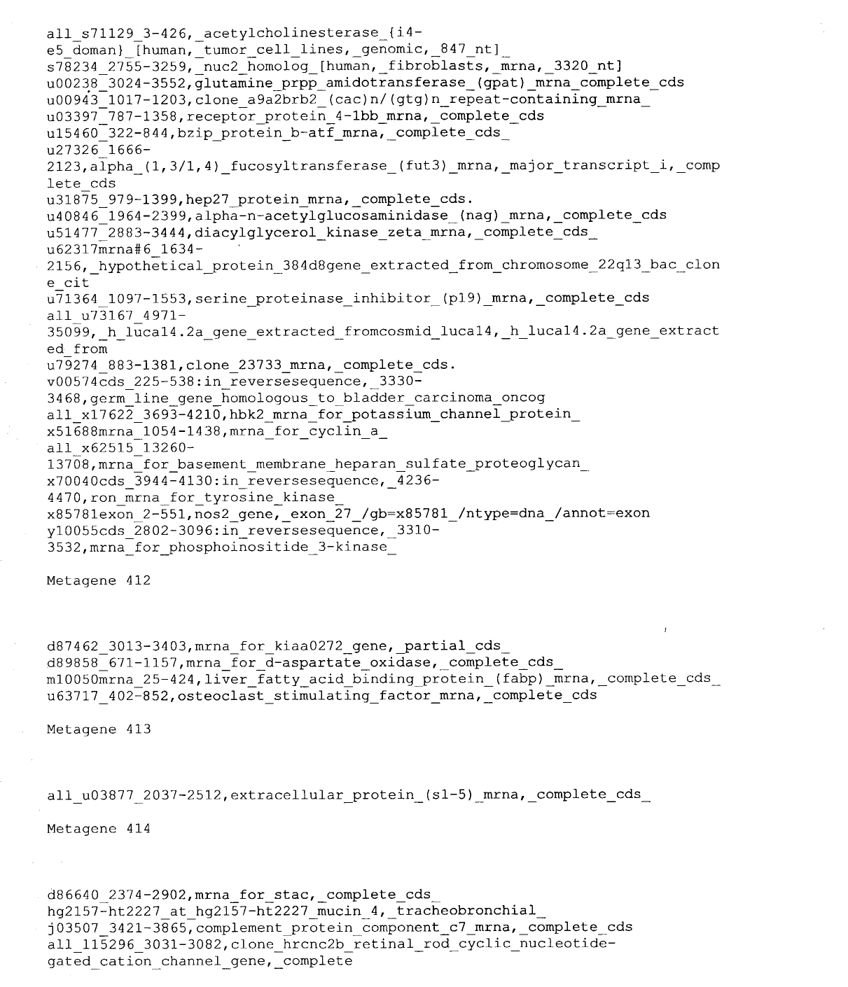

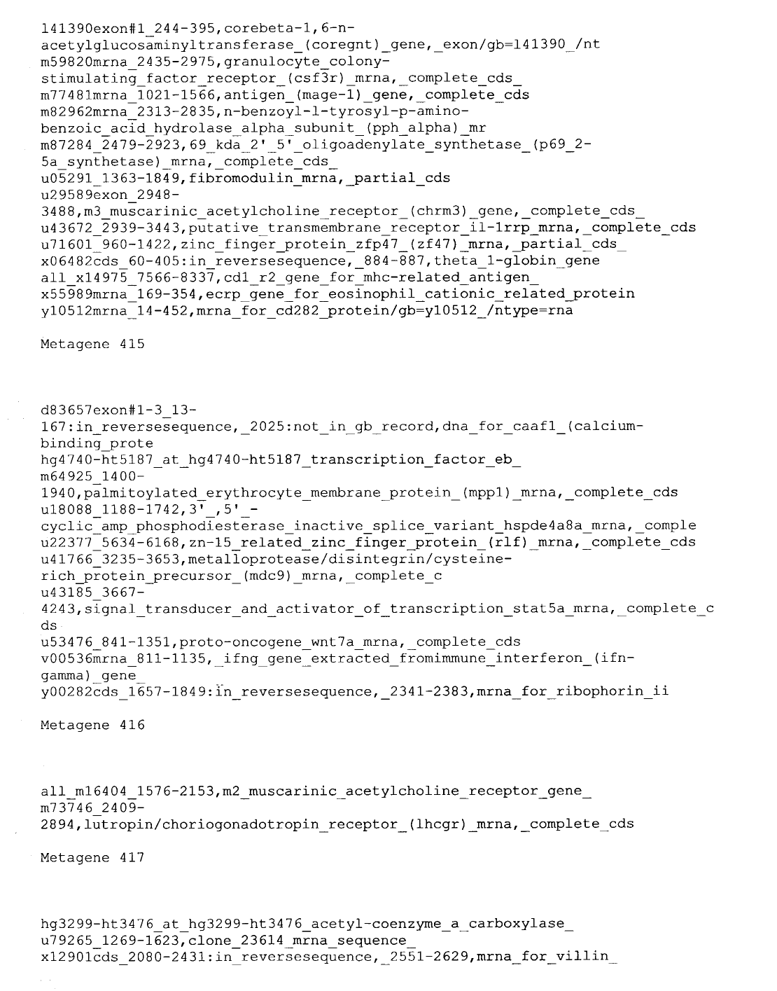

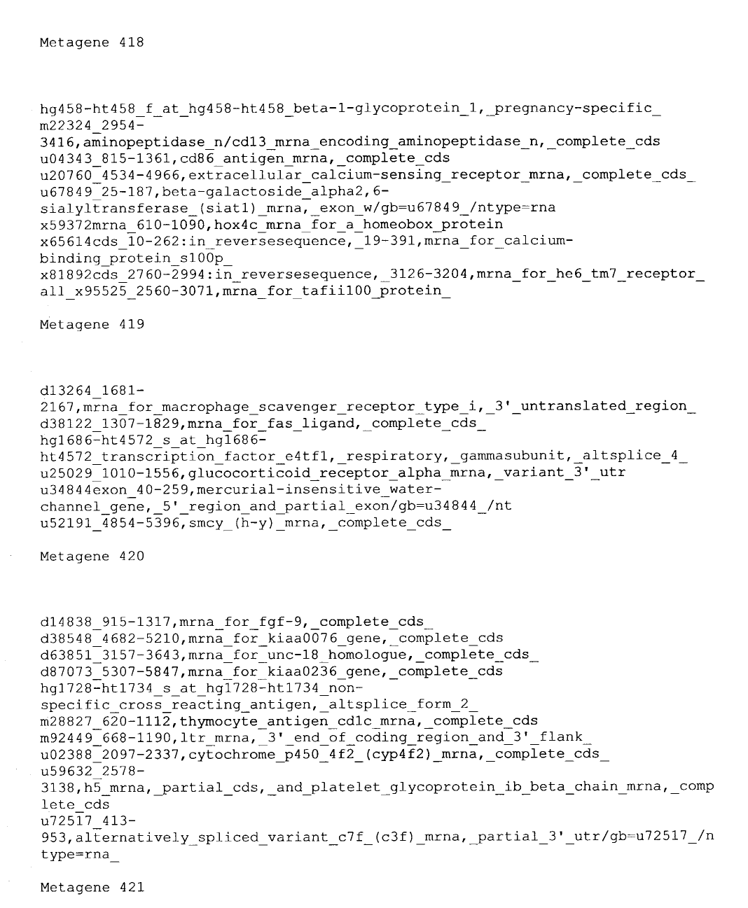

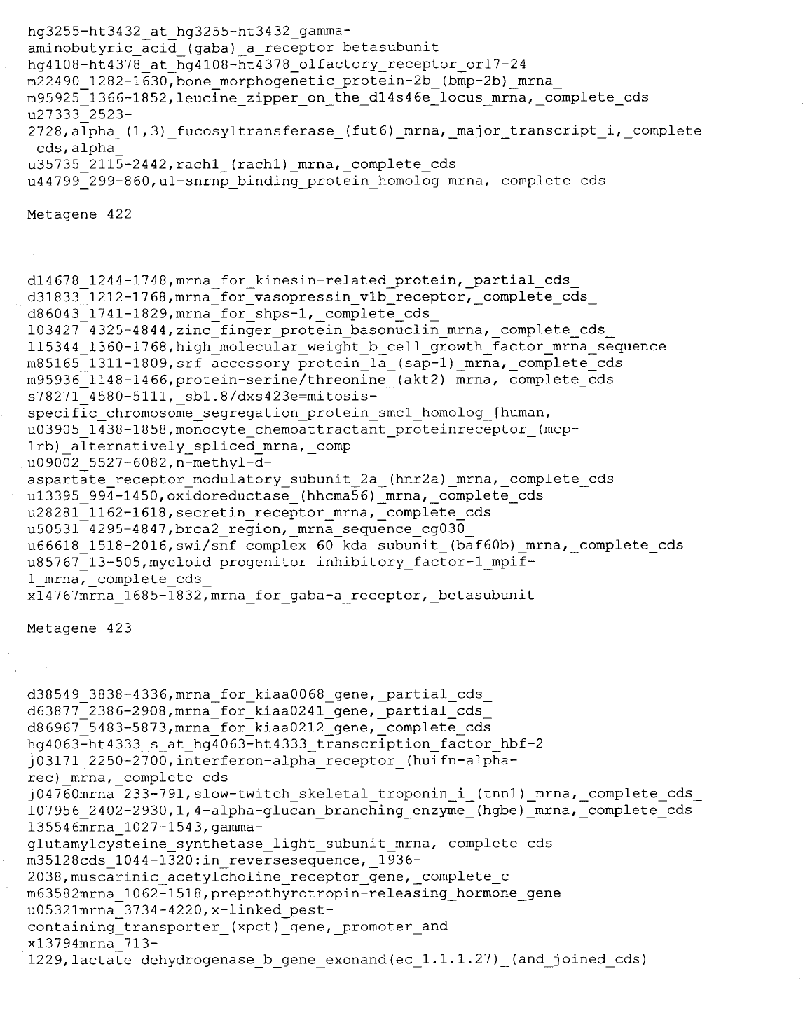

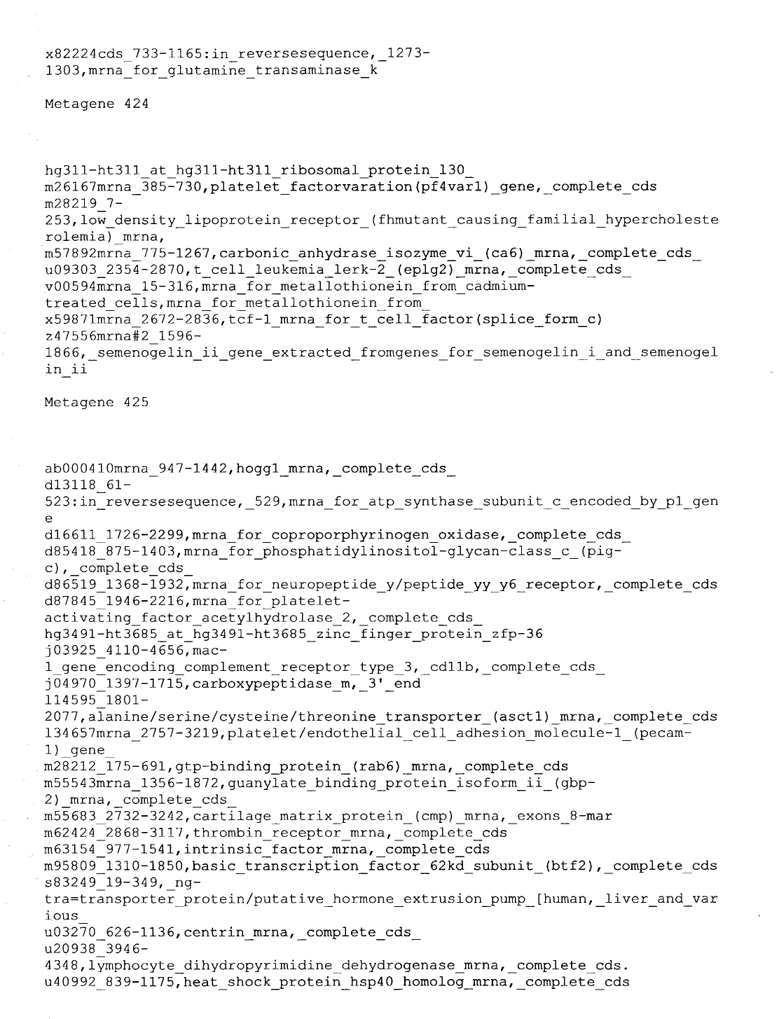

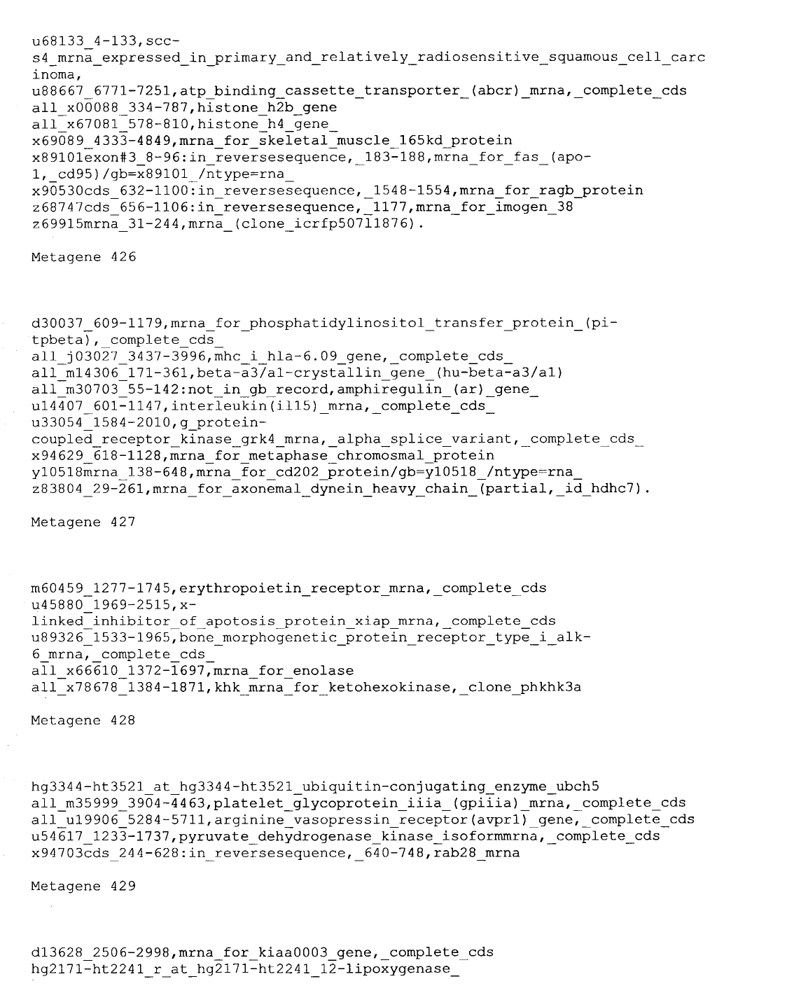

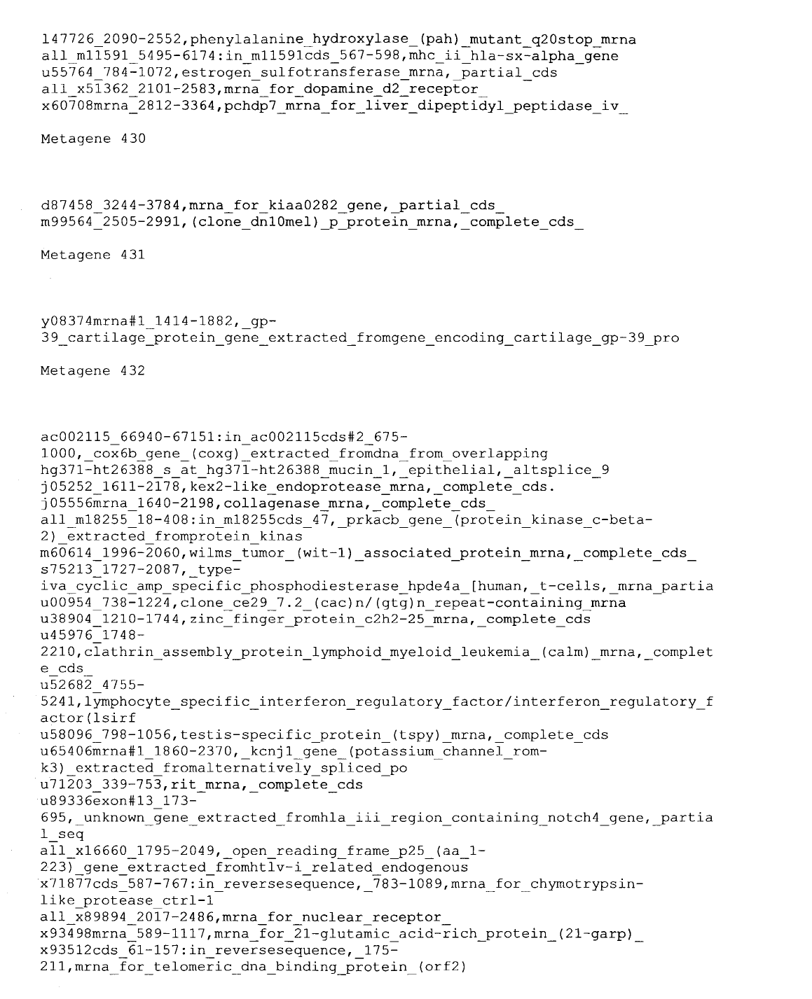

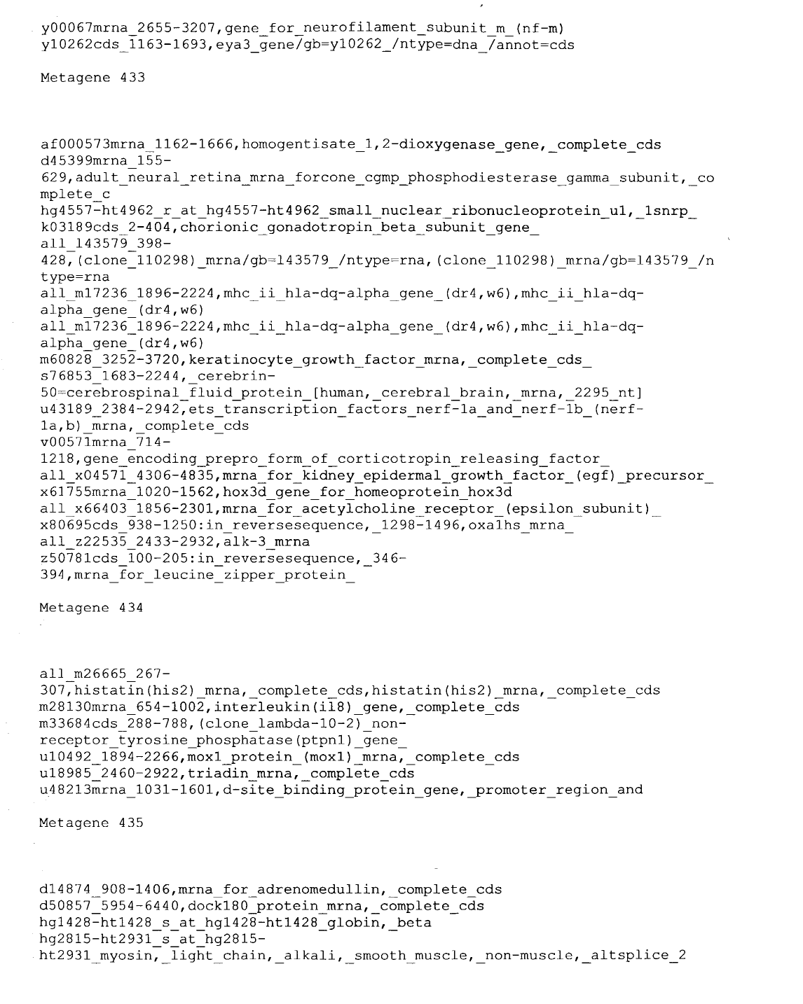

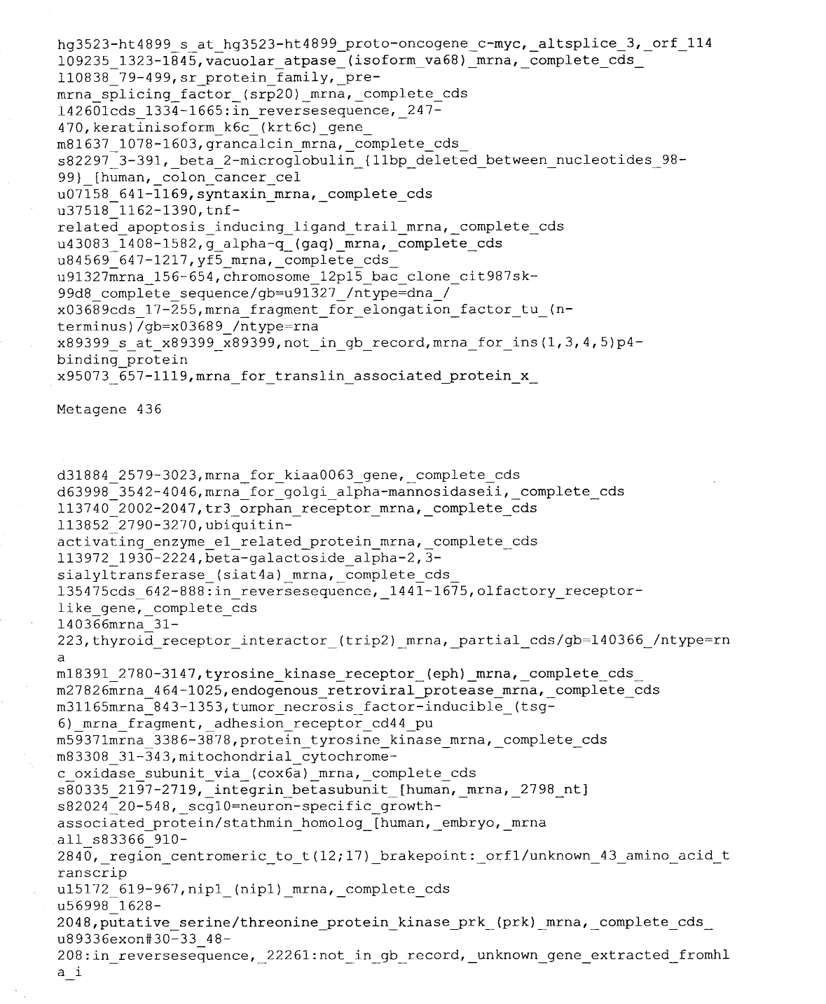

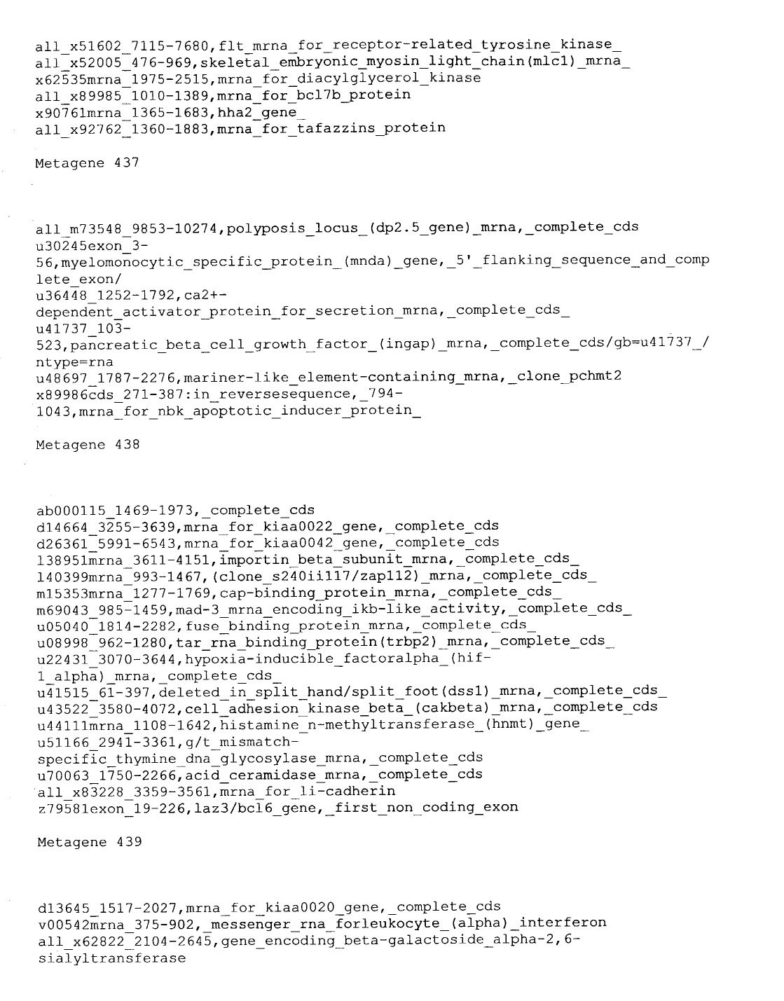

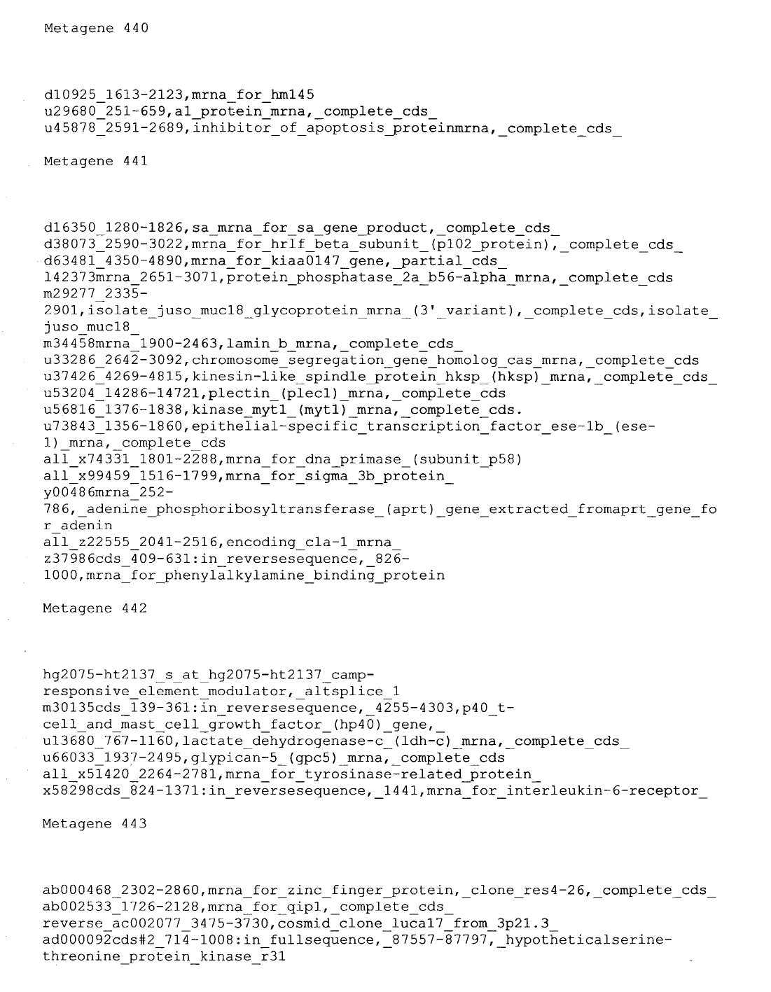

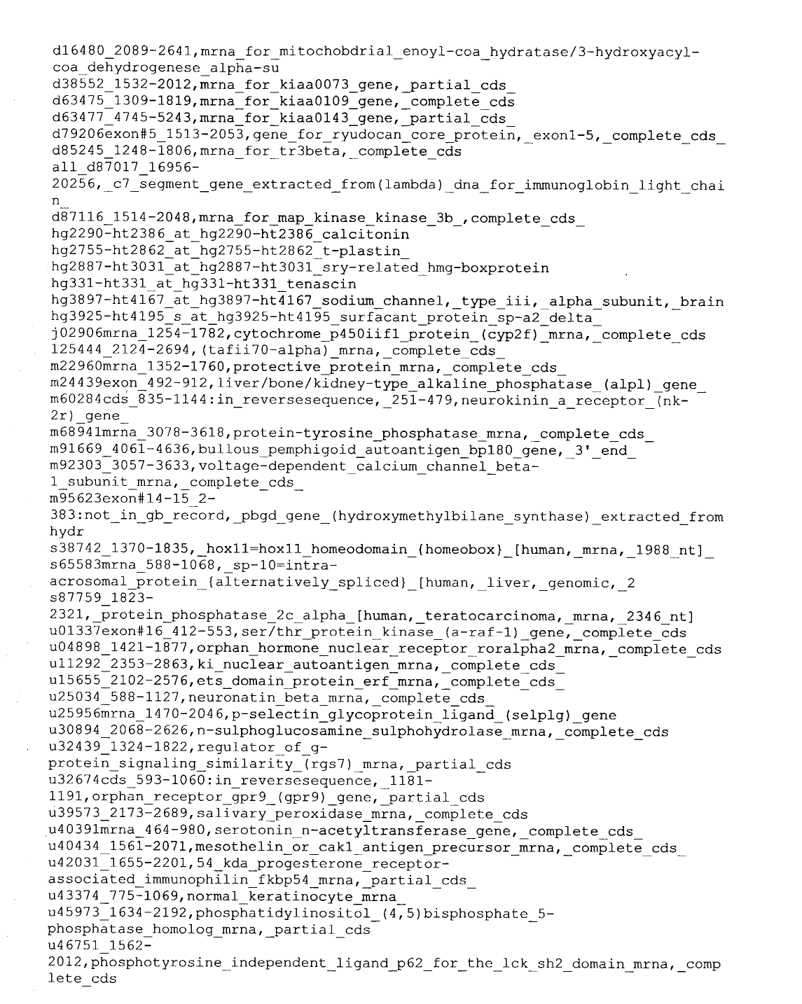

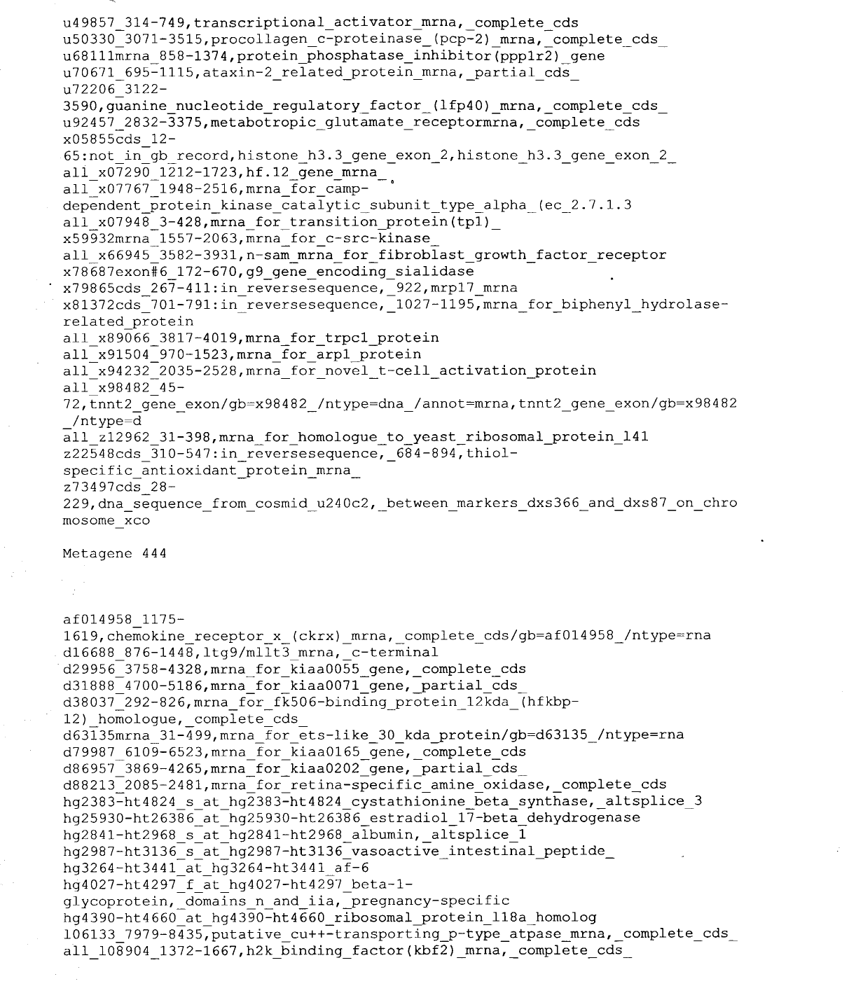

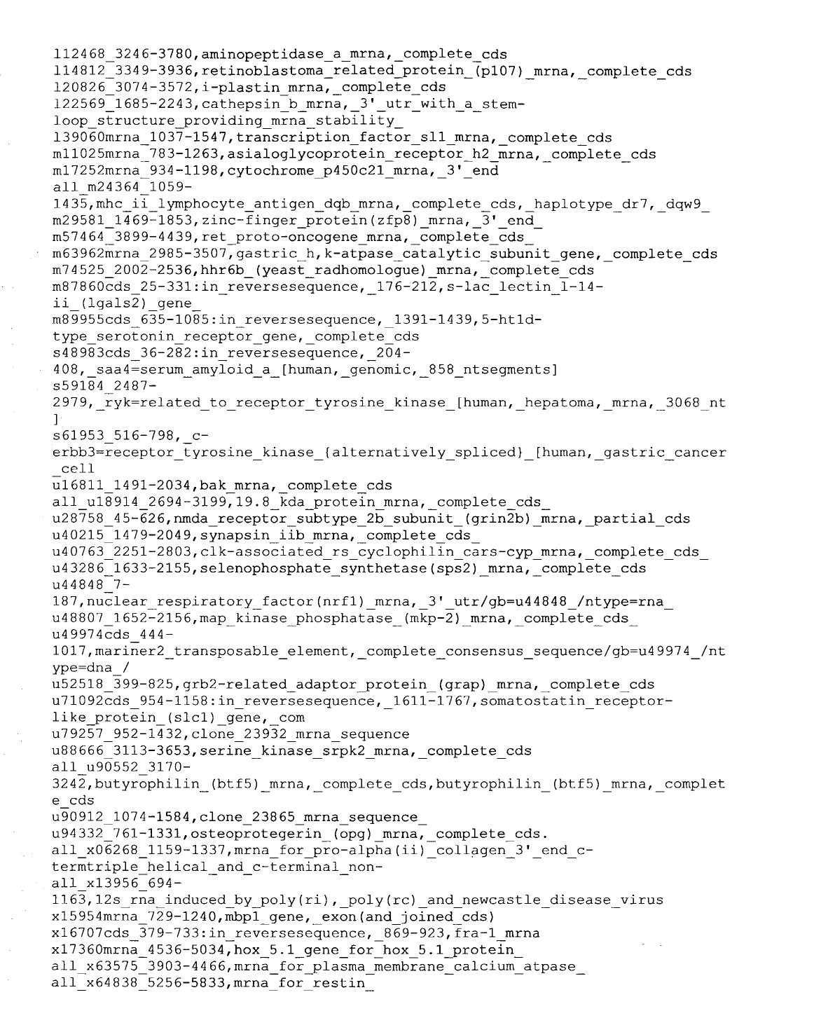

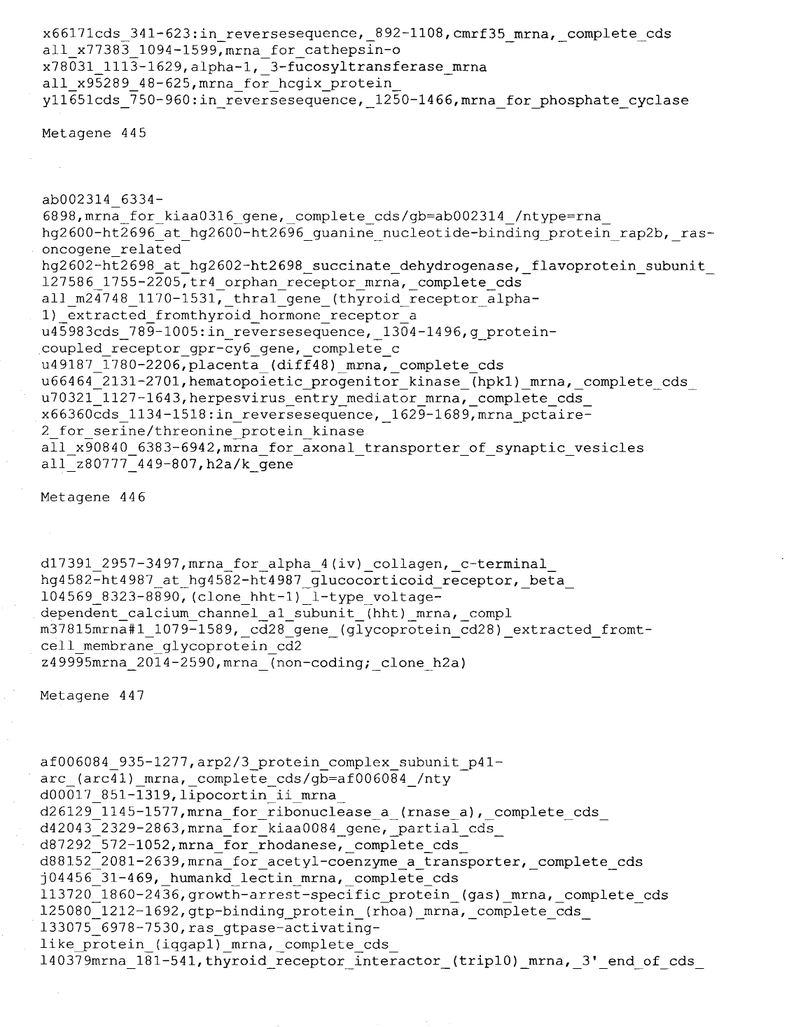

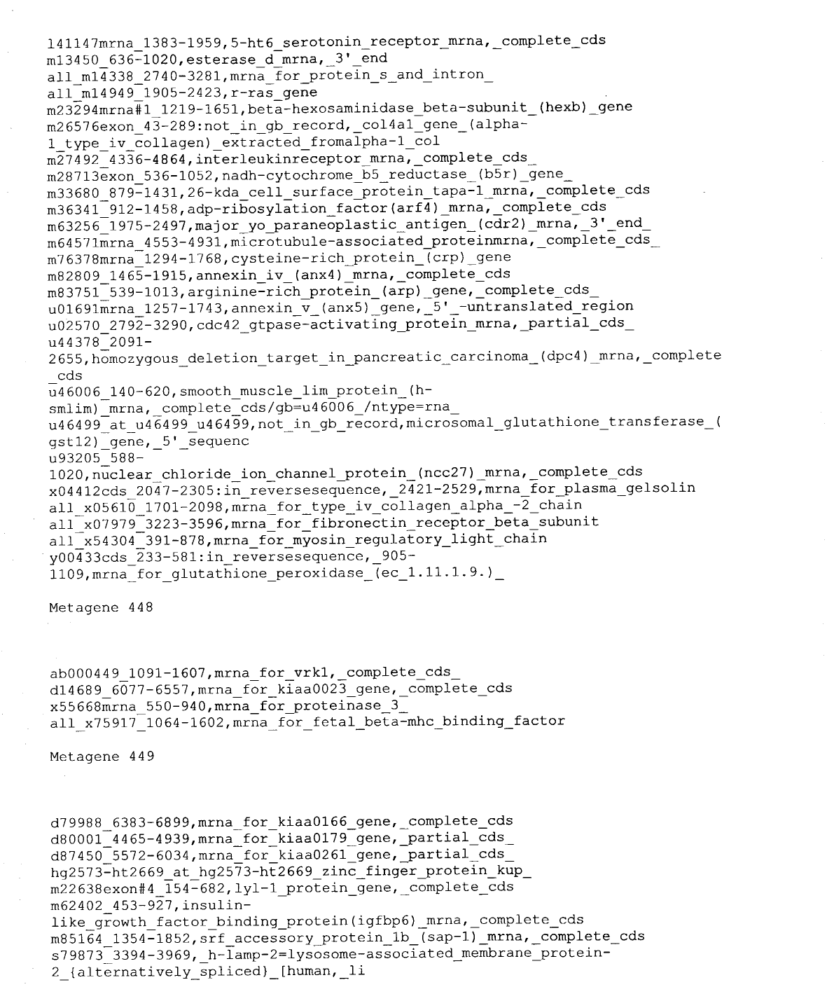

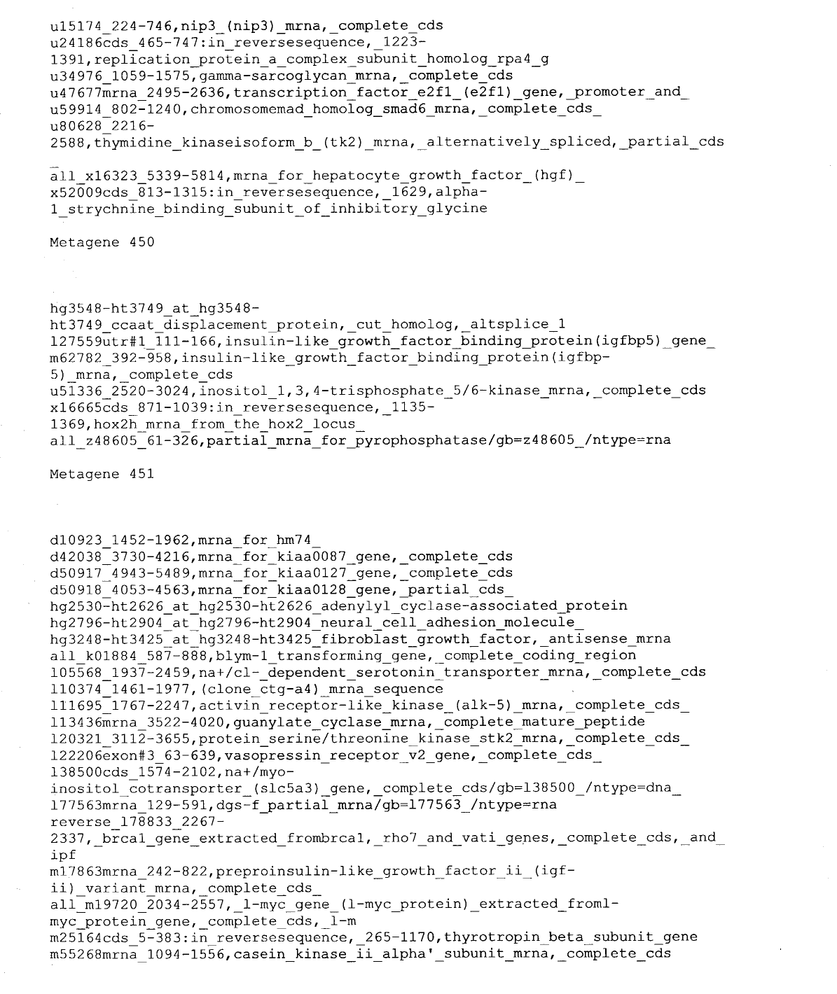

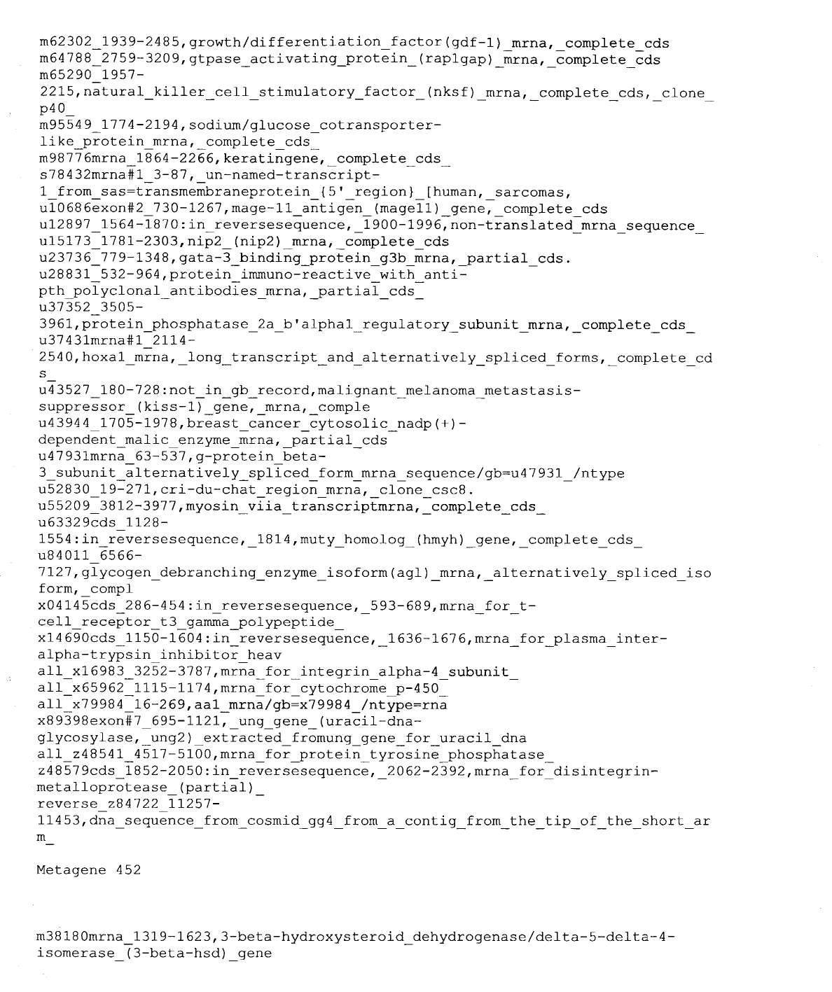

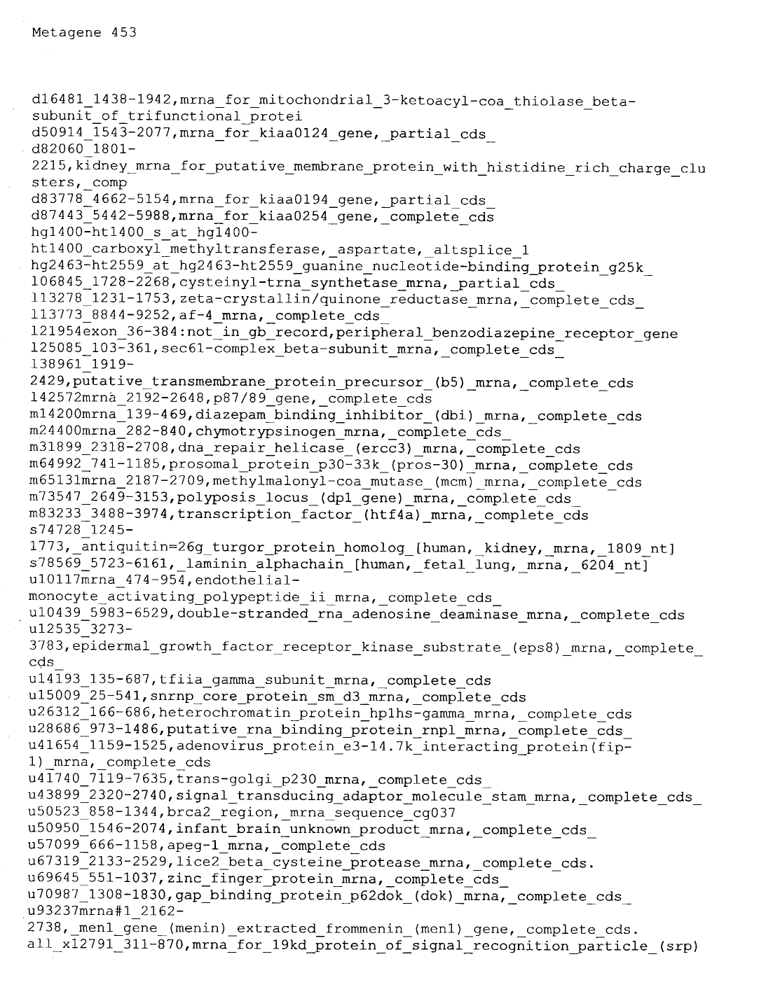

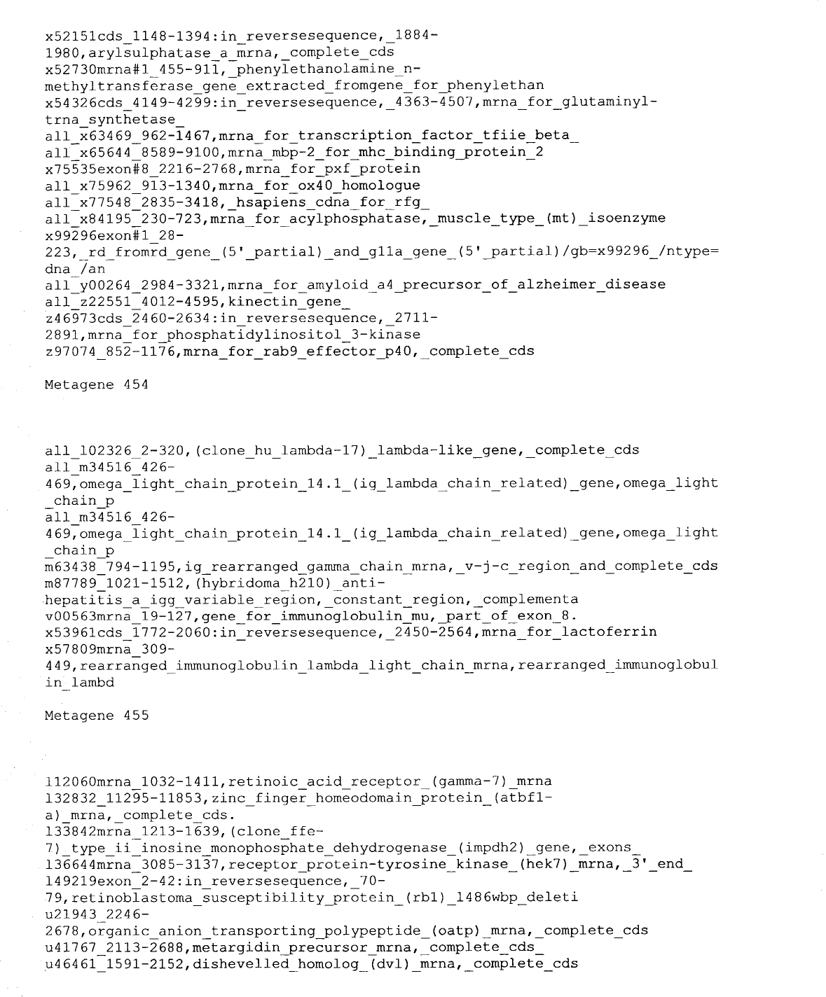

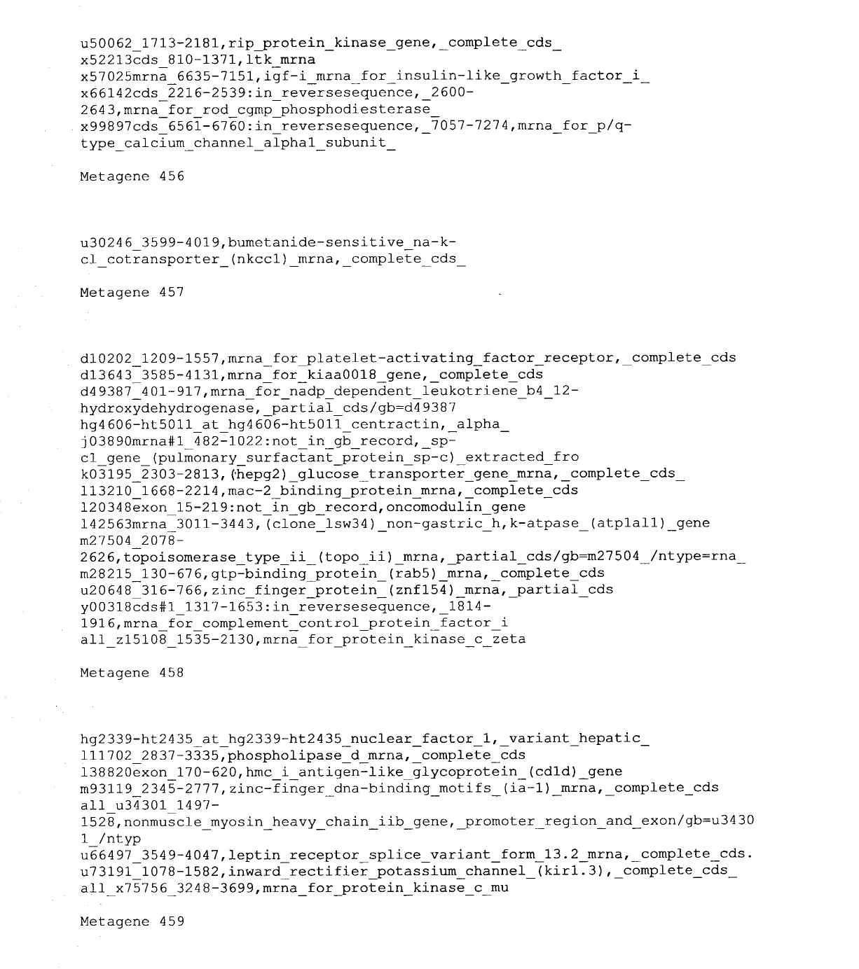

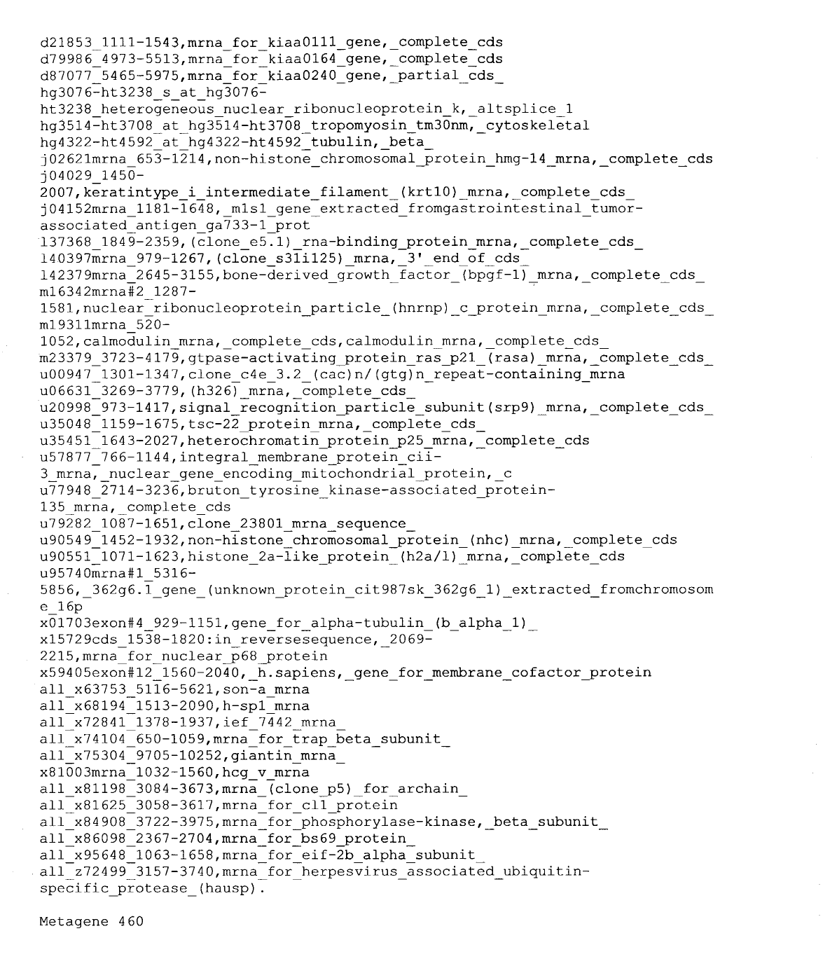

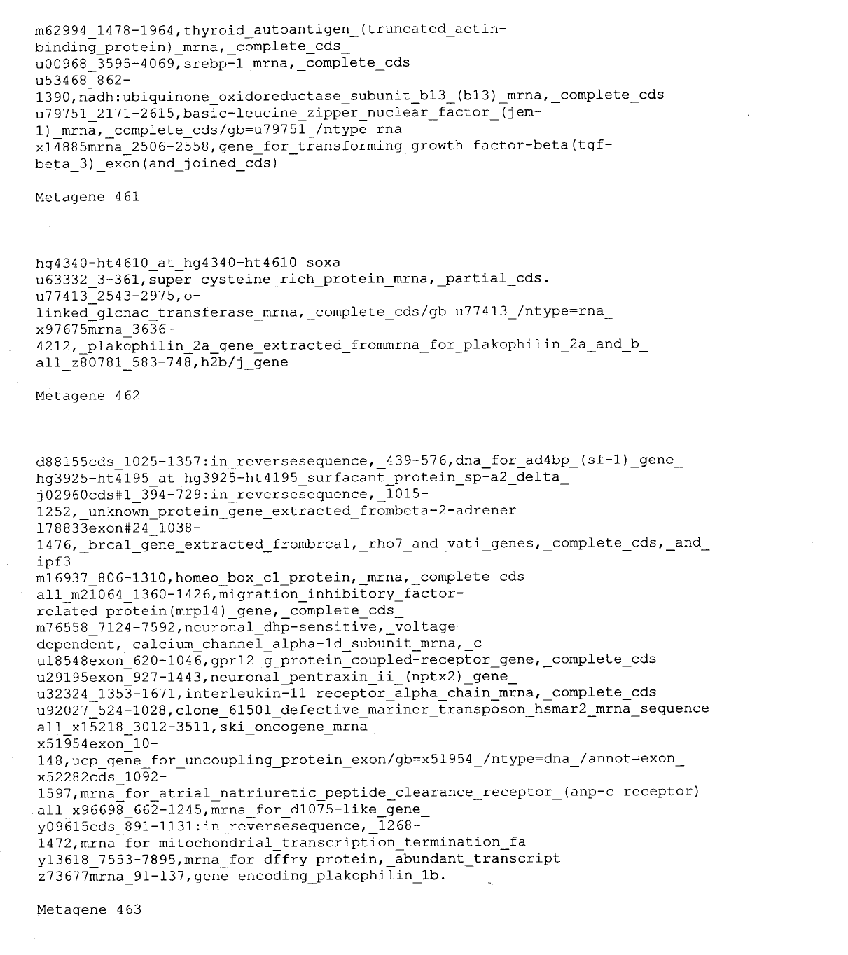

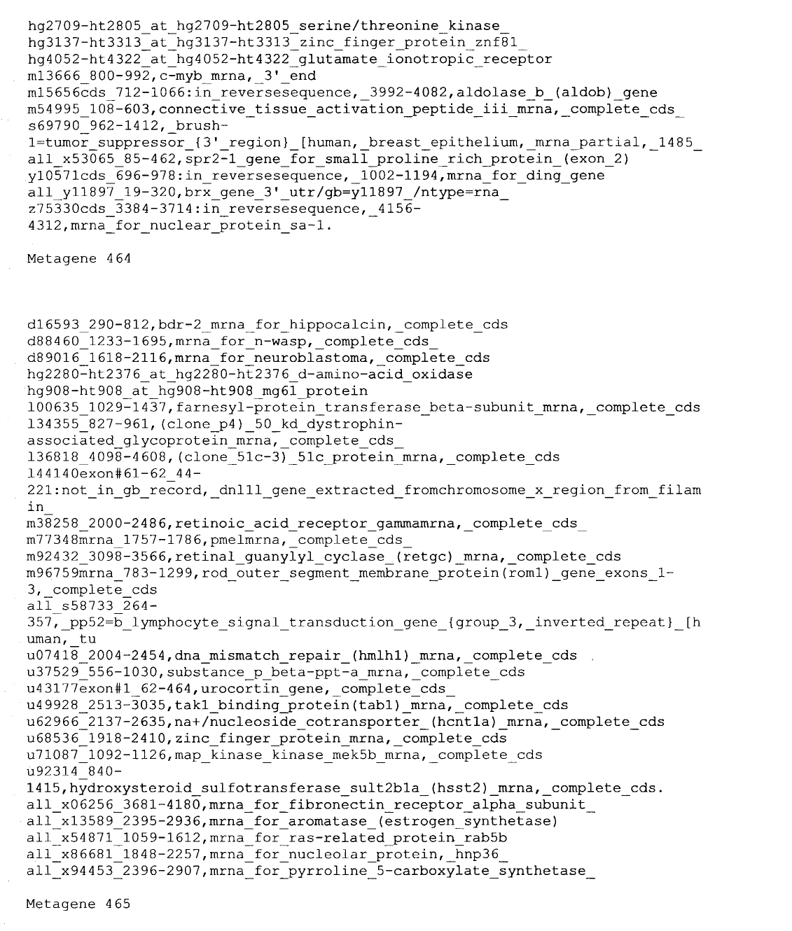

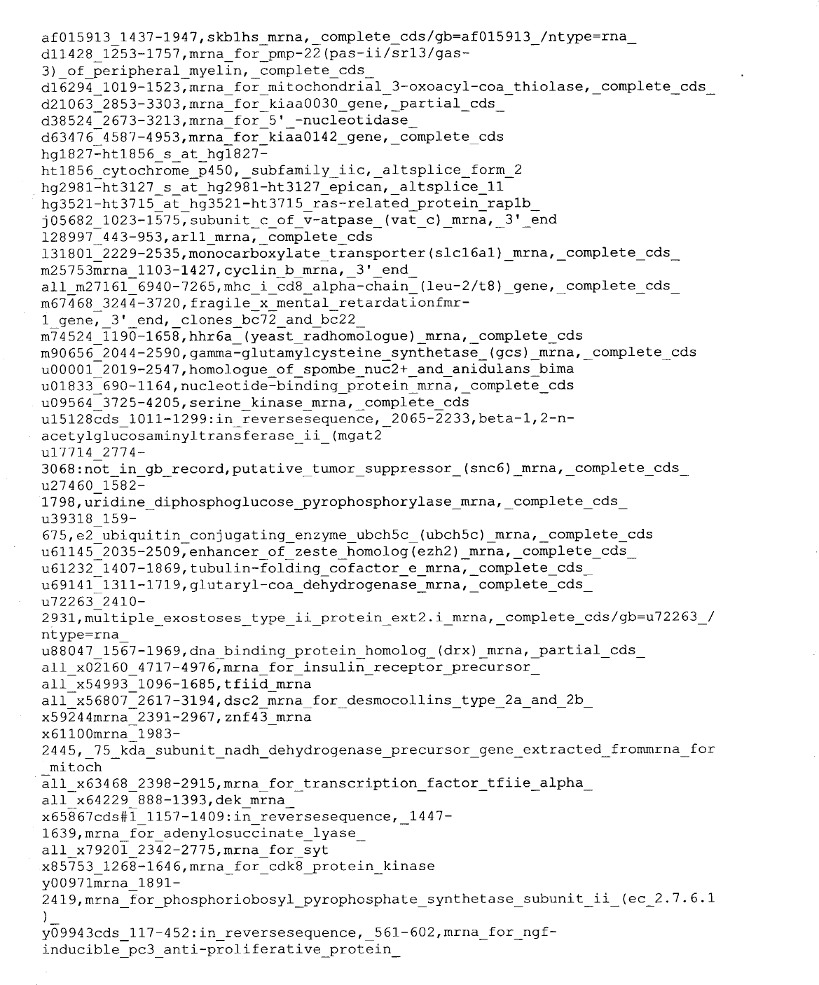

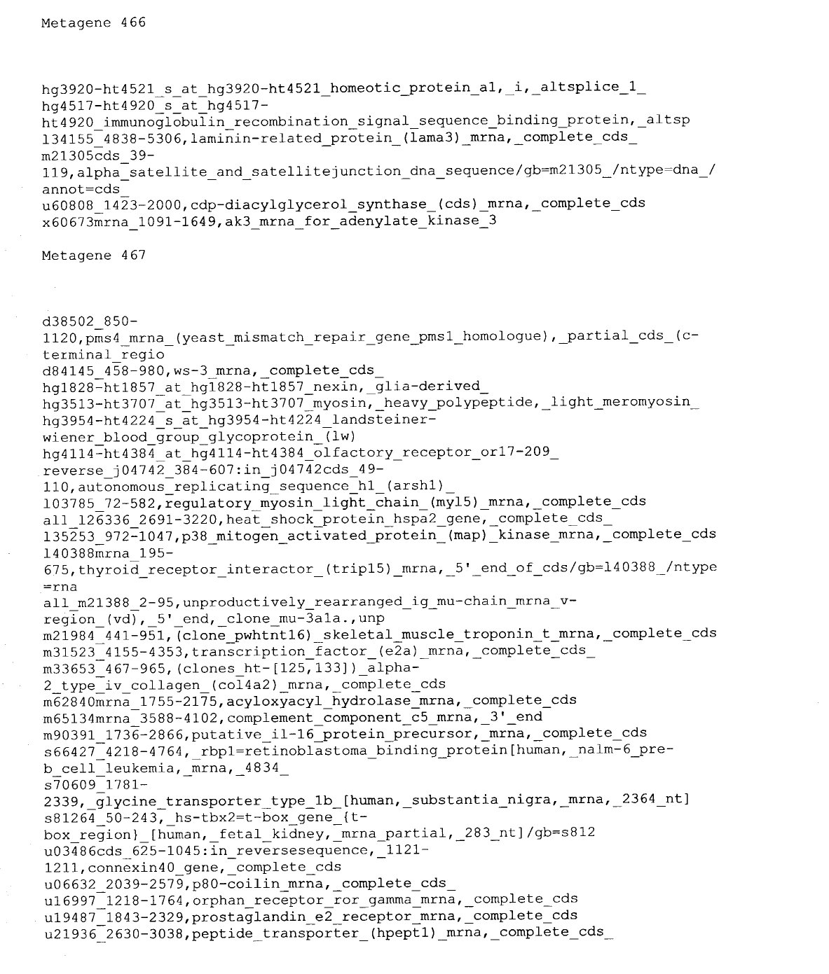

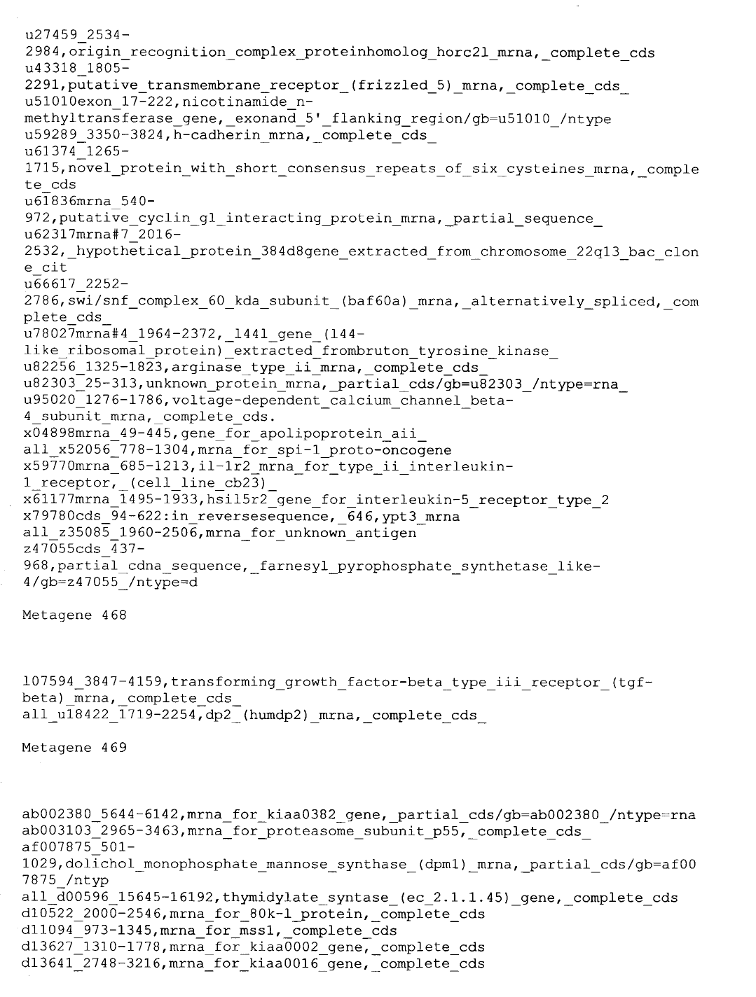

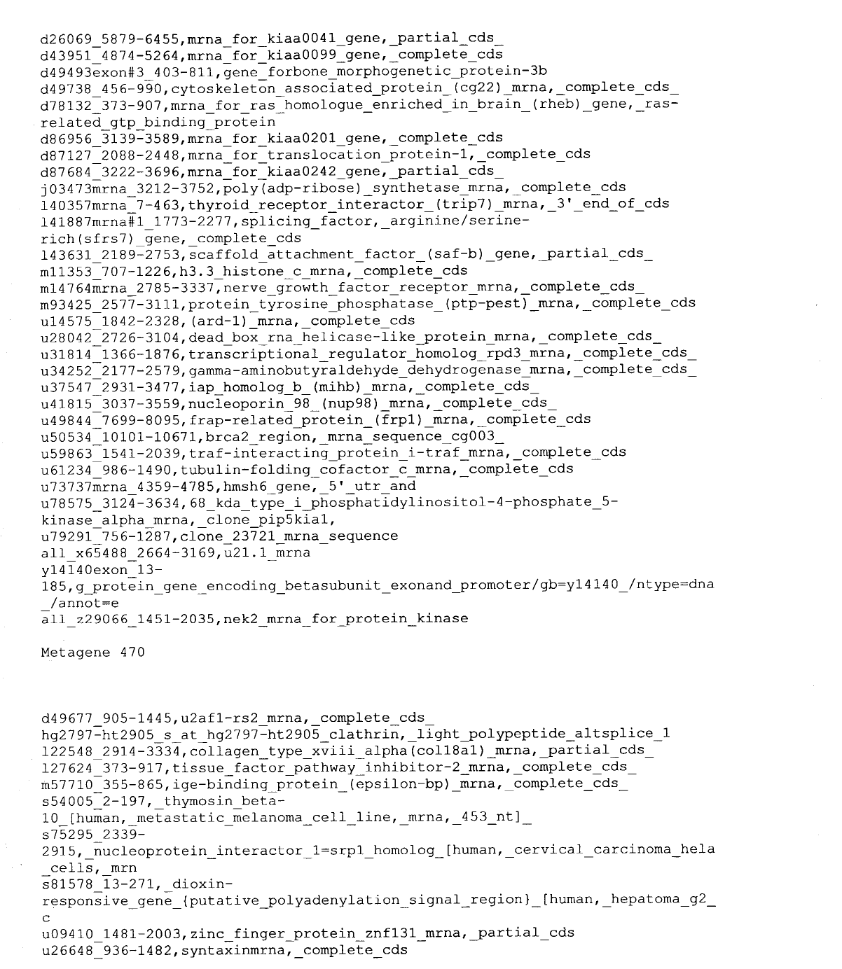

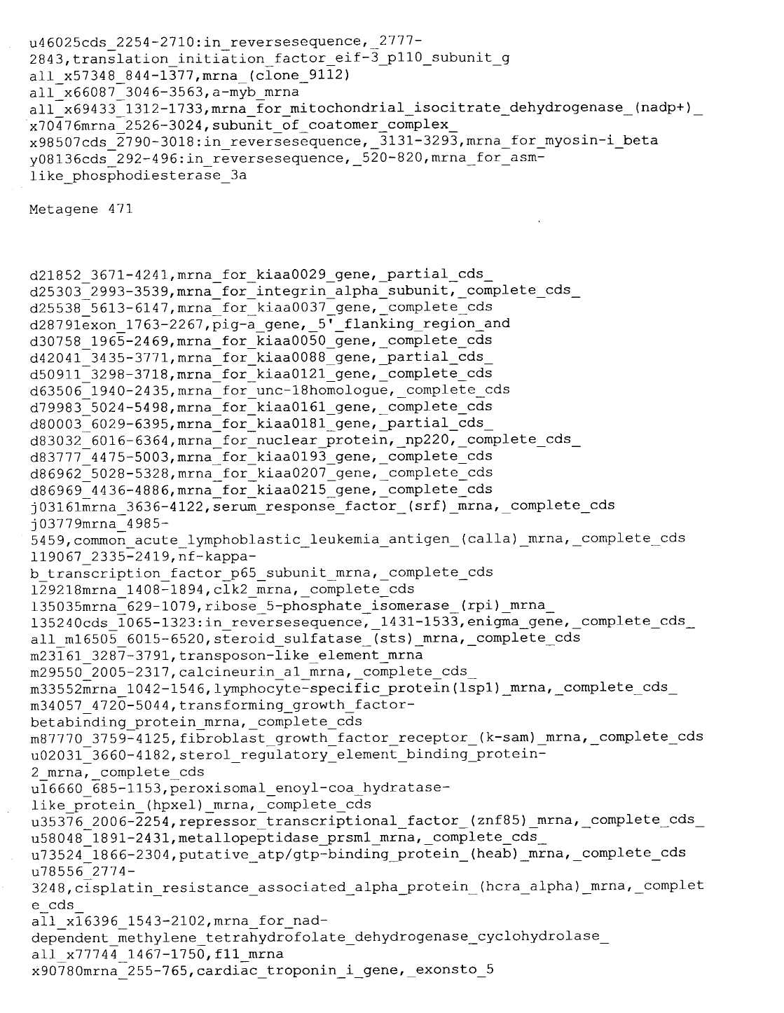

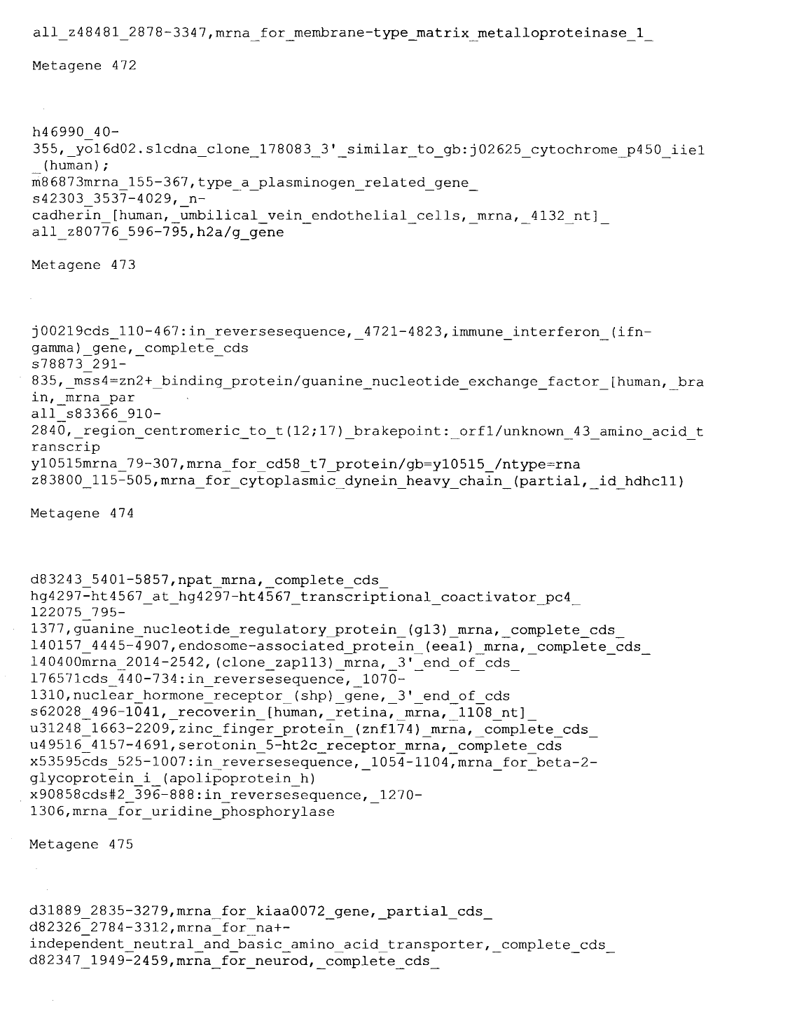

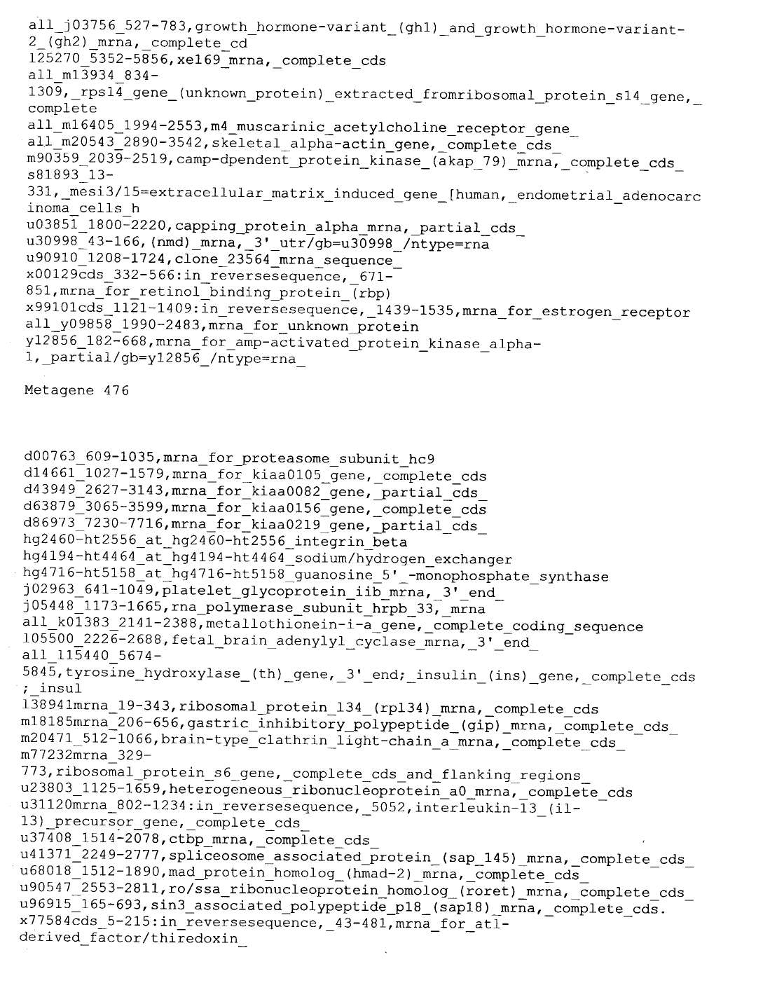

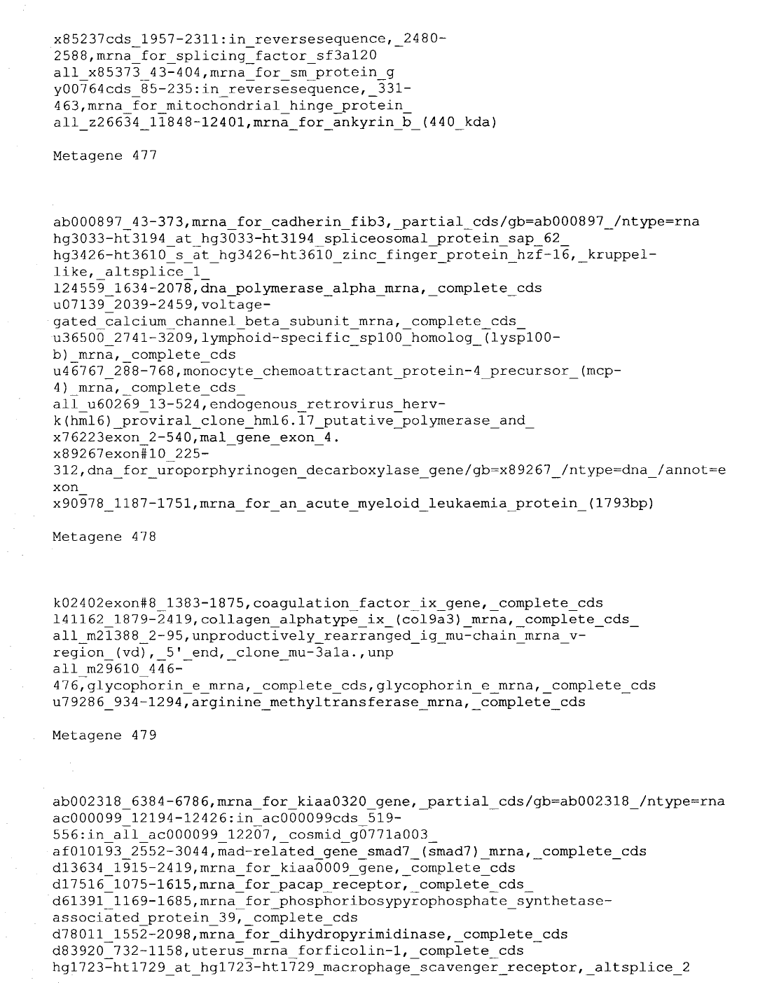

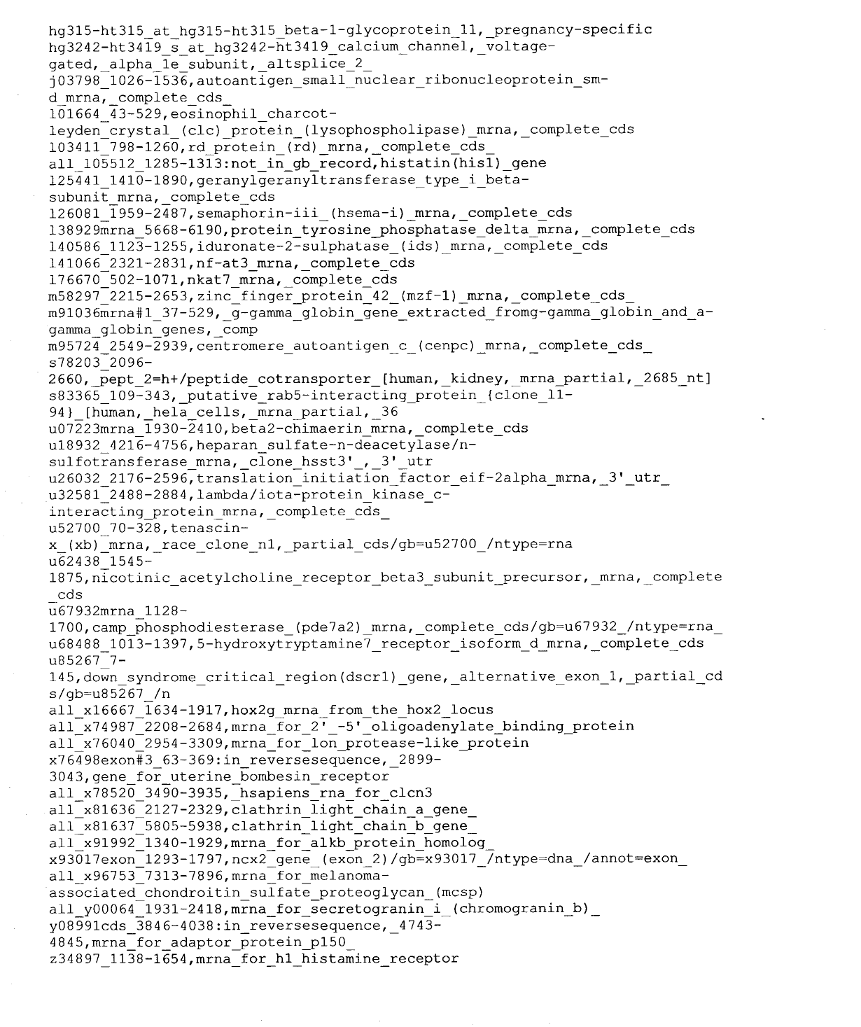

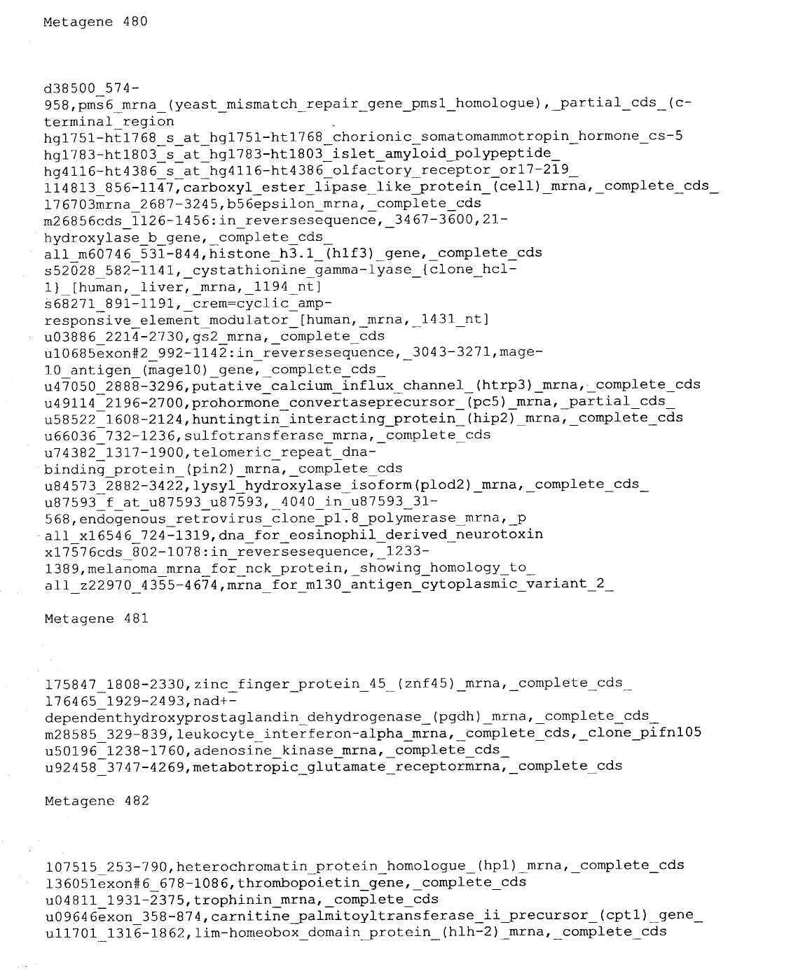

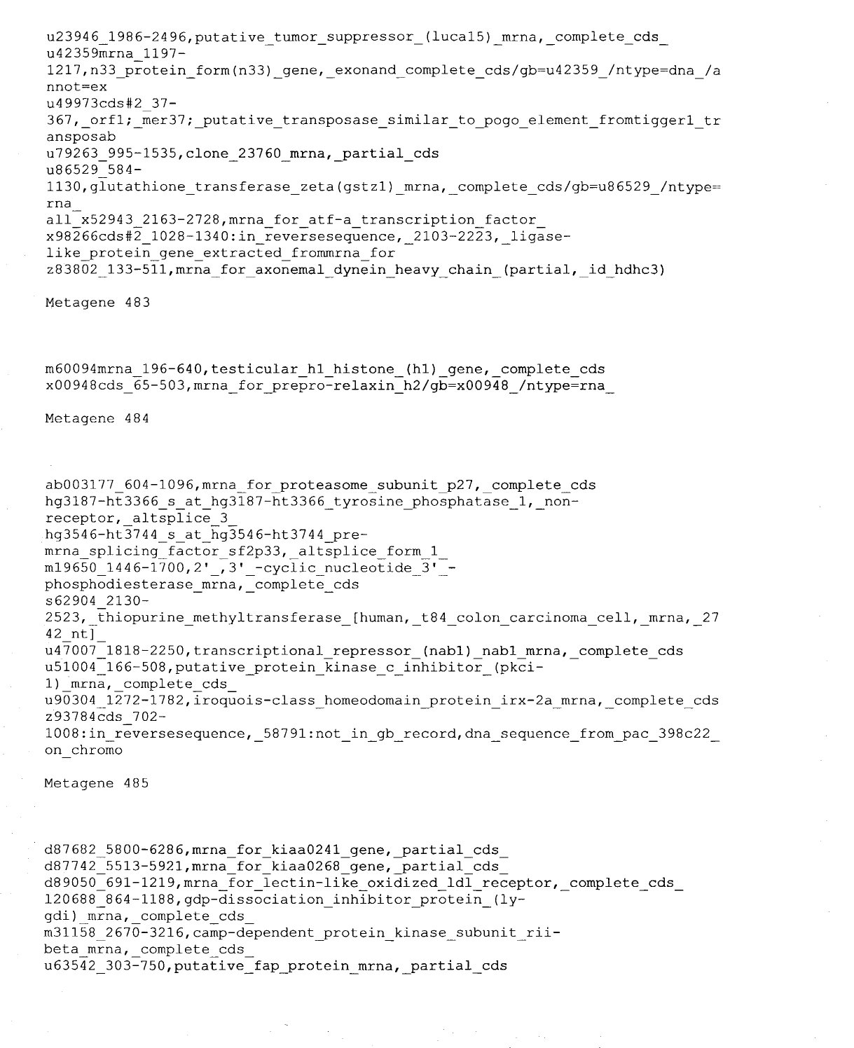

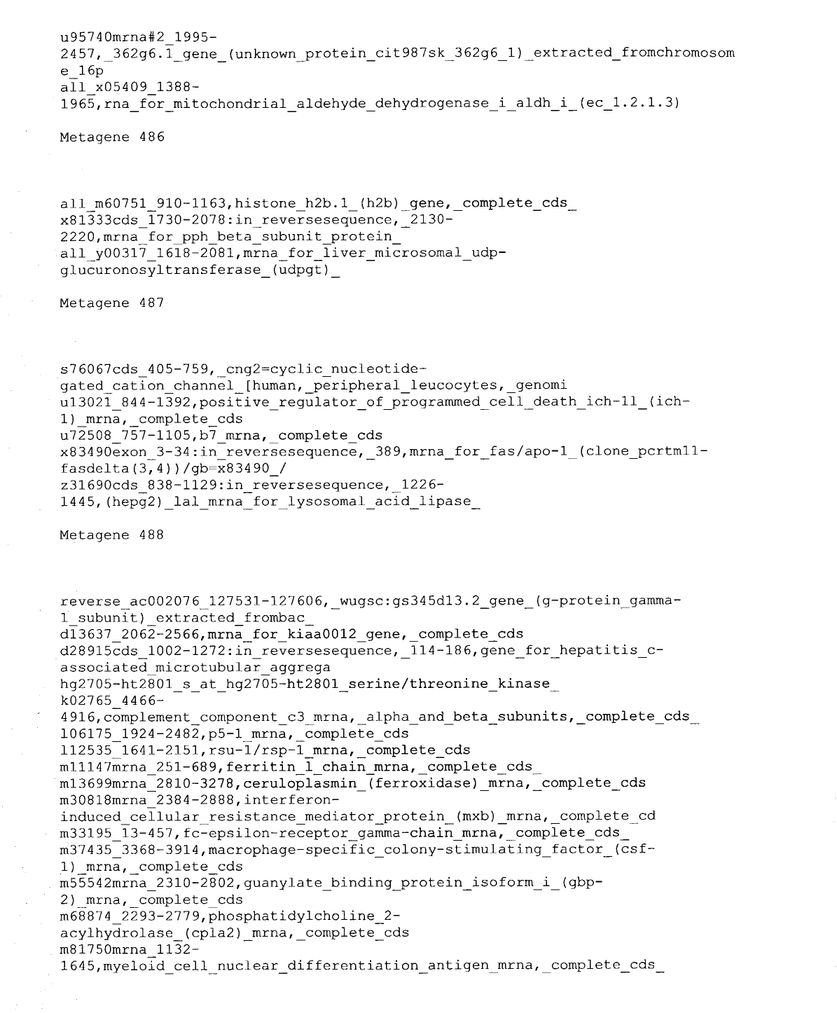

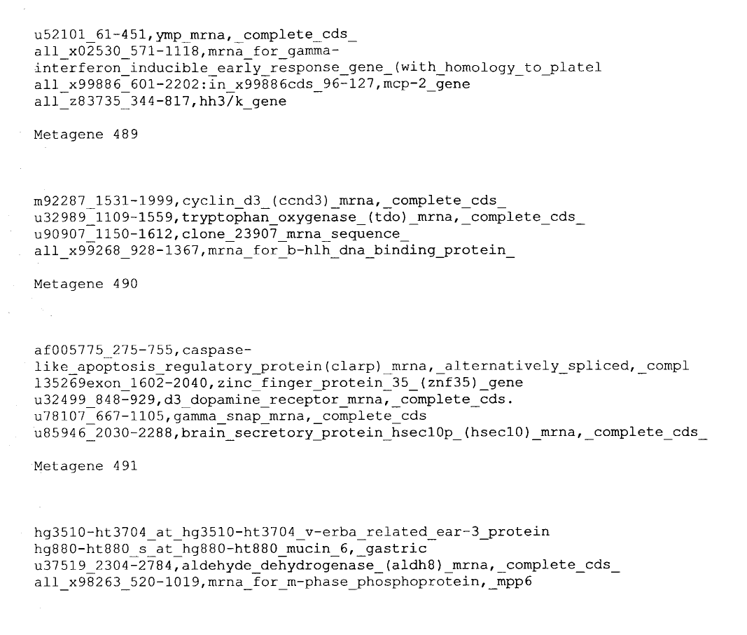

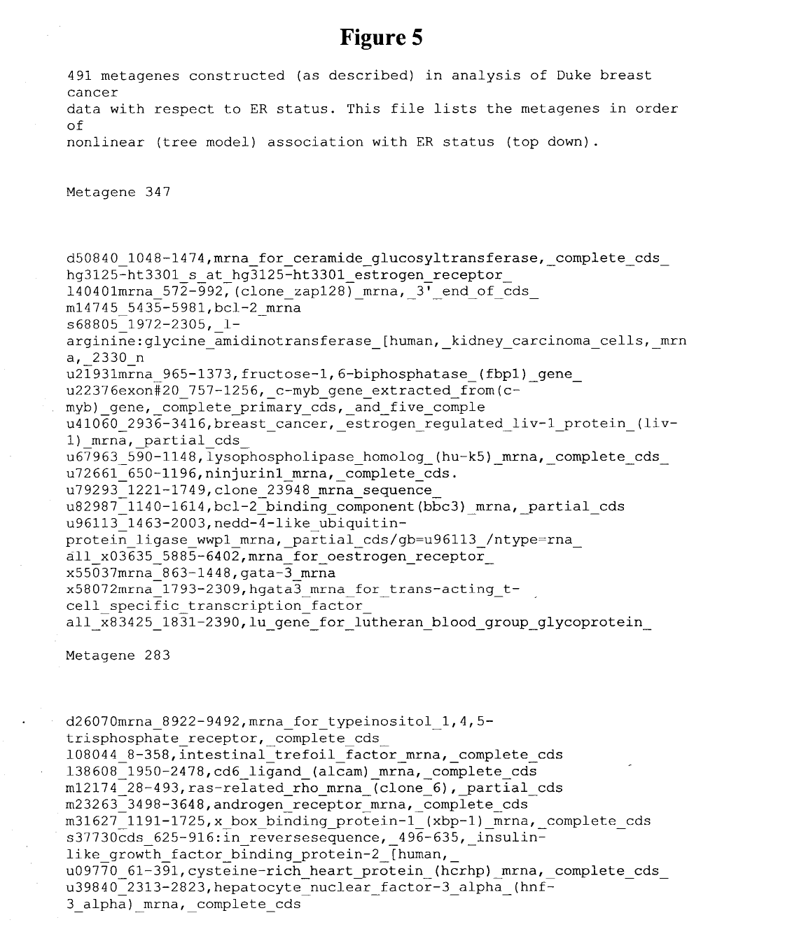

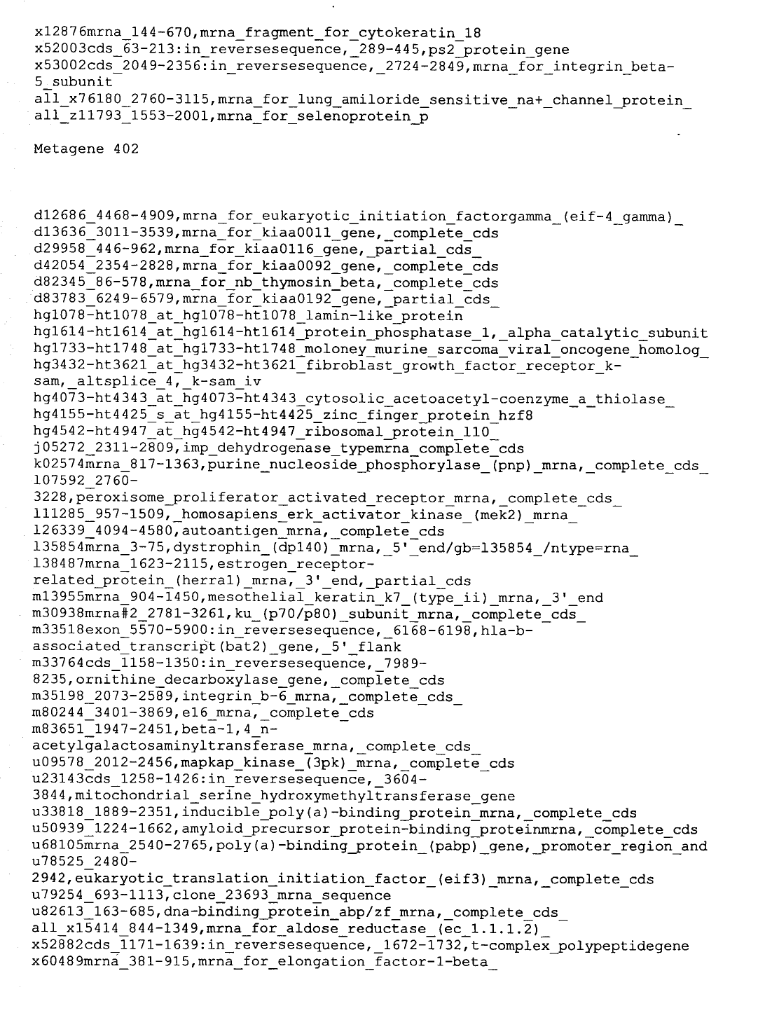

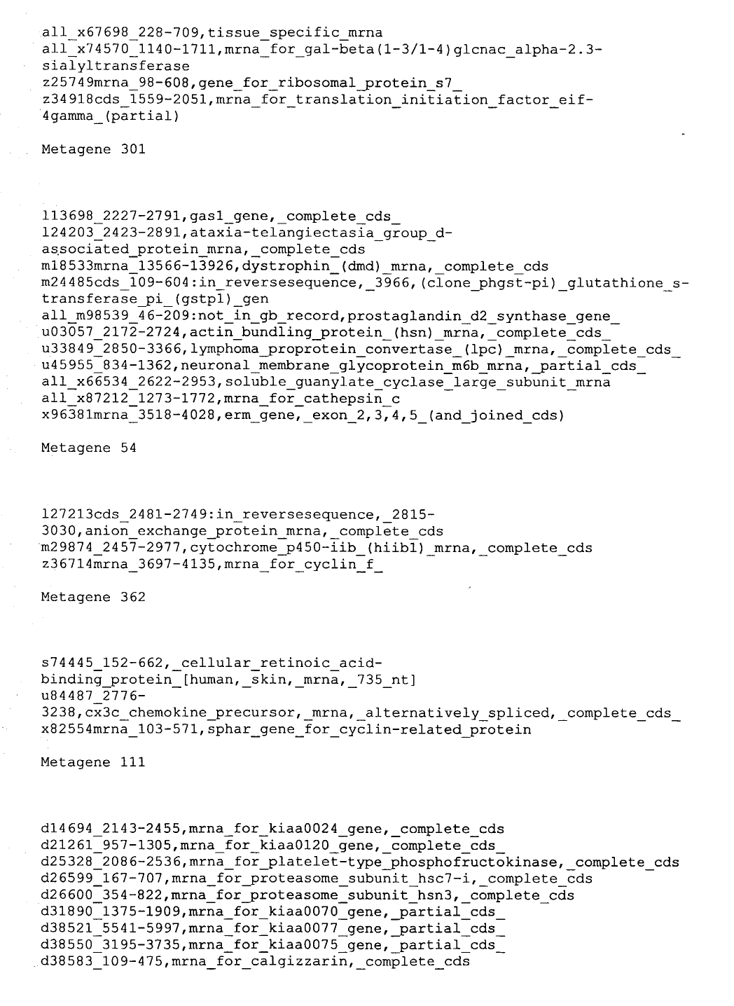

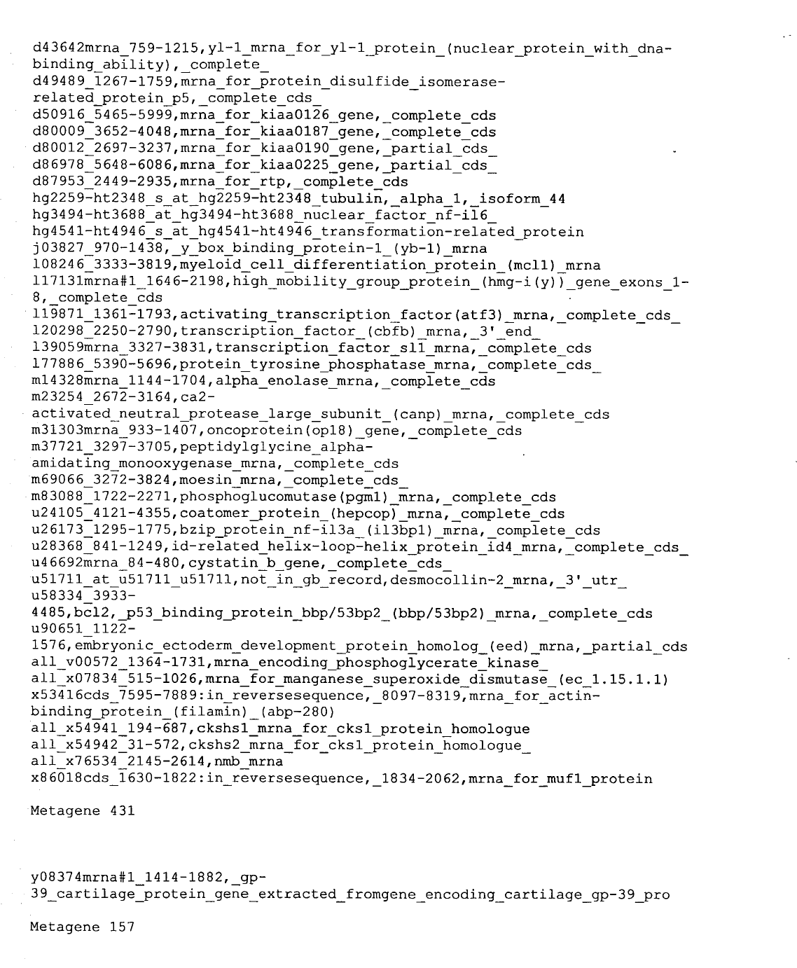

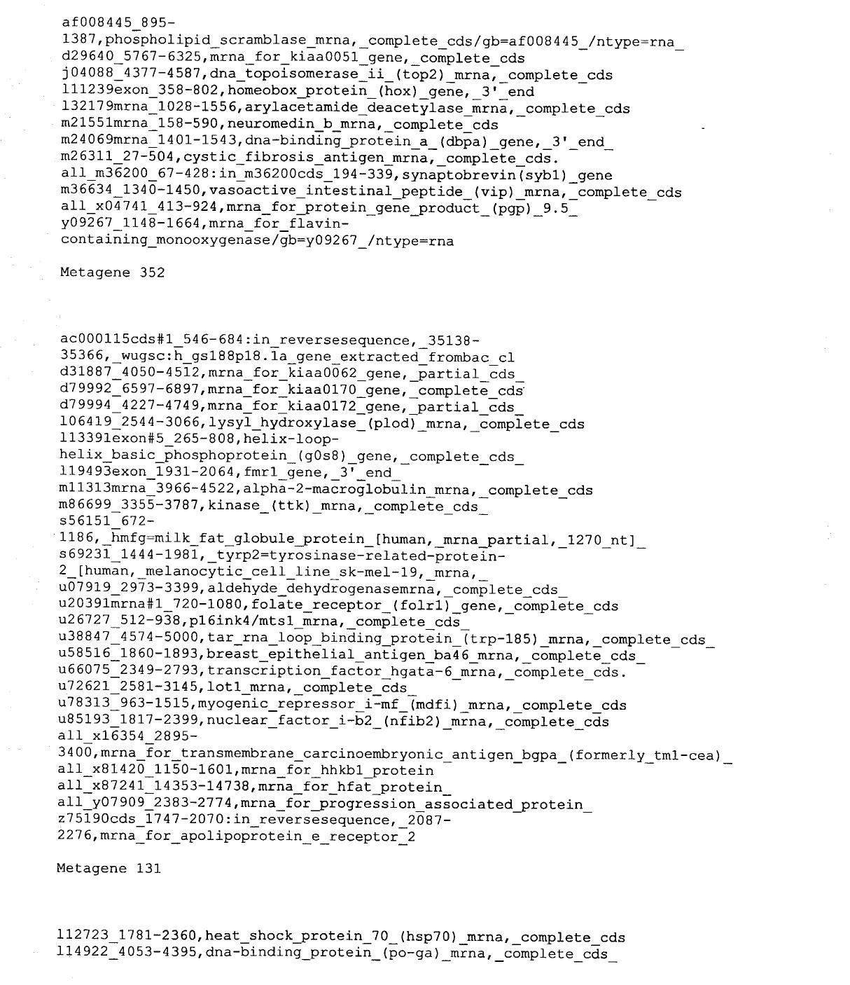

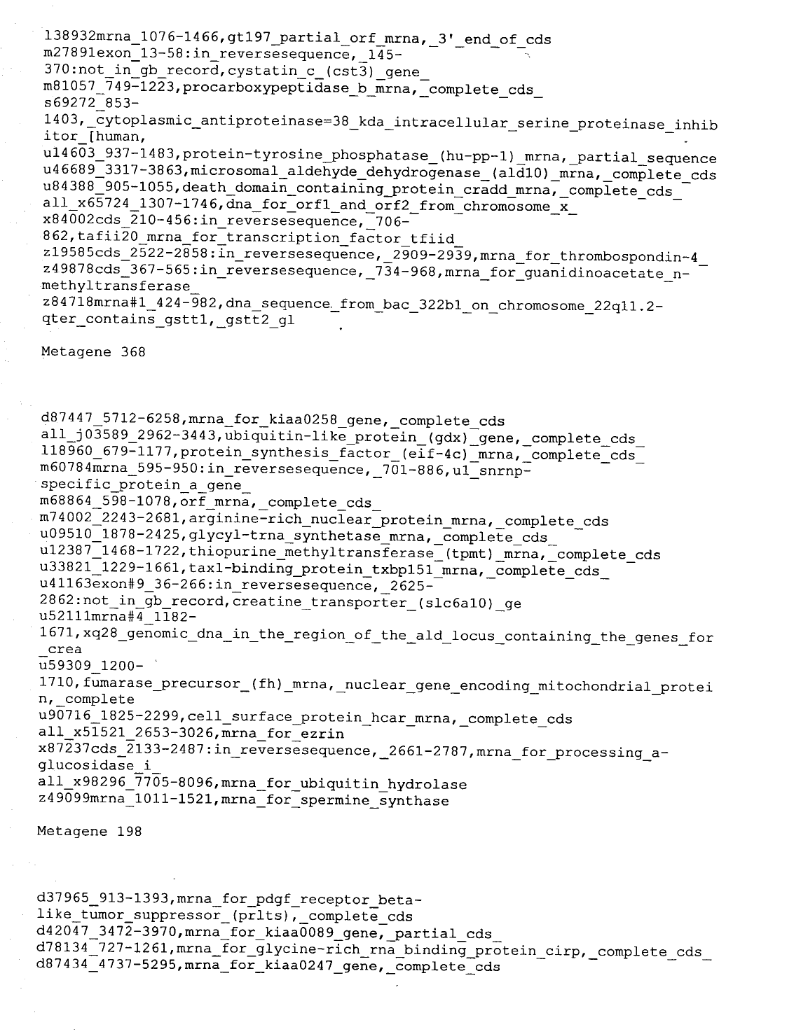

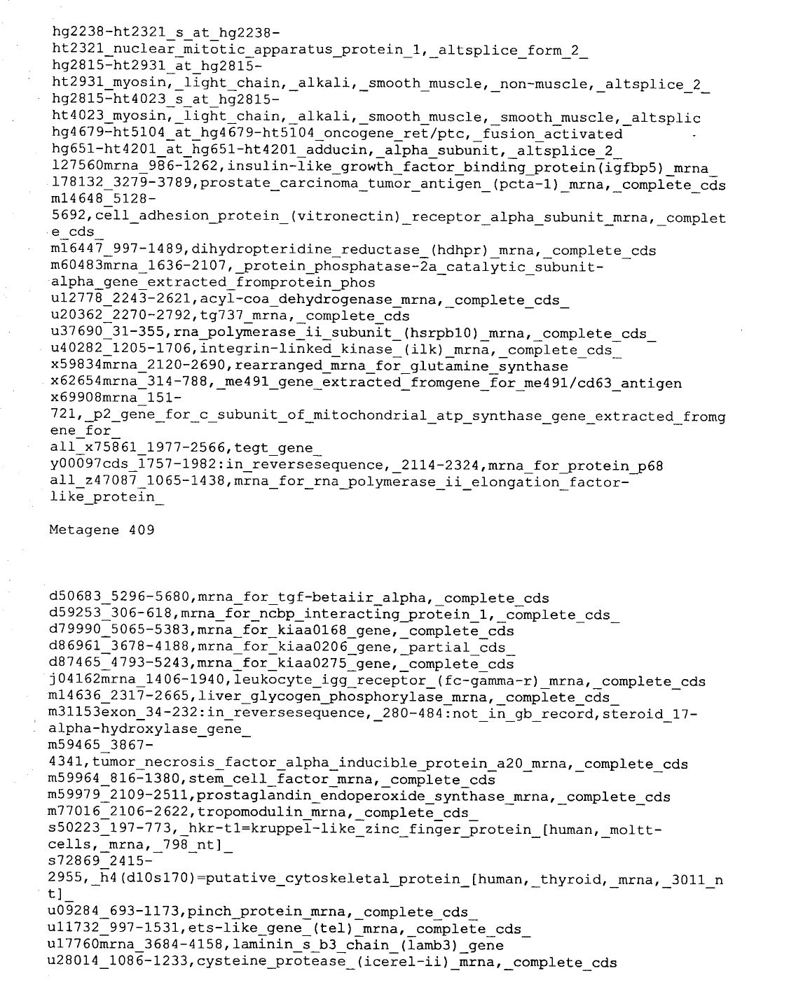

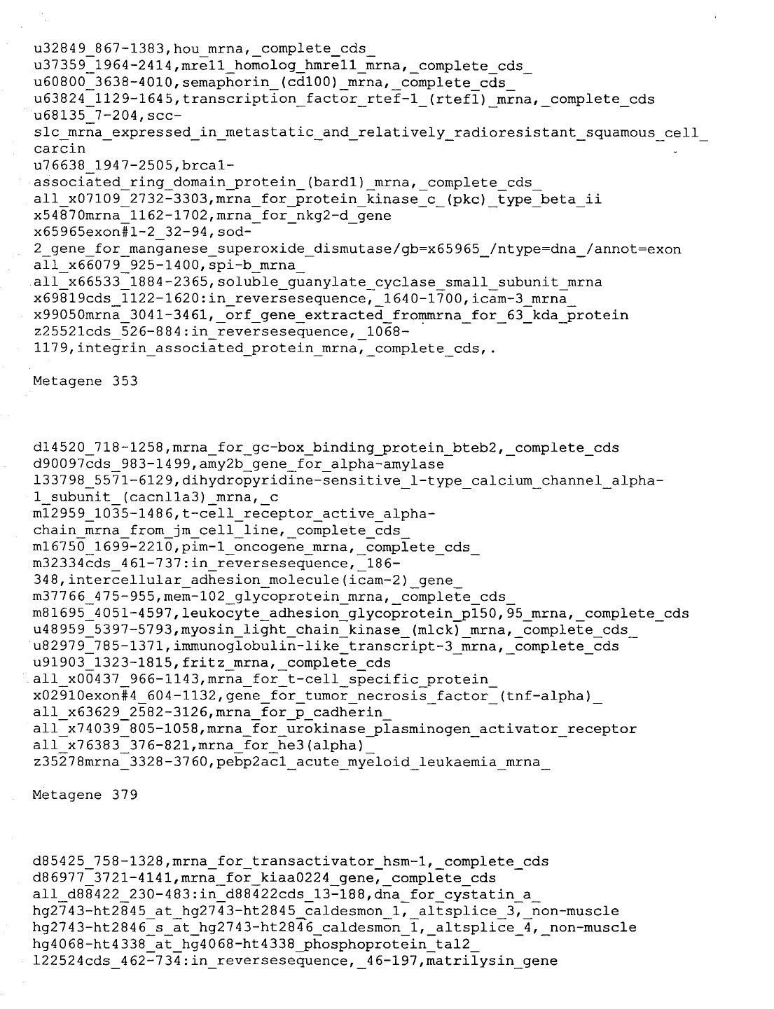

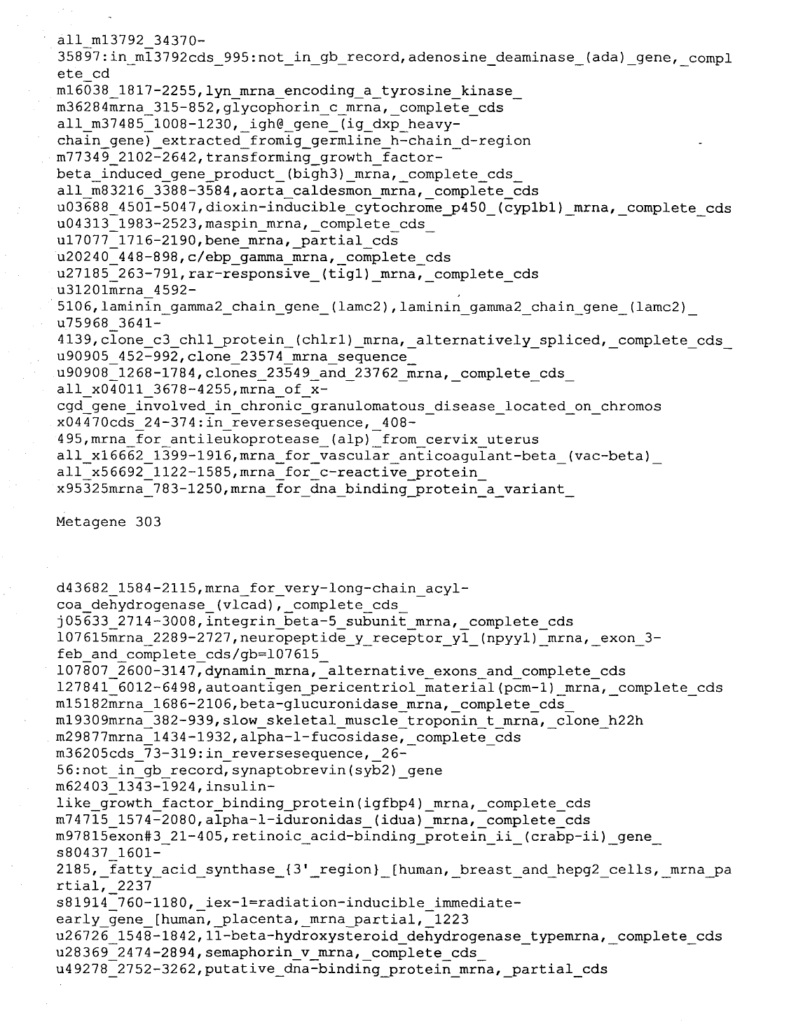

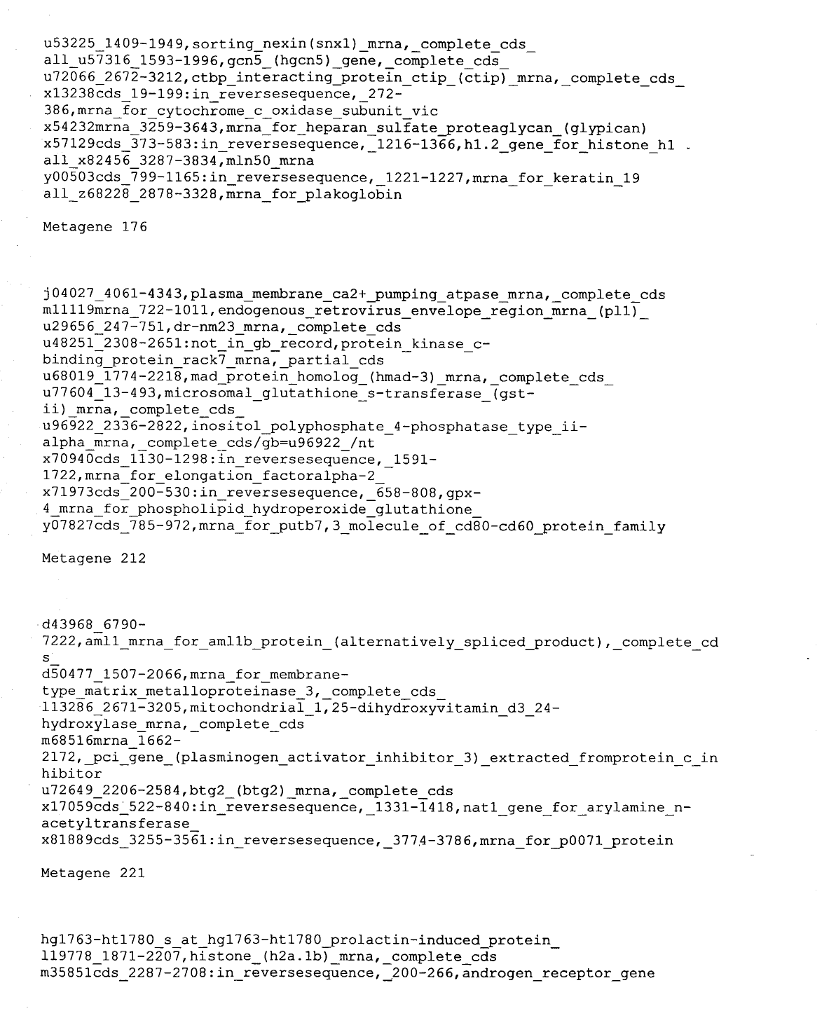

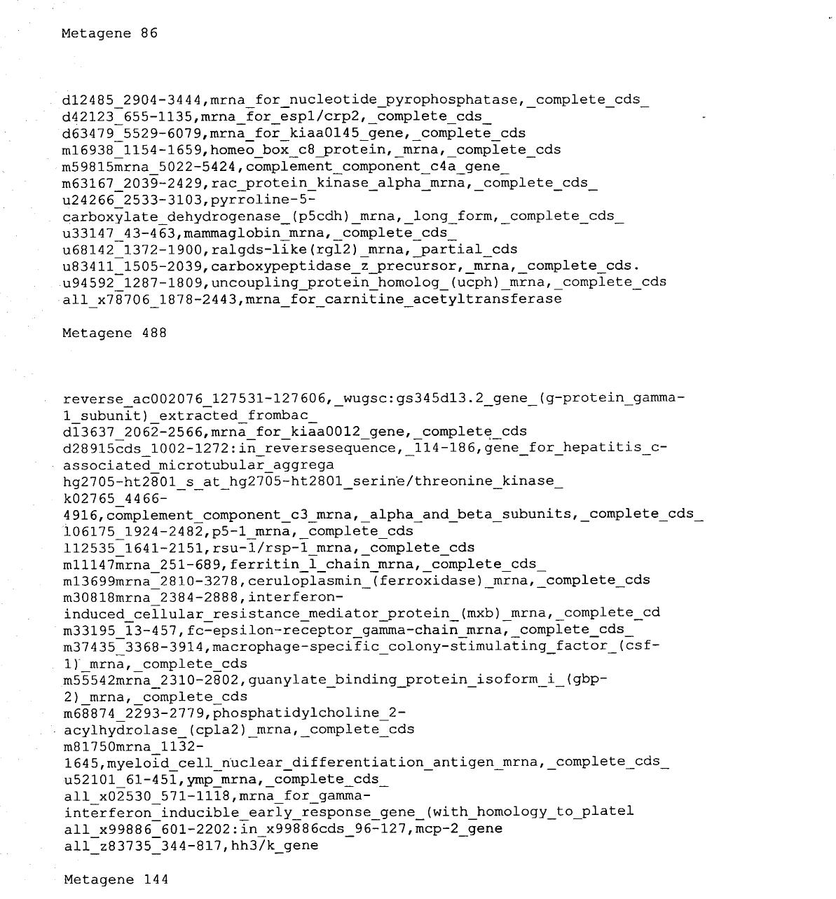

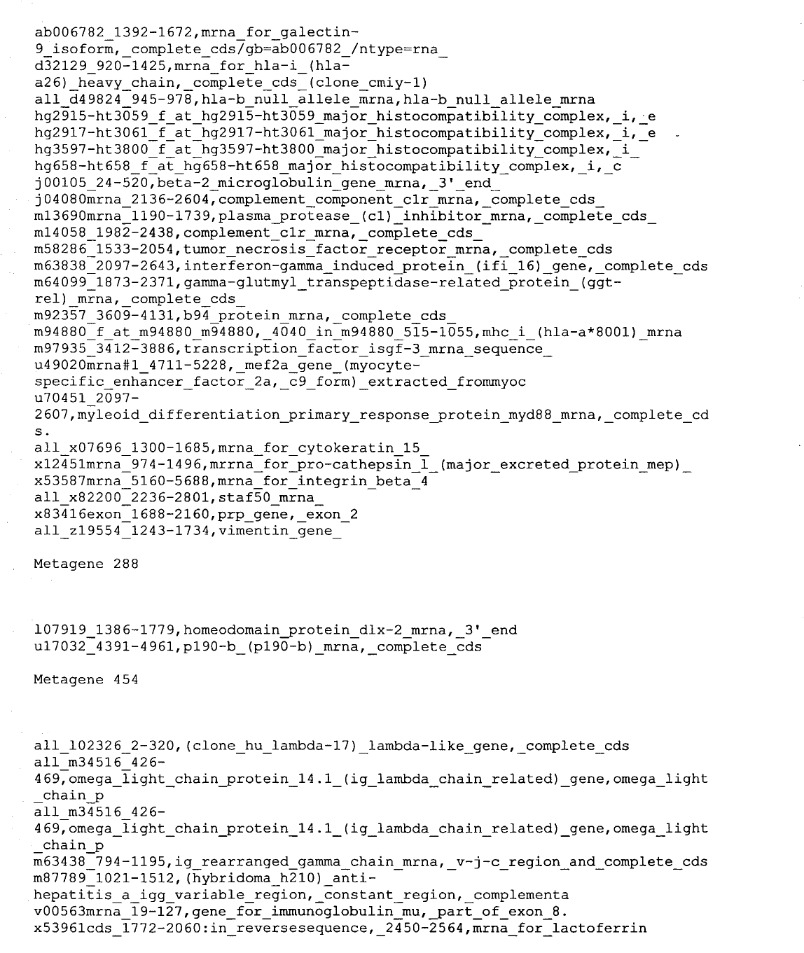

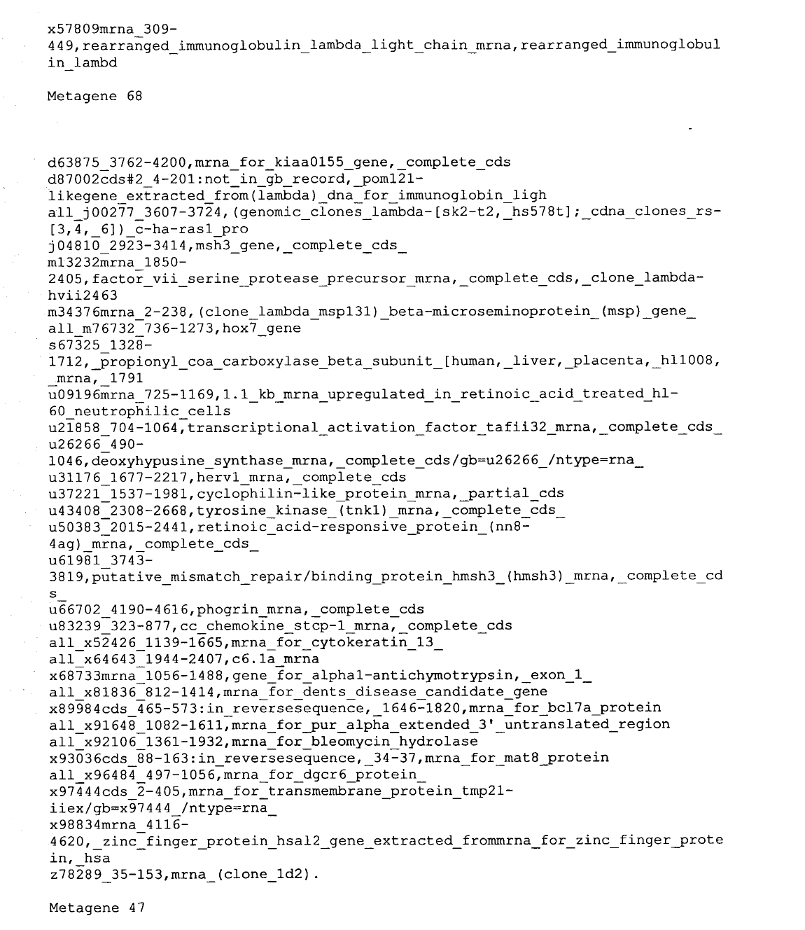

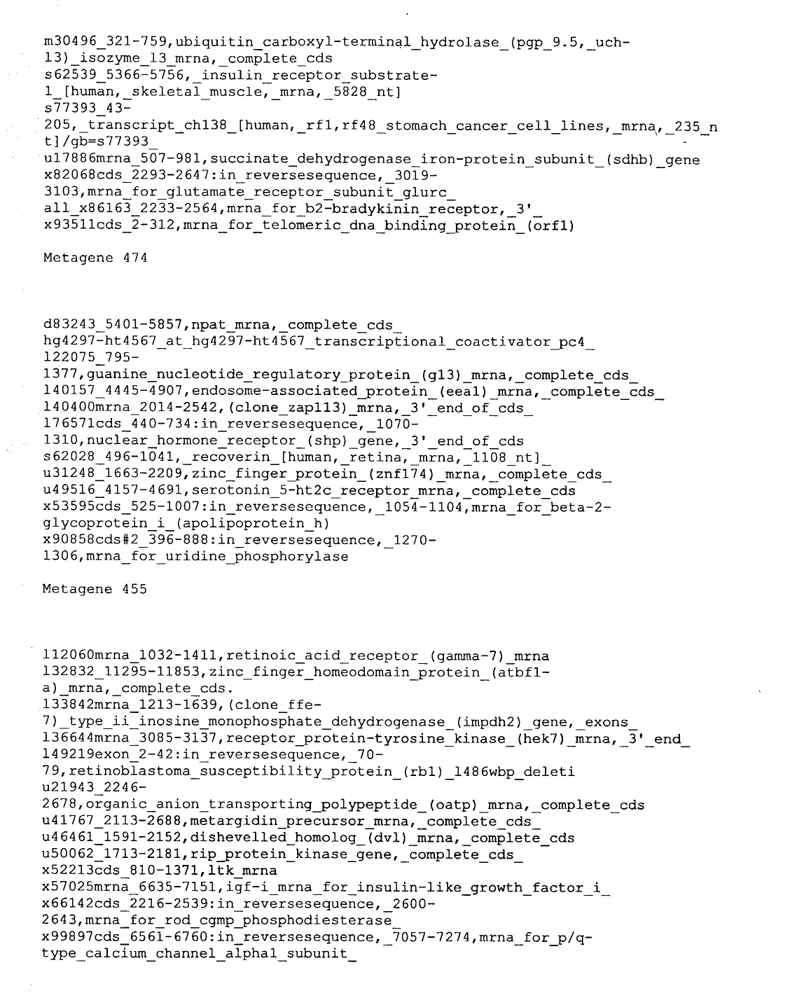

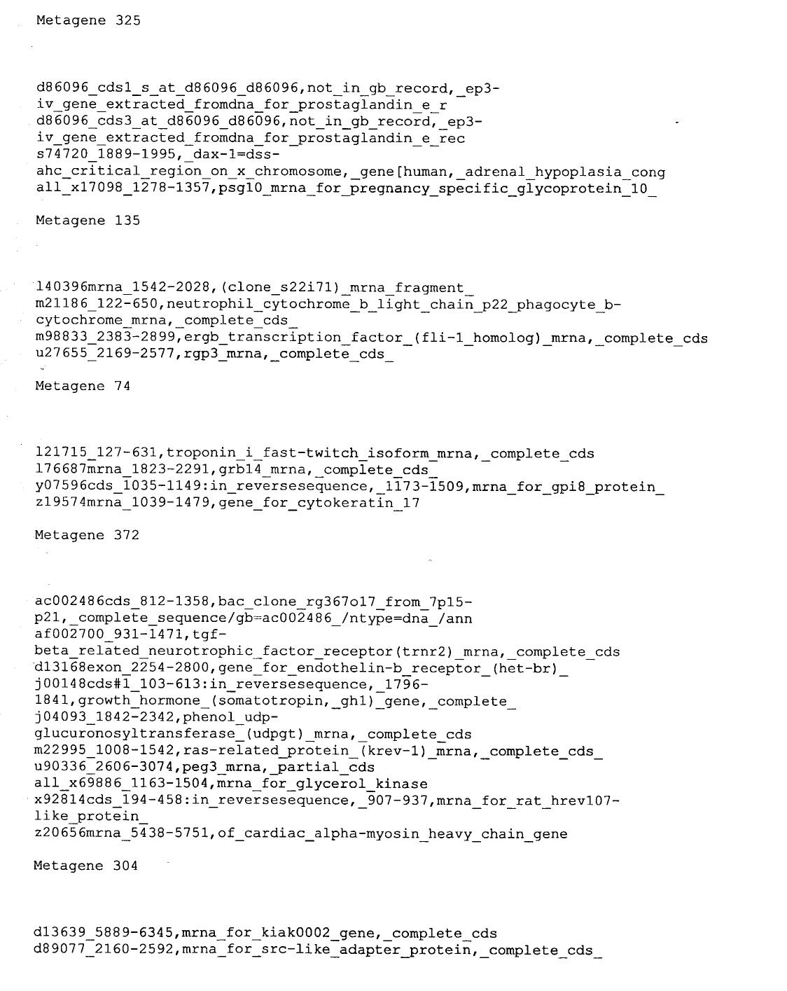

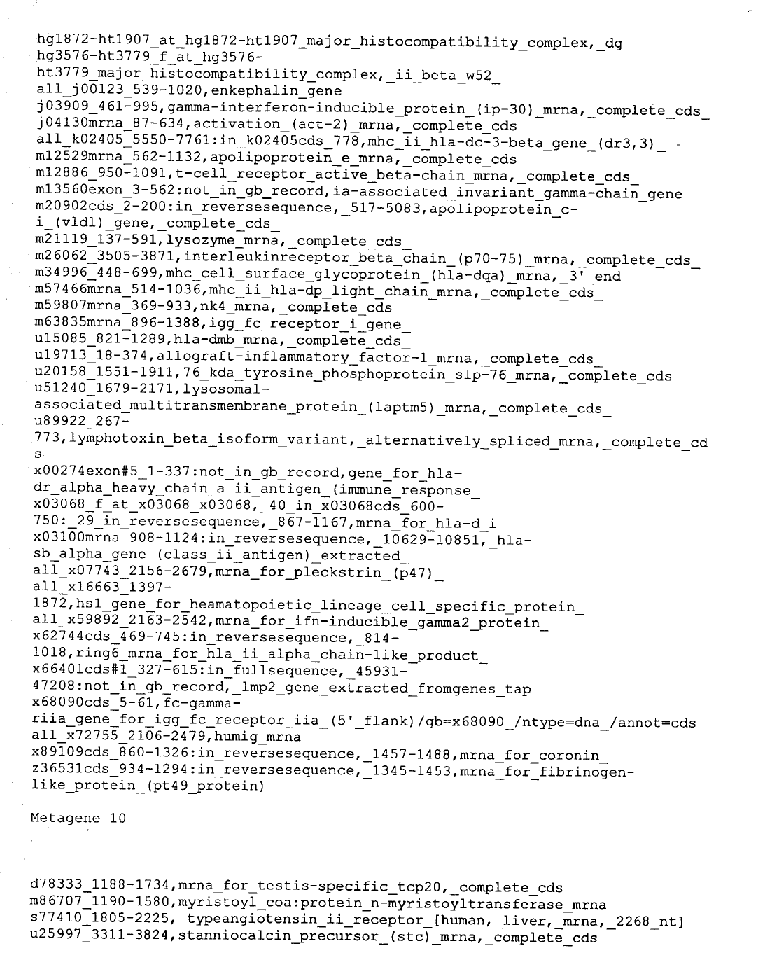

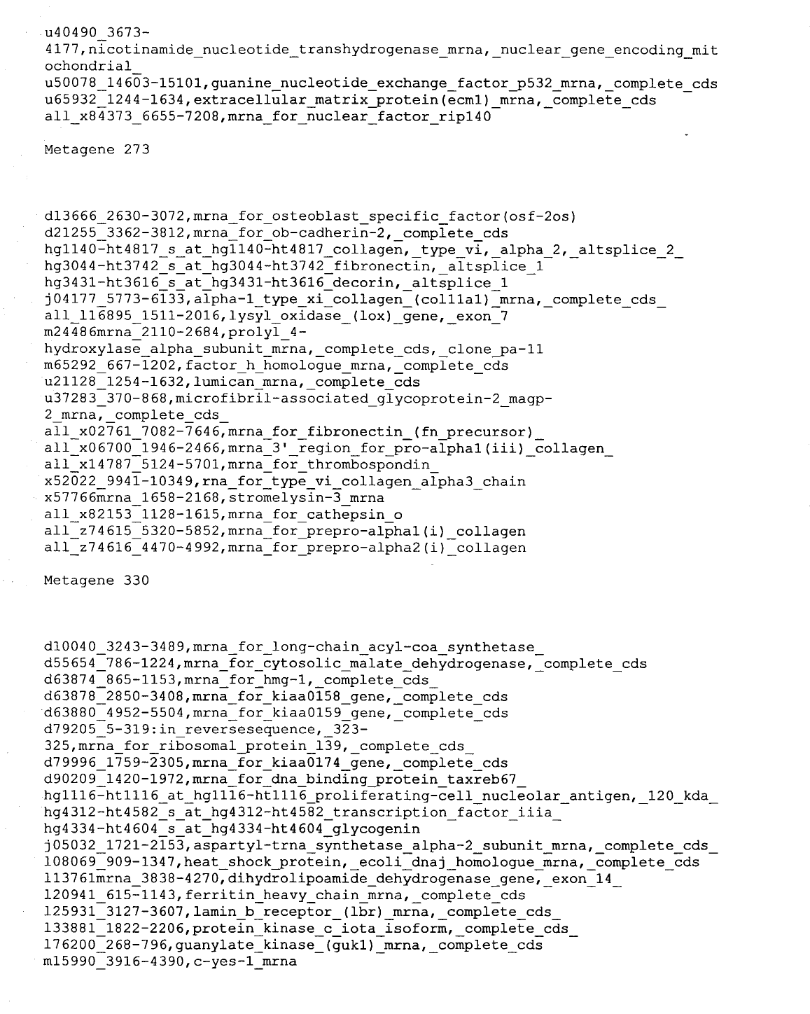

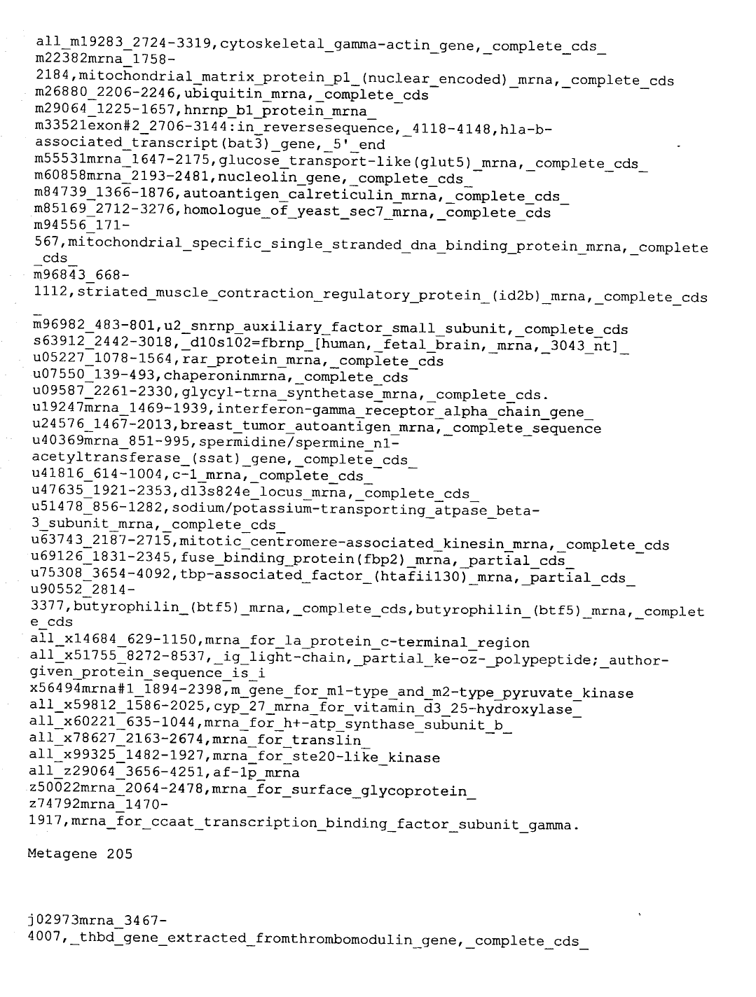

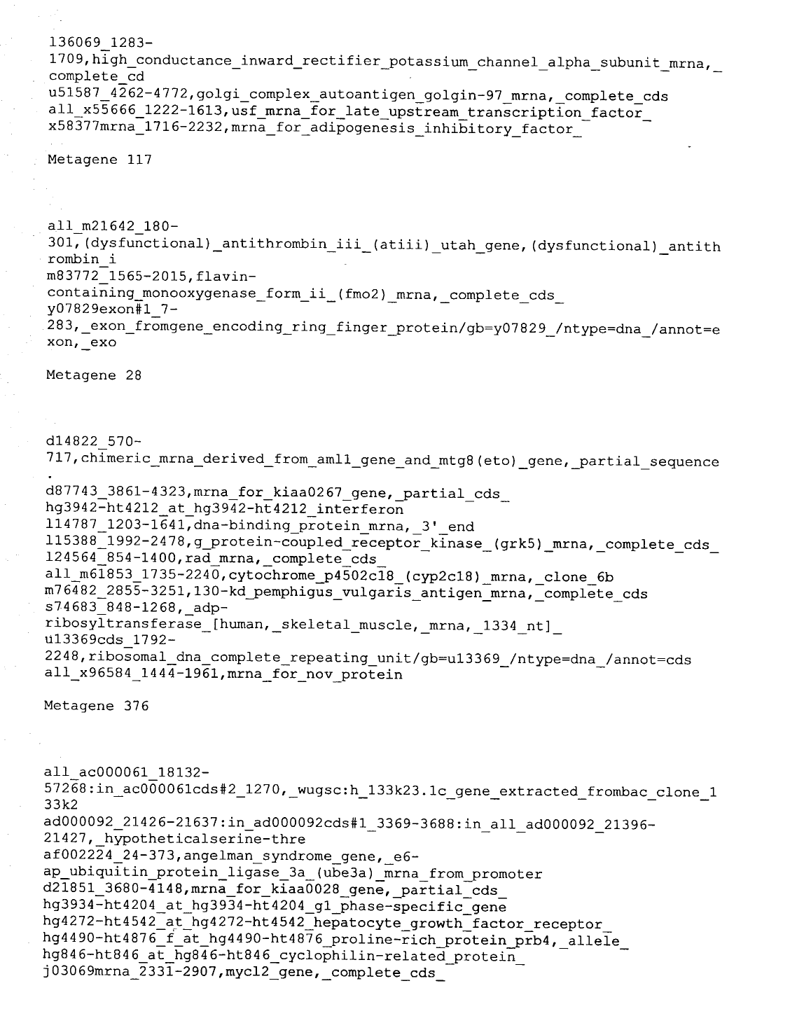

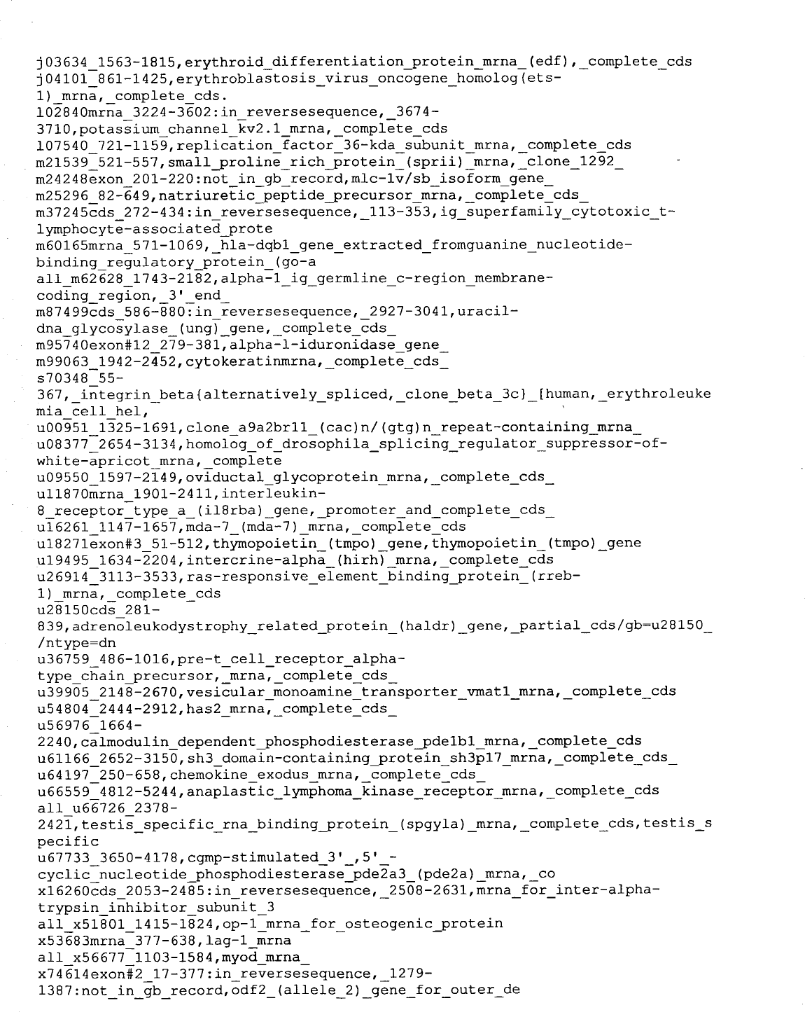

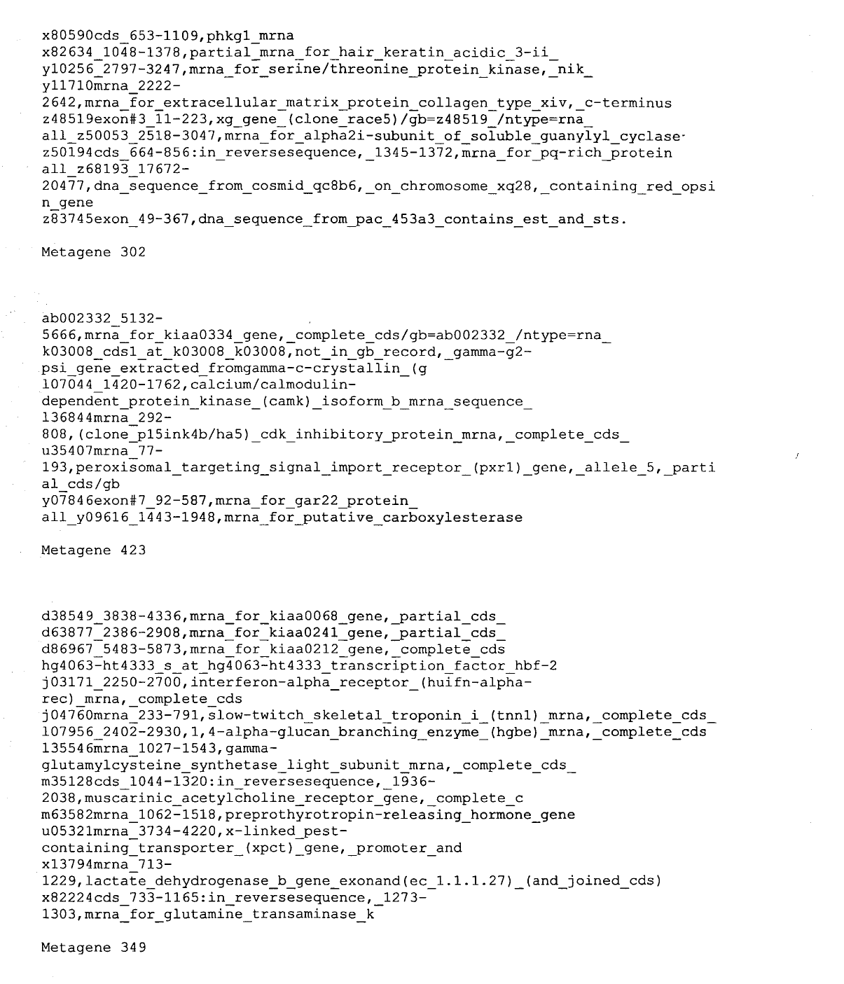

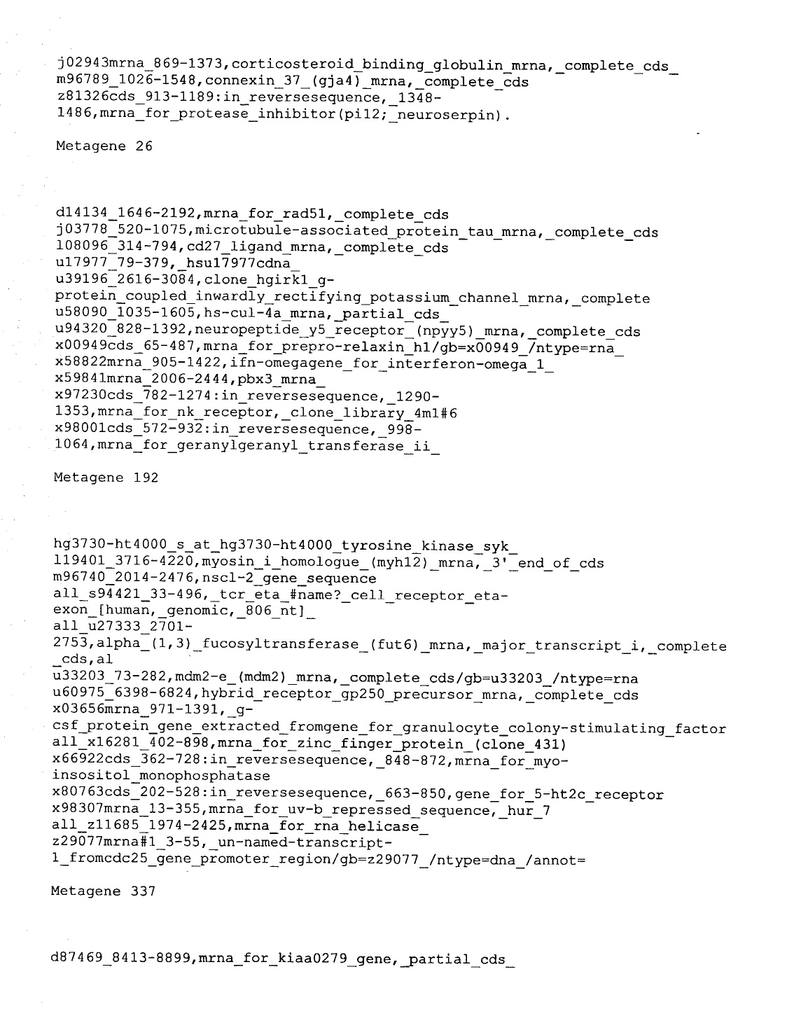

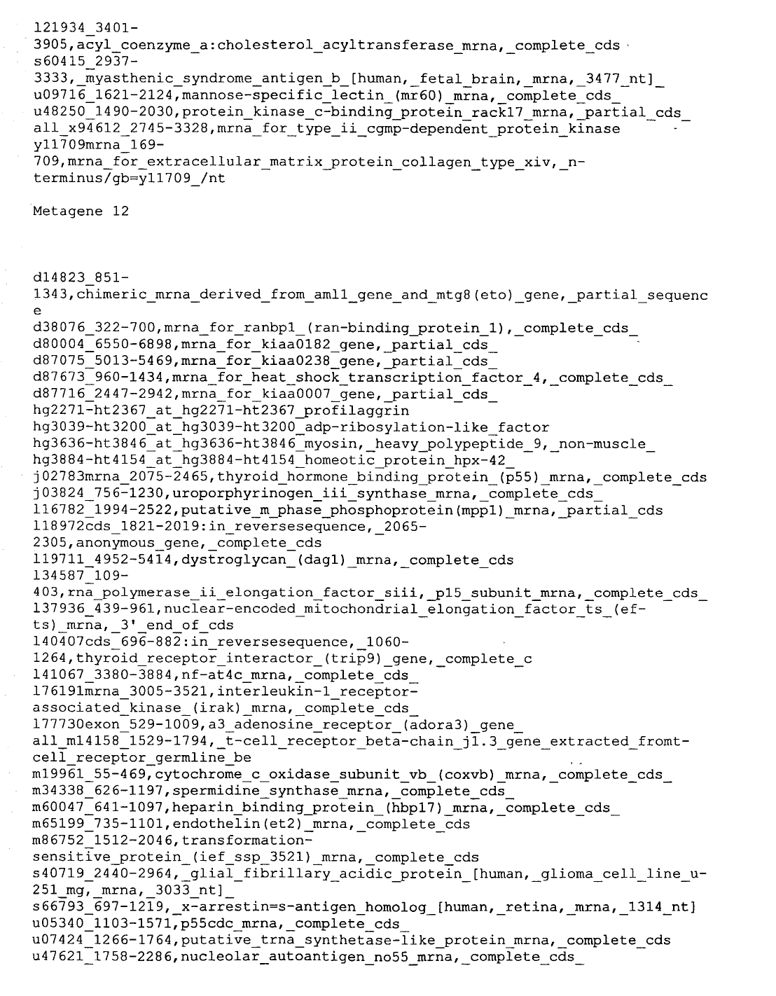

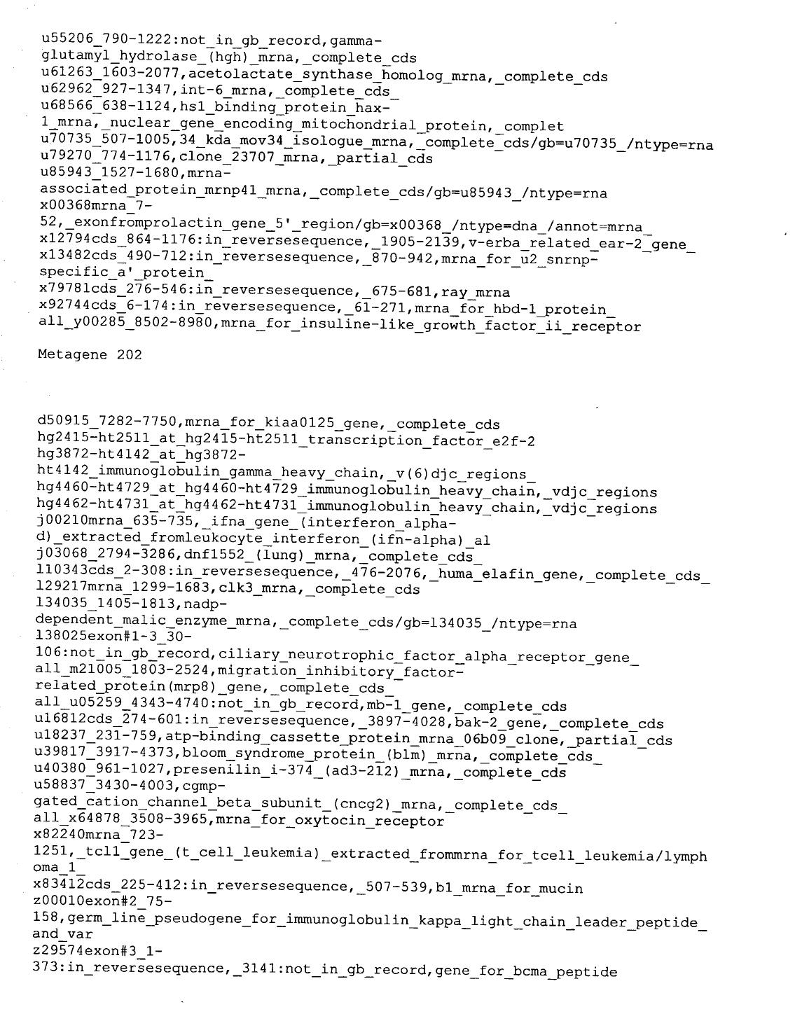

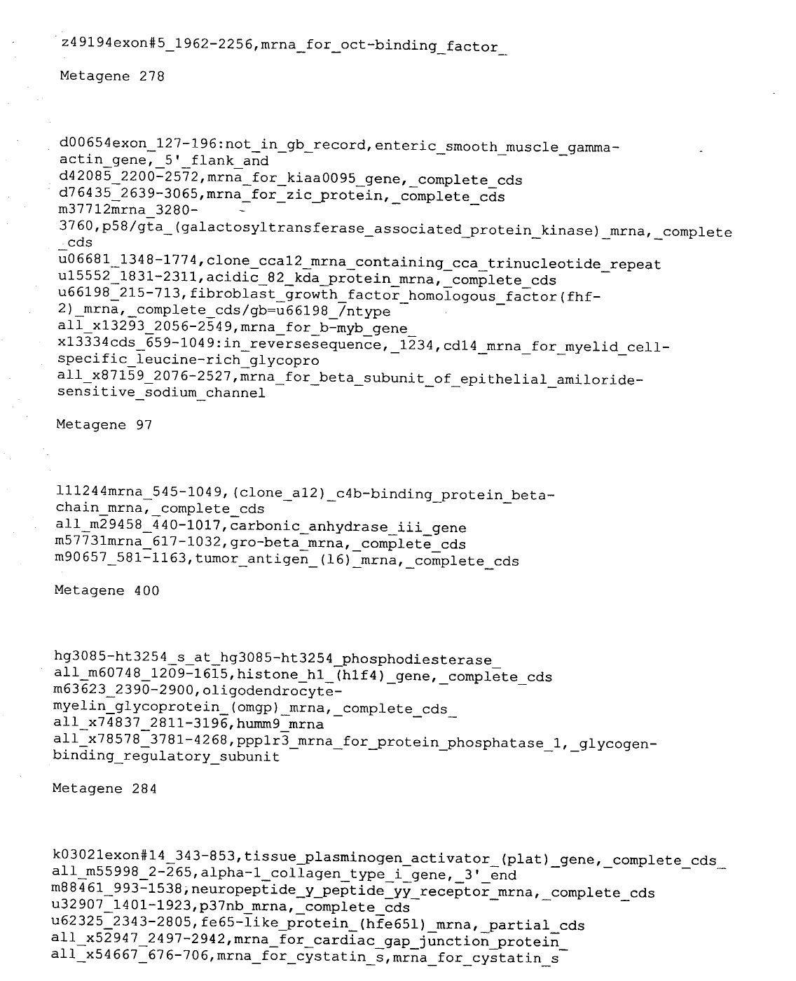

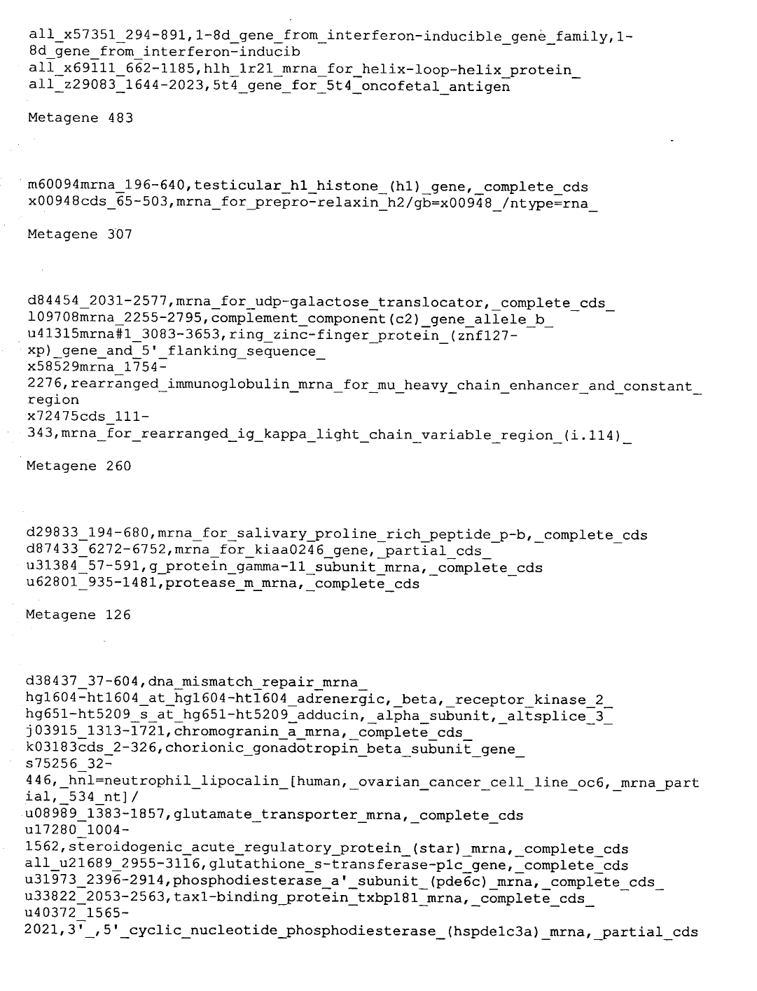

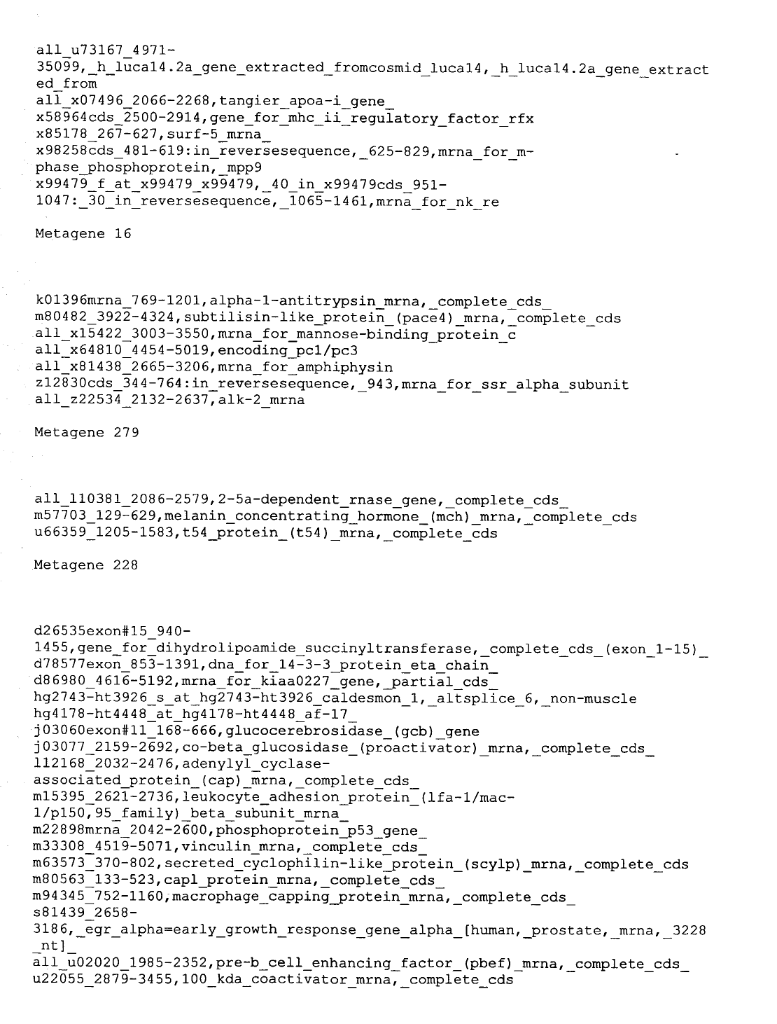

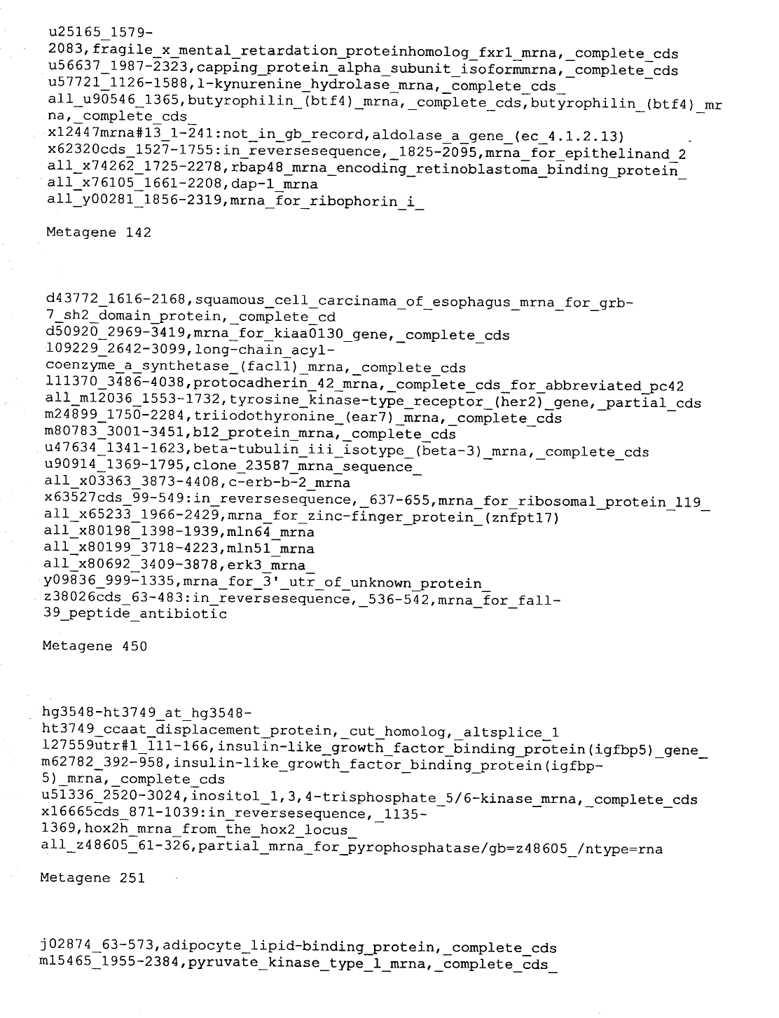

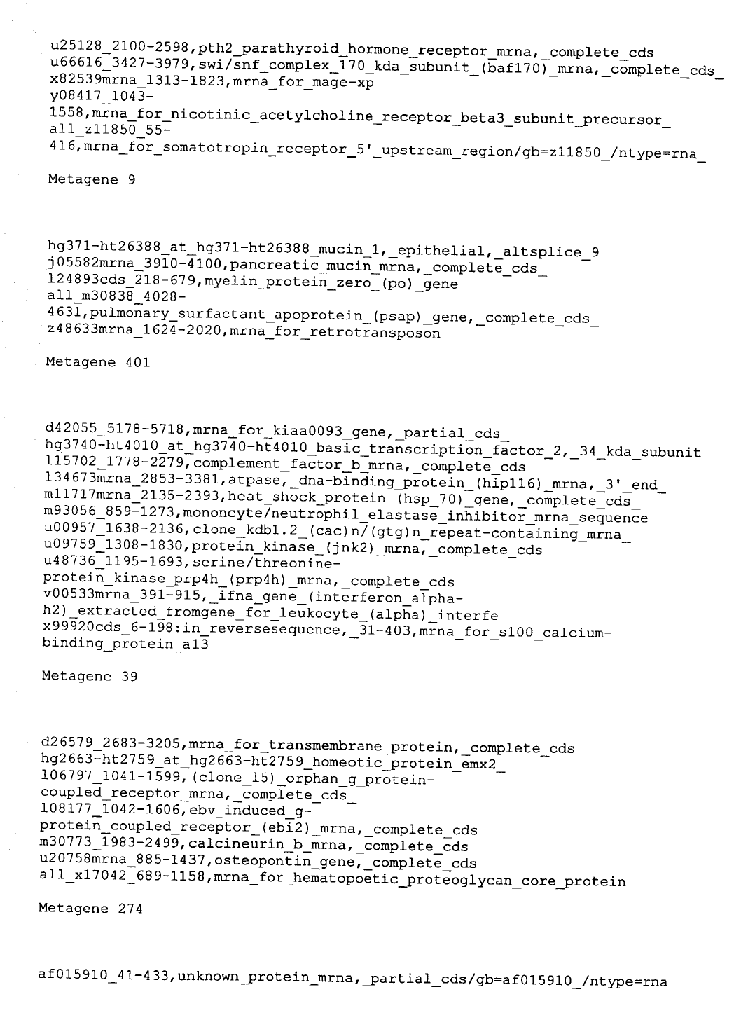

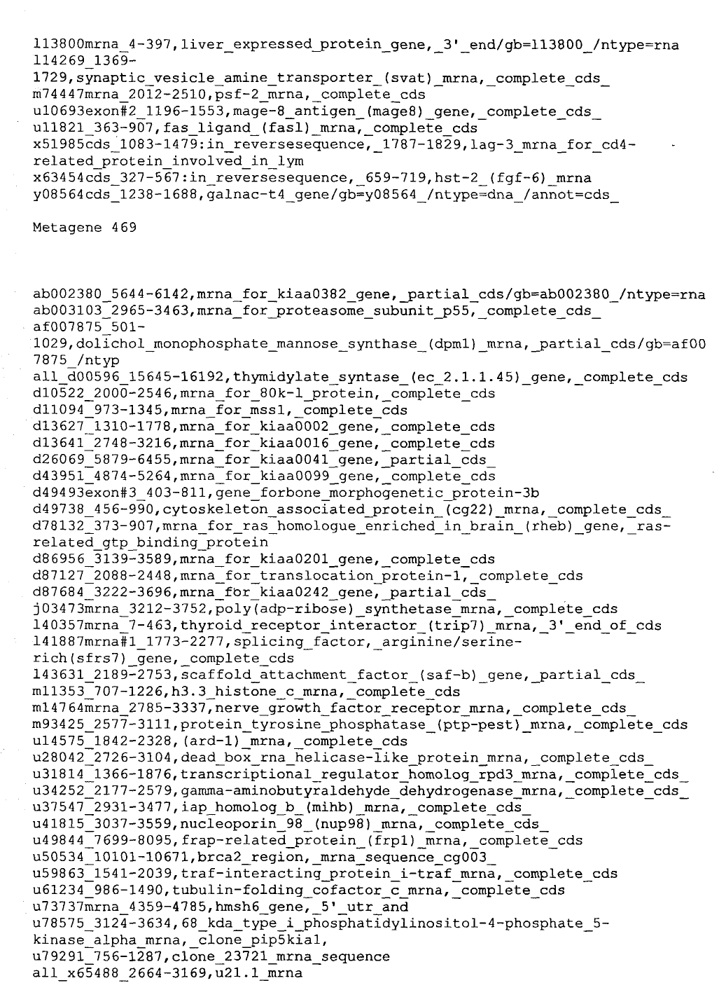

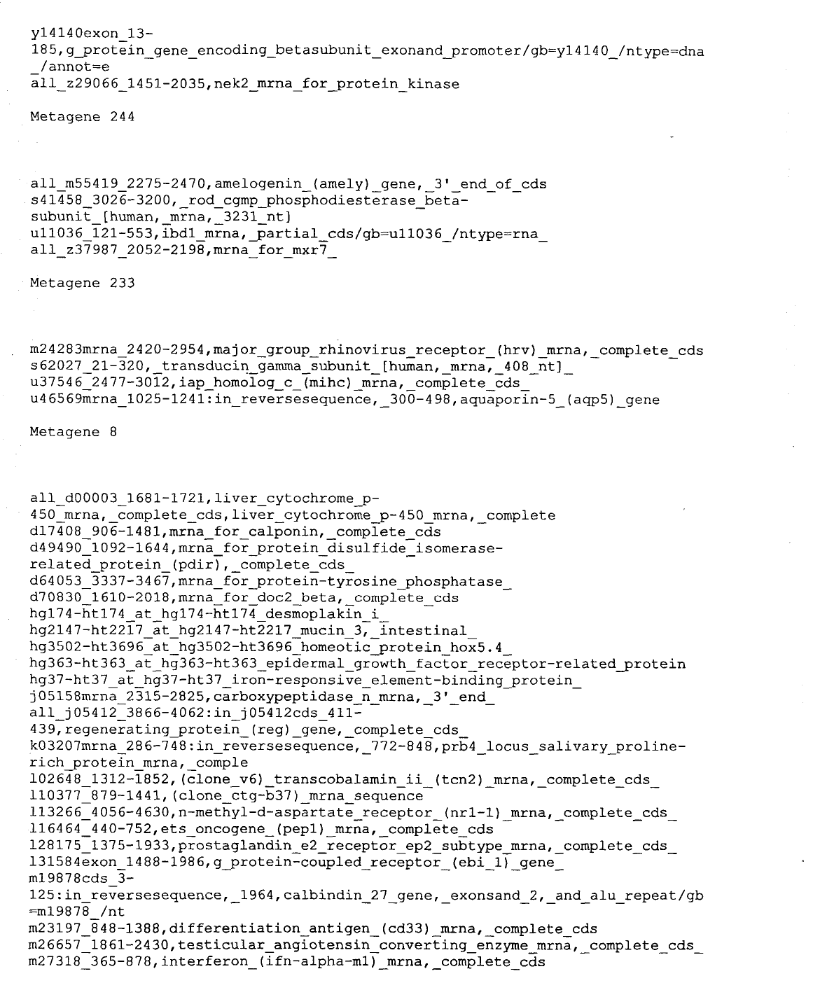

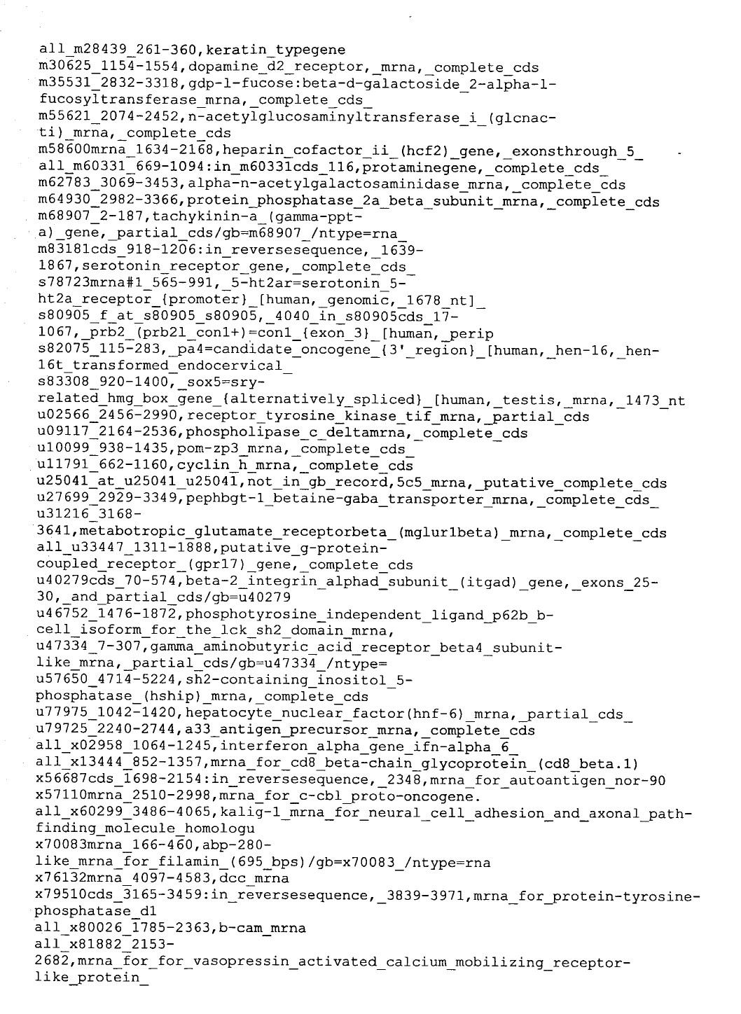

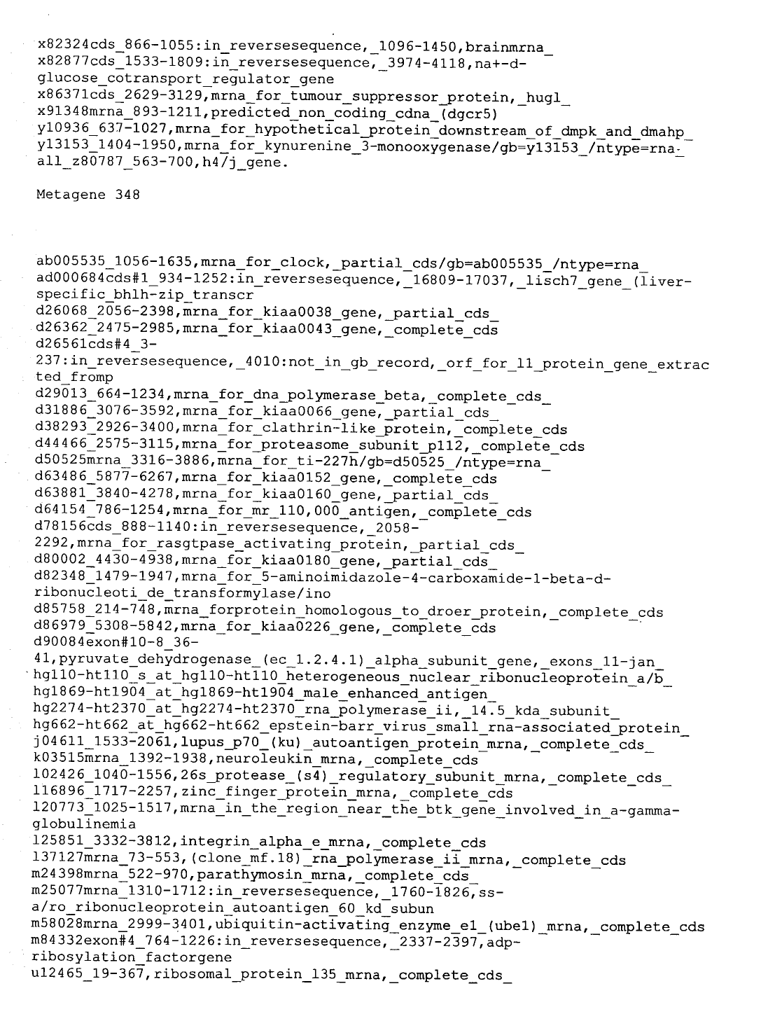

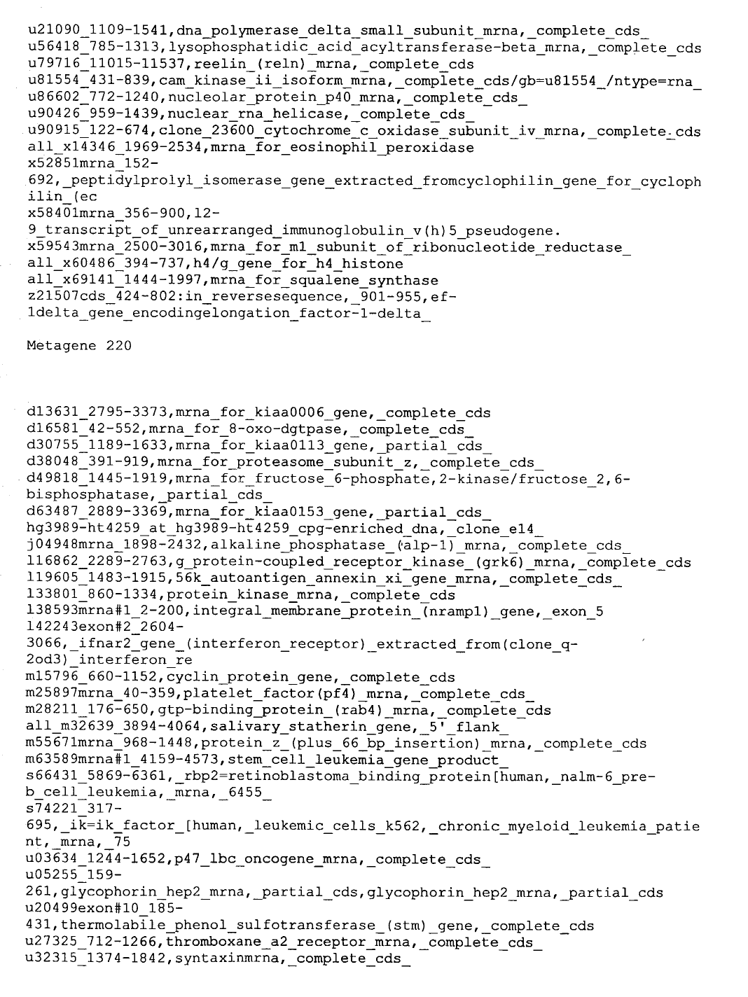

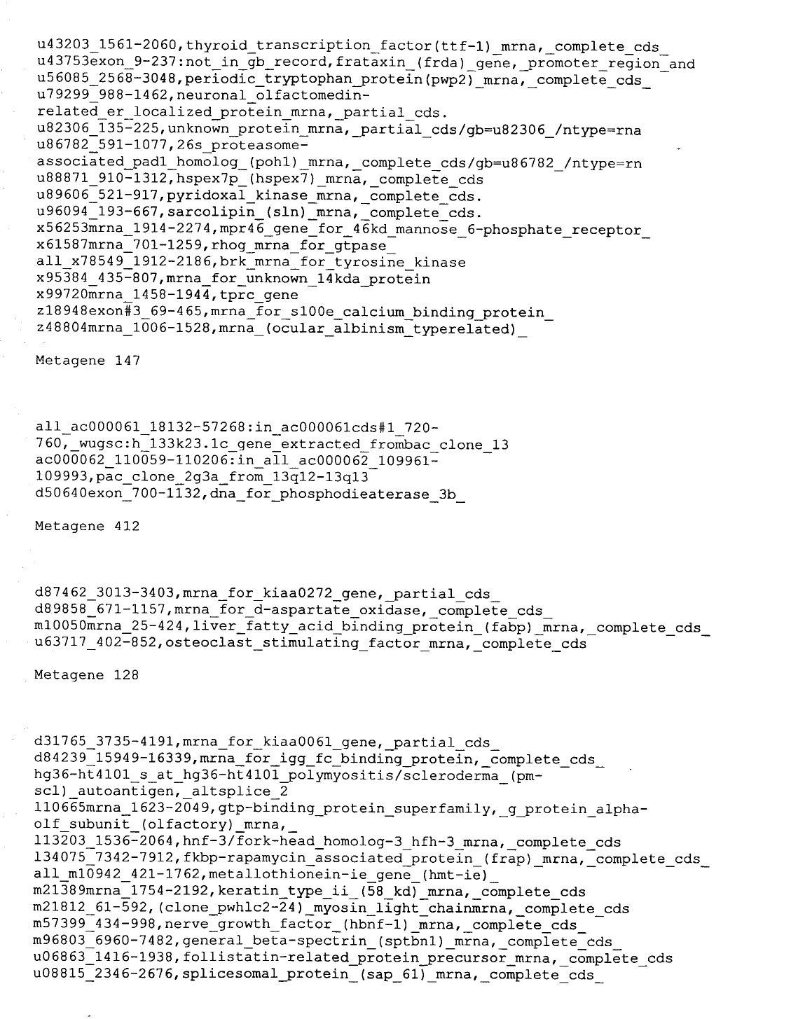

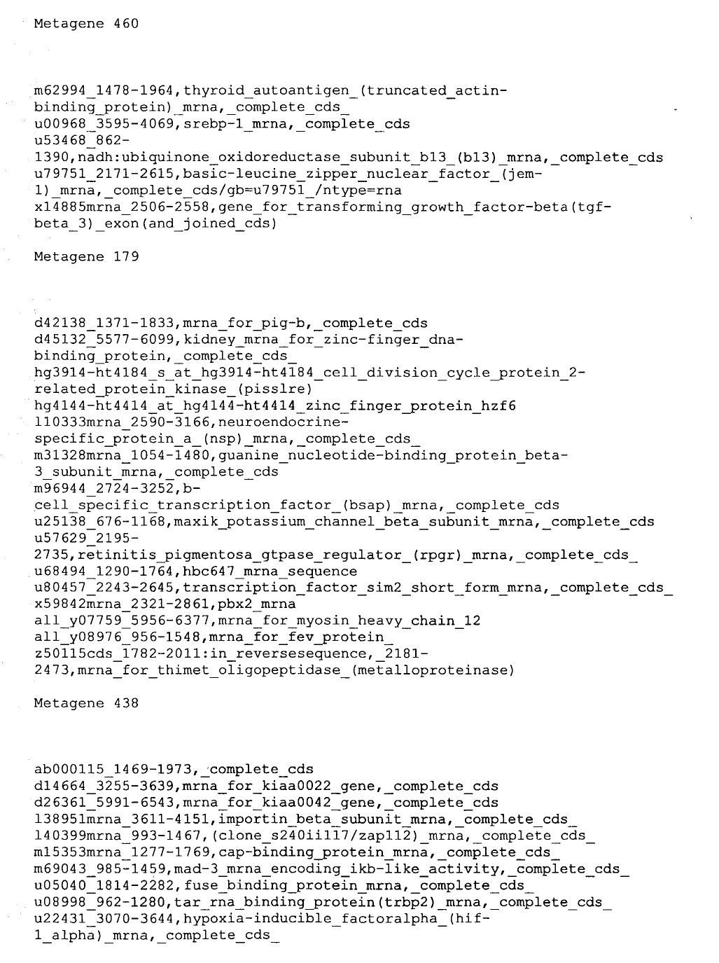

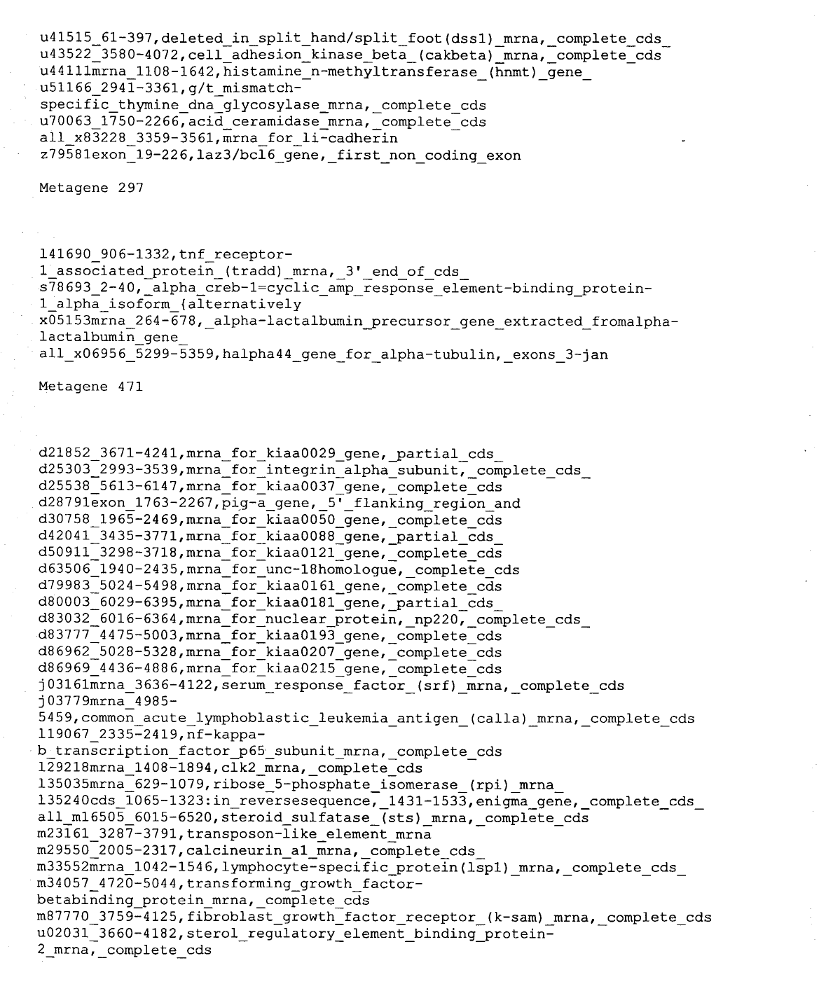

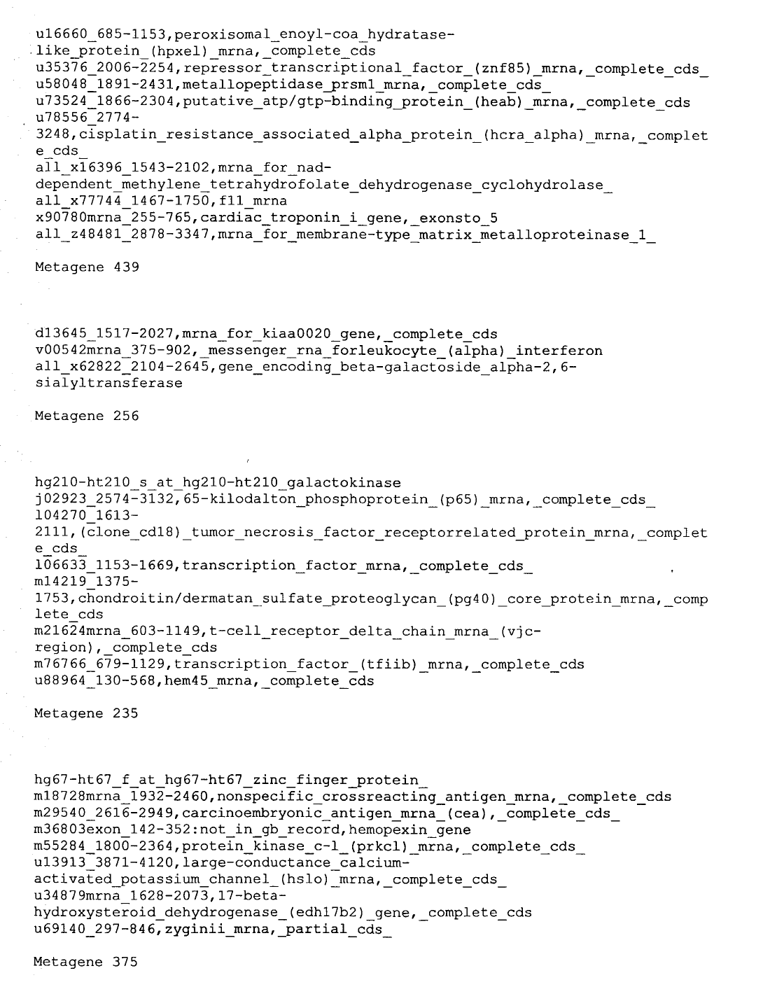

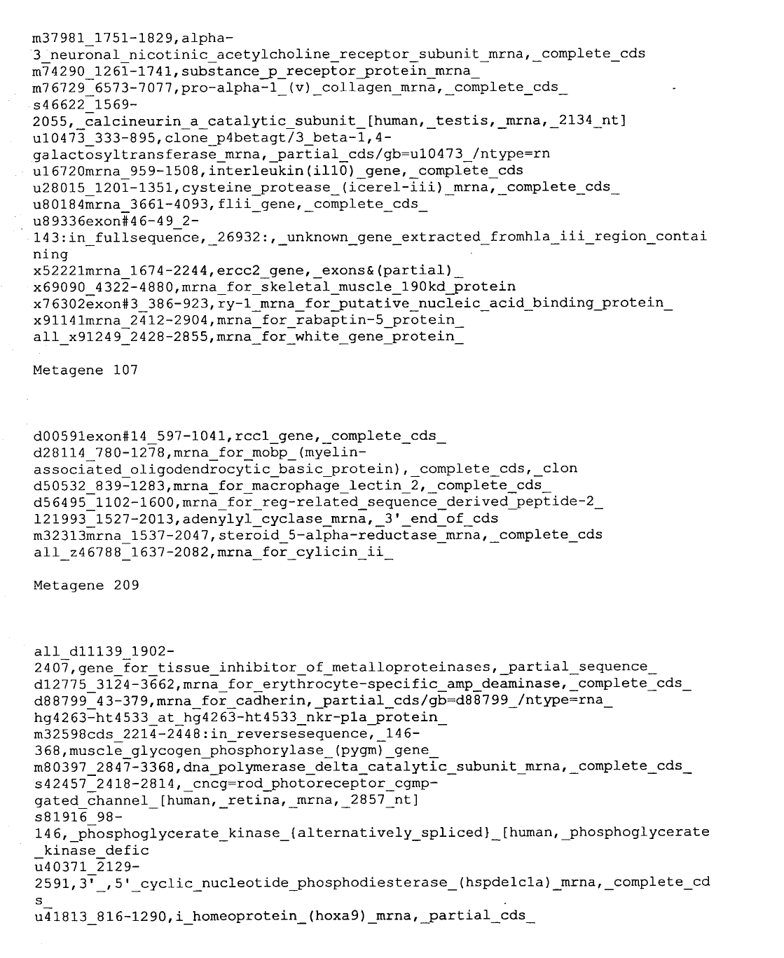

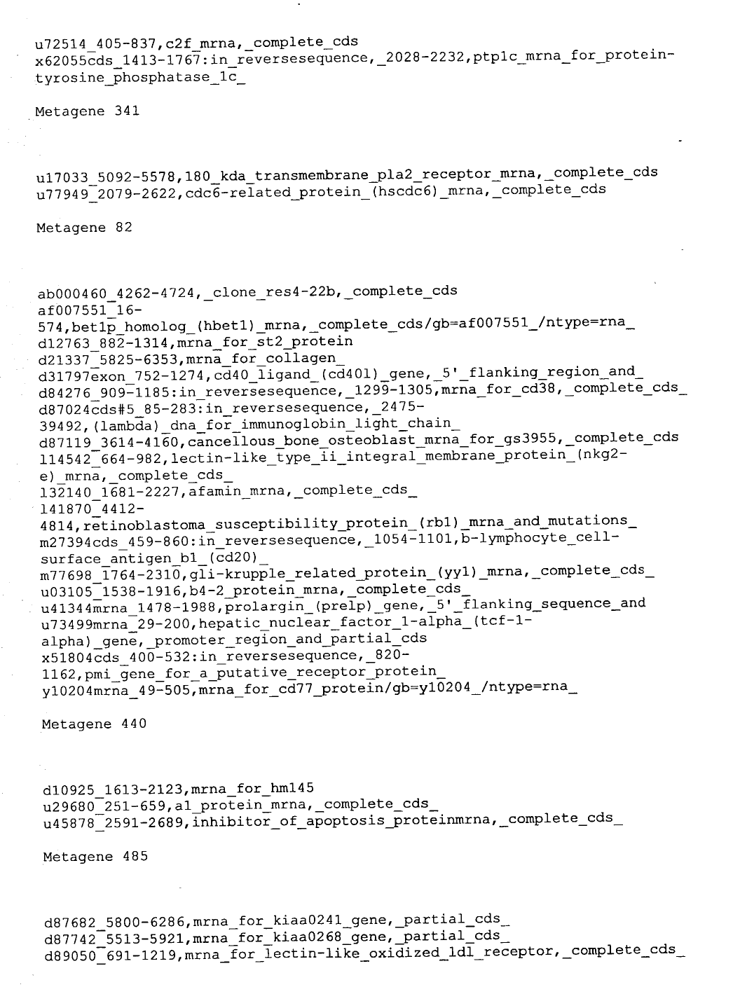

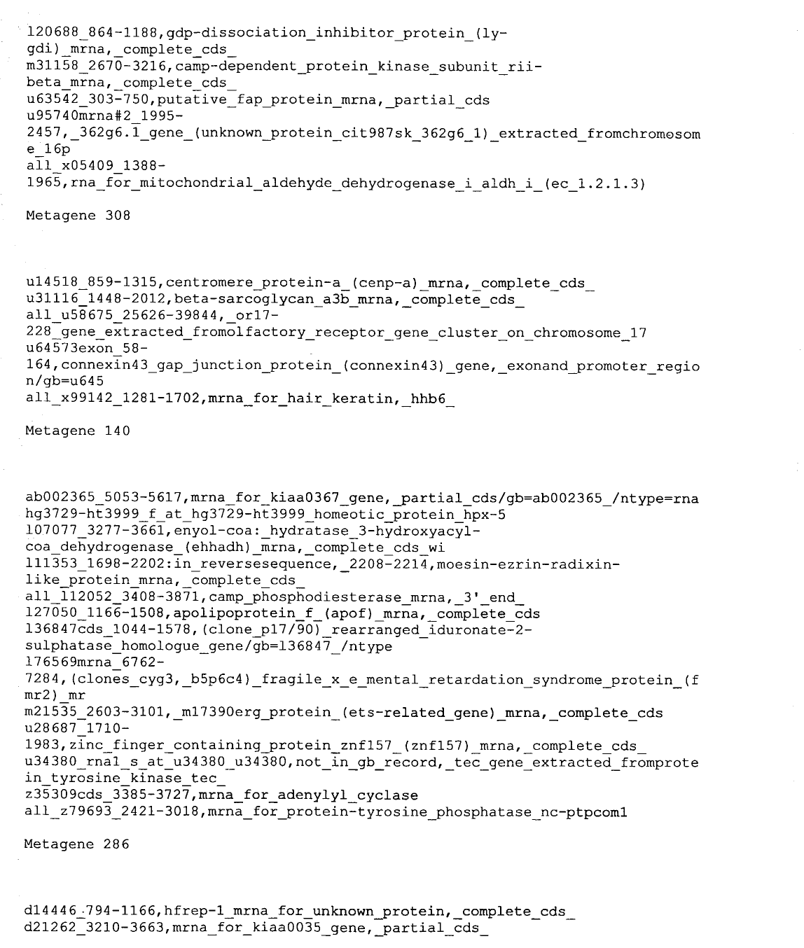

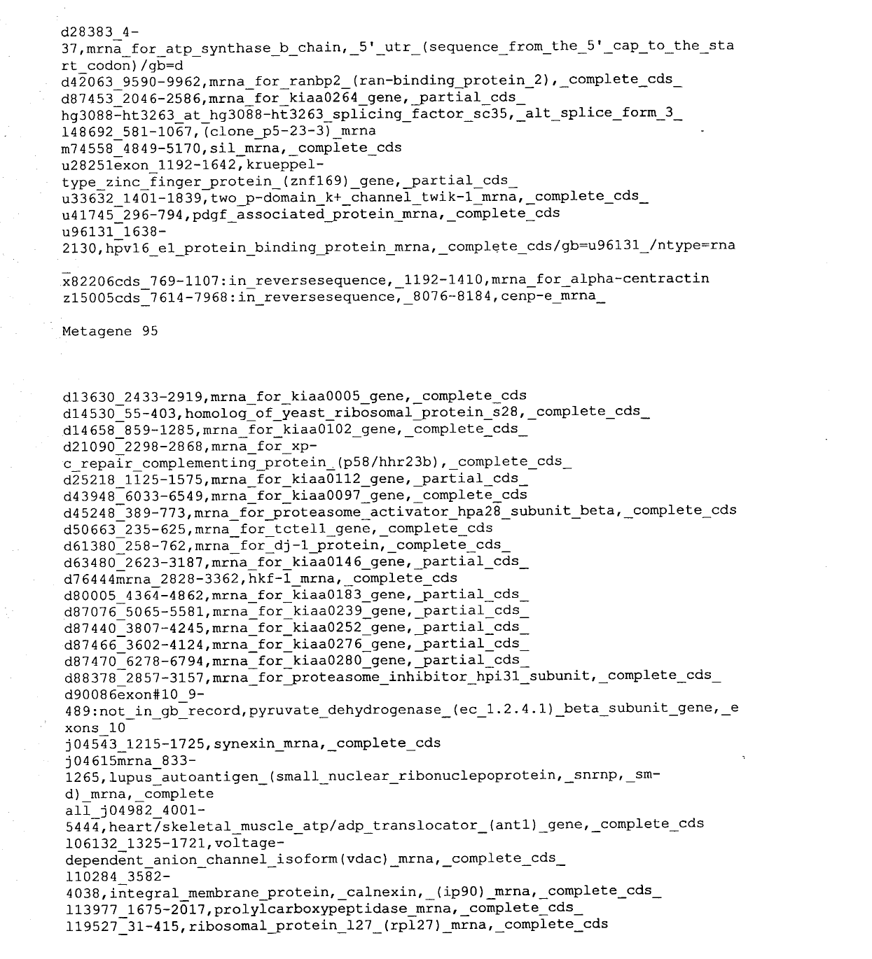

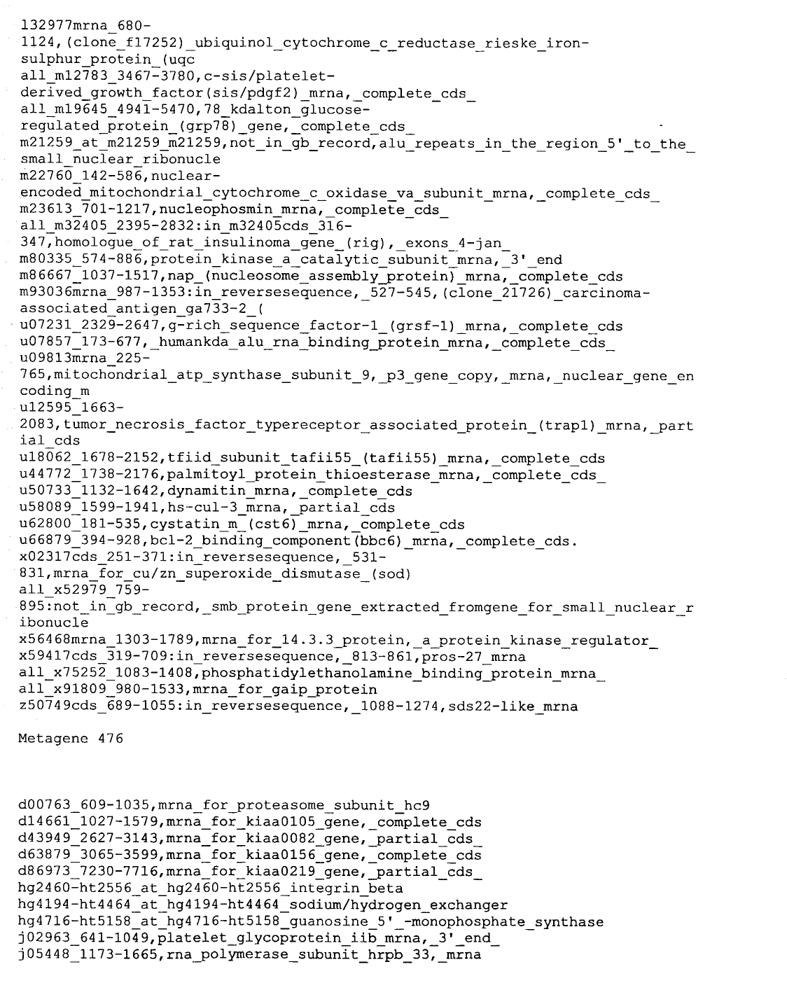

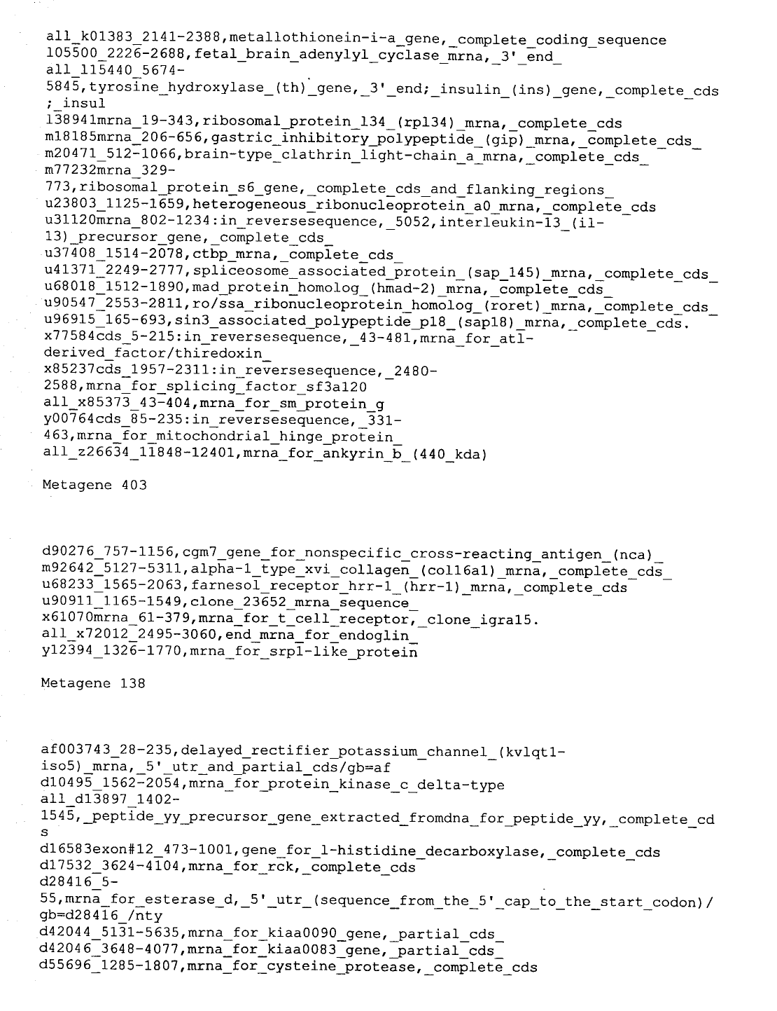

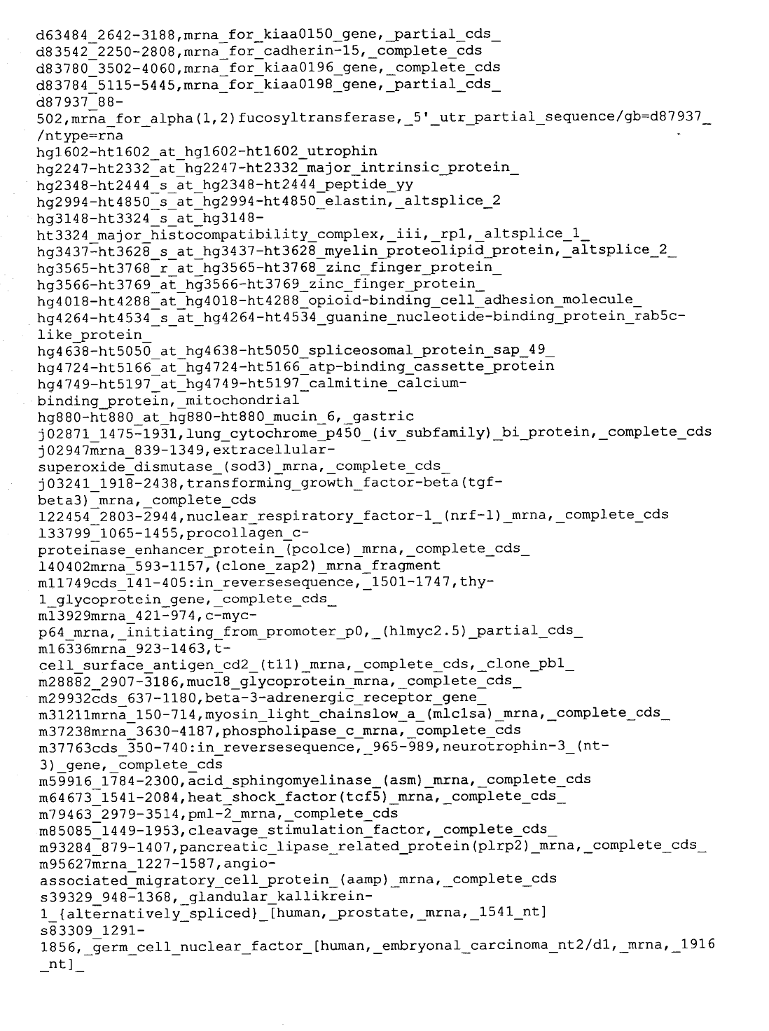

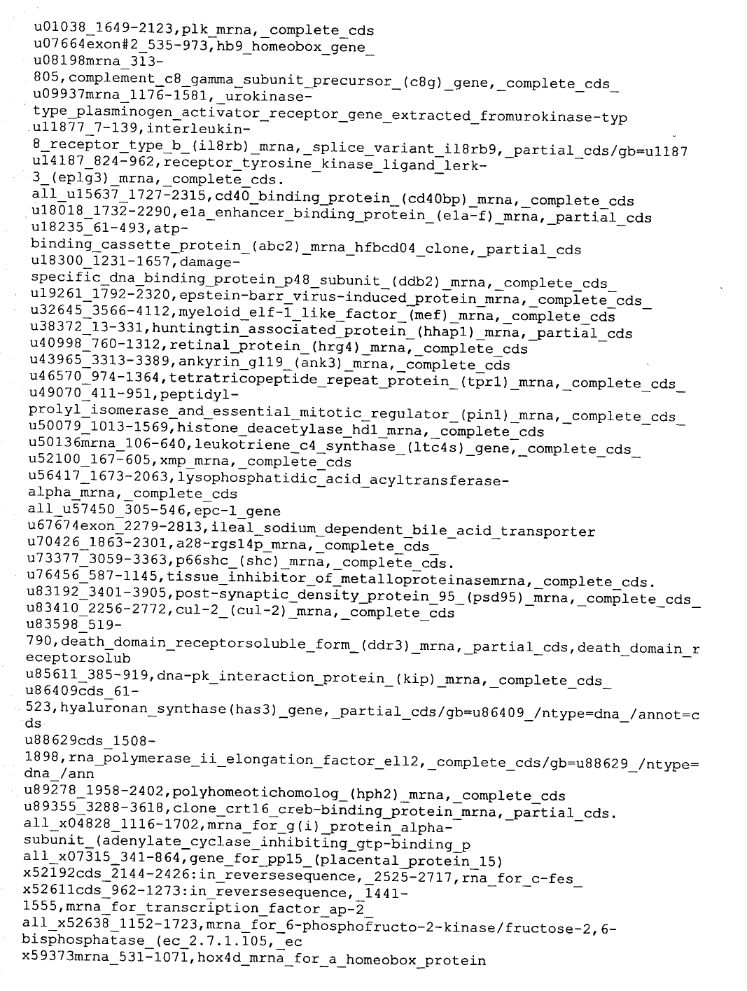

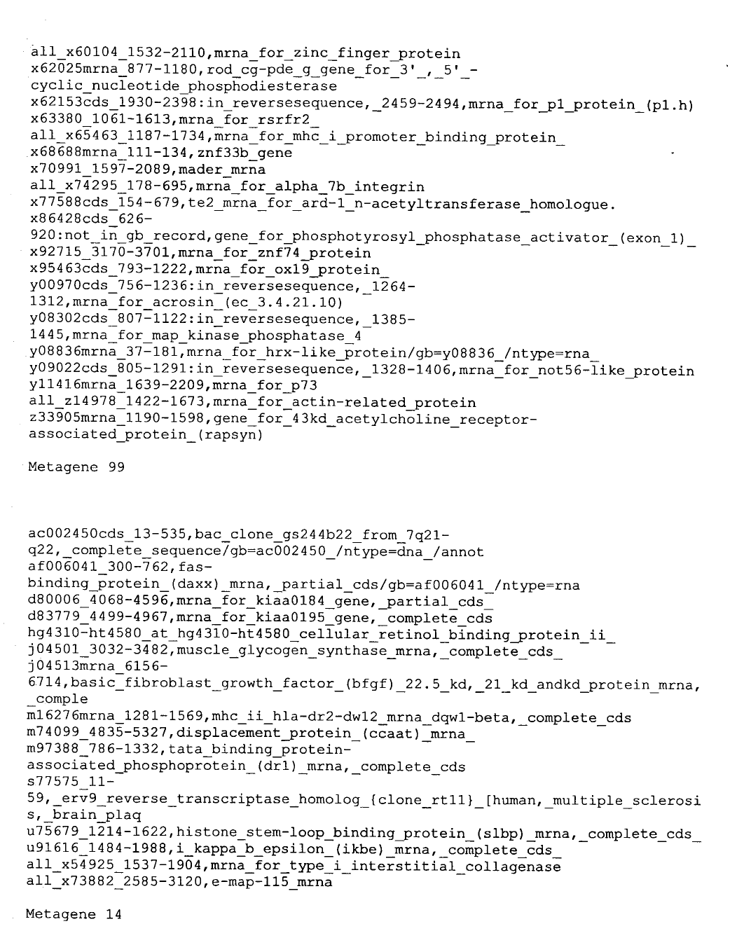

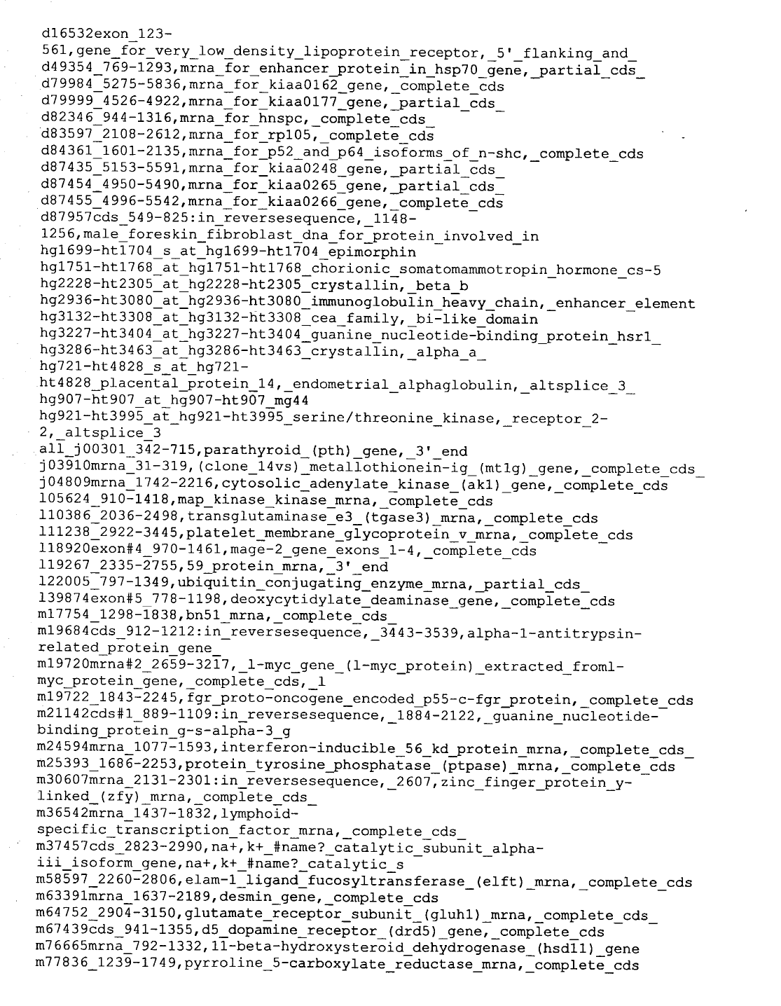

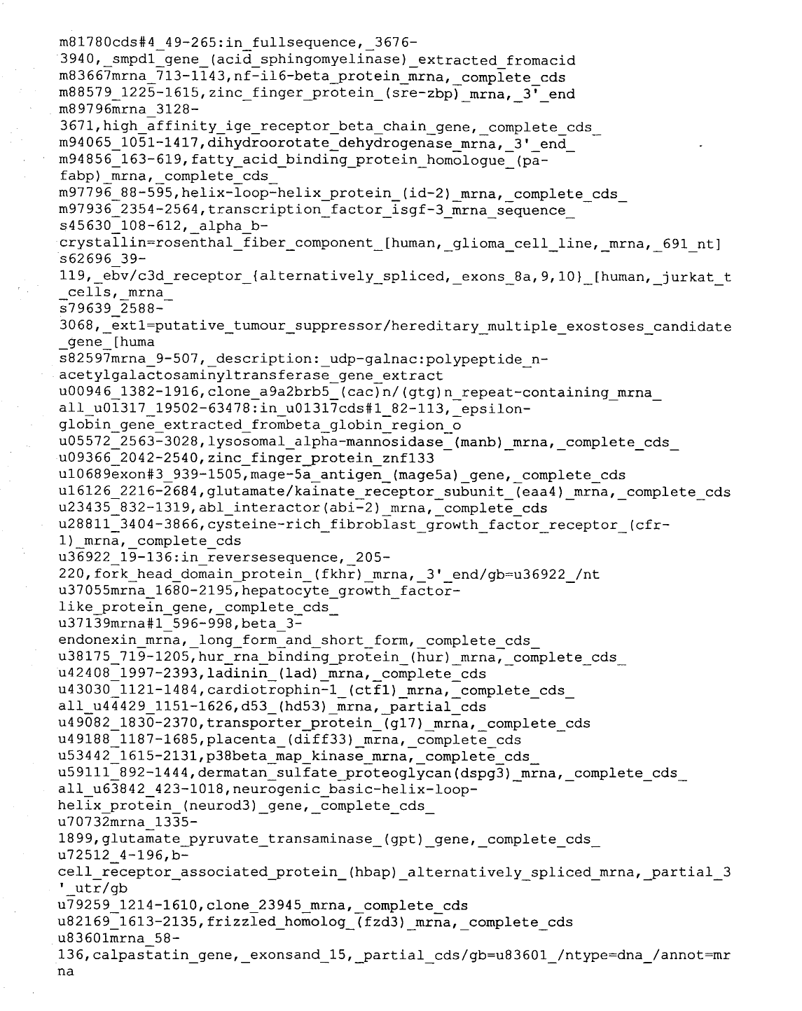

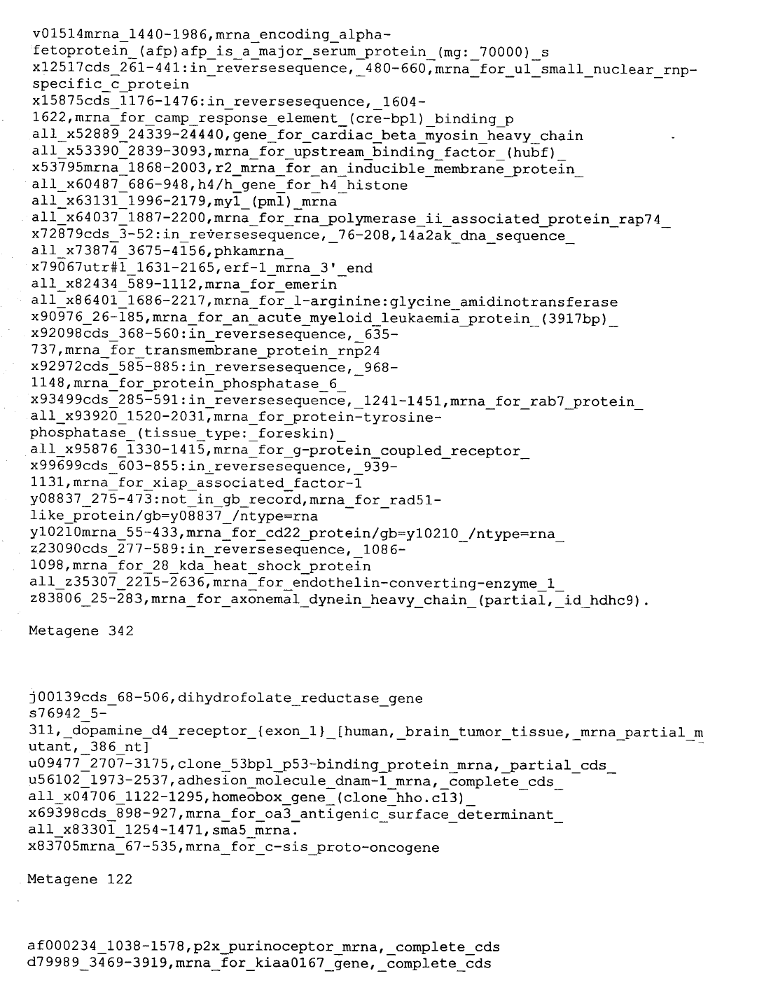

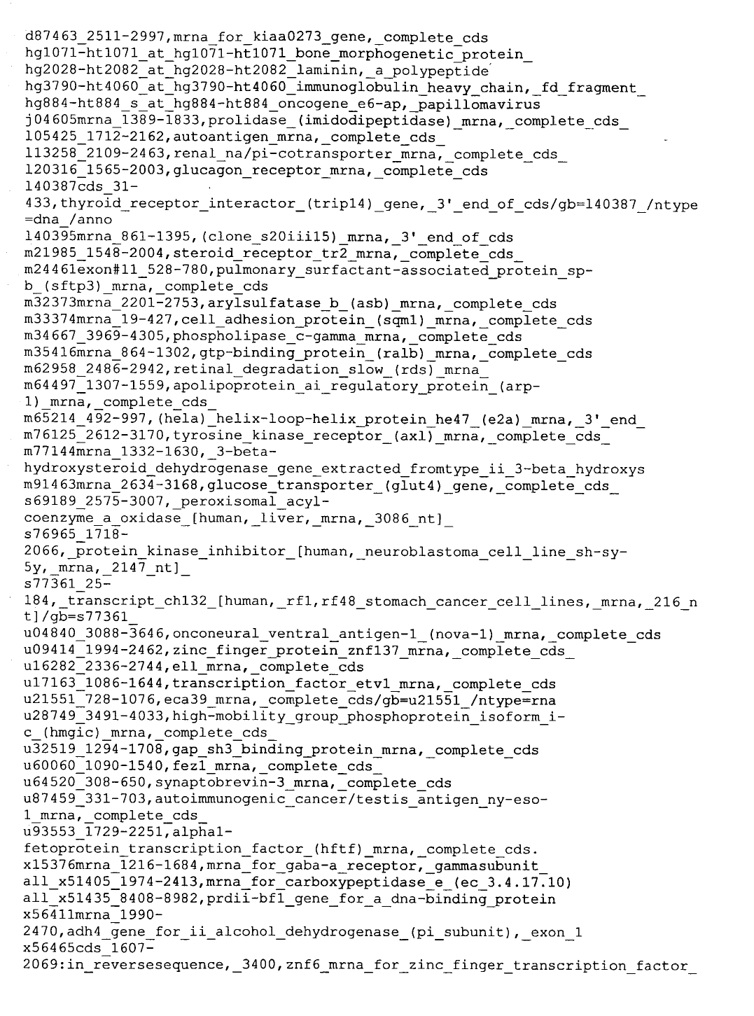

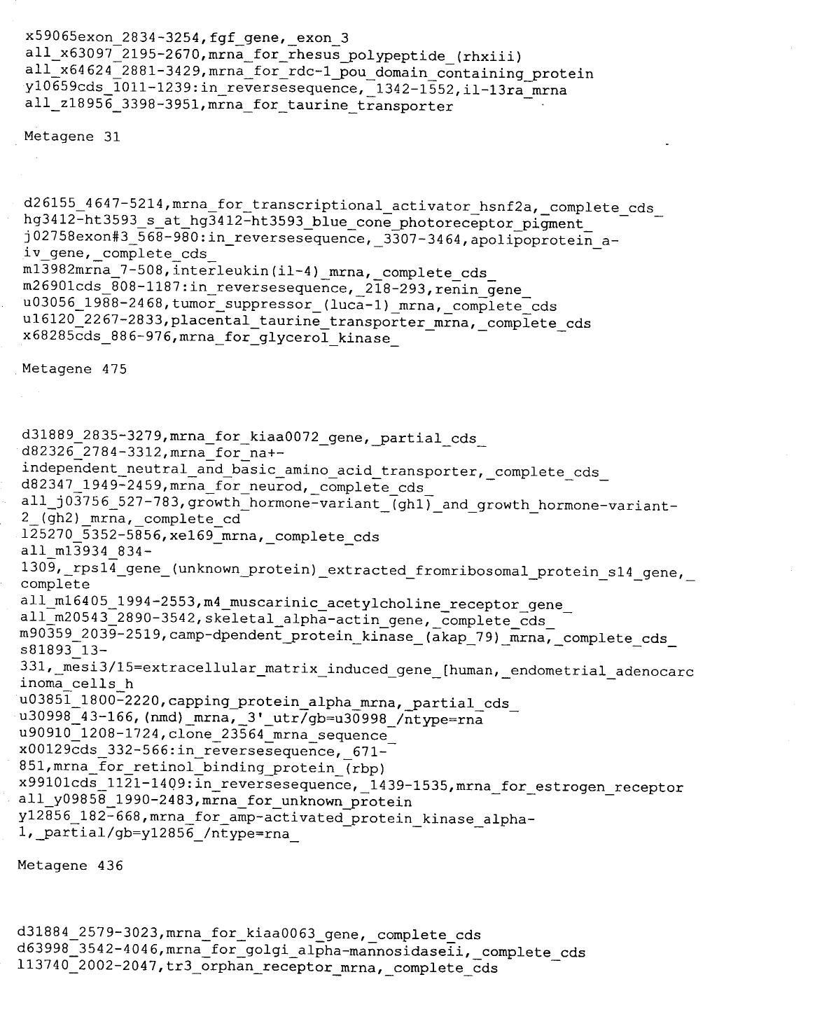

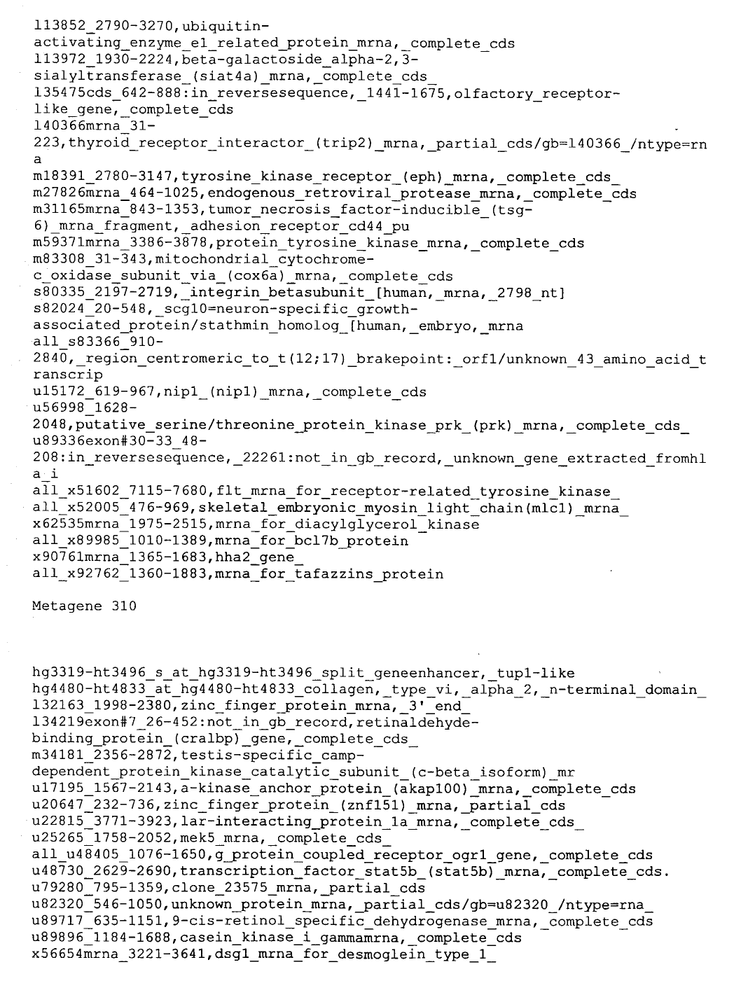

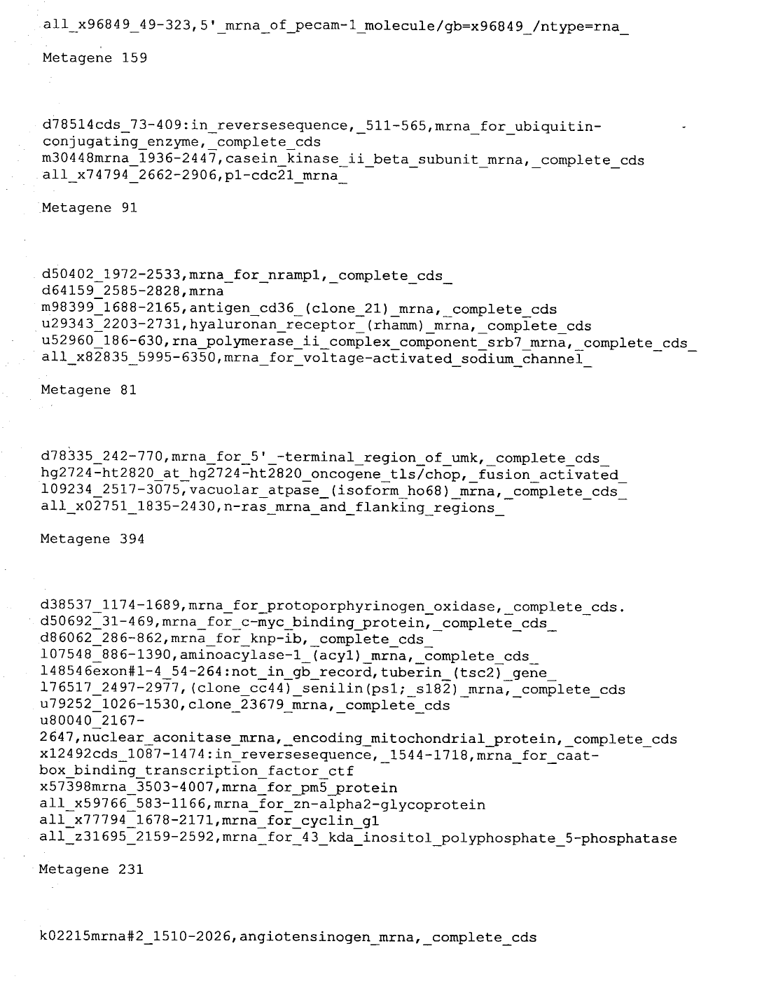

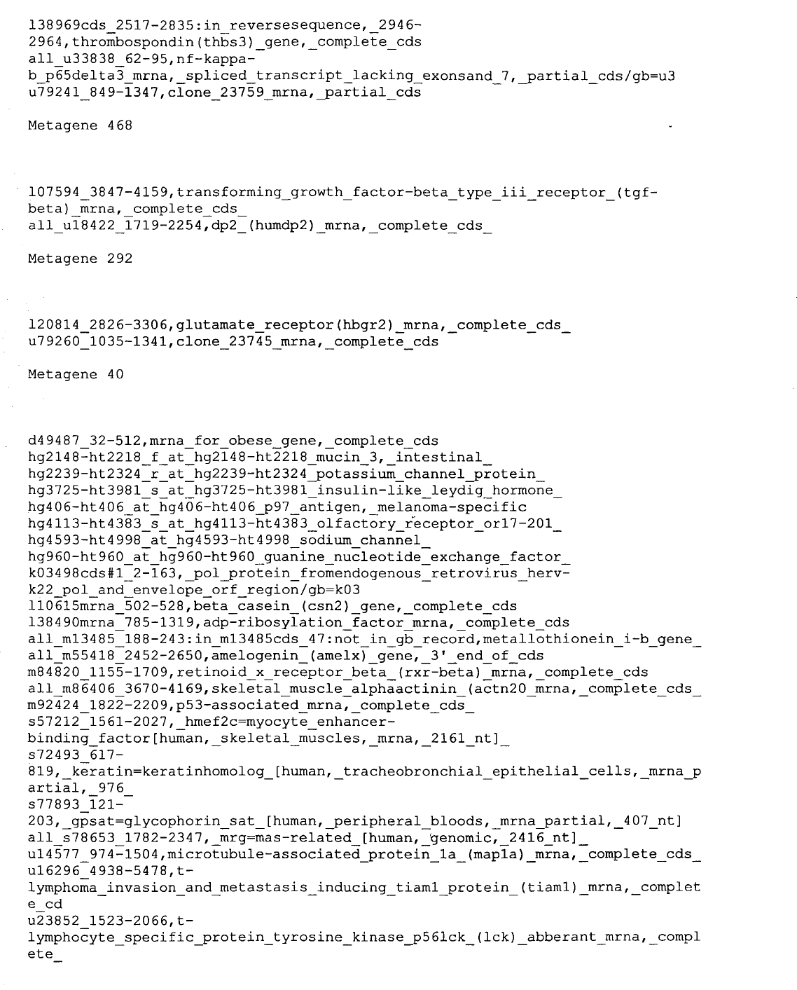

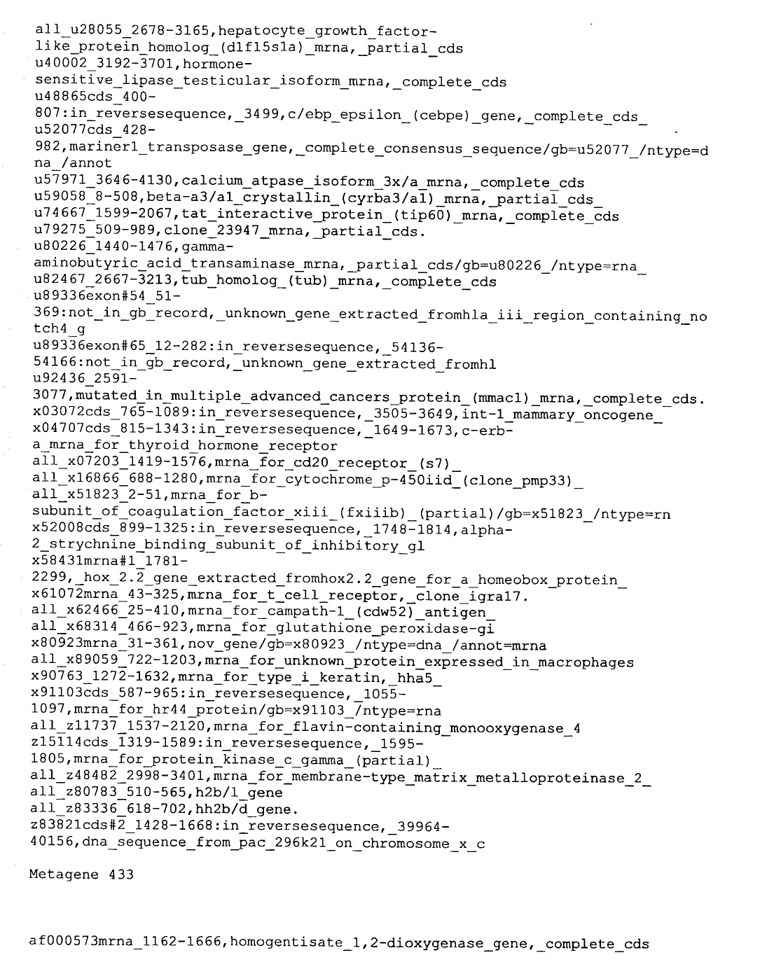

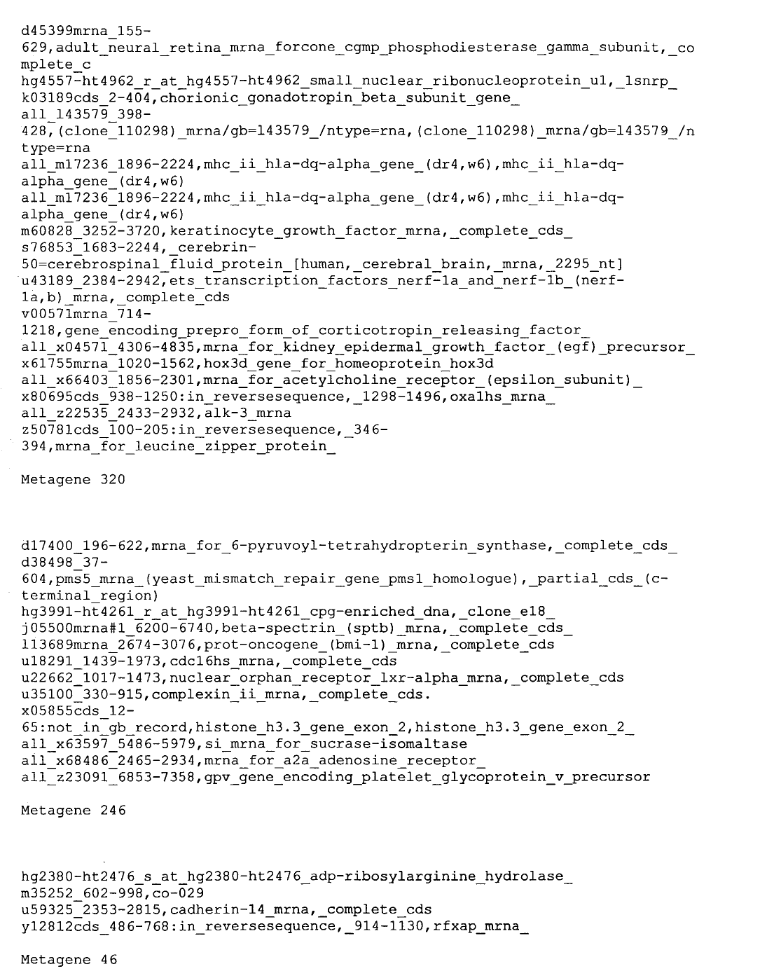

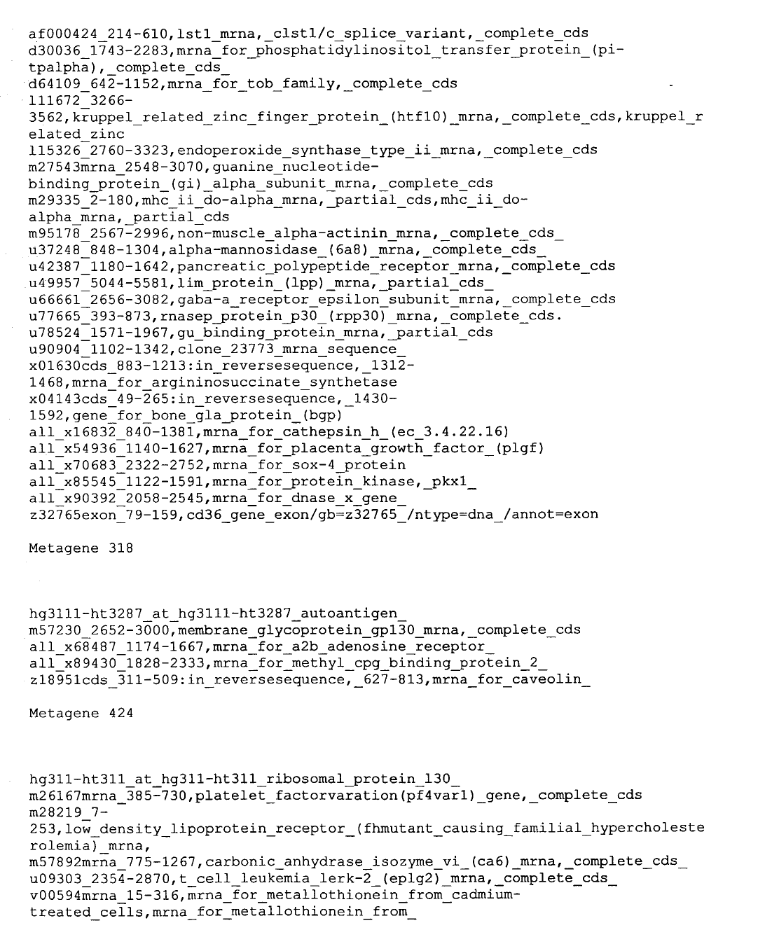

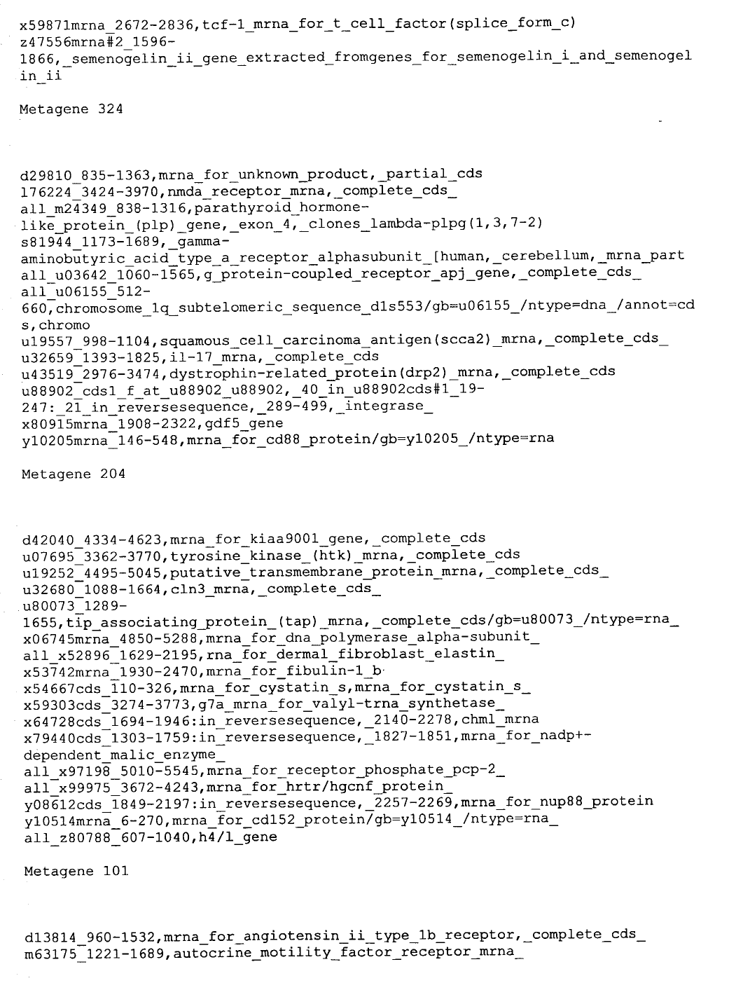

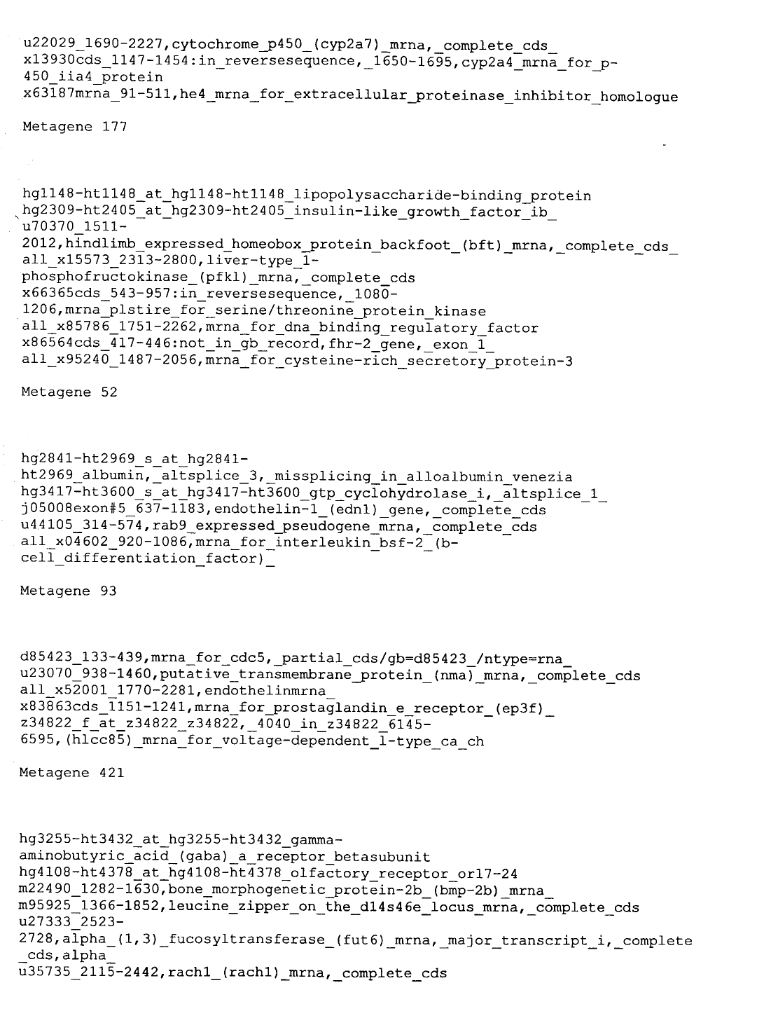

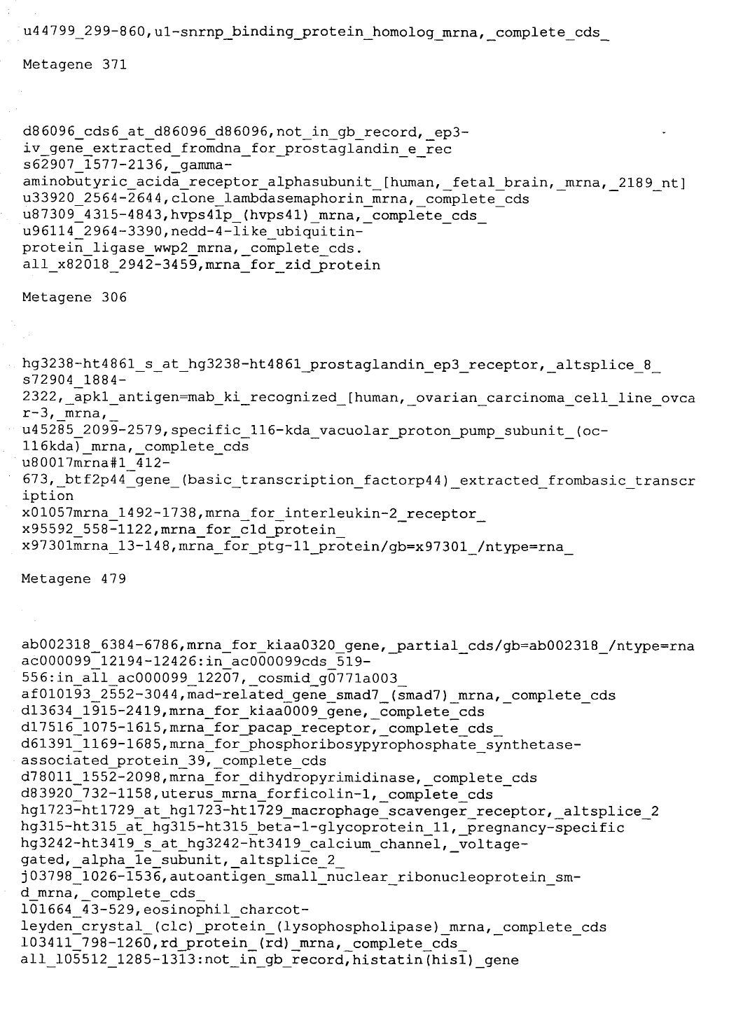

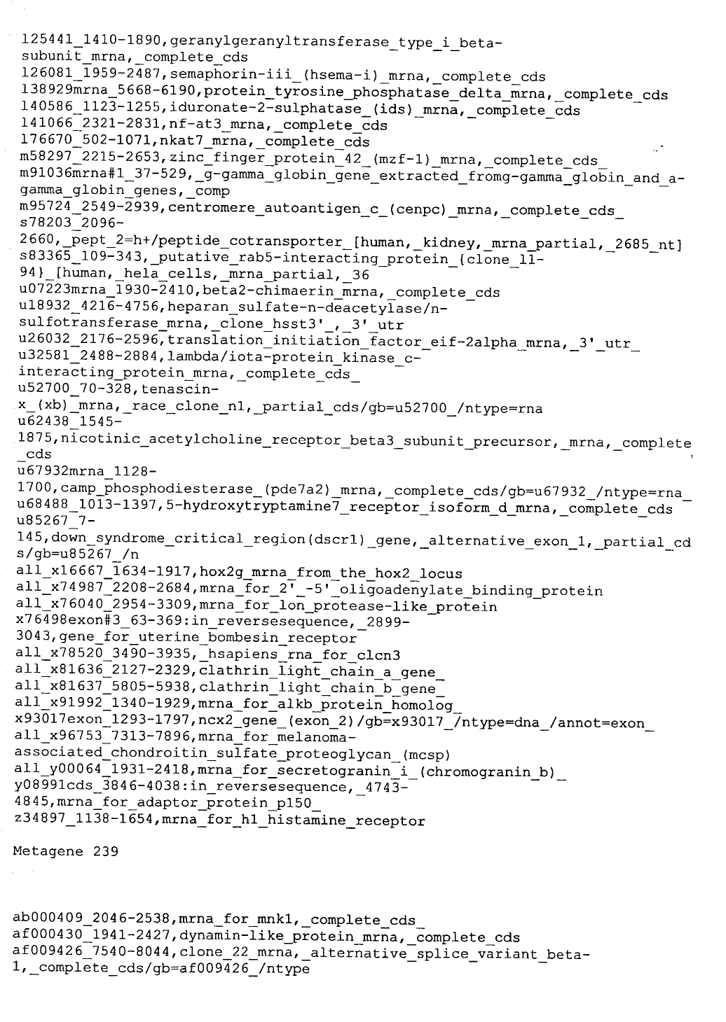

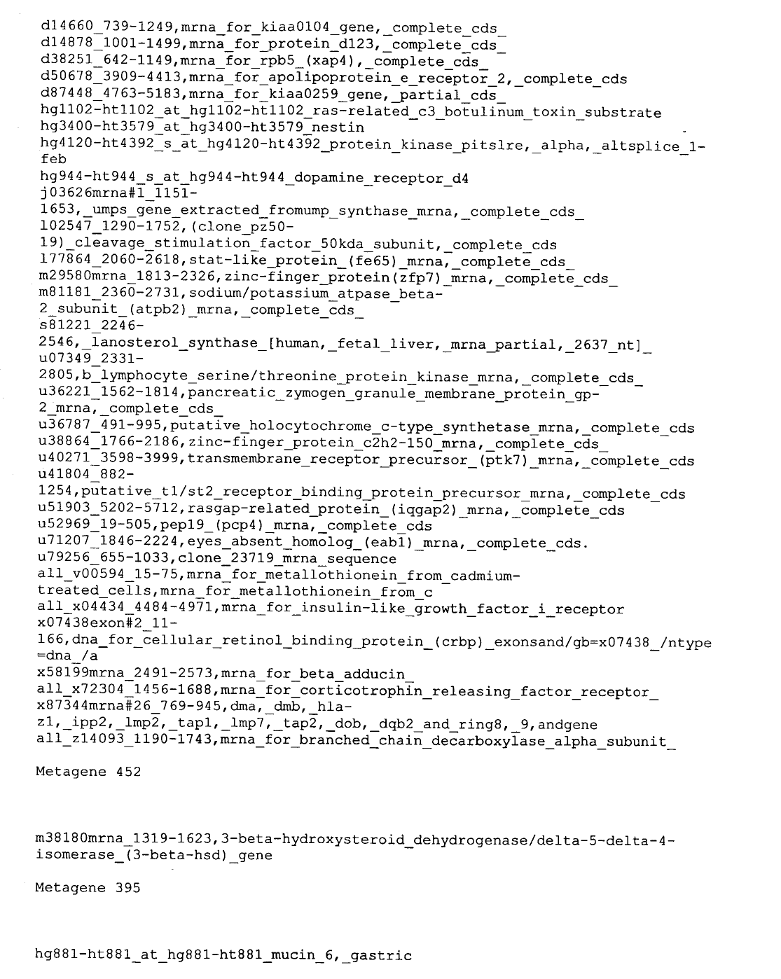

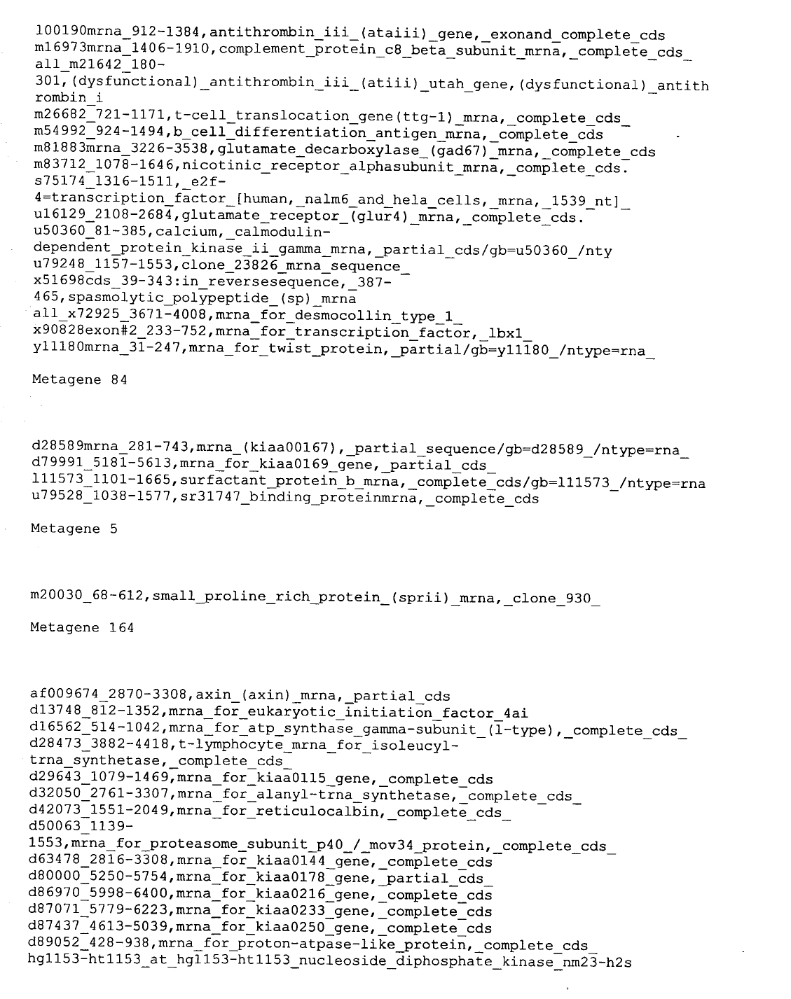

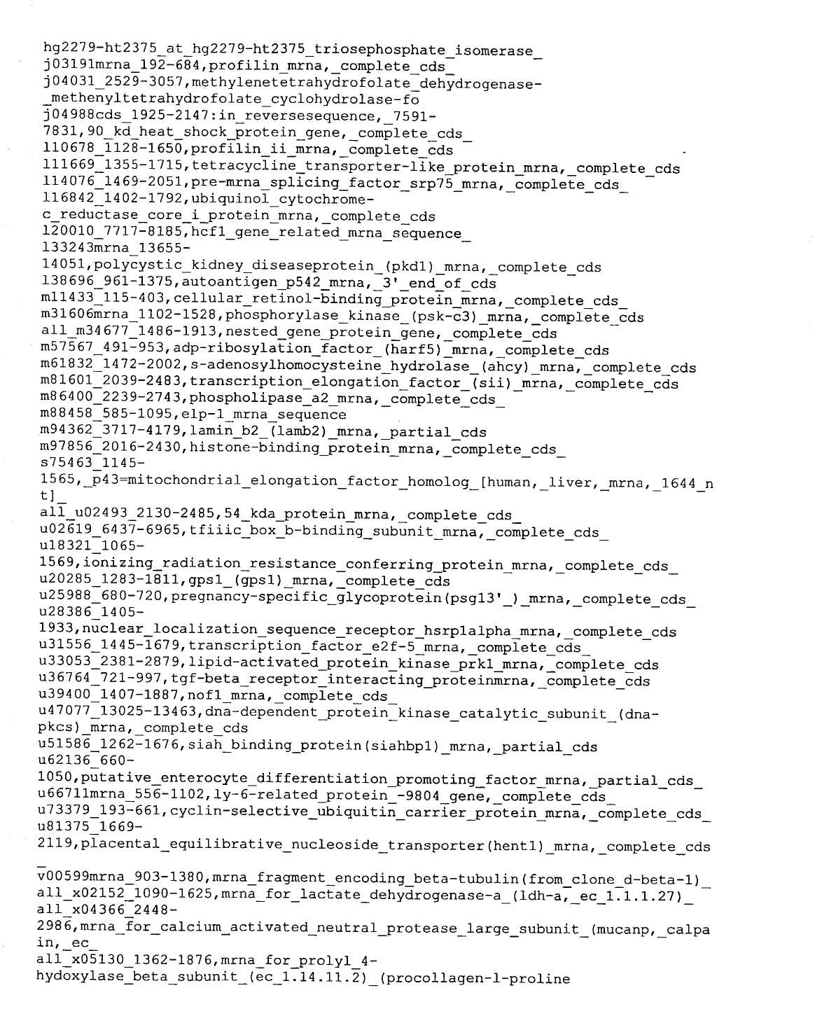

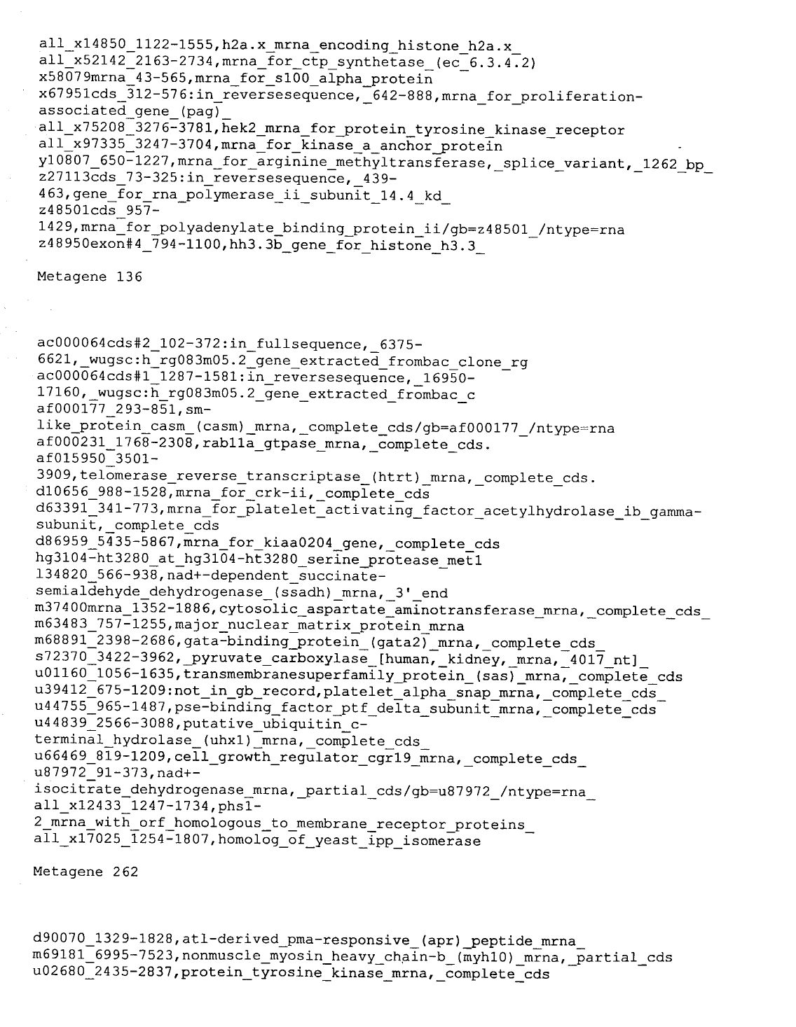

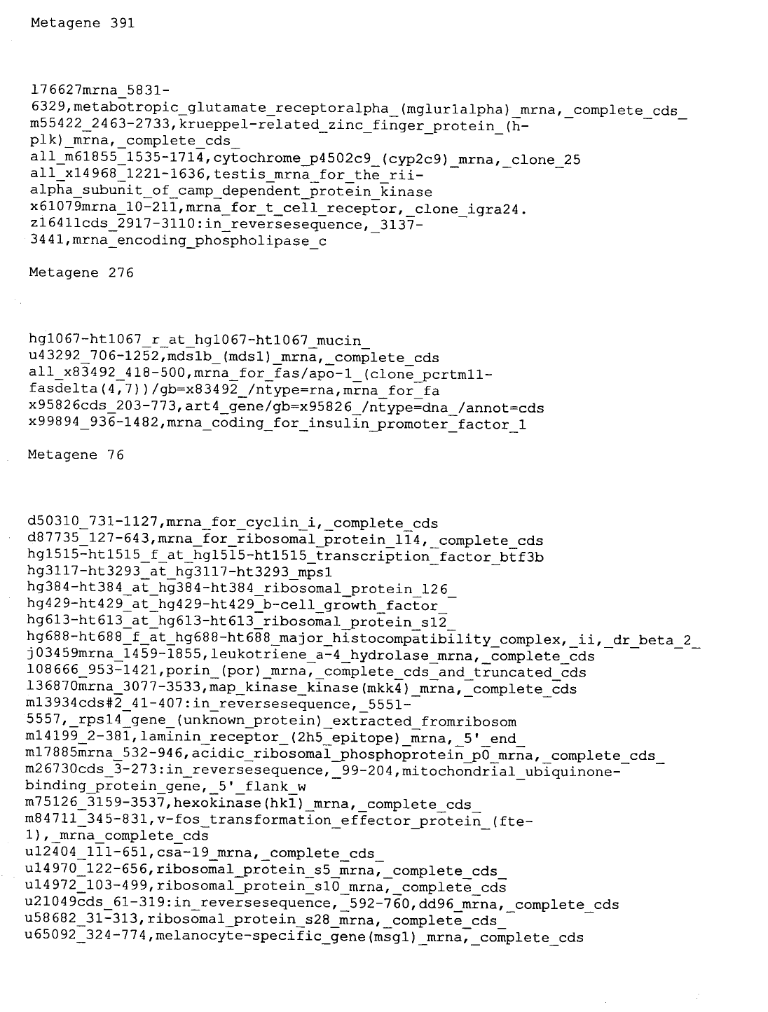

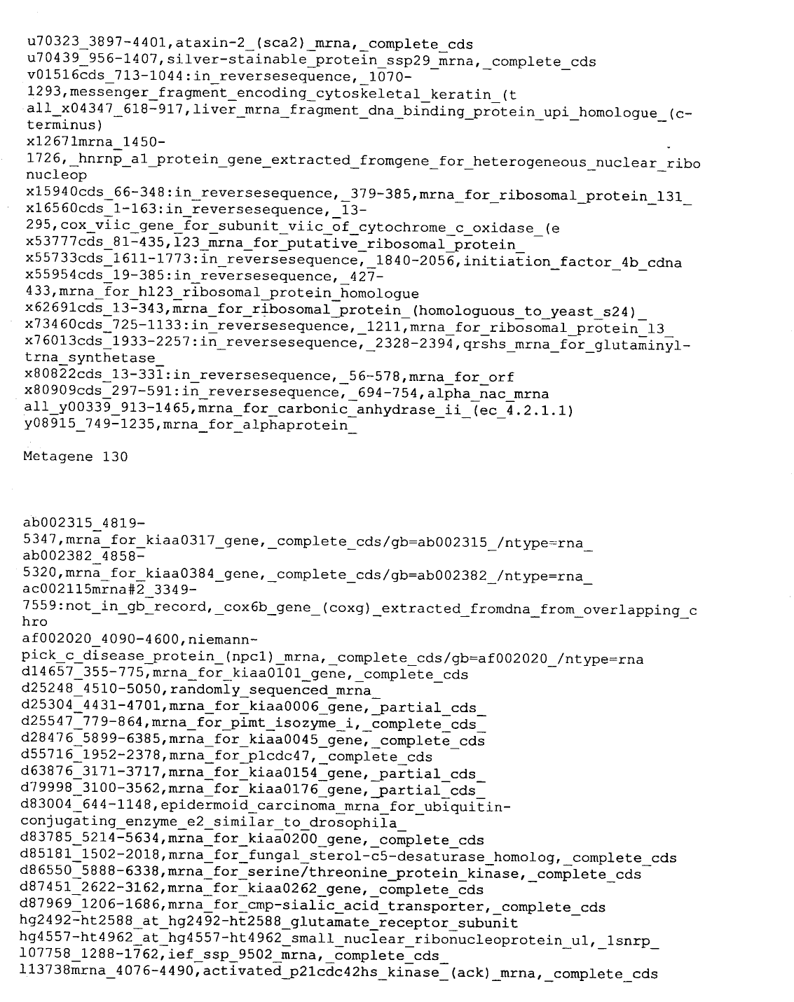

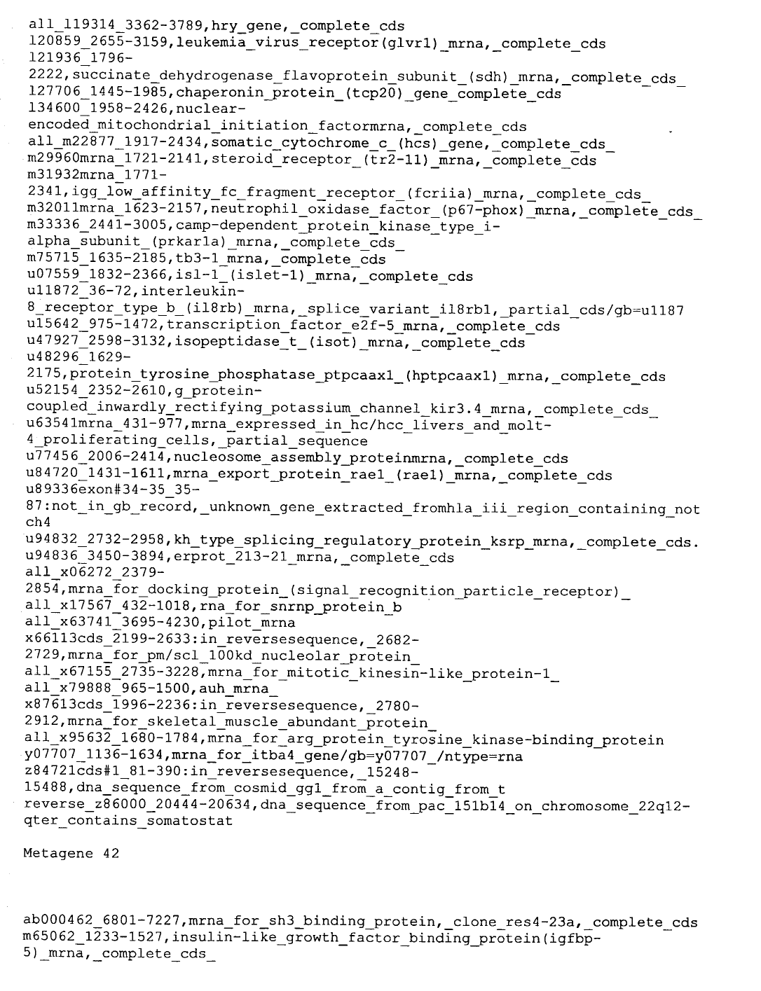

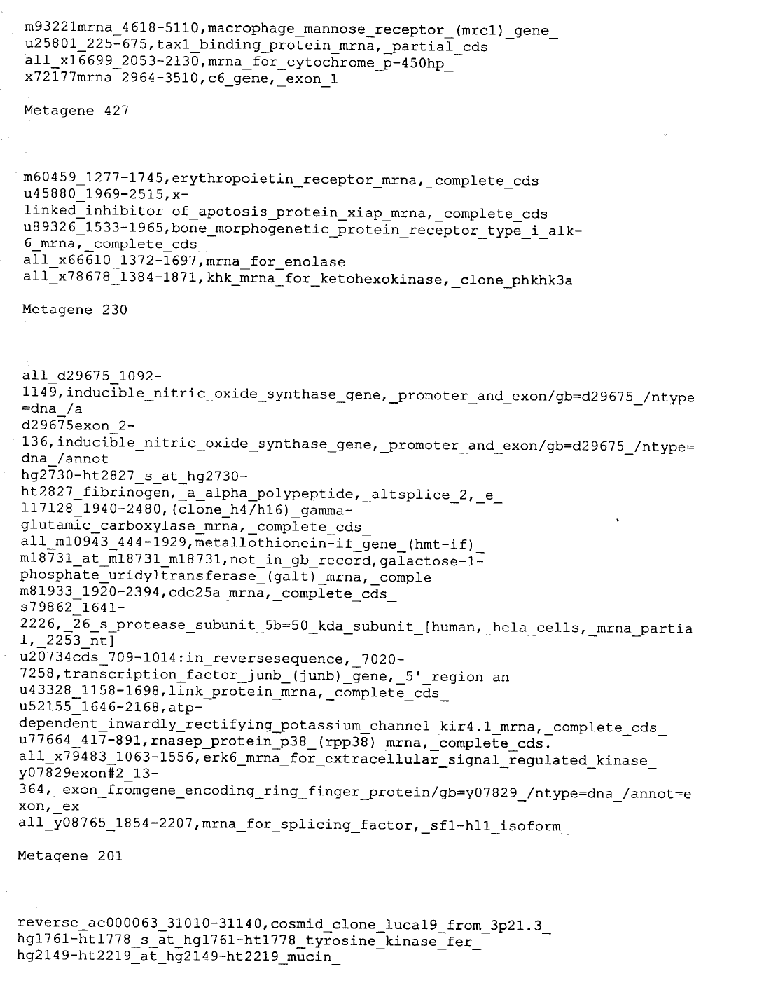

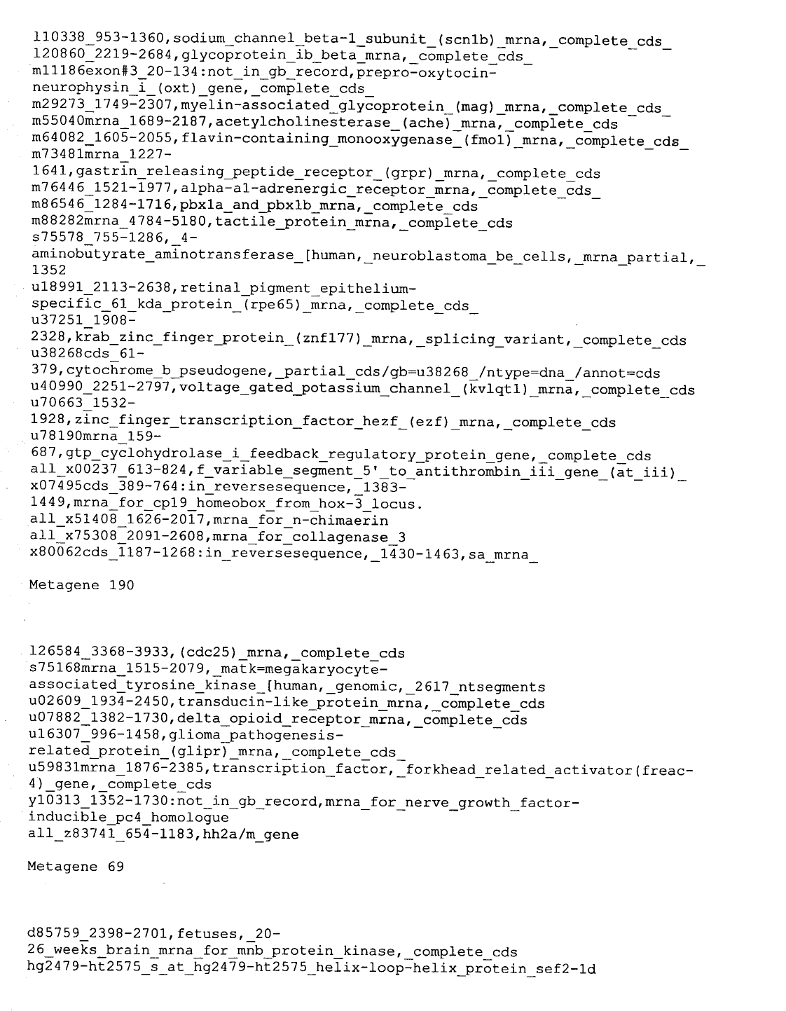

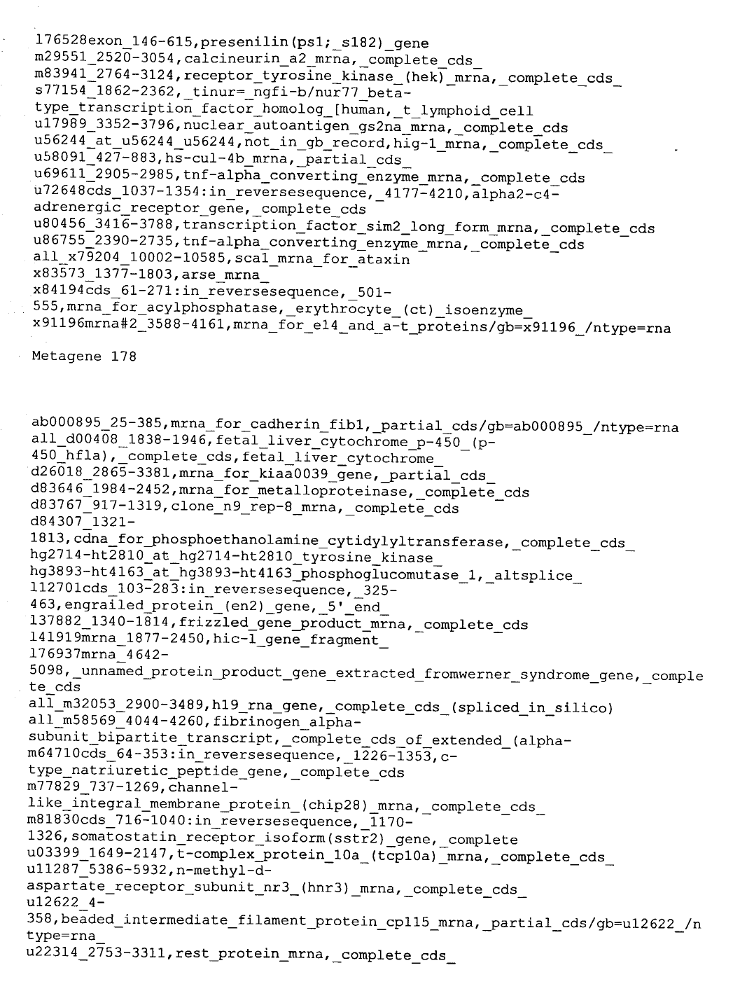

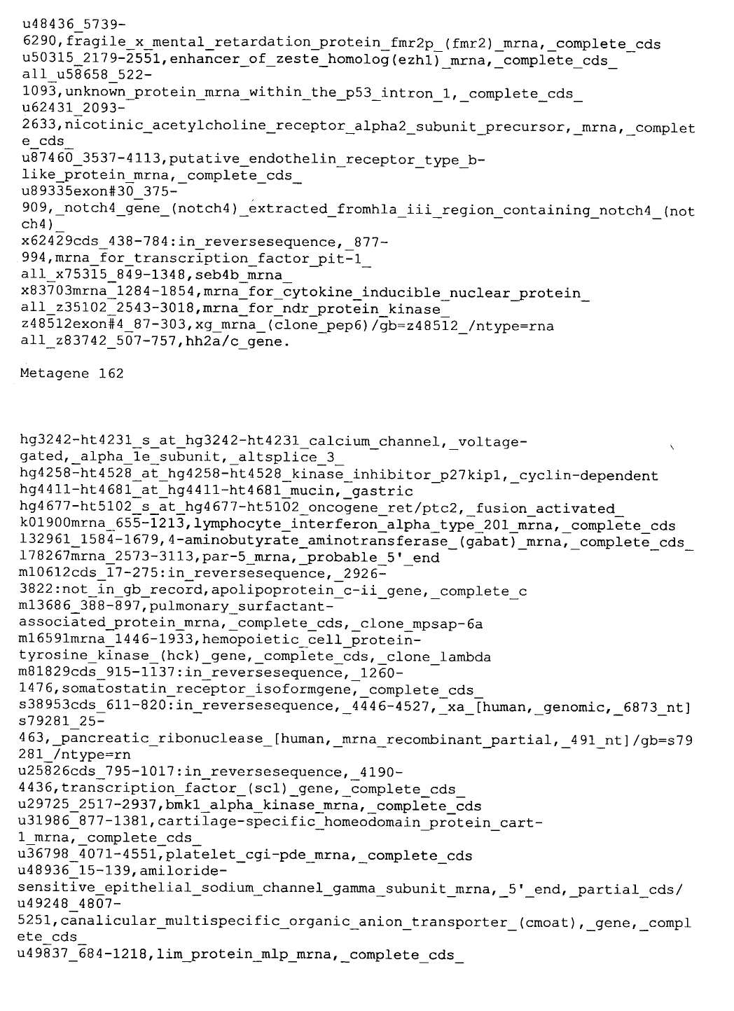

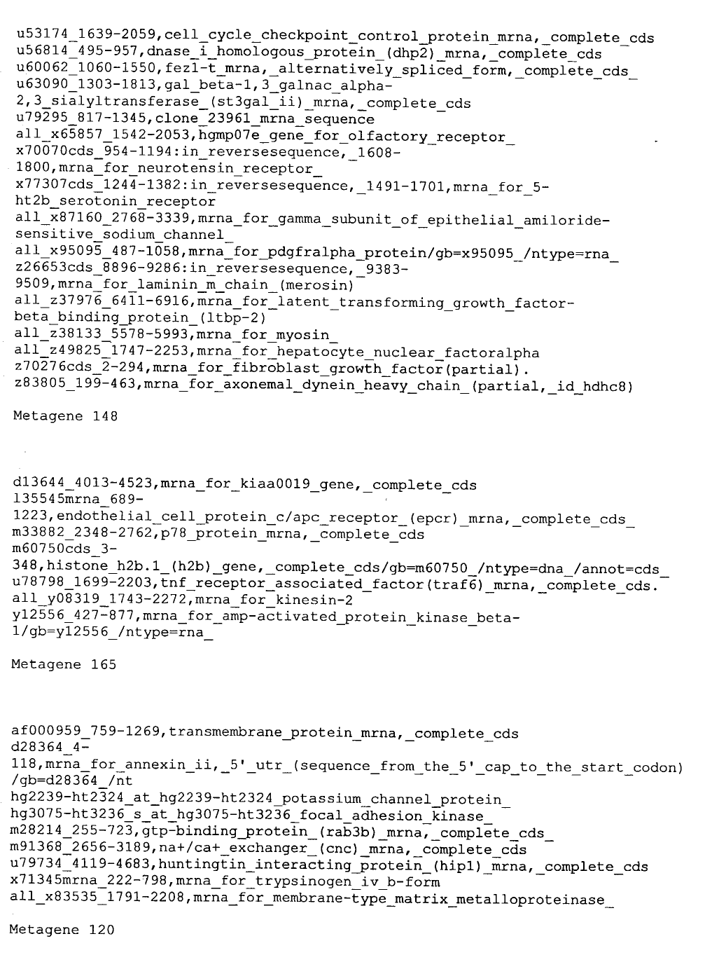

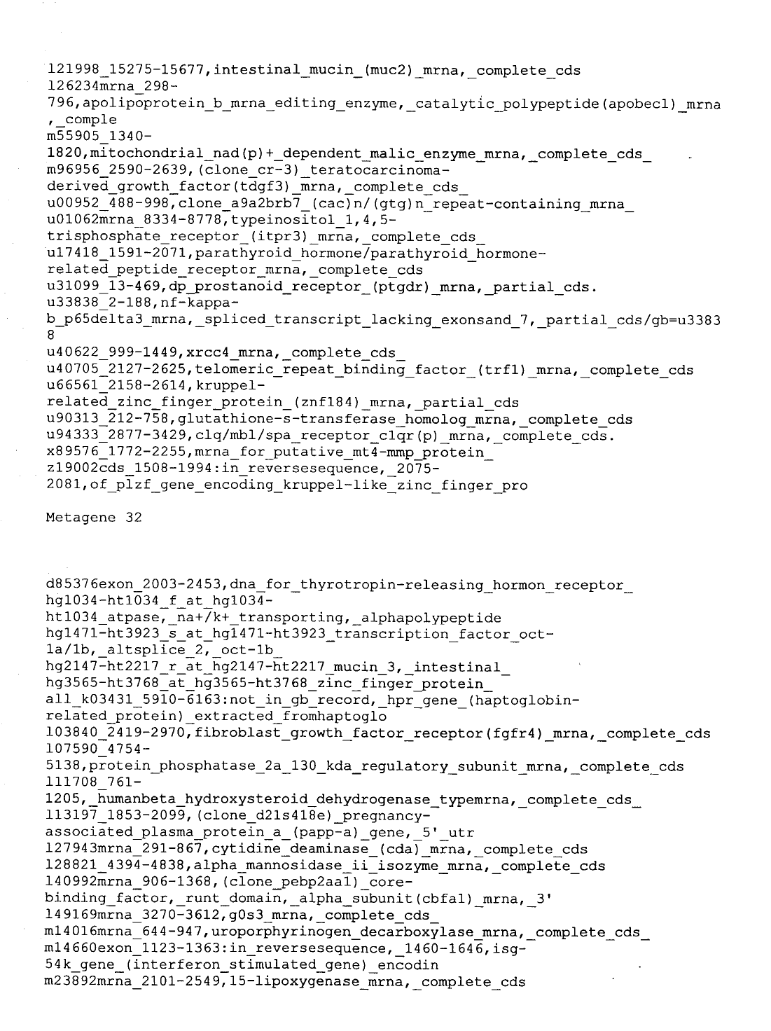

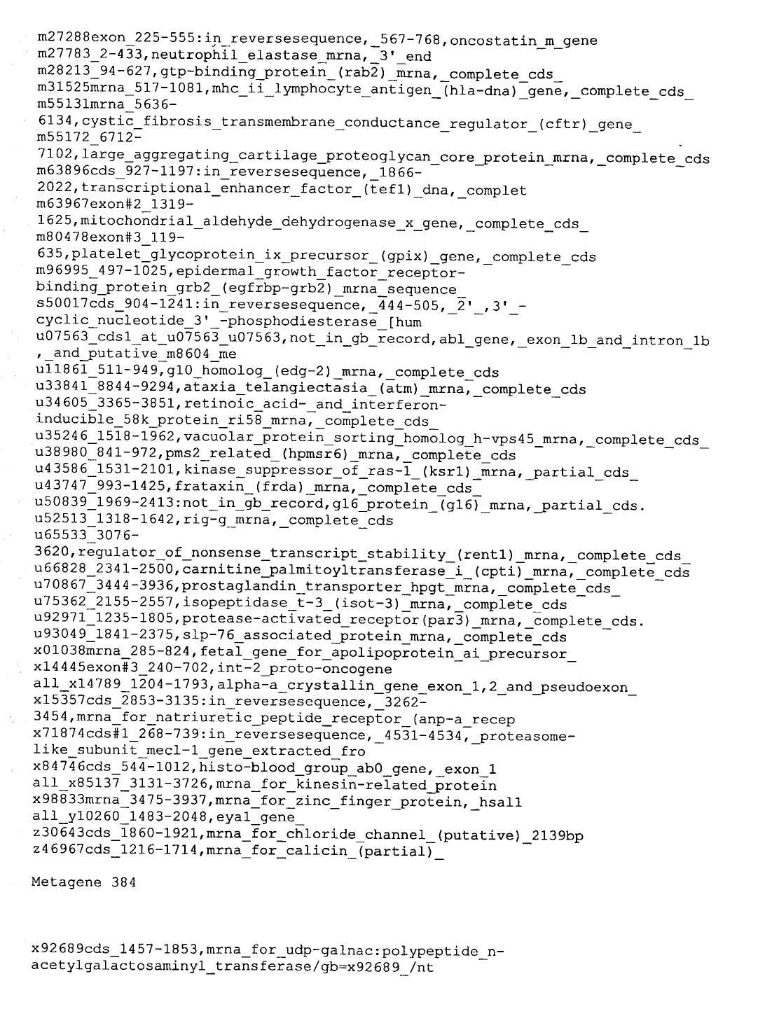

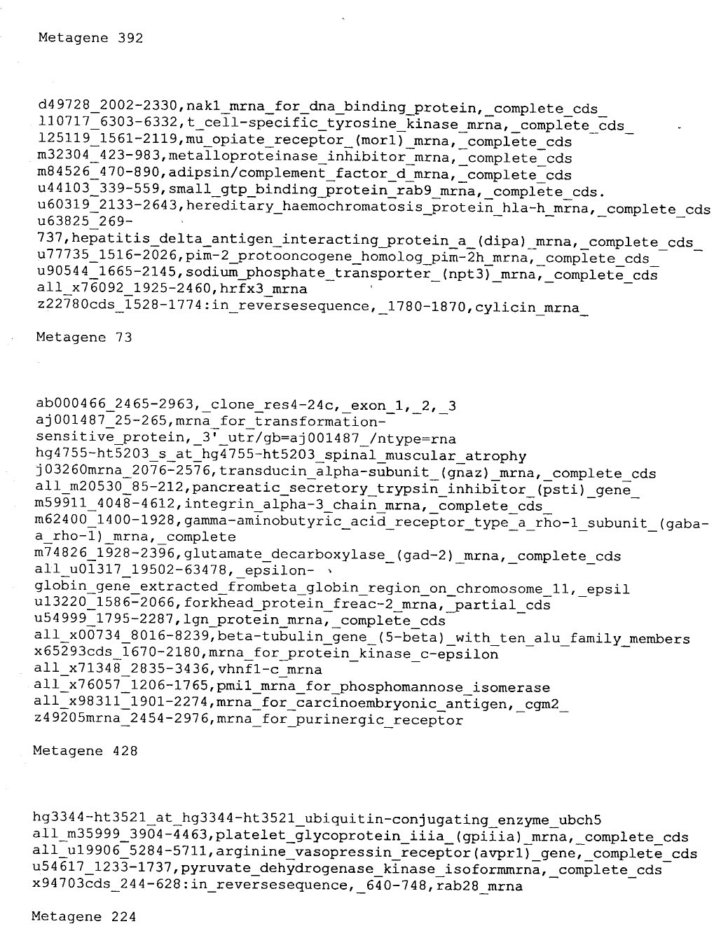

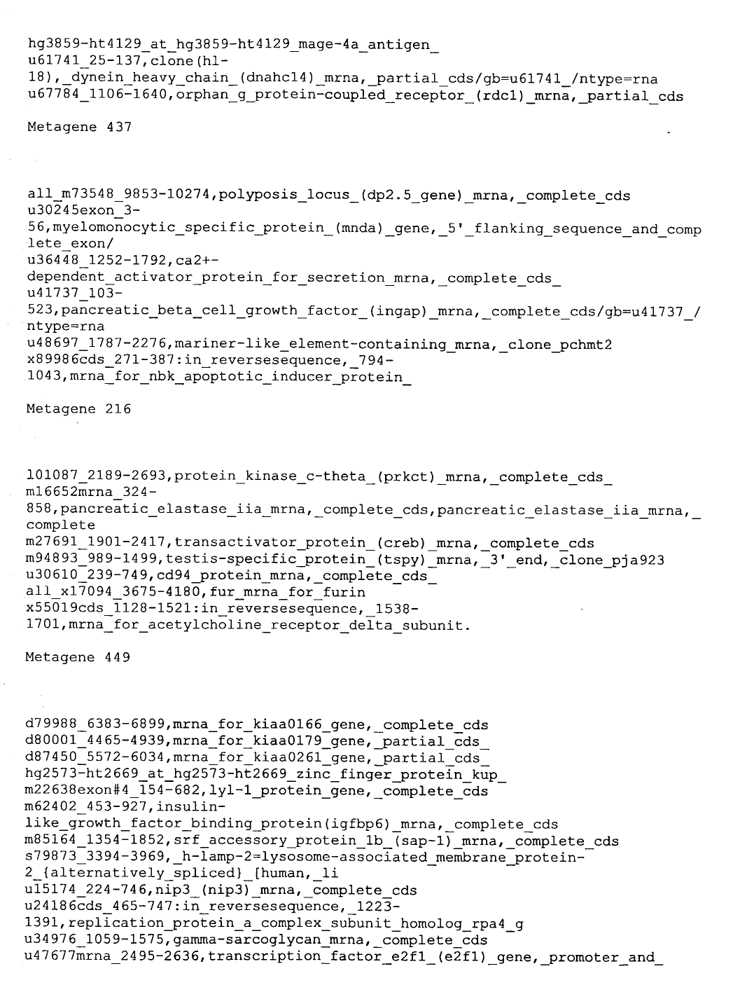

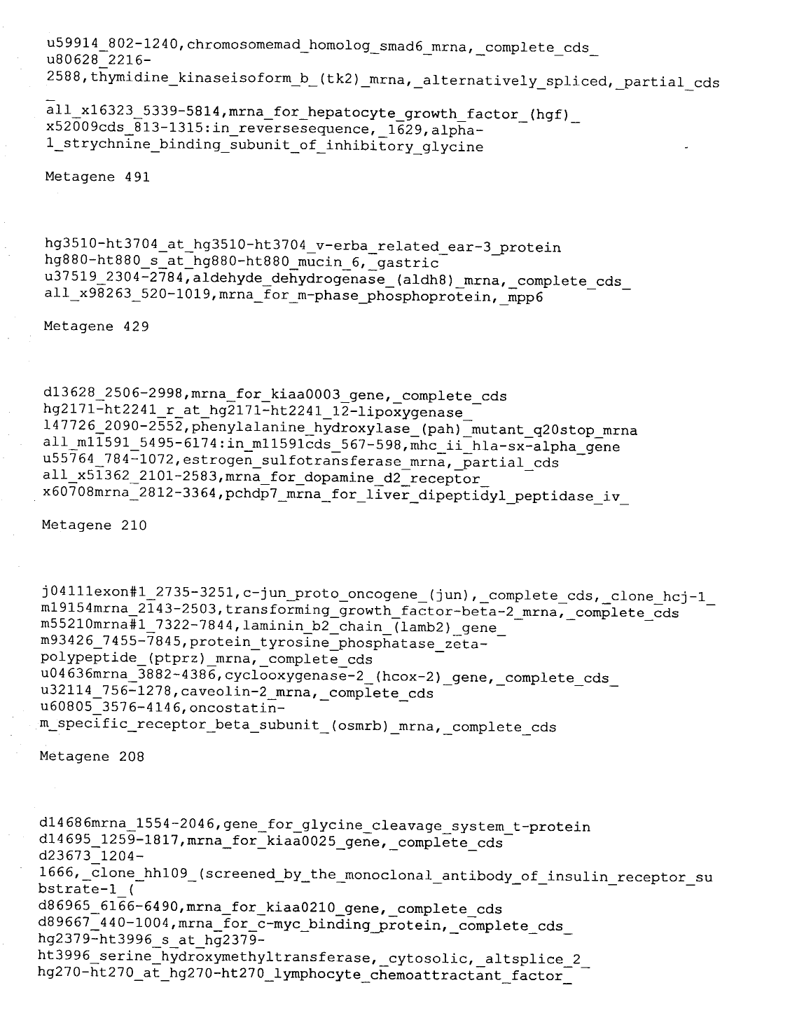

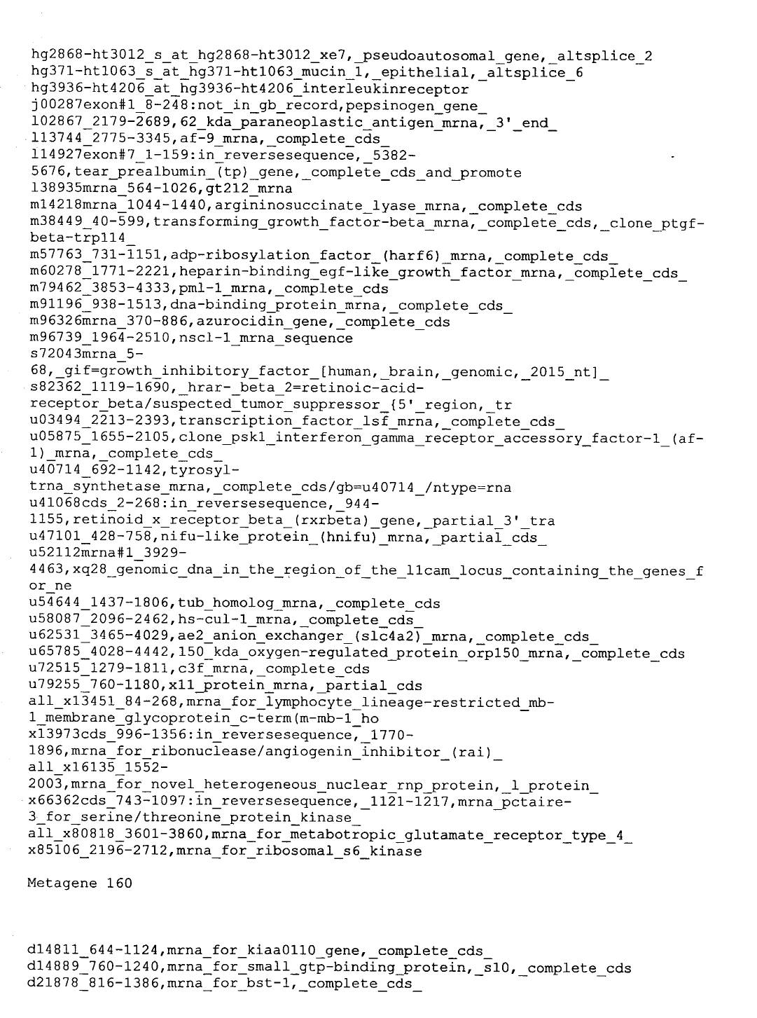

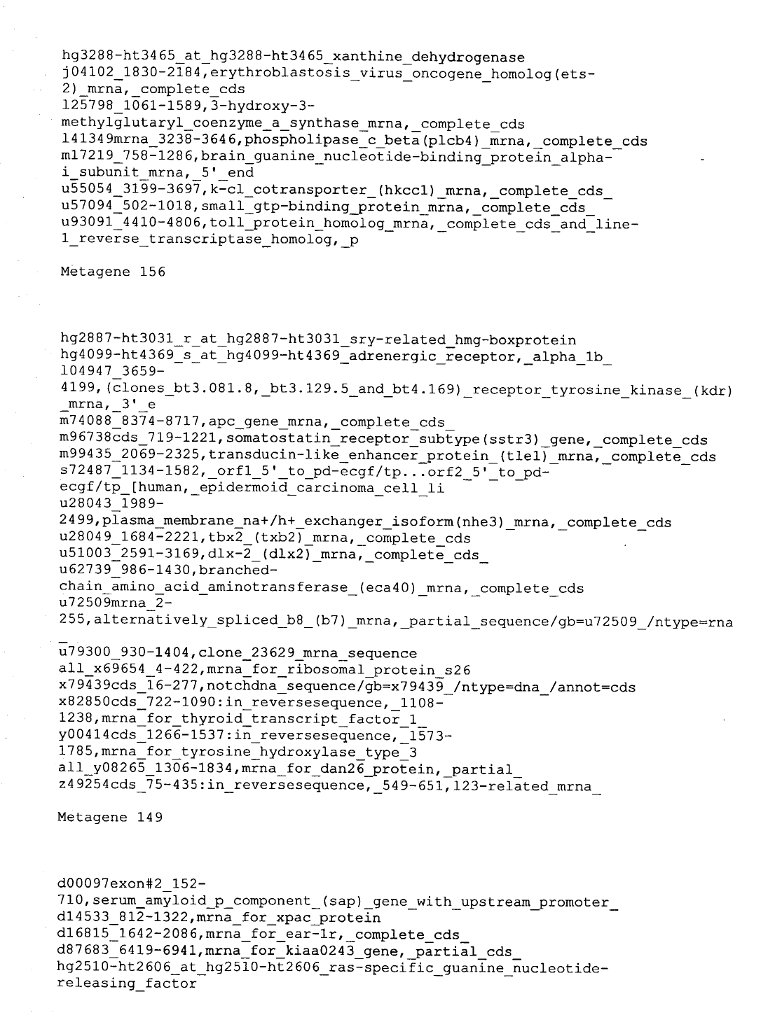

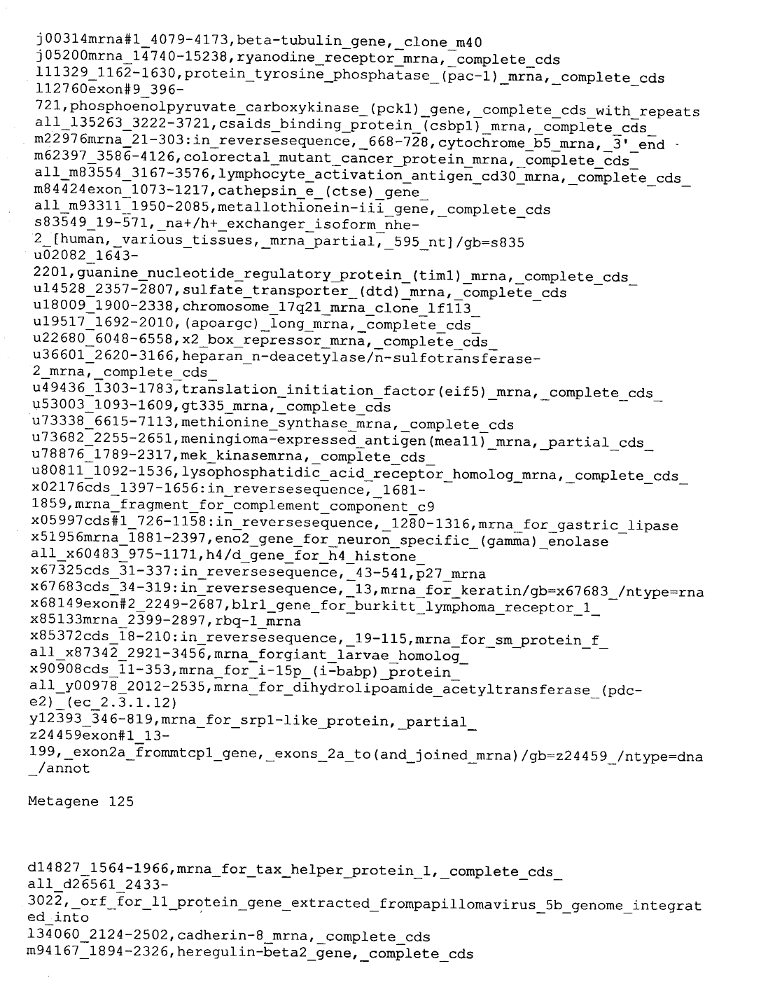

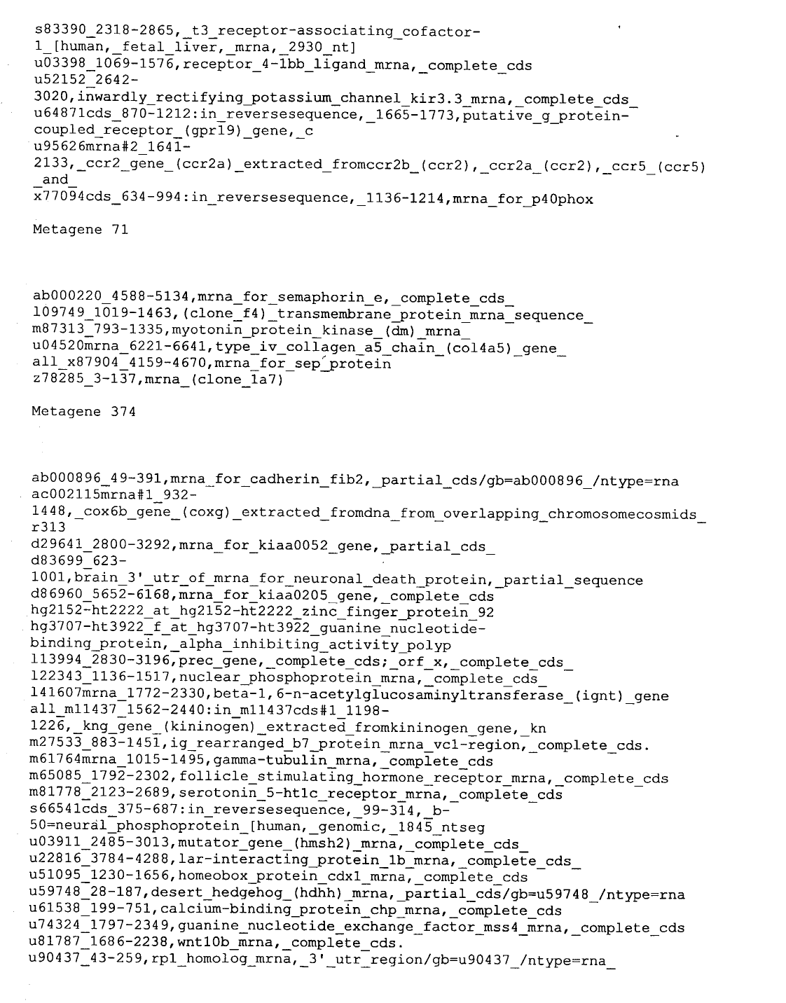

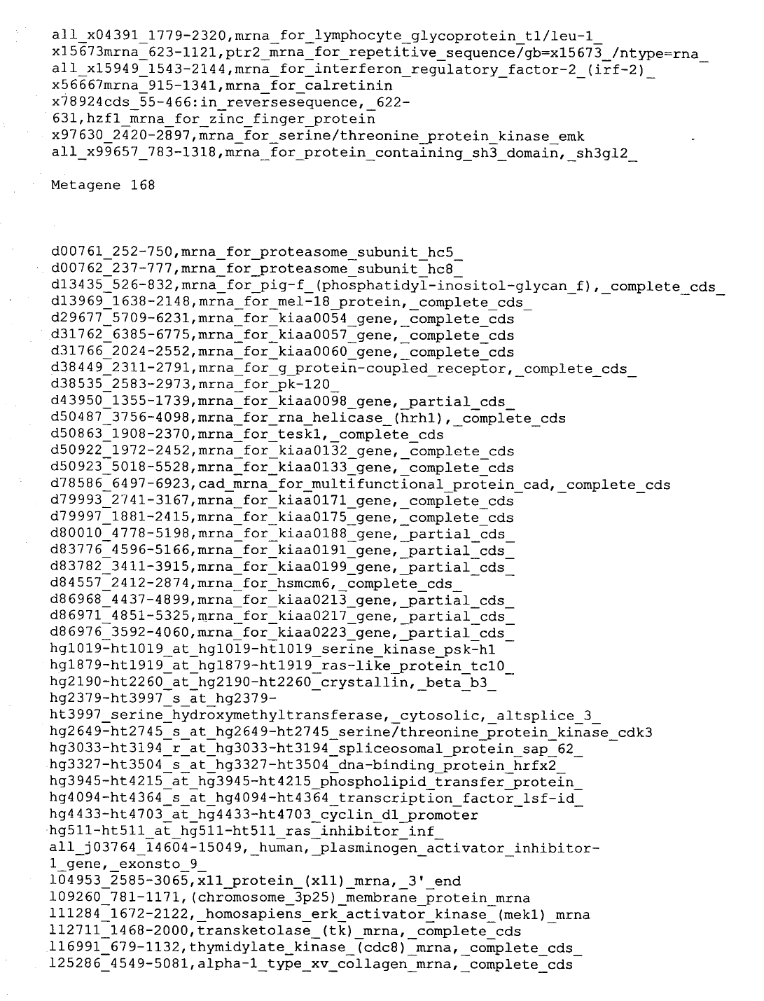

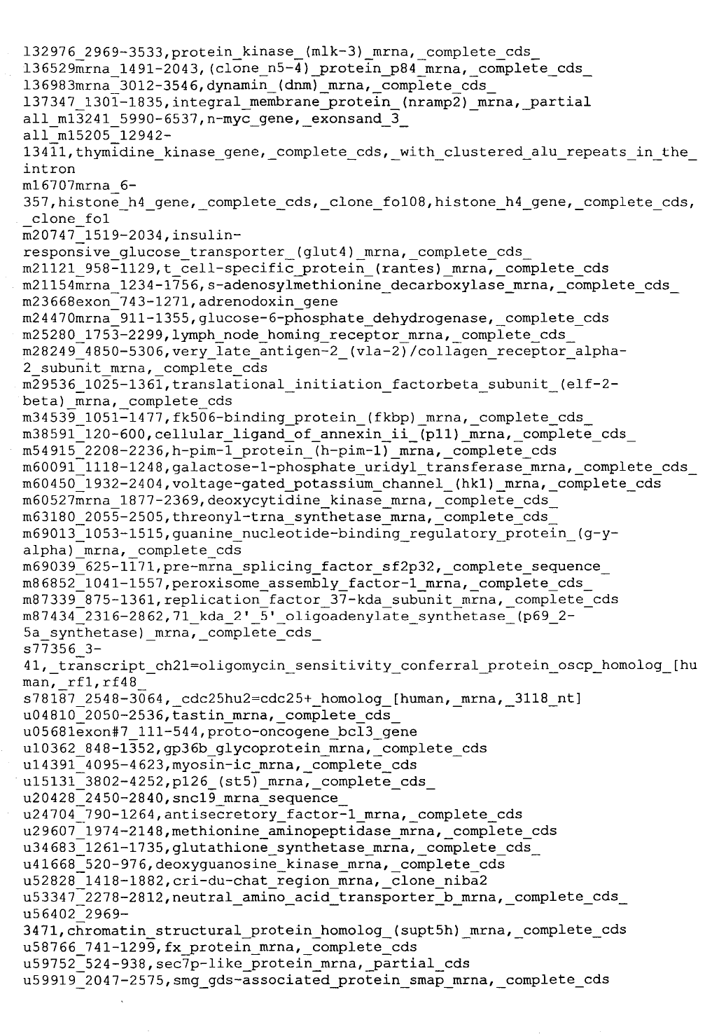

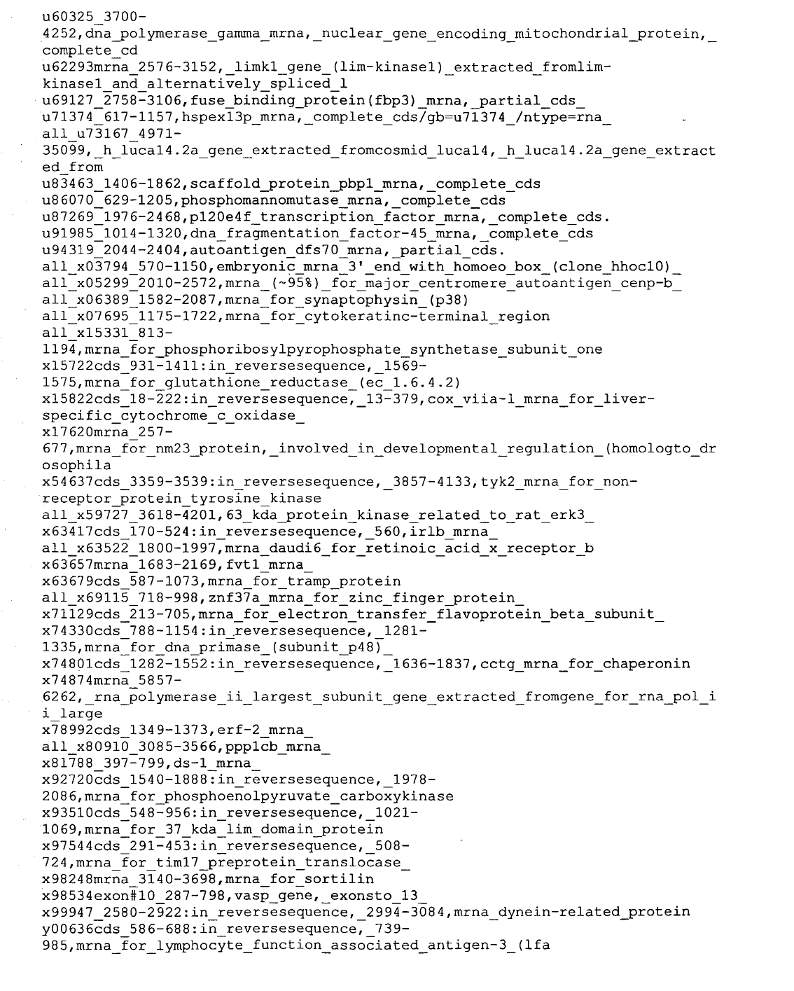

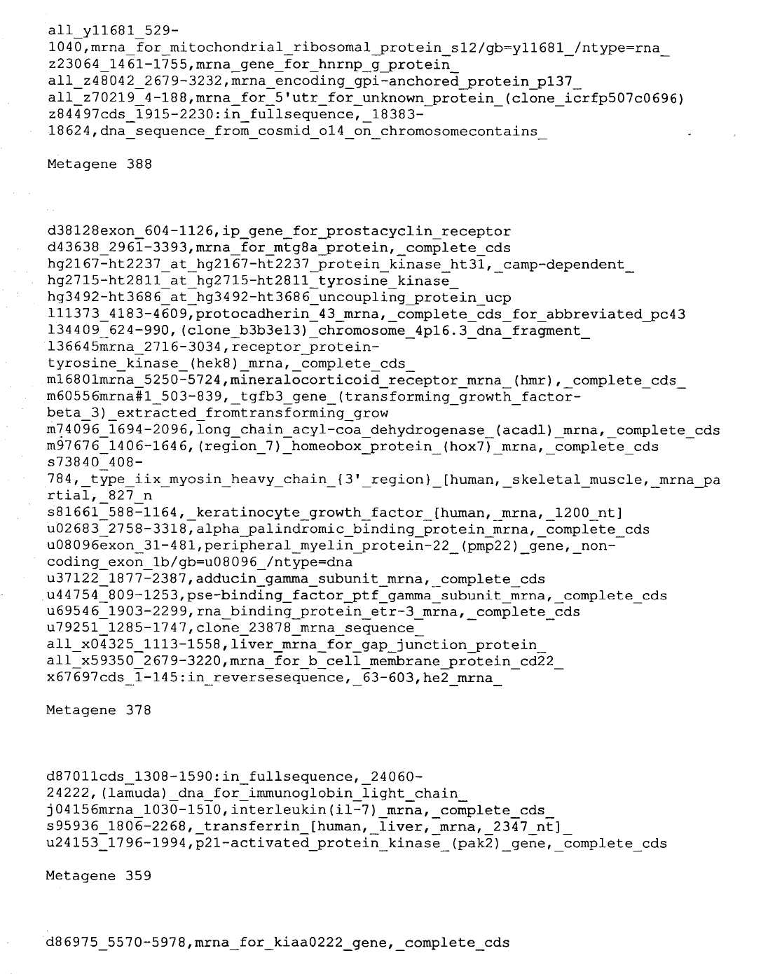

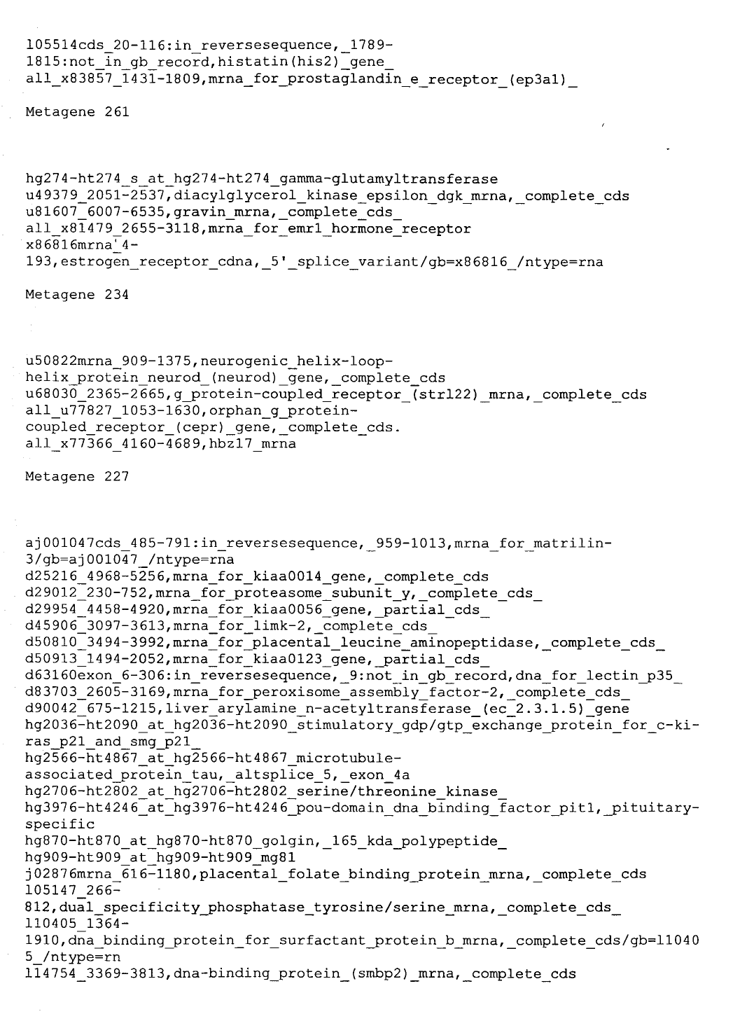

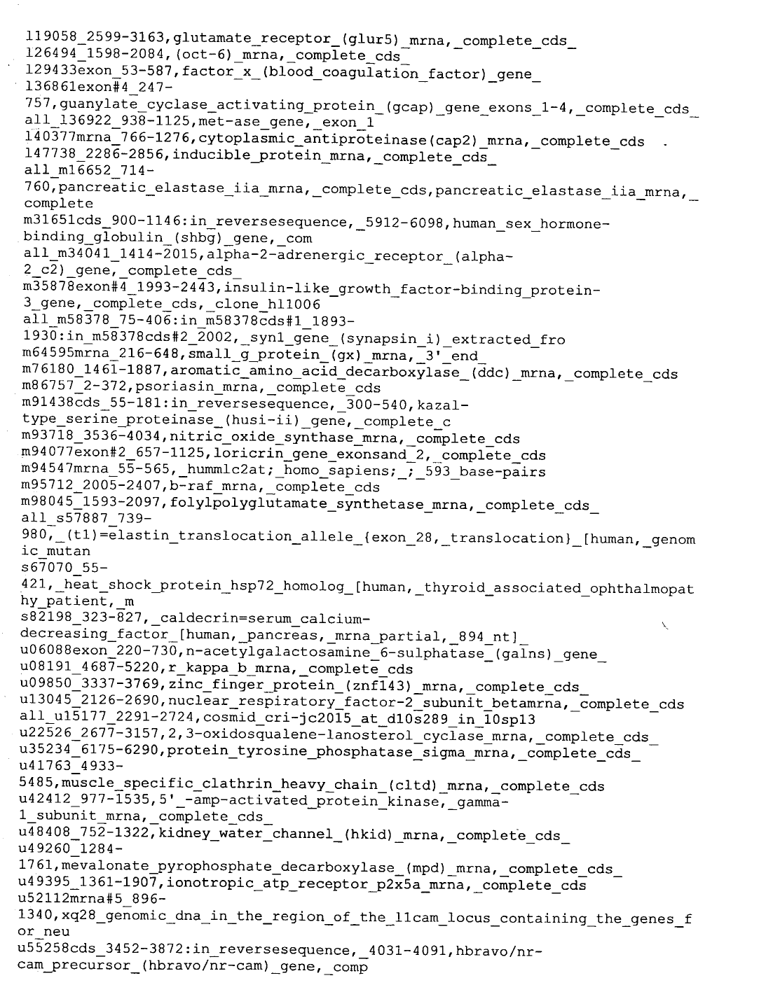

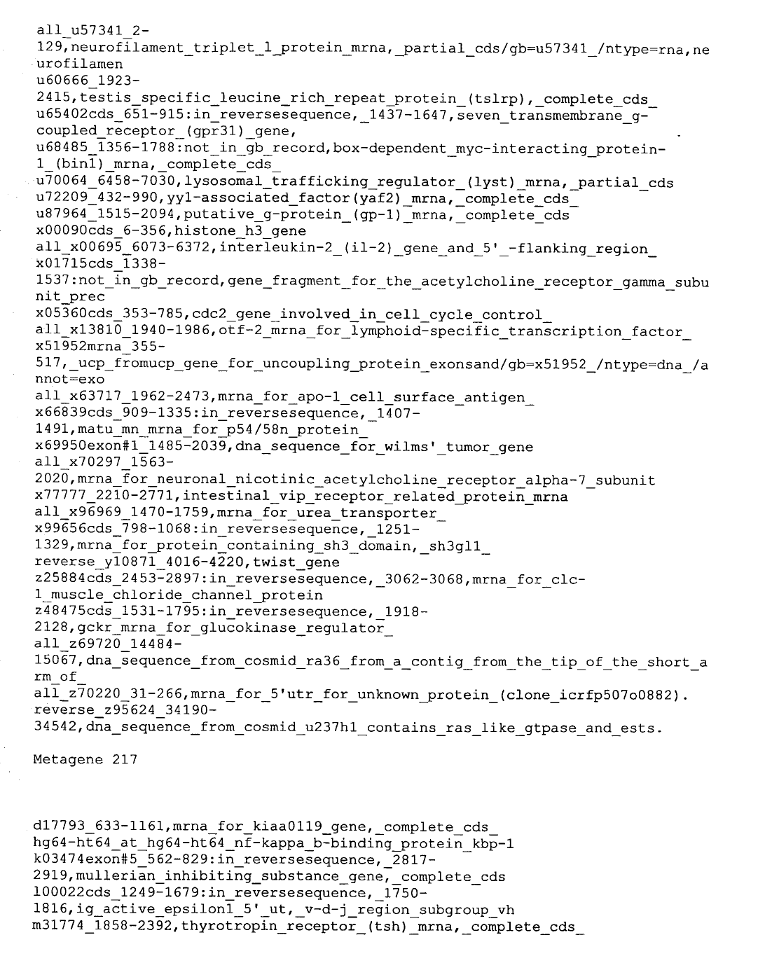

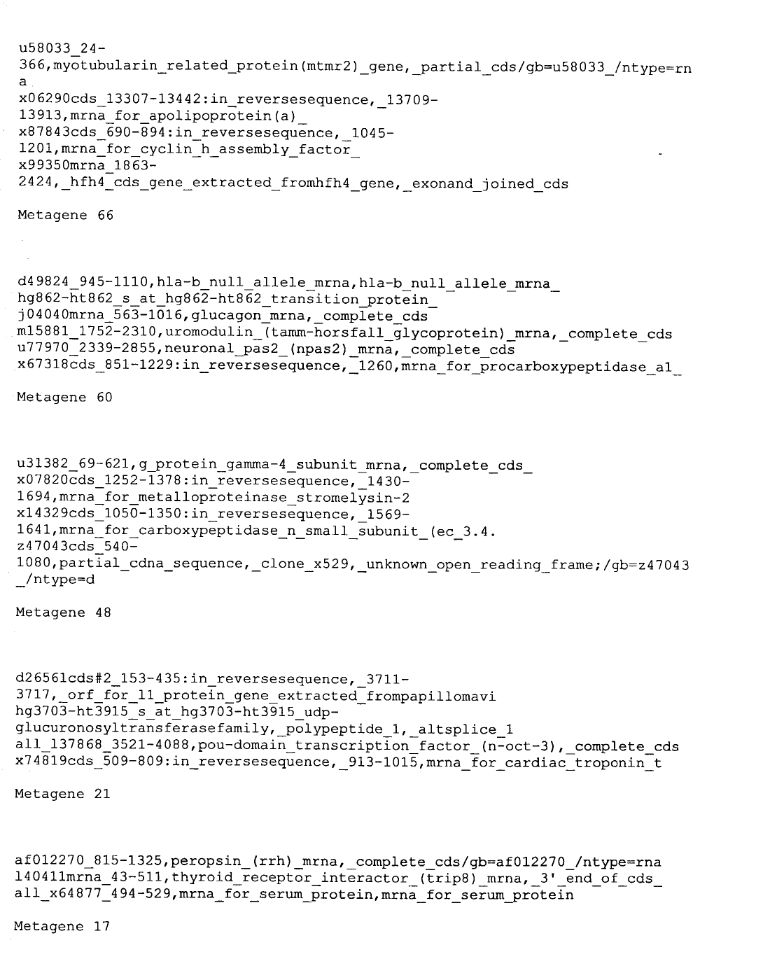

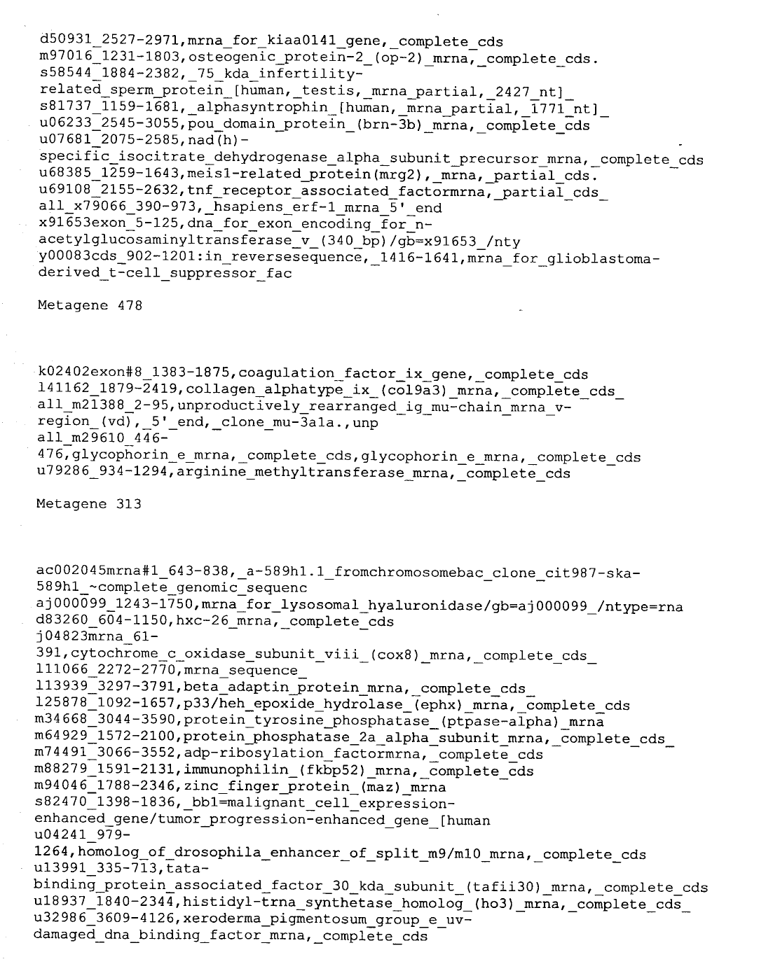

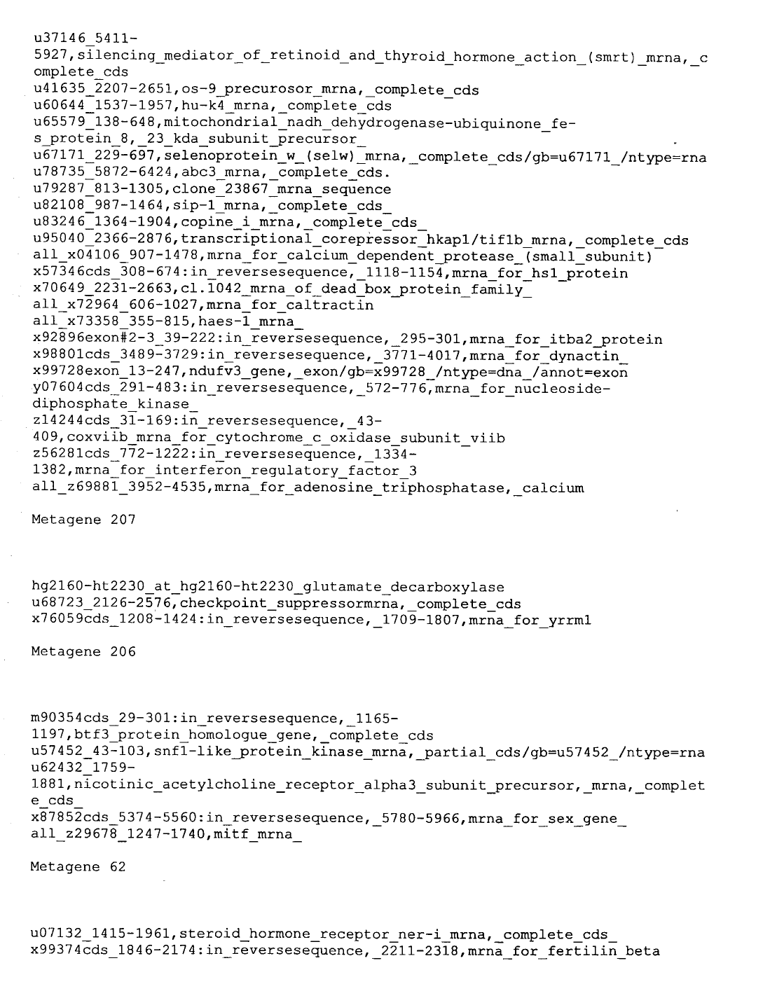

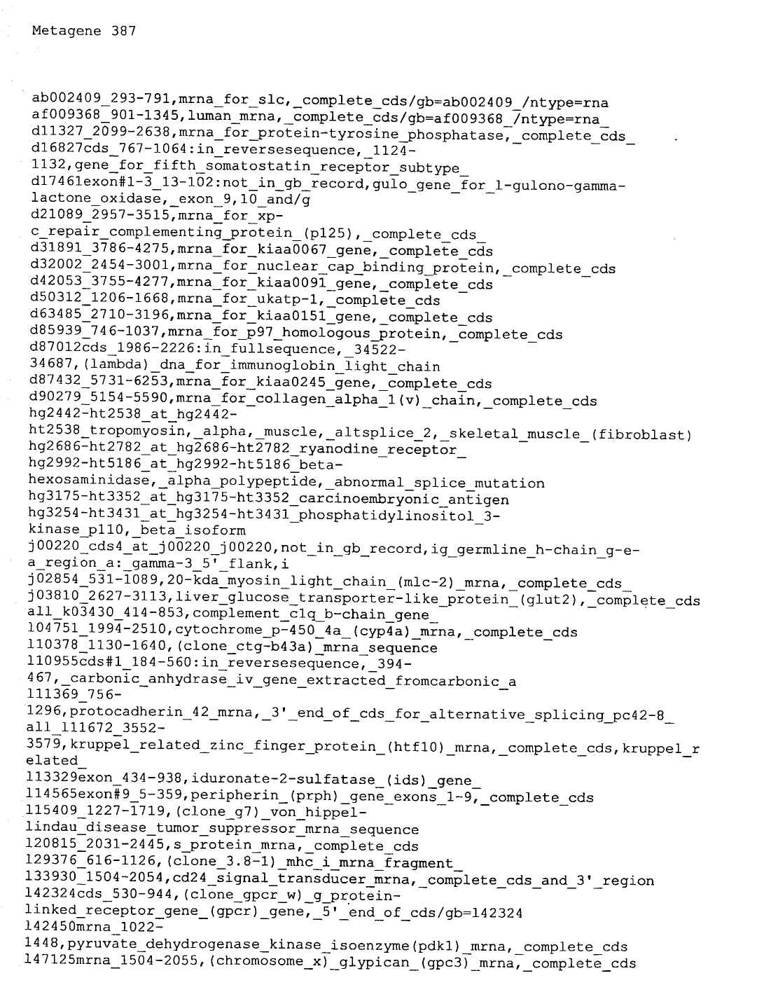

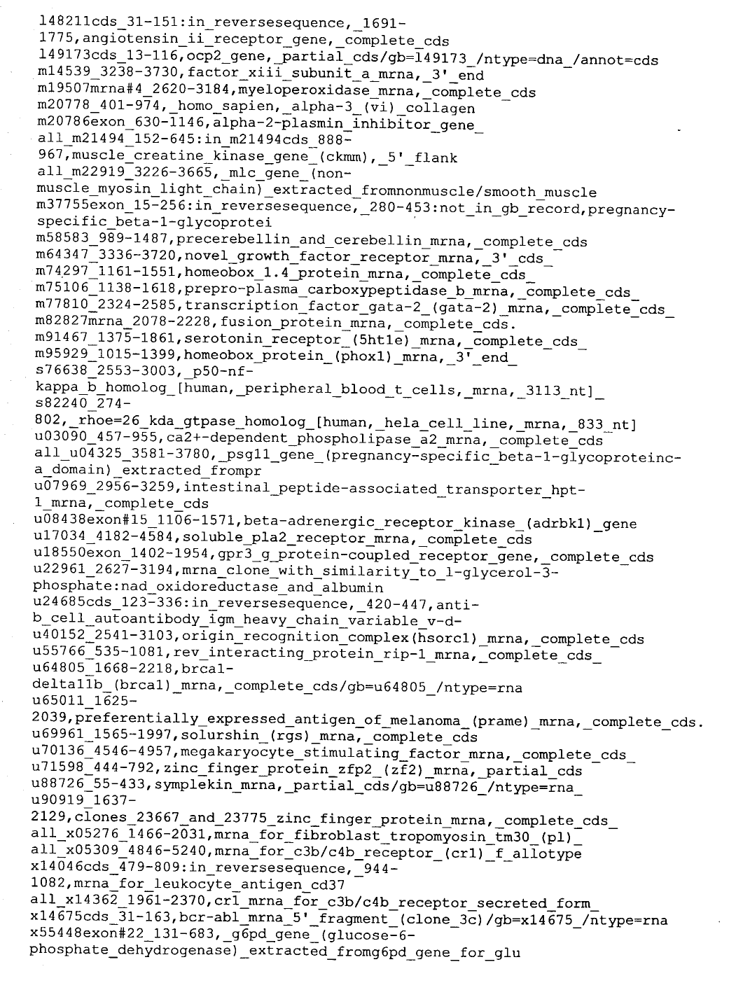

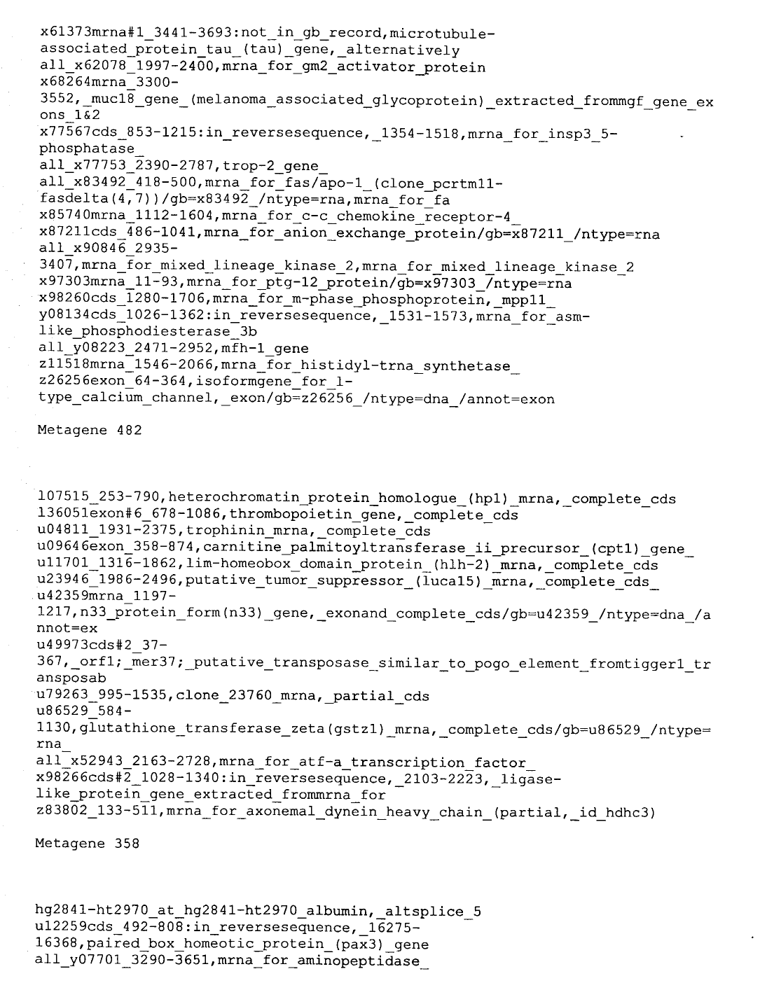

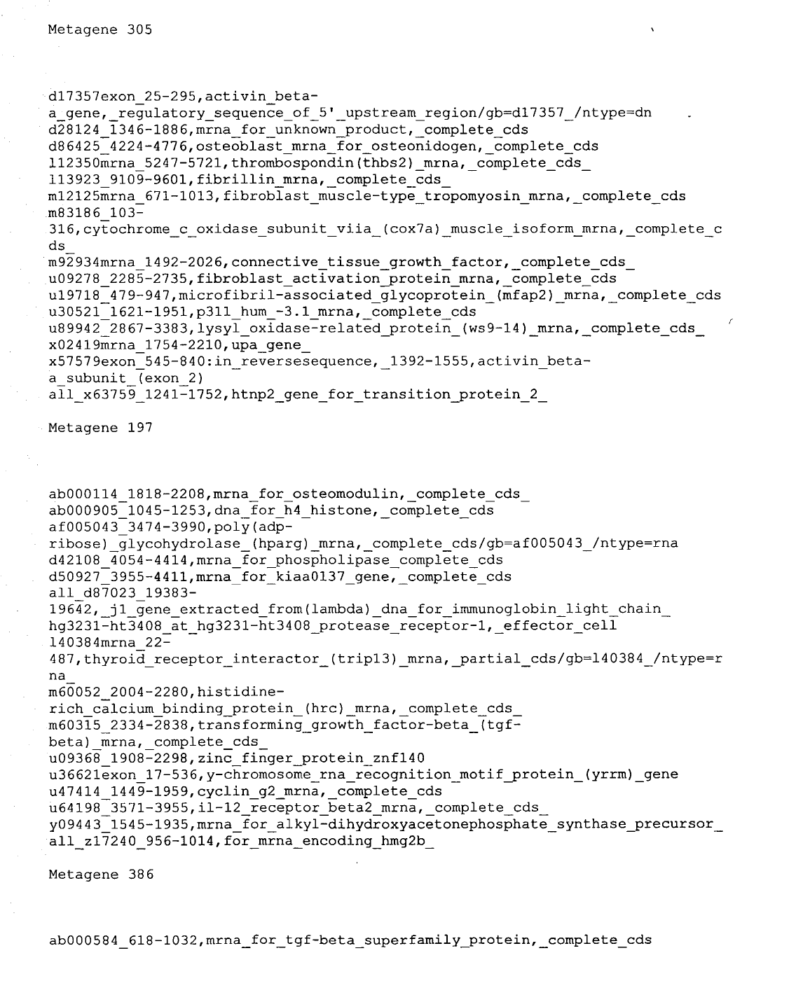

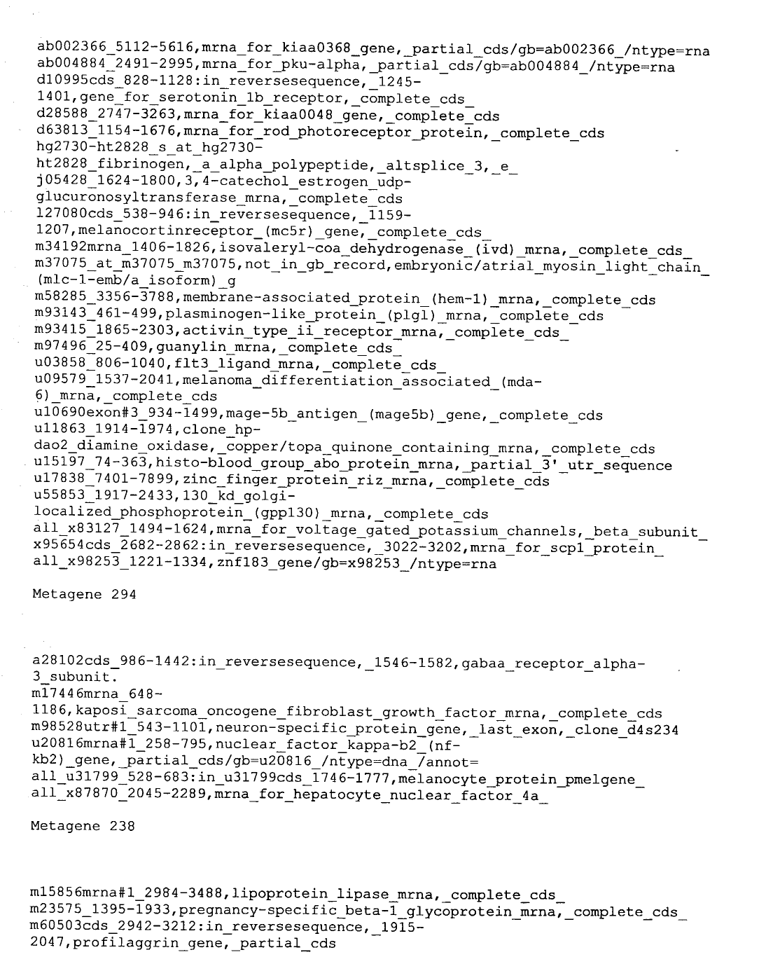

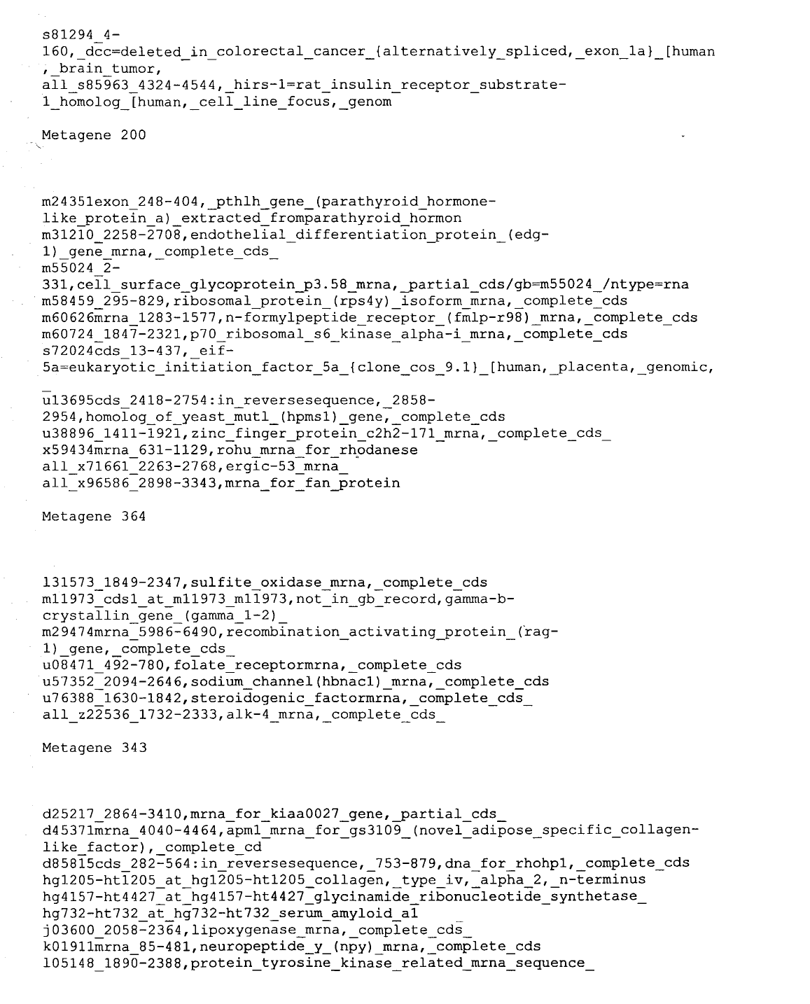

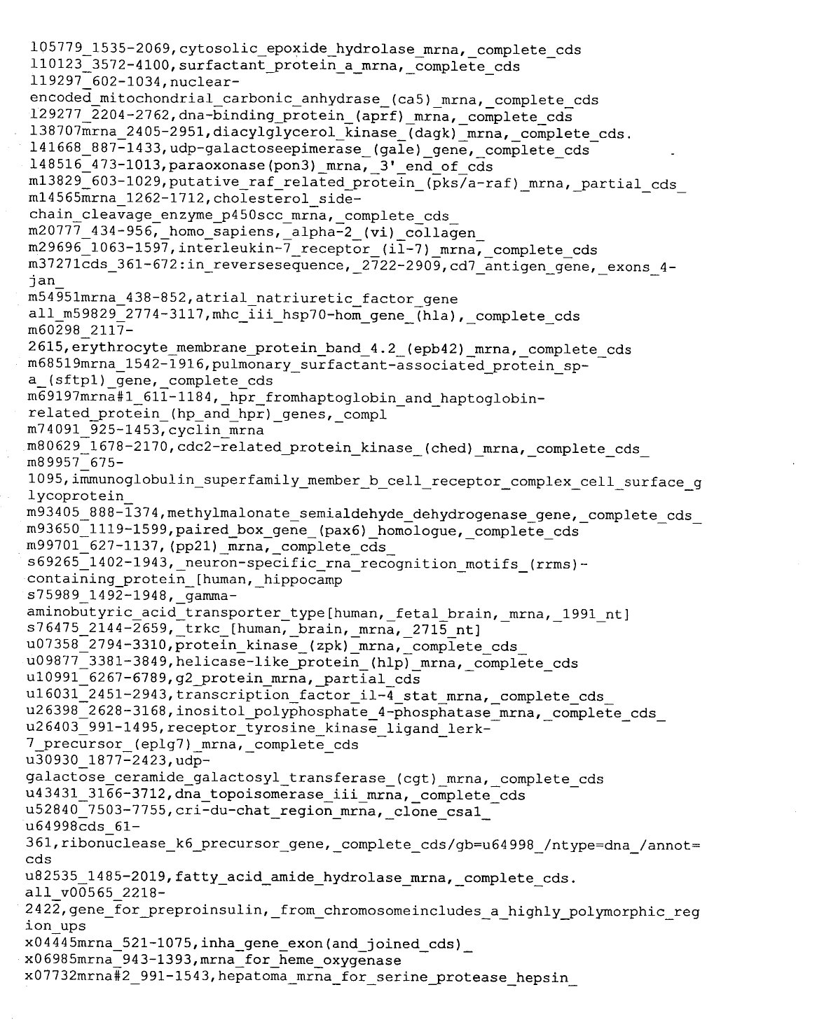

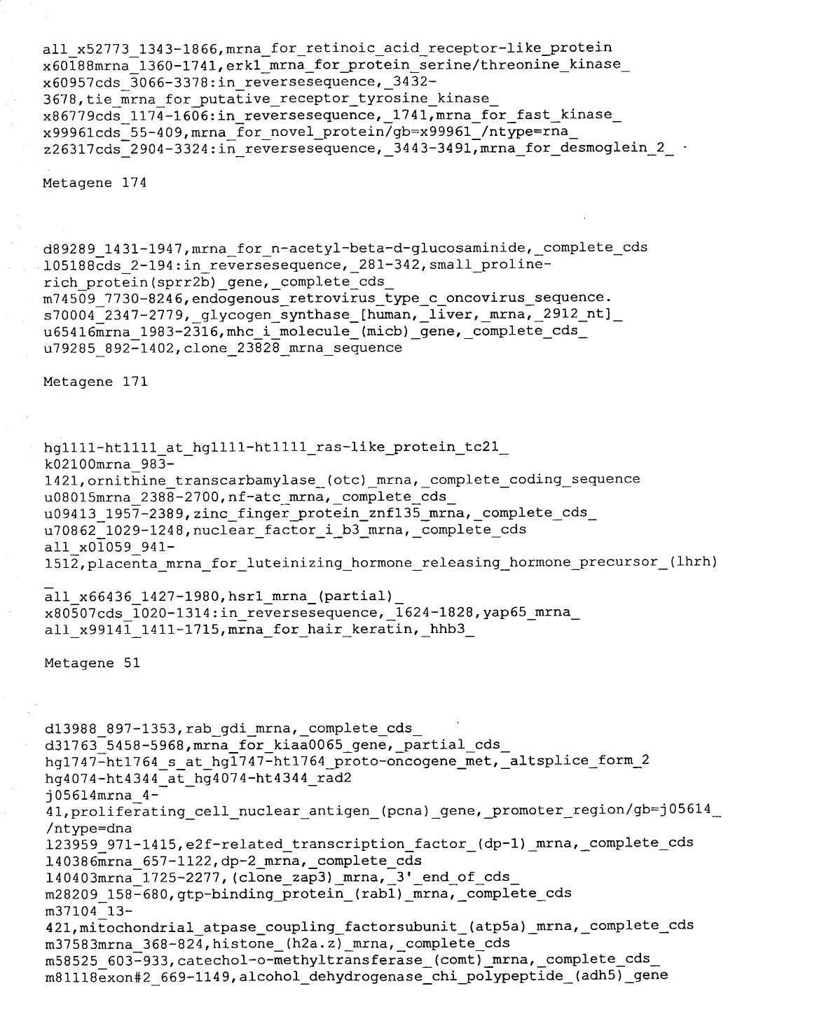

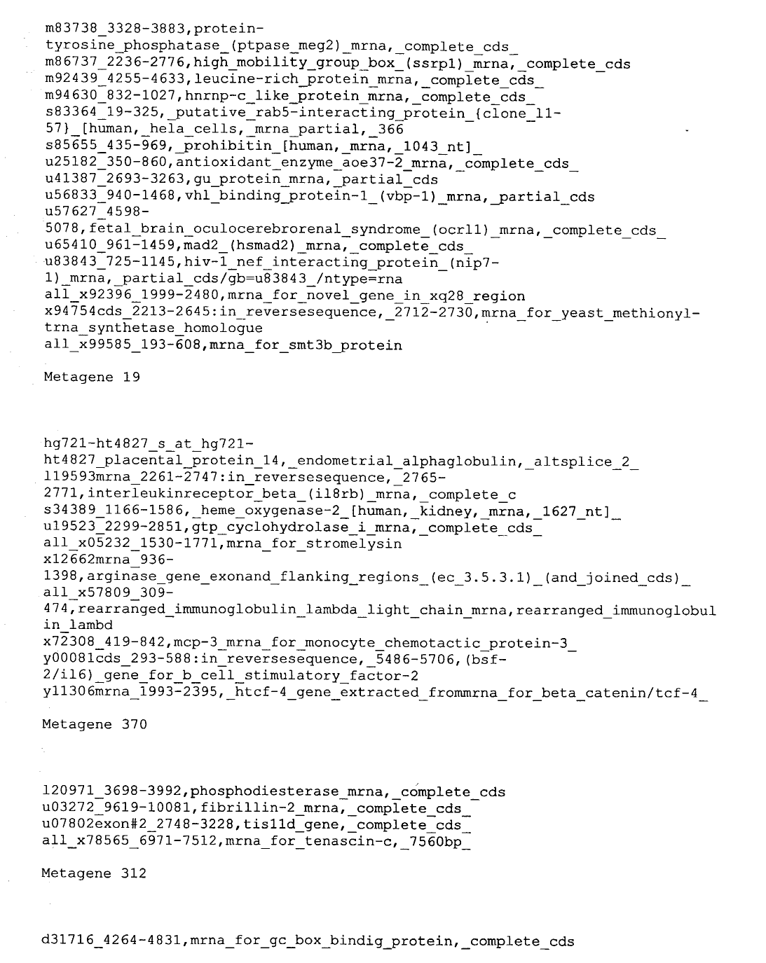

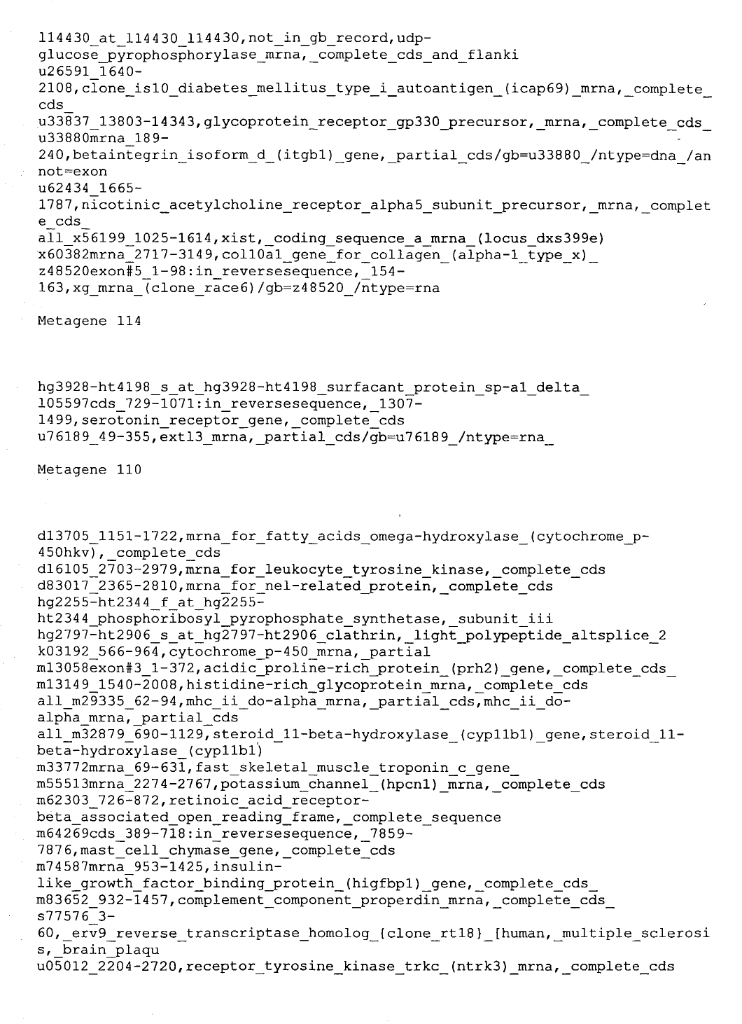

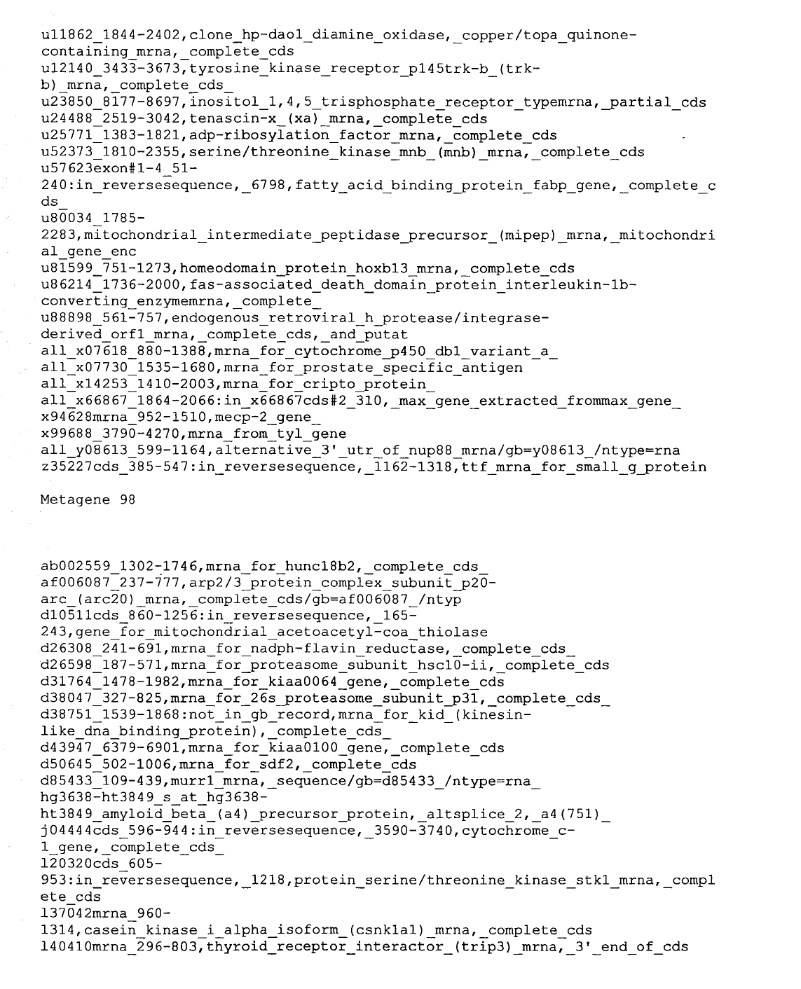

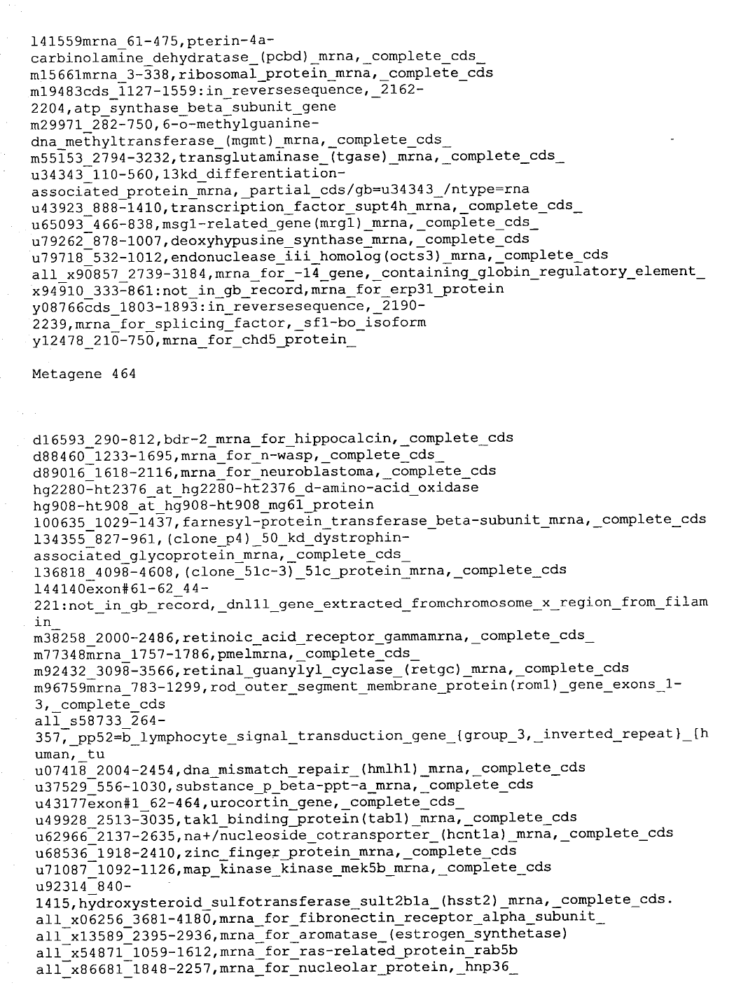

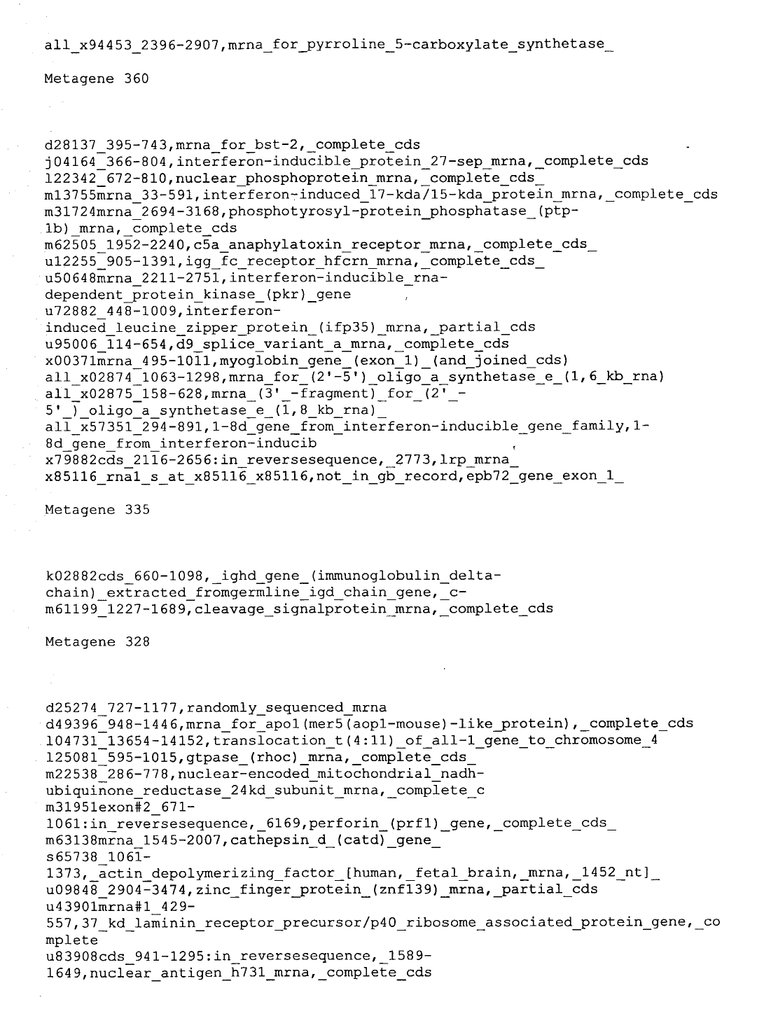

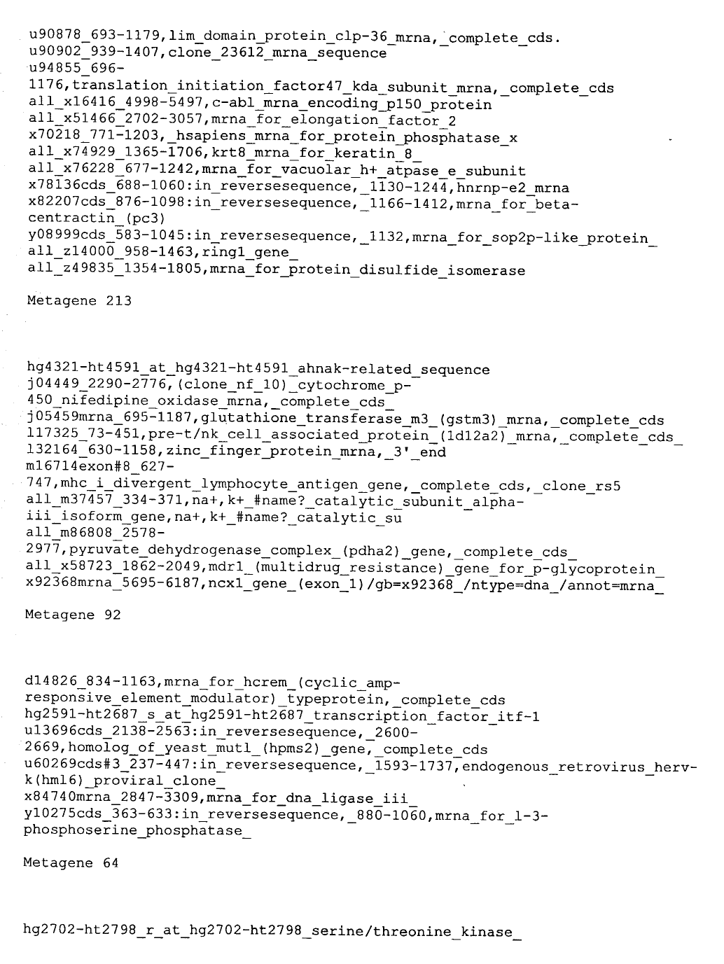

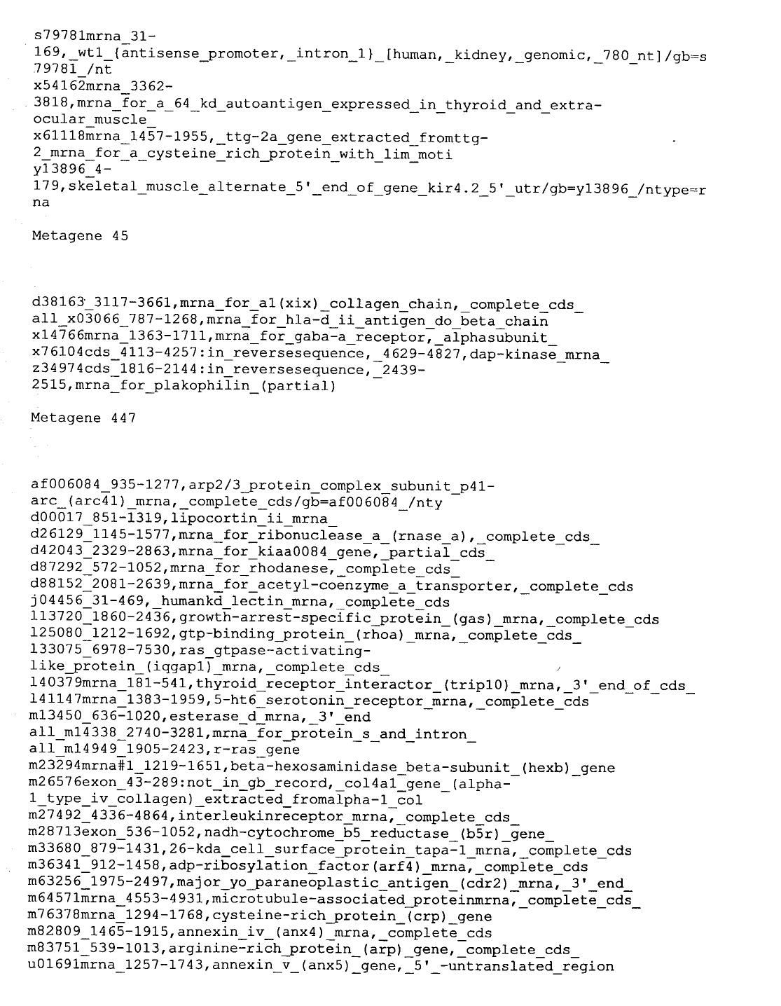

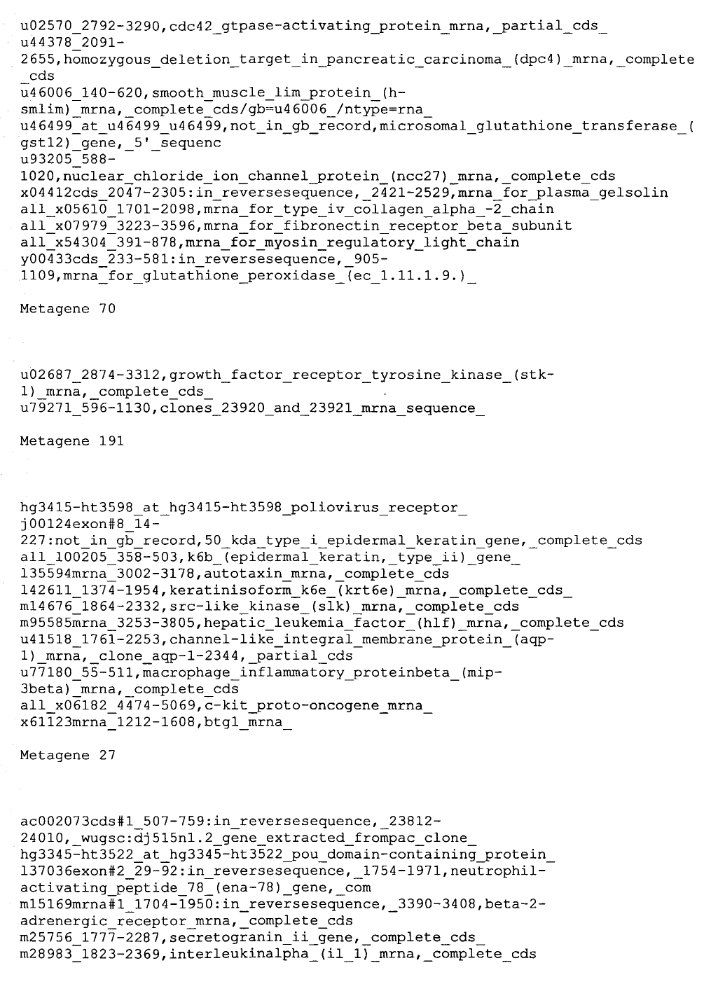

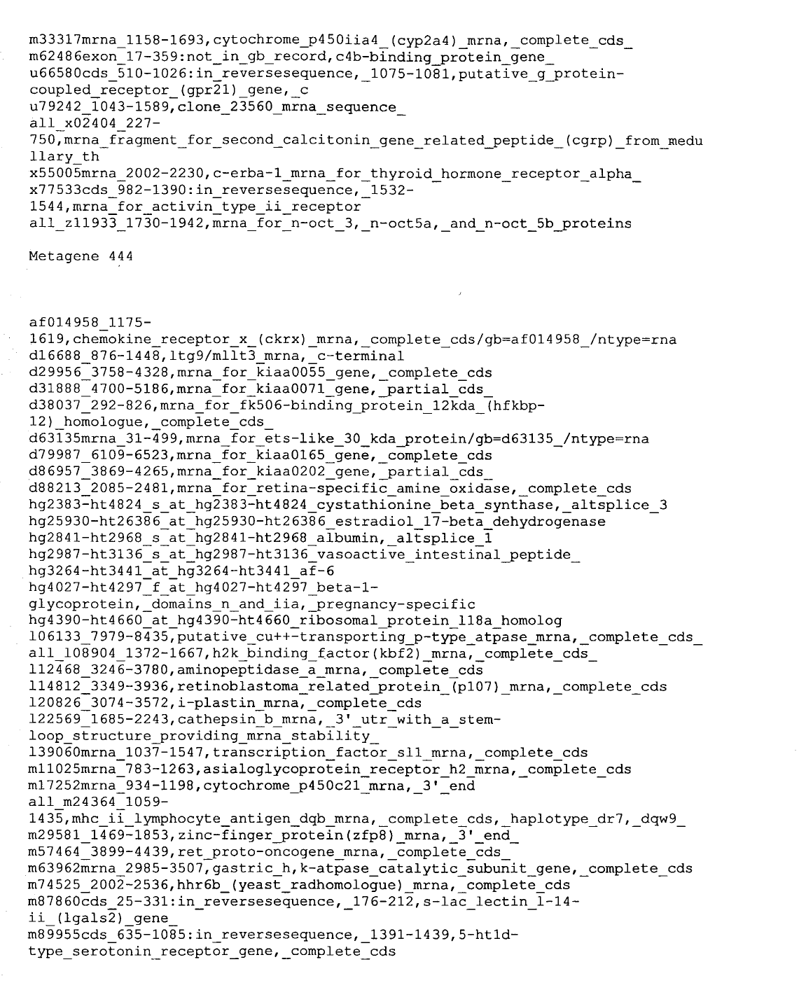

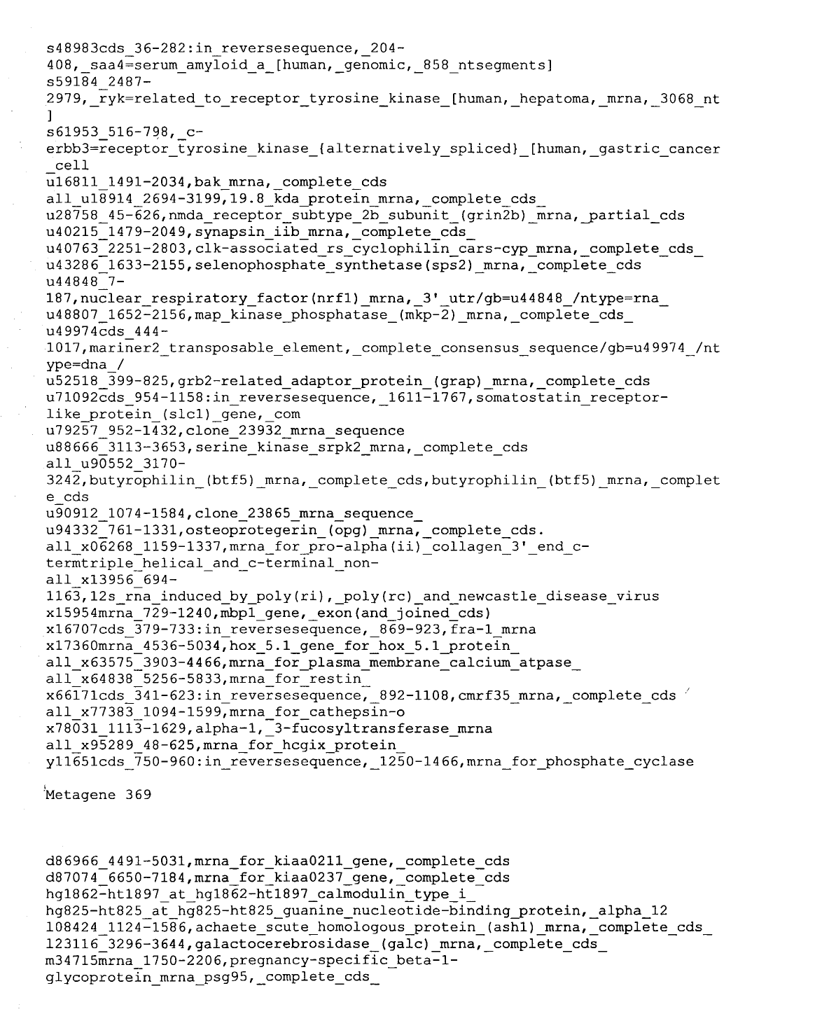

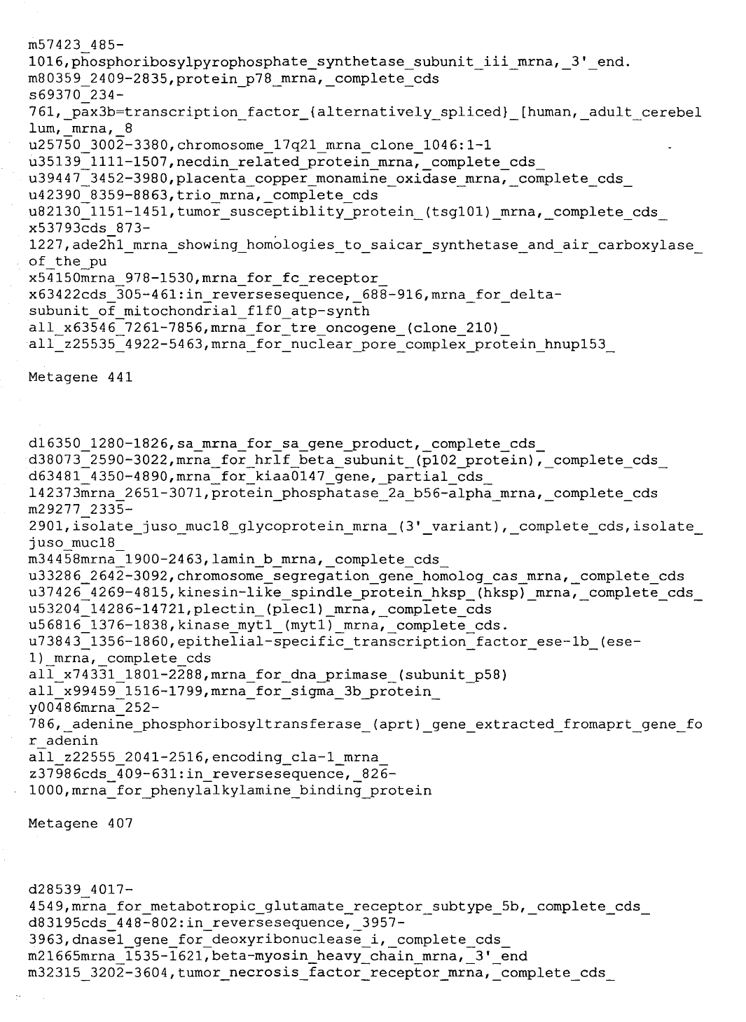

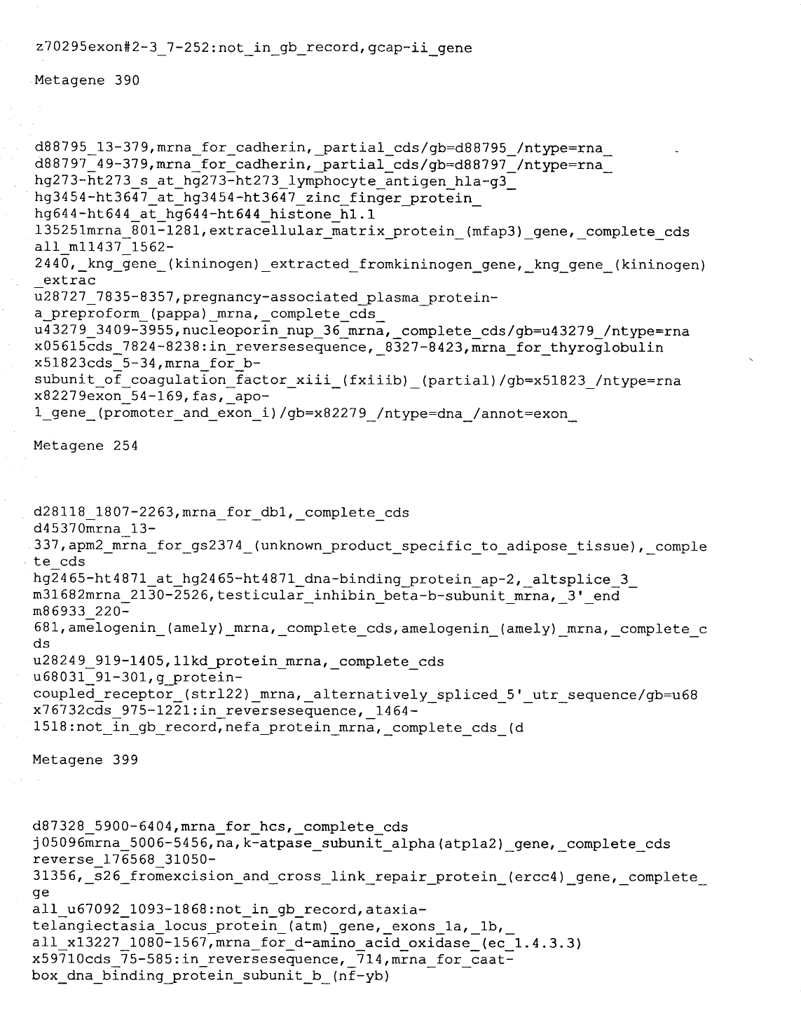

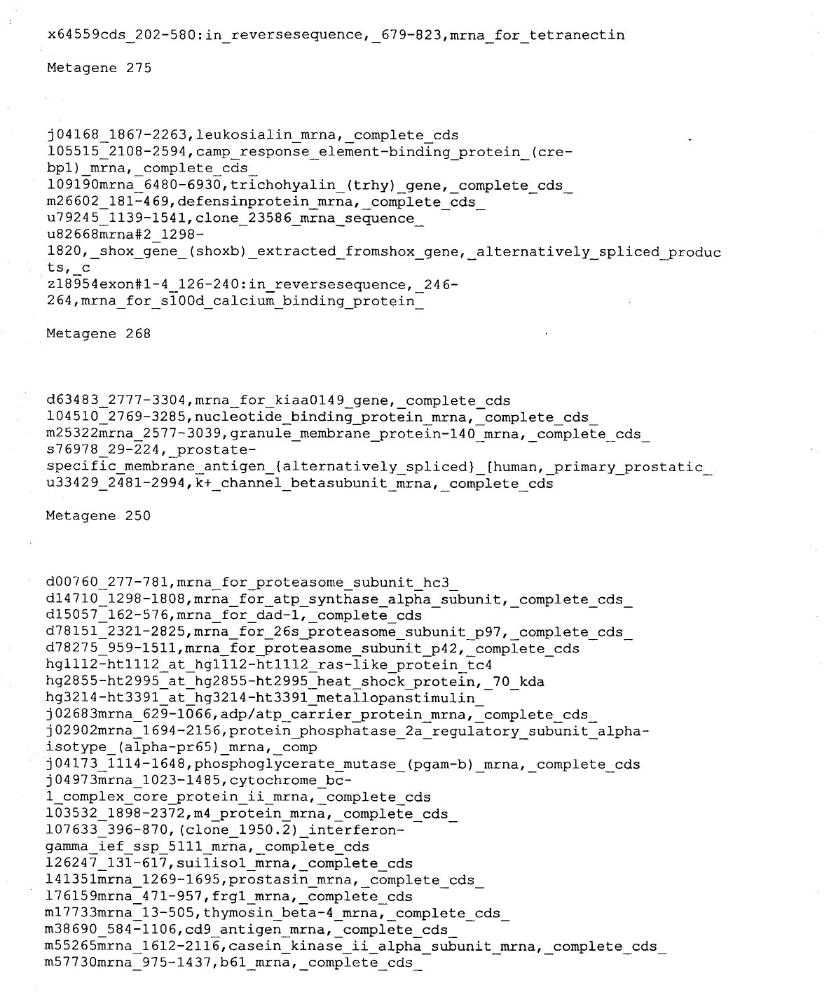

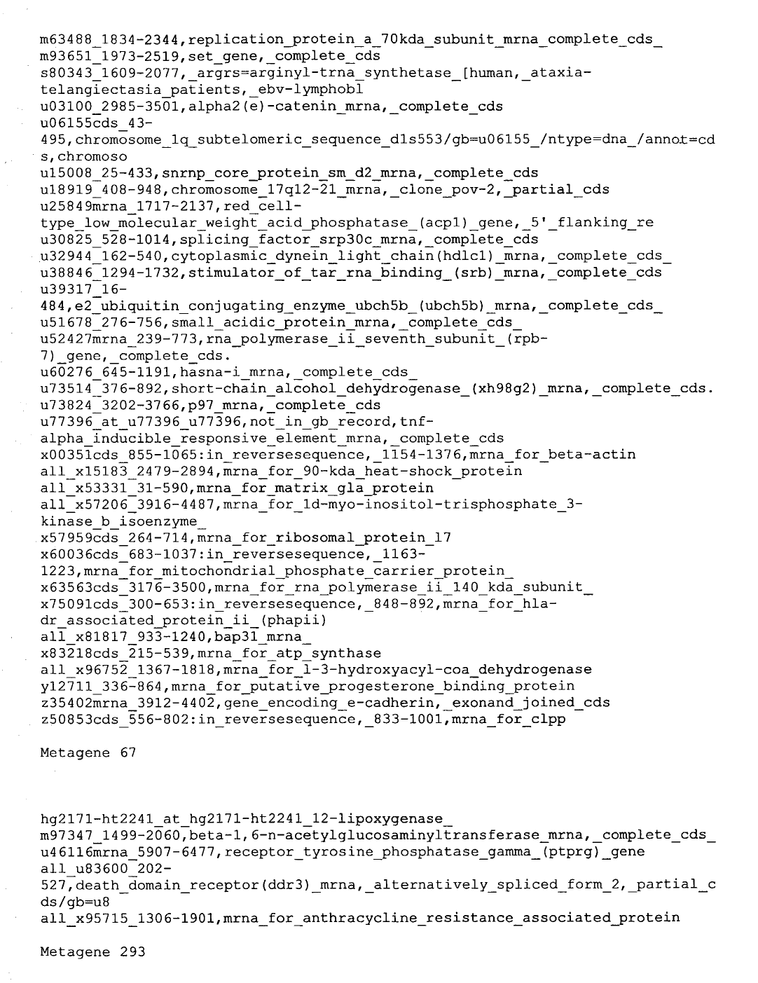

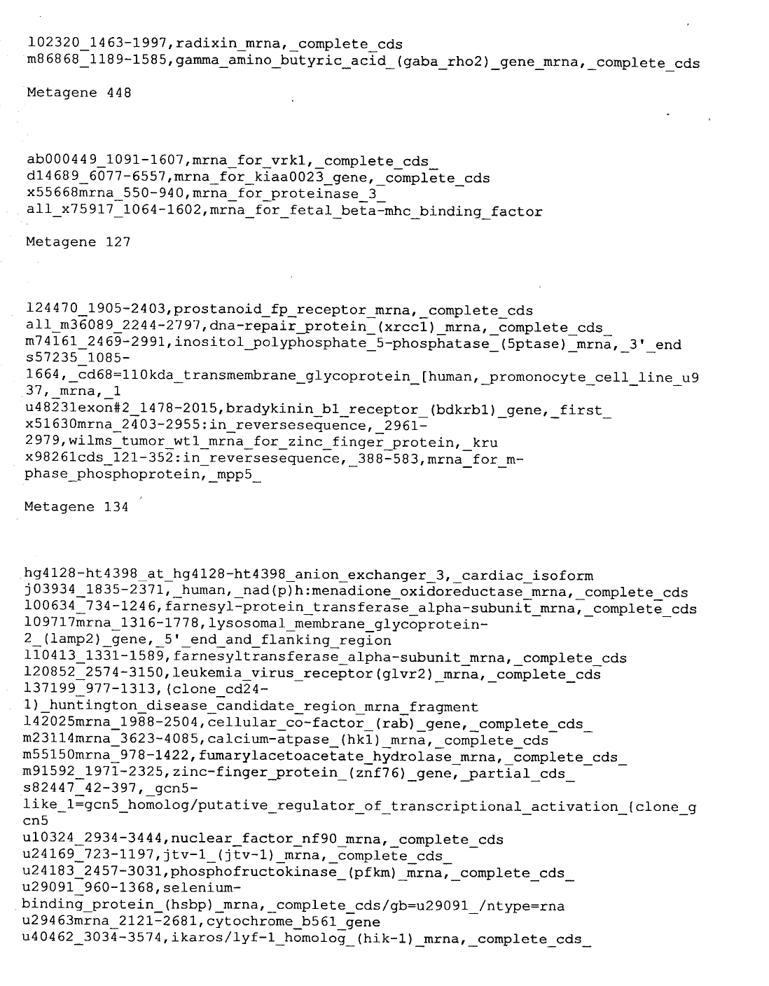

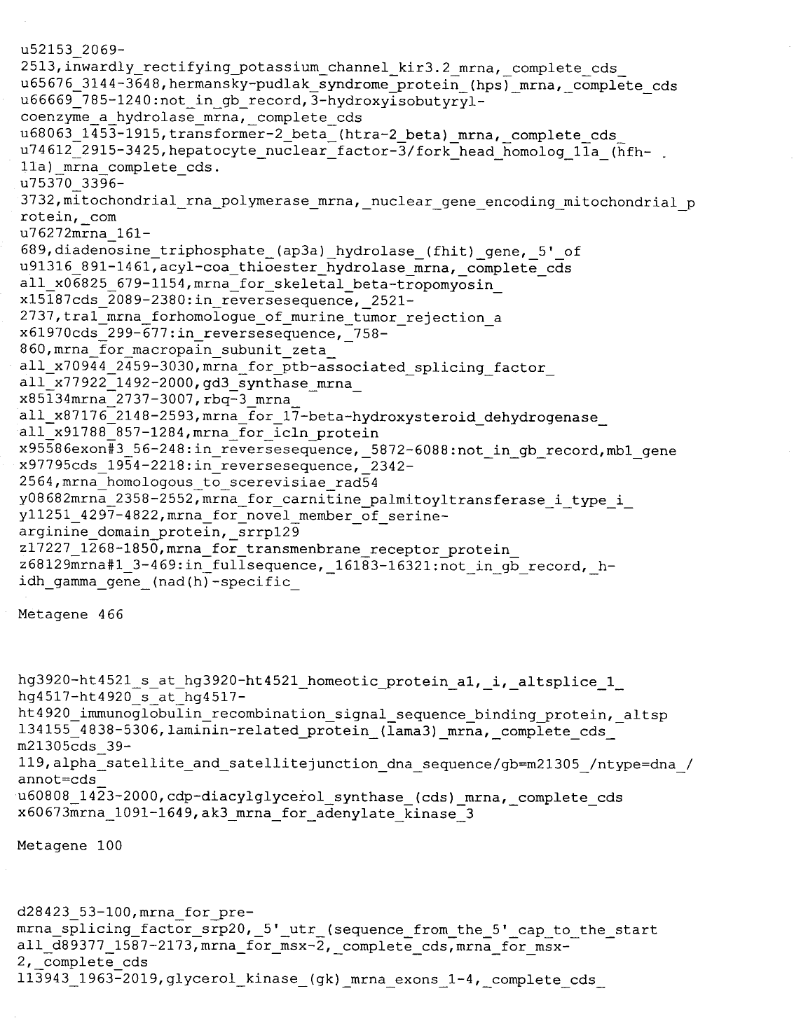

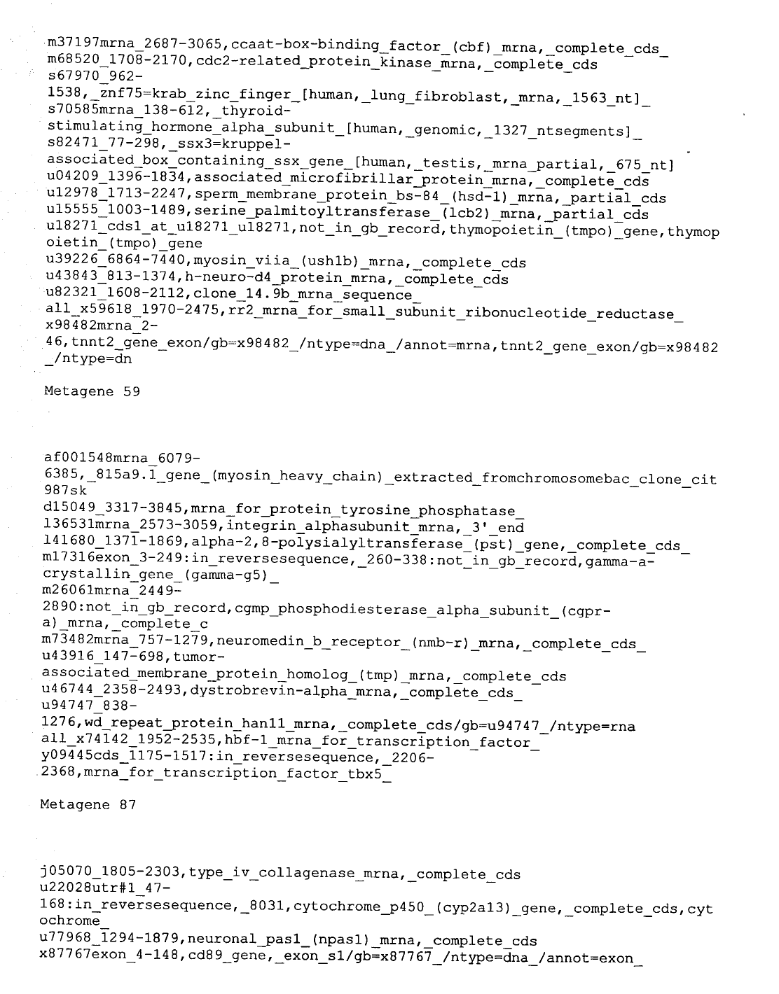

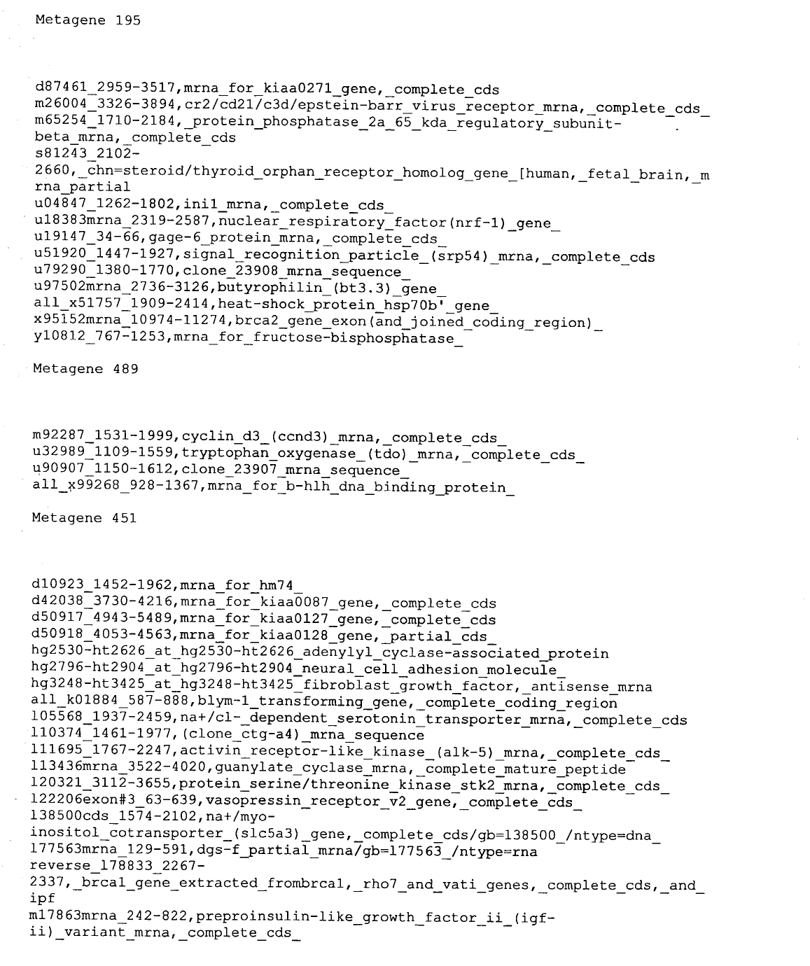

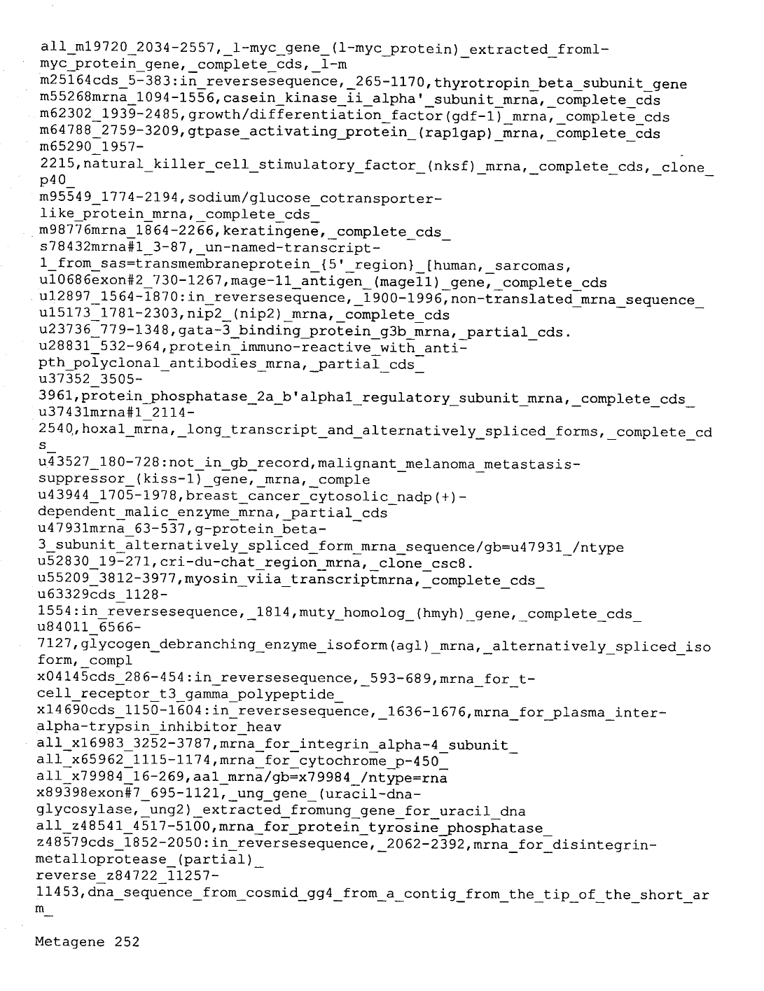

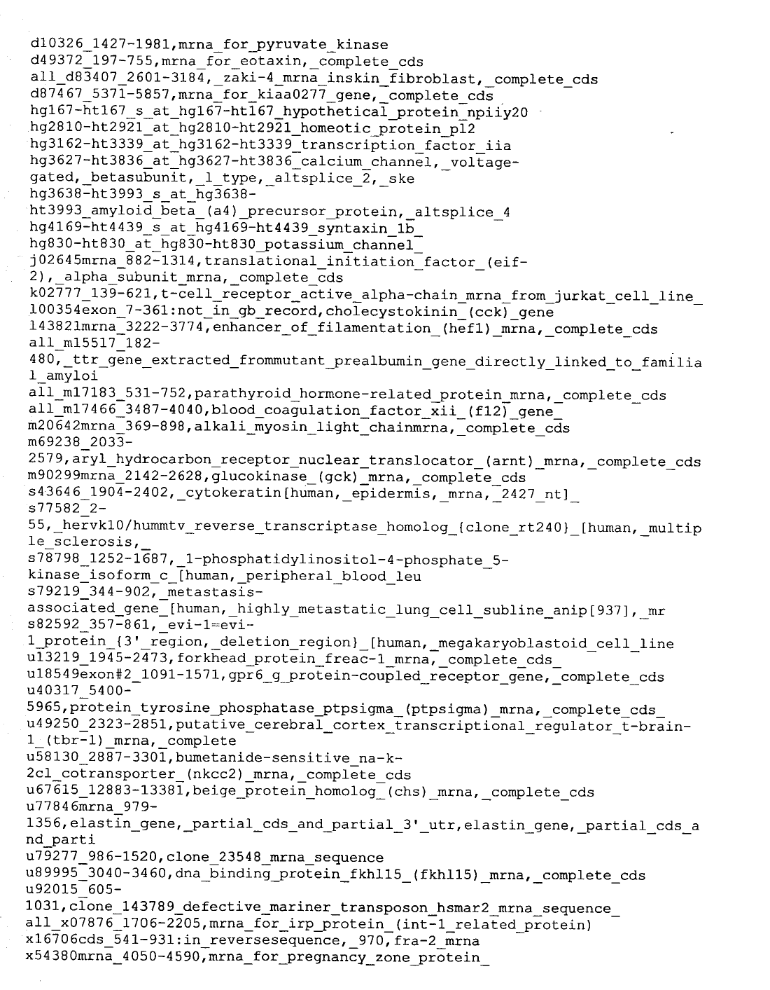

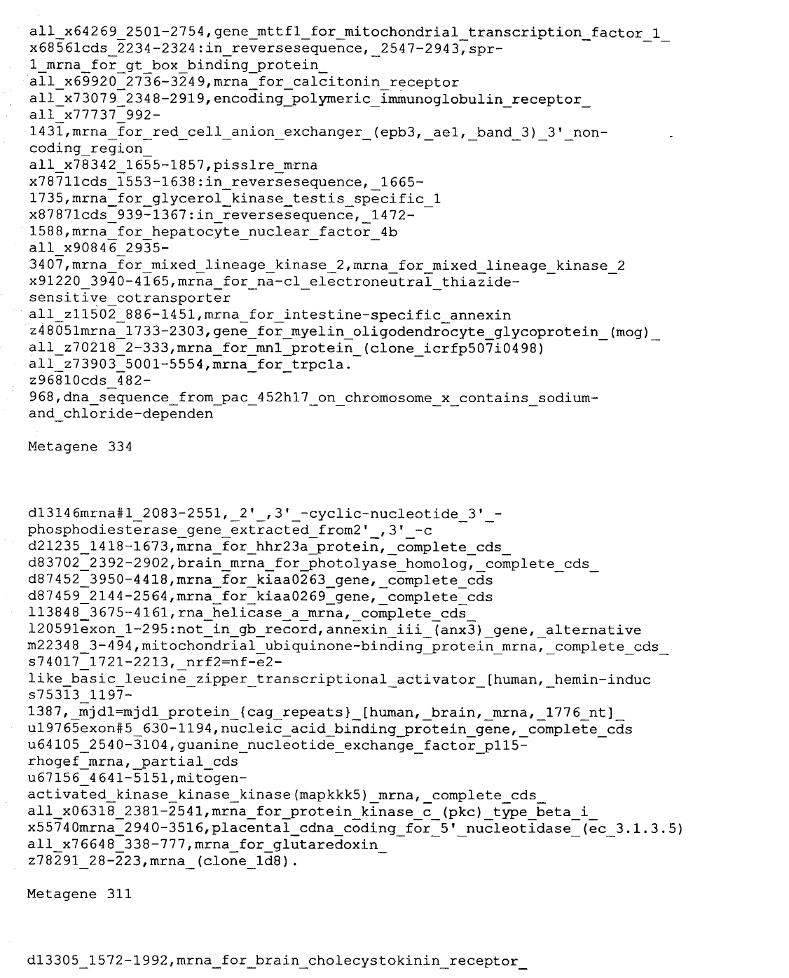

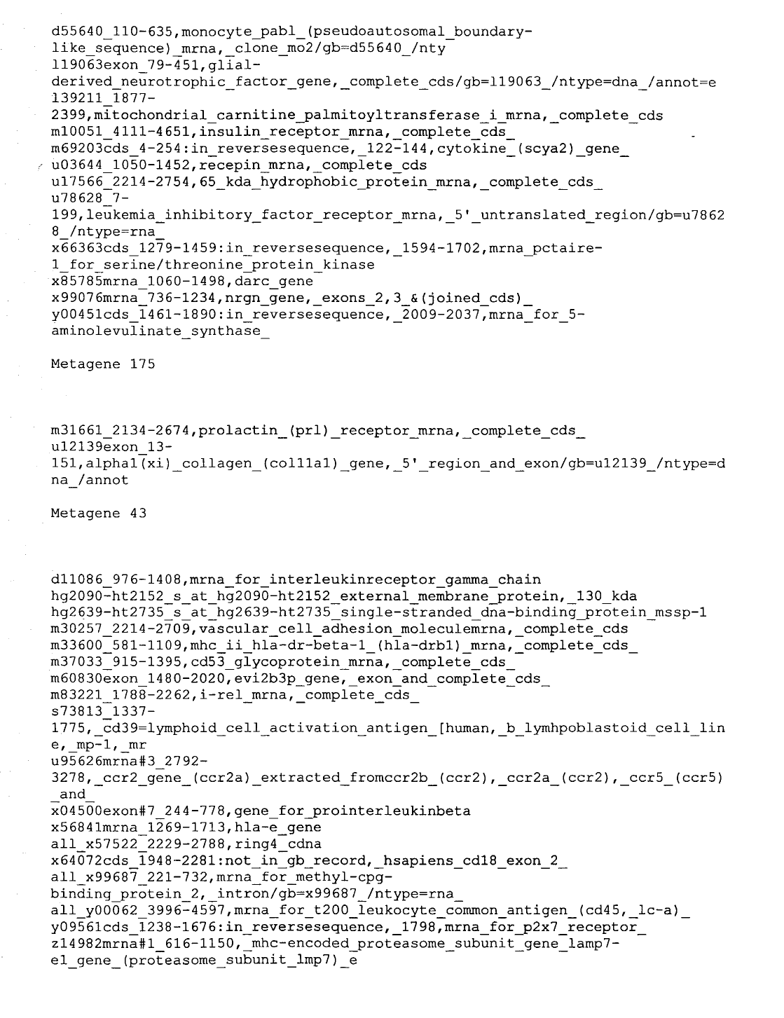

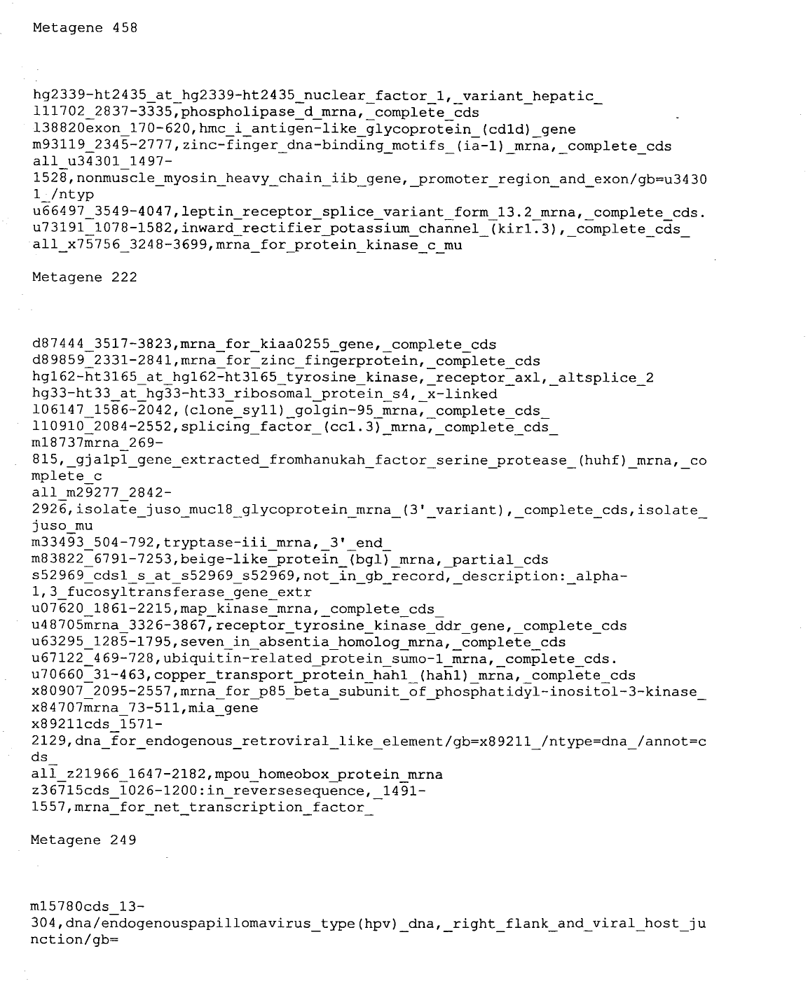

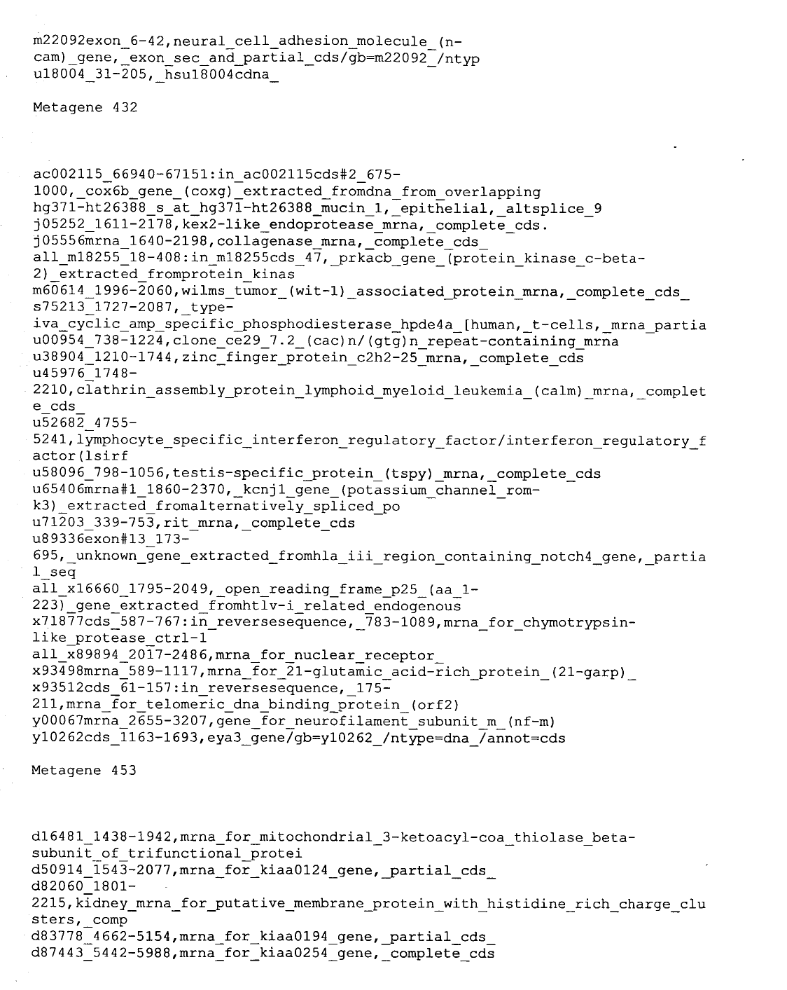

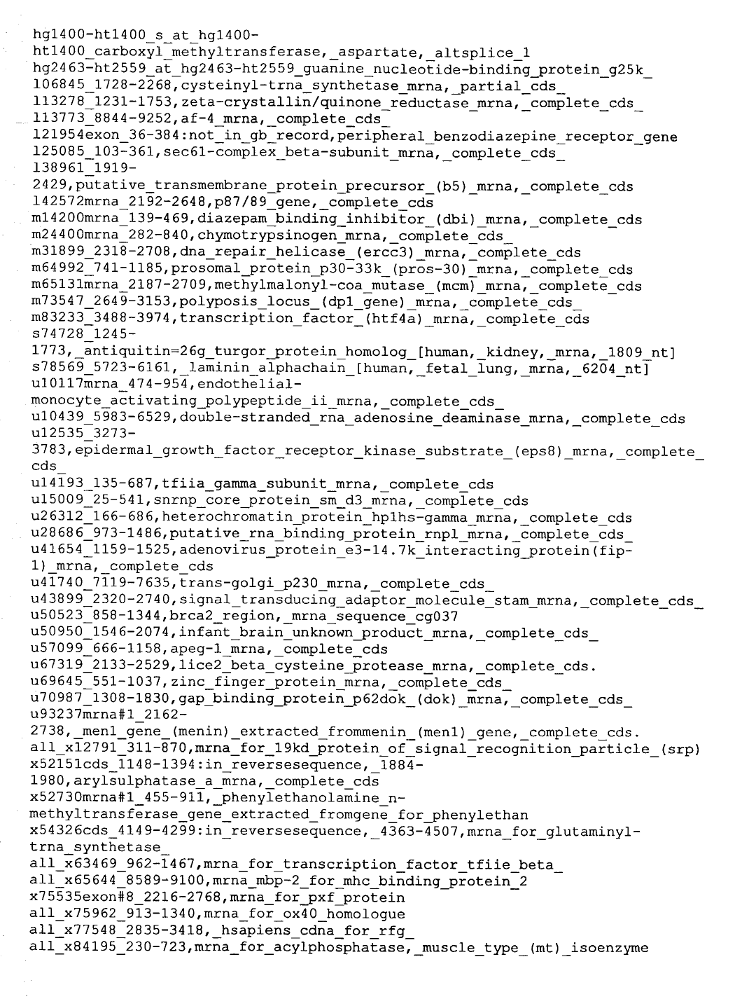

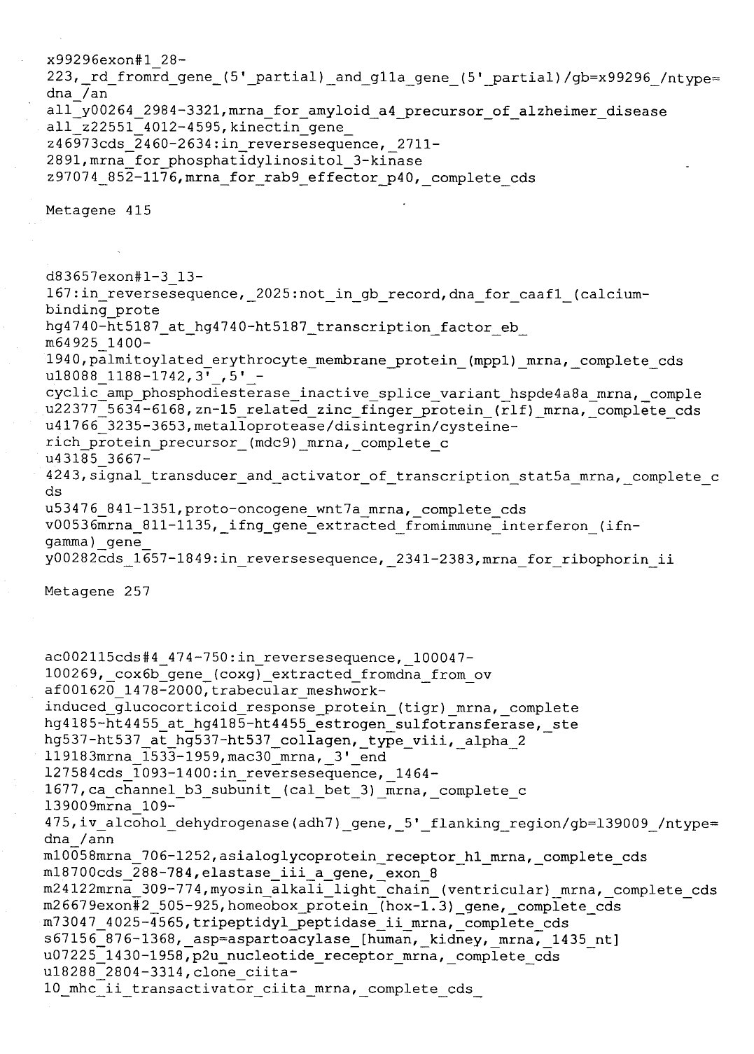

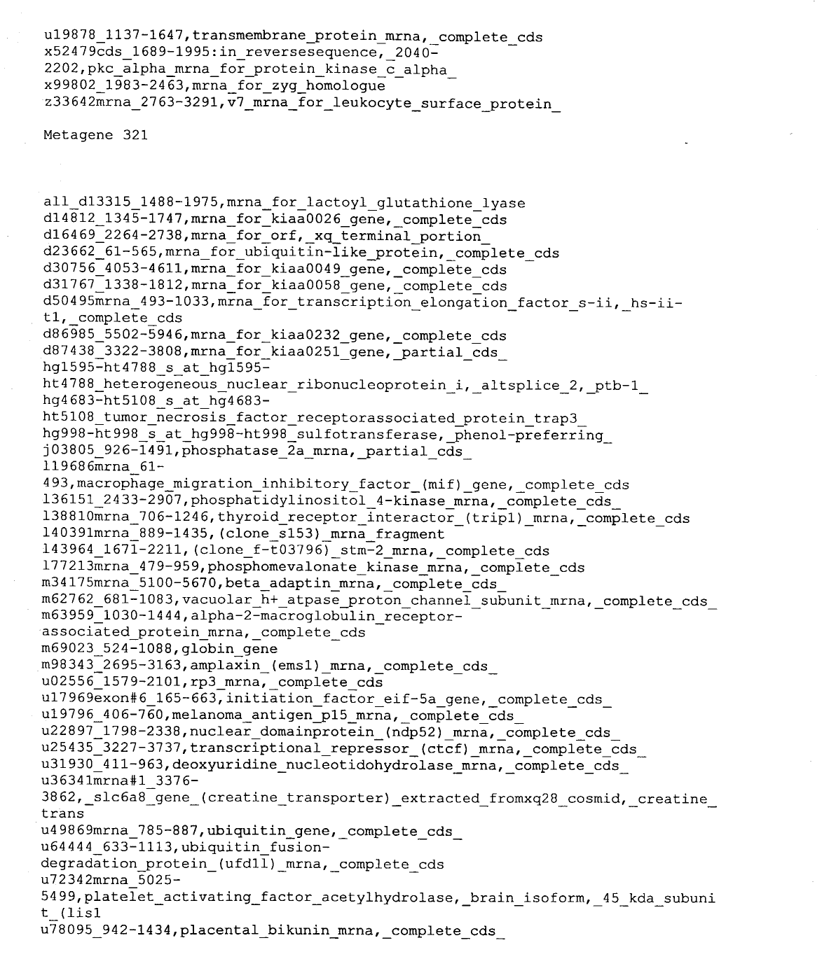

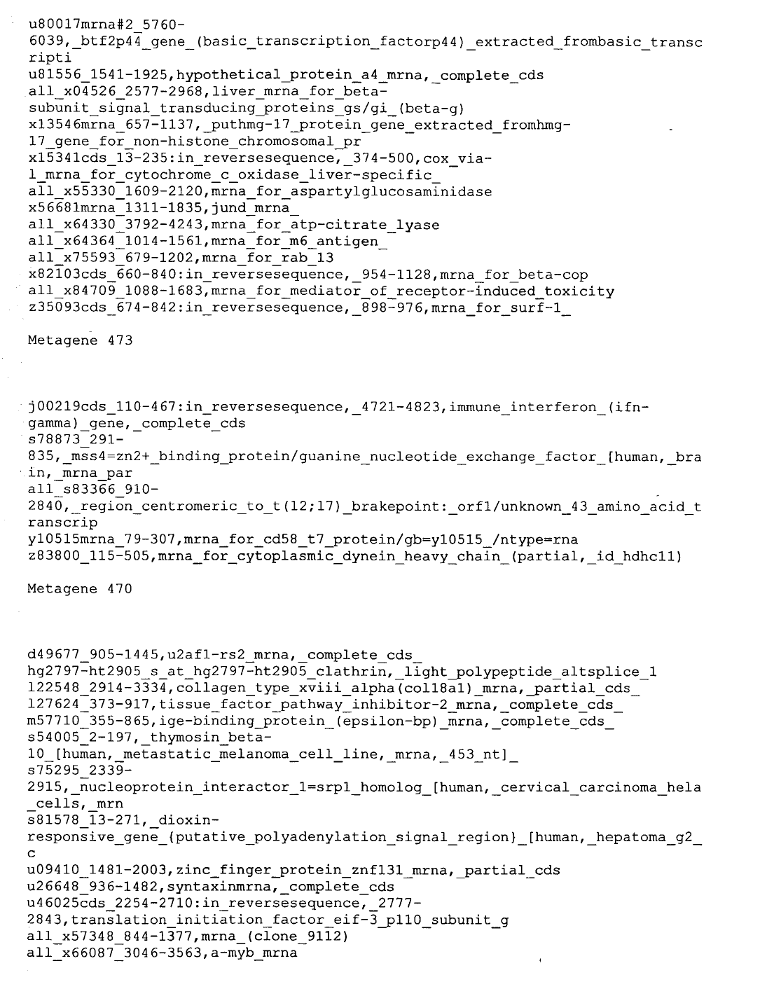

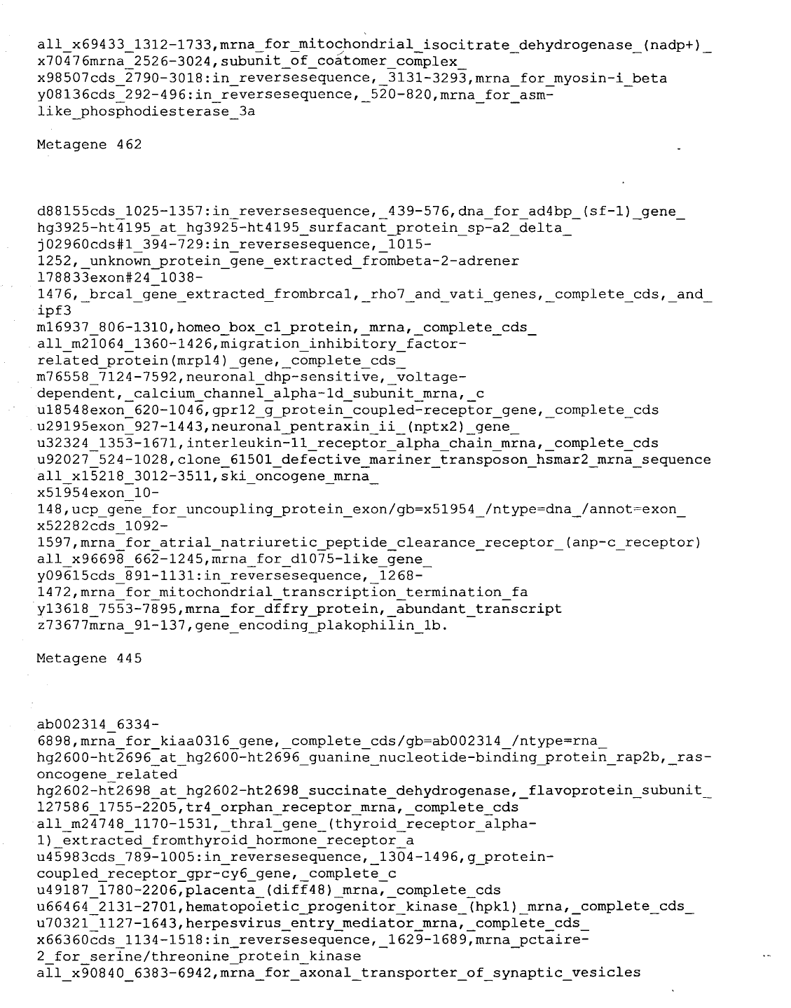

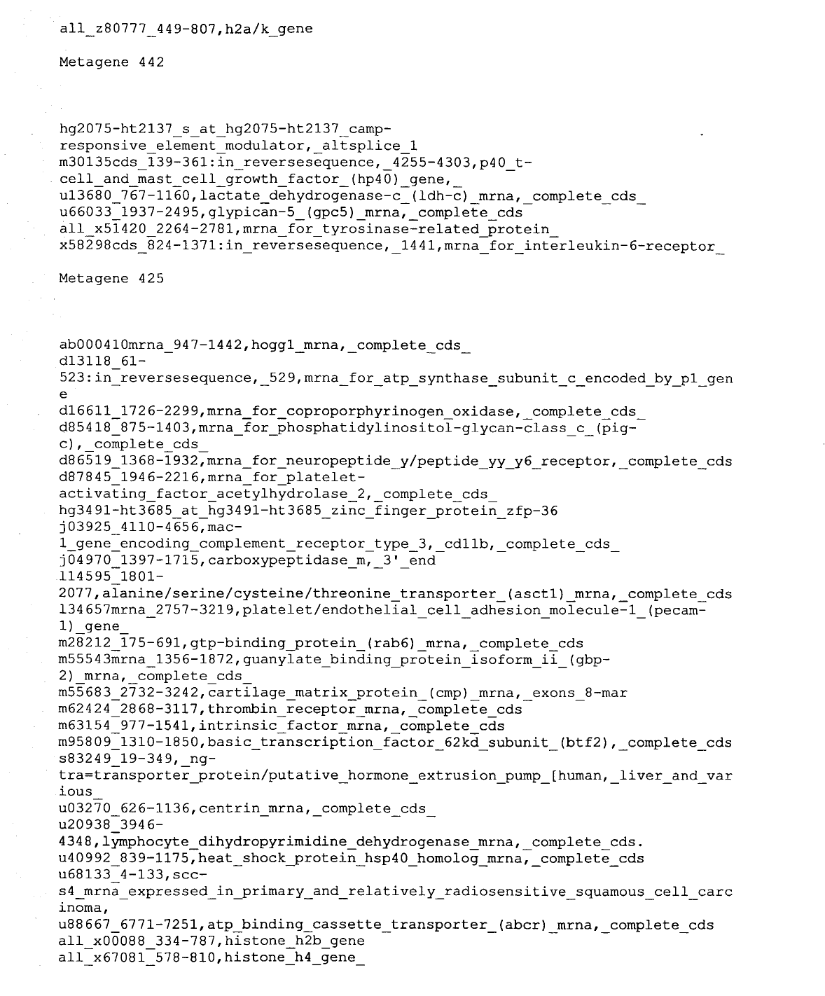

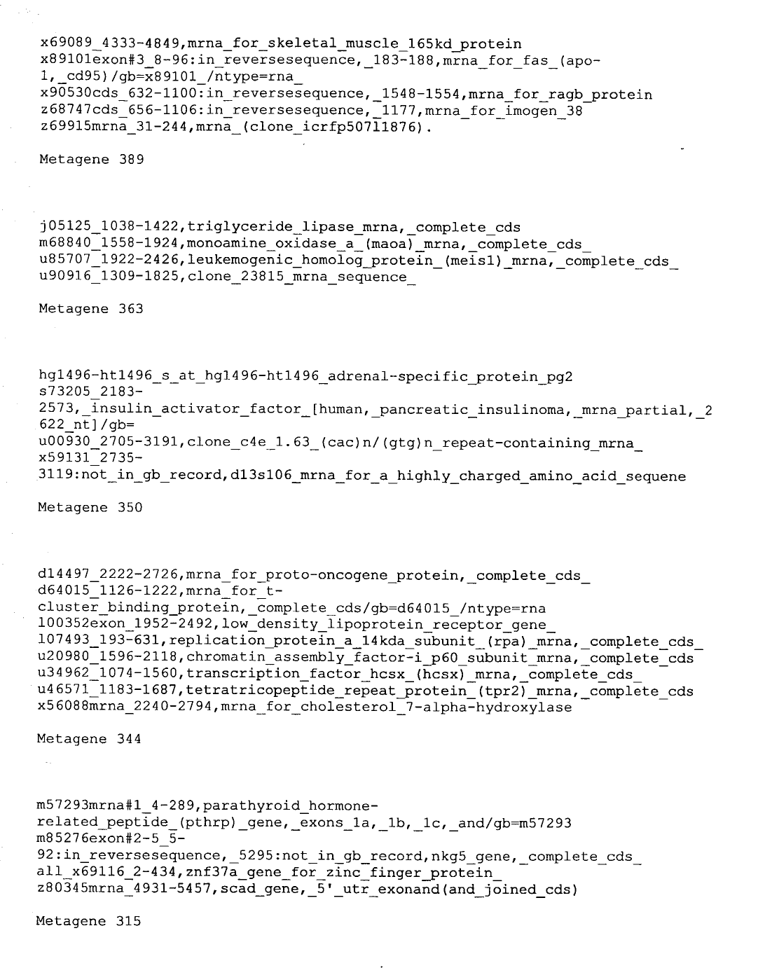

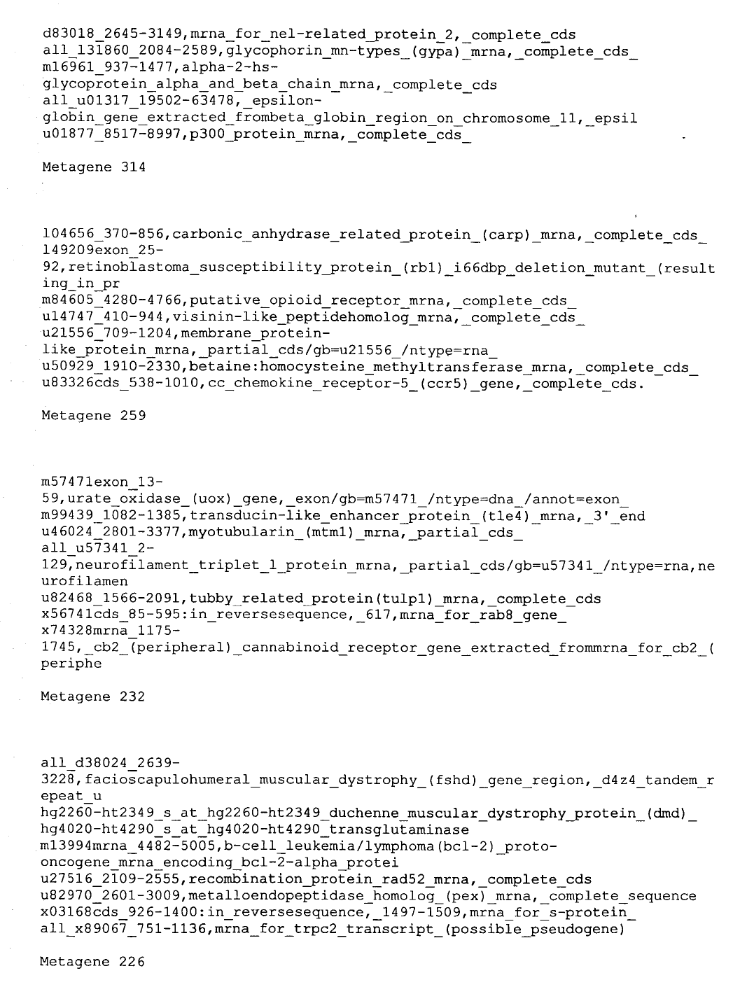

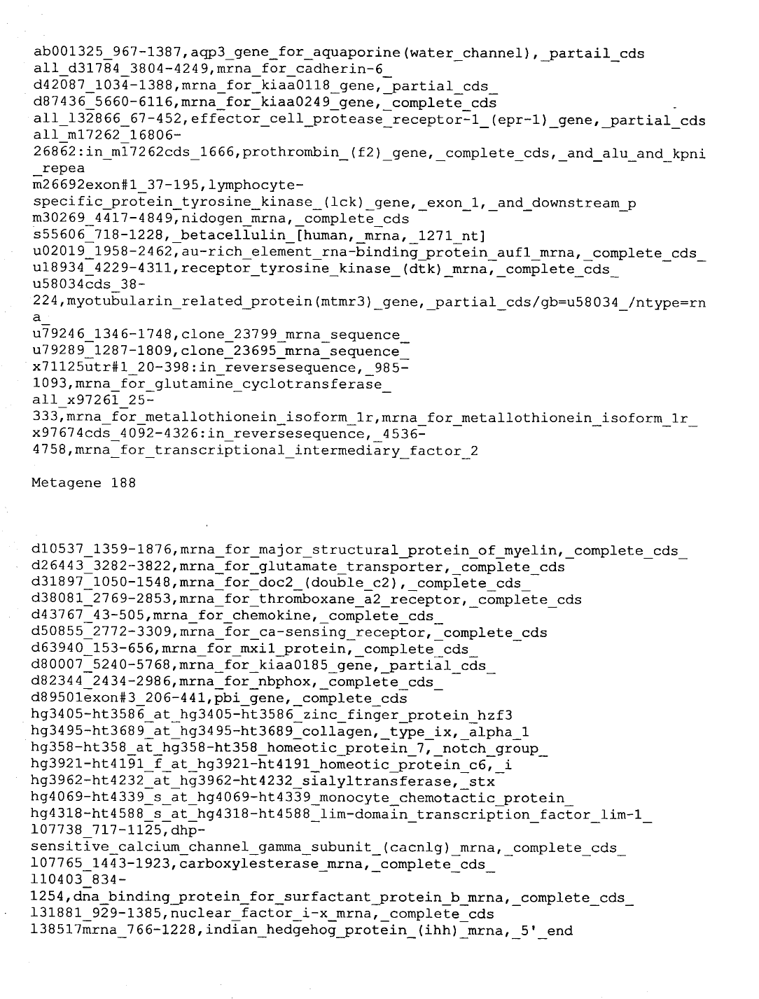

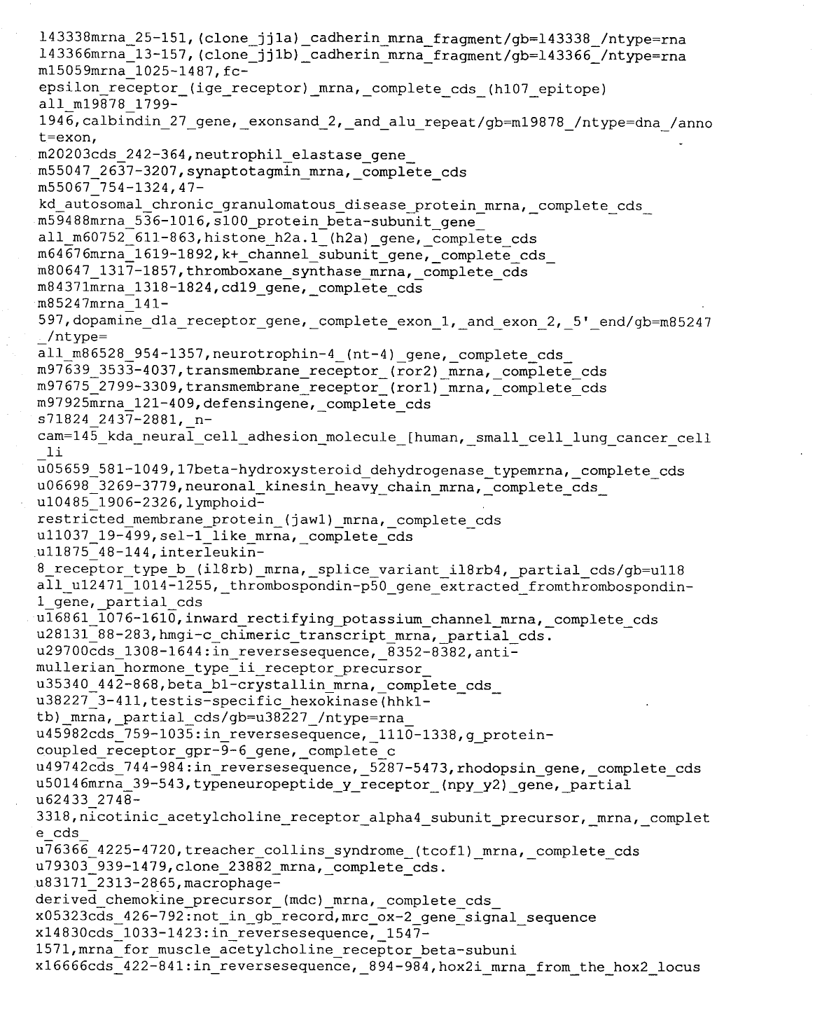

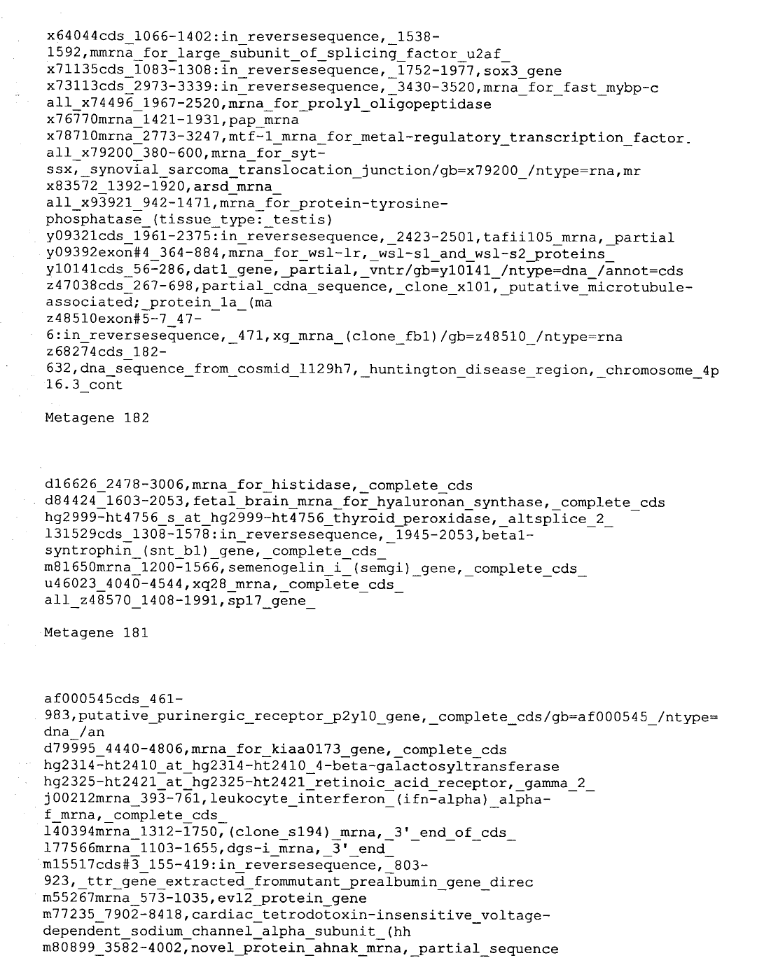

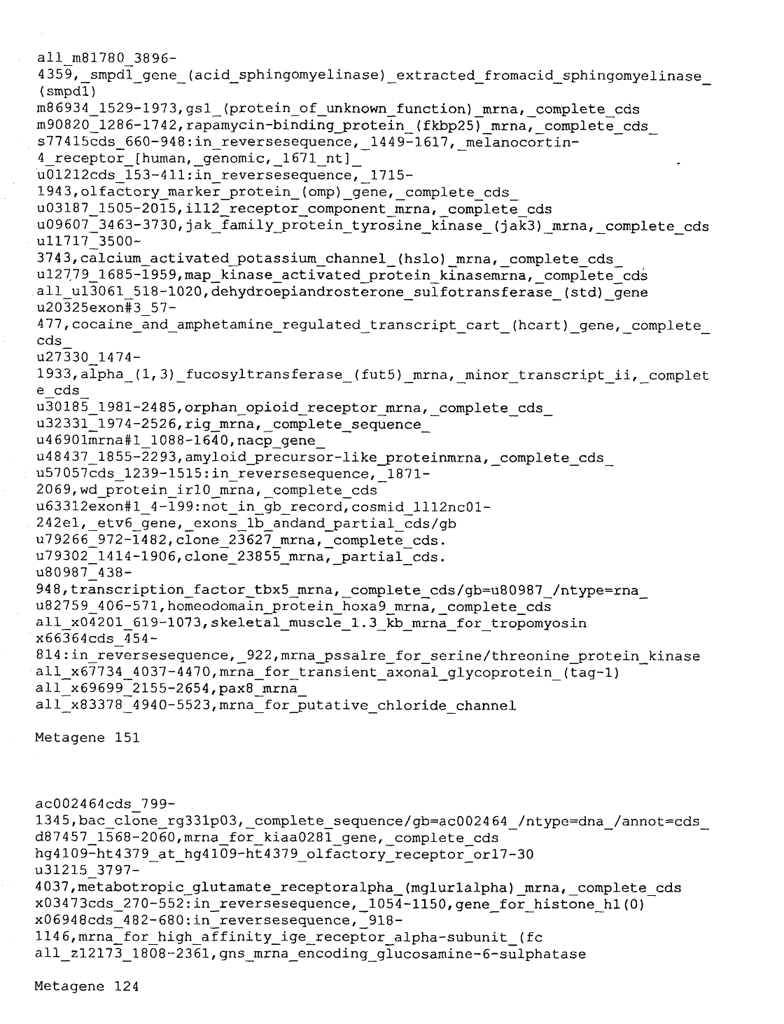

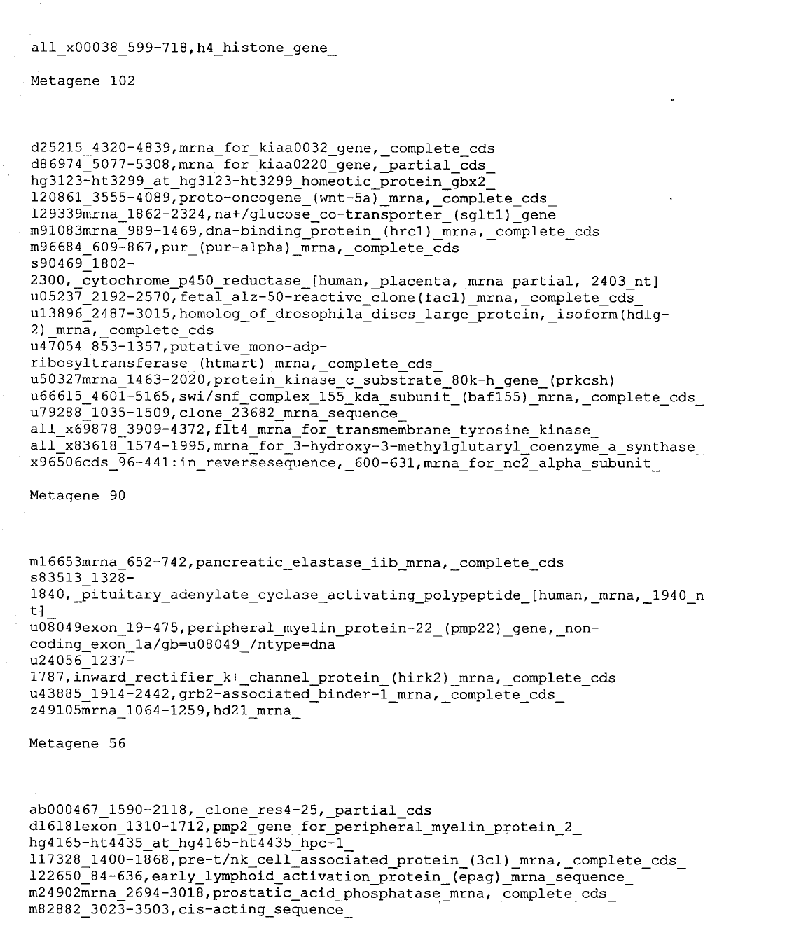

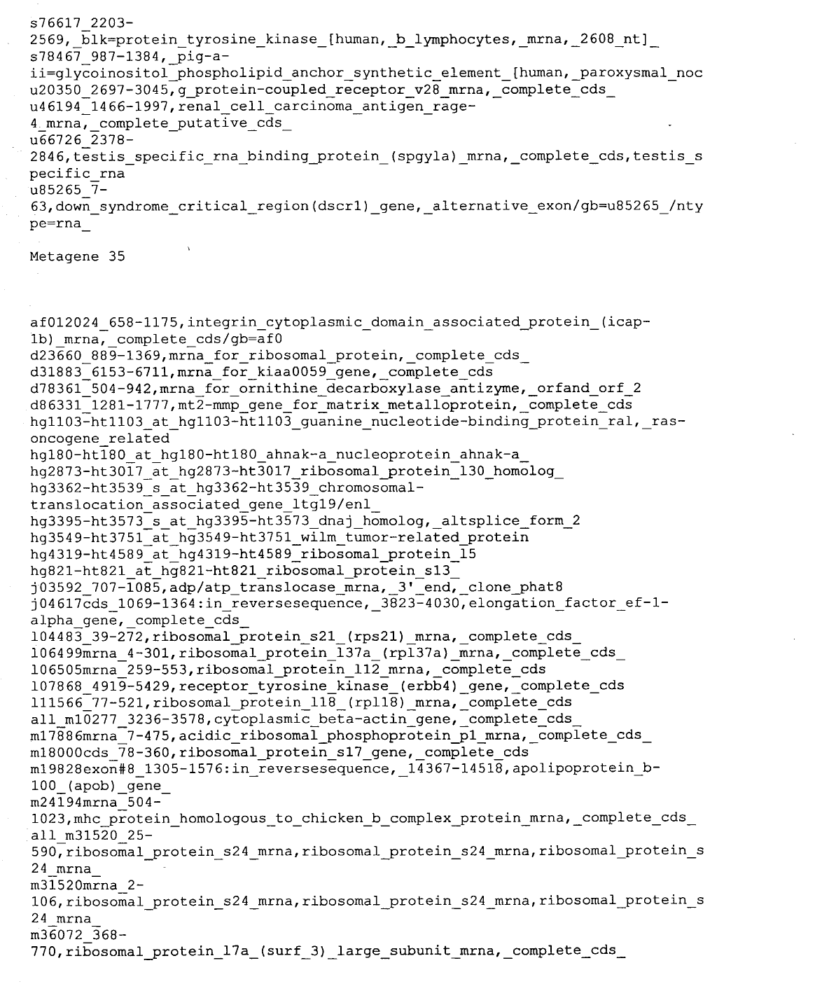

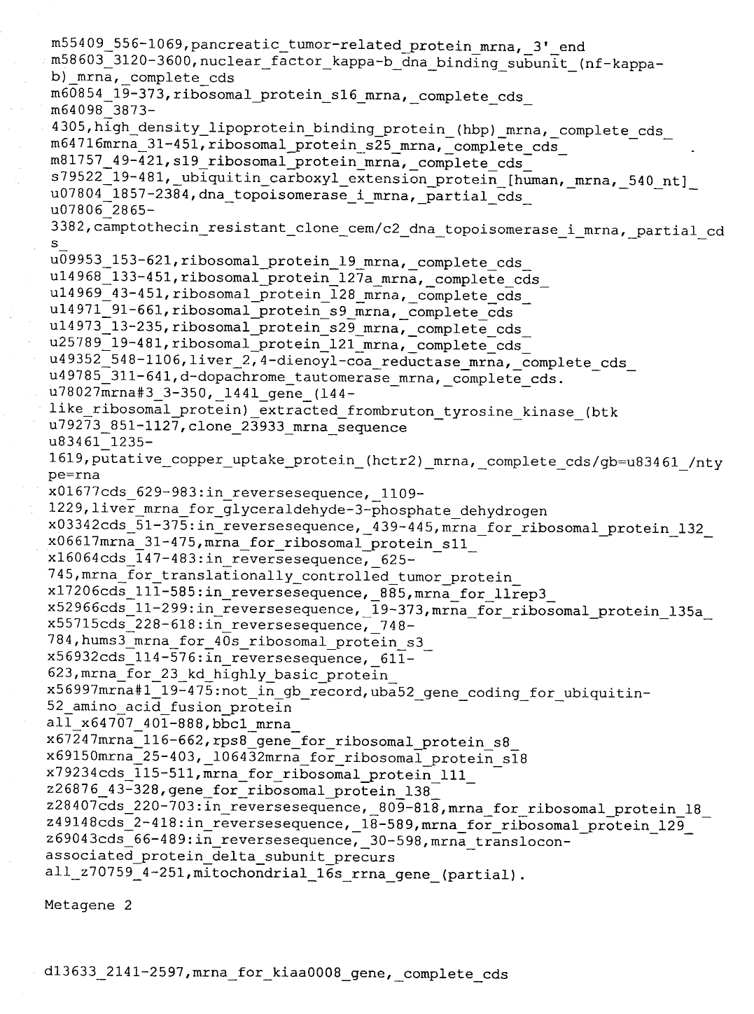

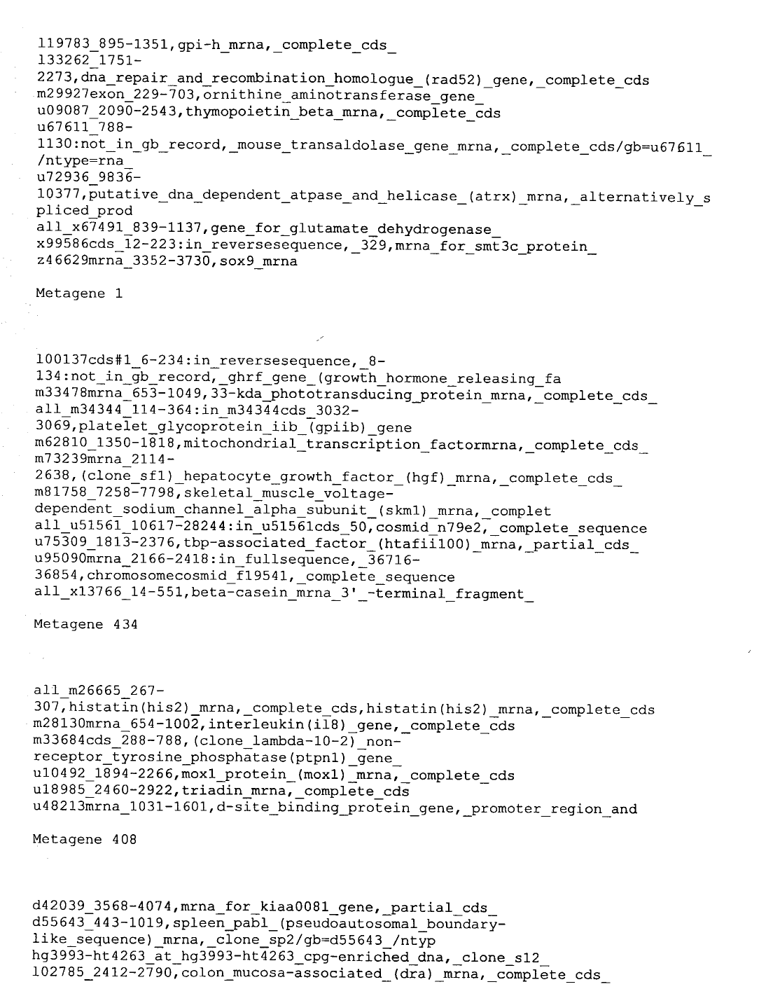

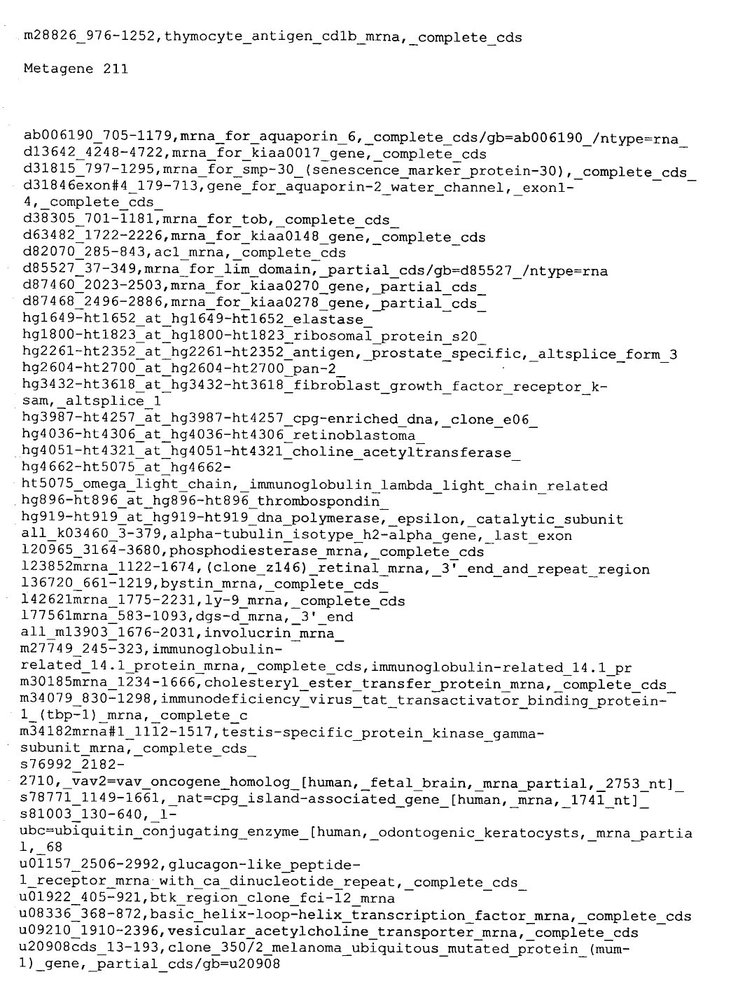

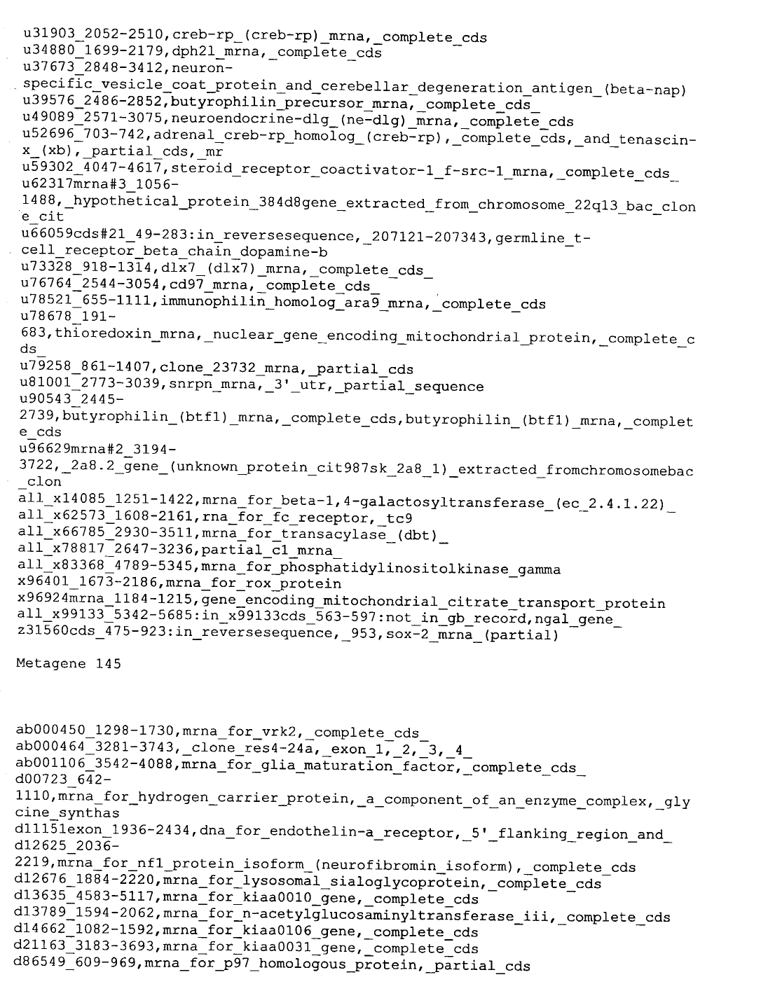

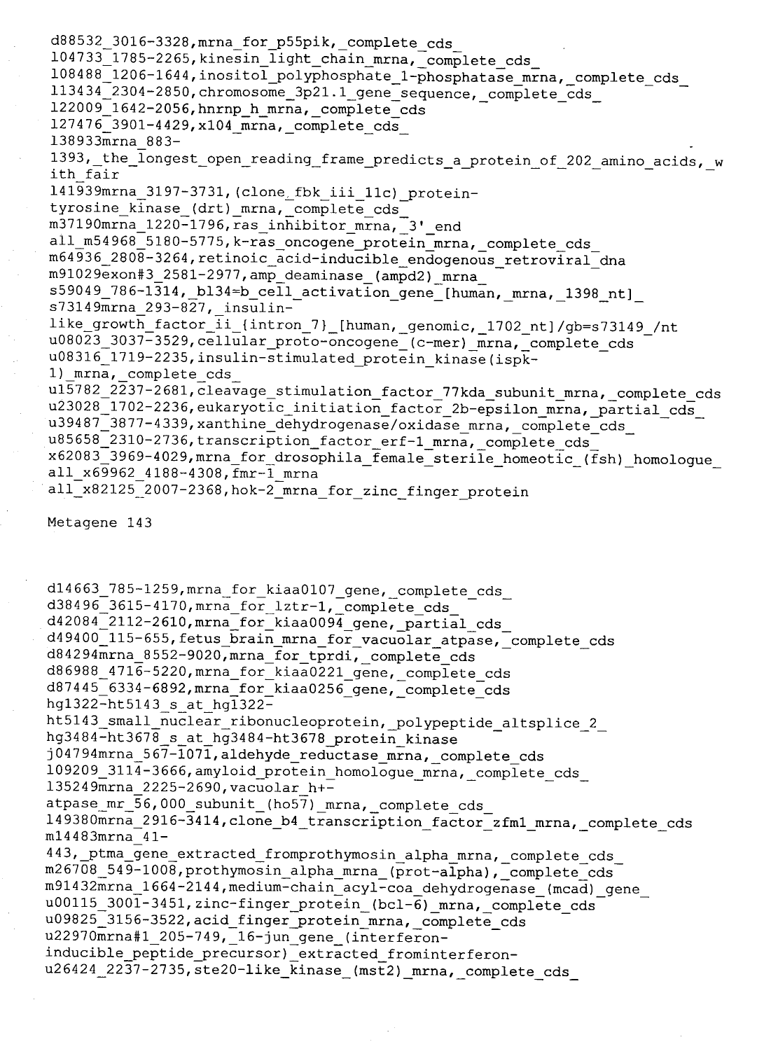

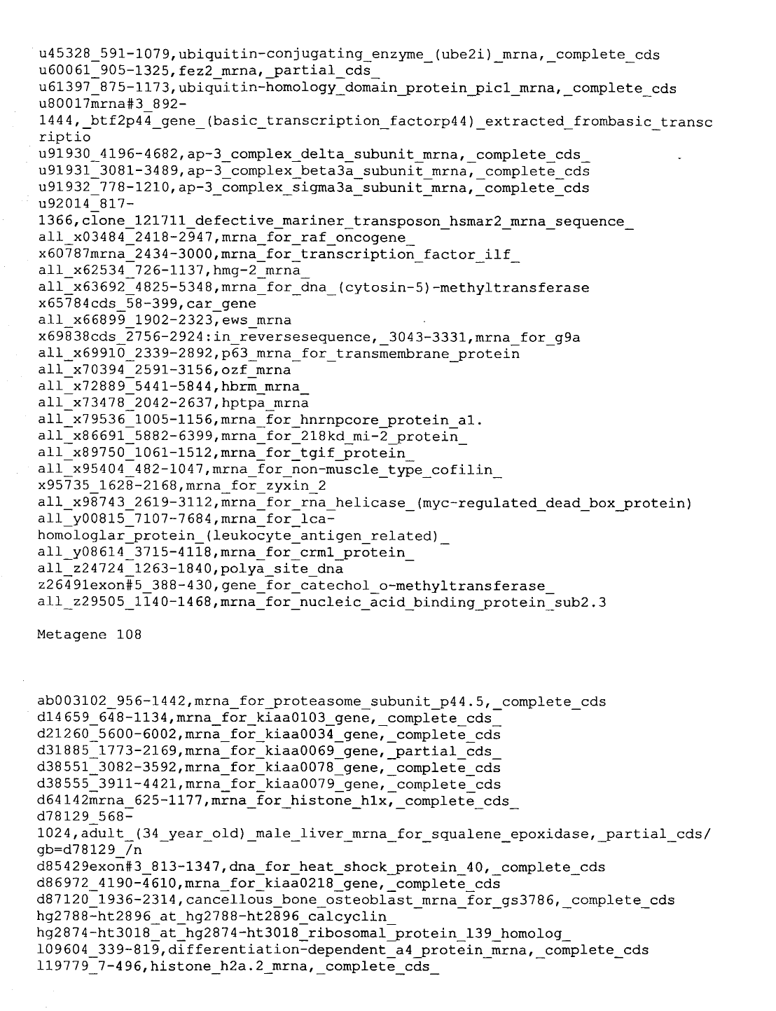

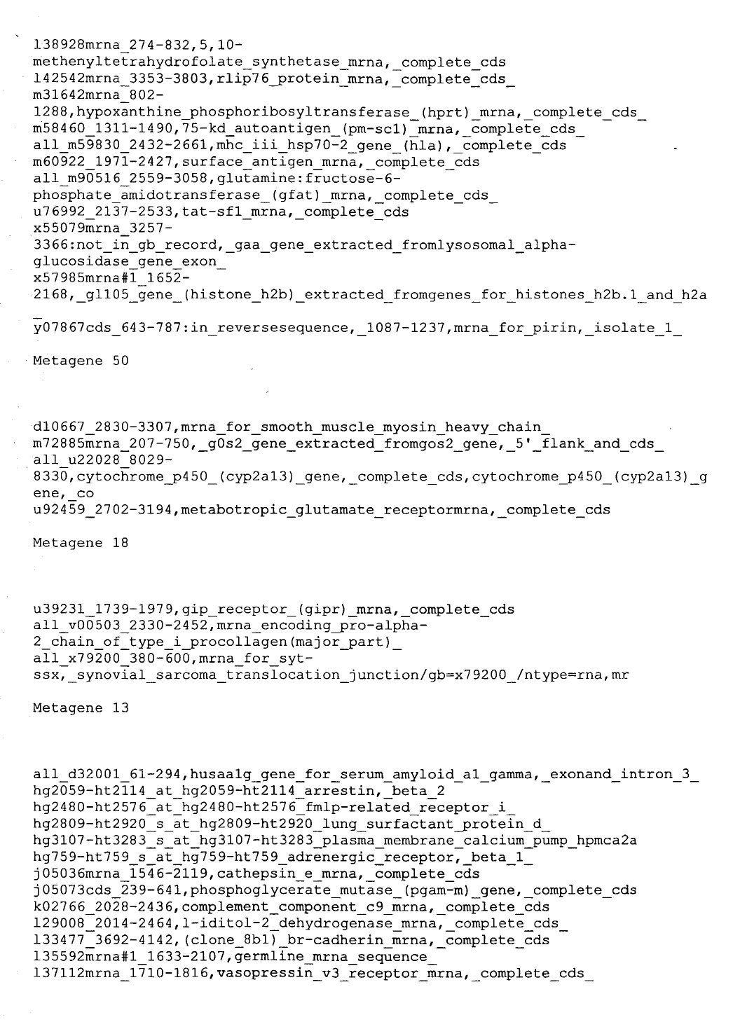

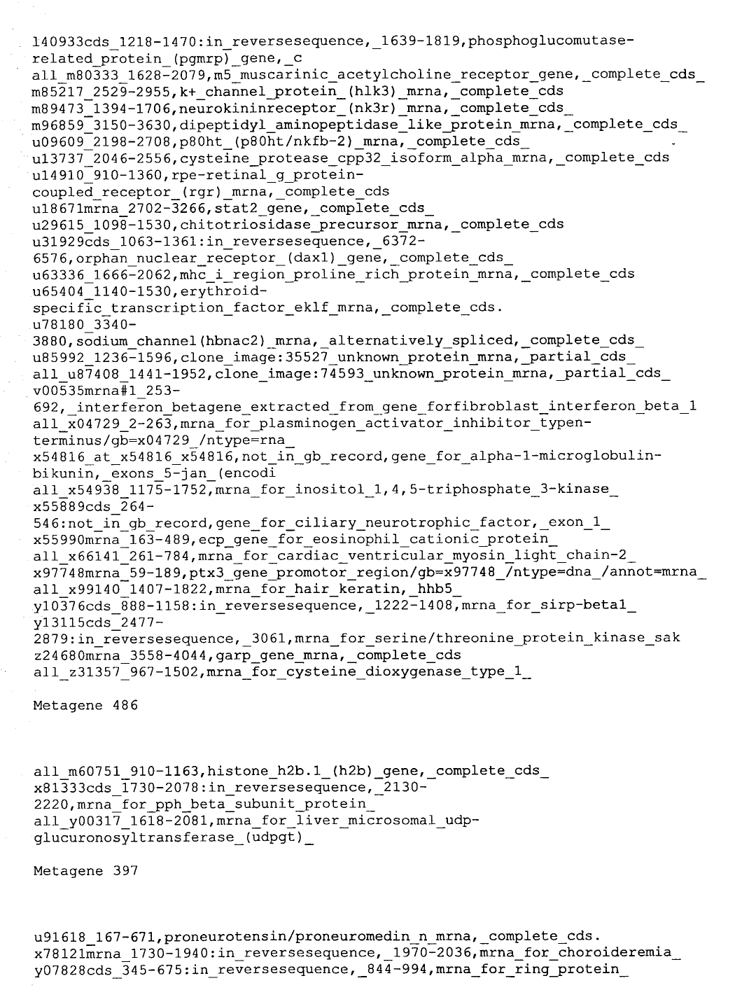

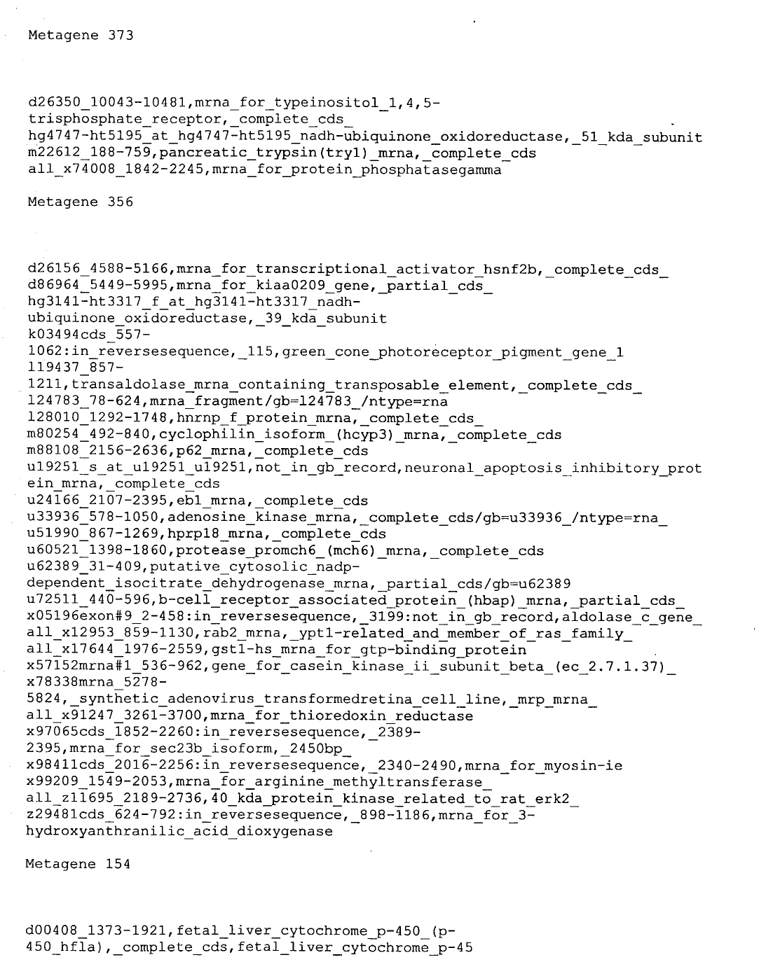

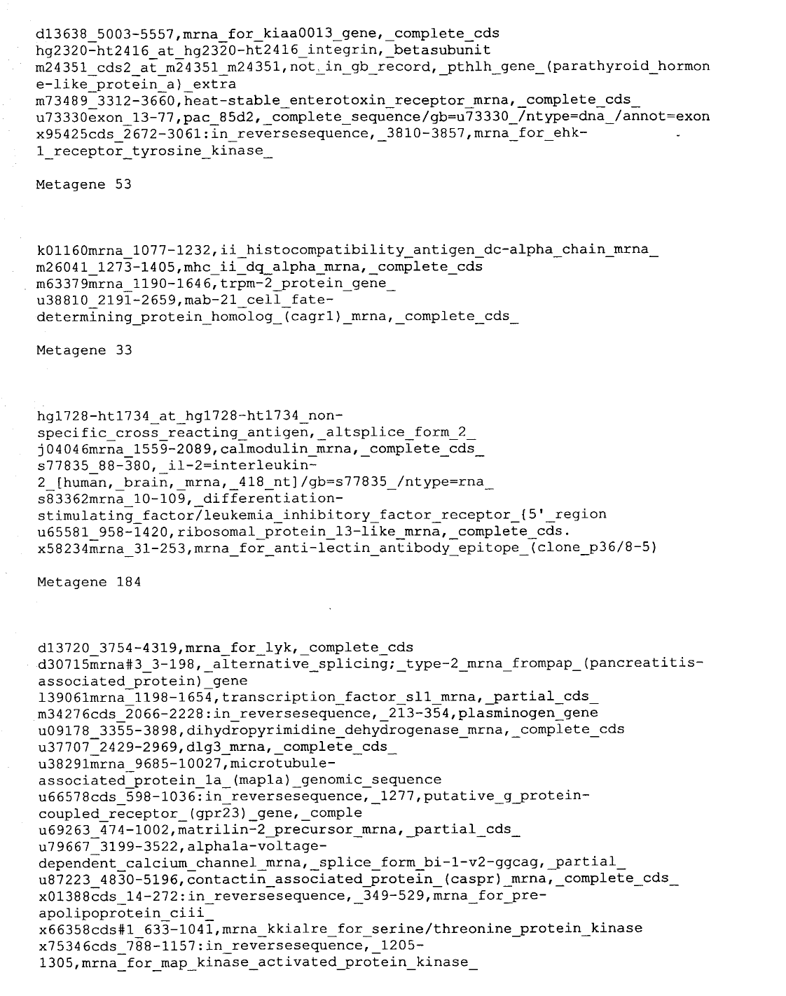

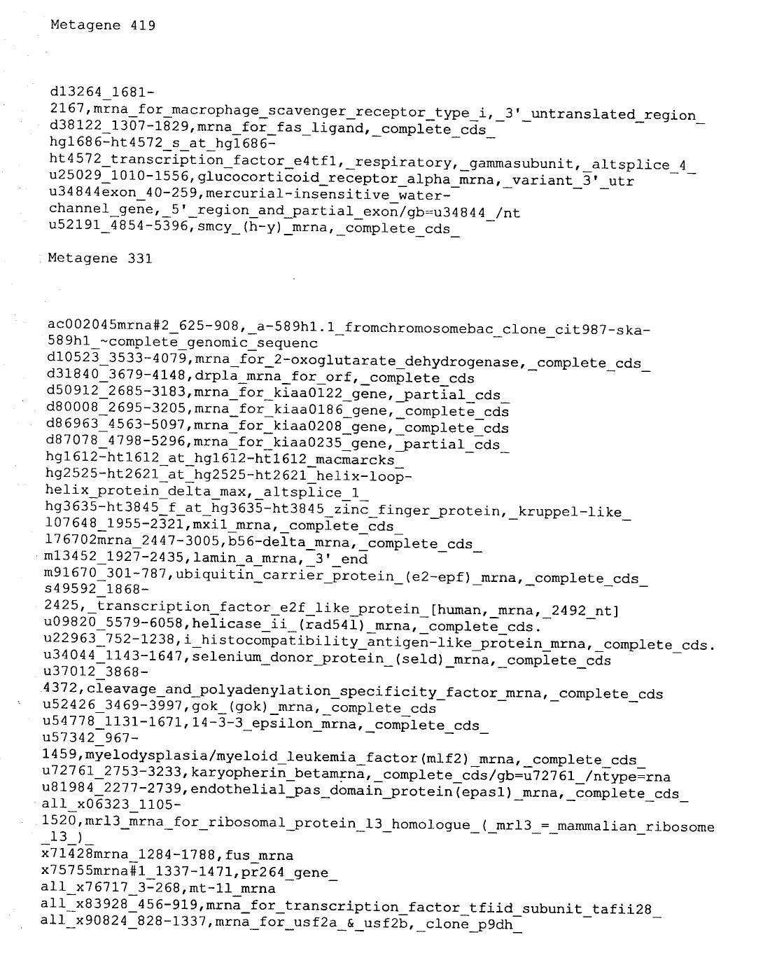

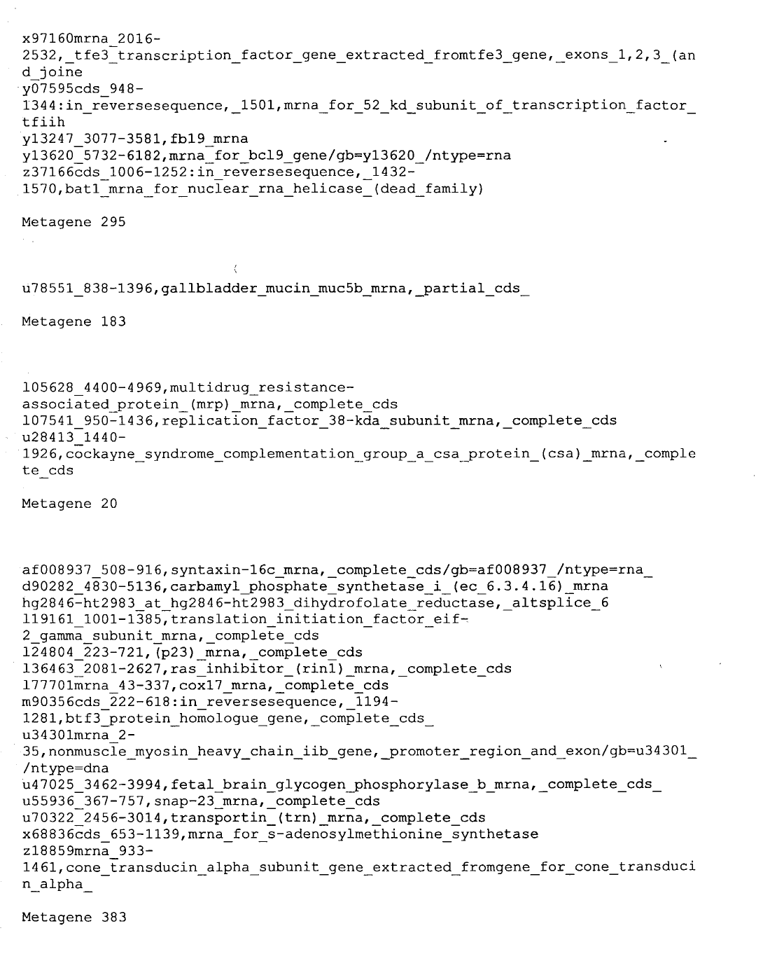

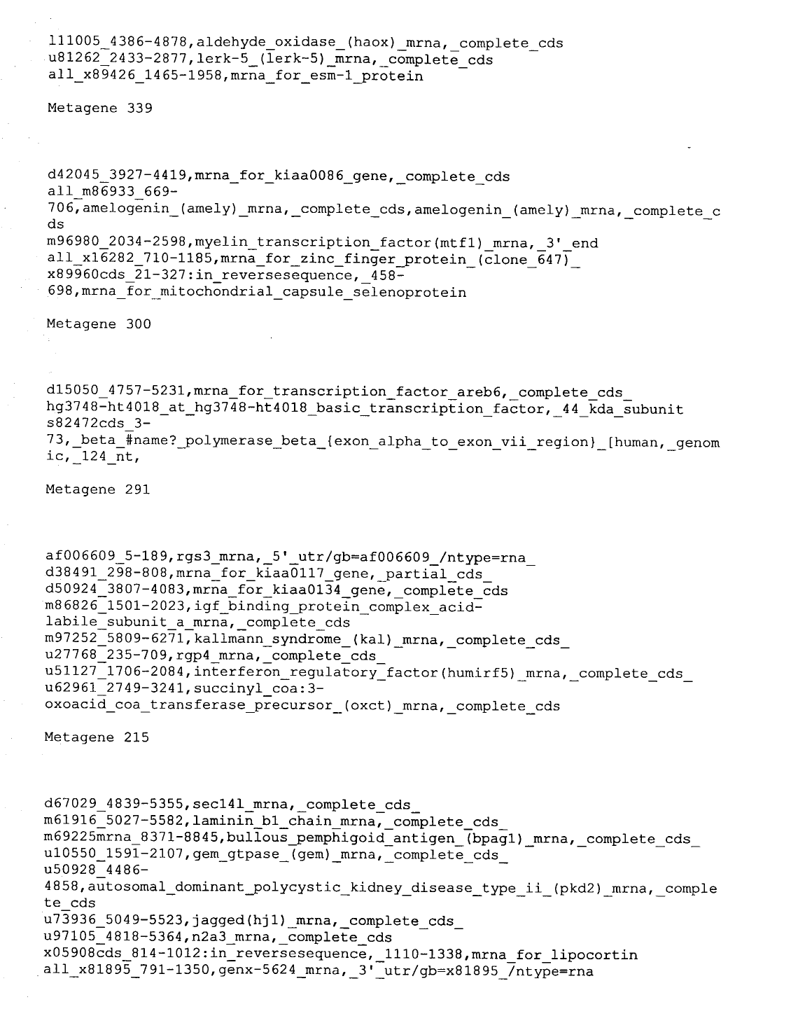

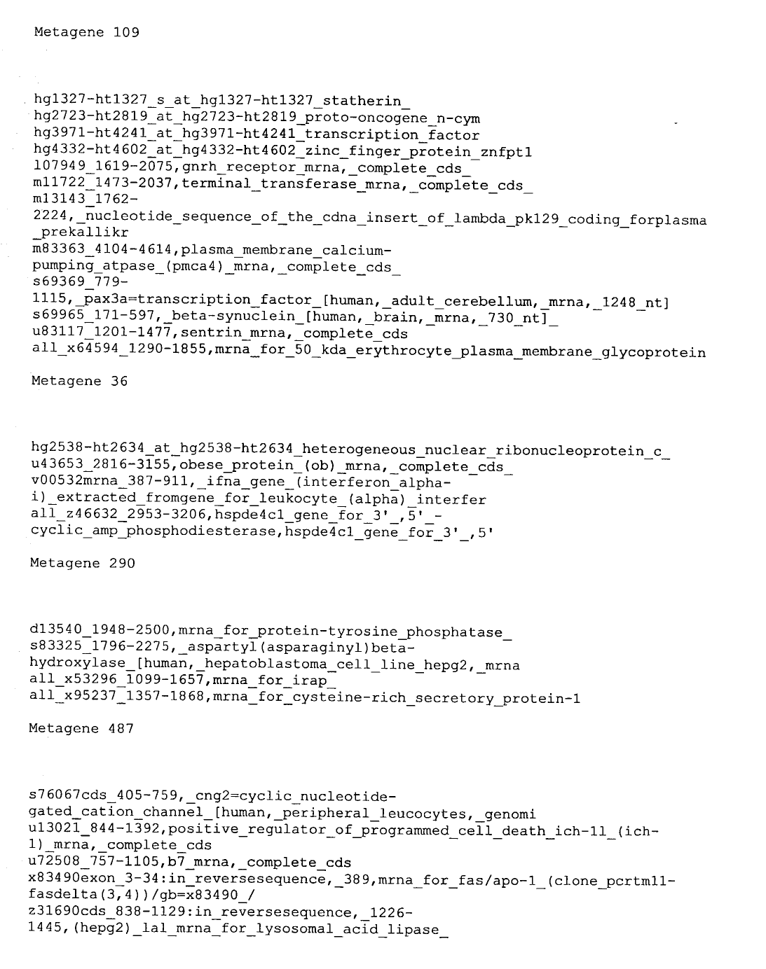

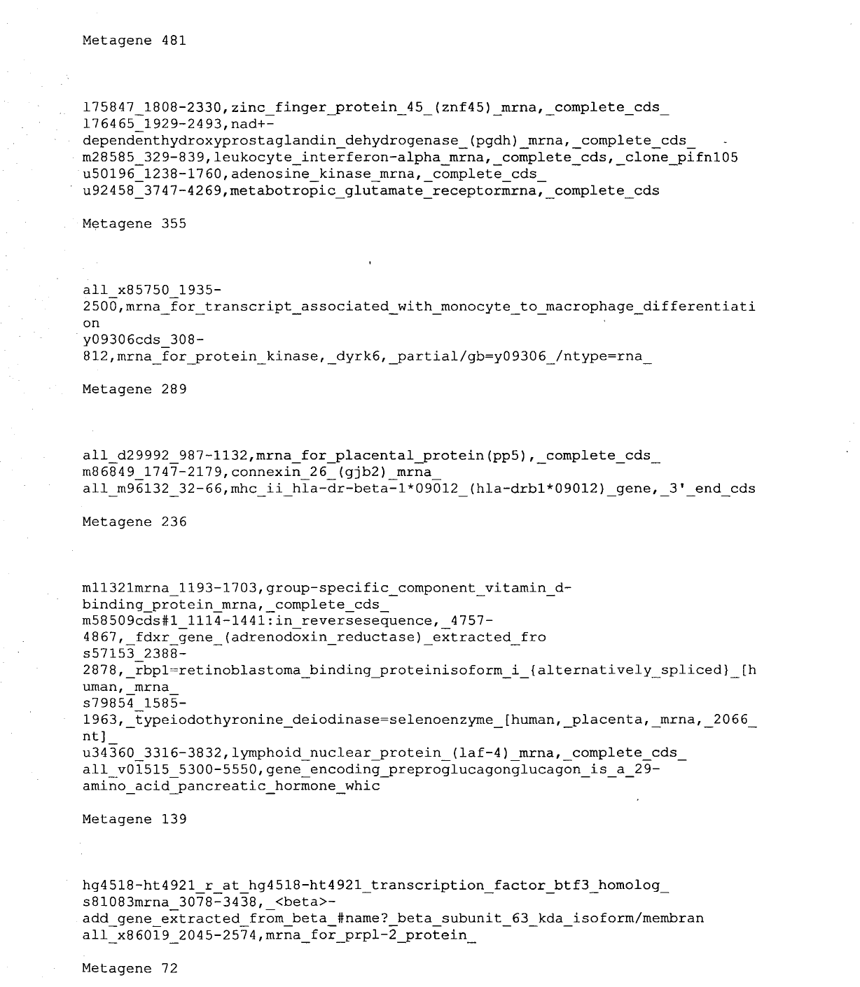

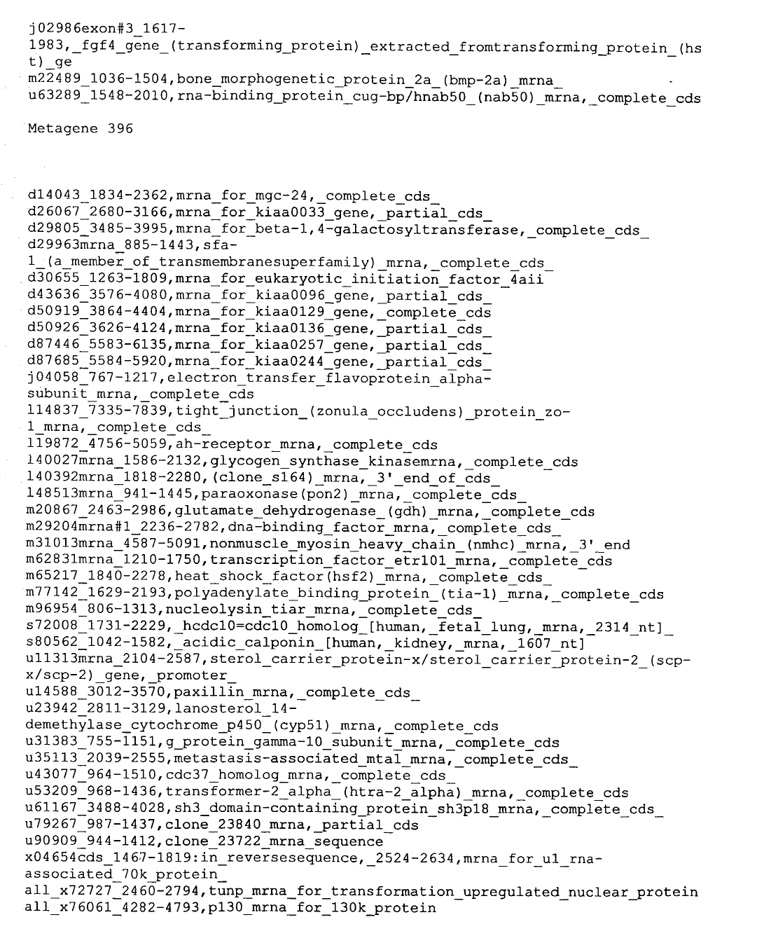

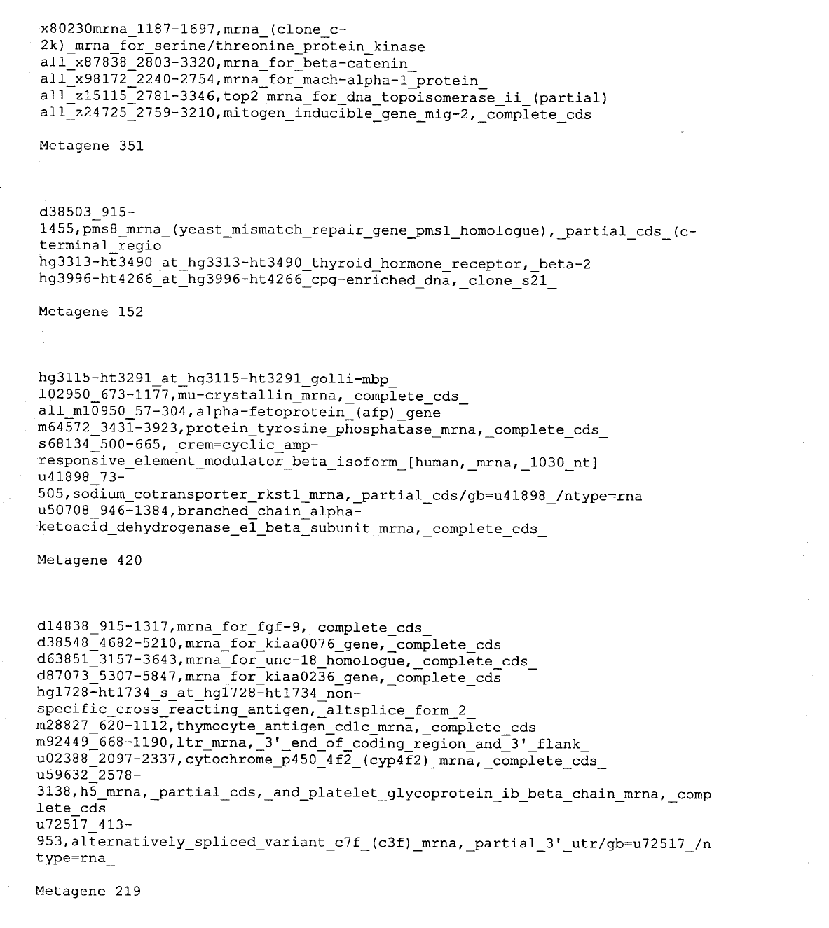

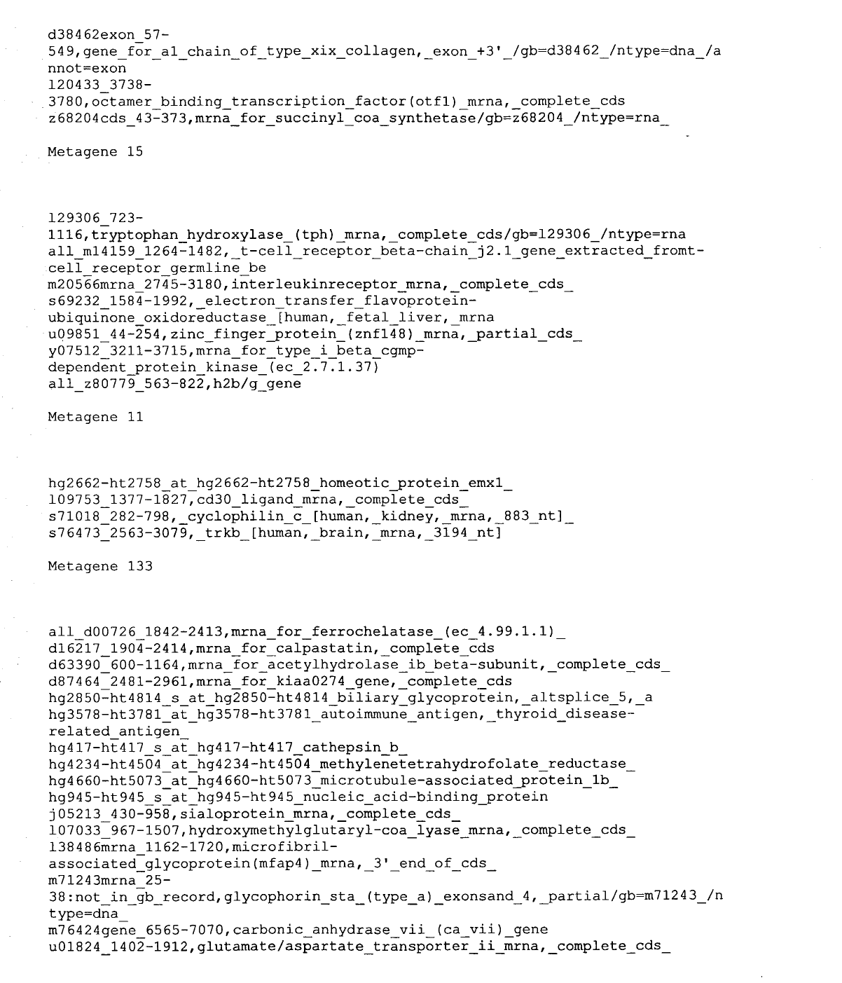

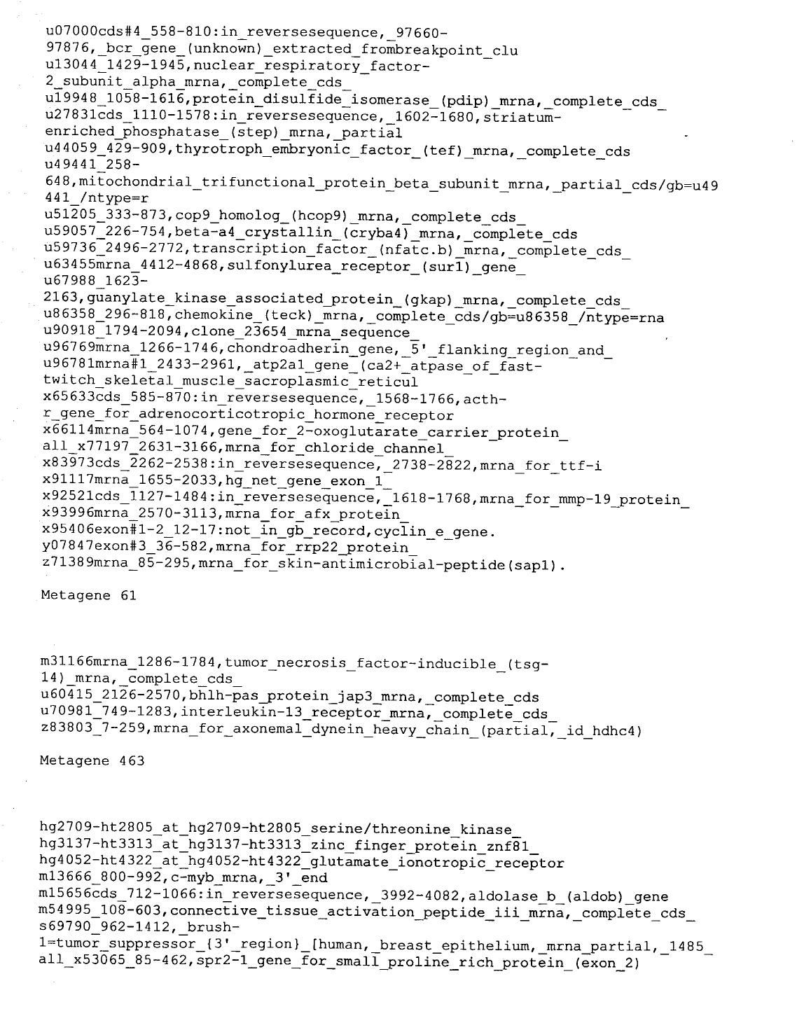

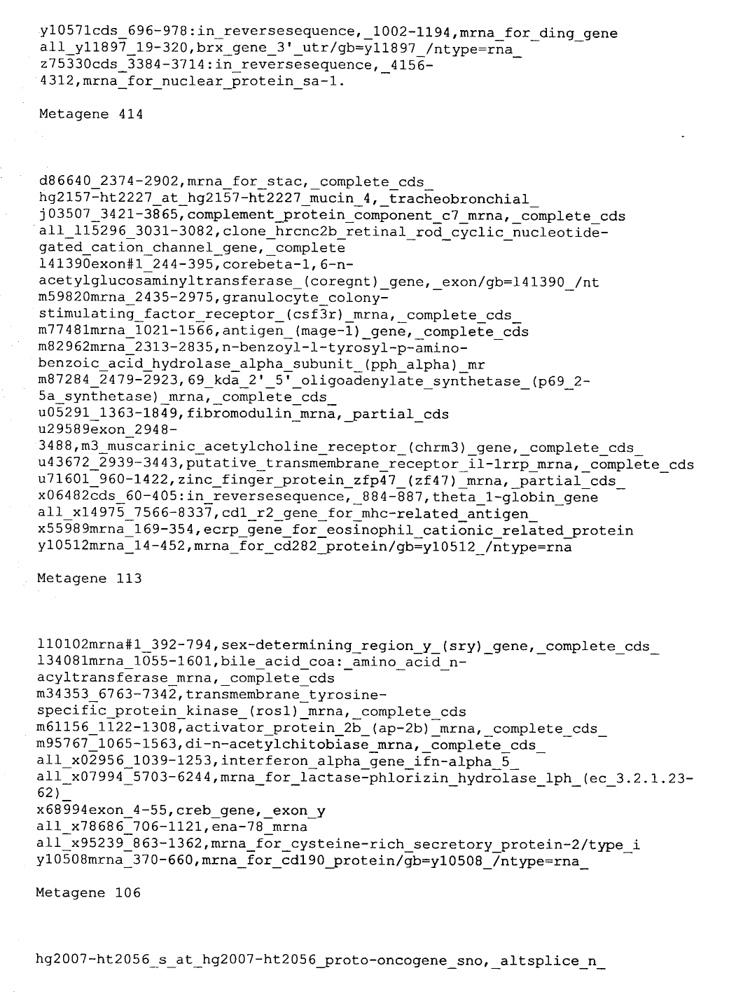

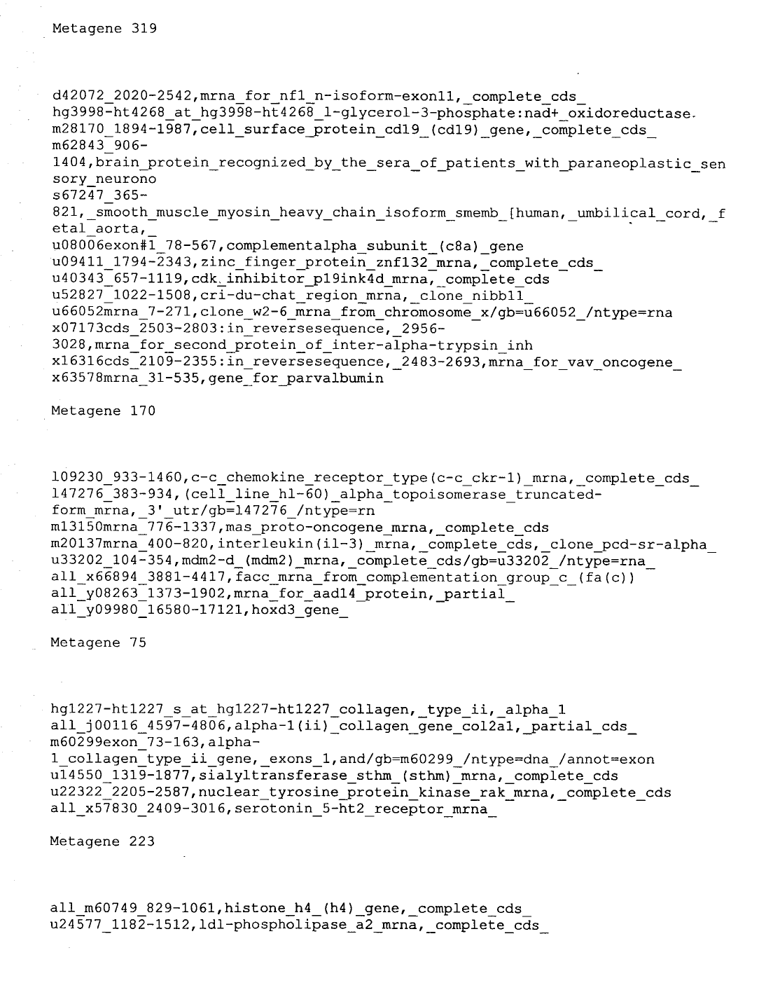

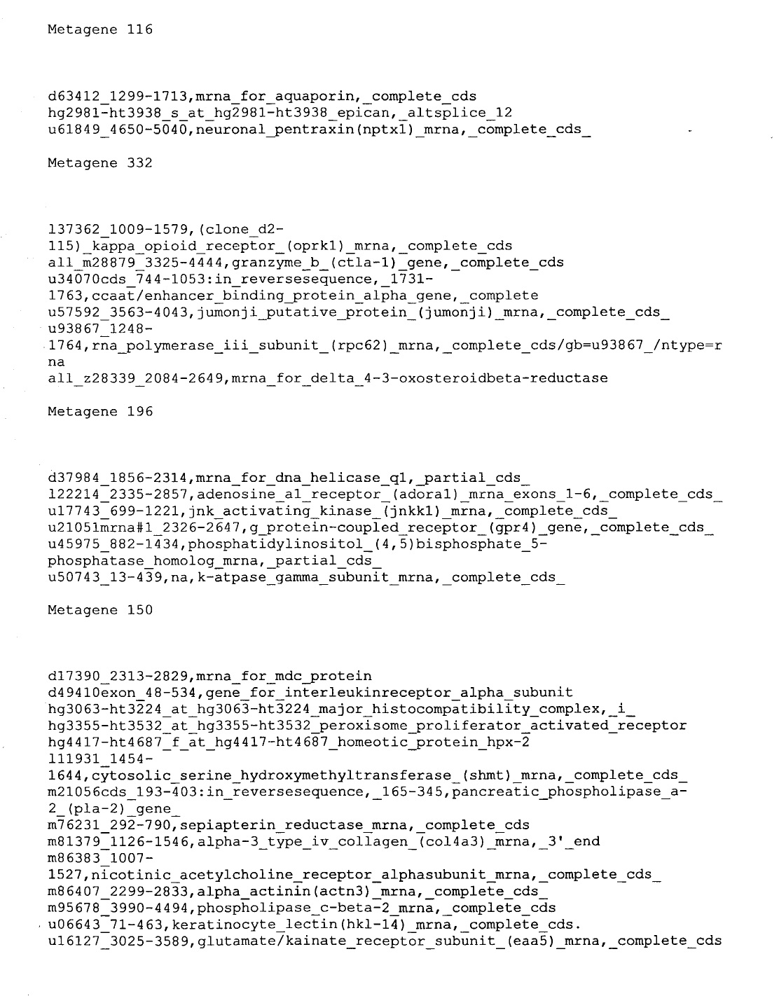

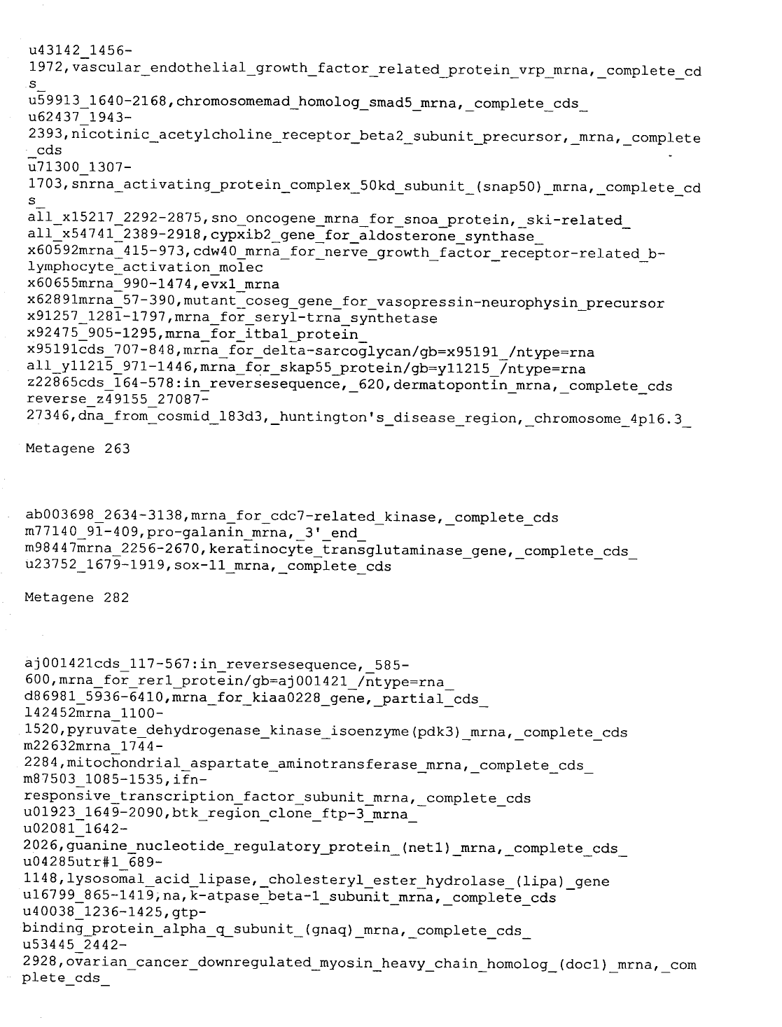

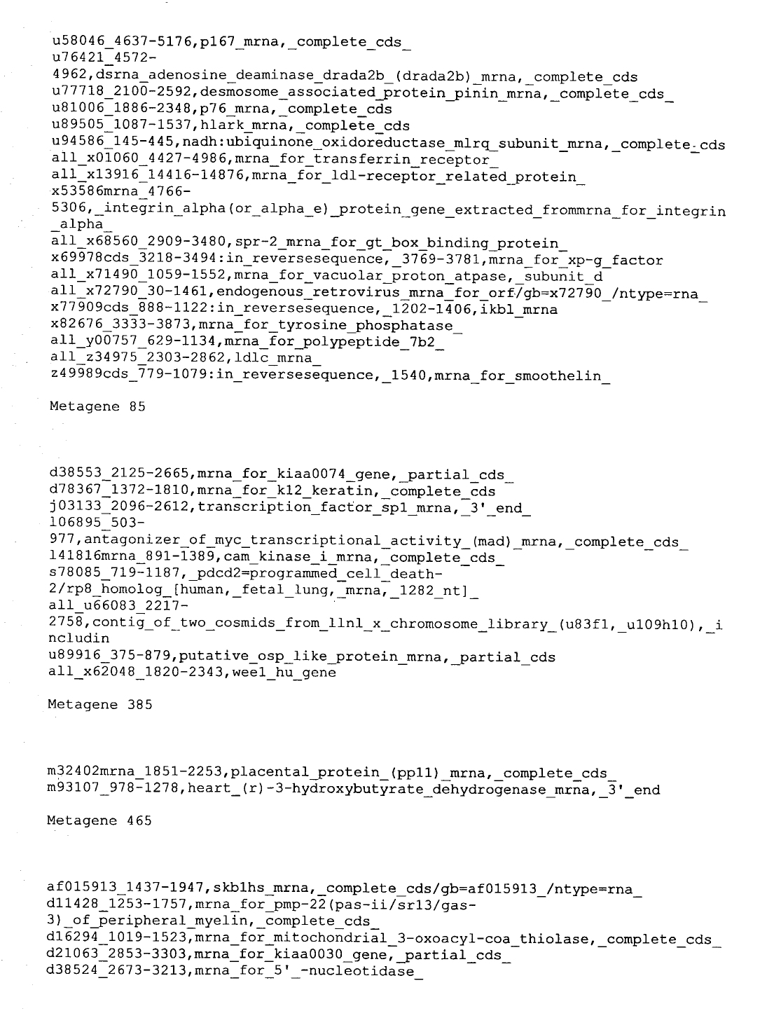

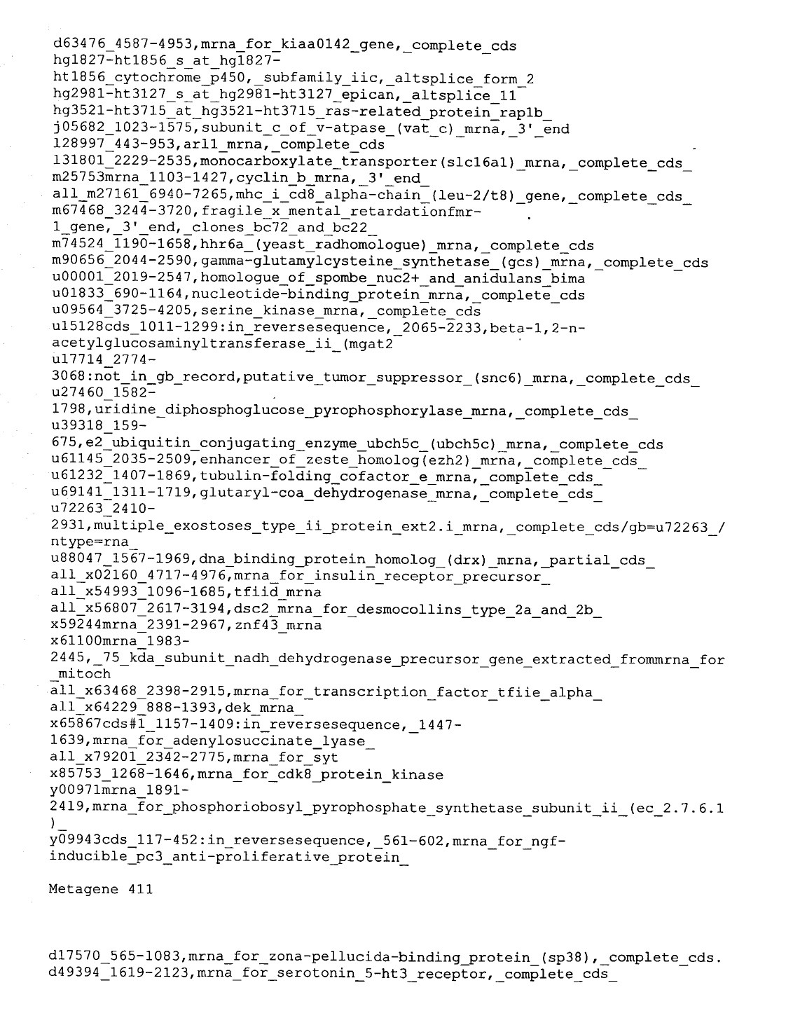

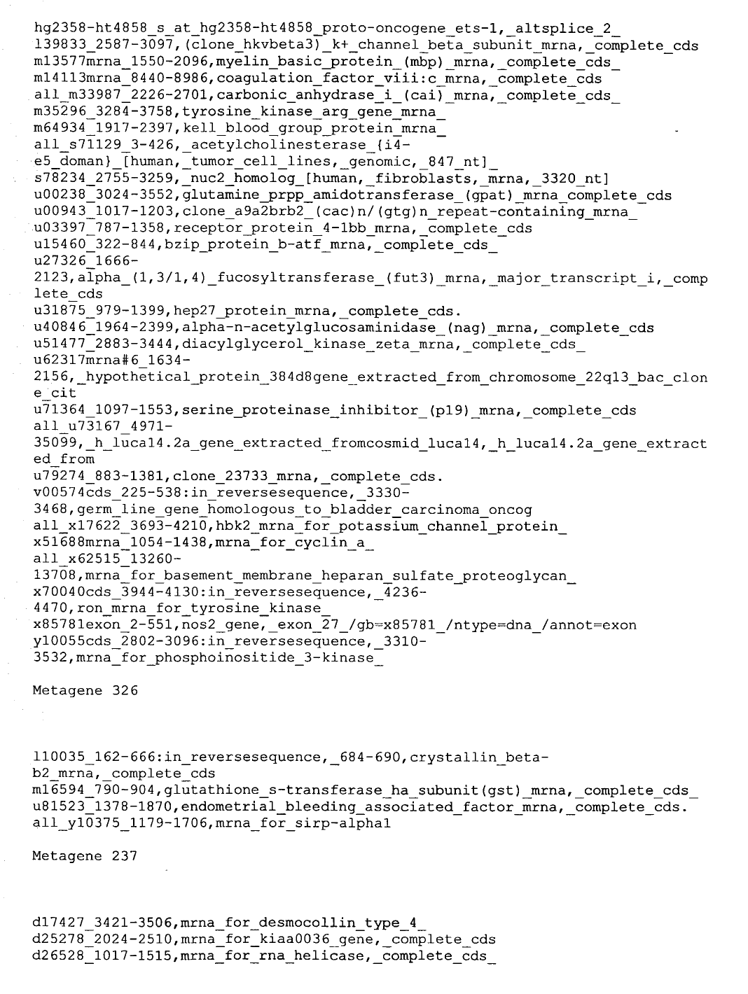

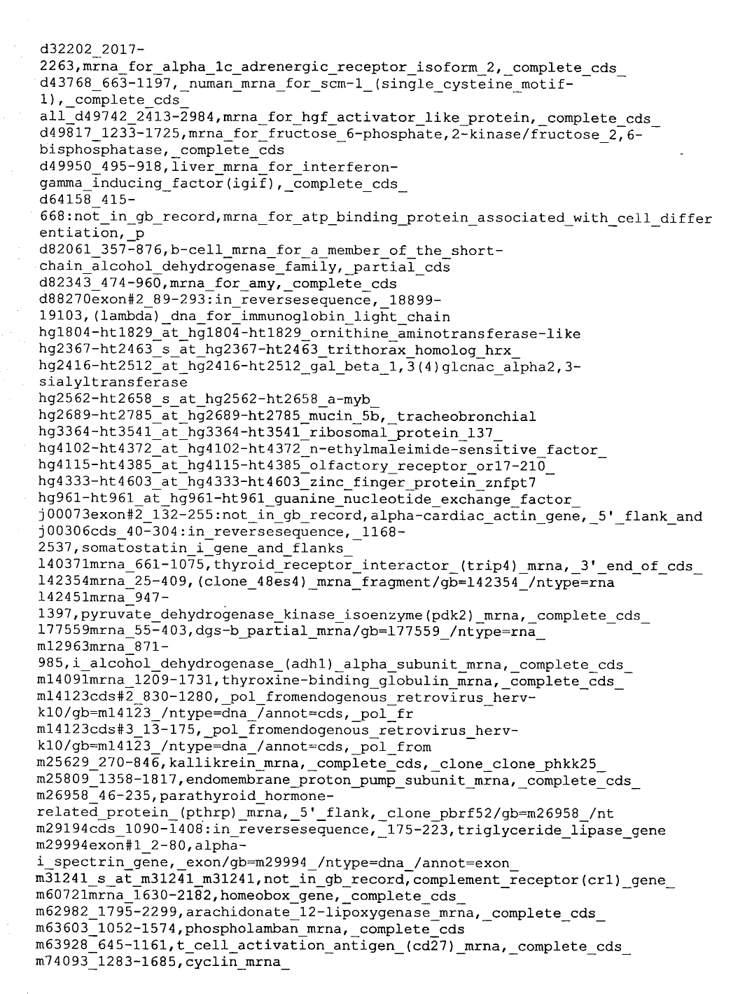

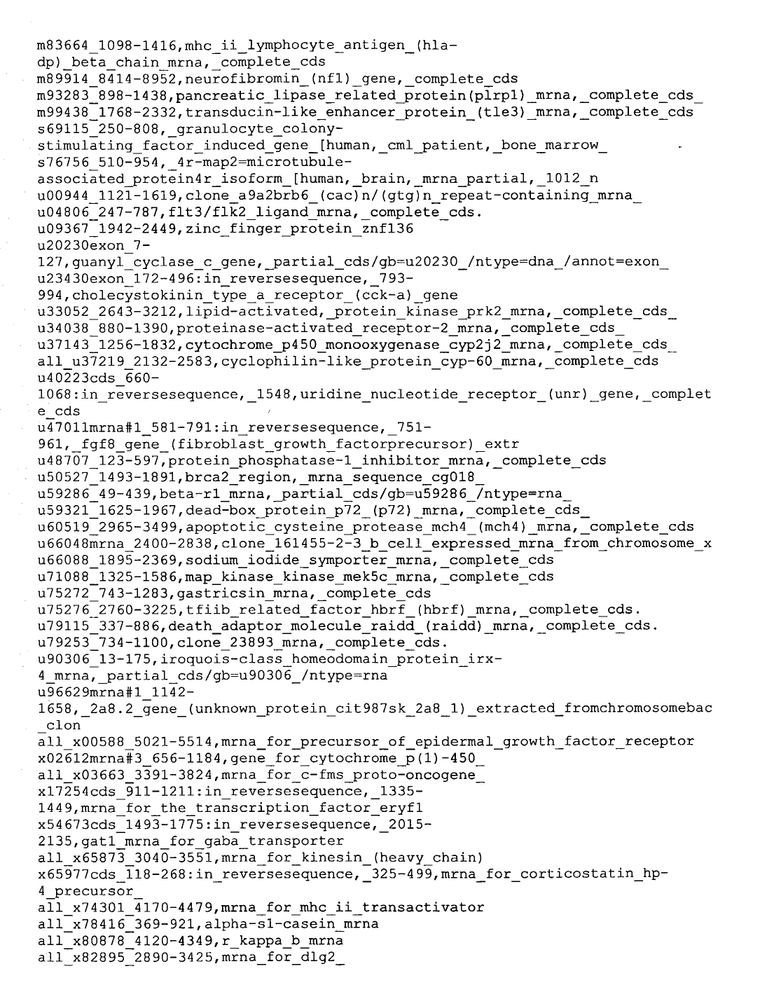

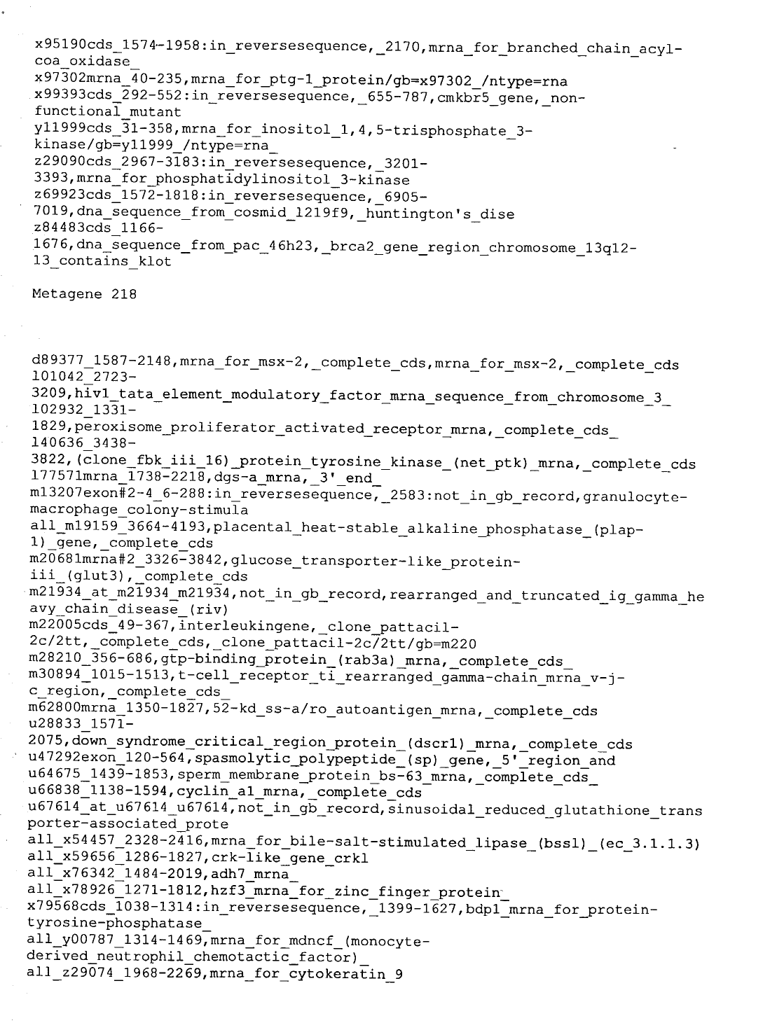

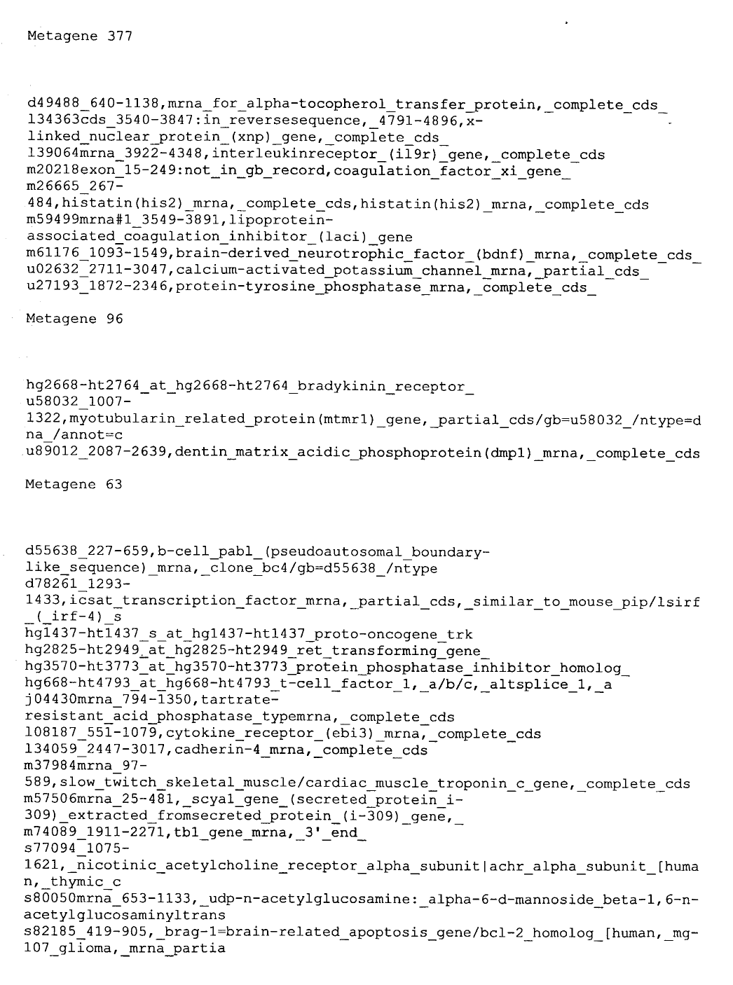

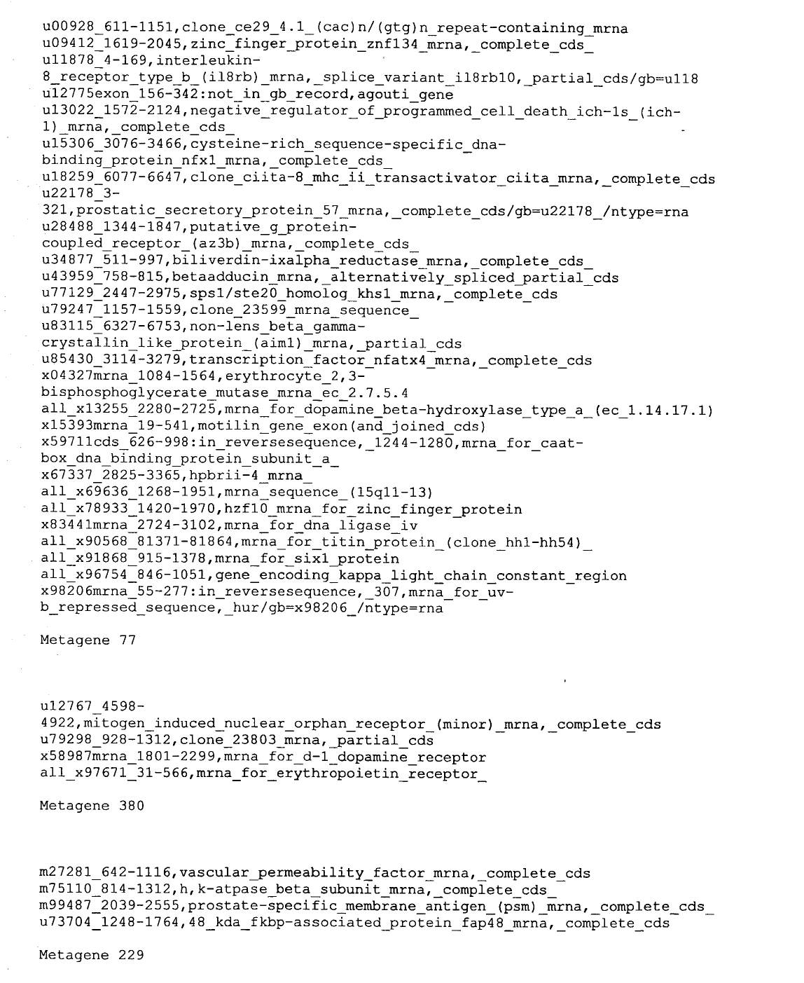

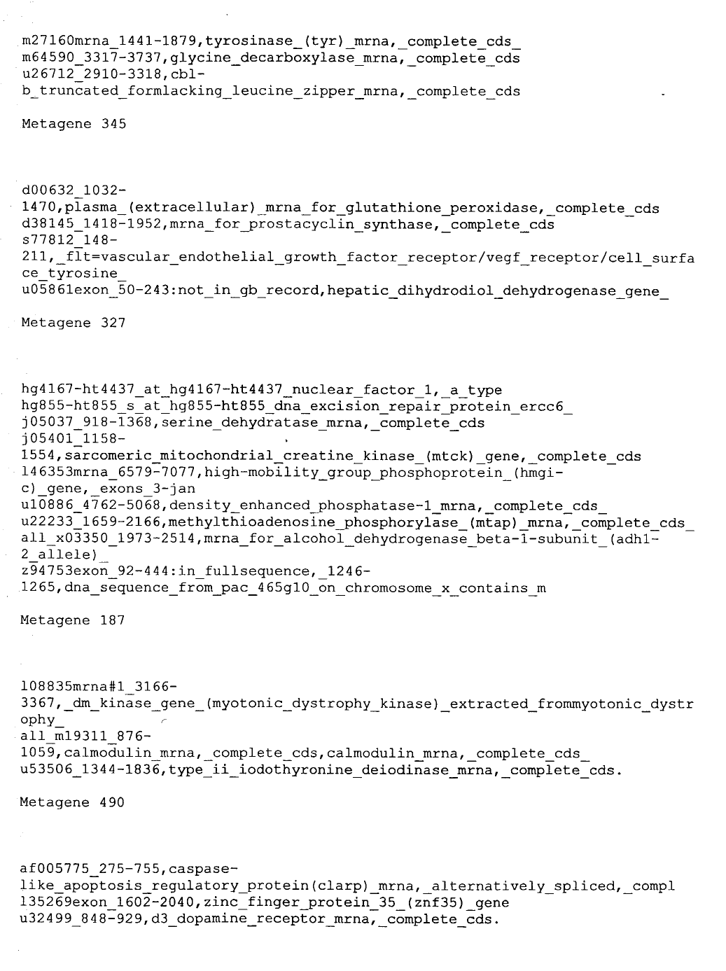

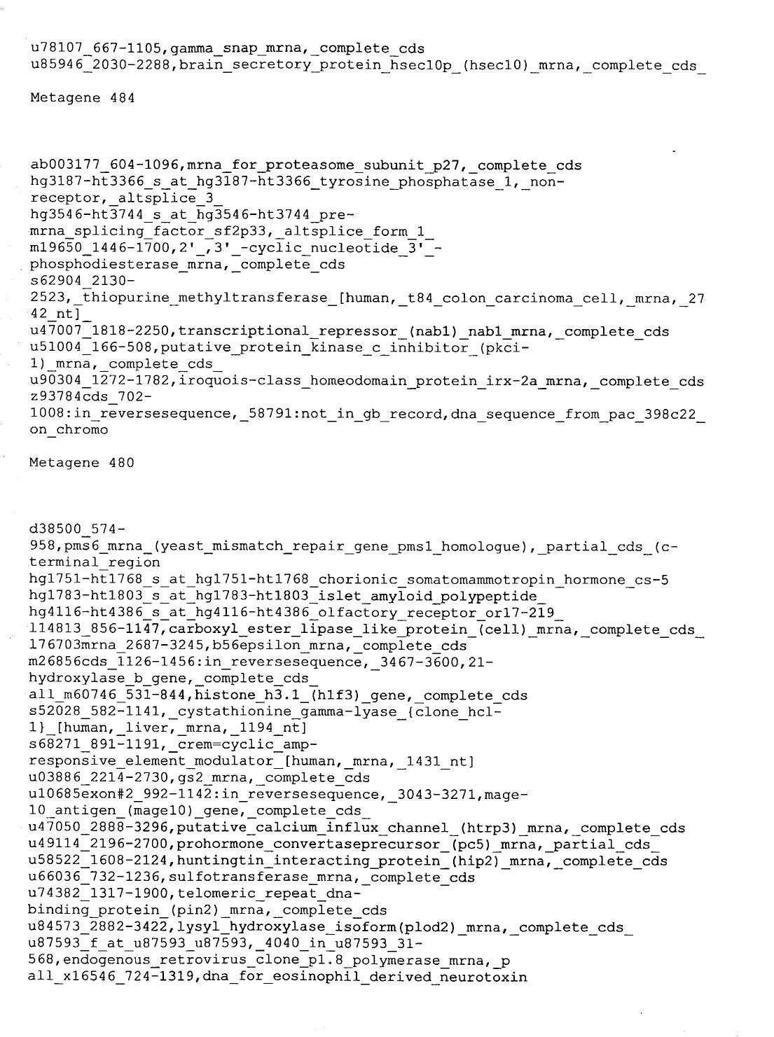

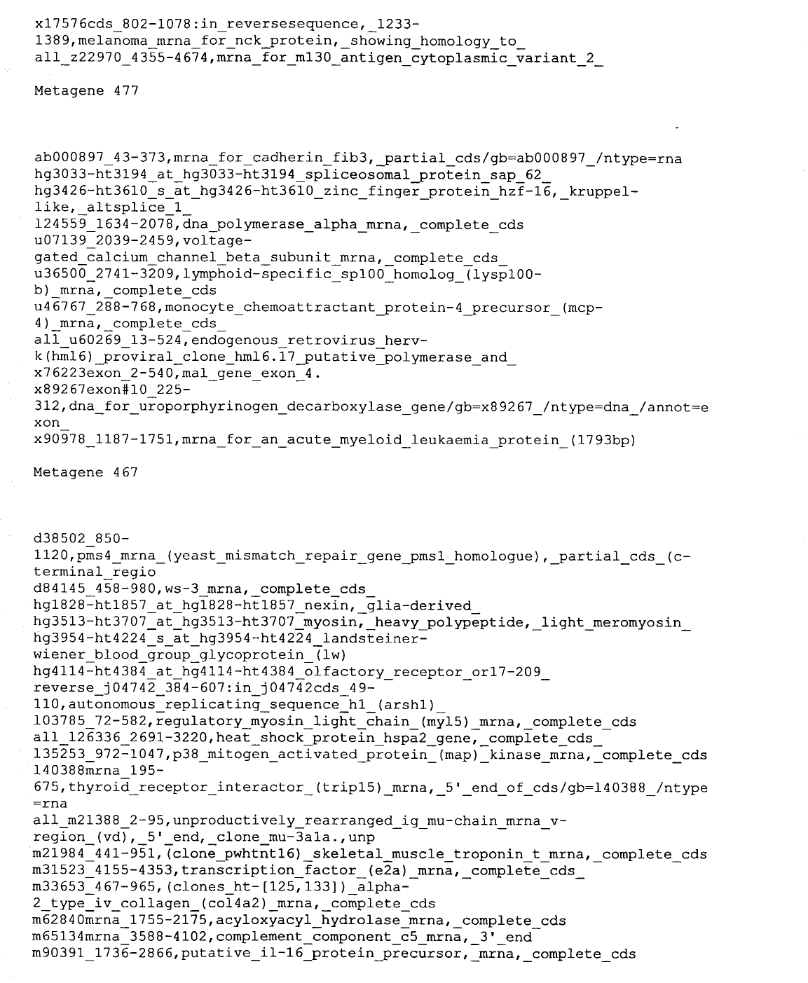

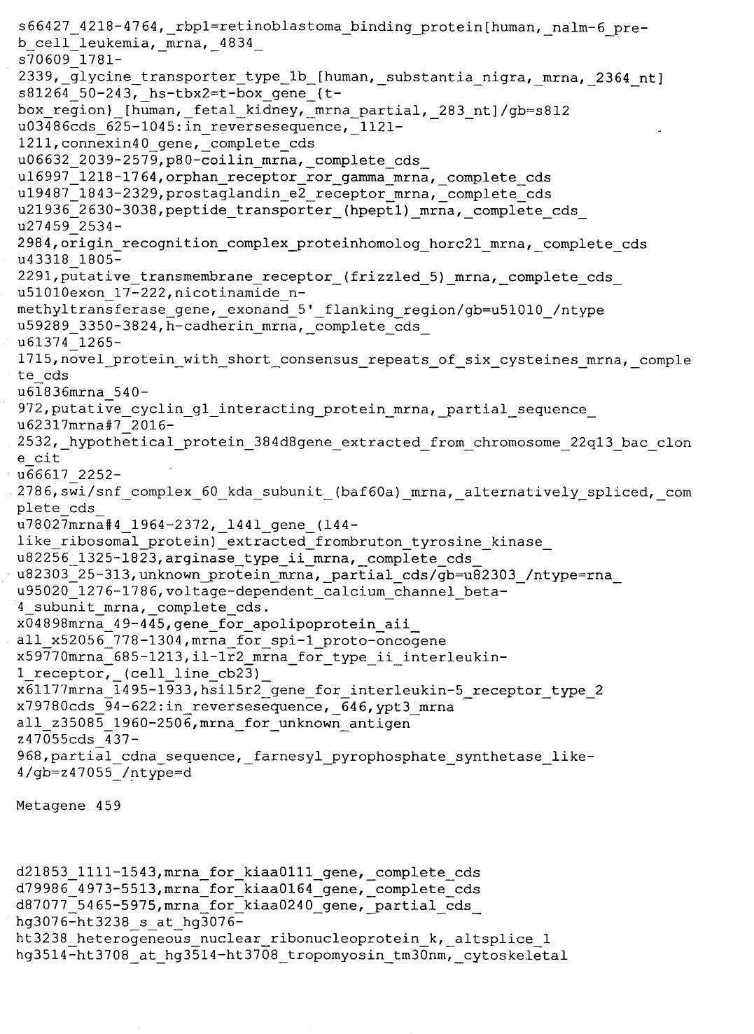

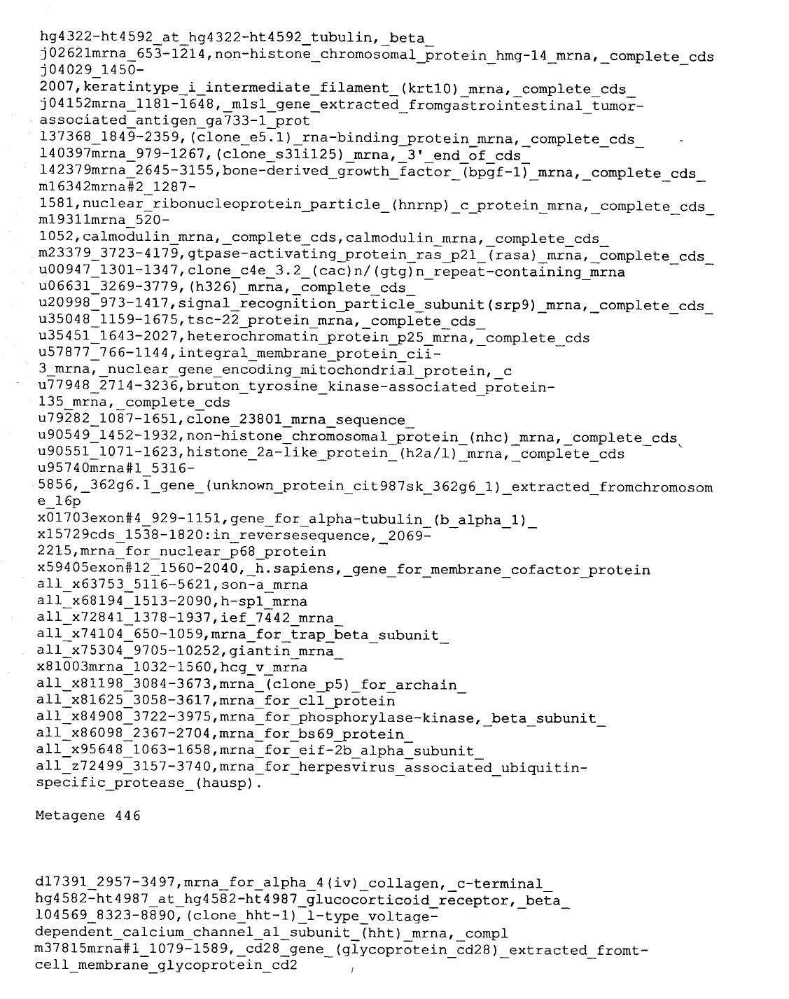

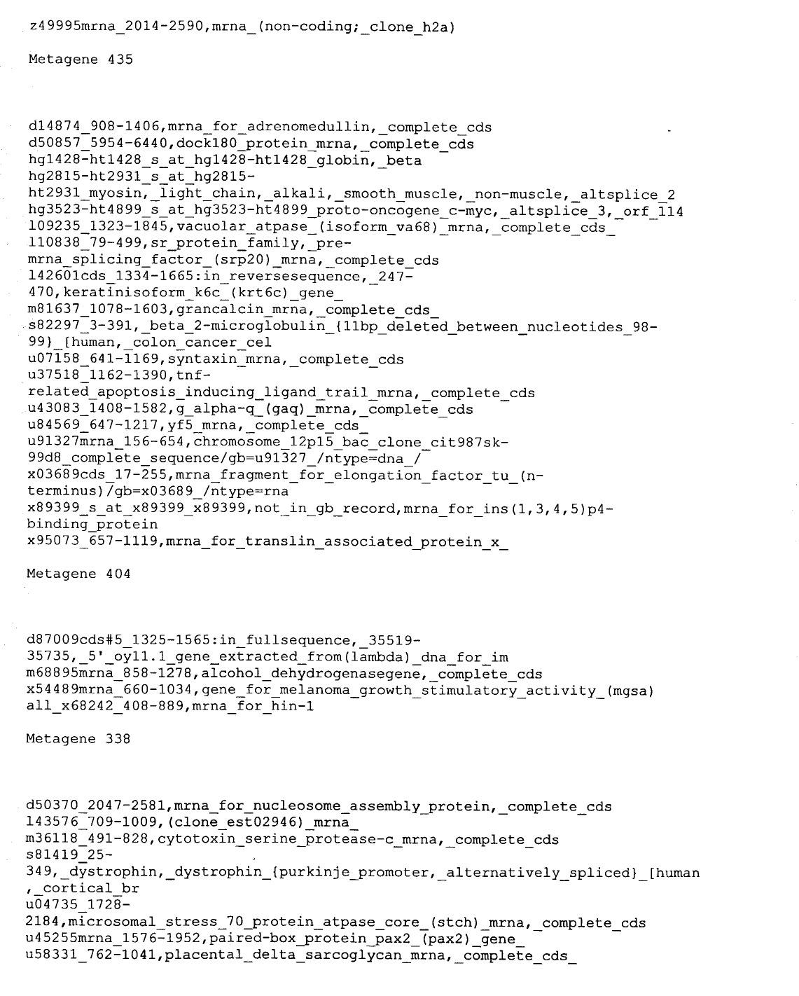

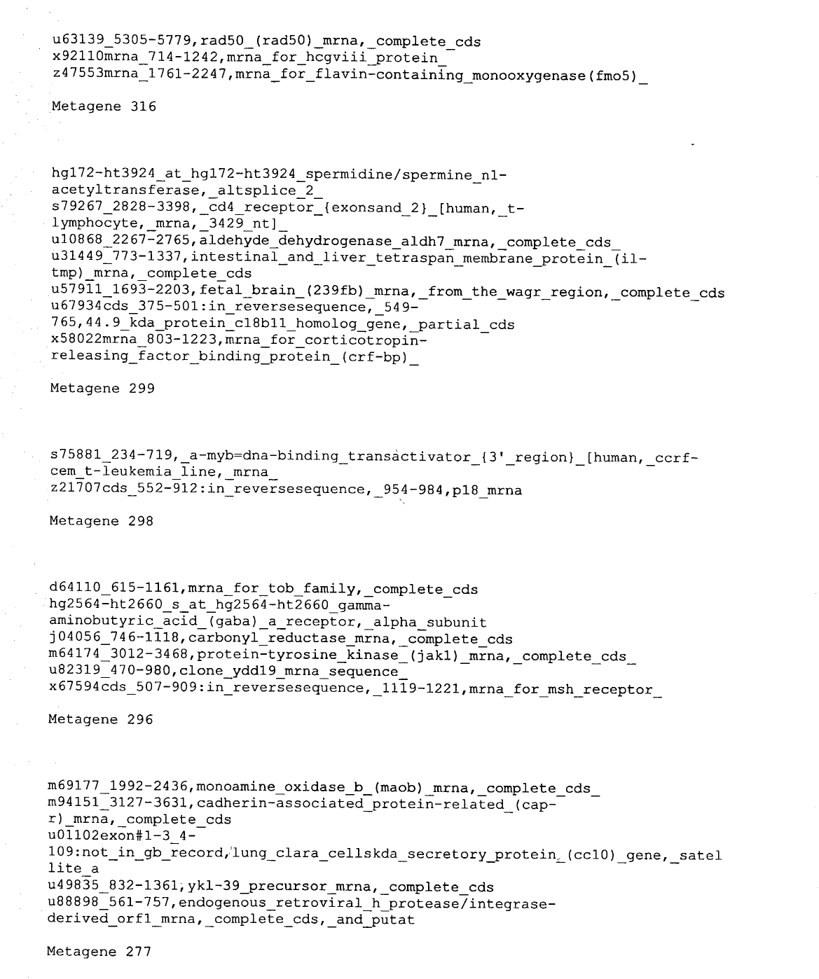

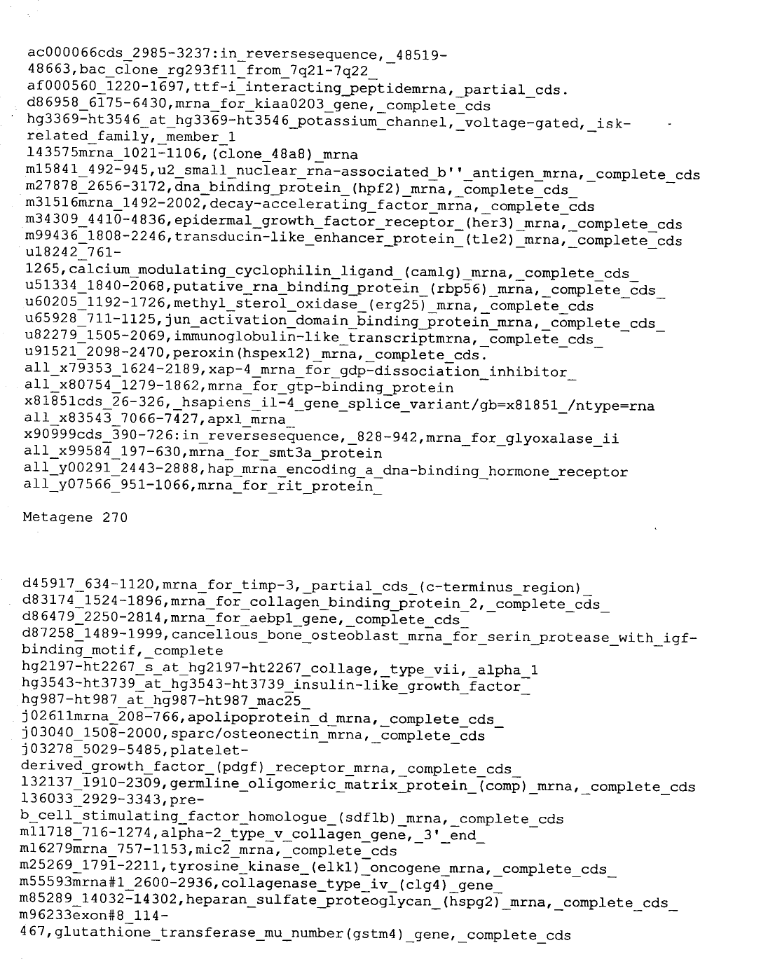

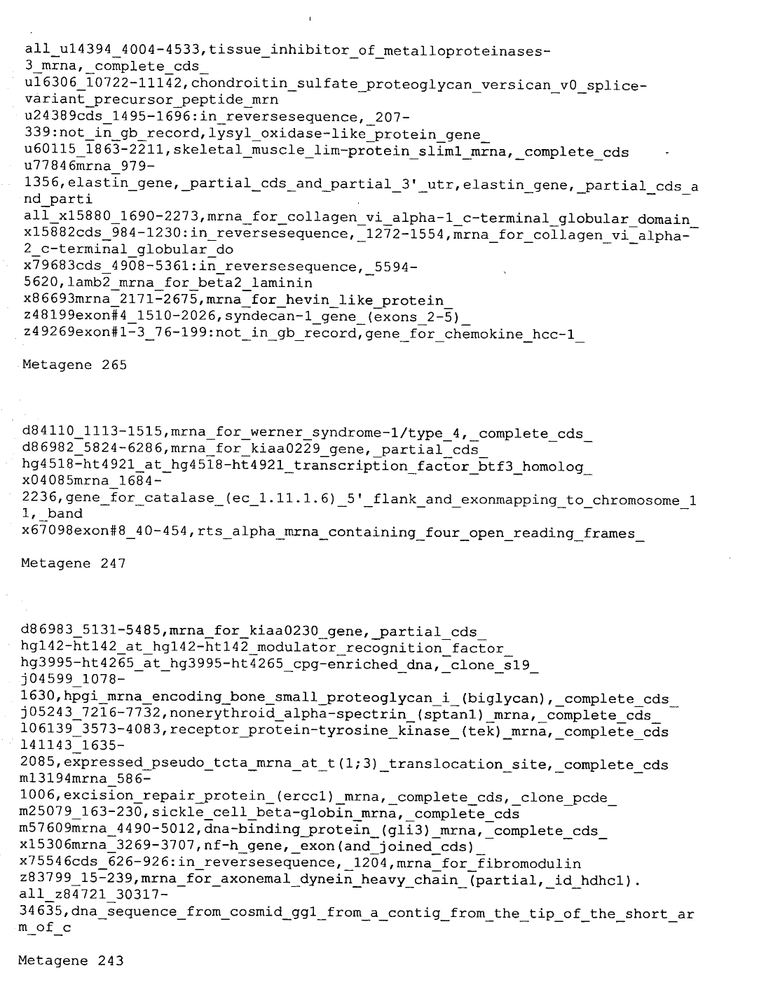

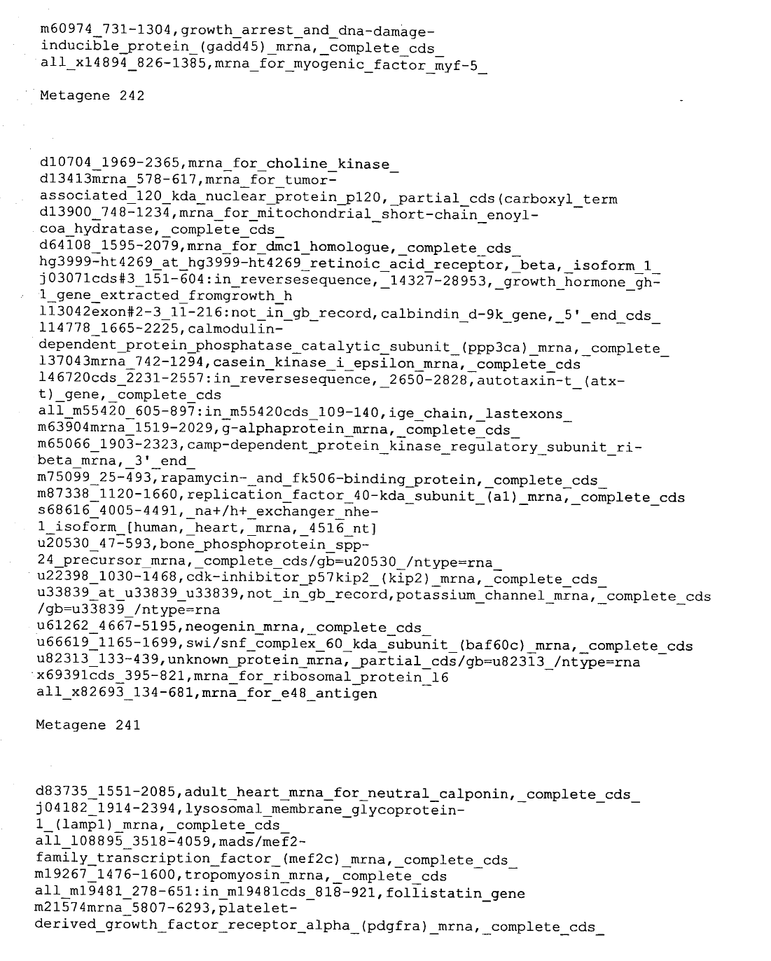

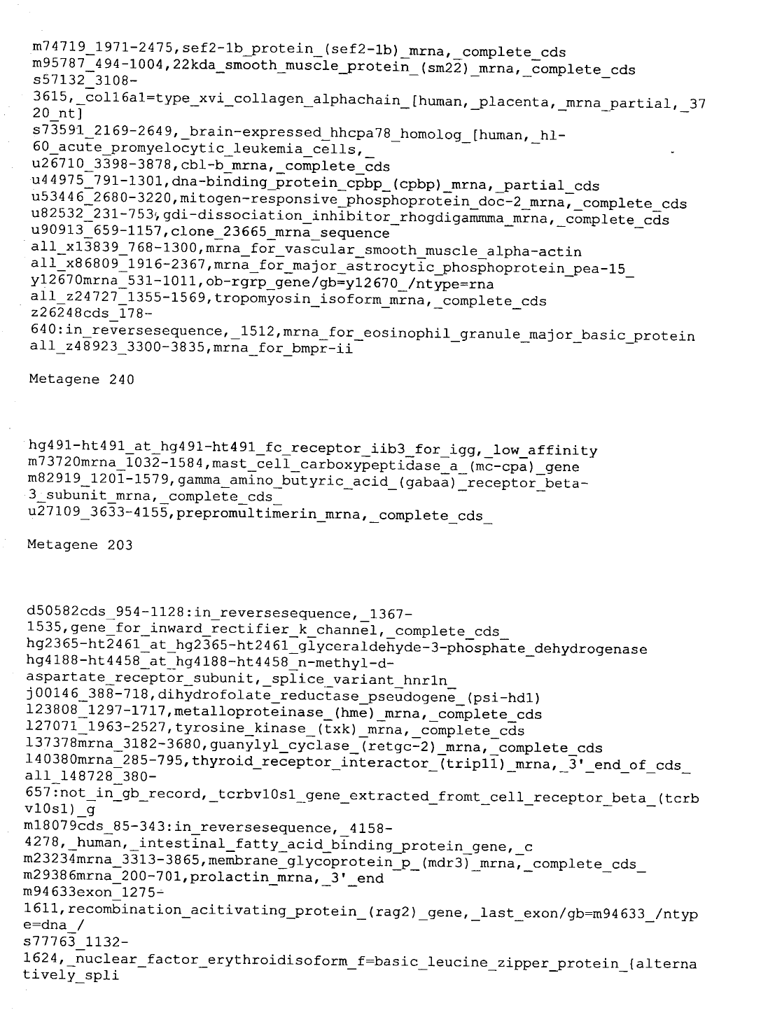

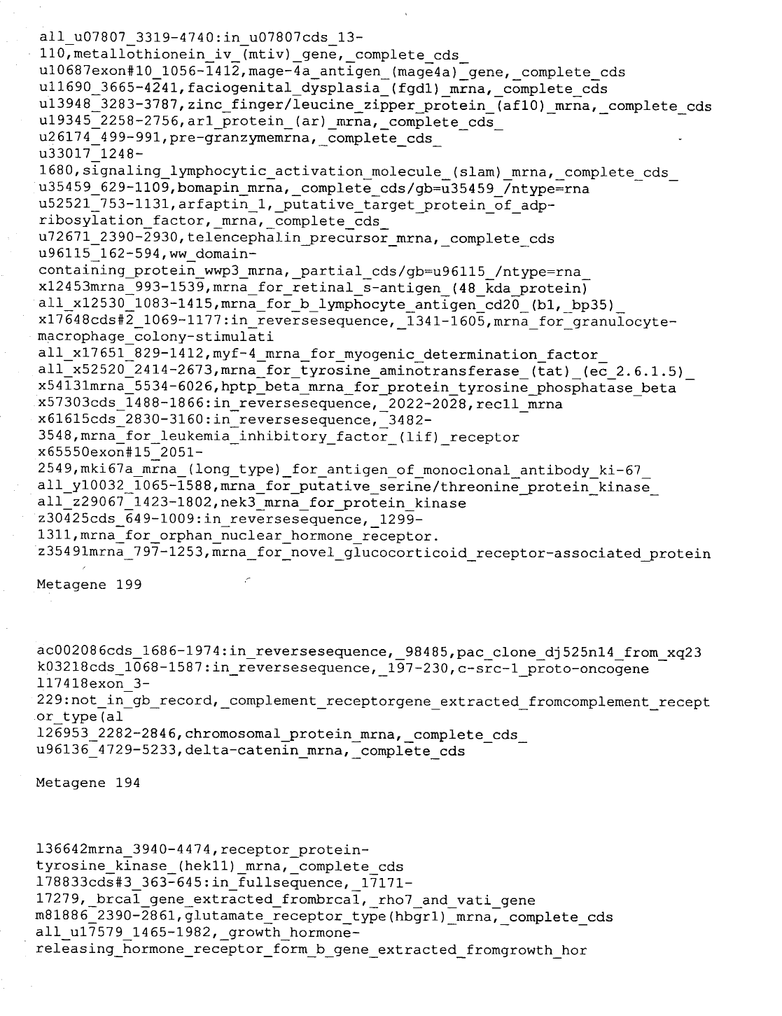

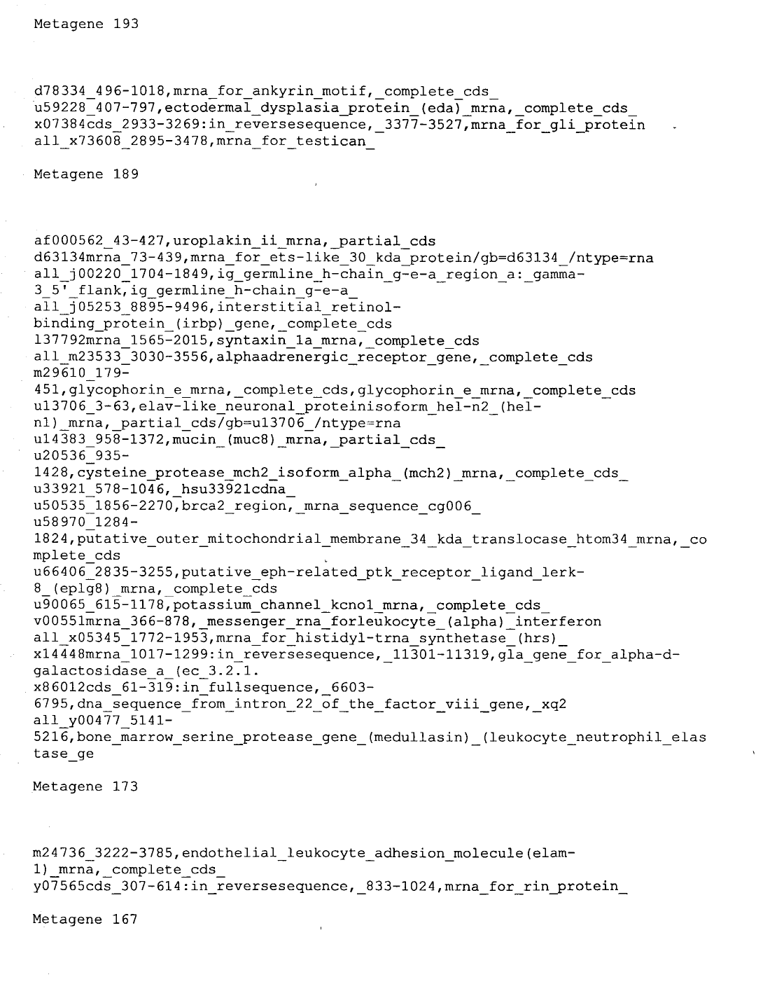

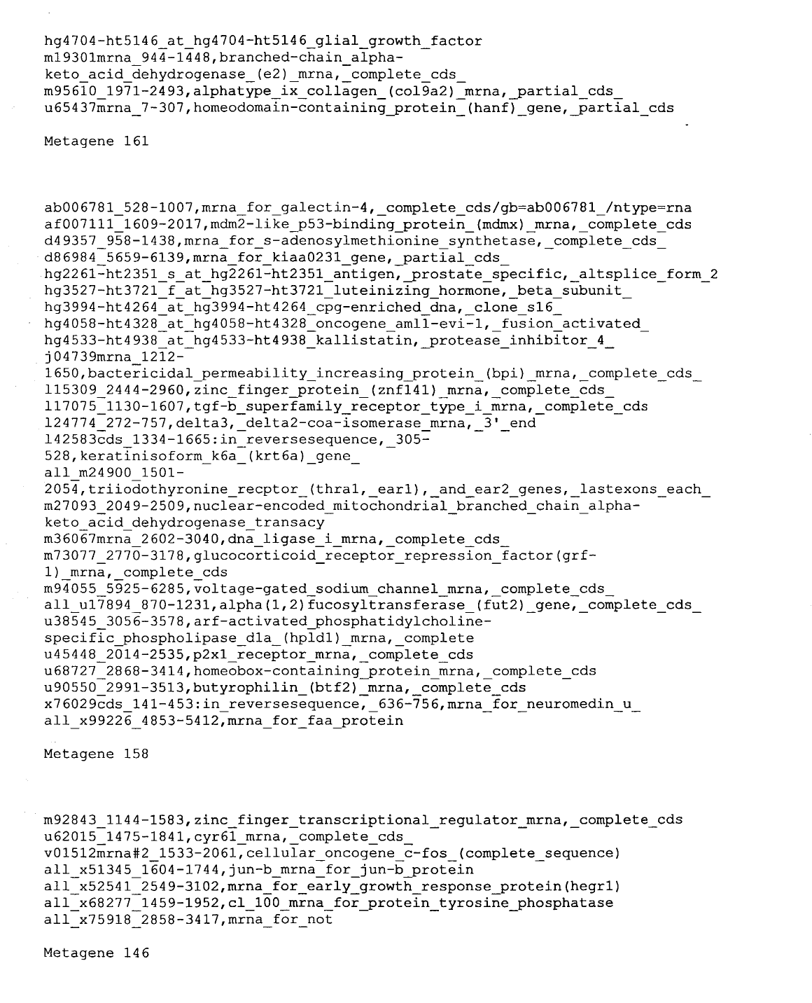

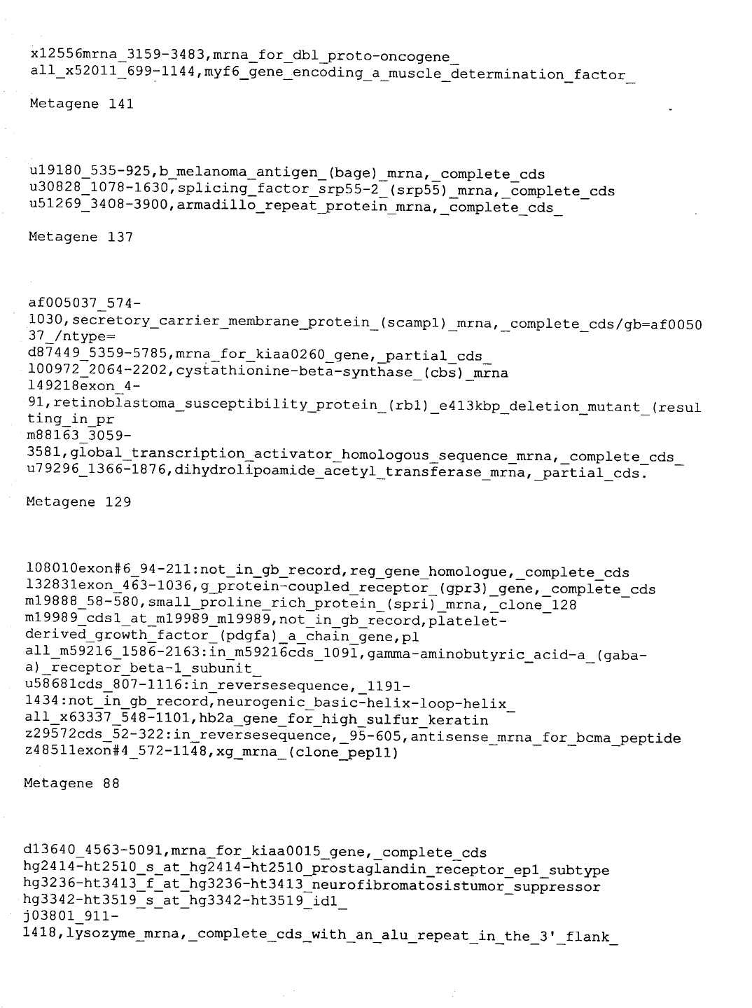

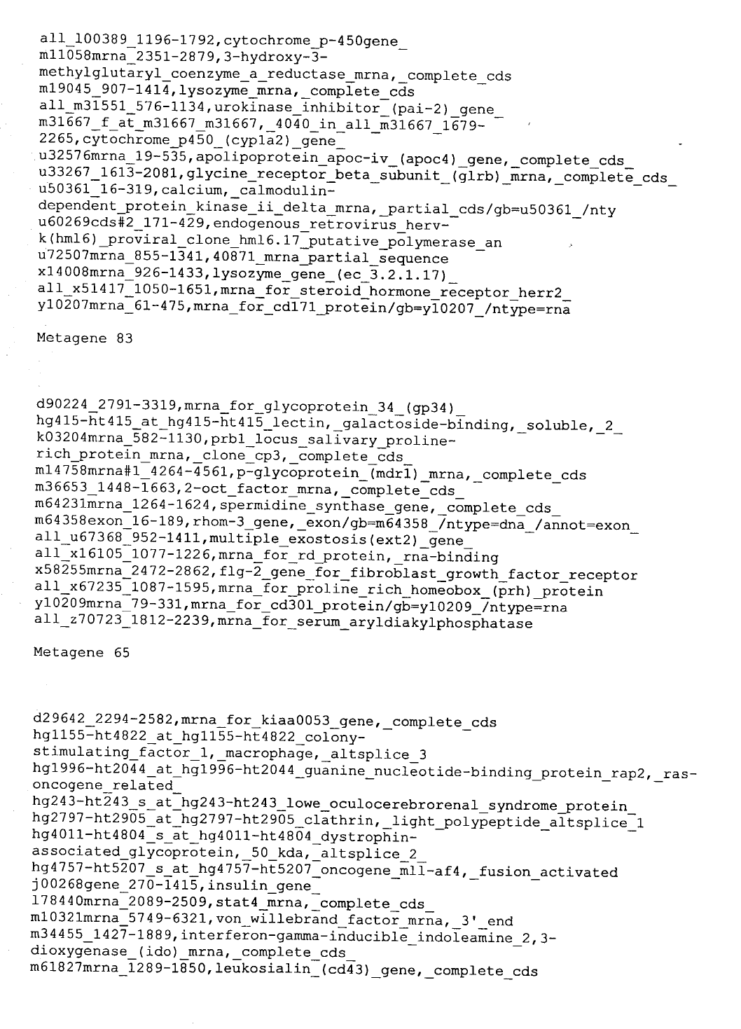

The metagene predictor has dimension p=491: the analysis generated trees based on a Bayes' factor threshold of 3 on the log scale, allowing up to 10 splits of the root node and then up to 4 at each of nodes 1 and 2. Some pertinent summaries appear in the following figures. FIGS. 1 and 2 display 3-D and pairwise 2-D scatterplots of three of the key metagenes, all clearly strongly related to the ER status and also correlated. However, there are in fact five or six metagenes that quite strongly associate with ER status and it is evident that they reflect multiple aspects of this major biological pathway in breast tumors. In the study reported in West et al (2001), Bayesian probit regression models were utilized with singular factor predictors which identified a single major factor predictive of ER. That analysis identified ER negative tumors 16, 40 and 43 as difficult to predict based on the gene expression factor model; the predictive probabilities of ER positive versus negative for these cases were near or above 0.5, with very high uncertainties reflecting real ambiguity.

In contrast to the more more traditional regression models, the current tree model identifies several metagene patterns that together combine to define an ER profile of tumors, and that when displayed as in FIGS. 1 and 2 isolate these three cases as quite clearly consistent with their designated ER negative status in some aspects, yet conflicting and much more in agreement with the ER positive patterns on others. Metagene 347 is the dominant ER signature; the genes involved in defining this metagene include two representations of the ER gene, and several other genes that are coregulated with, or regulated by, the ER gene. Many of these genes appeared in the dominant factor in the regression prediction. This metagene strongly discriminates the ER 11 negatives from positives, with several samples in the mid-range. Thus, it is no surprise that this metagene shows up as defining root node splits in many high-likelihood trees. This metagene also clearly defines these three cases—16, 40 and 43—as appropriately ER negative. However, a second ER associated metagene, number 352, also defines a significant discrimination. In this dimension, however, it is clear that the three cases in question are very evidently much more consistent with ER positives; a number of genes, including the ER regulated PS2 protein and androgen receptors, play roles in this metagene, as they did in the factor regression; it is this second genomic pattern that, when combined together with the first as is implicit in the factor regression model, breeds the conflicting information that fed through to ambivalent predictions with high uncertainty.

The tree model analysis here identifies multiple interacting patterns and allows easy access to displays such as those shown in FIGS. 1 to 3 that provide insights into the interactions, and hence to interpretation of individual cases. In the full tree analysis, predictions based on averaging multiple trees are in fact dominated by the root level splits on metagene 347, with all trees generated extending to two levels where additional metagenes define subsidiary branches. Due to the dominance of metagene 347, the three interesting cases noted above are perfectly in accord with ER negative status, and so are well predicted, even though they exhibit additional, subsidiary patterns of ER associated behaviour identified in the figures. FIG. 6 displays summary predictions. The 9 validation cases are predicted based on the analysis of the full set of 40 training cases. Predictions are represented in terms of point predictions of ER positive status with accompanying, approximate 90% intervals from the average of multiple tree models. The training cases are each predicted in an honest, cross-validation sense: each tumor is removed from the data set, the tree model is then refitted completely to the remaining 39 training cases only, and the hold-out case is predicted, i.e., treated as a validation sample. Excellent predictive performance is observed for both these one-at-a-time honest predictions of training samples and for the out of sample predictions of the 9 validation cases. One ER negative, sample 31, is firmly predicted as having metagene expression patterns completely consistent with ER positive status. This is in fact one of the three cases for which the two laboratory tests conflicted. The other two such cases, however agree with the initial ER negative test result—number 33, for which the predictions firmly agree with the initial ER negative test result, and number 14, for which the predictions agree with the initial ER positive result though not quite so forcefully. The lack of conformity of expression patterns in some cases (Case 8, 11 and 7) are due to major distortions in the data on the DNA microarray due to hybridization problems.

What is claimed is:

1. A classification tree model incorporating Bayesian analysis for the statistical prediction of binary outcomes.

2. The tree model of claim 1, wherein the prediction of a binary outcome is dependent on the interaction of data comprising at least two predictor variables.

3. The tree model of claim 2, wherein the data arises by case control design such that the number of 0/1 values in the response data is fixed by design.

4. The tree model of claim 3, such that the case control design assesses association between predictors and binary outcome with nodes of a tree.

5. The tree model of claim 4, such that the Bayesian analysis comprises using sequences of Bayes factor based tests of association to rank and select predictors that define a node split.

6. The tree model of claim 5, further comprising the forward generation of at least one class of trees with high marginal likelihood, wherein the prediction of said class of trees is conducted using principles of model averaging.

7. The tree model of claim 6, wherein the principle of model averaging comprises the steps of:
weighted prediction of a tree by determining its implied posterior probability by a score;
evaluation of the score to exclude unlikely trees;
evaluation of the posterior and predictive distribution at each node and leaf of a tree; and
application of said posterior and predictive distribution to the evaluation o of each tree and the averaging of predictions across trees for future predictive cases.

8. The tree model of claim 1 or 2, wherein the binary outcome is a clinical state.

9. The tree model of claim 1 or 2, wherein the binary outcome is a physiological state.

10. The tree model of claim 1 or 2, wherein the binary outcome is a physical state.

11. The tree model of claim 1 or 2, wherein the binary outcome is a disease state.

12. The tree model of claim 1 or 2, wherein the binary outcome is a risk group.

13. The tree model of claim 1 or 2, wherein the data is biological data.

14. The tree model of claim 1 or 2, wherein the data is statistical data.

15. The tree model of claim 1 or 2, wherein the binary outcome is estrogen receptor status.

16. Metagenes obtained using the tree model of claim 1 or 2, such that the metagenes characterize multiple patterns of genes predictive of estrogen receptor status.

17. Genes predictive of estrogen receptor status obtained using the tree model of claims 1 or 2.
